SEQUENCE LISTING (734 sequences)

SEQ ID NO: 1 - RNA, 132 nt, synthetic construct
  source 1..132: mol_type = other RNA
gtttgtttacttggcgagacttggagctgaggtcatttggagctgtttaatactgaagagctgttgagcactggaaagtgctgtgtaaccctggaaaagaaccgtgtaacgctgcagaagtgtgtggtagct

SEQ ID NO: 2 - RNA, 629 nt, synthetic construct
  source 1..629: mol_type = other RNA
agagccacgcggcacgcccgggaggctttctctggctggtaaccgctactcccggacaccagaccaccgccttccgtacacaggggcccgcatcccaccctcccggacctaagagcctgggtcccctgtttccggaggtccgcttcccggcccccagattctggcatcccagccctcagtgtccaagacccaggcagcccgggtccccgcctcccggatccaggcgtccgggatctgcgccaccagaacctagcctcctgcagacctccgccatctgggggcactcaacctcctggagccaagggccccacgtcccacccagagaaactctcgtattcccagctcctagggccaaggaacccgggcgctccgaactcccagctttcggacatctggcacacggggcagagcagagaagcctcagcgcccagcctggggaatttaaacactccagcttccaagagccaaggaacttcagtgctgtgaactcacaactctaaggagccctccaaagttccagtctccaggtgctgttactcaactcagtcctaggaacgtcgggtcctgggaaggagcccaagcgctcccagccagcttccaggcgctaagaaaccccggtgcttcccatc

SEQ ID NO: 3 - RNA, 169 nt, synthetic construct
  source 1..169: mol_type = other RNA
agtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcg

SEQ ID NO: 4 - RNA, 311 nt, synthetic construct
  source 1..311: mol_type = other RNA
ggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaag

SEQ ID NO: 5 - RNA, 336 nt, synthetic construct
  source 1..336: mol_type = other RNA
ggacaagcacttgccaccaccgtcactcagccctgggcgtagccggacaggaggagagcagtgatgcggatgggtacccgggcacaccagccctcagagacctgagctcttctggccacgtggaacctcgaacccgagctcctgcagaagtggccctggagattgagggtccctggacactccctatggagatccggggagctaggatggggaacctgccacagccagaactgaggggctggccccaggcagctcccagggggtagaacggccctgtgcttaagacactcctgctgccccgtctgagggtggcaattaaagttgcttcacatcctc

SEQ ID NO: 6 - RNA, 500 nt, synthetic construct
  source 1..500: mol_type = other RNA
atggctatcctttaatgatgcgtgtggaatgtgtgtgtgtgctcaggcaattattttgctaagaatgtgaattcaagtgcagctcaatactagcttcagtataaaaactgtacagatttttgtatagctgataagattctctgtagagaaaatacttttaaaaaatgcaggttgtagctttttgatgggctactcatacagttagattttacagcttctgatgttgaatgttcctaaatatttaatggtttttttaatttcttgtgtatggtagcacagcaaacttgtaggaattagtatcaatagtaaattttgggttttttaggatgttgcatttcgtttttttaaaaaaaattttgtaataaaattatgtatattatttctattgtctttgtcttaatatgctaagttaattttcactttaaaaaagccatttgaagaccagagctatgttgatttttttcggtatttctgcctagtagttcttagacacagttgac

SEQ ID NO: 7 - RNA, 146 nt, synthetic construct
  source 1..146: mol_type = other RNA
atggctatcctttaatgatgcgtgtggaatgtgtgtgtgtgctcaggcaattattttgctaagaatgtgaattcaagtgcagctcaatactagcttcagtataaaaactgtacagatttttgtatagctgataagattctctgtag

SEQ ID NO: 8 - RNA, 238 nt, synthetic construct
  source 1..238: mol_type = other RNA
aagccgggtgggggtgagggtagcccttgagccctgtccctgcggctgtgagagcagcaggaccctgggccagttccagagacctgggggtgtgtctgggggtggggtgtgagtgcgtatgaaagtgtgtgtctgctggggcagctgtgcccctgaatcatgggcacggagggccgcccgccacgccccgcgctcaactgctcccgtggaagattaaagggctgaatcatggtgctga

SEQ ID NO: 9 - RNA, 100 nt, synthetic construct
  source 1..100: mol_type = other RNA
ggacacatggttaccacctggccctgagtgcagtcagactgggacaaaggagccgtgaaacacgcaaggagcttctggcttctcagctttgagacctggg

SEQ ID NO: 10 - RNA, 212 nt, synthetic construct
  source 1..212: mol_type = other RNA
gctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaag

SEQ ID NO: 11 - RNA, 152 nt, synthetic construct
  source 1..152: mol_type = other RNA
ggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaag

SEQ ID NO: 12 - RNA, 61 nt, synthetic construct
  source 1..61: mol_type = other RNA
gctctttggagcacagagaactttgatttttaagcttctacagatatggtcctagagactg

SEQ ID NO: 13 - RNA, 111 nt, synthetic construct
  source 1..111: mol_type = other RNA
gctctttggagcacagagaactttgatttttaagcttctacagatatggtcctagagactggggtcctcagacactcccgctgtatgtggtgagcttggctggaaacttgc

SEQ ID NO: 14 - RNA, 181 nt, synthetic construct
  source 1..181: mol_type = other RNA
gctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcc

SEQ ID NO: 15 - RNA, 201 nt, synthetic construct
  source 1..201: mol_type = other RNA
gctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggt

SEQ ID NO: 16 - RNA, 141 nt, synthetic construct
  source 1..141: mol_type = other RNA
ctttgatttttaagcttctacagatatggtcctagagactggggtcctcagacactcccgctgtatgtggtgagcttggctggaaacttgccatagccaggactgaggggctggccccaggacgctctcaagaaatggggc

SEQ ID NO: 17 - RNA, 151 nt, synthetic construct
  source 1..151: mol_type = other RNA
ctttgatttttaagcttctacagatatggtcctagagactggggtcctcagacactcccgctgtatgtggtgagcttggctggaaacttgccatagccaggactgaggggctggccccaggacgctctcaagaaatggggctactgtggtt

SEQ ID NO: 18 - RNA, 171 nt, synthetic construct
  source 1..171: mol_type = other RNA
ctttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcac

SEQ ID NO: 19 - RNA, 125 nt, synthetic construct
  source 1..125: mol_type = other RNA
tctacagatatggtcctagagactggggtcctcagacactcccgctgtatgtggtgagcttggctggaaacttgccatagccaggactgaggggctggccccaggacgctctcaagaaatggggc

SEQ ID NO: 20 - RNA, 135 nt, synthetic construct
  source 1..135: mol_type = other RNA
tctacagatatggtcctagagactggggtcctcagacactcccgctgtatgtggtgagcttggctggaaacttgccatagccaggactgaggggctggccccaggacgctctcaagaaatggggctactgtggtt

SEQ ID NO: 21 - RNA, 115 nt, synthetic construct
  source 1..115: mol_type = other RNA
tggtcctagagactggggtcctcagacactcccgctgtatgtggtgagcttggctggaaacttgccatagccaggactgaggggctggccccaggacgctctcaagaaatggggc

SEQ ID NO: 22 - RNA, 125 nt, synthetic construct
  source 1..125: mol_type = other RNA
tggtcctagagactggggtcctcagacactcccgctgtatgtggtgagcttggctggaaacttgccatagccaggactgaggggctggccccaggacgctctcaagaaatggggctactgtggtt

SEQ ID NO: 23 - RNA, 101 nt, synthetic construct
  source 1..101: mol_type = other RNA
ggggtcctcagacactcccgctgtatgtggtgagcttggctggaaacttgccatagccaggactgaggggctggccccaggacgctctcaagaaatggggc

SEQ ID NO: 24 - RNA, 111 nt, synthetic construct
  source 1..111: mol_type = other RNA
ggggtcctcagacactcccgctgtatgtggtgagcttggctggaaacttgccatagccaggactgaggggctggccccaggacgctctcaagaaatggggctactgtggtt

SEQ ID NO: 25 - RNA, 121 nt, synthetic construct
  source 1..121: mol_type = other RNA
acaagtaagggcttgaaaatgatactggcaagatacgattggctctagatctacattcttcaaaaaaaaaaattggcttaactgtttcatctttaagtagcattttgctgccatttgtatt

SEQ ID NO: 26 - RNA, 393 nt, synthetic construct
  source 1..393: mol_type = other RNA
gggacagggtcagggcagcccagggctcctggcttcagcaggaagtgaacaggctcagggaactggaggaagcgaagcatcaaggccagaggaggccacatgctgaccagcctgatgaggcaagagcctgcccctgccaccgccccgacccctctcctctctgcaagagcctgcctctgccaccgccccgaccccctctcctctcagcaagggatgggcctctctgcctcgcccacccctcagccctcctcccagccatctcctcttccctaaggcctctgtctccatagctctggtttccctgggcctcagtcctccccaccctccttcctctgtctccctgtcactaatgtgaggtttctttgtgcacattaaagtcttctttcagcatca

SEQ ID NO: 27 - RNA, 55 nt, synthetic construct
  source 1..55: mol_type = other RNA
agggtttcgtctttagatcttctggtccccacagactcagagagaacccgccacc

SEQ ID NO: 28 - RNA, 500 nt, synthetic construct
  source 1..500: mol_type = other RNA
gcgcaaaggggctgaaagaacatggacttgtatatttgtacaaaaaaaaagttttatttttctaaaaaaagaaaaaagaagaaaaaatttaaagggtgtacttatatccacactgcacactgcctggcccaaaacgtcttattgtggtaggatcagccctcattttgttgcttttgtgaactttttgtaggggacgagaaagatcattgaaattctgagaaaacttcttttaaacctcacctttgtggggtttttggagaaggttatcaaaaatttcatggaaggaccacattttatatttattgtgcttcgagtgactgaccccagtggtatcctgtgacatgtaacagccaggagtgttaagcgttcagtgatgtggggtgaaaagttactacctgtcaaggtttgtgttaccctcctgtaaatggtgtacataatgtattgttggtaattattttggtacttttatgatgtatatttattaaacagatttttacaaa

SEQ ID NO: 29 - RNA, 445 nt, synthetic construct
  source 1..445: mol_type = other RNA
aattgatcagggaccatgaaaagaaacttgtgcttcaccgaagaaaaatatctaaacatcgaaaaacttaaatattatggaaaaaaaacattgcaaaatataaaataaataaaaaaaggaaaggaaactttgaaccttatgtaccgagcaaatgccaggtctagcaaacataatgctagtcctagattacttattgatttaaaaacaaaaaaacacaaaaaaatagtaaaatataaaaacaaattaatgttttatagaccctgggaaaaagaattttcagcaaagtacaaaaatttaaagcattcctttctttaattttgtaattctttactgtggaatagctcagaatgtcagttctgttttaagtaacagaattgataactgagcaaggaaacgtaatttggattataaaattcttgctttaataaaaattccttaaacagtg

SEQ ID NO: 30 - RNA, 500 nt, synthetic construct
  source 1..500: mol_type = other RNA
tccccagacctctgtccctgttcccctccactcctcccctcactcccctgctcccccgaccacctcctcctctgcctcaaagactcttgtcctcttgtccctcctgagaaaaaagaaaacgaaaagtggggtttttttctgttttctttttttcccctttccccctgcccccacccacggggcctttttttggaggtgggggctggggaatgaggggctgaggtcccggaagggattttatttttttgaattttaattgtaacatttttagaaaaagaacaaaaaaagaaaaaaaaaagaaagaaacacagcaactgtagatgctcctgttcctggttcccgctttccacttccaaatccctcccctcaccttcccccactgccccccaagttccaggctcagtcttccagccgcctggggagtctctacctgggcccaagcaggtgtggggcctccttctgggcttttcttctgaatttagaggatttctagaacgtgg

SEQ ID NO: 31 - RNA, 351 nt, synthetic construct
  source 1..351: mol_type = other RNA
gcgcaaaggggctgaaagaacatggacttgtatatttgtacaaaaaaaaagttttatttttctaaaaaaagaaaaaagaagaaaaaatttaaagggtgtacttatatccacactgcacactgcctggcccaaaacgtcttattgtggtaggatcagccctcattttgttgcttttgtgaactttttgtaggggacgagaaatgaggggctgaggtcccggaagggattttatttttttgaattttaattgtaacatttttagaaaaagaacaaaaaaagaaaaaaaaaagaaagaaacacagcaactgtagatgctcctgttcctggttcccgctttccacttccaaatcc

SEQ ID NO: 32 - RNA, 200 nt, synthetic construct
  source 1..200: mol_type = other RNA
tggatgtacatcattcccgtcgtcctgttcctcatgatgtcaggagcgccagacaccgggggccagggtgggggtgggggtgggggtggtggtgggggtagtggccggtgagggcccaggctggtcagcgtcccgtcttgcacacccaggggcctccctttctgctggagtcccctgtgtcctcagccatcccaagaagg

SEQ ID NO: 33 - RNA, 250 nt, synthetic construct
  source 1..250: mol_type = other RNA
gcaaaactgccgcaagtctgcagcccggcgccaccatcctgcagcctcctcctgaccacggacgtttccatcaggttccatcccgaaaatctctcggttccacgtccccctggggcttctcctgacccagtccccgtgccccgcctccccgaaacaggctactctcctcggccccctccatcgggctgaggaagcacagcagcatcttcaaacatgtacaaaatcgattggctttaaacacccttcacat

SEQ ID NO: 34 - RNA, 250 nt, synthetic construct
  source 1..250: mol_type = other RNA
tgaaaaccaaaatccatgcaaatgaaatgtaattggcacgaccctcacccccaaatcttacatctcaattcccatcctaaaaagcactcatactttatgcatccccgcagctacacacacacaacacacagcacacgcatgaacacagcacacacacgagcacagcacacacacaaacgcacagcacacacagcacacagatgagcacacagcacacacacaaacgcacagcacacacacgcacacacat

SEQ ID NO: 35 - RNA, 1000 nt, synthetic construct
  source 1..1000: mol_type = other RNA
cacacacacgcacacacatgcacacacagcacacaaacgcacggcacacacacgcacacacatgcacacacagcacacacacaaacgcacagcacacacaaacgcacagcacacacgcacacacagcacacacacgagcacacagcacacaaacgcacagcacacgcacacacatgcacacacagcacacacactagcacacagcacacacacaaagacacagcacacacatgcacacacagcacacacacgcgaacacagcacacacgaacacagcacacacagcacacacacaaacacagcacacacatgcacacagcacacgcacacacagcacacacatgaacacagcacacagcacacacatgcacacacagcacacacgcatgcacagcacacatgaacacagcacacacacaaacacacagcacacacatgcacacacagcacacacactcatgcgcagcacatacatgaacacagctcacagcacacaaacacgcagcacacacgttgcacacgcaagcacccacctgcacacacacatgcgcacacacacgcacacccccacaaaattggatgaaaacaataagcatatctaagcaactacgatatctgtatggatcaggccaaagtcccgctaagattctccaatgttttcatggtctgagccccgctcctgttcccatctccactgcccctcggccctgtctgtgccctgcctctcagaggagggggctcagatggtgcggcctgagtgtgcggccggcggcatttgggatacacccgtagggtgggcggggtgtgtcccaggcctaattccatctttccaccatgacagagatgcccttgtgaggctggcctccttggcgcctgtccccacggcccccgcagcgtgagccacgatgctccccataccccacccattcccgatacaccttacttactgtgtgttggcccagccagagtgaggaaggagtttggccacattggagatggc

SEQ ID NO: 36 - RNA, 641 nt, synthetic construct
  source 1..641: mol_type = other RNA
gtcgcatgaatgccatctctggctggcaggccttctttccagtccatttccaggagttcaatcctgccctgtcaccacagagatcacccccagggcccccgggggctggccctgaccccccctcccctcctggtgctgacccctcccggggggctcctataggggggagatttgaccggcaggcttctgcggagggctgcttctacaacgctgactacctggcggcccgagcccggctggcaggtgaactggcaggccaggaagaggaggaagccctggaggggctggaggtgatggatgttttcctccggttctcagggctccacctctttcgggccgtagagccagggctggtgcagaagttctccctgcgagactgcagcccacggctcagtgaagaactctaccaccgctgccgcctcagcaacctggaggggctagggggccgtgcccagctggctatggctctctttgagcaggagcaggccaatagcacttagcccgcctgggggccctaacctcattacctttcctttgtctgcctcagccccaggaagggcaaggcaagatggtggacagatagagaattgttgctgtattttttaaatatgaaaatgttattaaacatgtcttctgccaaa

SEQ ID NO: 37 - RNA, 100 nt, synthetic construct
  source 1..100: mol_type = other RNA
ttctggaatctgtgctctgggggctgtgccgggtagagagggcagtgggaggtaagagctcttcacccttcaccaccttctccacccagcatggccggca

SEQ ID NO: 38 - RNA, 656 nt, synthetic construct
  source 1..656: mol_type = other RNA
gtttagctttgtgttagcttatacatactaaaacctttaaaaagcttttcttctcaattgatttttttcttttagaagccatggtgtctcaaccttttggggacctaacttctaaacattctaatagtttgccttaatttttcttctgctttcttactaaaaatgaagacattcaatactaatcttgctggaagaagccttaaccaagcaaacttctcatttctctggtgaaaactgctgccaaaaccacttgttaaaaattgtacagagcctgtagaaaatatagaagattcattggatgttggcctagttctgtgtggaagactagtgattttgttgtttttagataactaaatcgacaacaaatcacagtctgccatatggcacaggccatgcctctacaggacaaatgattggtgctgtaaaatgcagcatttcacaccttactagcattctttgtcttttctaccaaatattaacaactttcaattccgttttcttaattctgttctactaatgtccgatttactactcatcatttttcttgacacttaacattgctttaatttgtaattgctaatggtttttgaactcttctaattgtaatggacgtgtttatcattttaatttagcattgaaattgtcttgatgttgat

SEQ ID NO: 39 - RNA, 1076 nt, synthetic construct
  source 1..1076: mol_type = other RNA
cctgaggctgggttggagcagccctcctgtgcctgaggccagctcccaggcccttggatcaccgcgggaggaaccctcaggatgggtggagcctccaggctatgggcattgcctgcctgatgccagcaccacctgggctgggccctgggcttggctcgagttctcctgctggtgaggctccggatctcaggagcagccctgagtctgcttcccaggctgcccctgccaggcctgcagcctccccagccagggctgctctctgctgtccccattcagtgccctggcccctgcattcatgccccccacaccccctcaggccctgtgcctggactttggggctggcagctgaagccttgagatcctgggccagctgccggcacacagctaggcagactctcccaccaggtgcccctgcccaggcctcctaatcgggggcagacaggcagggagggtgtggctgggctgggctgggcggggcggcctggggcaggggtgtggcccctaaatgtccccaacctcagagggacctagagtcctgagcctccagtagcttctctgggcctggcagaggtaagggggaggcaaccctggagtgtctggaggcccatggctggctgaaccctggatgccttttcttccgcgtccccatgaatgaaagctgtctgggccttcattctgcagacagggacaaacagctccatgctgtttgtcctcccagtgcagccgtgctgggagggtctgggggagcttcctacaaggagagactcctgctgctttggaaaactgagaaaaaataggggtctaaccctctcctcccattttacaagtggggaaatgaggcgtgaaaggagaggcgtctgggttactccgtgggtctggggtccagggaagggcctgtatgggggagggagctgggaggggacggtgtctggctctacccctgtggggtggggaggtggggctcccctgtatcacaggacatcccccctgagaggtccctcatatgtctgggtcctgtgggtgggggactaactgcgcaatgtagttaggtgctcaataaacggagttgccgctga

SEQ ID NO: 40 - RNA, 500 nt, synthetic construct
  source 1..500: mol_type = other RNA
tgttcagaggtccctgtcttctgtccccatcttcctgccgatagctatcccctgtatgatgttggatgctcctcacatgctgagtttccagccttttctgaaactcagtagctggggagagggcagggaggcttcctgggccttccagcctccttccccacctccttcccaaaccctcttgggaactcctcagggacaactactgctgagtttgggtgcaccctaagatggaggccaggtagcaatggggccggcctcagagagagcgctgtgtgtgtgtgtgtgtgtgtgtgtgtgtgtgtgtgtcggcctcagagcgcgctgtgtgtgtgtgtgtgtgtgtcagcctcagagcgcgcgctgtgtgtgtgtgtgtgtgtgcgtgcttgtgaccctgtattgtttgataggatccattcagtttccccaagtacctgttttcattcccatttttcccattgtttaaaaccatcacttttttgtctttgggaaaccacagg

SEQ ID NO: 41 - RNA, 1000 nt, synthetic construct
  source 1..1000: mol_type = other RNA
cttttcacaagatggacccttcatttcaagcttaggctggcgttacttttgctgtctagtcaggactaatcacggtgtttcagtgcggagtgccaagagtcctatcctgacgtcaggctctgggtgtcaacctctgacttattctgcagatgctctgtgtgtgtgtgtgtgtgtgtgtgtgtgtgtgtgtgtgtgtgtgtgttcggggagagggtggtagcacagggcttgggatatcggcagtgtgggaaatgcgaagcatttctcatcatcatcatctctgctacagtcatgtttctgcatgtcagcgagcgacactgtccctgcctcaggttggaggttttatcagccaaagtgtttttttcatgtatcgttcgttccattcatccactctgtgccttgtcagcctttgaaaggcttggttgctcccaggctgctgttctcagggaccttaaaagggacctggttagtcttggggcagagagtatctacttgggcaccttttcacaagatggacccttcatttcaagcttaggctggcgttacttttgctgtctagtcaggactaatcacggtgtttcagtgcggagtgccaagagtcctatcctgacgtcaggctctgggtgtcaacctctgacttattctgcagatgctctgtgtgtgtgtgtgtgtgtgtgtgtgtgtgtgtgtgtgtgtgtgtgttcggggagagggtggtagcacagggcttgggatatcggcagtgtgggaaatgcgaagcatttctcatcatcatcatctctgctacagtcatgtttctgcatgtcagcgagcgacactgtccctgcctcaggttggaggttttatcagccaaagtgtttttttcatgtatcgttcgttccattcatccactctgtgccttgtcagcctttgaaaggcttggttgctcccaggctgctgttctcagggaccttaaaagggacctggttagtcttggggcagagagtatctacttgggcac

SEQ ID NO: 42 - RNA, 418 nt, synthetic construct
  source 1..418: mol_type = other RNA
gtgtgtgtacatctgggtgtgtgtgagcaagtgtgcagagggtgcgtgtgcacaggtacgggcaggtgcatgtgtgcgagtggctgtgtgagtgggtgtgtgagaaggtgaatgtgagagctggtgtggctgagagtgggtgtggccatgtttgtgtgtctgtcaacacatgaatgtatttcagtgcgtgtgagcagctgtgcacgtatgatgtcagtgggtgtcagcagtgtgtgggtgtgcctgtgtgagcagggatgtgagcatggtatatgtgagtggctgtaagcaggcatgtgtgagtgatatgtgtgagtgggggtgtgacagcaggtgtgtgagcaagtgtatatgtgtgaatgtgtatgtaagcagggatgtgtgtgatataagtgtgtgtgaatgggcataggtgtgt

SEQ ID NO: 43 - RNA, 500 nt, synthetic construct
  source 1..500: mol_type = other RNA
cttttcacaagatggacccttcatttcaagcttaggctggcgttacttttgctgtctagtcaggactaatcacggtgtttcagtgcggagtgccaagagtcctatcctgacgtcaggctctgggtgtcaacctctgacttattctgcagatgctctgtgtgtgtgtgtgtgtgtgtgtgtgtgtgtgtgtgtgtgtgtgtgttcggggagagggtggtagcacagggcttgggatatcggcagtgtgggaaatgcgaagcatttctcatcatcatcatctctgctacagtcatgtttctgcatgtcagcgagcgacactgtccctgcctcaggttggaggttttatcagccaaagtgtttttttcatgtatcgttcgttccattcatccactctgtgccttgtcagcctttgaaaggcttggttgctcccaggctgctgttctcagggaccttaaaagggacctggttagtcttggggcagagagtatctacttgggcac

SEQ ID NO: 44 - RNA, 500 nt, synthetic construct
  source 1..500: mol_type = other RNA
ctaggtgcagagagcccaggccttatgttaaaatcatgcacttgaaaagcaaaccttaatctgcaaagacagcagcaagcattatacggtcatcttgaatgatccctttgaaattttttttttgtttgtttgtttaaatcaagcctgaggctggtgaacagtagctacacacccatattgtgtgttctgtgaatgctagctctcttgaatttggatattggttattttttatagagtgtaaaccaagttttatattctgcaatgcgaacaggtacctatctgtttctaaataaaactgtttacattcattatggggtatgtatgaccttcattttccaagaaatagaactctagcttagaattatggatgctctaaaatgtcagaatgggaactctcctcgaagttctcccaaactcagagacagcactgccttctcctaaatgattattcttttctccctgttttctggtattttctagcatccttctcaccacagcca

SEQ ID NO: 45 - RNA, 326 nt, synthetic construct
  source 1..326: mol_type = other RNA
agggagccatgagggtctgggcttcagagctaggtctttggggaagtcctggctgactgccttagcagtgggggtgggggtgggggcaggggcaggggctttatgtgtttttgcttggggggcgctgggcctagcccagagtagtgcttgctccccctgccttgtcccaccagggaggcagcagactcaggccctccatggtcctctttgtcattttgttgacatgcattcctccttttgtcatcttgttggggggaggggattaaccaaaggccaccctgactttgtttttgtggacacacaataaaagccccgtttatttgtaa

SEQ ID NO: 46 - RNA, 1000 nt, synthetic construct
  source 1..1000: mol_type = other RNA
acacacacacaacacacagcacacgcatgaacacagcacacacacgagcacagcacacacacaaacgcacagcacacacagcacacagatgagcacacagcacacacacaaacgcacagcacacacacgcacacacatgcacacacagcacacaaacgcacggcacacacacgcacacacatgcacacacagcacacacacaaacgcacagcacacacaaacgcacagcacacacgcacacacagcacacacacgagcacacagcacacaaacgcacagcacacgcacacacatgcacacacagcacacacactagcacacagcacacacacaaagacacagcacacacatgcacacacagcacacacacgcgaacacagcacacacgaacacagcacacacagcacacacacaaacacagcacacacatgcacacagcacacgcacacacagcacacacatgaacacagcacacagcacacacatgcacacacagcacacacgcatgcacagcacacatgaacacagcacacacacaaacacacagcacacacatgcacacacagcacacacactcatgcgcagcacatacatgaacacagctcacagcacacaaacacgcagcacacacgttgcacacgcaagcacccacctgcacacacacatgcgcacacacacgcacacccccacaaaattggatgaaaacaataagcatatctaagcaactacgatatctgtatggatcaggccaaagtcccgctaagattctccaatgttttcatggtctgagccccgctcctgttcccatctccactgcccctcggccctgtctgtgccctgcctctcagaggagggggctcagatggtgcggcctgagtgtgcggccggcggcatttgggatacacccgtagggtgggcggggtgtgtcccaggcctaattccatctttccaccatgacagagatgcccttgtgaggctggcctccttggcgcctgtcccc

SEQ ID NO: 47 - RNA, 197 nt, synthetic construct
  source 1..197: mol_type = other RNA
gggttgcagacacatatatttttgaggctgggtgacgagaaaatctagagacatgagggacataaatgggcctggcagcctcggctctttgcggctgctggcaggactgagctgtccgggttctccccacacttccagcacagctgtgctctgtgtcctgcctcggcgctctcgcaaatgaagctgcaggccaagaa

SEQ ID NO: 48 - RNA, 650 nt, synthetic construct
  source 1..650: mol_type = other RNA
gggtgtgcctccacaagtgtgcaagggtatatgtatatatttgcaggcatgtgtgaggcgcactagtaagtacataatttgctggcatgattgcaggtaggcatgagtgtgtatgtgctaacatgtaggctggtgtgtgtaggcatgtgtgaataagcatgtataaatgagcttaagtgtgctgtgtgcacatgtgtacacacagctaatttatctgcagacctatatgtgagcatgtaagagtgaacatatgtgtgtgtgtgtagtatgtaagaatgaggaactcggtatgtgcatgtgtagacaggtacccagcagtgtgtgtatatgcgcatgagtgaggatgtataggcagaagatgtgtgtgtctatgagtgagtttgagtgtgcaggcttgtattaggtgtatgtgaggaagcccttgtgtgtgcaggggtgcacatgtttggccacaggcatgggaggtgtatgttaggagcatgtgtgtttgtaggcagactcatgagcaggtgtgtgcaaatacatatccagctgcactgtgggtgtccacccacaccttgtgttcctcatggcctaccccagcctttcttctccactgggtcccactgttccctggagacagagggctagcatgctgtcatttatctgaa

SEQ ID NO: 49 - RNA, 500 nt, synthetic construct; identical to SEQ ID NO: 6
  source 1..500: mol_type = other RNA

SEQ ID NO: 50 - RNA, 1374 nt, synthetic construct
  source 1..1374: mol_type = other RNA
caactcagctcacatcaccagctcacctctggtagccatagcagcccctgcttcagccccaccgcacccctccagggggcctgcctttccctgacacttttggggtctgcctgggggaggaggggagaaagcaccatgagtgctcactaaaacaactttttccatttttaataaaacgccaaaaatatcacaacccaccaaaaatagatgcctctccccctccagccctagccgagctggtcctaggccccgcctagtgccccacccccacccacagtgctgcactcctcctgcccctgccacgcccaccccctgcccacctctccaggctctgctctgcagcacacccgtgggtgacccctcaccccagaagcagcagtggcagcttgggaaatgtgaggaagggaaggagggagagacgggagggaggagagagaggagaagggaggcaggggaggggcagcagaaccaaggcaaatatttcagctgggctatacccctctccccatccctgttatagaagcttagagagccagccagcaatggaaccttctggttcctgcgccaatcgccaccagtatcaattgtgtgagcttgggtgcgagtgcacgcgtgcgtgagtacggagagtatatatagatctctatctcttagcaaaggtgaatgccagatgtaaatggcgcctctgggcaaaggaggcttgtattttgcacattttataaaaacttgagagaatgagatttctgcttgtatatttctaaaaagaggaaggagcccaaaccatcctctccttaccactcccatccctgtgagccctaccttacccctctgcccctagccaaggagtgtgaatttatagatctaactttcataggcaaaacaaaagcttcgagctgttgcgtgtgtgagtctgttgtgtggatgtgcgtgtgtggtccccagccccagactggattggaaaagtgcatggtgggggcctcggggctgtccccacgctgtccctttgccacaagtctgtggggcaagaggctgcaatattccgtcctgggtgtctgggctgctaacctggcctgctcaggcttcccaccctgtgcggggcacacccccaggaagggaccctggacacggctcccacgtccaggcttaaggtggatgcacttcccgcacctccagtcttctgtgtagcagctttaacccacgtttgtctgtcacgtccagtcccgagacggctgagtgaccccaagaaaggcttccccgacacccagacagaggctgcagggctggggctgggtgagggtggcgggcctgcggggacattctactgtgctaaaaagccactgcagacatagcaataaaaacatgtcattttccaaa

SEQ ID NO: 51 - RNA, 1150 nt, synthetic construct
  source 1..1150: mol_type = other RNA
gggtgtgcctccacaagtgtgcaagggtatatgtatatatttgcaggcatgtgtgaggcgcactagtaagtacataatttgctggcatgattgcaggtaggcatgagtgtgtatgtgctaacatgtaggctggtgtgtgtaggcatgtgtgaataagcatgtataaatgagcttaagtgtgctgtgtgcacatgtgtacacacagctaatttatctgcagacctatatgtgagcatgtaagagtgaacatatgtgtgtgtgtgtagtatgtaagaatgaggaactcggtatgtgcatgtgtagacaggtacccagcagtgtgtgtatatgcgcatgagtgaggatgtataggcagaagatgtgtgtgtctatgagtgagtttgagtgtgcaggcttgtattaggtgtatgtgaggaagcccttgtgtgtgcaggggtgcacatgtttggccacaggcatgggaggtgtatgttaggagcatgtgtgtttgtaggcagactcatgagcaggtgtgtgcaaatacatatccagctgcactgtgggtgtccacccacaccttgtgttcctcatggcctaccccagcctttcttctccactgggtcccactgttccctggagacagagggctagcatgctgtcatttatctgaattctggttcctgcgccaatcgccaccagtatcaattgtgtgagcttgggtgcgagtgcacgcgtgcgtgagtacggagagtatatatagatctctatctcttagcaaaggtgaatgccagatgtaaatggcgcctctgggcaaaggaggcttgtattttgcacattttataaaaacttgagagaatgagatttctgcttgtatatttctaaaaagaggaaggagcccaaaccatcctctccttaccactcccatccctgtgagccctaccttacccctctgcccctagccaaggagtgtgaatttatagatctaactttcataggcaaaacaaaagcttcgagctgttgcgtgtgtgagtctgttgtgtggatgtgcgtgtgtggtccccagccccagactggattggaaaagtgcatggtgggggcctcggggctgtccccacgctgtccctttgccacaagtctgtggggcaagaggctgcaatattccgtcctgggt

SEQ ID NO: 52 - RNA, 500 nt, synthetic construct; identical to SEQ ID NO: 28
  source 1..500: mol_type = other RNA

SEQ ID NO: 53 - RNA, 445 nt, synthetic construct; identical to SEQ ID NO: 29
  source 1..445: mol_type = other RNA

SEQ ID NO: 54 - RNA, 500 nt, synthetic construct; identical to SEQ ID NO: 30
  source 1..500: mol_type = other RNA

SEQ ID NO: 55 - RNA, 351 nt, synthetic construct; identical to SEQ ID NO: 31
  source 1..351: mol_type = other RNA

SEQ ID NO: 56 - RNA, 200 nt, synthetic construct; identical to SEQ ID NO: 32
  source 1..200: mol_type = other RNA

SEQ ID NO: 57 - RNA, 250 nt, synthetic construct; identical to SEQ ID NO: 33
  source 1..250: mol_type = other RNA

SEQ ID NO: 58 - RNA, 250 nt, synthetic construct; identical to SEQ ID NO: 34
  source 1..250: mol_type = other RNA

SEQ ID NO: 59 - RNA, 1000 nt, synthetic construct; identical to SEQ ID NO: 35
  source 1..1000: mol_type = other RNA

SEQ ID NO: 60 - RNA, 641 nt, synthetic construct; identical to SEQ ID NO: 36
  source 1..641: mol_type = other RNA

SEQ ID NO: 61 - RNA, 100 nt, synthetic construct; identical to SEQ ID NO: 37
  source 1..100: mol_type = other RNA

SEQ ID NO: 62 - RNA, 656 nt, synthetic construct; identical to SEQ ID NO: 38
  source 1..656: mol_type = other RNA

SEQ ID NO: 63 - RNA, 1076 nt, synthetic construct; identical to SEQ ID NO: 39
  source 1..1076: mol_type = other RNA

SEQ ID NO: 64 - RNA, 500 nt, synthetic construct; identical to SEQ ID NO: 40
  source 1..500: mol_type = other RNA

SEQ ID NO: 65 - RNA, 1000 nt, synthetic construct; identical to SEQ ID NO: 41
  source 1..1000: mol_type = other RNA

SEQ ID NO: 66 - RNA, 418 nt, synthetic construct; identical to SEQ ID NO: 42
  source 1..418: mol_type = other RNA

SEQ ID NO: 67 - RNA, 500 nt, synthetic construct; identical to SEQ ID NO: 43
  source 1..500: mol_type = other RNA

SEQ ID NO: 68 - RNA, 500 nt, synthetic construct; identical to SEQ ID NO: 44
  source 1..500: mol_type = other RNA

SEQ ID NO: 69 - RNA, 326 nt, synthetic construct; identical to SEQ ID NO: 45
  source 1..326: mol_type = other RNA

SEQ ID NO: 70 - RNA, 1000 nt, synthetic construct; identical to SEQ ID NO: 46
  source 1..1000: mol_type = other RNA

SEQ ID NO: 71 - RNA, 197 nt, synthetic construct; identical to SEQ ID NO: 47
  source 1..197: mol_type = other RNA

SEQ ID NO: 72 - RNA, 650 nt, synthetic construct; identical to SEQ ID NO: 48
  source 1..650: mol_type = other RNA

SEQ ID NO: 73 - RNA, 500 nt, synthetic construct; identical to SEQ ID NO: 6
  source 1..500: mol_type = other RNA

SEQ ID NO: 74 - RNA, 1374 nt, synthetic construct; identical to SEQ ID NO: 50
  source 1..1374: mol_type = other RNA

SEQ ID NO: 75 - RNA, 1150 nt, synthetic construct; identical to SEQ ID NO: 51
  source 1..1150: mol_type = other RNA

SEQ ID NO: 76 - DNA, 10 bp, synthetic construct
  source 1..10: mol_type = other DNA
  misc_feature 1..8: note = RNA
  misc_feature 9: note = DNA
  misc_feature 10: note = RNA
cmawtrtktt

SEQ ID NO: 77-80 - skipped (4 entries)

SEQ ID NO: 81 - RNA, 41 nt, synthetic construct
  source 1..41: mol_type = other RNA
gtgaaytcaagtgcagctcawtrttagcttcagtataaaaa

SEQ ID NO: 82 - RNA, 10 nt, synthetic construct
  source 1..10: mol_type = other RNA
gtgmaytcaw

SEQ ID NO: 83 - RNA, 11 nt, synthetic construct
  source 1..11: mol_type = other RNA
gtgmagytcaw

SEQ ID NO: 84-85 - skipped (2 entries)

SEQ ID NO: 86 - RNA, 13 nt, synthetic construct
  source 1..13: mol_type = other RNA
ctgywrtradgyk

SEQ ID NO: 87-90 - skipped (4 entries)

SEQ ID NO: 91 - RNA, 87 nt, synthetic construct
  source 1..87: mol_type = other RNA
agggtctgagcgcccggcggaaaaccgaagttggaagtgtctcttagcagcgcgcggagaagaacggggagccagcatcccgccacc

SEQ ID NO: 92 - RNA, 96 nt, synthetic construct
  source 1..96: mol_type = other RNA
agggacttcgagagggacttagagaaggcagacgcatcccgaactcgctggaggacaaggctcagctcttgccaggccaaattgagacccgccacc

SEQ ID NO: 93 - RNA, 151 nt, synthetic construct
  source 1..151: mol_type = other RNA
agggtgcctctgtttggcgcttttgtgcgcgcccgggtctgttggtgctcagagtgtggtcaggcggctcggactgagcaggactttccttatcccagttgattgtgcagaatacactgcctgtcgcttgtcttctattcaccccgccacc

SEQ ID NO: 94 - RNA, 127 nt, synthetic construct
  source 1..127: mol_type = other RNA
ttaagctgccttctgcggggcttgccttctggccatgcccttcttctctcccttgcacctgtacctcttggtctttgaataaagcctgagtaggaagaagcctgcatgcctggttctctgcgtctgc

SEQ ID NO: 95 - RNA, 137 nt, synthetic construct
  source 1..137: mol_type = other RNA
caccacgtgccagagccgcccacccggagccgcccgcatgcagcttcacctcccctttccaggcgccactgttgagaagctagagattgtatgagaataaacttgttattatggaagcctggctcccaccccatcta

SEQ ID NO: 96 - RNA, 324 nt, synthetic construct
  source 1..324: mol_type = other RNA
attttaggtatctgccttggtttcagtggggacatctggggcttatggggctgggatgaggagctggatgattctaggaaggcccaagttggagaatgatgtggagagtgtgccaagacactgcttttggcattttattcctttctgtttgctggatgtcaattgacccttttattttctcttacttgtgttttcagatattgttaatcctgccagtctttctcttcaagccagggtgcatcctcagaaacctactcaacacagcactctaggcagccactatcaatcaattgaagttgacactctgcattaaatctatttgcc

SEQ ID NO: 97 - RNA, 282 nt, synthetic construct
  source 1..282: mol_type = other RNA
tacttaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcag

SEQ ID NO: 98-100 - skipped (3 entries)

SEQ ID NO: 101 - RNA, 566 nt, synthetic construct
  source 1..566: mol_type = other RNA
gctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaagaaaacaacaacaacaacaactccaactcacattttaggtatctgccttggtttcagtggggacatctggggcttatggggctgggatgaggagctggatgattctaggaaggcccaagttggagaatgatgtggagagtgtgccaagacactgcttttggcattttattcctttctgtttgctggatgtcaattgacccttttattttctcttacttgtgttttcagatattgttaatcctgccagtctttctcttcaagccagggtgcatcctcagaaacctactcaacacagcactctaggcagccactatcaatcaattgaagttgacactctgcattaaatctatttgcc

SEQ ID NO: 102 - RNA, 535 nt, synthetic construct
  source 1..535: mol_type = other RNA
gctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactccaaaacaacaacaacaacaactccaactcacattttaggtatctgccttggtttcagtggggacatctggggcttatggggctgggatgaggagctggatgattctaggaaggcccaagttggagaatgatgtggagagtgtgccaagacactgcttttggcattttattcctttctgtttgctggatgtcaattgacccttttattttctcttacttgtgttttcagatattgttaatcctgccagtctttctcttcaagccagggtgcatcctcagaaacctactcaacacagcactctaggcagccactatcaatcaattgaagttgacactctgcattaaatctatttgcc

SEQ ID NO: 103 - RNA, 524 nt, synthetic construct
  source 1..524: mol_type = other RNA
gctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaagaaaacaacaacaacaacaactccaactcactacttaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcag

SEQ ID NO: 104 - RNA, 493 nt, synthetic construct
  source 1..493: mol_type = other RNA
gctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactccaaaacaacaacaacaacaactccaactcactacttaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcag

SEQ ID NO: 105 - RNA, 566 nt, synthetic construct
  source 1..566: mol_type = other RNA
attttaggtatctgccttggtttcagtggggacatctggggcttatggggctgggatgaggagctggatgattctaggaaggcccaagttggagaatgatgtggagagtgtgccaagacactgcttttggcattttattcctttctgtttgctggatgtcaattgacccttttattttctcttacttgtgttttcagatattgttaatcctgccagtctttctcttcaagccagggtgcatcctcagaaacctactcaacacagcactctaggcagccactatcaatcaattgaagttgacactctgcattaaatctatttgccaaaacaacaacaacaacaactccaactcacgctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaag

SEQ ID NO: 106 - RNA, 515 nt, synthetic construct
  source 1..515: mol_type = other RNA
attttaggtatctgccttggtttcagtggggacatctggggcttatggggctgggatgaggagctggatgattctaggaaggcccaagttggagaatgatgtggagagtgtgccaagacactgcttttggcattttattcctttctgtttgctggatgtcaattgacccttttattttctcttacttgtgttttcagatattgttaatcctgccagtctttctcttcaagccagggtgcatcctcagaaacctactcaacacagcactctaggcagccactatcaatcaattgaagttgacactctgcattaaatctatttgccaaaacaacaacaacaacaactccaactcacctttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcc

SEQ ID NO: 107 - RNA, 524 nt, synthetic construct
  source 1..524: mol_type = other RNA
tacttaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcagaaaacaacaacaacaacaactccaactcacgctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaag

SEQ ID NO: 108 - RNA, 473 nt, synthetic construct
  source 1..473: mol_type = other RNA
tacttaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcagaaaacaacaacaacaacaactccaactcacctttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcc

SEQ ID NO: 109 - RNA, 165 nt, synthetic construct
  source 1..165: mol_type = other RNA
agggaggcgaccggaaggatctttctagtccagcccctcgctttacccggacgaaagacacgggcctgattcgtcgagtctcactgagccttagtcgtcggcaggtcccaggcgcgaagtttctcggcctggaggagggggtcgcgcgaagtgccagccgccacc

SEQ ID NO: 110 - RNA, 87 nt, synthetic construct; identical to SEQ ID NO: 91
  source 1..87: mol_type = other RNA

SEQ ID NO: 111 - RNA, 96 nt, synthetic construct; identical to SEQ ID NO: 92
  source 1..96: mol_type = other RNA

SEQ ID NO: 112 - RNA, 151 nt, synthetic construct; identical to SEQ ID NO: 93
  source 1..151: mol_type = other RNA

SEQ ID NO: 113 - RNA, 311 nt, synthetic construct; identical to SEQ ID NO: 4
  source 1..311: mol_type = other RNA

SEQ ID NO: 114 - RNA, 212 nt, synthetic construct; identical to SEQ ID NO: 10
  source 1..212: mol_type = other RNA

SEQ ID NO: 115 - RNA, 181 nt, synthetic construct; identical to SEQ ID NO: 14
  source 1..181: mol_type = other RNA

SEQ ID NO: 116 - RNA, 161 nt, synthetic construct
  source 1..161: mol_type = other RNA
ctttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcc

SEQ ID NO: 117 - RNA, 31 nt, synthetic construct
  source 1..31: mol_type = other RNA
tcccaaatcctgccccgttggcacagggcca

SEQ ID NO: 118 - RNA, 137 nt, synthetic construct; identical to SEQ ID NO: 95
  source 1..137: mol_type = other RNA

SEQ ID NO: 119 - RNA, 324 nt, synthetic construct; identical to SEQ ID NO: 96
  source 1..324: mol_type = other RNA

SEQ ID NO: 120 - RNA, 282 nt, synthetic construct; identical to SEQ ID NO: 97
  source 1..282: mol_type = other RNA

SEQ ID NO: 121 - RNA, 282 nt, synthetic construct; identical to SEQ ID NO: 97
  source 1..282: mol_type = other RNA

SEQ ID NO: 122 - RNA, 324 nt, synthetic construct; identical to SEQ ID NO: 96
  source 1..324: mol_type = other RNA

SEQ ID NO: 123 - RNA, 311 nt, synthetic construct; identical to SEQ ID NO: 4
  source 1..311: mol_type = other RNA

SEQ ID NO: 124 - RNA, 332 nt, synthetic construct
  source 1..332: mol_type = other RNA
ggacacatggttaccacctggccctgagtgcagtcacgcgtaccaaaagtaataatggactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaag

SEQ ID NO: 125 - RNA, 332 nt, synthetic construct
  source 1..332: mol_type = other RNA
ggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatcgcgtaccaaaagtaataatgagccaggactgaggggctggccccaggacgctctcaagaaatggggctactgtggtttagacactcctgctgctcacattgatgggtggctattaaag

SEQ ID NO: 126 - RNA, 345 nt, synthetic construct
  source 1..345: mol_type = other RNA
attttaggtatctgccttggtttcagtggggacatctggggcttatggggctgggatgaggagctggatgattctaggaaggcccaagttggagaatgatgtggagagtgtgccaagacgcgtaccaaaagtaataatgcactgcttttggcattttattcctttctgtttgctggatgtcaattgacccttttattttctcttacttgtgttttcagatattgttaatcctgccagtctttctcttcaagccagggtgcatcctcagaaacctactcaacacagcactctaggcagccactatcaatcaattgaagttgacactctgcattaaatctatttgcc

SEQ ID NO: 127 - RNA, 345 nt, synthetic construct
  source 1..345: mol_type = other RNA
attttaggtatctgccttggtttcagtggggacatctggggcttatggggctgggatgaggagctggatgattctaggaaggcccaagttggagaatgatgtggagagtgtgccaagacactgcttttggcattttattcctttctgtttgctggatgtcaattgacccttttattttctcttacttgtgttttcagatattgttaatcctgccagtctttctcttcaagccagggtgcatcctcagaacgcgtaccaaaagtaataatgacctactcaacacagcactctaggcagccactatcaatcaattgaagttgacactctgcattaaatctatttgcc

SEQ ID NO: 128 - RNA, 345 nt, synthetic construct
  source 1..345: mol_type = other RNA
attttaggtatctgccttggtttcagtggggacatctggggcttatggggctgggatgaggagctggatgattctaggaaggcccaagttggagaatgatgtggagagtgtgccaagacactgcttttggcattttattcctttctgtttgctggatgtcaattgacccttttattttctcttacttgtgttttcagatattgttaatcctgccagtctttctcttcaagccagggtgcatcctcagaaacctactcaacacagcactctaggcagccactatcaatcaattgaagttgacactctgcattaaatctcgcgtaccaaaagtaataatgatttgcc

SEQ ID NO: 129 - RNA, 303 nt, synthetic construct
  source 1..303: mol_type = other RNA
tcgcgtaccaaaagtaataatgacttaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcag

SEQ ID NO: 130 - RNA, 303 nt, synthetic construct
  source 1..303: mol_type = other RNA
tacttaataaatatttgctgttgatgatagcaatgaccttgagactgcgcgtaccaaaagtaataatgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcag

SEQ ID NO: 131 - RNA, 303 nt, synthetic construct
  source 1..303: mol_type = other RNA
tacttcgcgtaccaaaagtaataatgaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcag

SEQ ID NO: 132 - RNA, 545 nt, synthetic construct
  source 1..545: mol_type = other RNA
tacttaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcagaaaacaacaacaacaacaactccaactcacgctctttggagcacagagaactttgatttttaagcttctacagatatggtcctagagactggggtcctcagacactcccgctgtatgtggtgagcttggctggaaacttgccatcgcgtaccaaaagtaataatgagccaggactgaggggctggccccaggacgctctcaagaaatggggctactgtggtttagacactcctgctgctcacattgatgggtggctattaaag

SEQ ID NO: 133 - RNA, 545 nt, synthetic construct
  source 1..545: mol_type = other RNA
gctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaagaaaacaacaacaacaacaactccaactcactacttaataaatatttgctgttgatgatagcaatgaccttgagactgcgcgtaccaaaagtaataatgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcag

SEQ ID NO: 134 - RNA, 644 nt, synthetic construct
  source 1..644: mol_type = other RNA
ggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatcgcgtaccaaaagtaataatgagccaggactgaggggctggccccaggacgctctcaagaaatggggctactgtggtttagacactcctgctgctcacattgatgggtggctattaaagaaaacaacaacaacaacaactccaactcactacttaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcag

SEQ ID NO: 135 - RNA, 545 nt, synthetic construct
  source 1..545: mol_type = other RNA
tacttaataaatatttgctgttgatgatagcaatgaccttgagactgcgcgtaccaaaagtaataatgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcagaaaacaacaacaacaacaactccaactcacgctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaag

SEQ ID NO: 136 - RNA, 545 nt, synthetic construct
  source 1..545: mol_type = other RNA
gctctttggagcacagagaactttgatttttaagcttctacagatatggtcctagagactggggtcctcagacactcccgctgtatgtggtgagcttggctggaaacttgccatcgcgtaccaaaagtaataatgagccaggactgaggggctggccccaggacgctctcaagaaatggggctactgtggtttagacactcctgctgctcacattgatgggtggctattaaagaaaacaacaacaacaacaactccaactcactacttaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcag

SEQ ID NO: 137 - RNA, 566 nt, synthetic construct
  source 1..566: mol_type = other RNA
gctctttggagcacagagaactttgatttttaagcttctacagatatggtcctagagactggggtcctcagacactcccgctgtatgtggtgagcttggctggaaacttgccatcgcgtaccaaaagtaataatgagccaggactgaggggctggccccaggacgctctcaagaaatggggctactgtggtttagacactcctgctgctcacattgatgggtggctattaaagaaaacaacaacaacaacaactccaactcactacttaataaatatttgctgttgatgatagcaatgaccttgagactgcgcgtaccaaaagtaataatgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcag

SEQ ID NO: 138 - RNA, 55 nt, synthetic construct; identical to SEQ ID NO: 27
  source 1..55: mol_type = other RNA

SEQ ID NO: 139 - RNA, 96 nt, synthetic construct; identical to SEQ ID NO: 92
  source 1..96: mol_type = other RNA

SEQ ID NO: 140 - RNA, 87 nt, synthetic construct
  source 1..87: mol_type = other RNA
ggcgttgaccgcgaaggacgaggcgtccccgtgacgtcctgctcttccaatgaacttcaggcctgcggagacgacggagaacggaag

SEQ ID NO: 141 - RNA, 217 nt, synthetic construct
  source 1..217: mol_type = other RNA
ggcgttgaccgcgaaggacgaggcgtccccgtgacgtcctgctcttccaatgaacttcaggcctgcggagacgacggagaacggaagttctctgcctgtgtgctggttggttgcgcgttgaggccagccccgcctcccattttccggtctcctcagaagtcgcttagctcttcggtggttgtcccacgtccggaggcctagccgtcgcttacctagg

SEQ ID NO: 142 - RNA, 94 nt, synthetic construct
  source 1..94: mol_type = other RNA
cctataacttggaatgtgggtggaggggttcatagttctccctgagtgagacttgcctgcttctctggcccctggtcctgtcctgttctccagc

SEQ ID NO: 143 - RNA, 94 nt, synthetic construct
  source 1..94: mol_type = other RNA
cctataacttggaatatgggtggaggggttcatagttctccctcagtgagacttgcctgcttctctggcccctggtcctgtcgtgttctccagc

SEQ ID NO: 144 - RNA, 431 nt, synthetic construct
  source 1..431: mol_type = other RNA
gggagactgaggacttggattgcttaaggacaccaggcaggcgcgacagccttcagcctctccctctgcagttttggccggttcctttaacttgccttcatctggggctgtttggtatttccacgtgccttgggcccgcccactgcagcctccatcttggaagcggccgccggcgcctagattgagcatttccacagaaattacagttttgtcctttttgaaaaaatagaactgtatttcagaaaaaagaaactacagttttagcatgcagaaaggaaaagggagaacaagccggatcagaagacgaaaactctgcggaagttctgaatcaagaggagagatggggtctttctgtgttgggcaggctgggctcaaactcctggcctcaagcagtcctcccgccttggcctcccaaatgctgggaatacaga

SEQ ID NO: 145 - RNA, 431 nt, synthetic construct
  source 1..431: mol_type = other RNA
gggagactgaggacttggattgcttaaggacaccaggcaggcgcgacagccttcagcctctccctctgcagttttggccggttcctttaacttgccttcatctggggctgtttggtatttccacgtgccttgggcccgcccactgcagcctccatcttggaagcggccgccggcgcctagattgagcatttccacagaaattacagttttgtcctttttgaaaaaatagaactgtatttcagaaaaaagaaactacagttttagcttgcagaaaggaaaagggagaacaagccggatcagaagacgaaaactctgcggaagttctgaatcaagaggagagatggggtctttctgtgttgggcaggctgggctcaaactcctggcctcaagcagtcctcccgccttggcctcccaaatgctgggaatacaga

SEQ ID NO: 146 - RNA, 94 nt, synthetic construct
  source 1..94: mol_type = other RNA
cctataacttggaatgtgggtggaggggttcatagttctccctgagtgagacttgcctgcttctctggcccctggtcctgtcgtgttctccagc

SEQ ID NO: 147-160 - skipped (14 entries)

SEQ ID NO: 161 - RNA, 10 nt, synthetic construct
  source 1..10: mol_type = other RNA
gtgmabyyaa

SEQ ID NO: 162-170 - skipped (9 entries)

SEQ ID NO: 171 - RNA, 30 nt, synthetic construct
  source 1..30: mol_type = other RNA
aaaacaacaacaacaacaactccaactcac

SEQ ID NO: 172 - protein, 15 aa, synthetic construct
GGGGSGGGGSGGGGS

SEQ ID NO: 173 - protein, 20 aa, synthetic construct
GGSGGSGGSGGSGGSGGSGG

SEQ ID NO: 174 - protein, 48 aa, synthetic construct
HPVGLLARVPLSLYSGHPVGLLARVPLSLYSGLSGRSDNHGGGSGGGS

SEQ ID NO: 175 - protein, 20 aa, synthetic construct
GPPSGSSPMPYDLYHPSGGG

SEQ ID NO: 176 - protein, 20 aa, synthetic construct
METDTLLLWVLLLWVPGSTG

SEQ ID NO: 177 - skipped

SEQ ID NO: 178 - protein, 20 aa, synthetic construct
MYRMQLLSCIALSLALVTNS

SEQ ID NO: 179-180 - skipped (2 entries)

SEQ ID NO: 181 - protein, 242 aa, synthetic construct
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQA

SEQ ID NO: 182 - protein, 312 aa, synthetic construct
NIDVCKLGTVTVQPAPVIPLGSAANISCSLNPKQGCSHYPSSNELILLKFVNDVLVENLHGKKVHDHTGHSSTFQVTNLSLGMTLFVCKLNCSNSQKKPPVPVCGVEISVGVAPEPPQNISCVQEGENGTVACSWNSGKVTYLKTNYTLQLSGPNNLTCQKQCFSDNRQNCNRLDLGINLSPDLAESRFIVRVTAINDLGNSSSLPHTFTFLDIVIPLPPWDIRINFLNASGSRGTLQWEDEGQVVLNQLRYQPLNSTSWNMVNATNAKGKYDLRDLRPFTEYEFQISSKLHLSGGSWSNWSESLRTRTPEE

SEQ ID NO: 183 - protein, 213 aa, synthetic construct
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENP

SEQ ID NO: 184 - protein, 296 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEE

SEQ ID NO: 185 - protein, 188 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQ

SEQ ID NO: 186 - protein, 239 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 187-190 - skipped (4 entries)

SEQ ID NO: 191 - protein, 229 aa, synthetic construct
GPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 192 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 232 aa, synthetic construct
PRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 194 - protein, 115 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 195-200 - skipped (6 entries)

SEQ ID NO: 201 - protein, 313 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 202 - protein, 193 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 203-210 - skipped (8 entries)

SEQ ID NO: 211 - protein, 133 aa, synthetic construct [sequence omitted]

SEQ ID NO: 212-300 - skipped (89 entries)

SEQ ID NO: 301 - protein, 1075 aa, synthetic construct
METDTLLLWVLLLWVPGSTGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHPVGLLARVPLSLYSGHPVGLLARVPLSLYSGLSGRSDNHGGGSGGGSQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGGGGSGGGGSGGGGSGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 302 - protein, 1075 aa, synthetic construct
METDTLLLWVLLLWVPGSTGGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGGGGSGGGGSGGGGSQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAHPVGLLARVPLSLYSGHPVGLLARVPLSLYSGLSGRSDNHGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 303 - protein, 1108 aa, synthetic construct
METDTLLLWVLLLWVPGSTGQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAHPVGLLARVPLSLYSGHPVGLLARVPLSLYSGLSGRSDNHGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHPVGLLARVPLSLYSGHPVGLLARVPLSLYSGLSGRSDNHGGGSGGGSGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 304-310 - skipped (7 entries)

SEQ ID NO: 311 - protein, 492 aa, synthetic construct
METDTLLLWVLLLWVPGSTGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHPVGLLARVPLSLYSGHPVGLLARVPLSLYSGLSGRSDNHGGGSGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTEKQVTLTCMVTDFMPEDIYVEWTNNGKTELNYKNTEPVLDSDGSYFMYSWLRVEKKNWVERNSYSCSVVHEGLHNHHTTKSFSRTPG

SEQ ID NO: 312 - protein, 612 aa, synthetic construct
METDTLLLWVLLLWVPGSTGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSHPVGLLARVPLSLYSGHPVGLLARVPLSLYSGLSGRSDNHGGGSGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPRVYVLPPPEEEMTKKQVTLTCMVTDFMPEDIYVEWTNNGKTELNYKNTEPVLVSDGSYTMYSKLRVEKKNWVERNSYSCSVVHEGLHNHHTTKSFSRTPG

SEQ ID NO: 313 - protein, 782 aa, synthetic construct
METDTLLLWVLLLWVPGSTGQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAHPVGLLARVPLSLYSGHPVGLLARVPLSLYSGLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHPVGLLARVPLSLYSGHPVGLLARVPLSLYSGLSGRSDNHGGGSGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTEKQVTLTCMVTDFMPEDIYVEWTNNGKTELNYKNTEPVLDSDGSYFMYSWLRVEKKNWVERNSYSCSVVHEGLHNHHTTKSFSRTPG

SEQ ID NO: 314 - protein, 972 aa, synthetic construct
METDTLLLWVLLLWVPGSTGNIDVCKLGTVTVQPAPVIPLGSAANISCSLNPKQGCSHYPSSNELILLKFVNDVLVENLHGKKVHDHTGHSSTFQVTNLSLGMTLFVCKLNCSNSQKKPPVPVCGVEISVGVAPEPPQNISCVQEGENGTVACSWNSGKVTYLKTNYTLQLSGPNNLTCQKQCFSDNRQNCNRLDLGINLSPDLAESRFIVRVTAINDLGNSSSLPHTFTFLDIVIPLPPWDIRINFLNASGSRGTLQWEDEGQVVLNQLRYQPLNSTSWNMVNATNAKGKYDLRDLRPFTEYEFQISSKLHLSGGSWSNWSESLRTRTPEEHPVGLLARVPLSLYSGHPVGLLARVPLSLYSGLSGRSDNHGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSHPVGLLARVPLSLYSGHPVGLLARVPLSLYSGLSGRSDNHGGGSGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPRVYVLPPPEEEMTKKQVTLTCMVTDFMPEDIYVEWTNNGKTELNYKNTEPVLVSDGSYTMYSKLRVEKKNWVERNSYSCSVVHEGLHNHHTTKSFSRTPG

SEQ ID NO: 315-320 - skipped (6 entries)

SEQ ID NO: 321 - protein, 495 aa, synthetic construct
MEFGLSWVFLVALFRGVQCRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASHPVGLLARVPLSLYSGHPVGLLARVPLSLYSGLSGRSDNHGGGSGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 322 - protein, 604 aa, synthetic construct
MEFGLSWVFLVALFRGVQCIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSHPVGLLARVPLSLYSGHPVGLLARVPLSLYSGLSGRSDNHGGGSGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 323 - protein, 756 aa, synthetic construct
MEFGLSWVFLVALFRGVQCCRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPHPVGLLARVPLSLYSGHPVGLLARVPLSLYSGLSGRSDNHGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASHPVGLLARVPLSLYSGHPVGLLARVPLSLYSGLSGRSDNHGGGSGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 324 - protein, 948 aa, synthetic construct
MEFGLSWVFLVALFRGVQCKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEHPVGLLARVPLSLYSGHPVGLLARVPLSLYSGLSGRSDNHGGGSGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSHPVGLLARVPLSLYSGHPVGLLARVPLSLYSGLSGRSDNHGGGSGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 325 - protein, 216 aa, synthetic construct
MEFGLSWVFLVALFRGVQCRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 326 - protein, 325 aa, synthetic construct
MEFGLSWVFLVALFRGVQCIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 327 - protein, 477 aa, synthetic construct
MEFGLSWVFLVALFRGVQCCRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPHPVGLLARVPLSLYSGHPVGLLARVPLSLYSGLSGRSDNHGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 328 - protein, 669 aa, synthetic construct
MEFGLSWVFLVALFRGVQCKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEHPVGLLARVPLSLYSGHPVGLLARVPLSLYSGLSGRSDNHGGGSGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 329-330 - skipped (2 entries)

SEQ ID NO: 331 - protein, 1042 aa, synthetic construct
METDTLLLWVLLLWVPGSTGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGGGGSGGGGSGGGGSGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 332 - protein, 541 aa, synthetic construct
METDTLLLWVLLLWVPGSTGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 333 - protein, 544 aa, synthetic construct
MGAMAPRTLLLLLAAALAPTQTRAGPGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 334 - protein, 831 aa, synthetic construct
METDTLLLWVLLLWVPGSTGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHPVGLLARVPLSLYSGHPVGLLARVPLSLYSGLSGRSDNHGGGSGGGSQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQA

SEQ ID NO: 335 - protein, 785 aa, synthetic construct
METDTLLLWVLLLWVPGSTGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 336-340 - skipped (5 entries)

SEQ ID NO: 341 - protein, 459 aa, synthetic construct
METDTLLLWVLLLWVPGSTGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTEKQVTLTCMVTDFMPEDIYVEWTNNGKTELNYKNTEPVLDSDGSYFMYSWLRVEKKNWVERNSYSCSVVHEGLHNHHTTKSFSRTPG

SEQ ID NO: 342 - protein, 579 aa, synthetic construct
METDTLLLWVLLLWVPGSTGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPRVYVLPPPEEEMTKKQVTLTCMVTDFMPEDIYVEWTNNGKTELNYKNTEPVLVSDGSYTMYSKLRVEKKNWVERNSYSCSVVHEGLHNHHTTKSFSRTPG

SEQ ID NO: 343 - protein, 716 aa, synthetic construct
METDTLLLWVLLLWVPGSTGQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTEKQVTLTCMVTDFMPEDIYVEWTNNGKTELNYKNTEPVLDSDGSYFMYSWLRVEKKNWVERNSYSCSVVHEGLHNHHTTKSFSRTPG

SEQ ID NO: 344 - protein, 906 aa, synthetic construct
METDTLLLWVLLLWVPGSTGNIDVCKLGTVTVQPAPVIPLGSAANISCSLNPKQGCSHYPSSNELILLKFVNDVLVENLHGKKVHDHTGHSSTFQVTNLSLGMTLFVCKLNCSNSQKKPPVPVCGVEISVGVAPEPPQNISCVQEGENGTVACSWNSGKVTYLKTNYTLQLSGPNNLTCQKQCFSDNRQNCNRLDLGINLSPDLAESRFIVRVTAINDLGNSSSLPHTFTFLDIVIPLPPWDIRINFLNASGSRGTLQWEDEGQVVLNQLRYQPLNSTSWNMVNATNAKGKYDLRDLRPFTEYEFQISSKLHLSGGSWSNWSESLRTRTPEEGGGGSGGGGSGGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPRVYVLPPPEEEMTKKQVTLTCMVTDFMPEDIYVEWTNNGKTELNYKNTEPVLVSDGSYTMYSKLRVEKKNWVERNSYSCSVVHEGLHNHHTTKSFSRTPG

SEQ ID NO: 345-350 - skipped (6 entries)

SEQ ID NO: 351 - protein, 388 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHPVGLLARVPLSLYSGHPVGLLARVPLSLYSGLSGRSDNHGGGSGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQ

SEQ ID NO: 352 - protein, 518 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHPVGLLARVPLSLYSGHPVGLLARVPLSLYSGLSGRSDNHGGGSGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 353 - protein, 351 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGPPSGSSPMPYDLYHPSGGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYE

SEQ ID NO: 354 - protein, 439 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHPVGLLARVPLSLYSGHPVGLLARVPLSLYSGLSGRSDNHGGGSGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 355 - protein, 569 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHPVGLLARVPLSLYSGHPVGLLARVPLSLYSGLSGRSDNHGGGSGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 356 - protein, 686 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHPVGLLARVPLSLYSGHPVGLLARVPLSLYSGLSGRSDNHGGGSGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 686 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHPVGLLARVPLSLYSGHPVGLLARVPLSLYSGLSGRSDNHGGGSGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 358 - protein, 635 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHPVGLLARVPLSLYSGHPVGLLARVPLSLYSGLSGRSDNHGGGSGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 536 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 360 - protein, 485 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 361 - protein, 635 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHPVGLLARVPLSLYSGHPVGLLARVPLSLYSGLSGRSDNHGGGSGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGGGGSGGGGSGGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 362-370 - skipped (9 entries)

SEQ ID NO: 371 - protein, 152 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 372 - protein, 282 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 373 - protein, 150 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIIS

SEQ ID NO: 374-460 - skipped (87 entries)

SEQ ID NO: 461 - RNA, 2162 nt, synthetic construct
  source 1..2162: mol_type = other RNA
agggtttcgtctttagatcttctggtccccacagactcagagagaacccgccaccatgtgcccccagaagctgaccatcagctggttcgccatcgtgctgctggtgagccctctgatggccatgtgggagctcgagaaggacgtgtacgtggtggaggtggactggacccccgacgcccccggcgagaccgtgaacctgacctgtgacacccccgaggaggacgacatcacctggaccagcgaccagcggcacggcgtgatcggaagcggcaagaccctgaccatcaccgtcaaggagttcctggacgccggccagtacacctgccacaagggcggcgagacactgagccacagccacctgctgttacacaagaaggagaacggcatctggagcaccgagattctgaagaacttcaagaacaagaccttcctgaagtgcgaagcccccaattacagcggccggttcacctgcagctggctggtgcagcggaacatggacctgaagttcaatatcaagtcaagcagcagcagccccgatagccgggccgtgacctgcggcatggccagcctgagcgccgagaaggtgaccctggaccagagggactacgagaagtacagcgtgagctgtcaggaggacgtgacatgcccaaccgccgaggagaccctgcccatcgagctggccctggaggcccggcagcagaataagtacgagaattacagcaccagcttcttcatccgggacatcatcaagcccgacccccctaagaacctgcagatgaagcccctgaagaatagccaggtcgaggtgagctgggagtaccccgatagctggagcaccccccacagctacttcagcctgaagttcttcgtgcggatccagcggaagaaggagaagatgaaggagaccgaggagggctgcaatcagaagggcgcattcctggtggagaagaccagcaccgaggtgcagtgcaagggcggcaacgtgtgcgtgcaggcccaggatcggtactacaacagcagctgcagcaagtgggcctgcgtgccatgccgggtgcggagcggcggcggaggctcaggtggcggcggctcaggcgggggcggcagccgggtgatccccgtgagcggccccgcccggtgccttagccagagccggaatctgctgaagaccaccgacgacatggtgaagaccgcccgggagaagctgaagcactacagctgcaccgccgaggacatcgaccacgaggacatcacccgggaccagaccagcaccctgaagacctgcctgcccctggagctgcacaagaacgagagctgcctggcaacccgggagaccagcagcaccacccggggcagctgcctccccccccagaagaccagcctgatgatgaccctgtgcctggggagcatctacgaggacctgaagatgtaccagaccgagttccaggctataaacgccgccctgcagaaccacaaccaccagcagatcatcctggacaagggcatgctggtggccatcgacgagctgatgcagagcctgaaccacaacggcgagactctgcggcagaagccccctgtgggcgaggccgatccctaccgggtgaagatgaagctgtgcatcctgctgcacgccttcagcacccgggtggtgaccatcaacagggtgatgggctacctgagcagcgctagcggcggaggtagcggaggcggcggctccggcggcggcggcagcggtggaggcggcagcggcggcggtagcctgcaagtggtgatcagcgccatcctggccctggtggtgctgaccgtgatcagcctgatcatcctgatcggcggtggcgggagcggcaagcccatccccaacccactgctgggcctggacagcacctgactaactaattaagctgccttctgcggggcttgccttctggccatgcccttcttctctcccttgcacctgtacctcttggtctttgaataaagcctgagtaggaagaagcctgcatgcctggttctctgcgtctgcgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 462 - RNA, 2348 nt, synthetic construct
  source 1..2348: mol_type = other RNA
agggtttcgtctttagatcttctggtccccacagactcagagagaacccgccaccatgtgcccccagaagctgaccatcagctggttcgccatcgtgctgctggtgagccctctgatggccatgtgggagctcgagaaggacgtgtacgtggtggaggtggactggacccccgacgcccccggcgagaccgtgaacctgacctgtgacacccccgaggaggacgacatcacctggaccagcgaccagcggcacggcgtgatcggaagcggcaagaccctgaccatcaccgtcaaggagttcctggacgccggccagtacacctgccacaagggcggcgagacactgagccacagccacctgctgttacacaagaaggagaacggcatctggagcaccgagattctgaagaacttcaagaacaagaccttcctgaagtgcgaagcccccaattacagcggccggttcacctgcagctggctggtgcagcggaacatggacctgaagttcaatatcaagtcaagcagcagcagccccgatagccgggccgtgacctgcggcatggccagcctgagcgccgagaaggtgaccctggaccagagggactacgagaagtacagcgtgagctgtcaggaggacgtgacatgcccaaccgccgaggagaccctgcccatcgagctggccctggaggcccggcagcagaataagtacgagaattacagcaccagcttcttcatccgggacatcatcaagcccgacccccctaagaacctgcagatgaagcccctgaagaatagccaggtcgaggtgagctgggagtaccccgatagctggagcaccccccacagctacttcagcctgaagttcttcgtgcggatccagcggaagaaggagaagatgaaggagaccgaggagggctgcaatcagaagggcgcattcctggtggagaagaccagcaccgaggtgcagtgcaagggcggcaacgtgtgcgtgcaggcccaggatcggtactacaacagcagctgcagcaagtgggcctgcgtgccatgccgggtgcggagcggcggcggaggctcaggtggcggcggctcaggcgggggcggcagccgggtgatccccgtgagcggccccgcccggtgccttagccagagccggaatctgctgaagaccaccgacgacatggtgaagaccgcccgggagaagctgaagcactacagctgcaccgccgaggacatcgaccacgaggacatcacccgggaccagaccagcaccctgaagacctgcctgcccctggagctgcacaagaacgagagctgcctggcaacccgggagaccagcagcaccacccggggcagctgcctccccccccagaagaccagcctgatgatgaccctgtgcctggggagcatctacgaggacctgaagatgtaccagaccgagttccaggctataaacgccgccctgcagaaccacaaccaccagcagatcatcctggacaagggcatgctggtggccatcgacgagctgatgcagagcctgaaccacaacggcgagactctgcggcagaagccccctgtgggcgaggccgatccctaccgggtgaagatgaagctgtgcatcctgctgcacgccttcagcacccgggtggtgaccatcaacagggtgatgggctacctgagcagcgctagcggcggaggtagcggaggcggcggctccggcggcggcggcagcggtggaggcggcagcggcggcggtagcctgcaagtggtgatcagcgccatcctggccctggtggtgctgaccgtgatcagcctgatcatcctgatcggcggtggcgggagcggcaagcccatccccaacccactgctgggcctggacagcacctgactaactaaaccggtggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaaggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 463 - RNA, 1670 nt, synthetic construct
  source 1..1670: mol_type = other RNA
agggtttcgtctttagatcttctggtccccacagactcagagagaacccgccaccatggctagcccccagctgcggggctacggcgtgcaggccatccctgtgctgctgttactgctgctgctcctgctgctgcccctgcgggtgacccccggcaccacctgccccccccccgtgagcatcgagcacgccgacatccgggtgaagaactacagcgtgaacagccgggagaggtacgtgtgcaatagcggcttcaagcggaaggccggcaccagcaccctgatcgagtgcgtgatcaacaagaacaccaacgtggcccactggaccacccccagcctgaagtgcatccgggaccccagcttagcccactacagccccgtgcccaccgtggtgaccccaaaggtgaccagccagcccgagagccccagccccagcgccaaggagcctgaggccttctccccaaagagcgacaccgccatgaccaccgagaccgccatcatgcccggcagccggctgacccccagccagaccaccagcgccggcaccacaggcaccggcagccacaagagcagccgggcacccagcctggccgccaccatgaccctggagcctaccgctagcaccagcctgcggatcaccgagatcagcccccacagcagcaagatgaccaaggtggccatcagcaccagcgtgctgctggtgggcgccggcgtggtgatggctttcctggcctggtacatcaagagccggcagcccagccagccttgccgggtggaggtggagaccatggagactgtgcccatgaccgtgcgggccagcagcaaggaggacgaggacaccggcgccgggggcagcggcggtagcggaggcagcggcggcagcggcggaagcggcggctccggcggcatgaagatcctgaagccctacatgcggaacaccagcatcagctgctacctgtgcttcctgctgaacagccacttcctgaccgaggccggcatccacgtgttcatcctgggctgcgtgagcgtgggcctgcctaagaccgaggccaactggatcgacgtgcggtacgacctggagaagatcgagagcctgatccagagcatccacatcgacaccaccctgtacaccgacagcgacttccaccccagctgcaaggtgaccgccatgaactgctttctgctggagctgcaggtgatcctgcacgagtacagcaacatgaccctgaacgagaccgtgcggaacgtgctgtacctggccaacagcaccctgagcagcaacaagaacgtggccgagagcggctgcaaggagtgcgaggagctggaggagaagaccttcaccgagttcctgcagagcttcatccggatcgtgcagatgttcatcaacaccagctgactaactaattaagctgccttctgcggggcttgccttctggccatgcccttcttctctcccttgcacctgtacctcttggtctttgaataaagcctgagtaggaagaagcctgcatgcctggttctctgcgtctgcgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 464 - RNA, 1856 nt, synthetic construct
  source 1..1856: mol_type = other RNA
agggtttcgtctttagatcttctggtccccacagactcagagagaacccgccaccatggctagcccccagctgcggggctacggcgtgcaggccatccctgtgctgctgttactgctgctgctcctgctgctgcccctgcgggtgacccccggcaccacctgccccccccccgtgagcatcgagcacgccgacatccgggtgaagaactacagcgtgaacagccgggagaggtacgtgtgcaatagcggcttcaagcggaaggccggcaccagcaccctgatcgagtgcgtgatcaacaagaacaccaacgtggcccactggaccacccccagcctgaagtgcatccgggaccccagcttagcccactacagccccgtgcccaccgtggtgaccccaaaggtgaccagccagcccgagagccccagccccagcgccaaggagcctgaggccttctccccaaagagcgacaccgccatgaccaccgagaccgccatcatgcccggcagccggctgacccccagccagaccaccagcgccggcaccacaggcaccggcagccacaagagcagccgggcacccagcctggccgccaccatgaccctggagcctaccgctagcaccagcctgcggatcaccgagatcagcccccacagcagcaagatgaccaaggtggccatcagcaccagcgtgctgctggtgggcgccggcgtggtgatggctttcctggcctggtacatcaagagccggcagcccagccagccttgccgggtggaggtggagaccatggagactgtgcccatgaccgtgcgggccagcagcaaggaggacgaggacaccggcgccgggggcagcggcggtagcggaggcagcggcggcagcggcggaagcggcggctccggcggcatgaagatcctgaagccctacatgcggaacaccagcatcagctgctacctgtgcttcctgctgaacagccacttcctgaccgaggccggcatccacgtgttcatcctgggctgcgtgagcgtgggcctgcctaagaccgaggccaactggatcgacgtgcggtacgacctggagaagatcgagagcctgatccagagcatccacatcgacaccaccctgtacaccgacagcgacttccaccccagctgcaaggtgaccgccatgaactgctttctgctggagctgcaggtgatcctgcacgagtacagcaacatgaccctgaacgagaccgtgcggaacgtgctgtacctggccaacagcaccctgagcagcaacaagaacgtggccgagagcggctgcaaggagtgcgaggagctggaggagaagaccttcaccgagttcctgcagagcttcatccggatcgtgcagatgttcatcaacaccagctgactaactaaaccggtggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaaggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 465 - RNA, 893 nt, synthetic construct
  source 1..893: mol_type = other RNA
agggtttcgtctttagatcttctggtccccacagactcagagagaacccgccaccatggctcggctgtgcgccttcctgatgatcctggtgatgatgagctactactggagcgcatgcagcctgggctgcgacctgccacacacctacaacctgggcaataagcgggctctgaccgtgctggaggagatgcggcggctgccccccctgagctgcctgaaggaccggaaggacttcggcttccccctggagaaggtggacaaccagcagatccaaaaggcacaggccatcctggtgctgcgggacctgacccagcagatcctgaacctgttcaccagcaaggacctgagcgccacctggaacgccaccctgctggacagcttctgcaacgacctgcaccagcagctgaacgacctgaaggcctgcgtcatgcaggagccaccactgacccaggaggacagcctgttagctgtgcggacttacttccaccggatcaccgtgtacctgcggaagaagaagcacagcctgtgcgcttgggaggtgatccgggccgaggtgtggcgggccctgagcagcagcaccaacctgctggcccggctgagcgaggagaaggagtgactaactaattaagctgccttctgcggggcttgccttctggccatgcccttcttctctcccttgcacctgtacctcttggtctttgaataaagcctgagtaggaagaagcctgcatgcctggttctctgcgtctgcgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 466 - RNA, 1079 nt, synthetic construct
  source 1..1079: mol_type = other RNA
agggtttcgtctttagatcttctggtccccacagactcagagagaacccgccaccatggctcggctgtgcgccttcctgatgatcctggtgatgatgagctactactggagcgcatgcagcctgggctgcgacctgccacacacctacaacctgggcaataagcgggctctgaccgtgctggaggagatgcggcggctgccccccctgagctgcctgaaggaccggaaggacttcggcttccccctggagaaggtggacaaccagcagatccaaaaggcacaggccatcctggtgctgcgggacctgacccagcagatcctgaacctgttcaccagcaaggacctgagcgccacctggaacgccaccctgctggacagcttctgcaacgacctgcaccagcagctgaacgacctgaaggcctgcgtcatgcaggagccaccactgacccaggaggacagcctgttagctgtgcggacttacttccaccggatcaccgtgtacctgcggaagaagaagcacagcctgtgcgcttgggaggtgatccgggccgaggtgtggcgggccctgagcagcagcaccaacctgctggcccggctgagcgaggagaaggagtgactaactaaaccggtggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaaggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 467 - RNA, 615 nt, synthetic construct
  source 1..615: mol_type = other RNA
agggtttcgtctttagatcttctggtccccacagactcagagagaacccgccaccatgtaccggatgcagctgctgagctgcatcgccctgagcctggccctggtgaccaacagcgcccccaccagcagcagcaccaagaagacccagctgcaactggagcacctgctgctggatctgcagatgatcctgaacggcatcaacaattacaagaatcccaagctgacccggatgctgaccttcaagttctacatgcccaagaaggctaccgagctgaagcacctgcagtgcctggaggaggagctgaagcccctggaggaggtgctgaacctggcccagagcaagaacttccacttcgacccccgggacgtggtgagcaacatcaacgtgttcgtgctggagctgaagggcagcgagaccaccttcatgtgcgagtacgccgacgagaccgccaccatcgtggagttcctgaaccggtggatcaccttctgccagagcatcatcagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 468 - RNA, 971 nt, synthetic construct
  source 1..971: mol_type = other RNA
agggtttcgtctttagatcttctggtccccacagactcagagagaacccgccaccatgtaccggatgcagctgctgagctgcatcgccctgagcctggccctggtgaccaacagcgcccccaccagcagcagcaccaagaagacccagctgcaactggagcacctgctgctggatctgcagatgatcctgaacggcatcaacaattacaagaatcccaagctgacccggatgctgaccttcaagttctacatgcccaagaaggctaccgagctgaagcacctgcagtgcctggaggaggagctgaagcccctggaggaggtgctgaacctggcccagagcaagaacttccacttcgacccccgggacgtggtgagcaacatcaacgtgttcgtgctggagctgaagggcagcgagaccaccttcatgtgcgagtacgccgacgagaccgccaccatcgtggagttcctgaaccggtggatcaccttctgccagagcatcatcagctgactaactaaaccggtggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaaggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 469 - RNA, 1007 nt, synthetic construct
  source 1..1007: mol_type = other RNA
agggtttcgtctttagatcttctggtccccacagactcagagagaacccgccaccatgagcgtgctgacccaggtgctggctctgctgctgctgtggctgaccggcgcccggtgcaccacatgccccccccccgtgagcatcgagcacgccgacatccgggtgaagaactacagcgtgaacagccgggagcggtacgtgtgcaacagcggcttcaagcggaaggccgggaccagcaccctgatcgagtgcgtgatcaacaagaacaccaacgtggcccactggaccacccccagcctgaagtgcatccgggacccaagcctggctggcggctcaggcggtagcgggggtagcggcggcagcggcggctccggaggcagcggcggaaattggatcgacgtgcggtacgacctggagaagatcgagagcctgatccagagcatccacatcgacaccaccctgtacaccgacagcgacttccaccctagctgcaaggtgaccgccatgaactgcttcctgctggagctgcaggtgatcctgcacgagtacagcaacatgaccctgaacgagacagtgcggaacgtgctgtacctggccaacagcaccctgagcagcaacaagaacgtggccgagagcggctgcaaggagtgcgaggagctggaggagaagaccttcaccgagttcctgcagtccttcatccggatcgtgcagatgttcatcaacaccagctgactaactaattaagctgccttctgcggggcttgccttctggccatgcccttcttctctcccttgcacctgtacctcttggtctttgaataaagcctgagtaggaagaagcctgcatgcctggttctctgcgtctgcgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 470 - RNA, 1193 nt, synthetic construct
  source 1..1193: mol_type = other RNA
agggtttcgtctttagatcttctggtccccacagactcagagagaacccgccaccatgagcgtgctgacccaggtgctggctctgctgctgctgtggctgaccggcgcccggtgcaccacatgccccccccccgtgagcatcgagcacgccgacatccgggtgaagaactacagcgtgaacagccgggagcggtacgtgtgcaacagcggcttcaagcggaaggccgggaccagcaccctgatcgagtgcgtgatcaacaagaacaccaacgtggcccactggaccacccccagcctgaagtgcatccgggacccaagcctggctggcggctcaggcggtagcgggggtagcggcggcagcggcggctccggaggcagcggcggaaattggatcgacgtgcggtacgacctggagaagatcgagagcctgatccagagcatccacatcgacaccaccctgtacaccgacagcgacttccaccctagctgcaaggtgaccgccatgaactgcttcctgctggagctgcaggtgatcctgcacgagtacagcaacatgaccctgaacgagacagtgcggaacgtgctgtacctggccaacagcaccctgagcagcaacaagaacgtggccgagagcggctgcaaggagtgcgaggagctggaggagaagaccttcaccgagttcctgcagtccttcatccggatcgtgcagatgttcatcaacaccagctgactaactaaaccggtggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaaggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 471 - RNA, 1457 nt, synthetic construct
  source 1..1457: mol_type = other RNA
agggtttcgtctttagatcttctggtccccacagactcagagagaacccgccaccgacgacgacatcgccgccctggtggtggataacggcagcggcatgtgcaaggccggcttcgccggcgacgatgccccccgggccgtgttccccagcatcgtcggacgtccccggcaccagggcgtgatggtgggcatgggccagaaggactcctacgtgggcgacgaggcccagagcaagcggggcatcctgaccctgaagtacccaatcgagcacggcatcgtgaccaactgggacgacatggagaagatctggcaccacaccttctacaatgagctgcgggtggccccagaggagcaccccgtgctgctgaccgaggcccccctgaaccccaaggccaaccgggagaagatgactcagatcatgttcgagacctttaacacccccgccatgtacgtggccattcaggccgtgctgagcctgtacgcctcaggccggaccaccggcatcgtgatggacagcggcgacggcgtgacccacaccgtgcccatctacgagggctacgccctgccccacgccatcctgcggctggacctggccggccgggacctgaccgactacctgatgaagatcctgaccgagcggggctacagcttcaccaccaccgccgagcgggagatcgtgcgggacatcaaggagaagctgtgctacgtggccctggacttcgagcaggagatggccaccgccgccagcagcagcagcctggagaagagctacgagctgcccgacggacaggtgatcaccatcggcaacgagcggttccgttgccccgaggccctgttccagccctcattcctgggcatggagagctgcggcatccacgagaccaccttcaacagcatcatgaagtgcgacgtggacatccggaaggatctgtacgccaacaccgtgctgagcggcggcaccaccatgtaccccggcattgccgatcggatgcagaaggagatcaccgcgctggccccaagcaccatgaagatcaagatcatcgctccccccgagcggaagtacagcgtgtggatcggcggcagcatactggctagcctgagcaccttccagcagatgtggatcagcaagcaggagtacgacgagagcggccctagcatcgtgcaccggaaatgcttctgactaactaattaagctgccttctgcggggcttgccttctggccatgcccttcttctctcccttgcacctgtacctcttggtctttgaataaagcctgagtaggaagaagcctgcatgcctggttctctgcgtctgcgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 472 - RNA, 1643 nt, synthetic construct
  source 1..1643: mol_type = other RNA
agggtttcgtctttagatcttctggtccccacagactcagagagaacccgccaccgacgacgacatcgccgccctggtggtggataacggcagcggcatgtgcaaggccggcttcgccggcgacgatgccccccgggccgtgttccccagcatcgtcggacgtccccggcaccagggcgtgatggtgggcatgggccagaaggactcctacgtgggcgacgaggcccagagcaagcggggcatcctgaccctgaagtacccaatcgagcacggcatcgtgaccaactgggacgacatggagaagatctggcaccacaccttctacaatgagctgcgggtggccccagaggagcaccccgtgctgctgaccgaggcccccctgaaccccaaggccaaccgggagaagatgactcagatcatgttcgagacctttaacacccccgccatgtacgtggccattcaggccgtgctgagcctgtacgcctcaggccggaccaccggcatcgtgatggacagcggcgacggcgtgacccacaccgtgcccatctacgagggctacgccctgccccacgccatcctgcggctggacctggccggccgggacctgaccgactacctgatgaagatcctgaccgagcggggctacagcttcaccaccaccgccgagcgggagatcgtgcgggacatcaaggagaagctgtgctacgtggccctggacttcgagcaggagatggccaccgccgccagcagcagcagcctggagaagagctacgagctgcccgacggacaggtgatcaccatcggcaacgagcggttccgttgccccgaggccctgttccagccctcattcctgggcatggagagctgcggcatccacgagaccaccttcaacagcatcatgaagtgcgacgtggacatccggaaggatctgtacgccaacaccgtgctgagcggcggcaccaccatgtaccccggcattgccgatcggatgcagaaggagatcaccgcgctggccccaagcaccatgaagatcaagatcatcgctccccccgagcggaagtacagcgtgtggatcggcggcagcatactggctagcctgagcaccttccagcagatgtggatcagcaagcaggagtacgacgagagcggccctagcatcgtgcaccggaaatgcttctgactaactaaaccggtggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaaggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 473 - RNA, 3560 nt, synthetic construct
  source 1..3560: mol_type = other RNA
agggtttcgtctttagatcttctggtccccacagactcagagagaacccgccaccatggagaccgacaccctgctgctgtgggtgctgctgctatgggtgcccggcagcaccggcatgtgggagctggagaaggacgtgtacgtggtggaggtggactggaccccagacgctcccggcgagaccgtgaacctgacctgcgacacccccgaggaggacgacatcacttggaccagcgaccagcggcacggcgtgatcggcagcggcaagaccctgactatcaccgtgaaggagttcctggacgccggccagtacacctgccacaagggcggcgagaccctgagccacagccacctgctgctacacaagaaggagaacggcatctggagcaccgagatcctgaagaacttcaagaacaagaccttcctgaagtgcgaggcccccaactacagcggccggttcacatgcagctggctggtgcagaggaacatggacctgaagttcaacatcaagtcaagcagcagcagccccgacagccgggccgtgacctgcggcatggccagcctgagcgccgagaaggtgaccctggaccagcgggactacgagaagtacagcgtgagctgccaggaggacgtcacctgccccacagccgaggagaccctgcccatcgagctggccctggaggcccggcagcagaacaagtacgagaactacagcaccagcttcttcatccgggacatcatcaagcccgacccacccaagaacctgcagatgaagcccctgaagaacagccaggtggaggtgagctgggagtacccagacagctggagcaccccccacagctacttcagcctgaagttcttcgtgcggatccagcggaagaaggagaagatgaaggagaccgaggagggctgcaaccagaagggcgccttcctggtggagaagactagcaccgaggtgcagtgcaagggcggcaacgtgtgcgtgcaggcacaggatcggtactacaacagcagctgcagcaagtgggcctgcgtgccctgccgggtgcggagcggcggcgggggcagcggagggggcggcagcggcgggggcggtagccgggtgatacccgtgagcggcccagcacggtgcctgagccagagcaggaacctgctgaagaccaccgacgacatggtgaagaccgcccgggagaagctgaagcactacagctgcaccgccgaggacattgaccacgaggacatcacccgggaccagaccagcaccctgaagacctgcctgcccctggagctgcacaagaacgagagctgtctggccacccgggagaccagcagcaccaccaggggcagctgcttaccaccccagaagaccagcctgatgatgaccctgtgcctgggcagcatctacgaggacctgaagatgtaccagaccgagttccaggccatcaacgcagccctgcagaaccacaaccaccagcagatcatcctggacaagggcatgctggtggctatcgacgagctgatgcagagcctgaaccacaacggcgagacactgcggcagaagccccccgtgggcgaggccgacccctaccgggtgaagatgaagctgtgcatcctgctacacgccttcagcacccgggtggtgaccatcaaccgggtgatgggctacctgagcagcgcccaccccgtgggcctgctcgcccgggtgcctctgagcctgtacagcggccaccccgtgggcctgctggcccgggtgcccctgagcctatacagcggcctgagcggccggagcgacaaccacggcggcggatcaggcggcggaagccagctgggcgctagcggccccggcgatggctgctgtgtggagaagaccagcttccccgagggcgccagcggcagccccctgggccctaggaacctgagctgctaccgggtgagcaagaccgactacgagtgcagctggcagtacgacggccccgaggacaacgtgagccacgtgctgtggtgctgcttcgtgccccccaaccacacccacaccggccaggagcggtgccggtacttcagcagcggccccgaccggaccgtgcagttctgggagcaggacggcatccccgtgctgagcaaggtgaacttctgggtggagagcaggctgggcaaccggaccatgaagagccagaagatcagccagtacctgtacaactggaccaagacaaccccccccctgggccacatcaaggtgagccagagccaccggcagctgcggatggactggaacgtgtcagaggaggccggcgcagaggtgcagttccggcggaggatgcccaccaccaattggaccctgggcgactgcggcccccaggtgaacagcggcagcggagtgctgggcgatatcaggggcagcatgagcgagagctgcctgtgccccagcgagaacatggctcaggagatccagatccggcggcggaggcggctgagcagcggcgcccccggcggcccctggagcgactggagcatgcctgtgtgcgtgccaccagaggtgctgcctcaggccggcggcgggggaagcggcggtggcggcagcggcggaggaggcagcggccccaccatcaagccctgccccccttgcaagtgccctgccccaaacgcagccggcggccccagcgtgttcatcttccccccaaagatcaaggacgtgctgatgatcagcctgagccccatcgtgacctgcgtggtggtggacgtgagcgaggacgaccccgacgtgcagatcagctggttcgtgaacaacgtggaggtgcacaccgcccagacccagacccaccgggaggattacaacagcacccttcgggtggtgagcgccctgcccatccagcaccaggactggatgagcggcaaggagttcaagtgcaaggtgaacaacaaggacctgggcgcacccatagagcggaccatctcaaagcccaagggcagcgtgcgggccccccaggtgtacgtgctgcctccacccgaggaggagatgaccaagaagcaggtgaccctgacctgcatggtgaccgacttcatgcccgaggacatctacgtggagtggaccaacaacggcaagaccgagcttaactacaagaacaccgagcccgtgctggacagcgacggctcatacttcatgtacagcaagctgcgggtggagaagaagaactgggtggagcggaacagctacagctgcagcgtggtgcacgagggcctgcacaaccaccacactaccaagagcttcagccggacccccggctgactaactaattaagctgccttctgcggggcttgccttctggccatgcccttcttctctcccttgcacctgtacctcttggtctttgaataaagcctgagtaggaagaagcctgcatgcctggttctctgcgtctgcgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 474 - RNA, 3746 nt, synthetic construct
  source 1..3746: mol_type = other RNA
agggtttcgtctttagatcttctggtccccacagactcagagagaacccgccaccatggagaccgacaccctgctgctgtgggtgctgctgctatgggtgcccggcagcaccggcatgtgggagctggagaaggacgtgtacgtggtggaggtggactggaccccagacgctcccggcgagaccgtgaacctgacctgcgacacccccgaggaggacgacatcacttggaccagcgaccagcggcacggcgtgatcggcagcggcaagaccctgactatcaccgtgaaggagttcctggacgccggccagtacacctgccacaagggcggcgagaccctgagccacagccacctgctgctacacaagaaggagaacggcatctggagcaccgagatcctgaagaacttcaagaacaagaccttcctgaagtgcgaggcccccaactacagcggccggttcacatgcagctggctggtgcagaggaacatggacctgaagttcaacatcaagtcaagcagcagcagccccgacagccgggccgtgacctgcggcatggccagcctgagcgccgagaaggtgaccctggaccagcgggactacgagaagtacagcgtgagctgccaggaggacgtcacctgccccacagccgaggagaccctgcccatcgagctggccctggaggcccggcagcagaacaagtacgagaactacagcaccagcttcttcatccgggacatcatcaagcccgacccacccaagaacctgcagatgaagcccctgaagaacagccaggtggaggtgagctgggagtacccagacagctggagcaccccccacagctacttcagcctgaagttcttcgtgcggatccagcggaagaaggagaagatgaaggagaccgaggagggctgcaaccagaagggcgccttcctggtggagaagactagcaccgaggtgcagtgcaagggcggcaacgtgtgcgtgcaggcacaggatcggtactacaacagcagctgcagcaagtgggcctgcgtgccctgccgggtgcggagcggcggcgggggcagcggagggggcggcagcggcgggggcggtagccgggtgatacccgtgagcggcccagcacggtgcctgagccagagcaggaacctgctgaagaccaccgacgacatggtgaagaccgcccgggagaagctgaagcactacagctgcaccgccgaggacattgaccacgaggacatcacccgggaccagaccagcaccctgaagacctgcctgcccctggagctgcacaagaacgagagctgtctggccacccgggagaccagcagcaccaccaggggcagctgcttaccaccccagaagaccagcctgatgatgaccctgtgcctgggcagcatctacgaggacctgaagatgtaccagaccgagttccaggccatcaacgcagccctgcagaaccacaaccaccagcagatcatcctggacaagggcatgctggtggctatcgacgagctgatgcagagcctgaaccacaacggcgagacactgcggcagaagccccccgtgggcgaggccgacccctaccgggtgaagatgaagctgtgcatcctgctacacgccttcagcacccgggtggtgaccatcaaccgggtgatgggctacctgagcagcgcccaccccgtgggcctgctcgcccgggtgcctctgagcctgtacagcggccaccccgtgggcctgctggcccgggtgcccctgagcctatacagcggcctgagcggccggagcgacaaccacggcggcggatcaggcggcggaagccagctgggcgctagcggccccggcgatggctgctgtgtggagaagaccagcttccccgagggcgccagcggcagccccctgggccctaggaacctgagctgctaccgggtgagcaagaccgactacgagtgcagctggcagtacgacggccccgaggacaacgtgagccacgtgctgtggtgctgcttcgtgccccccaaccacacccacaccggccaggagcggtgccggtacttcagcagcggccccgaccggaccgtgcagttctgggagcaggacggcatccccgtgctgagcaaggtgaacttctgggtggagagcaggctgggcaaccggaccatgaagagccagaagatcagccagtacctgtacaactggaccaagacaaccccccccctgggccacatcaaggtgagccagagccaccggcagctgcggatggactggaacgtgtcagaggaggccggcgcagaggtgcagttccggcggaggatgcccaccaccaattggaccctgggcgactgcggcccccaggtgaacagcggcagcggagtgctgggcgatatcaggggcagcatgagcgagagctgcctgtgccccagcgagaacatggctcaggagatccagatccggcggcggaggcggctgagcagcggcgcccccggcggcccctggagcgactggagcatgcctgtgtgcgtgccaccagaggtgctgcctcaggccggcggcgggggaagcggcggtggcggcagcggcggaggaggcagcggccccaccatcaagccctgccccccttgcaagtgccctgccccaaacgcagccggcggccccagcgtgttcatcttccccccaaagatcaaggacgtgctgatgatcagcctgagccccatcgtgacctgcgtggtggtggacgtgagcgaggacgaccccgacgtgcagatcagctggttcgtgaacaacgtggaggtgcacaccgcccagacccagacccaccgggaggattacaacagcacccttcgggtggtgagcgccctgcccatccagcaccaggactggatgagcggcaaggagttcaagtgcaaggtgaacaacaaggacctgggcgcacccatagagcggaccatctcaaagcccaagggcagcgtgcgggccccccaggtgtacgtgctgcctccacccgaggaggagatgaccaagaagcaggtgaccctgacctgcatggtgaccgacttcatgcccgaggacatctacgtggagtggaccaacaacggcaagaccgagcttaactacaagaacaccgagcccgtgctggacagcgacggctcatacttcatgtacagcaagctgcgggtggagaagaagaactgggtggagcggaacagctacagctgcagcgtggtgcacgagggcctgcacaaccaccacactaccaagagcttcagccggacccccggctgactaactaaaccggtggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaaggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 475 - RNA, 1958 nt, synthetic construct
  source 1..1958: mol_type = other RNA
agggtttcgtctttagatcttctggtccccacagactcagagagaacccgccaccatggagaccgacaccctgctgctgtgggtgctgctgctttgggtgcccggcagcaccggcatgtgggagctggagaaggacgtgtacgtggtggaggtggactggacccccgacgcccccggcgagaccgtgaacctgacctgcgacacccccgaggaggacgatatcacatggaccagcgaccagcggcacggcgtcatcggcagcggcaagactctgaccatcaccgtgaaggagttcctggacgccggccagtacacctgccacaagggcggcgagaccctgagccacagccacctgctgctccacaagaaggagaacggcatctggagcaccgagatcctgaagaacttcaagaataagaccttcctgaagtgcgaggcccccaactacagcggccggttcacctgcagctggctggtgcagcggaacatggacctgaagttcaacatcaagagcagctcaagcagccccgacagccgggccgtgacctgcggcatggccagcctgagcgccgagaaggtgaccctggaccagcgggactacgagaagtactcagtgagctgccaggaggacgtgacctgccccacagccgaggagaccctgccaatcgagctggccctggaggcccggcagcagaacaagtacgagaactacagcaccagctttttcatccgggacattatcaagcctgacccccccaagaacctgcagatgaagcccctgaagaatagccaggtggaggtgagctgggagtaccccgacagctggagcaccccccacagctacttcagcctgaagttcttcgtgcggatccagcggaagaaggagaagatgaaggagaccgaggagggctgtaaccagaagggcgccttcctggtggagaagaccagcaccgaggtgcagtgcaagggcggcaacgtgtgcgtgcaggcccaggaccggtactacaacagcagctgcagcaagtgggcctgcgtgccctgccgggtgcggagcggcggcggaggaagcggcggcggcggcagcggcggaggcggcagccgggtgattcccgtgagcggccccgcccggtgcctgagccagagccggaacctgctgaagacaaccgacgacatggtgaagaccgcccgggagaagctgaagcactacagctgcaccgccgaggacatcgaccacgaggacatcacccgggaccagaccagcaccctgaagacctgcctgcccctggagctgcacaagaacgagagctgcctggccacccgggagaccagctcaaccacccggggcagctgtctgcccccacagaagacctcccttatgatgaccctgtgcctgggcagcatctacgaggacctgaagatgtaccagaccgagttccaggccatcaacgccgcactgcagaaccacaaccaccagcagatcatcctggacaagggcatgctggtggccatcgacgagctgatgcagagcctgaaccacaacggagagaccctgcggcagaagccccccgtgggcgaggccgacccataccgggtgaagatgaagctgtgcatcctgctgcacgccttcagcacccgggtggtgaccatcaaccgggtgatgggctacctgagcagcgcctgactaactaattaagctgccttctgcggggcttgccttctggccatgcccttcttctctcccttgcacctgtacctcttggtctttgaataaagcctgagtaggaagaagcctgcatgcctggttctctgcgtctgcgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 476 - RNA, 2144 nt, synthetic construct
  source 1..2144: mol_type = other RNA
agggtttcgtctttagatcttctggtccccacagactcagagagaacccgccaccatggagaccgacaccctgctgctgtgggtgctgctgctttgggtgcccggcagcaccggcatgtgggagctggagaaggacgtgtacgtggtggaggtggactggacccccgacgcccccggcgagaccgtgaacctgacctgcgacacccccgaggaggacgatatcacatggaccagcgaccagcggcacggcgtcatcggcagcggcaagactctgaccatcaccgtgaaggagttcctggacgccggccagtacacctgccacaagggcggcgagaccctgagccacagccacctgctgctccacaagaaggagaacggcatctggagcaccgagatcctgaagaacttcaagaataagaccttcctgaagtgcgaggcccccaactacagcggccggttcacctgcagctggctggtgcagcggaacatggacctgaagttcaacatcaagagcagctcaagcagccccgacagccgggccgtgacctgcggcatggccagcctgagcgccgagaaggtgaccctggaccagcgggactacgagaagtactcagtgagctgccaggaggacgtgacctgccccacagccgaggagaccctgccaatcgagctggccctggaggcccggcagcagaacaagtacgagaactacagcaccagctttttcatccgggacattatcaagcctgacccccccaagaacctgcagatgaagcccctgaagaatagccaggtggaggtgagctgggagtaccccgacagctggagcaccccccacagctacttcagcctgaagttcttcgtgcggatccagcggaagaaggagaagatgaaggagaccgaggagggctgtaaccagaagggcgccttcctggtggagaagaccagcaccgaggtgcagtgcaagggcggcaacgtgtgcgtgcaggcccaggaccggtactacaacagcagctgcagcaagtgggcctgcgtgccctgccgggtgcggagcggcggcggaggaagcggcggcggcggcagcggcggaggcggcagccgggtgattcccgtgagcggccccgcccggtgcctgagccagagccggaacctgctgaagacaaccgacgacatggtgaagaccgcccgggagaagctgaagcactacagctgcaccgccgaggacatcgaccacgaggacatcacccgggaccagaccagcaccctgaagacctgcctgcccctggagctgcacaagaacgagagctgcctggccacccgggagaccagctcaaccacccggggcagctgtctgcccccacagaagacctcccttatgatgaccctgtgcctgggcagcatctacgaggacctgaagatgtaccagaccgagttccaggccatcaacgccgcactgcagaaccacaaccaccagcagatcatcctggacaagggcatgctggtggccatcgacgagctgatgcagagcctgaaccacaacggagagaccctgcggcagaagccccccgtgggcgaggccgacccataccgggtgaagatgaagctgtgcatcctgctgcacgccttcagcacccgggtggtgaccatcaaccgggtgatgggctacctgagcagcgcctgactaactaaaccggtggacacatggttaccacctggccctgagtgcagtcagactgggacaaaggagccgtgaaacacgcaaggagcttctggcttctcagctttgagacctgggctctttggagcacagagaactttgatttttaagcttctacagatatggtcctagagactggggtcctcagacactcccgctgtatgtggtgagcttggctggaaacttgccatagccaggactgaggggctggccccaggacgctctcaagaaatggggctactgtggtttagacactcctgctgctcacattgatgggtggctattaaaggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 477-480 - skipped (4 entries)

SEQ ID NO: 481 - protein, 150 aa, synthetic construct; identical to SEQ ID NO: 373

SEQ ID NO: 482 - protein, 152 aa, synthetic construct; identical to SEQ ID NO: 371

SEQ ID NO: 483 - protein, 609 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGGSGGGGSGGGGSGGGGSGGGSLQVVISAILALVVLTVISLIILIGGGGSGKPIPNPLLGLDST

SEQ ID NO: 484 - protein, 495 aa, synthetic construct; identical to SEQ ID NO: 321

SEQ ID NO: 485 - protein, 604 aa, synthetic construct; identical to SEQ ID NO: 322

SEQ ID NO: 486 - protein, 756 aa, synthetic construct; identical to SEQ ID NO: 323

SEQ ID NO: 487 - protein, 948 aa, synthetic construct; identical to SEQ ID NO: 324

SEQ ID NO: 488 - protein, 445 aa, synthetic construct
MASPQLRGYGVQAIPVLLLLLLLLLLPLRVTPGTTCPPPVSIEHADIRVKNYSVNSRERYVCNSGFKRKAGTSTLIECVINKNTNVAHWTTPSLKCIRDPSLAHYSPVPTVVTPKVTSQPESPSPSAKEPEAFSPKSDTAMTTETAIMPGSRLTPSQTTSAGTTGTGSHKSSRAPSLAATMTLEPTASTSLRITEISPHSSKMTKVAISTSVLLVGAGVVMAFLAWYIKSRQPSQPCRVEVETMETVPMTVRASSKEDEDTGAGGSGGSGGSGGSGGSGGSGGMKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 489 - protein, 230 aa, synthetic construct
MEFGLSWVFLVALFRGVQCITCPPPMSVEHADIWVKSYSLYSRERYICNSGFKRKAGTSSLTECVLNKATNVAHWTTPSLKCIRDPALVHQRPAPPGGSGGSGGSGGSGGSGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 490 - protein, 184 aa, synthetic construct
MEFGLSWVFLVALFRGVQCCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 491-500 - skipped (10 entries)

SEQ ID NO: 501 - protein, 150 aa, synthetic construct; identical to SEQ ID NO: 373

SEQ ID NO: 502 - protein, 152 aa, synthetic construct; identical to SEQ ID NO: 371

SEQ ID NO: 503 - protein, 609 aa, synthetic construct; identical to SEQ ID NO: 483

SEQ ID NO: 504 - protein, 495 aa, synthetic construct; identical to SEQ ID NO: 321

SEQ ID NO: 505 - protein, 604 aa, synthetic construct; identical to SEQ ID NO: 322

SEQ ID NO: 506 - protein, 756 aa, synthetic construct; identical to SEQ ID NO: 323

SEQ ID NO: 507 - protein, 948 aa, synthetic construct; identical to SEQ ID NO: 324

SEQ ID NO: 508 - protein, 445 aa, synthetic construct; identical to SEQ ID NO: 488

SEQ ID NO: 509 - protein, 230 aa, synthetic construct; identical to SEQ ID NO: 489

SEQ ID NO: 510 - protein, 184 aa, synthetic construct; identical to SEQ ID NO: 490

SEQ ID NO: 511-550 - skipped (40 entries)

SEQ ID NO: 551 - RNA, 72 nt, synthetic construct
  source 1..72: mol_type = other RNA
aaacgcaagcaagaggaacaaatggaaactgagcattttgctctgtaaagcatttcagatgcagcttgtgtg

SEQ ID NO: 552 - RNA, 306 nt, synthetic construct
  source 1..306: mol_type = other RNA
gcgctgacccagctgatccagctctatcatcgcttccaccgggtgctgtcccagccgcagctccgagccctccctgcccgggctgagctcatcaacattcaccaccttatggtggagctcaagaagcataagcccaacttctgatgtgccagaaaccgccctgagatctgccggtcatctccatggacttctgcaccccattccatacccttcttcacctggggtaccccttccagttttccccttgcttcccaggcccttgacatggcttacctgccttcactcccagcaccttgcccaacagga

SEQ ID NO: 553 - RNA, 126 nt, synthetic construct
  source 1..126: mol_type = other RNA
cttccagcagaggagaacatgtctaacacgtgcctcaaaagcactggggagttagtagtgcagtggcatctgaaacctgtggagcagaaagcacatgagtcctaatgccccagcagcttccgattg

SEQ ID NO: 554 - RNA, 222 nt, synthetic construct
  source 1..222: mol_type = other RNA
atagtataccctggaattgctgtatttttccagaatgccttcatcttttttggagggctggtttttaagtttggccgcactgaagacttatggcagtgaacacatctgatttcccacagcacaacagccctgcatgggtttgtttgtttttttactgctcactcccaaccttttgtaatgccattttctaaacttatttctgagtgtagtctcagcttaaag

SEQ ID NO: 555 - RNA, 201 nt, synthetic construct
  source 1..201: mol_type = other RNA
ggtgacaaatccattcgtttccgtcccacgctggtcttcagggatcaccacgctcacctgttcctcaatattttcagtgacatcttagcagacttcaagtaaagaagccatttccactacagtgagaaagcccggatcccaacagttgtcaaattgattagtttgcctaattcatgttttcacttaaaagtatcagaggtg

SEQ ID NO: 556 - RNA, 264 nt, synthetic construct
  source 1..264: mol_type = other RNA
tgtcctttgggctctgtggagagctttagccttgcacggcggcgctgggaggcattgcctgccatgcccactgcccgctgctcctgctctagtctgcaggctgggccccggctgtttgttattgggggtgtggcccagggccccagtcaagccgtggaggcactgtgtctgcgtgatggggtctgaaggcttggtgggagctgtccactggagcagctcattgccagaggcagctatttctatggctccttttgctgctgagga

SEQ ID NO: 557 - RNA, 201 nt, synthetic construct
  source 1..201: mol_type = other RNA
atatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacct

SEQ ID NO: 558 - RNA, 201 nt, synthetic construct
  source 1..201: mol_type = other RNA
gaggttctccaggaccttaggtttgatgcggaatctgccgagtgatggcggctccccagggatgcgccgagggagatgggaaacggggcggatggcgcccagcccagccctaactgccagctggctggggttgcgccccactgcgctgctgaccttcctgcagttccagacacctcccacaataaagagctcctcctctgt

SEQ ID NO: 559 - RNA, 198 nt, synthetic construct
  source 1..198: mol_type = other RNA
ggtgaggtcaaggcaggagagaagagcctgagccagcacgtggaggccgtggacaagcggctggaacagagccagcccgagtacaaggcgctcttcaaagagatcttctccaggatccagaagaccaaggctgacatcaacgccaccaaagtcaagacgcacagcagcaagtgacccttctccggcctgcagcctccc

SEQ ID NO: 560 - RNA, 213 nt, synthetic construct
  source 1..213: mol_type = other RNA
gaggcgatcagcgaggttctccaggaccttaggtttgatgcggaatctgccgagtgatggcggctccccagggatgcgccgagggagatgggaaacggggcggatggcgcccagcccagccctaactgccagctggctggggttgcgccccactgcgctgctgaccttcctgcagttccagacacctcccacaataaagagctcctcctctgt

SEQ ID NO: 561-580 - skipped (20 entries)

SEQ ID NO: 581 - RNA, 201 nt, synthetic construct; identical to SEQ ID NO: 558
  source 1..201: mol_type = other RNA

SEQ ID NO: 582 - RNA, 201 nt, synthetic construct
  source 1..201: mol_type = other RNA
atcctagcttcggtgctggcagtgtcccaacaggaatacctagacagtatgaagaaaaacaaagtgcacagagacccgcccccagacaagagttgatggagacccagggattggacaccatctcccaaccccagtactcctgctctccggtgccacctcaccttctttggcttcttccctcttgcctccttctgttctttc

SEQ ID NO: 583 - RNA, 201 nt, synthetic construct
  source 1..201: mol_type = other RNA
actgtttcctatgatcctaggaaacccactgtgaagaaaattgccccaatgatggccaagacaattaaagctttcaagaacagattctcccgacgataaactgaggacttgccttggaaatggaatctggggaggcaggaatacaaggacagtgggggttggggaatggaattctacaggagactggagtcttgctttgtg

SEQ ID NO: 584 - RNA, 159 nt, synthetic construct
  source 1..159: mol_type = other RNA
ttgtcatgtgtacaggaaatcagtgatgtggtgcagaggtagccactgttagcctggtgggaaaatgcacacatttctgaggggagagggaaaaggacttgttttcctgtgttcttgttttcagaaaatgaaagactcatacttgagtgtgtttatgtg

SEQ ID NO: 585 - RNA, 228 nt, synthetic construct
  source 1..228: mol_type = other RNA
gtgcagcacaaaaagcccgccgacatccctcagggctccttggcctacctggagcaggcatctgccaacatccctgcacctctgaagccaacgtgagcaaagggcagaggcagttggcctatgagtgggctgatgcgtgaggttggccacacattccttcctgtggacttgacattttggaagaactctttgccagataatgagttcattttagttttatgctcccat

SEQ ID NO: 586 - RNA, 264 nt, synthetic construct
  source 1..264: mol_type = other RNA
tggggtccccagtggggaatgaacgggtacttcctcatcgagcgcggaaagaacatgtgtggcctggctgcctgcgcctcctaccccatccctctggtgtgagccgtggcagccgcagcgcagactggcggagaaggagaggaacgggcagcctgggcctgggtggaaatcctgccctggaggaagttgtggggagatccactgggacccccaacattctgccctcacctctgtgcccagcctggaaacctacagacaaggagg

SEQ ID NO: 587 - RNA, 249 nt, synthetic construct
  source 1..249: mol_type = other RNA
gaagaaacaaagaataaactcaaagccggcaatgccctggcatttttgggtcttgagagaaaacaatttgaatgactgaatttactacaaaggcaaactttcaaaaggatatctcttttttgtttccaaatatgtatcaacaggtatcaacaaaatcctattttgaactattttactcagaaaagaatatcccaaatatcccaaattattcataataaaaatgatttgaaagtgttttcattcttaaaa

SEQ ID NO: 588 - RNA, 195 nt, synthetic construct
  source 1..195: mol_type = other RNA
ttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaagatgtgcaaggagatttaaggatttcaaagaaaataaaggttctttgtttt

SEQ ID NO: 589 - RNA, 306 nt, synthetic construct
  source 1..306: mol_type = other RNA
aagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaa

SEQ ID NO: 590 - RNA, 230 nt, synthetic construct
  source 1..230: mol_type = other RNA
ccccgccctcccagtttccgcgcgcctctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgaga

SEQ ID NO: 591-600 - skipped (10 entries)

SEQ ID NO: 601 - protein, 511 aa, synthetic construct
MEFGLSWVFLVALFRGVQCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHPVGLLARVPLSLYSGHPVGLLARVPLSLYSGLSGRSDNHGGGSGGGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKHPVGLLARVPLSLYSGHPVGLLARVPLSLYSGLSGRSDNHGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 602 - protein, 348 aa, synthetic construct
MEFGLSWVFLVALFRGVQCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHPVGLLARVPLSLYSGHPVGLLARVPLSLYSGLSGRSDNHGGGSGGGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEK

SEQ ID NO: 603 - protein, 348 aa, synthetic construct
MEFGLSWVFLVALFRGVQCCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKHPVGLLARVPLSLYSGHPVGLLARVPLSLYSGLSGRSDNHGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 604 - protein, 639 aa, synthetic construct
MEFGLSWVFLVALFRGVQCENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKHGGGGSSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 605 - protein, 700 aa, synthetic construct
MEFGLSWVFLVALFRGVQCENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKHHPVGLLARVPLSLYSGHPVGLLARVPLSLYSGLSGRSDNHGGGSGGGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKHPVGLLARVPLSLYSGHPVGLLARVPLSLYSGLSGRSDNHGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 606 - protein, 185 aa, synthetic construct
MEFGLSWVFLVALFRGVQCCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEK

SEQ ID NO: 607 - protein, 445 aa, synthetic construct
MEFGLSWVFLVALFRGVQCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 608 - protein, 510 aa, synthetic construct
MEFGLSWVFLVALFRGVQCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHPVGLLARVPLSLYSGHPVGLLARVPLSLYSGLSGRSDNHGGGSGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEHPVGLLARVPLSLYSGHPVGLLARVPLSLYSGLSGRSDNHGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 609 - protein, 546 aa, synthetic construct
MEFGLSWVFLVALFRGVQCISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 610 - protein, 612 aa, synthetic construct
MEFGLSWVFLVALFRGVQCISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKHPVGLLARVPLSLYSGHPVGLLARVPLSLYSGLSGRSDNHGGGSGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEHPVGLLARVPLSLYSGHPVGLLARVPLSLYSGLSGRSDNHGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 611 - protein, 347 aa, synthetic construct
MEFGLSWVFLVALFRGVQCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHPVGLLARVPLSLYSGHPVGLLARVPLSLYSGLSGRSDNHGGGSGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 612 - protein, 347 aa, synthetic construct
MEFGLSWVFLVALFRGVQCCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEHPVGLLARVPLSLYSGHPVGLLARVPLSLYSGLSGRSDNHGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 613 - protein, 184 aa, synthetic construct; identical to SEQ ID NO: 490

SEQ ID NO: 614 - protein, 388 aa, synthetic construct; identical to SEQ ID NO: 351

SEQ ID NO: 615 - protein, 518 aa, synthetic construct; identical to SEQ ID NO: 352

SEQ ID NO: 616 - protein, 351 aa, synthetic construct; identical to SEQ ID NO: 353

SEQ ID NO: 617 - protein, 635 aa, synthetic construct; identical to SEQ ID NO: 358

SEQ ID NO: 618 - protein, 635 aa, synthetic construct; identical to SEQ ID NO: 358

SEQ ID NO: 619 - protein, 635 aa, synthetic construct; identical to SEQ ID NO: 361

SEQ ID NO: 620 - protein, 635 aa, synthetic construct; identical to SEQ ID NO: 361

SEQ ID NO: 621 - protein, 152 aa, synthetic construct; identical to SEQ ID NO: 371

SEQ ID NO: 622 - protein, 224 aa, synthetic construct
MSVLTQVLALLLLWLTGARCTTCPPPVSIEHADIRVKNYSVNSRERYVCNSGFKRKAGTSTLIECVINKNTNVAHWTTPSLKCIRDPSLAGGSGGSGGSGGSGGSGGSGGNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 623 - protein, 550 aa, synthetic construct
MSVLTQVLALLLLWLTGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHPVGLLARVPLSLYSGHPVGLLARVPLSLYSGLSGRSDNHGGGSGGGSTTCPPPVSIEHADIRVKNYSVNSRERYVCNSGFKRKAGTSTLIECVINKNTNVAHWTTPSLKCIRDPSLAGGSGGSGGSGGSGGSGGSGGNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSHPVGLLARVPLSLYSGHPVGLLARVPLSLYSGLSGRSDNHGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 624 - protein, 387 aa, synthetic construct
MSVLTQVLALLLLWLTGARCTTCPPPVSIEHADIRVKNYSVNSRERYVCNSGFKRKAGTSTLIECVINKNTNVAHWTTPSLKCIRDPSLAGGSGGSGGSGGSGGSGGSGGNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSHPVGLLARVPLSLYSGHPVGLLARVPLSLYSGLSGRSDNHGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 625 - protein, 387 aa, synthetic construct
MSVLTQVLALLLLWLTGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHPVGLLARVPLSLYSGHPVGLLARVPLSLYSGLSGRSDNHGGGSGGGSTTCPPPVSIEHADIRVKNYSVNSRERYVCNSGFKRKAGTSTLIECVINKNTNVAHWTTPSLKCIRDPSLAGGSGGSGGSGGSGGSGGSGGNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 626 - protein, 484 aa, synthetic construct
MSVLTQVLALLLLWLTGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSTTCPPPVSIEHADIRVKNYSVNSRERYVCNSGFKRKAGTSTLIECVINKNTNVAHWTTPSLKCIRDPSLAGGSGGSGGSGGSGGSGGSGGNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 627 - protein, 388 aa, synthetic construct; identical to SEQ ID NO: 351

SEQ ID NO: 628 - protein, 518 aa, synthetic construct; identical to SEQ ID NO: 352

SEQ ID NO: 629 - protein, 351 aa, synthetic construct; identical to SEQ ID NO: 353

SEQ ID NO: 630 - protein, 439 aa, synthetic construct; identical to SEQ ID NO: 354

SEQ ID NO: 631 - protein, 569 aa, synthetic construct; identical to SEQ ID NO: 355

SEQ ID NO: 632 - protein, 686 aa, synthetic construct; identical to SEQ ID NO: 356

SEQ ID NO: 633 - protein, 686 aa, synthetic construct; identical to SEQ ID NO: 357

SEQ ID NO: 634 - protein, 635 aa, synthetic construct; identical to SEQ ID NO: 358

SEQ ID NO: 635 - protein, 536 aa, synthetic construct; identical to SEQ ID NO: 359

SEQ ID NO: 636 - protein, 485 aa, synthetic construct; identical to SEQ ID NO: 360

SEQ ID NO: 637 - protein, 635 aa, synthetic construct; identical to SEQ ID NO: 361

SEQ ID NO: 638 - protein, 152 aa, synthetic construct; identical to SEQ ID NO: 371

SEQ ID NO: 639 - protein, 282 aa, synthetic construct; identical to SEQ ID NO: 372

SEQ ID NO: 640 - protein, 492 aa, synthetic construct; identical to SEQ ID NO: 311

SEQ ID NO: 641 - protein, 612 aa, synthetic construct; identical to SEQ ID NO: 312

SEQ ID NO: 642 - protein, 492 aa, synthetic construct; identical to SEQ ID NO: 311

SEQ ID NO: 643 - protein, 782 aa, synthetic construct; identical to SEQ ID NO: 313

SEQ ID NO: 644 - protein, 459 aa, synthetic construct; identical to SEQ ID NO: 341

SEQ ID NO: 645 - protein, 459 aa, synthetic construct; identical to SEQ ID NO: 341

SEQ ID NO: 646 - protein, 716 aa, synthetic construct; identical to SEQ ID NO: 343

SEQ ID NO: 647 - protein, 716 aa, synthetic construct; identical to SEQ ID NO: 343

SEQ ID NO: 648 - protein, 495 aa, synthetic construct; identical to SEQ ID NO: 321

SEQ ID NO: 649 - protein, 604 aa, synthetic construct; identical to SEQ ID NO: 322

SEQ ID NO: 650 - protein, 495 aa, synthetic construct; identical to SEQ ID NO: 321

SEQ ID NO: 651 - protein, 756 aa, synthetic construct; identical to SEQ ID NO: 323

SEQ ID NO: 652 - protein, 612 aa, synthetic construct; identical to SEQ ID NO: 312

SEQ ID NO: 653 - protein, 782 aa, synthetic construct; identical to SEQ ID NO: 313

SEQ ID NO: 654 - protein, 972 aa, synthetic construct; identical to SEQ ID NO: 314

SEQ ID NO: 655 - protein, 972 aa, synthetic construct; identical to SEQ ID NO: 314

SEQ ID NO: 656 - protein, 579 aa, synthetic construct; identical to SEQ ID NO: 342

SEQ ID NO: 657 - protein, 906 aa, synthetic construct; identical to SEQ ID NO: 344

SEQ ID NO: 658 - protein, 579 aa, synthetic construct; identical to SEQ ID NO: 342

SEQ ID NO: 659 - protein, 906 aa, synthetic construct; identical to SEQ ID NO: 344

SEQ ID NO: 660 - protein, 604 aa, synthetic construct; identical to SEQ ID NO: 322

SEQ ID NO: 661 - protein, 756 aa, synthetic construct; identical to SEQ ID NO: 323

SEQ ID NO: 662 - protein, 948 aa, synthetic construct; identical to SEQ ID NO: 324

SEQ ID NO: 663 - protein, 948 aa, synthetic construct; identical to SEQ ID NO: 324

SEQ ID NO: 664 - protein, 678 aa, synthetic construct
METDTLLLWVLLLWVPGSTGAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQHPVGLLARVPLSLYSGHPVGLLARVPLSLYSGLSGRSDNHGGGSGGGSAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRNNTGTWSPWSQPLTFRTRPADPMKEGGGGSGGGGSGGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 665 - protein, 653 aa, synthetic construct
METDTLLLWVLLLWVPGSTGAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQHPVGLLARVPLSLYSGHPVGLLARVPLSLYSGLSGRSDNHGGGSGGGSAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRGGGGSGGGGSGGGGSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 666 - protein, 169 aa, synthetic construct
METDTLLLWVLLLWVPGSTGAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 667 - protein, 661 aa, synthetic construct
MEFGLSWVFLVALFRGVQCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHPVGLLARVPLSLYSGHPVGLLARVPLSLYSGLSGRSDNHGGGSGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 668 - protein, 224 aa, synthetic construct
MSVLTQVLALLLLWLTGARCNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSGGSGGSGGSGGSGGSGGSGGTTCPPPVSIEHADIRVKNYSVNSRERYVCNSGFKRKAGTSTLIECVINKNTNVAHWTTPSLKCIRDPSLA

SEQ ID NO: 669 - protein, 468 aa, synthetic construct
MSVLTQVLALLLLWLTGARCNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSGGSGGSGGSGGSGGSGGSGGTTCPPPVSIEHADIRVKNYSVNSRERYVCNSGFKRKAGTSTLIECVINKNTNVAHWTTPSLKCIRDPSLAGGGGSGGGGSGGGGSGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 670-673 - skipped (4 entries)

SEQ ID NO: 674 - protein, 444 aa, synthetic construct
MEFGLSWVFLVALFRGVQCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 675-677 - skipped (3 entries)

SEQ ID NO: 678 - protein, 490 aa, synthetic construct
MEFGLSWVFLVALFRGVQCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSITCPPPMSVEHADIWVKSYSLYSRERYICNSGFKRKAGTSSLTECVLNKATNVAHWTTPSLKCIRDPALVHQRPAPPGGSGGSGGSGGSGGSGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 679-684 - skipped (6 entries)

SEQ ID NO: 685 - protein, 393 aa, synthetic construct
MEFGLSWVFLVALFRGVQCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHPVGLLARVPLSLYSGHPVGLLARVPLSLYSGLSGRSDNHGGGSGGGSITCPPPMSVEHADIWVKSYSLYSRERYICNSGFKRKAGTSSLTECVLNKATNVAHWTTPSLKCIRDPALVHQRPAPPGGSGGSGGSGGSGGSGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 686-700 - skipped (15 entries)

SEQ ID NO: 701 - RNA, 13 nt, Homo sapiens
  source 1..13: mol_type = mRNA
ctttaatgatgcg

SEQ ID NO: 702-703 - skipped (2 entries)

SEQ ID NO: 704 - RNA, 18 nt, Homo sapiens
  source 1..18: mol_type = mRNA
tgtgtgtgtgtgctcagg

SEQ ID NO: 705-720 - skipped (16 entries)

SEQ ID NO: 721 - RNA, 13 nt, synthetic construct
  source 1..13: mol_type = other RNA
ctgywrtradgyk

SEQ ID NO: 722-723 - skipped (2 entries)

SEQ ID NO: 724 - RNA, 18 nt, synthetic construct
  source 1..18: mol_type = other RNA
trtgygtntgtgctcagg

SEQ ID NO: 725-730 - skipped (6 entries)

SEQ ID NO: 731 - protein, 5 aa, synthetic construct
GGGGS

SEQ ID NO: 732 - protein, 5 aa, synthetic construct
GGSGG

SEQ ID NO: 733 - protein, 19 aa, synthetic construct
MEFGLSWVFLVALFRGVQC

SEQ ID NO: 734 - RNA, 63 nt, synthetic construct
  source 1..63: mol_type = other RNA
cctcagacactcccgctgtatgtggtgagcttggctggaaacttgccatagccaggactgagg

(19) United States
(12) Patent Application Publication
(10) Pub. No.: US 2026/0132180 A1
(43) Pub. Date: May 14, 2026

(54) ONCOSELECTIVE THERAPY

(71) Applicant: Kernal Biologics, Inc., Boston, MA (US)

(72) Inventors: Burak YILMAZ, Cambridge, MA (US); Cafer OZDEMIR, Cambridge, MA (US); Yusuf ERKUL, Cambridge, MA (US); Thomas ADDISON, Cambridge, MA (US); Isaak MUELLER, Cambridge, MA (US); Kelly BROCK, Cambridge, MA (US); Sadik YILDIZ, Belmont, MA (US); William Max ALEXANDER, Arlington, MA (US); Manfred KRAUS, San Diego, CA (US); Yulia RYBAKOVA, Cambridge, MA (US)

(21) Appl. No.: 19/382,788

(22) Filed: Nov. 7, 2025

Related U.S. Application Data
(63) Continuation of application No. PCT/US2024/028670, filed on May 9, 2024.
(60) Provisional application No. 63/501,377, filed on May 10, 2023.
(60) Provisional application No. 63/501,259, filed on May 10, 2023.
(60) Provisional application No. 63/535,802, filed on Aug. 31, 2023.

(51) Int. Cl. C07K 14/54; A61K 9/1272; A61K 9/51; A61K 31/7105; A61K 38/00; A61P 35/00; C07K 14/55; C07K 14/56

(52) U.S. Cl. CPC C07K 14/5443; A61K 9/1272; A61K 9/5123; A61K 31/7105; A61P 35/00; C07K 14/5434; C07K 14/55; C07K 14/56; A61K 38/00

(57) ABSTRACT

The present disclosure provides compositions and methods for treatment of diseases or conditions.

### CROSS-REFERENCE

This application is a continuation of International Application No. PCT/US2024/028670, filed May 9, 2024, which claims the benefit of U.S. Provisional Application Ser. No. 63/501,259, filed on May 10, 2023, U.S. Provisional Application Ser. No. 63/501,377, filed on May 10, 2023, and U.S. Provisional Application Ser. No. 63/535,802, filed on Aug. 31, 2023, the entirety of which is hereby incorporated by reference herein.

### BACKGROUND

There is a need to develop improved therapies for the treatment of cancer. Therapies tailored to specifically target cancer cells can provide an opportunity for unique treatment options.

### SEQUENCE LISTING

The instant application contains a Sequence Listing which has been submitted electronically and is hereby incorporated by reference in its entirety. Said copy, created on Nov. 5, 2025, is named 61600-707.301_Sequence_Listing.xml and is 522,202 bytes in size.

### SUMMARY

The present disclosure provides compositions and methods for the treatment of a disease or condition such as cancer. Described herein, in some aspects, is an engineered polynucleotide comprising: at least one oncoselective sequence; and a coding sequence encoding an engineered therapeutic, wherein the at least one oncoselective sequence comprises a nucleic acid sequence that is: at least 75% identical to any one of SEQ ID NOs: 1-97 or 101-146. In some embodiments, the at least one oncoselective sequence comprises the nucleic acid sequence that is at least 80%, at least 85%, at least 90%, at least 95%, or at least 99% identical to any one of SEQ ID NOs: 1-97 or 101-146. In some embodiments, the at least one oncoselective sequence comprises the nucleic acid sequence that is any one of SEQ ID NOs: 1-97 or 101-146. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-75, 91-97, or 101-146. In some embodiments, the at least one oncoselective sequence comprises the nucleic acid sequence that is at least 5 contiguous nucleotides identical to any one of SEQ ID NOs: 76-90. In some embodiments, the at least one oncoselective sequence comprises an untranslated region (UTR). In some embodiments, the at least one oncoselective sequence comprises a nucleic acid motif. In some embodiments, the engineered further comprises two oncoselective sequences. In some embodiments, the two oncoselective sequences flank the coding sequence. In some embodiments, the two oncoselective sequences each comprises the nucleic acid sequence that is: at least 75% identical to any one of SEQ ID NOs: 1-75, 91-97, or 101-146; or any one of SEQ ID NOs: 76-90. In some embodiments, the at least one oncoselective sequence comprises at least one miRNA binding site or at least one protein binding site. In some embodiments, the at least one protein binding site is a RNA binding protein (RBP) site. In some embodiments, the engineered polynucleotide further comprises at least one nucleic acid modification. In some embodiments, the at least one nucleic acid modification comprises a methylation. In some embodiments, the at least one nucleic acid modification comprises a pseudouridine. In some embodiments, the at least one nucleic acid modification comprises an N1-Methylpseudouridine. In some embodiments, the engineered therapeutic comprises a cytokine. In some embodiments, the engineered therapeutic is encoded from a nucleic acid sequence that is at least 75%, at least 80%, at least 85%, at least 90%, at least 95%, or at least 99% identical to any one of SEQ ID NOs: 461-476. In some embodiments, the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 351-361, 371-373, 481-490, or 501-510. In some embodiments, the cytokine comprises an engineered cytokine comprising a secreted cytokine, a membrane tethered cytokine, a masked cytokine, a cytokine fusion, or a combination thereof. In some embodiments, the cytokine comprises an interleukin or an interferon. In some embodiments, the cytokine comprises the interleukin. In some embodiments, the interleukin comprises an IL-2. In some embodiments, the IL-2 is encoded from a nucleic acid sequence that is at least 75%, at least 80%, at least 85%, at least 90%, at least 95%, or at least 99% identical to SEQ ID NOs: 467 or 468. In some embodiments, the IL-2 comprises an amino acid sequence that is at least 75%, at least 80%, at least 85%, at least 90%, at least 95%, or at least 99% identical to any one of SEQ ID NOs: 351-361, 371-373, 481, 482, 501, 502, 614-621, or 664-667. In some embodiments, the interleukin comprises an IL-12. In some embodiments, the IL-12 is encoded from a nucleic acid sequence that is at least 75%, at least 80%, at least 85%, at least 90%, at least 95%, or at least 99% identical to any one of SEQ ID NOs: 461, 462, 475, or 476. In some embodiments, the IL-12 comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, 503-507, or 648-663. In some embodiments, the interleukin comprises an IL-15. In some embodiments, the IL-15 is encoded from a nucleic acid sequence that is at least 75%, at least 80%, at least 85%, at least 90%, at least 95%, or at least 99% identical to any one of SEQ ID NOs: 463, 464, 469, or 470. In some embodiments, the IL-15 comprises an amino acid sequence that is at least 75%, at least 80%, at least 85%, at least 90%, at least 95%, or at least 99% identical to SEQ ID NOs: 488, 489, 508, 509, 622-647, 668, or 669. In some embodiments, the cytokine comprises the interferon. In some embodiments, the interferon comprises an IFN-α. In some embodiments, the IFN-α is encoded from a nucleic acid sequence that is at least 75%, at least 80%, at least 85%, at least 90%, at least 95%, or at least 99% identical to SEQ ID NOs: 465 or 466. In some embodiments, the IFN-α comprises an amino acid sequence that is at least 75%, at least 80%, at least 85%, at least 90%, at least 95%, or at least 99% identical to SEQ ID NOs: 490, 510, 601-613, 674, 678, or 685. In some embodiments, the engineered therapeutic comprises a masking domain. In some embodiments, the engineered therapeutic comprises a half-life extension domain. In some embodiments, the half-life extension domain comprises antibody or fragment thereof a serum binding protein or fragment thereof. In some embodiments, the engineered therapeutic comprises a linker. In some embodiments, the linker is a cleavable linker. In some embodiments, the engineered therapeutic comprises a signal peptide. In some embodiments, the engineered therapeutic comprises an engineered cytokine comprising modified cytokine comprising a secreted cytokine, a membrane tethered cytokine, a masked cytokine, a cytokine fusion, or a combination thereof. In some embodiments, the cytokine fusion comprises a cytokine operatively coupled to a half-life extension domain. In some embodiments, the half-life extension domain comprises an antibody or fragment thereof or a serum binding protein or fragment thereof. In some embodiments, the at least one oncoselective sequence comprises a secondary structure. In some embodiments, the secondary structure comprises a stem loop; a bulge loop, a pseudoknot, or a combination thereof. In some embodiments, the stem loop comprises a double stem loop. In some embodiments, the at least one oncoselective sequence comprises a truncation. In some embodiments, the engineered polynucleotide comprises RNA. In some embodiments, the engineered polynucleotide decreases immunogenicity. In some embodiments, the at least one oncoselective sequence increases an expression of the engineered therapeutic in a cancer cell compared to a comparable expression of the engineered therapeutic in a non-cancer cell. In some embodiments, the at least one oncoselective sequence increases the expression of the engineered therapeutic in a cancer cell compared to the comparable expression of the engineered therapeutic in a non-cancer cell by at least 10%, at least 20%, at least 30%, at least 40%, at least 50%, at least 100%, at least 200%, at least 500%, or more. In some embodiments, the at least one oncoselective sequence increases a killing efficiency of a cancer cell compared to a comparable killing efficiency of a non-cancer cell.

Described herein, in some aspects, is a composition comprising the engineered polynucleotide described herein. In some embodiments, the composition comprises two or more of the engineered polynucleotides described herein. In some embodiments, the two or more of the engineered polynucleotides encode two or more cytokines comprising IL-2, IL-12, IL-15, IFN-α, or a combination thereof. In some embodiments, the two or more of the engineered polynucleotides encode two or more cytokines comprising IL-2, IL-12, IL-15, and IFN-α. In some embodiments, the composition further comprises at least one additional active ingredient. In some embodiments, the at least one additional active ingredient comprises an immune checkpoint inhibitor. In some embodiments, the immune checkpoint inhibitor comprises one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, CD137, or a combination thereof. In some embodiments, the immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab. In some embodiments, the composition comprises contacting the engineered polynucleotide with a lipid. In some embodiments, the lipid comprises a lipid nanoparticle (LNP).

Described herein, in some aspects, is an RNA encoding the engineered polynucleotide described herein.

Described herein, in some aspects, is a vector encoding the engineered polynucleotide described herein.

Described herein, in some aspects, is a cell comprising the engineered polynucleotide described herein. In some embodiments, the cell comprises an autologous cell or an allogenic cell.

Described herein, in some aspects, is a pharmaceutical composition comprising: the engineered polynucleotide described herein, the composition described herein, the RNA described herein, the vector described herein, or the cell described herein; and at least one carrier, excipient, or diluent. In some embodiments, the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, or a combination thereof. In some embodiments, the pharmaceutical composition comprises at least one additional active ingredient.

Described herein, in some aspects, is a method of administering an engineered polynucleotide a subject, comprising administering the engineered polynucleotide described herein, the composition described herein, the RNA described herein, the vector described herein, or the cell described herein, or the pharmaceutical composition described herein to the subject.

Described herein, in some aspects, is a method for treating a disease or condition in a subject comprising administering the engineered polynucleotide described herein, the composition described herein, the RNA described herein, the vector described herein, or the cell described herein, or the pharmaceutical composition described herein to the subject, thereby treating the disease or condition. In some embodiments, the disease or condition comprises cancer.

Described herein, in some aspects, is a method for treating a disease or condition in a subject, comprising administering to the subject an engineered polynucleotide, said engineered polynucleotide comprises at least one oncoselective sequence; and a coding sequence encoding an engineered therapeutic, wherein the at least one oncoselective sequence comprises a nucleic acid sequence that is: at least 75% identical to any one of SEQ ID NOs: 1-97 or 101-146. In some embodiments, the at least one oncoselective sequence comprises the nucleic acid sequence that is at least 80%, at least 85%, at least 90%, at least 95%, or at least 99% identical to any one of SEQ ID NOs: 1-97 or 101-146. In some embodiments, the at least one oncoselective sequence comprises the nucleic acid sequence that is any one of SEQ ID NOs: 1-97 or 101-146. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-75, 91-97, or 101-146. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-8, 91-97, or 101-146.

In some embodiments, the at least one oncoselective sequence comprises the nucleic acid sequence that is at least 5 contiguous nucleotides identical to any one of SEQ ID NOs: 76-90. In some embodiments, the engineered therapeutic comprises a cytokine. In some embodiments, the cytokine comprises an engineered cytokine comprising a secreted cytokine, a membrane tethered cytokine, a masked cytokine, a cytokine fusion, or a combination thereof. In some embodiments, the cytokine comprises an interleukin or an interferon. In some embodiments, the cytokine comprises the interleukin. In some embodiments, the interleukin comprises an IL-2. In some embodiments, the IL-2 is encoded from a nucleic acid sequence that is at least 75%, at least 80%, at least 85%, at least 90%, at least 95%, or at least 99% identical to any one of SEQ ID NOs: 467 or 468. In some embodiments, the IL-2 comprises an amino acid sequence that is at least 75%, at least 80%, at least 85%, at least 90%, at least 95%, or at least 99% identical to any one of SEQ ID NOs: 351-361, 371-373, 481, 482, 501, 502, 614-621, or 664-667. In some embodiments, the interleukin comprises an IL-12. In some embodiments, the IL-12 is encoded from a nucleic acid sequence that is at least 75%, at least 80%, at least 85%, at least 90%, at least 95%, or at least 99% identical to any one of SEQ ID NOs: 461, 462, 475, or 476. In some embodiments, the IL-12 comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, 503-507, or 648-663. In some embodiments, the interleukin comprises an IL-15. In some embodiments, the IL-15 is encoded from a nucleic acid sequence that is at least 75%, at least 80%, at least 85%, at least 90%, at least 95%, or at least 99% identical to any one of SEQ ID NOs: 463, 464, 469, or 470. In some embodiments, the IL-15 comprises an amino acid sequence that is at least 75%, at least 80%, at least 85%, at least 90%, at least 95%, or at least 99% identical to SEQ ID NOs: 488 489, 508, 509, 622-647, 668, or 669. In some embodiments, the cytokine comprises the interferon. In some embodiments, the interferon comprises an IFN-α. In some embodiments, the IFN-α is encoded from a nucleic acid sequence that is at least 75%, at least 80%, at least 85%, at least 90%, at least 95%, or at least 99% identical to any one of SEQ ID NOs: 465 or 466. In some embodiments, the IFN-α comprises an amino acid sequence that is at least 75%, at least 80%, at least 85%, at least 90%, at least 95%, or at least 99% identical to SEQ ID NOs: 490, 510, 601-613, 674, 678, or 685. In some embodiments, the disease or condition comprises solid tumor. In some embodiments, the disease or condition comprises cancer. In some embodiments, the cancer comprises melanoma, breast cancer, basal cell carcinoma (BCC), squamous cell carcinoma (SCC), cutaneous SCC (cSCC or CSCC), Head & Neck Cancer, Thyroid Cancer, Colorectal Cancer, Prostate Cancer, Liver cancer, Pancreatic Cancer, Renal Cell Carcinoma, Brain Cancer, Soft Tissue Sarcoma, Lung Cancer, or a combination thereof. In some embodiments, the cancer does not respond to immune checkpoint inhibitor treatment. In some embodiments, the at least one oncoselective sequence increases expression of the engineered therapeutic in a cancer cell compared to expression of the engineered therapeutic in a non-cancer cell.

Described herein, in some aspects, is an engineered polynucleotide comprising: at least one oncoselective sequence; and a coding sequence encoding an engineered therapeutic, wherein the at least one oncoselective sequence comprises a nucleic acid sequence that is: at least 75% identical to any one of SEQ ID NOs: 1-97 or SEQ ID NOs: 101-146. In some embodiments, the at least one oncoselective sequence comprises the nucleic acid sequence that is at least 75% identical to any one of SEQ ID NOs: 1-8. In some embodiments, the at least one oncoselective sequence comprises the nucleic acid sequence that is at least 80%, at least 85%, at least 90%, at least 95%, or at least 99% identical to any one of SEQ ID NOs: 1-8. In some embodiments, the at least one oncoselective sequence comprises the nucleic acid sequence that is any one of SEQ ID NOs: 1-8. In some embodiments, the at least one oncoselective sequence comprises the nucleic acid sequence that is at least 50 contiguous nucleotides, at least 100 contiguous nucleotides, or at least 150 contiguous nucleotides identical to any one SEQ ID NOs: 1-97 or SEQ ID NOs: 101-146. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-8. In some embodiments, the at least one oncoselective sequence comprises the nucleic acid sequence that is at least 5 contiguous nucleotides identical to any one of SEQ ID NOs: 1-90 or SEQ ID NOs: 101-146. In some embodiments, the at least one oncoselective sequence comprises an untranslated region (UTR). In some embodiments, the at least one oncoselective sequence comprises a nucleic acid motif. In some embodiments, the engineered polynucleotide further comprises two oncoselective sequences. In some embodiments, the two oncoselective sequences flank the coding sequence. In some embodiments, the at least one oncoselective sequence comprises at least one miRNA binding site. In some embodiments, the at least one oncoselective sequence comprises at least one protein binding site. In some embodiments, the at least one protein binding site is a RNA binding protein (RBP) site. In some embodiments, the engineered polynucleotide comprises at least one nucleic acid modification. In some embodiments, the at least one nucleic acid modification comprises a methylation. In some embodiments, the at least one nucleic acid modification comprises a pseudouridine. In some embodiments, the at least one nucleic acid modification comprises an N1-Methylpseudouridine. In some embodiments, the engineered therapeutic comprises a cytokine. In some embodiments, the cytokine comprises an engineered cytokine comprising a secreted cytokine, a membrane tethered cytokine, a masked cytokine, a cytokine fusion, or a combination thereof. In some embodiments, the cytokine comprises an interleukin or an interferon. In some embodiments, the cytokine comprises the interleukin. In some embodiments, the interleukin comprises an IL-2. In some embodiments, the interleukin comprises an IL-12. In some embodiments, the interleukin comprises an IL-15. In some embodiments, the cytokine comprises the interferon. In some embodiments, the interferon comprises an IFN-α. In some embodiments, the at least one oncoselective sequence comprises a secondary structure. In some embodiments, the secondary structure comprises a stem loop; a bulge loop, a pseudoknot, or a combination thereof. In some embodiments, the stem loop comprises a double stem loop. In some embodiments, the at least one oncoselective sequence comprises a truncation. In some embodiments, the engineered polynucleotide comprises RNA. In some embodiments, the engineered polynucleotide decreases immunogenicity. In some embodiments, the at least one oncoselective sequence increases expression of the engineered therapeutic in a cancer cell compared to expression of the engineered therapeutic in a normal cell.

Described herein, in some aspects, is a composition comprising an engineered polynucleotide described herein. In some embodiments, the composition comprises two or more of the engineered polynucleotides described herein. In some embodiments, the two or more of the engineered polynucleotides encode two or more cytokines comprising IL-2, IL-12, IL-15, IFN-α, or a combination thereof. In some embodiments, the two or more of the engineered polynucleotides encode two or more cytokines comprising IL-2, IL-12, IL-15, and IFN-α. In some embodiments, the composition further comprises at least one additional active ingredient. In some embodiments, the at least one additional active ingredient comprises an immune checkpoint inhibitor. In some embodiments, the immune checkpoint inhibitor comprises one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, CD137, or a combination thereof. In some embodiments, the immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab. In some embodiments, the composition comprises contacting the engineered polynucleotide with a lipid. In some embodiments, the lipid comprises a lipid nanoparticle (LNP).

Described herein, in some aspects, is a vector encoding an engineered polynucleotide described herein.

Described herein, in some aspects, is a cell comprising a vector described herein or an engineered polynucleotide described herein. In some embodiments, the cell comprises an autologous cell or an allogenic cell.

Described herein, in some aspects, is a pharmaceutical composition comprising: an engineered polynucleotide described herein, a composition described herein, a vector described herein, or a cell described herein; and at least one carrier, excipient, or diluent. In some embodiments, the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, or a combination thereof. In some embodiments, the pharmaceutical composition comprises at least one additional active ingredient.

Described herein, in some aspects, is a method of administering an engineered polynucleotide a subject, comprising administering an engineered polynucleotide described herein, a composition described herein, a vector described herein, or a cell described herein, or a pharmaceutical composition described herein to the subject.

Described herein, in some aspects, is a method for treating a disease or condition in a subject comprising administering an engineered polynucleotide described herein, a composition described herein, a vector described herein, or a cell described herein, or a pharmaceutical composition described herein to the subject, thereby treating the disease of condition.

Described herein, in some aspects, is a method for treating a disease or condition in a subject, comprising administering to the subject an engineered polynucleotide, said engineered polynucleotide comprises at least one oncoselective sequence; and a coding sequence encoding an engineered therapeutic, wherein the at least one oncoselective sequence comprises a nucleic acid sequence that is: at least 75% identical to any one SEQ ID NOs: 1-97 or SEQ ID NOs: 101-146. In some embodiments, the at least one oncoselective sequence comprises the nucleic acid sequence that is at least 75% identical to any one of SEQ ID NOs: 1-8. In some embodiments, the at least one oncoselective sequence comprises the nucleic acid sequence that is at least 80%, at least 85%, at least 90%, at least 95%, or at least 99% identical to any one of SEQ ID NOs: 1-8. In some embodiments, the at least one oncoselective sequence comprises the nucleic acid sequence that is any one of SEQ ID NOs: 1-8. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-97 or SEQ ID NOs: 101-146. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-8. In some embodiments, the at least one oncoselective sequence comprises the nucleic acid sequence that is at least 5 contiguous nucleotides identical to any one of SEQ ID NOs: 1-97 or SEQ ID NOs: 101-146. In some embodiments, the engineered therapeutic comprises a cytokine. In some embodiments, the cytokine comprises an engineered cytokine comprising a secreted cytokine, a membrane tethered cytokine, a masked cytokine, a cytokine fusion, or a combination thereof. In some embodiments, the cytokine comprises an interleukin or an interferon. In some embodiments, the cytokine comprises the interleukin. In some embodiments, the interleukin comprises an IL-2. In some embodiments, the interleukin comprises an IL-12. In some embodiments, the interleukin comprises an IL-15. In some embodiments, the interferon comprises an IFN-α. In some embodiments, the disease or condition comprises solid tumor. In some embodiments, the disease or condition comprises cancer. In some embodiments, the cancer comprises melanoma, breast cancer, basal cell carcinoma (BCC), squamous cell carcinoma (SCC), cutaneous SCC (cSCC or CSCC), Head & Neck Cancer, Thyroid Cancer, Colorectal Cancer, Prostate Cancer, Liver cancer, Pancreatic Cancer, Renal Cell Carcinoma, Brain Cancer, Soft Tissue Sarcoma, Lung Cancer, or a combination thereof. In some embodiments, the cancer does not respond to immune checkpoint inhibitor treatment. In some embodiments, the at least one oncoselective sequence increases expression of the engineered therapeutic in a cancer cell compared to expression of the engineered therapeutic in a normal cell.

Described herein, in some aspects, is an engineered polynucleotide comprising: at least one oncoselective sequence; and a coding sequence encoding an engineered therapeutic. In some embodiments, the engineered therapeutic comprises: an engineered interleukin or fragment thereof; or an engineered subunit of an interleukin. In some embodiments, the engineered interleukin or fragment thereof comprises an engineered IL-12 or fragment thereof or an engineered subunit of IL-12. In some embodiments, the engineered interleukin or fragment thereof comprises an engineered IL-2 or fragment thereof. In some embodiments, the engineered therapeutic comprises a masking domain. In some embodiments, the engineered therapeutic comprises a half-life extension domain. In some embodiments, the half-life extension domain comprises antibody or fragment thereof a serum binding protein or fragment thereof. In some embodiments, the engineered therapeutic comprises a linker. In some embodiments, the linker is a cleavable linker. In some embodiments, the engineered therapeutic comprises a signal peptide. In some embodiments, the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, or 648-663. In some embodiments, the engineered therapeutic comprises an amino acid sequence that is any one of SEQ ID NOs: 301-303, 311-314, 321-328, 331-335, 341-344, 461-476, 483-487, or 648-663. In some embodiments, the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one SEQ ID NOs: 301-303, 311-314, 321-328, 331-335, 341-344, 483-487, or 648-663. In some embodiments, the engineered therapeutic comprises an amino acid sequence that is at least 75%, at least 80%, at least 85%, at least 90%, at least 95%, or at least 99% identical to any one of SEQ ID NOs: 301-303. In some embodiments, the engineered therapeutic comprises an amino acid sequence that is at any one of SEQ ID NOs: 301-303. In some embodiments, the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 301-303. In some embodiments, the engineered therapeutic comprises an amino acid sequence that is at least 75%, at least 80%, at least 85%, at least 90%, at least 95%, or at least 99% identical to any one of SEQ ID NOs: 311-314 or 321-328. In some embodiments, the engineered therapeutic comprises an amino acid sequence that is at any one of SEQ ID NOs: 311-314 or 321-328. In some embodiments, the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 311-314 or 321-328. In some embodiments, the engineered therapeutic comprises an amino acid sequence that is at least 75%, at least 80%, at least 85%, at least 90%, at least 95%, or at least 99% identical to any one of SEQ ID NOs: 351-361 or 363. In some embodiments, the engineered therapeutic comprises an amino acid sequence that is at any one of SEQ ID NOs: 351-361 or 363. In some embodiments, the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 351-361 or 363. In some embodiments, the at least one oncoselective sequence comprises a nucleic acid motif. In some embodiments, the nucleic acid motif comprises an oncoselective readthrough motif. In some embodiments, the at least one oncoselective sequence comprises an untranslated region (UTR). In some embodiments, the UTR is a 5′ UTR or a 3′ UTR. In some embodiments, the at least one oncoselective sequence comprises a nucleic acid sequence that is at least 75%, at least 80%, at least 85%, at least 90%, at least 95%, or at least 99% identical to any one of SEQ ID NOs: 1-75, 91-97, or 101-146; or any one of SEQ ID NOs: 76-90. In some embodiments, the at least one oncoselective sequence comprises a nucleic acid sequence that is at least 50 contiguous nucleotides, at least 100 contiguous nucleotides, or at least 150 contiguous nucleotides identical to any one of SEQ ID NOs: 1-75, 91-97, or 101-146; or at least 5 contiguous nucleotides identical to any one of SEQ ID NOs: 76-90. In some embodiments, the engineered polynucleotide comprises two oncoselective sequences. In some embodiments, the two oncoselective sequences flank the coding sequence. In some embodiments, the at least one oncoselective sequence comprises at least one miRNA binding site. In some embodiments, the at least one oncoselective sequence comprises at least one protein binding site. In some embodiments, the at least one protein binding site is a RNA binding protein (RBP) site. In some embodiments, the engineered polynucleotide further comprises at least one nucleic acid modification. In some embodiments, the at least one nucleic acid modification comprises a methylation. In some embodiments, the at least one nucleic acid modification comprises a pseudouridine. In some embodiments, the at least one nucleic acid modification comprises an N1-Methylpseudouridine. In some embodiments, the engineered therapeutic comprises an engineered cytokine comprising modified cytokine comprising a secreted cytokine, a membrane tethered cytokine, a masked cytokine, a cytokine fusion, or a combination thereof. In some embodiments, the cytokine fusion comprises a cytokine coupled to a half-life extension domain. In some embodiments, the half-life extension domain comprises an antibody or fragment thereof or a serum binding protein or fragment thereof. In some embodiments, the engineered cytokine comprises an interleukin or an interferon. In some embodiments, the engineered cytokine comprises the interleukin. In some embodiments, the interleukin comprises an IL-2. In some embodiments, the interleukin comprises an IL-12. In some embodiments, the interleukin comprises an IL-15. In some embodiments, the interferon comprises an IFN-α. In some embodiments, the at least one oncoselective sequence comprises a secondary structure. In some embodiments, the secondary structure comprises a stem loop; a bulge loop, a pseudoknot, or a combination thereof. In some embodiments, the stem loop comprises a double stem loop. In some embodiments, the at least one oncoselective sequence comprises a truncation. In some embodiments, the engineered polynucleotide comprises RNA. In some embodiments, the engineered polynucleotide comprises reduced immunogenicity. In some embodiments, the at least one oncoselective sequence increases expression of the engineered therapeutic in a cancer cell compared to expression of the engineered therapeutic in a normal cell.

Described herein, in some aspects, is a composition comprising an engineered polynucleotide described herein. In some embodiments, the composition further comprises at least one additional polynucleotide encoding a cytokine. In some embodiments, the cytokine comprises IL-2, IL-12, IL-15, IFN-α, or a combination thereof.

Described herein, in some aspects, is a composition comprising two or more of the engineered polynucleotides described herein. In some embodiments, the two or more of the engineered polynucleotide encode two or more of the engineered therapeutic. In some embodiments, the two or more of the engineered therapeutic comprise: an amino acid sequence that is at least 75%, at least 80%, at least 85%, at least 90%, at least 95%, or at least 99% identical to any one of SEQ ID NOs: 301-303; and an amino acid sequence that is at least 75%, at least 80%, at least 85%, at least 90%, at least 95%, or at least 99% identical to any one of SEQ ID NOs: 351-361 or 363. In some embodiments, the two or more of the engineered therapeutic comprise: an amino acid sequence that is at least 75%, at least 80%, at least 85%, at least 90%, at least 95%, or at least 99% identical to any one of SEQ ID NOs: 311, 313, 321, 323, 325, or 327; an amino acid sequence that is at least 75%, at least 80%, at least 85%, at least 90%, at least 95%, or at least 99% identical to any one of SEQ ID NOs: 312, 314, 322, 324, 326, or 328; and an amino acid sequence that is at least 75%, at least 80%, at least 85%, at least 90%, at least 95%, or at least 99% identical to any one of SEQ ID NOs: 351-361 or 363. In some embodiments, the composition further comprises at least one additional polynucleotide encoding a cytokine. In some embodiments, the cytokine comprises IL-2, IL-12, IL-15, IFN-α, or a combination thereof. In some embodiments, the composition further comprises at least one additional active ingredient. In some embodiments, the at least one additional active ingredient comprises an immune checkpoint inhibitor. In some embodiments, the immune checkpoint inhibitor comprises one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, CD137, or a combination thereof. In some embodiments, the immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab. In some embodiments, the composition comprises contacting the engineered polynucleotide with a lipid. In some embodiments, the lipid comprises a lipid nanoparticle (LNP).

Described herein, in some aspects, is a vector encoding an engineered polynucleotide described herein.

Described herein, in some aspects, is a cell comprising a vector described herein or an engineered polynucleotide described herein. In some embodiments, the cell comprises an autologous cell or an allogenic cell.

Described herein, in some aspects, is a pharmaceutical composition comprising: an engineered polynucleotide described herein, a composition described herein, a vector described herein, or a cell described herein; and at least one carrier, excipient, or diluent. In some embodiments, the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, or a combination thereof. In some embodiments, the pharmaceutical composition further comprises at least one additional active ingredient.

Described herein, in some aspects, is a method of administering an engineered polynucleotide a subject, comprising administering an engineered polynucleotide described herein, a composition described herein, a vector described herein, a cell described herein, or a pharmaceutical composition described herein to the subject.

Described herein, in some aspects, is a method for treating a disease or condition in a subject, comprising administering an engineered polynucleotide described herein, a composition described herein, a vector described herein, a cell described herein, or a pharmaceutical composition described herein to the subject, thereby treating the disease of condition.

Described herein, in some aspects, is a method for treating a disease or condition in a subject, comprising administering to the subject an engineered polynucleotide, said engineered polynucleotide comprises: at least one oncoselective sequence; and a coding sequence encoding an engineered therapeutic. In some embodiments, the engineered therapeutic comprises: an engineered interleukin or fragment thereof; or an engineered subunit of an interleukin. In some embodiments, the engineered interleukin or fragment thereof comprises an engineered IL-12 or fragment thereof or an engineered subunit of IL-12. In some embodiments, the engineered interleukin or fragment thereof comprises an engineered IL-2 or fragment thereof. In some embodiments, the engineered therapeutic comprises a masking domain. In some embodiments, the engineered therapeutic comprises a half-life extension domain. In some embodiments, the half-life extension domain comprises an antibody or fragment thereof a serum binding protein or fragment thereof. In some embodiments, the engineered therapeutic comprises a linker. In some embodiments, the linker is a cleavable linker. In some embodiments, the engineered therapeutic comprises a signal peptide. In some embodiments, the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, or 648-663. In some embodiments, the engineered therapeutic comprises an amino acid sequence that is any one of SEQ ID NOs: 301-303, 311-314, 321-328, 331-335, 341-344, 483-487, or 648-663. In some embodiments, the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 301-303, 311-314, 321-328, 331-335, 341-344, 483-487, or 648-663. In some embodiments, the engineered therapeutic comprises an amino acid sequence that is at least 75%, at least 80%, at least 85%, at least 90%, at least 95%, or at least 99% identical to any one of SEQ ID NOs: 301-303. In some embodiments, the engineered therapeutic comprises an amino acid sequence that is at any one of SEQ ID NOs: 301-303. In some embodiments, the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 301-303. In some embodiments, the engineered therapeutic comprises an amino acid sequence that is at least 75%, at least 80%, at least 85%, at least 90%, at least 95%, or at least 99% identical to any one of SEQ ID NOs: 311-314 or 321-328. In some embodiments, the engineered therapeutic comprises an amino acid sequence that is at any one of SEQ ID NOs: 311-314 or 321-328. In some embodiments, the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 311-314 or 321-328. In some embodiments, the engineered therapeutic comprises an amino acid sequence that is at least 75%, at least 80%, at least 85%, at least 90%, at least 95%, or at least 99% identical to any one of SEQ ID NOs: 351-361 or 363. In some embodiments, the engineered therapeutic comprises an amino acid sequence that is at any one of SEQ ID NOs: 351-361 or 363. In some embodiments, the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 351-361 or 363. In some embodiments, the at least one oncoselective sequence comprises a nucleic acid motif. In some embodiments, the nucleic acid motif comprises an oncoselective readthrough motif. In some embodiments, the at least one oncoselective sequence comprises an untranslated region (UTR). In some embodiments, the UTR is a 5′ UTR or a 3′ UTR. In some embodiments, the at least one oncoselective sequence comprises a nucleic acid sequence that is at least 75%, at least 80%, at least 85%, at least 90%, at least 95%, or at least 99% identical to any SEQ ID NOs: 1-75, 91-97, or 101-146; or any one of SEQ ID NOs: 76-90. In some embodiments, the at least one oncoselective sequence comprises a nucleic acid sequence that is at least 50 contiguous nucleotides, at least 100 contiguous nucleotides, or at least 150 contiguous nucleotides identical to any one of SEQ ID NOs: 1-75, 91-97, or 101-146; or at least 5 contiguous nucleotides identical to any one of SEQ ID NOs: 76-90. In some embodiments, the engineered polynucleotide comprises two oncoselective sequences. In some embodiments, the two oncoselective sequences flank the coding sequence. In some embodiments, the at least one oncoselective sequence comprises at least one miRNA binding site. In some embodiments, the at least one oncoselective sequence comprises at least one protein binding site. In some embodiments, the at least one protein binding site is a RNA binding protein (RBP) site. In some embodiments, the engineered polynucleotide comprises at least one nucleic acid modification. In some embodiments, the at least one nucleic acid modification comprises a methylation. In some embodiments, the at least one nucleic acid modification comprises a pseudouridine. In some embodiments, the at least one nucleic acid modification comprises an N1-Methylpseudouridine. In some embodiments, the engineered therapeutic comprises an engineered cytokine comprising modified cytokine comprising a secreted cytokine, a membrane tethered cytokine, a masked cytokine, a cytokine fusion, or a combination thereof. In some embodiments, the cytokine fusion comprises a cytokine coupled to a half-life extension domain. In some embodiments, the half-life extension domain comprises an antibody or fragment thereof a serum binding protein or fragment thereof. In some embodiments, the engineered cytokine comprises an interleukin or an interferon. In some embodiments, the engineered cytokine comprises the interleukin. In some embodiments, the interleukin comprises an IL-2. In some embodiments, the interleukin comprises an IL-12. In some embodiments, the interleukin comprises an IL-15. In some embodiments, the interferon comprises an IFN-α. In some embodiments, the at least one oncoselective sequence comprises a secondary structure. In some embodiments, the secondary structure comprises a stem loop; a bulge loop, a pseudoknot, or a combination thereof. In some embodiments, the stem loop comprises a double stem loop. In some embodiments, the at least one oncoselective sequence comprises a truncation. In some embodiments, the engineered polynucleotide comprises RNA. In some embodiments, the engineered polynucleotide comprises reduced immunogenicity. In some embodiments, the at least one oncoselective sequence increases expression of the engineered therapeutic in a cancer cell compared to expression of the engineered therapeutic in a normal cell. In some embodiments, the disease or condition comprises solid tumor. In some embodiments, the disease or condition comprises cancer. In some embodiments, the cancer comprises melanoma, breast cancer, basal cell carcinoma (BCC), squamous cell carcinoma (SCC), cutaneous SCC (cSCC or CSCC), Head & Neck Cancer, Thyroid Cancer, Colorectal Cancer, Prostate Cancer, Liver cancer, Pancreatic Cancer, Renal Cell Carcinoma, Brain Cancer, Soft Tissue Sarcoma, Lung Cancer, or a combination thereof. In some embodiments, the cancer does not respond to immune checkpoint inhibitor treatment.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1 illustrates overlap in peptide identifications between multiple peptide search engines used in the proteomic pipeline.

FIG. 2 illustrates relative 3′ untranslated region (UTR) and 5′ UTR Peptide-Spectrum Matches (PSMs) counts in multi-tissue search results.

FIG. 3 illustrates the fold selectivity of alternative site of translation (aTIS) 5′ UTR elements tested in K562 cell line versus healthy BJ cell lines.

FIG. 4 illustrates translation efficiency of UTR elements in K562 cells versus healthy BJ cells.

FIG. 5 illustrates translation efficiency change of 5′ UTR elements relative to AML12 cell lines.

FIG. 6 illustrates raw nano-luciferase activity 48 hours following incubation with LNP-mRNA.

FIG. 7 illustrates translation selectivity of RNA sequences in MC38 compared with B16-F10 and AML12 by assay.

FIG. 8 illustrates B16-F10 cellular translation fold change relative to AML12 translation for tested RNA sequences.

FIG. 9 illustrates resulting fluorescence intensity over time of a non-selective mRNA (3′ UTR002, SEQ ID NO: 94) sequence.

FIG. 10 illustrates resulting fluorescence intensity over time of SEQ ID NO: 4 (3′ UTR152) mRNA sequence.

FIG. 11 illustrates the cellular translation measurement resulting from fluorescence AUC measurement over 144 hours across healthy BJ cells and K562 cells between non-selective mRNA (3′ UTR002, SEQ ID NO: 94) and SEQ ID NO: 4 (3′ UTR152).

FIG. 12 illustrates comparison of selective translation via fluorescence AUC over 144 hours of SEQ ID NO: 4 (3′ UTR152)-UTP and SEQ ID NO: 5 (3′ UTR152)-N1 pseudouridine versus 3′ UTR002-UTP and 3′ UTR002-N1 pseudouridine in BJ cells versus K562 cells.

FIG. 13 illustrates in vivo selective translation of FireFly Luciferase in B16-F10 tumor cells versus liver cells (n=5, bars represent f standard error of mean (SEM), ***p≤0.001).

FIG. 14 illustrates in vivo visualization of fluorescence regions in oncoselective versus non-selective RNA sequences.

FIG. 15 illustrates selective translation in human cancer cell lines versus human primary hepatocytes (HsHep; n=6, bars represent f standard deviation, ordinary two-way ANOVA, ns p>0.05, *** p<0.001, ****p<0.001).

FIG. 16 illustrates the result of RNA translation measured by luminescence assay in B16-F10 and AML12 cells of truncated SEQ ID NO: 5 (3′ UTR155).

FIG. 17 illustrates the result of RNA translation measured by luminescence assay in MC38 and AML12 cells of truncated SEQ ID NO: 5 (3′ UTR155).

FIG. 18 illustrates the result of RNA translation measured by luminescence assay in B16-F10 and AML12 cells of truncated SEQ ID NO: 5 (3′ UTR155).

FIG. 19 illustrates the result of RNA translation measured by luminescence assay in MC38 and AML12 cells of additional versions of truncated SEQ ID NO: 5 (3′ UTR155).

FIG. 20 illustrates the computational results across the broader library of mRNA sequences tested.

FIG. 21 illustrates the screening of mRNA sequence motifs for oncoselective translation.

FIG. 22 illustrates the fold of selectivity towards K562 cells from screening results of mRNA sequence motifs for oncoselective translation, including across unmodified UTP and modified UTP.

FIG. 23 illustrates the One-Glo raw luminescence assay results B16-F10 and MC-38 cell line translation compared with the healthy AML12 cell line.

FIG. 24 illustrates the fold change in translational selectivity of RNA sequences relative to B16-F10 and MC-38 cell lines.

FIG. 25 illustrates the nucleotide index in 3′-UTR HMGB2 alignment versus the sequence fraction matching human sequences.

FIG. 26 illustrates a plot of evolutionary conservation of bases to human sequences in the functional region of the sequence shows conserved motif sequence for oncoselective RNA.

FIG. 27A illustrates sequence motif alignment of 3′UTR sequence positions 2-9.

FIG. 27B illustrates sequence motif alignment of 3′UTR sequence positions 10-22.

FIG. 27C illustrates sequence motif alignment of 3′UTR sequence positions 23-29.

FIG. 27D illustrates sequence motif alignment of 3′UTR sequence positions 30-35.

FIG. 27E illustrates sequence motif alignment of 3′UTR sequence positions 48-56.

FIG. 28A illustrates sequence motif alignment of 3′UTR sequence positions 58-66.

FIG. 28B illustrates sequence motif alignment of 3′UTR sequence positions 141-145.

FIG. 28C illustrates sequence motif alignment of 3′UTR sequence positions 126-131.

FIG. 28D illustrates sequence motif alignment of 3′UTR sequence positions 108-112.

FIG. 28E illustrates sequence motif alignment of 3′UTR sequence positions 3047.

FIG. 28F illustrates sequence motif alignment of 3′UTR sequence positions 171-177.

FIG. 29A illustrates alignment of a larger motif sequences focused on a specific region.

FIG. 29B illustrates larger sequence motifs upon alignment for oncoselective RNA sequences.

FIG. 30 illustrates retention of selectivity in malignant cells based on RNA sequence (n=3, students t-test, ns=no significant change in selectivity vs parent UTR. Bars represent mean±standard deviation or SD).

FIG. 31 illustrates the fold of enhancement in translational efficiency over parent UTR. (n=3, students t-test, ns=no significant change in translational efficiency vs non-selective RNA sequence. Bars represent mean±standard deviation (SD).

FIG. 32A illustrates the translation selectivity of mRNA sequences tested within the respective cell lines tested. An ANOVA test was run comparing the mean selectivity scores of each UTR for B16-F10 vs AML12 and MC38 vs AML12 (n=3, *p-value>0.05, ** p-value>0.01, *** p-value>0.001).

FIG. 32B illustrates relative translation calculations, including percentage of Firefly luciferase (FLuc) signal relative to 3′-UTR002, the non-selective control sequence, of mRNA sequences tested relative to 3′ UTR002.

FIG. 32C illustrates raw FLuc signal based on cell lines.

FIG. 33 illustrates an exemplary secondary structure of an oncoselective sequence described herein.

FIG. 34 illustrates selective expression in cancer cells (MC38 or B16-F10).

FIG. 35A illustrates in vivo imaging of oncoselective sequence in MC38 tumor model.

FIG. 35B illustrates radiance and selectivity expression of the oncoselective sequence described herein.

FIG. 35C illustrates transfection (utilizing MessengerMAX transfection reagent) efficiency of the oncoselective sequences described herein (5UTR176 or SEQ ID NO: 140; 5UTR-177-5UTR182; or a combination of 5UTR025 or SEQ ID NO: 138 and 3UTR002 or SEQ ID NO: 94) as determined by luciferase relative light unit (RLU) in non-cancerous cell (human umbilical vein endothelial cell or HUVEC), myelogenous leukemia cell (K562), or non-small cell lung carcinoma cell (H1299).

FIG. 35D illustrates the selectivity of the oncoselective sequences in FIG. 35C for driving expression of the luciferase in cancer cell compared to non-cancerous cell.

FIG. 35E illustrates replicate 1 of peripheral LNP transfection of the oncoselective sequences described herein (3UTR155 or SEQ ID NO: 123; 3UTR578 or SEQ ID NO: 124; 3UTR579 or SEQ ID NO: 125; 3UTR319 or SEQ ID NO: 121; 3UTR583 or SEQ ID NO: 129; 3UTR584 or SEQ ID NO: 130; 3UTR585 or SEQ ID NO: 131; combination of 5UTR136 or SEQ ID NO: 139 and 3UTR318 or SEQ ID NO: 122; combination of 5UTR136 or SEQ ID NO: 139 and 3UTR580 or SEQ ID NO: 126; or 3UTR002 or SEQ ID NO: 94) in HUVEC versus K562 (biological replicate 1 with n=3 technical replicates).

FIG. 35F illustrates replicate 1 of the selectivity of the oncoselective sequences (as shown in FIG. 35E) described herein for driving expression of the luciferase in cancer cell compared to non-cancerous cell.

FIG. 35G illustrates replicate 2 of peripheral LNP transfection of the oncoselective sequences described herein (3UTR155 or SEQ ID NO: 123; 3UTR578 or SEQ ID NO: 124; 3UTR579 or SEQ ID NO: 125; 3UTR319 or SEQ ID NO: 121; 3UTR583 or SEQ ID NO: 129; 3UTR584 or SEQ ID NO: 130; 3UTR585 or SEQ ID NO: 131; combination of 5UTR136 or SEQ ID NO: 139 and 3UTR318 or SEQ ID NO: 122; combination of 5UTR136 or SEQ ID NO: 139 and 3UTR580 or SEQ ID NO: 126; or 3UTR002 or SEQ ID NO: 94) in HUVEC versus K562 (biological replicate 1 with n=3 technical replicates).

FIG. 35H illustrates replicate 2 of the selectivity of the oncoselective sequences (as shown in FIG. 35G) described herein for driving expression of the luciferase in cancer cell compared to non-cancerous cell.

FIG. 35I illustrates combination of replicate 1 (FIG. 35E) and replicate 2 (FIG. 35G) of peripheral LNP transfection of the oncoselective sequences described herein in HUVEC versus K562 (biological replicate 1 with n=3 technical replicates).

FIG. 35J illustrates combination of replicate 1 (FIG. 35F) and replicate 2 (FIG. 35H) of the selectivity of the oncoselective sequences described herein for driving expression of the luciferase in cancer cell compared to non-cancerous cell.

FIG. 35K illustrates replicate 1 of peripheral LNP transfection of the oncoselective sequences described herein (3UTR002 or SEQ ID NO: 94; 3UTR155 or SEQ ID NO: 123; 3UTR319 or SEQ ID NO: 121; 3UTR579 or SEQ ID NO: 125; 3UTR584 or SEQ ID NO: 130; 3UTR592 or SEQ ID NO: 132; 3UTR593 or SEQ ID NO: 133; 3UTR594 or SEQ ID NO: 135; 3UTR597 or SEQ ID NO: 137; 3UTR598 or SEQ ID NO: 134; or 3UTR596 or SEQ ID NO: 136) in HUVEC versus K562 (biological replicate 1 with n=3 technical replicates).

FIG. 35L illustrates replicate 1 of the selectivity of the oncoselective sequences (as shown in FIG. 35K) described herein for driving expression of the luciferase in cancer cell compared to non-cancerous cell.

FIG. 35M illustrates replicate 2 of peripheral LNP transfection of the oncoselective sequences described herein (3UTR002 or SEQ ID NO: 94; 3UTR155 or SEQ ID NO: 123; 3UTR319 or SEQ ID NO: 121; 3UTR579 or SEQ ID NO: 125; 3UTR584 or SEQ ID NO: 130; 3UTR592 or SEQ ID NO: 132; 3UTR593 or SEQ ID NO: 133; 3UTR594 or SEQ ID NO: 135; 3UTR597 or SEQ ID NO: 137; 3UTR598 or SEQ ID NO: 134; or 3UTR596 or SEQ ID NO: 136) in HUVEC versus K562 (biological replicate 1 with n=3 technical replicates).

FIG. 35N illustrates replicate 2 of the selectivity of the oncoselective sequences (as shown in FIG. 35M) described herein for driving expression of the luciferase in cancer cell compared to non-cancerous cell.

FIG. 35O illustrates combination of replicate 1 (FIG. 35K) and replicate 2 (FIG. 35M) of peripheral LNP transfection of the oncoselective sequences described herein in HUVEC versus K562 (biological replicate 1 with n=3 technical replicates).

FIG. 35P illustrates combination of replicate 1 (FIG. 35L) and replicate 2 (FIG. 35N) of the selectivity of the oncoselective sequences described herein for driving expression of the luciferase in cancer cell compared to non-cancerous cell.

FIG. 35Q illustrates replicate 1 of peripheral LNP transfection of the oncoselective sequences described herein (3UTR002 or SEQ ID NO: 94; 3UTR155 or SEQ ID NO: 123; 3UTR319 or SEQ ID NO: 121; 3UTR579 or SEQ ID NO: 125; 3UTR584 or SEQ ID NO: 130; 3UTR592 or SEQ ID NO: 132; 3UTR593 or SEQ ID NO: 133; 3UTR594 or SEQ ID NO: 135; 3UTR597 or SEQ ID NO: 137; 3UTR598 or SEQ ID NO: 134; or 3UTR596 or SEQ ID NO: 136) in MC38 (colon adenocarcinoma cell) versus AML12 (non-cancerous hepatocyte cell) (biological replicate 1 with n=3 technical replicates).

FIG. 35R illustrates replicate 1 of the selectivity of the oncoselective sequences (as shown in FIG. 35Q) described herein for driving expression of the luciferase in cancer cell compared to non-cancerous cell.

FIG. 35S illustrates replicate 2 of peripheral LNP transfection of the oncoselective sequences described herein (3UTR002 or SEQ ID NO: 94; 3UTR155 or SEQ ID NO: 123; 3UTR319 or SEQ ID NO: 121; 3UTR579 or SEQ ID NO: 125; 3UTR584 or SEQ ID NO: 130; 3UTR592 or SEQ ID NO: 132; 3UTR593 or SEQ ID NO: 133; 3UTR594 or SEQ ID NO: 135; 3UTR597 or SEQ ID NO: 137; 3UTR598 or SEQ ID NO: 134; or 3UTR596 or SEQ ID NO: 136) herein in MC38 versus AML12 (biological replicate 1 with n=3 technical replicates).

FIG. 35T illustrates replicate 2 of the selectivity of the oncoselective sequences (as shown in FIG. 35S) described herein for driving expression of the luciferase in cancer cell compared to non-cancerous cell.

FIG. 35U illustrates combination of replicate 1 (FIG. 35Q) and replicate 2 (FIG. 35S) of peripheral LNP transfection of the oncoselective sequences described herein in HUVEC versus K562 (biological replicate 1 with n=3 technical replicates).

FIG. 35V illustrates combination of replicate 1 (FIG. 35R) and replicate 2 (FIG. 35T) of the selectivity of the oncoselective sequences described herein for driving expression of the luciferase in cancer cell compared to non-cancerous cell.

FIG. 36 illustrates treatment of subcutaneous B16-F10 melanoma tumors with LNPs comprising therapeutic cocktail.

FIG. 37 illustrates overall survival after treatment with the engineered therapeutic cocktail and a control mRNA.

FIG. 38 illustrates dose titration treatment of subcutaneous B16-F10 melanoma tumors with LNPs comprising therapeutic cocktail.

FIGS. 39A-C illustrate dose-response relationship of therapeutic cocktail in B16-F10 melanoma.

FIG. 40 illustrates individual tumor growth curves of dose titration treatment of subcutaneous B16-F10 melanoma tumors with LNPs comprising therapeutic cocktail as shown in FIGS. 39A-C.

FIG. 41 illustrates efficacy evaluation in Pan02 pancreatic adenocarcinoma.

FIG. 42A illustrates individual Pan02 tumor growth curves.

FIG. 42B illustrates survival proportions upon therapeutic cocktail treatment.

FIG. 43 illustrates efficacy evaluation in YUMM1.7 melanoma.

FIG. 44A illustrates individual YUMM1.7 tumor growth curves.

FIG. 44B illustrates survival proportions upon therapeutic cocktail treatment.

FIGS. 45A-C illustrate efficacy evaluation in MC38.K tumors.

FIG. 46 illustrates individual MC38.K tumor growth curves.

FIG. 47 illustrates individual tumor growth curves after implant with 3×10⁵ MC38.K cells in C57BL/6 mice.

FIGS. 48A-B illustrate tolerability of intratumoral therapeutic cocktail mRNA therapy.

FIGS. 49A-C illustrate tolerability of systemic LNP administration.

FIGS. 50A-B illustrate K489-001 systemic Administration of oncoselective therapeutic.

FIG. 51 illustrates schematic representation of masked single-chain interleukin (IL) cytokine constructs.

FIG. 52 illustrates schematic representation of masked two-chain IL cytokine constructs.

FIG. 53 illustrates EC₅₀ values of masked and protease-treated (MMP) cytokine constructs in a HEK-Blue IL-12 cell reporter assay.

FIG. 54A-B illustrate the design process for the masked cytokine.

FIG. 54C illustrates construct mRNAs transfected into HEK293t cells using Lipofectamine Messenger Max. Supernatants were collected in 24 hours and concentrated. 3 ul of sample was used to run SDS-PAGE. All chimeric proteins show additional bands which might represent cleaved proteins.

FIG. 54D illustrates translation of hIL-2 entities A to M from mRNAs. SDS-PAGE and WB analysis of in vitro translated hIL-2 entities stained with anti-hIL-2 mAb (CS mRNA encoding free human IL-2. Proteases present in HEK cell culture caused partial linker cleavage for constructs D, E, F and G.

FIG. 54E illustrates masking efficiency evaluation by determining EC50 values in a functional assay for hIL-2 entities using HEK-Blue-IL-2 reporter cell line. The activation of the JAK-STAT pathway was monitored as a dose-response to hIL-2 protein entities translated from mRNAs A-M. Cells were stimulated with increasing concentrations. After overnight incubation, the STAT5 response was determined using QUANTI-Blue Solution, a SEAP detection reagent, and reading the optical density (OD) at 650 nm. N=2 (technical replicates), Mean±SD. Unmasked construct L was aligned with the standard free recombinant hIL-2. Constructs H and K had the highest EC50.

FIG. 54F illustrates percentage of intact (uncleaved) protein in HEK cell supernatant for hIL-2 entities determined by WB analysis. For the entities a high level of protein integrity and low level of linker, cleavage was detected. Integrity was determined for each entity with 2 to 4 transfections with mRNAs (table). Mean+/−SD was shown.

FIG. 55A-B illustrates masking efficiency evaluation by determining EC50 values in a functional assay for hIL-2 entities using HEK-Blue-IL-2 reporter cell line. Activation of IL-2 signaling was observed in presence of increasing concentrations of IL-2 entities. FIG. 55 A. Mean±SD of two to three different transfection reactions for each entity. FIG. 55B. Masking efficiency as fold EC50 increase relative to Pro065 (Mean t SD).

FIG. 56A illustrates comparison of functionality and masking efficiency of hIL-2 Pro061 from supernatants of different mRNA batches vs purified protein (Protein A followed by size exclusion columns; Genscript Biotech). Activity and masking efficiency of Pro061 produced from mRNAs correlated with the plasmid based Pro061 purified at GenScript.

FIG. 56B illustrates validation that linkers in IL-2 entities were cleavable by incubation with activated MMP-2 protease. Percentage of cleaved protein was determined using WB. Pro054, Pro055, Pro061, and Pro064 were sensitive to MMP-2 digestion and protease-dependent removal of masking and half-life extension domains.

FIG. 57A illustrates IL-12 cleavable protein entities produced in vitro from their respective mRNAs. (reducing SDS-PAGE). HEK293t cells were transfected with RNA using Lipofectamine MessengerMAX. The protein weights and integrity was analyzed using quantitative Western Blot. Membranes were developed with anti-IL-12B (p40) mAb to detect PRO071 or PR0073. Predicted MW for PRO071 is 69 kDa and 108 kDa for PR0073. Proteins may run higher due to glycosylation.

FIG. 57B illustrates mIL-12 non-cleavable protein entities produced in vitro from their respective mRNAs. (reducing SDS-PAGE). HEK293t cells were transfected with RNA using Lipofectamin MessengerMAX Transfection Reagent. The protein weights and integrity was analyzed using quantitative Western Blot. Membranes were stained with anti-IL-12B mAb to detect Pro075 and Pro077. Predicted MW for Pro075 was 65 kDa, and for Pro077 was 100 kDa. Proteins could run higher due to glycosylation.

FIG. 58 illustrates masking efficiency evaluation by determining EC50 values in a functional assay for cleavable hIL-12 entities using HEK-Blue-IL-12 reporter cell line. Activation of IL-12 signaling was observed in presence of increasing concentrations of IL-12 entities. Mean±SD of two to three different transfection reactions for each entity.

FIG. 59 illustrates masking efficiency evaluation by determining EC50 values in a functional assay for non-cleavable hIL-12 entities using HEK-Blue-IL-12 reporter cell line. Activation of IL-12 signaling was observed in presence of increasing concentrations of IL-12 entities. Mean±SD of two to three different transfection reactions for each entity.

FIG. 60 illustrates masking efficiency as fold EC50 increase relative to Mil-12 protein standard (Mean±SD).

FIG. 61 illustrates mIL-15 protein entities produced in vitro from their respective mRNAs. Predicted sizes in kDa were provided. (glycosylated, reducing SDS-PAGE). HEK293t cells were transfected with RNA using Lipofectamin MessengerMAX Transfection Reagent. The protein weights and integrity was analyzed using quantitative Western Blot.

FIG. 62 illustrates percentage of total intact (e.g., uncleaved) protein of mIL-15 entities in supernatant. For each individual IL-15 entity, results from 3 mRNA transfections were shown.

FIG. 63 illustrates cell survival after addition of different concentrations of mIL-15 entities. N=2, technical replicates. Mean±SD. Using CTLL2 cells, cells were preincubated in cell media with low IL-2. Then, samples were applied and left to incubate for 3 days. Before that, samples were preincubated with 50% human serum for 30 min. Cell survival was measured using Promega Substrate Cell Titer 96 Aqueous One Solution Reagent. For each individual IL-15 entity results from 3 mRNA transfections are shown.

FIG. 64 illustrates fold change of EC50 for IL-15 entities calculated relative to unmasked entity (PRO045). EC50s were calculated using the functional CTLL2 assay in FIG. 63. Mean+/−SD of 3 transfection replicates with mRNAs are shown. Treatment with Pro101 led to higher EC50 fold change relative to Pro045 compared to Pro102. Despite partial cleavage observed in Pro101 and no cleavage in Pro102. Pro104 and Pro101 (both contain 2 HSA binding domains) demonstrated the highest EC50 fold change.

FIG. 65A illustrates protease-dependent cleavage. SDS-PAGE WB results for mIL-15 entities after digestion with MMP2 to validate cleavability of the linkers. Pro104 served as a control with a non-cleavable linker. Fully cleaved product was expected at ˜25 kDa but ran higher mainly due to glycosylation.

FIG. 65B illustrates effective linker cleavage in Pro101. Fraction of cleaved Pro101 following MMP2 protease treatment was determined by WB.

FIG. 66 illustrates human IL-15 protein entities produced from their respective mRNAs. Predicted sizes in kDa were provided. (glycosylated, reducing SDS-PAGE). SDS-PAGE results for lead mIL-15 candidates were digested with MMP2 to test cleavability of the linkers. Proteins were produced by GenScript. R—reducing conditions; NR—reducing conditions. For the functional assay, HEK-Blue-IL-2 cells were used. Samples were incubated with human serum for 30 min at 37° C. before use.

FIG. 67 illustrates IL-2 pathway activation in presence of hIL-15 entities produced from RNA or as a purified protein (size exchange chromatography).

FIG. 68 illustrates hIL-15 protein entities produced in vitro from their respective mRNAs. Predicted sizes in kDa were provided. (glycosylated, reducing SDS-PAGE). HEK293t cells were transfected with RNA using Lipofectamin MessengerMAX Transfection Reagent. The protein weights and integrity was analyzed using quantitative Western Blot.

FIG. 69 illustrates protease-dependent cleavage. SDS-PAGE WB results for hIL-15 entities after digestion with MMP2 were shown to validate cleavability of the linkers. Fully cleaved product was expected at ˜25 kDa, but runs higher mainly due to glycosylation.

FIG. 70 illustrates comparison of masking efficiency of hIL-15 entities before and after digestion to demonstrate that protein functionality (e.g., ability to activate IL-2 signaling) was preserved after the digestion.

FIG. 71 illustrates percentage of total intact (uncleaved) mIFN-α entities in supernatant.

FIG. 72 illustrates activation of mIFN-α signaling after treatment with increasing concentrations of different mIFNa entities. Activity of in-house produced Pro061 correlated with the Pro061 purified by GenScript, which supported validity of developed qWB.

FIG. 73 illustrates protease-dependent cleavage. SDS-PAGE WB results for mINF-α entities after digestion with MMP2 to validate cleavability of the linkers were shown. Fully cleaved product was expected at ˜25 kDa but ran higher mainly due to glycosylation. Pro104 served as a control with a non-cleavable linker.

FIG. 74 illustrates comparison of hIFN-α signaling activation after in presence of hIFN-α entities produced from RNA before and after digestion to assess masking efficiency and demonstrated that protein functionality (e.g., ability to activate IFN-α signaling) was preserved after the digestion.

FIG. 75A illustrates comparison of purified Pro084 (His-tag+SEC) hIFN-α and unmasked hIFN-α (protein standard).

FIG. 75B illustrates SDS-PAGE result for purified Pro084.

FIG. 76 illustrates percentage of digested protein determined using Wester blotting (WB).

FIG. 77 illustrates comparison of masking efficiency of hIFN-α entities before and after digestion to demonstrate that protein functionality (e.g., ability to activate IFNa signaling) was preserved after the digestion. EC50 fold change between MMP+ and MMP− was about 10 times. It was two times lower than for native samples.

FIG. 78A illustrates evaluation of IFN-γ pharmacodynamic responses (downstream immune activation of mKR-336) to the treatment. Tumor selectively increased IFNgamma over a 20-fold dose range (0.05-1 mg/Kg). Inflammatory milieu induced in tumor (MSD assay of tumor lysates). Dose titration of systemic KR-336/IL-12 mRNA-LNPs for evaluating single-chain masking effect was performed by measuring IFNgamma levels in various organs 48 hours after dosing. Standard mRNA-LNPs were administered IV in C57BL/6J female mice carrying a subcutaneous MC38.K tumors (n=5/group). Unmasked control was encoded by mRNA-LNP in group 4.

FIG. 78B illustrates IFNgamma levels in serum following dosing.

FIG. 78C illustrates serum IFNgamma 48 hours post-dose 2. “Native” dose at 0.25 mg/kg was a not tolerated control (>MTD) and induced toxic IFNγ levels and shown as standard in all three panels. ANOVA statistical analysis. By comparison, treatment with masked entity was tolerated (IFNgamma level, body weight, overall body score) demonstrating an increase in biological tolerability and therapeutic index through masking.

FIG. 78D illustrates IFNgamma levels in organ lysates normalized to total protein content (BCA assay based). mKR-336 induced strong IFNy Levels in the tumor while avoiding toxic levels in the liver.

FIG. 78E illustrates oncoselectivity of mKR-336. Graph showed the tumor:liver ratio of IFNgamma levels following treatment.

FIG. 79A-C illustrates efficacy of KR-335/336 when administered systemically. Masking of mKR-335 (Pro064, Pro072, Pro073, Pro101, or Pro093) and mKR-336 (Pro072 or Pro073) for was tested for tolerability and anti-tumor efficacy efficiency vs unmasked control (NS-335: Pro065, Pro037, Pro045, or Pro094, NS-336: Pro070 or Pro071) in the MC38.K C57BL/6 model. Peripheral mRNA-LNPs were employed and administered IV for systemic administration or IT for comparison with direct tumor application. FIG. 79A illustrates mKR-335 was tolerated when administered IV, while NS-335 was not. FIG. 79B illustrates mKR-335 provided TGI benefit. FIG. 79C illustrates overall survival benefit (tumor burden limit at 1400 mm³).

FIG. 80A-C illustrates efficacy of KR-336 when administered systemically. The masking of mKR-336 (Pro072 or Pro073) was tested for tolerability and anti-tumor efficacy efficiency vs unmasked control (NS-336: Pro070 or Pro071) in the MC38.K C57BL/6 model. Peripheral mRNA-LNPs were employed and administered intravenously (IV) for systemic administration or intratumorally (IT) for comparison with direct tumor application. FIG. 80A illustrates mKR-336 was tolerated when administered IV, while NS-336 was not. FIG. 80B illustrates mKR-336 providing TGI benefit. FIG. 80C illustrates overall survival benefit (tumor burden limit at 1400 mm³).

FIG. 81A-B illustrates efficacy of systemic administration of mKR-336 in MC38 tumor bearing C57BL/6 mice for determining if masking and half-life extension improved the therapeutic window and test for high tolerability of peripheral version with 4 LNP based mRNA payloads after repeated dosing. Comparison of mKR-336 at 0.145 mg/kg to NT B-actin Control was performed at 0.3 mg/Kg administered via tail vein injection. FIG. 81A illustrates mKR-336 systemic treatment resulted in tumor growth inhibition and completed responses, while being well tolerated. FIG. 81B illustrates mKR-336 with peripheral LNP V4 causing no body weight loss and providing a wide therapeutic window that allowed prolonged dose schedule. mKR-336 provided an overall survival benefit.

FIG. 82A illustrates body weight change of the three group of animals with the animals dosed on day 0, day 7, day 14, or day 21 of the study. On day 16, significant body weight difference was observed in hEPO treated group versus the KR-335 treated group (p=0.04; 2-way ANOVA). No significant body weight change was observed between groups on all other timepoints.

FIG. 82B illustrates body weight change of the individual animals with the animals dosed on day 0, day 7, day 14, or day 21 of the study.

FIG. 83A illustrates aspartate aminotransferase (AST) measurement of the three group of animals during the study. No significant AST differences were observed based on all 2-way ANOVA group comparison.

FIG. 83B illustrates aspartate aminotransferase (AST) measurement of the individual animals during the study.

FIG. 84A illustrates alanine transaminase (ALT) measurement of the three group of animals during the study. No significant ALT differences were observed based on all 2-way ANOVA group comparison.

FIG. 84B illustrates alanine transaminase (ALT) measurement of the individual animals during the study.

FIG. 85A illustrates alkaline phosphatase (ALP) measurement of the three group of animals during the study. No significant ALP differences were observed based on all 2-way ANOVA group comparison.

FIG. 85B illustrates alkaline phosphatase (ALP) measurement of the individual animals during the study.

FIG. 86A illustrates gamma-glutamyl transpeptidase (GGT) measurement of the three group of animals during the study. No significant GGT differences were observed based on all 2-way ANOVA group comparison.

FIG. 86B illustrates gamma-glutamyl transpeptidase (GGT) measurement of the individual animals during the study.

FIG. 87A illustrates white blood cell (WBC) count of the three group of animals during the study. No significant WBC count differences were observed based on all 2-way ANOVA group comparison. FIG. 87B illustrates white blood cell (WBC) measurement of the individual animals during the study.

### DETAILED DESCRIPTION

In view of the difficulties for combating cancer, there remains an ongoing need for a composition or method for treating cancer in an effective manner, where the treatment decreases the progression of cancer, decreases presence of cancer or cancer cells, or increases response of cancer compared to cancer treatment modalities that are currently available. There is also a need to limit the delivery or activity of therapeutic to cancer cells. Absence of such specificity, cancer therapeutic can lead to adverse side effects. Additionally, there is a need for treatment option with increased half-life and improved pharmacokinetics.

Through the combination of lipid nanoparticle design with selectively translated mRNA, described herein is oncoselective sequences for driving expression (e.g., expression of mRNA molecules) for differential translation in targeted tissues and cell types. By enabling selective translation in tumorigenic cells and tissues, the oncoselective sequences can avoid off-tumor translation in tissues such as the liver or spleen. The oncoselective sequences can be determined by various omics datasets (e.g., protein or mRNA ratio analysis). Cancer cells are aberrant compared to their matched healthy counterparts and tend to display different ribosome biogenesis and recruitment along with altered RNA and protein expression, stability, and structure. As such, putative mechanisms for oncoselective translation can be identified by screening naturally occurring nucleotide sequences for these possible mechanisms in both cancer cells and their healthy counterparts. Using structural analysis, these oncoselective sequences can be further optimized to be compatible for translation in cancer cells compared to healthy cells in combination with an engineered therapeutic payload. The expression specificity of these oncoselective sequences can be verified by high throughput screening such as a two-layer in vitro approach for evaluation.

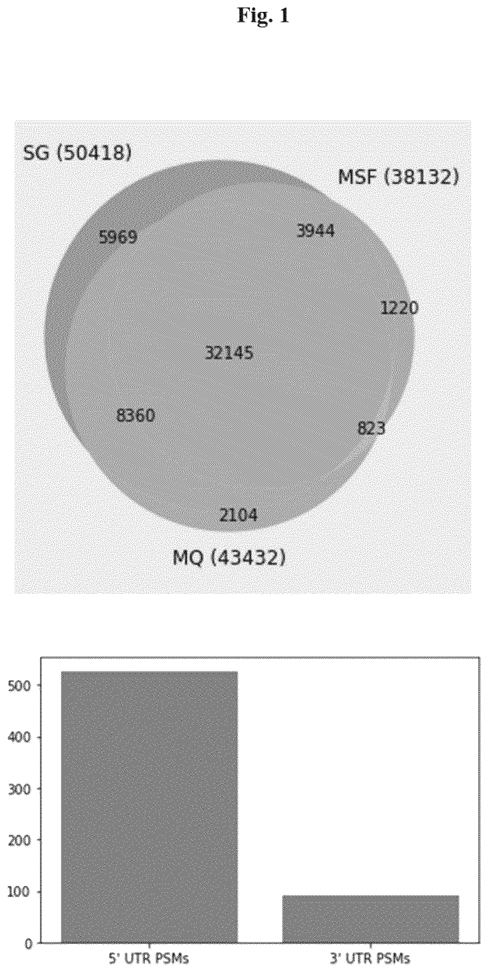

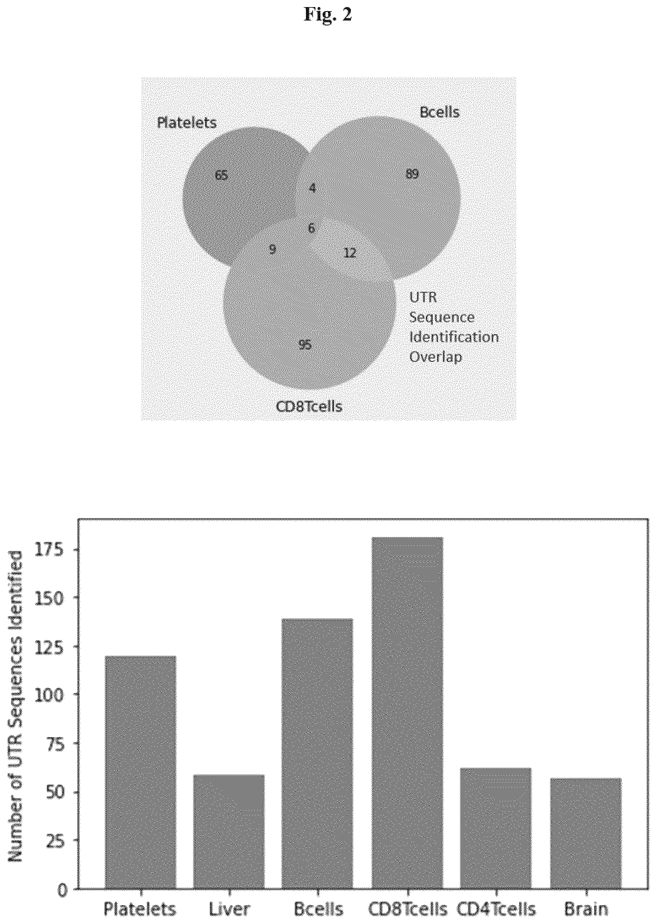

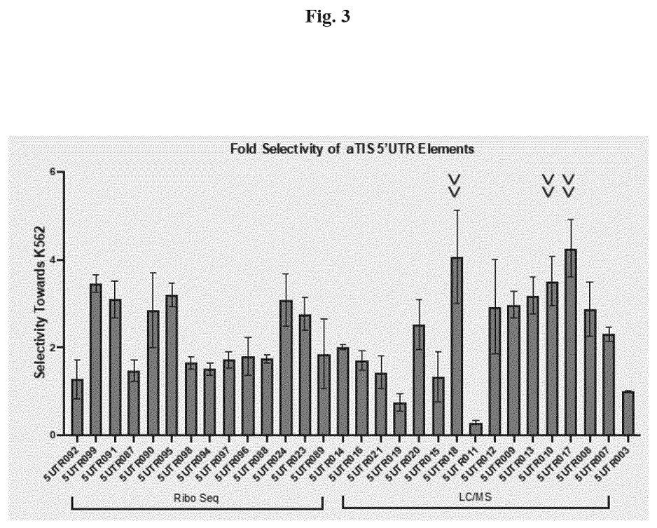

Described herein, in some aspects, are engineered polynucleotides comprising at least one oncoselective sequence, where the at least one oncoselective sequence on increase expression of a coding sequence in a cancer cell compared to expression of the same coding sequence in a normal cell. The oncoselective sequence can be identified based on analysis of non-canonical translation such as ribosome profiling or proteomics based on liquid chromatography and mass spectrometry (LC-MS). The non-canonical translation analysis can also include analyzing alternative translation initiation sites (aTIS) or stop codon readthroughs. Proteomics can include examining peptide abundance in samples and databases as shown in FIG. 1 and FIG. 2. Additional ribosome profiling can include ribosome profiling and sequencing (Ribo-Seq), determining abundance of peptide or messenger RNA (mRNA), RNA binding partner (e.g., miRNA or protein), or a combination thereof. The screening of the aforementioned methodologies can identify oncoselective sequence candidates, which can then be validated for increasing expression in cancer cells (e.g., FIGS. 3-6 and FIGS. 20-24). In some embodiments, the screening and validation can identify an oncoselective sequence, where the oncoselective sequence can comprise an oncoselective nucleic acid motif (FIGS. 26-29).

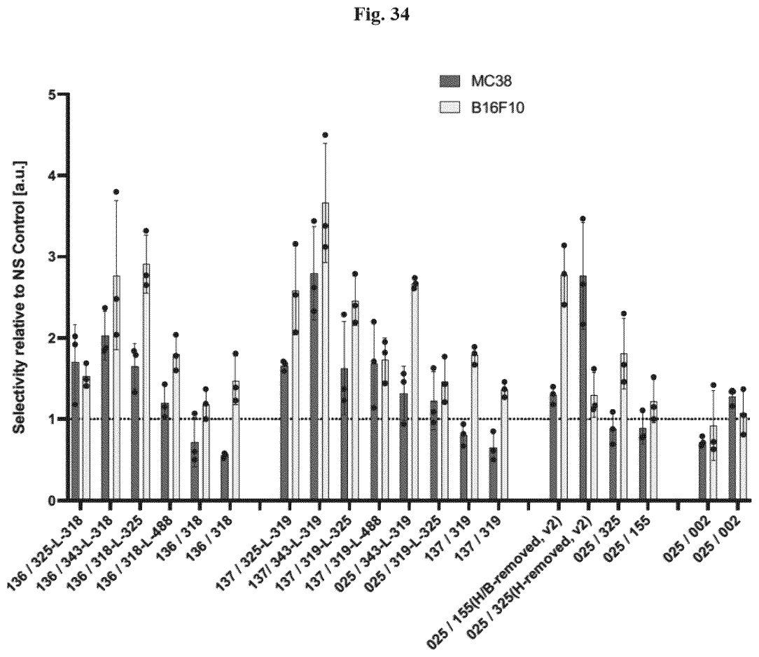

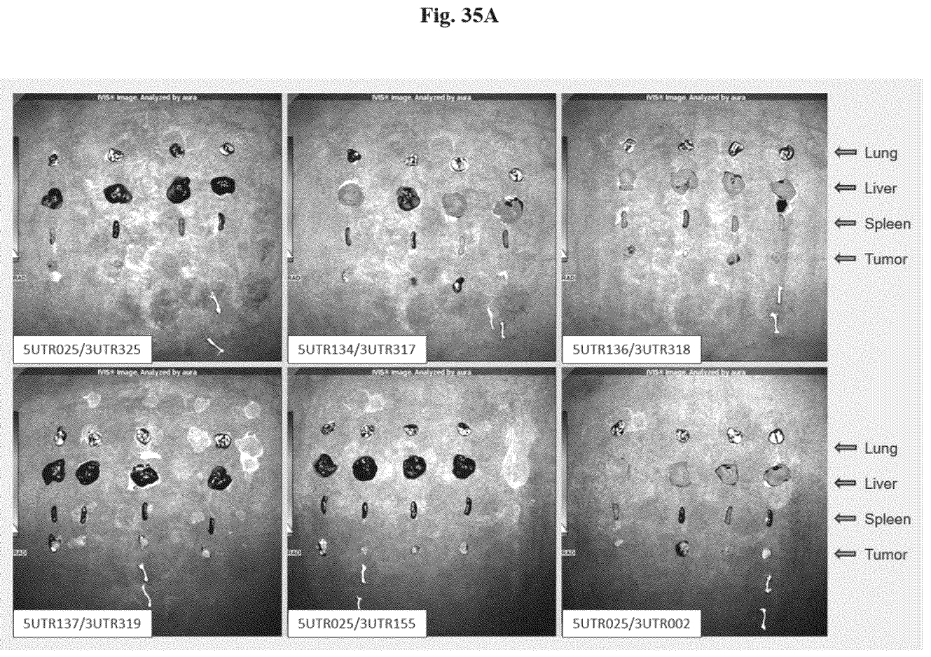

A range from 8-fold to 40-fold of oncoselectivity in solid tumors or and leukemia cell models can be obtained from these oncoselective sequences. Additionally, in vivo murine models show 10-fold higher translation in melanoma models than in healthy tissue. FIG. 34 illustrates such oncoselectivity, where oncoselective sequences (136-318 and 137-319) exhibit increased selectivity of expression compared to control sequences. 325-343-488 denote oncoselective sequences based on truncations of sequences of untranslated regions. FIG. 34 further illustrates combination of various 5′ or 3′ untranslated region (UTR) to arrive at oncoselective sequences for increased selective expression in the cancer cells. Examples of the oncoselective sequences can be found in SEQ ID NOs: 1-97 or 101-146.

In some embodiments, described herein is, an engineered polynucleotide comprising: at least one oncoselective sequence; and a coding sequence encoding an engineered therapeutic, wherein the at least one oncoselective sequence comprises a nucleic acid sequence that is: at least 75% identical to any one of SEQ ID NOs: 1-97 or SEQ ID NOs: 101-146. In some embodiments, the at least one oncoselective sequence comprises the nucleic acid sequence that is at least 75% identical to any one of SEQ ID NOs: 1-8. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-97 or SEQ ID NOs: 101-146. In some embodiments, the at least one oncoselective sequence comprises the nucleic acid sequence that is at least 50 contiguous nucleotides identical to any one of SEQ ID NOs: 1-8. In some embodiments, the at least one oncoselective sequence comprises the nucleic acid sequence that is at least 5 contiguous nucleotides identical to any one of SEQ ID NOs: 76-90. In some embodiments, the at least one oncoselective sequence comprises the nucleic acid sequence that is any one of SEQ ID NOs: 76-90. In some embodiments, the at least one oncoselective sequence comprises an untranslated region (UTR). In some embodiments, the engineered polynucleotide comprises two oncoselective sequences. In some embodiments, the engineered polynucleotide of further comprises at least one nucleic acid modification. In some embodiments, the at least one nucleic acid modification comprises a methylation. In some embodiments, the at least one nucleic acid modification comprises a pseudouridine. In some embodiments, the at least one nucleic acid modification comprises an NI-Methylpseudouridine.

In some embodiments, the engineered polynucleotide can be formulated into a composition. In some embodiments, the composition can further comprise a lipid such as a lipid nanoparticle (LNP). In some embodiments, the composition comprises two or more engineered polynucleotide described herein. For example, the composition can include at least two engineered polynucleotides, where each engineered polynucleotide encodes a different engineered therapeutic. In some embodiments, the composition comprises at least one additional polynucleotide for encoding an engineered therapeutic. For example, the composition can include a mixed population of engineered polynucleotides for encoding a mixture of engineered therapeutics. In some embodiments, the composition comprises a mixed population of engineered polynucleotides and polynucleotides for encoding at least one engineered therapeutic comprising an engineered interleukin or fragment thereof; or an engineered subunit of an interleukin. In some embodiments, the composition comprises an engineered polynucleotide encoding an engineered IL-12 or fragment thereof or an engineered subunit of IL-12. In some embodiments, the composition comprises an engineered polynucleotide encoding an engineered IL-2 or fragment thereof or an engineered subunit of IL-2. In some embodiments, the composition comprises at least one engineered polynucleotide encoding an engineered IL-12 or fragment thereof or an engineered subunit of IL-12 and at least one other therapeutic such as another cytokine. In some embodiments, the composition comprises at least one engineered polynucleotide encoding an engineered IL-2 or fragment thereof or an engineered subunit of IL-2 and at least one other therapeutic such as another cytokine.

In some embodiments, described herein are methods for treating a disease or condition with the engineered polynucleotide described herein. In some embodiments, the method comprises administering the engineered polynucleotide to a subject having the disease or condition. In some embodiments, the method comprises contacting the engineered polynucleotide with a lipid to form a composition described herein and subsequently administering the composition to the subject. In some embodiments, the method treats a disease or condition that is solid tumor. In some embodiments, the method treats disease or condition that is cancer. In some embodiments, the method comprises increasing expression of the engineered therapeutic described herein in a cancer cell compared to expression of the same engineered therapeutic in a normal cell. In some embodiments, the expression of the engineered therapeutic is increased by contacting a coding sequence encoding the engineered therapeutic with an oncoselective sequence described herein. In some embodiments, activity of the engineered therapeutic is increased in a cancer cell compared to activity of the engineered therapeutic in a normal cell. For example, the engineered therapeutic can include a masking domain, where the masking domain inhibits or decreases enzymatic activity of the engineered therapeutic. A cancer cell-specific protease can cleave the masking domain from the engineered therapeutic, thus activating the engineered therapeutic in a cancer cell as opposed to a normal cell. In some embodiments, the combination of oncoselective expression of the engineered therapeutic in a cancer cell and the activation of the engineered therapeutic in the same cancer cell synergistically increases the effectiveness of treating and killing the cancer cell.

In some aspects, the composition or method for treating cancer described herein comprises an engineered polynucleotide combination, where the engineered polynucleotide encodes at least one, at least two, at least three, or at least four polypeptides for treating the cancer. In some aspects, the engineered polynucleotide can include an mRNA. For example, the engineered polynucleotide can increase expression of an engineered therapeutic such as an interleukin or interferon for treating cancer in an oncoselective manner (e.g., increased expression of the polypeptide is only present in a tumor cell as opposed to a normal, healthy cell). In some aspects, the composition or method for treating cancer described herein utilizes an mRNA combination, where the mRNA combination increases the effectiveness of treating cancer. In some embodiments, the engineered polynucleotide combination, while increasing the effectiveness of treating cancer, does not lead to increased deleterious effects. Additionally, the engineered polynucleotide combination presents a solution to an ongoing need for treating cancer, where the cancer is unresponsive to other treatment (e.g., cancer or tumor also known as immunologically “cold” that is resistant to conventional treatments).

In some aspects, the composition or method for treating cancer described herein comprises an engineered polynucleotide combination encoding at least one polypeptide comprising a cytokine or an interferon. In some aspects, the at least polypeptide comprises a cytokine that is modified. For example, the cytokine encoded by the engineered polynucleotide combination can include an IL-12, where the IL-12 can be secreted IL-12, membrane tethered IL-12, masked IL-112, Fc-fusion IL-12, sushi IL-12, or a combination thereof. In some embodiments, the mRNA combination encoded at least one cytokine or at least one interferon. For example, the mRNA combination can encode IL-2, IL-12, IL-15, or IFN-α.

Described herein, in some aspects, is an engineered polynucleotide comprising a coding sequence encoding an engineered therapeutic. In some embodiments, the engineered therapeutic is an engineered cytokine. In some embodiments, the engineered cytokine is an engineered interleukin or fragment thereof or an engineered interferon or fragment thereof. In some embodiments, the engineered cytokine is an engineered submit of an interleukin. In some embodiments, the engineered therapeutic comprises an amino acid sequence that is at least 75% identical to any one of SEQ ID NOs: 301-303, 311-314, 321-328, 331-335, 341-344, 483-487, or 648-663. In some embodiments, the engineered therapeutic comprises an amino acid sequence that is at least 50 contiguous amino acids identical to any one of SEQ ID NOs: 301-303, 311-314, 321-328, 331-335, 341-344, 483-487, or 648-663.

In some embodiments, described herein is an engineered polynucleotide comprising an oncoselective sequence. In some embodiments, the oncoselective sequence increases expression of the engineered therapeutic encoded by the coding sequence in a cancer cell compared to expression of the engineered therapeutic in a normal cell. In some embodiments, the at least one oncoselective sequence does not increase expression of the engineered therapeutic in a liver cell. In some embodiments, the at least one oncoselective sequence decreases expression of the engineered therapeutic in a normal cell. In some embodiments, the at least one oncoselective sequence decreases expression of the engineered therapeutic in a liver cell. In some embodiments, the oncoselective sequence comprises nucleic acid motif. In some embodiments, the oncoselective sequence comprises at least one secondary structure. In some embodiments, the oncoselective sequence comprises RNA. In some embodiments, the oncoselective sequence comprises an untranslated region. In some embodiments, the oncoselective sequence comprises RNA and increases expression of a coding sequence comprising mRNA. In some embodiments, the oncoselective sequence comprises a combination of two or more UTR sequences. In some embodiments, the two or more UTR sequences can be connected by a linker. (e.g., the linker sequence comprises a nucleic acid sequence of SEQ ID NO: 171: AAAACAACAACAACAACAACTCCAACTCAC).

In some aspects, described herein is an engineered polynucleotide comprising an oncoselective modification. In some embodiments, the oncoselective modification comprises an oncoselective sequence motif comprising an oncoselective readthrough motif, a 5′ UTR, a 3′ UTR, or a combination thereof. In some embodiments, the oncoselective modification comprises a nucleic acid sequence that is at least 75% identical to of any one of the nucleic acid sequences of SEQ ID NOs: 1-97 or 101-146.

### Composition

Described herein, in some aspects, is a composition comprising at least one engineered polynucleotide. In some embodiments, the at least one engineered polynucleotide encodes at least one therapeutic. In some embodiments, the at least one engineered polynucleotide encodes at least one engineered therapeutic. In some embodiments, the at least one engineered polynucleotide comprises at least one oncoselective modification described herein (e.g., oncoselective sequence, oncoselective translation, oncoselective ribosome, or a combination thereof), where the at least one oncoselective modification increases expression of the at least one engineered polynucleotide in a specific cell. For example, the oncoselective modification can increase expression of the at least one therapeutic encoded by the engineered polynucleotide in a cancer cell as opposed to expression of the at least one therapeutic in a cell (e.g., non-cancerous or health cell). Such oncoselective modification can be particularly useful for treating a disease or condition, where the at least one therapeutic exerts toxic side effects, and such toxic side effects should be restricted to the cell afflicted by the disease or condition. In some embodiments, the disease or condition is cancer, and the specific cell where increased expression induced by the at least one oncoselective modification is restricted is a cancer cell.

In some embodiments, the engineered polynucleotide comprises an oncoselective sequence. In some embodiments, the oncoselective sequence comprises a nucleic acid sequence that is: at least 75% identical to any one of SEQ ID NOs: 1-97 or 101-146. In some embodiments, the oncoselective sequence comprises the nucleic acid sequence that is any one of SEQ ID NOs: 1-97 or 101-146. In some embodiments, the oncoselective sequence comprises the nucleic acid sequence that is at least 50 contiguous nucleotides, at least 100 contiguous nucleotides, or at least 150 contiguous nucleotides identical to any one SEQ ID NOs: 1-75, 91-97, or 101-146. In some embodiments, the oncoselective sequence comprises the nucleic acid sequence that is at least 5 contiguous nucleotides identical to any one of SEQ ID NOs: 76-90.

In some embodiments, the engineered polynucleotide encodes at least one therapeutic. In some embodiments, the at least one therapeutic comprises at least one cytokine. Non-example of the cytokine can include 4-1BBL, acylation stimulating protein, adipokine, albinterferon, APRIL, Arh, BAFF, Bcl-6, CCL1, CCL1/TCA3, CCL11, CCL12/MCP-5, CCL13/MCP-4, CCL14, CCL15, CCL16, CCL17/TARC, CCL18, CCL19, CCL2, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CCR10, CCR3, CCR4, CCR5, CCR6, CCR7, CCR8, CD153, CD154, CD178, CD40LG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL11, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAMI9A3, FAM19A4, FAMI9A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFN A5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17 family, IL-17A-F, IL-18, IL-18BP, IL-19, IL-1A, IL-1B, IL-1F10, IL-1F3/IL-1RA, IL-1F5, IL-1F6, IL-1F7, IL-1F8, IL-1F9, IL-1-like, IL-1RA, IL-1RL2, IL-1c, IL-103, IL-2, IL-20, IL-21, IL-22, IL-23, IL-24, IL-28A, IL-28B, IL-29, IL-30, IL-31, IL-32, IL-33, IL-34, IL-35, IL-36, IL-37, IL-38, IL-39, or IL-40, IL-4, IL-5, IL-6, IL-6-like, IL-7, IL-8/CXCL8, IL-9, inflammasome, interferome, interferon alpha (IFN-α), interferon beta-1a, interferon beta-1b, interferon gamma, interferon type 1, interferon type II, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MHC class III, miscellaneous hematopoietins, monokine, MSP, myokine, myonectin, nicotinamide phosphoribosyltransferase, oncostatin M (OSM), oprelvekin, OX40L, platelet factor 4, promegapoietin, RANKL, SCF, STAT3, STAT4, STAT6, stromal cell-derived factor 1, TALL-1, TBX21, TGF-α, TGF-β, TGF-β1, TGF-β2, TGF-β3, TNF, TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2. In some embodiments, cytokine encoded by the engineered polynucleotide described herein can include IL-2, IL-12, IL-15, IFN-α, or a combination thereof. In some embodiments, cytokine encoded by the engineered polynucleotide described herein is IL-2, IL-12, IL-15, and IFN-α.

In some embodiments, the at least one cytokine comprises a modified cytokine. For example, the modified cytokine comprises secreted cytokine, membrane tethered cytokine, masked cytokine, cytokine fusion, or a combination thereof. In some embodiments, the cytokine fusion comprises a cytokine coupled to an antibody or fragment thereof. In some embodiments, the cytokine fusion comprises a cytokine coupled to an Fc region of the antibody or fragment thereof. In some embodiments, the cytokine to be an engineered cytokine comprising a masking domain, a half-life extension domain, a linker, a signal peptide, or a combination thereof. In some embodiments, the masking domain is covalently connected to the engineered by a cleavable linker, where a masking domain can be cleaved off and removed from the engineered cytokine by a cancer cell specific protease, thereby limiting the activity or signaling cascade of the engineered cytokine to the cancer cell or within the proximity of the cancer cell (e.g., a cancer microenvironment). In some embodiments, the half-life extension domain comprises an antibody or fragment thereof. In some embodiments, the half-life extension domain comprises fragment crystallizable region (Fc region) or fragment thereof. In some embodiments, the half-life extension domain comprises a serum protein binding domain or fragment thereof. In some embodiments, the fusion comprises a cytokine or interferon coupled to human albumin binding domain (HSA). In some embodiments, the composition further comprises at least one additional active ingredient. For example, the at least one additional active ingredient comprises an immune checkpoint inhibitor, where the immune checkpoint inhibitor comprises one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, TACTILE (CD96), PVRIG (CD1112R), CD137, or a combination thereof. In some embodiments, the immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab.

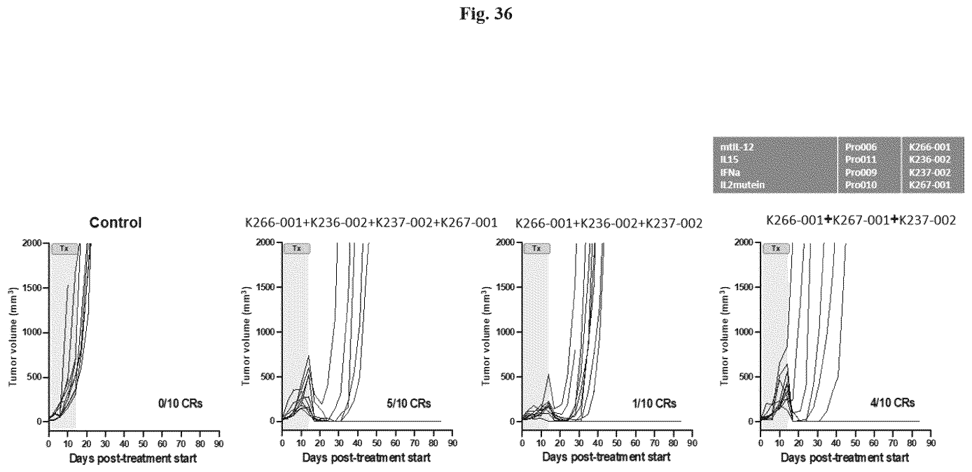

In some embodiments, the composition comprises more than one engineered polynucleotide for encoding a combination of the engineered therapeutics. In some embodiments, the combination of therapeutics comprises two, three, four, or more therapeutics. In some embodiments, the combination of therapeutics comprises two, three, four, or more interleukins, interferons, or a combination thereof. In some embodiments, the combination of therapeutics increases therapeutic efficacies for treating a disease or condition described herein compared to administration of individual therapeutics. In some embodiments, an unique combination of therapeutics increases therapeutic efficacies compared to other combinatorial therapies. As shown in FIGS. 36-50, the specific combination of an engineered therapeutic cocktail (IL-2, IL-12, IL-15, and IFN-α) increases therapeutic efficacies of treating tumors and cancer while not increasing adverse side effects. In some embodiments, the any one of the cocktail combination (e.g., IL-2, IL-12, IL-15, or IFN-α) can be substituted with an engineered cytokine described herein. For example, cocktail composition can include an engineered IL-2 or engineered IL-12 or both in place of IL-2 or IL-12. Such substitution with the engineered cytokine can further increase (e.g., synergistically) the engineered therapeutic efficacies of treatment by the combination.

In some embodiments, the engineered therapeutic is encoded from a nucleic acid sequence that is at least 75%, at least 80%, at least 85%, at least 90%, at least 95%, or at least 99% identical to any one of SEQ ID NOs: 461-476. In some embodiments, the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 351-361, 371-373, 481-490, 501-510, or 601-685. In some embodiments, the engineered therapeutic comprises an engineered cytokine comprising a secreted cytokine, a membrane tethered cytokine, a masked cytokine, a cytokine fusion, or a combination thereof. In some embodiments, the cytokine comprises an interleukin or an interferon. In some embodiments, the interleukin comprises an IL-2. In some embodiments, the IL-2 is encoded from a nucleic acid sequence that is at least 75%, at least 80%, at least 85%, at least 90%, at least 95%, or at least 99% identical to any one of SEQ ID NOs: 467 or 468. In some embodiments, the IL-2 comprises an amino acid sequence that is at least 75%, at least 80%, at least 85%, at least 90%, at least 95%, or at least 99% identical to any one of SEQ ID NOs: 351-361, 371-373, 481, 482, 501, 502, 614-621, or 664-667. In some embodiments, the interleukin comprises an IL-12. In some embodiments, the IL-12 is encoded from a nucleic acid sequence that is at least 75%, at least 80%, at least 85%, at least 90%, at least 95%, or at least 99% identical to any one of SEQ ID NOs: 461, 462, 475, or 476. In some embodiments, the IL-12 comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, 503-507, or 648-663. In some embodiments, the interleukin comprises an IL-15. In some embodiments, the IL-15 is encoded from a nucleic acid sequence that is at least 75%, at least 80%, at least 85%, at least 90%, at least 95%, or at least 99% identical to any one of SEQ ID NOs: 463, 464, 469, or 470. In some embodiments, the IL-15 comprises an amino acid sequence that is at least 75%, at least 80%, at least 85%, at least 90%, at least 95%, or at least 99% identical to SEQ ID NOs: 488 489, 508, 509, 622-647, 668, or 669. In some embodiments, the cytokine comprises the interferon. In some embodiments, the interferon comprises an IFN-α. In some embodiments, the IFN-α is encoded from a nucleic acid sequence that is at least 75%, at least 80%, at least 85%, at least 90%, at least 95%, or at least 99% identical to any one of SEQ ID NOs: 465 or 466. In some embodiments, the IFN-α comprises an amino acid sequence that is at least 75%, at least 80%, at least 85%, at least 90%, at least 95%, or at least 99% identical to SEQ ID NOs: 490, 510, 601-613, 674, 678, or 685.

### Engineered Therapeutic

Described herein, in some aspects, are engineered therapeutics encoded by the engineered polynucleotide described herein. In some embodiments, the expression of the engineered therapeutic is increased in a cancer cell compared to expression of the engineered therapeutic in a normal cell. In some embodiments, the activity of the engineered therapeutic is increased in a cancer cell compared to activity of the engineered therapeutic in a normal cell. For example, the engineered therapeutic can include a masking domain, where the masking domain inhibits or decreases enzymatic, signaling, or receptor binding activity of the engineered therapeutic. A cancer cell-specific protease can cleave the masking domain from the engineered therapeutic, thus activating the engineered therapeutic in a cancer cell as opposed to a normal cell. In some embodiments, the combination of oncoselective expression of the engineered therapeutic in a cancer cell and the activation of the engineered therapeutic in the same cancer cell synergistically increases the effectiveness of treating and killing the cancer cell.

In some embodiments, the engineered therapeutic described herein is an engineered cytokine. In some embodiments, the engineered therapeutic comprises: an engineered interleukin or fragment thereof; or an engineered subunit of an interleukin. In some embodiments, the engineered therapeutic comprises a fusion of two subunits. For example, the engineered therapeutic can be a fusion of p35 subunit and p40 subunit for forming a single chain of IL-12. In some embodiments, the engineered therapeutic comprises a masking domain. In some embodiments, the engineered therapeutic comprises a half-life extension domain. In some embodiments, the engineered therapeutic comprises a linker. In some embodiments, the linker is a non-cleavable linker. In some embodiments, the non-cleavable linker is (GGGGS)n, where n can be any integer. In some embodiments, the non-cleavable linker is (GGGGS)n, where n is three, yielding a non-cleavable linker having an amino acid sequence of GGGGSGGGGSGGGGS (SEQ ID NO: 172). In some embodiments, the linker comprises an amino acid sequence of GG(SGG)n, where n is an integer. In some embodiments, the linker comprises GG(SGG)n, where n is six for a linker having an amino acid sequence of GGSGGSGGSGGSGGSGGSGG (SEQ ID NO: 173). In some embodiments, the linker is a cleavable linker. In some embodiments, the cleavable linker comprises an amino acid sequence of HPVGLLARVPLSLYSGHPVGLLARVPLSLYSGLSGRSDNHGGGSGGGS (SEQ ID NO: 174). In some embodiments, the cleavable linker comprises an amino acid sequence of GPPSGSSPMPYDLYHPSGGG (SEQ ID NO: 175). In some embodiments, the engineered therapeutic comprises a signal peptide (e.g., a mouse Kappa light chain signal peptide. In some embodiments, the signal peptide comprises an amino acid sequence of METDTLLLWVLLLWVPGSTG (SEQ ID NO: 176). In some embodiments, the signal peptide comprises an amino acid sequence of MEFGLSWVFLVALFRGVQC ( ). In some embodiments, the signal peptide comprises an amino acid sequence of MYRMQLLSCI ALSLALVTNS (SEQ ID NO: 178).

In some embodiments, a masking domain as provided herein refers to a domain for binding or inhibiting functional domain of a cytokine. In some embodiments, the presence of the masking domain inhibits or decreases activity of the masked cytokine. For example, a masking domain for an IL-12 or an IL-2 can inhibit interaction or binding between the IL-12 or the IL-2 of binding and its respective cognate receptor or protein, thereby decreasing activity or signaling cascade associated with IL-12 or IL-2. In some embodiments, the masking domain is functionally coupled to the engineered cytokine described herein. In some embodiments, the masking domain is covalently connected to the engineered cytokine described herein. In some embodiments, the masking domain is covalently connected to the engineered cytokine by a linker. In some embodiments, the linker can be a cleavable linker. In such case, the masking domain can be cleaved off and removed from the engineered therapeutic by a cancer cell specific protease, thereby limiting the activity or signaling cascade of the engineered cytokine to the cancer cell or within the proximity of the cancer cell (e.g., a cancer microenvironment).

In some embodiments, the masking domain comprises a receptor or fragment thereof of a cytokine described herein. In some embodiments, the masking domain comprises a receptor or fragment thereof of a interleukin described herein. In some embodiments, the masking domain comprises a receptor or fragment thereof of an IL-12 or IL-2. In some embodiments, the masking domain comprises a antibody or antigen binding fragment thereof of an IL-12 or IL-2. In some embodiments, the masking domain comprises interleukin 12 receptor subunit beta 1 (IL-12RB1) or fragment thereof. In some embodiments, the masking domain comprises an amino acid sequence that is at least 75%, at least 80%, at least 85%, at least 90%, at least 95%, or at least 99% identical to QLGASGPGDGCCVEKTSFPEGASGSPLGPRNLSCYRVSKTDYECSWQYDGPEDNVS HVLWCCFVPPNHTHTGQERCRYFSSGPDRTVQFWEQDGIPVLSKVNFWVESRLGNR TMKSQKISQYLYNWTKTTPPLGHIKVSQSHRQLRMDWNVSEEAGAEVQFRRRMPTT NWTLGDCGPQVNSGSGVLGDIRGSMSESCLCPSENMAQEIQIRRRRRLSSGAPGGPWSD WSMPVCVPPEVLPQA (SEQ ID NO: 181). In some embodiments, the masking domain comprises an amino acid sequence of SEQ ID NO: 181. In some embodiments, the masking domain comprises a receptor or fragment thereof of an IL-12 or IL-2. In some embodiments, the masking domain comprises interleukin 12 receptor subunit beta 2 (IL-12RB2) or fragment thereof. In some embodiments, the masking domain comprises an amino acid sequence at least 75%, at least 80%, at least 85%, at least 90%, at least 95%, or at least 99% identical to NIDVCKLGTVTVQPAPVIPLGSAANISCSLNPKQGCSHYPSSNELILLKFVNDVLVENLH GKKVHDHTGHSSTFQVTNLSLGMTLFVCKLNCSNSQKKPPVPVCGVEISVGVAPEPPQN ISCVQEGENGTVACSWNSGKVTYLKTNYTLQLSGPNNLTCQKQCFSDNRQNCNRLDLG INLSPDLAESRFIVRVTAINDLGNSSSLPHTFTFLDIVIPLPPWDIRINFLNASGSRGTLQWE DEGQVVLNQLRYQPLNSTSWNMVNATNAKGKYDLRDLRPFTEYEFQISSKLHLSGGS WSNWSESLRTRTPEE (SEQ ID NO: 182). In some embodiments, the masking domain comprises an amino acid sequence of SEQ ID NO: 182. In some embodiments, the masking domain comprises human interleukin 12 receptor subunit beta 1 (IL-12RB1) or fragment thereof. In some embodiments, the masking domain comprises an amino acid sequence at least 75%, at least 80%, at least 85%, at least 90%, at least 95%, or at least 99% identical to CRTSECCFQDPPYPDADSGSASGPRDLRCYRISSDRYECSWQYEGPTAGVSHFLRCCLSS GRCCYFAAGSATRLQFSDQAGVSVLYTVTLWVESWARNQTEKSPEVTLQLYNSVKYE PPLGDIKVSKLAGQLRMEWETPDNQVGAEVQFRHRTPSSPWKLGDCGPQDDDTESCLC PLEMNVAQEFQLRRRQLGSQGSSWSKWSSPVCVPPENP (SEQ ID NO: 183). In some embodiments, the masking domain comprises an amino acid sequence of SEQ ID NO: 183. In some embodiments, the masking domain comprises human interleukin 12 receptor subunit beta 2 (IL-12RB2) or fragment thereof. In some embodiments, the masking domain comprises an amino acid sequence at least 75%, at least 80%, at least 85%, at least 90%, at least 95%, or at least 99% identical to KIDACKRGDVTVKPSHVILLGSTVNITCSLKPRQGCFHYSRRNKLILYKFDRRINFHHGH SLNSQVTGLPLGTTLFVCKLACINSDEIQICGAEIFVGVAPEQPQNLSCIQKGEQGTVACT WERGRDTHLYTEYTLQLSGPKNLTWQKQCKDIYCDYLDFGINLTPESPESNFTAKVTA VNSLGSSSSLPSTFTFLDIVRPLPPWDIRIKFQKASVSRCTLYWRDEGLVLLNRLRYRPSN SRLWNMVNVTKAKGRHDLLDLKPFTEYEFQISSKLHLYKGSWSDWSESLRAQTPEE (SEQ ID NO: 184). In some embodiments, the masking domain comprises an amino acid sequence of SEQ ID NO: 184. In some embodiments, the masking domain comprises human interleukin 2 receptor subunit beta 2 (IL-2Rbeta) or fragment thereof. In some embodiments, the masking domain comprises an amino acid sequence at least 75%, at least 80%, at least 85%, at least 90%, at least 95%, or at least 99% identical to AVNGTSQFTCFYNSRANISCVWSQDGALQDTSCQVHAWPDRRRWNQTCELLPVSQAS WACNLILGAPDSQKLTTVDIVTLRVLCREGVRWRVMAIQDFKPFENLRLMAPISLQVV HVETHRCNISWEISQASHYFERHLEFEARTLSPGHTW EEAPLLTLKQKQEWICLETLTPD TQYEFQVRVKPLQ (SEQ ID NO: 185). In some embodiments, the masking domain comprises an amino acid sequence of SEQ ID NO: 185. In some embodiments, the masking domain comprises a single chain Fab (scFab) of IL-2. In some embodiments, the masking domain comprises an amino acid sequence at least 75%, at least 80%, at least 85%, at least 90%, at least 95%, or at least 99% identical to EVQLVESGGGLVQPGGSLRLSCAASGFTFSSYTLAWVRQAPGKGLEWVAAIDSSSYTY SPDTVRGRFTISRDNAKNSLYLQMNSLRAEDTAVYYCARDSNWDALDYWGQGTTVTV SSGGGGSGGGGSGGGGSDIQMTQSPSSLSASVGDRVTITCKASQNVGTNVGWYQQKPG KAPKALIYSASFRYSGVPSRFSGSGSGTDFTLTISSLQPEDFATYYCQQYYTYPYTFGGG TKVEIK (SEQ ID NO: 186). In some embodiments, the masking domain comprises an amino acid sequence of SEQ ID NO: 186.

In some embodiments, the engineered therapeutic comprises a half-life extension domain. In some embodiments, the half-life extension domain comprises an antibody or fragment thereof. In some embodiments, the antibody or fragment thereof includes variants, derivatives, and antigen-binding fragments thereof. In some embodiments, an antibody can be a single domain antibody (sdAb), for example, a heavy chain only antibody (HCAb) VHH, or nanobody. Non-limiting examples of antigen-binding fragments include Fab, Fab′, F(ab′)2, dimers and trimers of Fab conjugates, Fv, scFv, minibodies, dia-, tria-, and tetrabodies, and linear antibodies. Fab and Fab′ are antigen-binding fragments that can comprise the VH and CH1 domains of the heavy chain linked to the VL and CL domains of the light chain via a disulfide bond. A F(ab′)2 can comprise two Fab or Fab′ that are joined by disulfide bonds. A Fv can comprise the VH and VL domains held together by non-covalent interactions. A scFv (single-chain variable fragment) is a fusion protein that can comprise the VH and VL domains connected by a peptide linker. Manipulation of the orientation of the VH and VL domains and the linker length can be used to create different forms of molecules that can be monomeric, dimeric (diabody), trimeric (triabody), or tetrameric (tetrabody). Minibodies are scFv-CH3fusion proteins that assemble into bivalent dimers. In some embodiments, the antibody or fragment thereof includes those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity).

In some embodiments, the half-life extension domain comprises fragment crystallizable region (Fc region) or fragment thereof. In some embodiments, the half-life extension domain comprises an amino acid sequence that is at least 75%, at least 80%, at least 85%, at least 90%, at least 95%, or at least 99% identical to GPTIKPCPPCKCPAPNAAGGPSVFIFPPKIKDVLMISLSPIVTCVVVDVSEDDPDVQISWF VNNVEVHTAQTQTHREDYNSTLRVVSALPIQHQDWMSGKEFKCKVNNKDLGAPIERTI SKPKGSVRAPQVYVLPPPEEEMTKKQVTLTCMVTDFMPEDIYVEWTNNGKTELNYKN TEPVLDSDGSYFMYSKLRVEKKNWVERNSYSCSVVHEGLHNHHTTKSFSRTPG (SEQ ID NO: 191). In some embodiments, the half-life extension domain comprises an amino acid sequence of SEQ ID NO: 191. In some embodiments, the half-life extension domain comprises an amino acid sequence that is at least 75%, at least 80%, at least 85%, at least 90%, at least 95%, or at least 99% identical to EPKSCDKTHTCPPCPAPEAAGGPSVFLFPPKPKDTLMISRTPEVTCVVVDVSHEDPEVKF NWYVDGVEVHNAKTKPREEQYNSTYRVVSVLTVLHQDWLNGKEYKCKVSNKALGAP IEKTISKAKGQPREPQVYTLPPSRDELTKNQVSLTCLVKGFYPSDIAVEWESNGQPENNY KTTPPVLDSDGSFFLYSKLTVDKSRWQQGNVFSCSVMHEALHNHYTQKSLSLSPGK (SEQ ID NO: 192). In some embodiments, the half-life extension domain comprises an amino acid sequence of SEQ ID NO: 192. In some embodiments, the half-life extension domain comprises an amino acid sequence that is at least 75%, at least 80%, at least 85%, at least 90%, at least 95%, or at least 99% identical to PRGPTIKPCPPCKCPAPNAAGGPSVFIFPPKIKDVLMISLSPIVTCVVVDVSEDDPDVQIS WFVNNVEVHTAQTQTHREDYNSTLRVVSALPIQHQDWMSGKEFKCKVNNKDLGAPIE RTISKPKGSVRAPQVYVLPPPEEEMTKKQVTLTCMVTDFMPEDIYVEWTNNGKTELNY KNTEPVLdSDGSYfMYSKLRVEKKNWVERNSYSCSVVHEGLHNHHTTKSFSRTPGK (SEQ ID NO: 193). In some embodiments, the half-life extension domain comprises an amino acid sequence of SEQ ID NO: 193.

In some embodiments, the half-life extension domain comprises a serum protein binding domain or fragment thereof. In some embodiments, the half-life extension domain comprises a human serum binding domain. In some embodiments, the half-life extension domain comprises an amino acid sequence that is at least 75%, at least 80%, at least 85%, at least 90%, at least 95%, or at least 99% identical to EVQLVESGGGLVQPGNSLRLSCAASGFTFSKFGMSWVRQAPGKGLEWVSSISGSGRDT LYAESVKGRFTISRDNAKTTLYLQMNSLRPEDT AVYYCTIGGSLSVSSQGTLVTVSS (SEQ ID NO: 194). In some embodiments, the half-life extension domain comprises an amino acid sequence of SEQ ID NO: 194.

In some embodiments, the engineered therapeutic comprises an engineered interleukin or fragment thereof; or an engineered subunit of an interleukin. In some embodiments, the engineered interleukin or fragment thereof or the engineered subunit of the interleukin comprises IL-12 or subunit of IL-12. In some embodiments, the engineered IL-12 comprises a p40 subunit or an engineered p40 subunit. In some embodiments, the p40 subunit comprises an amino acid sequence at least 75%, at least 80%, at least 85%, at least 90%, at least 95%, or at least 99% identical to MWELEKDVYVVEVDWTPDAPGETVNLTCDTPEEDDITWTSDQRHGVIGSGKTLTITVK EFLDAGQYTCHKGGETLSHSHLLLHKKENGIWSTEILKNFKNKTFLKCEAPNYSGRFTC SWLVQRNMDLKFNIKSSSSSPDSRAVTCGMASLSAEKVTLDQRDYEKYSVSCQEDVTC PTAEETLPIELALEARQQNKYENYSTSFFIRDIIKPDPPKNLQMKPLKNSQVEVSWEYPDS WSTPHSYFSLKFFVRIQRKKEKMKETEEGCNQKGAFLVEKTSTEVQCKGGNVCVQAQD RYYNSSCSKWACVPCRVRS (SEQ ID NO: 201). In some embodiments, the p40 subunit comprises an amino acid sequence of SEQ ID NO: 201. In some embodiments, the engineered IL-12 comprises a p35 subunit or an engineered p35 subunit. In some embodiments, the p35 subunit comprises an amino acid sequence at least 75%, at least 80%, at least 85%, at least 90%, at least 95%, or at least 99% identical to RVIPVSGPARCLSQSRNLLKTTDDMVKTAREKLKHYSCTAEDIDHEDITRDQTSTLKT CLPLELHKNESCLATRETSSTTRGSCLPPQKTSLMMTLCLGSIYEDLKMYQTEFQAIN AALQNHNHQQIILDKGMLVAIDELMQSLNHNGETLRQKPPVGEADPYRVKMKLCIL LHAFSTRVVTINRVMGYLSSA (SEQ ID NO: 202). In some embodiments, the p40 subunit comprises an amino acid sequence of SEQ ID NO: 202.

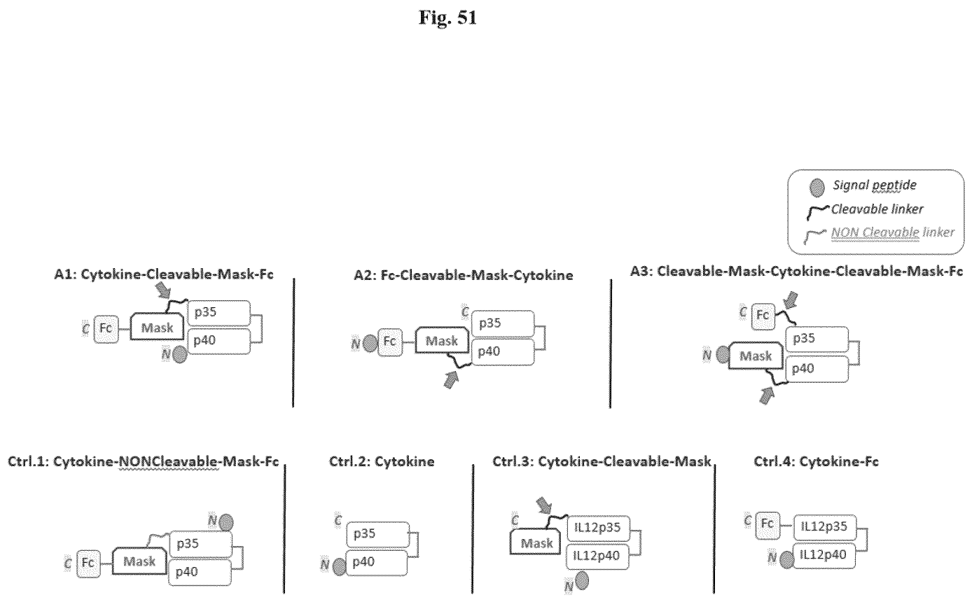

In some embodiments, the engineered IL-12 comprises a fusion of two IL-12 subunits. For example, the engineered IL-12 can include a p40-p40 fusion, a p40-p35 fusion, or a p30-p30 fusion. In some embodiments, the engineered IL-12 fusion comprises a masking domain, a linker, a signal peptide, a half-life extension domain, or a combination thereof as illustrated in FIG. 51. In some embodiments, the engineered IL-12 comprising a fusion of two IL-12 subunits comprises an amino acid sequence that is at least 75%, at least 80%, at least 85%, at least 90%, at least 95%, or at least 99% identical to any one of SEQ ID NOs: 301-303. In some embodiments, the engineered IL-12 comprises an amino acid sequence that is at any one of SEQ ID NOs: 301-303. In some embodiments, the engineered IL-12 comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 301-303.

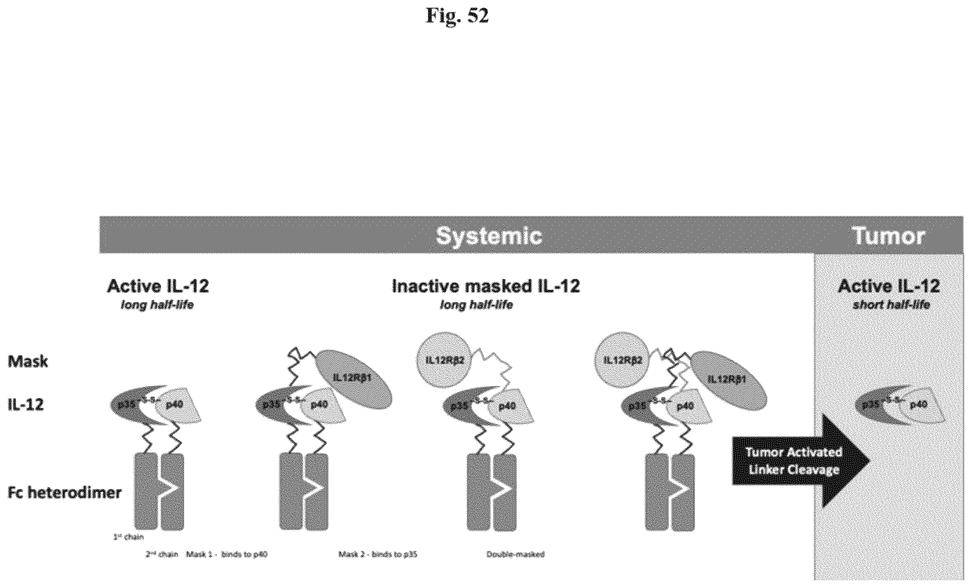

In some embodiments, the engineered IL-12 comprises an engineered subunits of IL-12, a masking domain, a linker, a signal peptide, a half-life extension domain, or a combination thereof as illustrated in FIG. 52. In some embodiments, the engineered IL-12 comprising the engineered subunit of IL-12 comprises an amino acid sequence that is at least 75%, at least 80%, at least 85%, at least 90%, at least 95%, or at least 99% identical to any one of SEQ ID NOs: 311-314 or 321-328. In some embodiments, the engineered IL-12 comprises an amino acid sequence that is any one of SEQ ID NOs: 311-314 or 321-328. In some embodiments, the engineered IL-12 comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 311-314 or 321-328.

In some embodiments, the engineered IL-12 comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, or 648-663. In some embodiments, the engineered IL-12 comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 301-303, 311-314, 321-328, 331-335, 341-344, 483-487, or 648-663.

In some embodiments, the engineered therapeutic comprises an engineered IL-2. In some embodiments, the engineered IL-2 comprises a masking domain, a linker, a signal peptide, a half-life extension domain, or a combination thereof. In some embodiments, the engineered IL-2 is a secreted IL-2 (e.g., mediated by a secretory signal peptide comprising SEQ ID NO: 178). In some embodiments, the engineered IL-2 comprises an amino acid sequence that is at least 75%, at least 80%, at least 85%, at least 90%, at least 95%, or at least 99% identical to APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 211). In some embodiments, the engineered IL-2 comprises an amino acid sequence that is at least 75%, at least 80%, at least 85%, at least 90%, at least 95%, or at least 99% identical to any one of SEQ ID NOs: 351-361, 371-373, 481, 482, 614-621, or 664-667. In some embodiments, the engineered IL-2 comprises an amino acid sequence that is at any one of SEQ ID NOs: 351-361, 371-373, 481, 482, 614-621, or 664-667. In some embodiments, the engineered IL-2 comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 351-361, 371-373, 481, 482, 614-621, or 664-667.

In some embodiments, the engineered therapeutic described herein comprises an engineered IL-15. In some embodiments, the engineered IL-15 comprises an IL-15 fusion. In some embodiments, the fusion comprises a sushi domain. In some embodiments, the engineered IL-15 comprises an IL-15 fused to a sushi domain. In some embodiments, the engineered therapeutic comprising the IL-15 comprises an amino acid sequence that is at least 75%, at least 80%, at least 85%, at least 90%, at least 95%, or at least 99% identical to SEQ ID NO: 488 489, 622-647, 668, or 669. In some embodiments, the engineered therapeutic comprising the IL-15 comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to SEQ ID NO: 488 489, 622-647, 668, or 669.

In some embodiments, the engineered therapeutic comprises an interferon. In some embodiments, the interferon comprises an interferon alpha (IFN-α). In some embodiments, the engineered therapeutic comprising the IFN-α comprises an amino acid sequence that is at least 75%, at least 80%, at least 85%, at least 90%, at least 95%, or at least 99% identical to SEQ ID NOs: 490, 601-613, 674, 678, or 685. In some embodiments, the engineered therapeutic comprising the IFN-α comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to SEQ ID NOs: 490, 601-613, 674, 678, or 685.

In some embodiments, the engineered therapeutic is encoded by the engineered polynucleotide comprising a nucleic acid sequence that is at least 75%, at least 80%, at least 85%, at least 90%, at least 95%, or at least 99% identical to any one of SEQ ID NOs: 461-476. In some embodiments, the engineered therapeutic is encoded by the engineered polynucleotide comprising a nucleic acid sequence that is any one of SEQ ID NOs: 461-476. In some embodiments, the engineered therapeutic is encoded by the engineered polynucleotide comprising a nucleic acid sequence that is at least 100 contiguous nucleotides, at least 150 contiguous nucleotides, at least 200 contiguous nucleotides, at least 250 contiguous nucleotides, at least 300 contiguous nucleotides, at least 350 contiguous nucleotides, at least 400 contiguous nucleotides, at least 450 contiguous nucleotides, at least 500 contiguous nucleotides, at least 550 contiguous nucleotides, or at least 600 contiguous nucleotides to any one of SEQ ID NOs: 261-276. In some embodiments, the engineered therapeutic comprising IL-2 comprises SEQ ID NOs: 467 or 468. In some embodiments, the engineered therapeutic comprising IL-12 comprises SEQ ID NOs: 461, 462, 475, 476, or 503-507. In some embodiments, the engineered therapeutic comprising IL-15 comprises SEQ ID NOs: 463, 464, 469, 470, 508, or 509. In some embodiments, the engineered therapeutic comprising IFN-α comprises SEQ ID NOs: 465, 466, or 510.

In some embodiments, the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 351-361, 371-373, 481-490, 501-510, or 601-685. In some embodiments, the engineered therapeutic comprises an amino acid sequence that is at any one of SEQ ID NOs: 301-303, 311-314, 321-328, 331-335, 341-344, 351-361, 371-373, 481-490, 501-510, or 601-685. In some embodiments, the engineered therapeutics comprises an amino acid sequence that is at least 50 contiguous amino acids, at least 100 contiguous amino acids, or at least 150 contiguous amino acids identical to any one of SEQ ID NOs: 301-303, 311-314, 321-328, 331-335, 341-344, 351-361, 371-373, 481-490, 501-510, or 601-685.

### Lipid

In some embodiments, the composition or the pharmaceutical composition described herein comprises at least one lipid or lipid derivative thereof. In some embodiments, an engineered polynucleotide described herein can be encapsulated or complexed with the at least one lipid or lipid derivative. For example, the composition or the pharmaceutical composition can comprise a liposome, a lipioid, a lipid nanoparticle, or a combination thereof. In some embodiments, the synthetic polynucleotide or the vector described herein can be functionally coupled (e.g., crosslinked) to the lipid or the lipid derivative. Non-limiting example of a liposome can include 1, 2-dioleyloxy-N, N-dimethylaminopropane (DODMA) liposome, DiLa2 liposome, 1, 2-dilinoleyloxy-3-dimethylaminopropane (DLin-DMA), 2, 2-dilinoleyl-4-(2-dimethylaminoethyl)-[1,3]-dioxolane (DLin-KC2-DMA), or MC3. In some embodiments, the liposome comprises different sizes such as a multilamellar vesicle, a small unicellular vesicle, or a large unilamellar vesicle. In some embodiments, the liposome comprises a neutral lipid such as cholesterol or dioleoyl phosphatidylethanolamine (DOPE). In some embodiments, the liposome comprises a cationic lipid such as, DLin-MC3-DMA, DLin-DMA, C12-200, or DLin-KC2-DMA. In some embodiments, the composition or the pharmaceutical composition comprises a lipid nanoparticle (LNP). Non-limiting example of LNP can include a combination of PEG-DMG 2000, DSPC, or cholesterol.

### Pharmaceutical Composition

Described herein are pharmaceutical compositions comprising the engineered polynucleotide described herein. In some embodiments, the pharmaceutical composition further comprises a pharmaceutically acceptable: carrier, excipient, or diluent. In some embodiments, the pharmaceutical composition comprises two or more engineered therapeutics or therapeutics as disclosed herein. In some embodiments, the pharmaceutical composition comprises at least one additional active ingredient as disclosed herein. In some embodiments, the pharmaceutical composition comprising the engineered polynucleotide described herein treats a disease or condition described herein. In some embodiments, the disease or condition comprises a cancer. In some embodiments, the cancer comprises Acute Lymphoblastic Leukemia, Acute Lymphocytic Leukemia (ALL), Acute Myeloid Leukemia (AML), Adenoid Cystic Carcinoma, Adrenal Gland Cancer, Adrenocortical Carcinoma, Adult Leukemia, AIDS-Related Lymphoma, Amyloidosis, Anal Cancer, Astrocytomas, Ataxia Telangiectasia, Atypical Mole Syndrome, Atypical Teratoid/Rhabdoid Tumor, Basal Cell Carcinoma, Bile Duct Cancer, Birt Hogg Dube Syndrome, Bladder Cancer, Bone Cancer, Brain Tumor, Breast Cancer, Bronchial Tumors, Burkitt Lymphoma, Carcinoid Tumor (Gastrointestinal), Carcinoma of Unknown Primary, Cardiac (Heart) Tumors, Cervical Cancer, Cholangiocarcinoma, Chordoma, Chronic Lymphocytic Leukemia (CLL), Chronic Myelogenous Leukemia, Chronic Myeloid Leukemia, Chronic Myeloproliferative Neoplasms, Colorectal Cancer, Craniopharyngioma, Cutaneous T-Cell Lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Malignant, and Osteosarcoma, Gallbladder Cancer, Gastric Cancer, Gastrointestinal Carcinoid Tumor, Gastrontestinal Stromal Tumor (GIST), Germ Cell Tumors, Gestational Trophoblastic Disease, Hairy Cell Leukemia, Head and Neck Cancer, Hepatocellular Cancer, HER2-Positive Breast Cancer, Histiocytosis, Langerhans Cell, Hodgkin's Lymphoma, Hypopharyngeal Cancer, Intraocular Melanoma, Islet Cell Tumor, Juvenile Polyposis Syndrome, Kaposi Sarcoma, Kidney Cancer, Langerhans Cell Histiocytosis, Laryngeal Cancer, Leukemia, Lip and Oral Cavity Cancer, Liver Cancer, Lobular Carcinoma, Lung Cancer (Non-Small Cell and Small Cell), Lymphoma, Malignant Fibrous Histiocytoma of Bone and Osteosarcoma, Malignant Glioma, Melanoma, Intraocular Melanoma, Meningioma, Merkel Cell Carcinoma, Mesothelioma, Malignant, Metastatic Cancer, Metastatic Squamous Neck Cancer with Occult Primary, Midline Tract Carcinoma, Multiple Endocrine Neoplasia Syndromes, Multiple Myeloma, Plasma Cell Neoplasms, Mycosis Fungoides, Myelodysplastic Syndrome (MDS), Myeloproliferative Neoplasms, Chronic, Nasal Cavity and Paranasal Sinus Cancer, Nasopharyngeal Cancer, Neuroblastoma, Neuroendocrine Tumor, Non-Hodgkin Lymphoma, Oral Cancer, Lip and Oral Cavity Cancer and Oropharyngeal Cancer, Oropharyngeal Cancer, Osteosarcoma, Ovarian Cancer, Ovarian Germ Cell Tumors, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Peritoneal Cancer, Peutz-Jeghers Syndrome, Pharyngeal Cancer, Pheochromocytoma, Pituitary Tumor, Plasma Cell Neoplasm/Multiple Myeloma, Pleuropulmonary Blastoma, Polycythemia Vera, Pregnancy and Breast Cancer, Primary Central Nervous System (CNS) Lymphoma, Primary Peritoneal Cancer, Prostate Cancer, Rectal Cancer, Recurrent Cancer, Renal Cell Carcinoma, Retinoblastoma, Rhabdomyosarcoma, Salivary Gland Cancer, Sarcoma, S6zary Syndrome, Skin Cancer, Small Intestine Cancer, Soft Tissue Sarcoma, Solid tumor, Squamous Cell Carcinoma of the Skin, Squamous Neck Cancer with Occult Primary, Metastatic, Stomach Cancer, T-Cell Lymphoma, Testicular Cancer, Throat Cancer, Thymoma, Thymic Carcinoma, Thyroid Cancer, Transitional Cell Cancer of the Renal Pelvis and Ureter, Unusual Cancers of Childhood, Ureter and Renal Pelvis, Transitional Cell Cancer, Urethral Cancer, Uterine (Endometrial) Cancer, Uterine Sarcoma, Vaginal Cancer, Vascular Tumors, Vulvar Cancer, Wilms Tumor, or a combination thereof.

In some embodiments, the cancer type is a solid cancer type or a hematologic malignant cancer type. In some embodiments, the cancer type is a metastatic cancer type or a relapsed or refractory cancer type. In some embodiments, the cancer type comprises acute myeloid leukemia (LAML or AML), acute lymphoblastic leukemia (ALL), adrenocortical carcinoma (ACC), bladder urothelial cancer (BLCA), brain stem glioma, brain lower grade glioma (LGG), brain tumor, breast cancer (BRCA), bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma (CESC) cancer, childhood cancers, cholangiocarcinoma (CHOL), chordoma, chronic lymphocytic leukemia, chronic myelogenous leukemia, chronic myeloproliferative disorders, colon (adenocarcinoma) cancer (COAD), colorectal cancer, craniopharyngioma, cutaneous T-cell lymphoma, endocrine pancreas islet cell tumors, endometrial cancer, ependymoblastoma, ependymoma, esophageal cancer (ESCA), esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioblastoma multiforme glioma GBM), hairy cell leukemia, head and neck cancer (HNSD), heart cancer, Hodgkin lymphoma, hypopharyngeal cancer, intraocular melanoma, islet cell tumors, Kaposi sarcoma, kidney cancer, Langerhans cell histiocytosis, laryngeal cancer, lip cancer, liver cancer, Lymphoid Neoplasm Diffuse Large B-cell Lymphoma (DLBCL), malignant fibrous histiocytoma bone cancer, medulloblastoma, medullo epithelioma, melanoma, Merkel cell carcinoma, Merkel cell skin carcinoma, mesothelioma (MESO), metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma (PCPG), pineal parenchymal tumors of intermediate differentiation, pineoblastoma, pituitary tumor, plasma cell neoplasm/multiple myeloma, pleuropulmonary blastoma, primary central nervous system (CNS) lymphoma, primary hepatocellular liver cancer, prostate cancer such as prostate adenocarcinoma (PRAD), rectal cancer, renal cancer, renal cell (kidney) cancer, renal cell cancer, respiratory tract cancer, retinoblastoma, rhabdomyosarcoma, salivary gland cancer, sarcoma (SARC), Sezary syndrome, skin cutaneous melanoma (SKCM), small cell lung cancer, small intestine cancer, soft tissue sarcoma, squamous cell carcinoma, squamous neck cancer, stomach (gastric) cancer, supratentorial primitive neuroectodermal tumors, T-cell lymphoma, testicular cancer testicular germ cell tumors (TGCT), throat cancer, thymic carcinoma, thymoma (THYM), thyroid cancer (THCA), transitional cell cancer, transitional cell cancer of the renal pelvis and ureter, trophoblastic tumor, ureter cancer, urethral cancer, uterine cancer, uterine cancer, uveal melanoma (UVM), vaginal cancer, vulvar cancer, Waldenstrom macroglobulinemia, or Wilm's tumor.

In some embodiments, the cancer type comprises acute lymphoblastic leukemia, acute myeloid leukemia, bladder cancer, breast cancer, brain cancer, cervical cancer, cholangiocarcinoma, colon cancer, colorectal cancer, endometrial cancer, esophageal cancer, gastrointestinal cancer, glioma, glioblastoma, head and neck cancer, kidney cancer, liver cancer, lung cancer, lymphoid neoplasia, melanoma, a myeloid neoplasia, ovarian cancer, pancreatic cancer, pheochromocytoma and paraganglioma, prostate cancer, rectal cancer, squamous cell carcinoma, testicular cancer, stomach cancer, or thyroid cancer.

In some embodiments, the disease or condition comprises a cancer. In some embodiments, the cancer type is a solid cancer type or a hematologic malignant cancer type. In some embodiments, the cancer type is a metastatic cancer type or a relapsed or refractory cancer type.

For in vivo delivery, the engineered polynucleotide can be formulated into pharmaceutical compositions and can generally be administered intravitreally or parenterally (e.g., administered via an intramuscular, subcutaneous, intratumoral, transdermal, intrathecal, etc., route of administration). In some embodiments, the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

Administrations can be repeated for any amount of time. In some aspects, administering is performed: twice daily, every other day, twice a week, bimonthly, trimonthly, once a month, every other month, semiannually, annually, or biannually.

Dosage treatment may be a single dose schedule or a multiple dose schedule. Moreover, the subject may be administered as many doses as appropriate. One of skill in the art can readily determine an appropriate number of doses. In some aspects, a pharmaceutical composition is administered via intravitreal injection, subretinal injection, microinjection, or supraocular injection.

In practicing the methods of treatment or use provided herein, therapeutically effective amounts of the pharmaceutical composition described herein are administered to a mammal having a disease, disorder, or condition to be treated, e.g., cancer. In some embodiments, the mammal is a human. An engineered therapeutically effective amount may vary widely depending on the severity of the disease, the age and relative health of the subject, the potency of the engineered therapeutic agent used and other factors. The engineered therapeutic agents, and in some cases, compositions described herein, may be used singly or in combination with one or more therapeutic agents as components of mixtures.

The pharmaceutical composition described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical composition may be manufactured in a conventional manner, such as, by way of example only, by means of conventional mixing, dissolving, granulating, levigating, emulsifying, encapsulating, entrapping or compression processes.

In certain embodiments, the pharmaceutical composition provided herein includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, the pharmaceutical composition described herein is formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, an engineered therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

### Oncoselective Sequence

The present disclosure provides methods and compositions comprising one or more engineered polynucleotide(s). In some embodiments an engineered polynucleotide comprises a nucleotide sequence that includes a sequence element that is or is a complement of an oncoselective translation sequence element.

The present disclosure appreciates that studies have increasingly revealed alterations in ribosome structure and function that are associated with tumor development and/or progression. See, for example, Bastide and David Oncogenesis 2018 Apr. 7(4):34. Oncogenic ribosomes have a drastically altered translational landscape (“translatome”). In addition to more effectively translating various oncogenes, cancer ribosomes have been shown to be characterized by low translation fidelity and/or altered or increased stop codon read-through.

A variety of mechanisms have been described that may contribute to the altered function of oncogenic ribosomes. These include alterations in ribosome biogenesis, mutations in ribosomal protein genes, alterations in expression of ribosomal proteins, alterations in expression of rRNA, and/or alterations in the modification of rRNA. See, for example, Bastide and David Oncogenesis. 2018 April; 7(4): 34. Alterations in rRNA 2′-O-methylation patterns are also involved in cancer evolution. In some cancers, p53 inactivation triggers FBL overexpression and subsequent changes in rRNA methylation landscape (Marcel et al. Cancer Cell. 2013; 24:318-330). Such p53 inactivation (and/or FBL overexpression and/or changes in rRNA methylation) result(s) in impaired translational fidelity and increased translation of IRES-containing mRNAs. The gene encoding p53 protein, TP53 is the most commonly mutated tumor suppressor gene, Along with rRNA modifications, it is also closely connected with ribosome regulation through changes in ribosomal proteins. Ribosomal protein gene haploinsufficiency is found in about 43% of all cancers (Ajore et al., EMBO Mol Med. 2017; 9(4):498-507). In healthy cells, loss of both copies of any essential ribosomal protein gene is lethal. However, when a single copy of a ribosomal protein gene is lost, the stoichiometry of ribosomal proteins is altered and ribosomal proteins RPL5 and RPL11 have higher free (unbound) forms, which together with 5S rRNA, bind to MDM2 and stabilize p53 to stimulate growth arrest or apoptosis. This p53 mediated control mechanism in healthy cells is termed “impaired ribosome biogenesis checkpoint (Gentilella et al. Mol Cell. 2017; 67(1):55-70.e4).” In addition to TP53, retinoblastoma (RB1) gene, another commonly mutated tumor suppressor gene, is also involved in ribosome regulation, suppressing translational read-through in MYC oncogene-transformed senescent human cells (del Toro et al. BioRxiv. 2019; 10.1101/788380).

The present disclosure appreciates that oncoselective read-through can be harnessed as a powerful strategy for treatment of cancer. The present disclosure builds upon extensive work in the field of nucleic acid therapeutics (and particularly including RNA, such as mRNA engineered therapeutics), among other things by providing technologies that ensure expression of a payload included in and/or encoded by such a nucleic acid is selectively or specifically expressed in tumor cells (relative to non-tumor cells).

By providing true oncoselective or oncospecific expression, the present disclosure reduces or obviates a need to develop and/or utilize targeted (e.g., oncoselective) delivery strategies that may be required in contexts where oncoselective or oncospecific payload expression cannot be achieved. Of course, those skilled in the art, reading the present disclosure, will appreciate that any available such oncoselective delivery technology may, in some embodiments, be desirably combined with provided technologies; it is simply not required.

Alternatively or additionally, by providing true oncoselective or oncospecific expression, the present disclosure creates an option to utilize payloads that might be inappropriate or undesirable without such a high degree of selectivity. For example, as discussed herein, cytotoxic payloads (e.g., such as toxins, and pro-necroptotic, pro-pyroptotic, and pro-apoptotic proteins) might have unacceptable side effect and/or toxicology profiles when utilized with technologies that cannot ensure oncoselectivity to the extent described herein.

Among other things, the present disclosure encompasses the recognition that different ribosomes (e.g., ribosomes in tumor cells—e.g., oncogenic ribosomes-vs ribosomes in non-tumor cells—e.g., non-oncogenic ribosomes) have different processivity and/or read-through properties (e.g., different responses to pause structures and/or stop codons that impact processivity therethrough). In some embodiments, oncogenic ribosomes have frame shifts relative to non-oncogenic ribosomes. In some embodiments, frame shifts by oncogenic ribosomes can result in expression of payload sequences described herein.

In some embodiments, oncogenic ribosomes read-through, or process through, a canonical stop codon. In some embodiments, read-through of a stop codon by an oncogenic ribosome results in translation of a stop codon into an amino acid incorporated into a nascent polypeptide. In some embodiments, read-through of a stop codon by an oncogenic ribosome results in translation of some portion or all of the downstream (3′UTR) sequences following that stop codon.

Without wishing to be bound by any particular theory, the present disclosure observes that ribosome read-through of stop codons can be caused by interactions between the 18s rRNA and an RNA (e.g., an mRNA) bound by the ribosome. For example, helices of the rRNA may interact with mRNA sequences. See Namy et al. EMBO Rep. 2001 Sep. 15; 2(9): 787-793 describing interactions of helix 17 of rRNA in S. cerevisiae with mRNA bound by the ribosome that leads to stop codon read-through. The present disclosure recognizes, among other things, that human rRNA helix 37 can interact with sequences of mRNA that contribute to stop codon read-through.

Alternatively or additionally, oncoselective ribosome stop codon read-through can be induced and/or enhanced by including one or more particular structural features in a translatable nucleic acid (e.g., an RNA such as an mRNA). In some embodiments, one or more primary structure features of a translatable nucleic acid (e.g., an RNA such as an mRNA) can be used to induce and/or enhance oncoselective stop codon read-through. Alternatively or additionally, in some embodiments, one or more secondary and/or tertiary structure features (e.g. stem loop, bulge loop, kissing loop, pseudoknots, or branch loop) of a translatable nucleic acid (e.g., an RNA such as an mRNA) can be used to induce and/or enhance oncoselective stop codon read-through. In some embodiments, a structural feature capable of inducing and/or enhancing stop codon read-through is within the first 50, 60, 70, 80, 90, 100, 110, or 120 nucleotides of the downstream flanking sequence. In some further embodiments, portions of a structural feature capable of inducing and/or enhancing stop codon read-through is comprised by the first 16 nucleotides of the downstream flanking sequence.

In some embodiments, a structural feature capable of inducing and/or enhancing stop codon read-through comprises 10, 20, 30, 40, 50 or more base paired nucleotides within the first 10, 20, 30, 40, 50, 60 or more nucleotides of the downstream flanking sequence.

In accordance with some embodiments of the present disclosure, stop codon read-through can be induced and/or enhanced through use of oncoselective read-through motifs as described herein.

Alternatively or additionally, in some embodiments, inclusion of one or more regions of high G-C content can be used to induce oncospecific stop codon read-through. For example, in some embodiments, high G-C content in the 3′UTR of a translatable nucleic acid (e.g., of an RNA such as an mRNA) can be used to induce and/or enhance oncospecific stop codon readthrough. In some embodiments, high G-C content in the nucleotides preceding a stop codon can be used to induce and/or enhance oncospecific stop codon read-through of that stop codon. In some embodiments, high G-C content in the 60 nucleotides preceding a stop codon can be used to induce and/or enhance oncospecific stop codon read-through of that stop codon. In some embodiments, high G-C content in 50 nucleotides following a stop codon can be used to induce and/or enhance oncospecific stop codon readthrough of that stop codon. In some embodiments, high G-C content in the first 120 nucleotides after a stop codon (i.e., in the 3′UTR) can be used to induce and/or enhance oncospecific stop codon readthrough of that stop codon. In some embodiments, high G-C content means a log-odds of binomial probability of 4 or greater relative to a non-readthrough transcript. In some embodiments, a readthrough motif comprises G-C content of more than 42%, more than 48%, preferably more than 54% in the downstream flanking sequence.

In some embodiments, the oncoselective readthrough motif comprises a sequence selected from the group comprising: VNNNNNNMNNMWK, NNNVWNNKGHHNH, DVHVNNNCWNNNB, MWBNNNNNNNNNN, WGNNSNHNHDNNN, VNNNNNNMNNMWK or VMNNWNKNNNNNN, wherein V stands for A, C or G, M stands for A or C, W stands for A or T/U, K stands for G or T/U, H stands for A, C or T/U, D stands for A, G or T/U, B stands for C, G or T/U, S stands for G or C, N stands for any nucleotide, within the region that spans the readthrough stop codon and the first 14 nucleotides of the downstream flanking sequence; the oncoselective readthrough motif comprises a stem loop; a bulge loop, a pseudoknot, or a combination thereof within the first 50 nucleotides of the downstream flanking sequence and part of this stem loop located preferably within stop codon and the first 16 nucleotides of the downstream flanking sequence, or a combination thereof; a stem loop comprises more than 20 base paired nucleotides within first 50 nucleotides of the downstream flanking sequence; an oncoselective readthrough motif comprises a downstream flanking sequence with a G-C content of more than 42%, more than 48%, preferably more than 54%; an oncoselective readthrough motif comprises a codon that encodes proline residue, or a combination thereof.

The present disclosure further provides an insight that inclusion of a codon resulting in introduction of proline to the nascent polypeptide can induce kinking of the nascent polypeptide, and that such kinking can be used to induce and/or enhance oncoselective stop codon read-through. Thus, in some embodiments, oncoselective stop-codon readthrough can be induced and/or enhanced by inclusion of one or more proline-encoding codons in a translatable nucleic acid, as an alternative to or in addition to one or more of the other strategies described herein for inducing and/or enhancing oncoselective stop codon read-through.

In some embodiments, a stem loop in the mRNA can induce and/or enhance stop codon read-through. In some embodiments, a stem loop inducing and/or enhancing stop codon readthrough is within approximately 20, 40, 60, 80 or 120 nucleotides of the stop codon. In some embodiments, a stem loop inducing and/or enhancing stop codon read-through is in the coding sequence just prior to the stop codon. In some embodiments, a stem loop inducing and/or enhancing stop codon read-through is in the 3′UTR. In some embodiments, a stem loop inducing and/or enhancing stop codon read-through is in the region spanning the coding region and 3′UTR boundary. In some embodiments, a bulge loop or a pseudoknot in the mRNA can induce and/or enhance stop codon read-through. In some embodiments, nucleic acid structures inducing and/or enhancing stop codon read-through have a low Gibbs free energy relative to nucleic acid structures that do not result in read-through. In some embodiments, the first 25, 50, or 75 nucleotides of the 3′UTR of a nucleic acid inducing stop codon read-through have a delta G of 5 kcal/mole; 10 kcal/mole; 15 kcal/mole; 20 kcal/mole; 25 kcal/mole; 30 kcal/mole lower than non-cancer stop codon read-through counterparts. In some embodiments, the first 25, 50, or 75 nucleotides of the 3′UTR of a nucleic acid inducing stop codon read-through have a delta G in the range of 5 kcal/mole to 20 kcal/mole; 5 kcal/mole to 10 kcal/mole; or 10 kcal/mole to 20 kcal/mole; 25 kcal/mole; 30 kcal/mole lower than non-cancer stop codon read-through counterparts.

In some embodiments, aminoglycosides (e.g., gentamicin) and macrolides (e.g. erythromycin) can induce stop codon read-through. Without wishing to be bound by any theory, aminoglycosides can induce stop codon read-through by binding 18s rRNA and macrolides can induce stop codon read-through by binding the peptide channel within large ribosomal subunit. In some embodiments, aminoglycosides and macrolides can induce stop codon read-through in healthy (normal) cells. In some embodiments, subjects treated with aminoglycosides or macrolides should not be treated with a nucleic acid comprising a stop codon read-through motif.

In some embodiments, the present disclosure encompasses the recognition that an oncoselective translation sequence element can be oncospecific and result in translation and payload expression only in cancer cells (i.e., no detectable expression in non-cancer cells). Alternatively or additionally, in some embodiments, an oncoselective translation sequence element is translated 2, 5, 10, 15, 20, 30 or more—fold higher in cancer cell(s) as compared with appropriately comparable non-cancer cells.

In some embodiments, an oncoselective translation sequence element can comprise an internal ribosome entry segment/site (IRES). In some embodiments, an oncogenic ribosome, or RNA binding protein, preferentially binds an IRES in an oncoselective translation sequence element. In some embodiments, an oncoselective translation sequence element can be bound by or direct the binding of translation initiating RNA binding proteins (RBPs). In some embodiments, an oncoselective translation sequence element can comprise and IRES and be bound by or direct the binding of RBPs.

In some embodiments, an oncoselective read-through motifs is one listed in Table 6. In some embodiments, a putative oncoselective read-through motifs is one listed in Table 7.

TABLE 6
Exemplary nucleic acid sequences of oncoselective read-through motifs

| | | | SEQ |
|---|---|---|---|
| | | | ID |
| | ENS | Sequence | NO |
| U1 | 380715 | AAACGCAAGCAAGAGGAACAAATGGAAACTGAGCATTTTGCTCTGTAAAGCATTTCAGATGCAGCTT | 551 |
| | | GTGTG | |
| U2 | 428608 | GCGCTGACCCAGCTGATCCAGCTCTATCATCGCTTCCACCGGGTGCTGTCCCAGCCGCAGCTCCGAG | 552 |
| | | CCCTCCCTGCCCGGGCTGAGCTCATCAACATTCACCACCTTATGGTGGAGCTCAAGAAGCATAAGCC | |
| | | CAACTTCTGATGTGCCAGAAACCGCCCTGAGATCTGCCGGTCATCTCCATGGACTTCTGCACCCCAT | |
| | | TCCATACCCTTCTTCACCTGGGGTACCCCTTCCAGTTTTCCCCTTGCTTCCCAGGCCCTTGACATGG | |
| | | CTTACCTGCCTTCACTCCCAGCACCTTGCCCAACAGGA | |
| U3 | 409841 | CTTCCAGCAGAGGAGAACATGTCTAACACGTGCCTCAAAAGCACTGGGGAGTTAGTAGTGCAGTGGC | 553 |
| | | ATCTGAAACCTGTGGAGCAGAAAGCACATGAGTCCTAATGCCCCAGCAGCTTCCGATTG | |
| U4 | 274358 | ATAGTATACCCTGGAATTGCTGTATTTTTCCAGAATGCCTTCATCTTTTTTGGAGGGCTGGTTTTTA | 554 |
| | | AGTTTGGCCGCACTGAAGACTTATGGCAGTGAACACATCTGATTTCCCACAGCACAACAGCCCTGCA | |
| | | TGGGTTTGTTTGTTTTTTTACTGCTCACTCCCAACCTTTTGTAATGCCATTTTCTAAACTTATTTCT | |
| | | GAGTGTAGTCTCAGCTTAAAG | |
| U5 | 425191 | GGTGACAAATCCATTCGTTTCCGTCCCACGCTGGTCTTCAGGGATCACCACGCTCACCTGTTCCTCA | 555 |
| | | ATATTTTCAGTGACATCTTAGCAGACTTCAAGTAAAGAAGCCATTTCCACTACAGTGAGAAAGCCCG | |
| | | GATCCCAACAGTTGTCAAATTGATTAGTTTGCCTAATTCATGTTTTCACTTAAAAGTATCAGAGGTG | |
| U6 | 332780 | TGTCCTTTGGGCTCTGTGGAGAGCTTTAGCCTTGCACGGCGGCGCTGGGAGGCATTGCCTGCCATGC | 556 |
| | | CCACTGCCCGCTGCTCCTGCTCTAGTCTGCAGGCTGGGCCCCGGCTGTTTGTTATTGGGGGTGTGGC | |
| | | CCAGGGCCCCAGTCAAGCCGTGGAGGCACTGTGTCTGCGTGATGGGGTCTGAAGGCTTGGTGGGAGC | |
| | | TGTCCACTGGAGCAGCTCATTGCCAGAGGCAGCTATTTCTATGGCTCCTTTTGCTGCTGAGGA | |
| U7 | 360534 | ATATATACCCACATGAGCCACTTCATAAAACAATGTTTCTCTTTACCTTAGCACCTCAAAATACCAT | 557 |
| | | GCCATTTAAAGCTTATTAAAACTCATTTTTGTTTTCATTATCTCAAAACTGCACTGTCAAGATGATG | |
| | | ATGATCTTTAAAATACACACTCAAATCAAGAAACTTAAGGTTACCTTTGTTCCCAAATTTCATACCT | |
| U8 | 450195 | GAGGTTCTCCAGGACCTTAGGTTTGATGCGGAATCTGCCGAGTGATGGCGGCTCCCCAGGGATGCGC | 558 |
| | | CGAGGGAGATGGGAAACGGGGCGGATGGCGCCCAGCCCAGCCCTAACTGCCAGCTGGCTGGGGTTGC | |
| | | GCCCCACTGCGCTGCTGACCTTCCTGCAGTTCCAGACACCTCCCACAATAAAGAGCTCCTCCTCTGT | |
| U9 | 337231 | GGTGAGGTCAAGGCAGGAGAGAAGAGCCTGAGCCAGCACGTGGAGGCCGTGGACAAGCGGCTGGAAC | 559 |
| | | AGAGCCAGCCCGAGTACAAGGCGCTCTTCAAAGAGATCTTCTCCAGGATCCAGAAGACCAAGGCTGA | |
| | | CATCAACGCCACCAAAGTCAAGACGCACAGCAGCAAGTGACCCTTCTCCGGCCTGCAGCCTCCC | |
| U10 | 358607 | GAGGCGATCAGCGAGGTTCTCCAGGACCTTAGGTTTGATGCGGAATCTGCCGAGTGATGGCGGCTCC | 560 |
| | | CCAGGGATGCGCCGAGGGAGATGGGAAACGGGGCGGATGGCGCCCAGCCCAGCCCTAACTGCCAGCT | |
| | | GGCTGGGGTTGCGCCCCACTGCGCTGCTGACCTTCCTGCAGTTCCAGACACCTCCCACAATAAAGAG | |
| | | CTCCTCCTCTGT | |

TABLE 7
Exemplary sequences of putative oncoselective read-through motifs

| | G-C | | |
|---|---|---|---|
| | Content | | |
| Construct | (%)* | Linear Motif* | Motif Sequence |
| U11 | 72 | VNNNNNNMNNMWK | GAGGTTCTCCAGGACCTTAGGT |
| | | (SEQ ID NO: 571) | TTGATGCGGAATCTGCCGAGTG |
| | | | ATGGCGGCTCCCCAGGGATGCG |
| | | | CCGAGGGAGATGGGAAACGGGG |
| | | | CGGATGGCGCCCAGCCCAGCCC |
| | | | TAACTGCCAGCTGGCTGGGGTT |
| | | | GCGCCCCACTGCGCTGCTGACC |
| | | | TTCCTGCAGTTCCAGACACCTC |
| | | | CCACAATAAAGAGCTCCTCCTC |
| | | | TGT; (SEQ ID NO: 581) |
| U12 | 58 | WGNNSNHNHDNNN | ATCCTAGCTTCGGTGCTGGCAG |
| | | (SEQ ID NO: 572) | TGTCCCAACAGGAATACCTAGA |
| | | | CAGTATGAAGAAAAACAAAGTG |
| | | | CACAGAGACCCGCCCCCAGACA |
| | | | AGAGTTGATGGAGACCCAGGGA |
| | | | TTGGACACCATCTCCCAACCCC |
| | | | AGTACTCCTGCTCTCCGGTGCC |
| | | | ACCTCACCTTCTTTGGCTTCTT |
| | | | CCCTCTTGCCTCCTTCTGTTCT |
| | | | TTC; (SEQ ID NO: 582) |
| U13 | 50 | MWBNNNNNNNNNN, | ACTGTTTCCTATGATCCTAGGA |
| | | VMNNWNKNNNNNN | AACCCACTGTGAAGAAAATTGC |
| | | (SEQ ID NO: 573) | CCCAATGATGGCCAAGACAATT |
| | | | AAAGCTTTCAAGAACAGATTCT |
| | | | CCCGACGATAAACTGAGGACTT |
| | | | GCCTTGGAAATGGAATCTGGGG |
| | | | AGGCAGGAATACAAGGACAGTG |
| | | | GGGGTTGGGGAATGGAATTCTA |
| | | | CAGGAGACTGGAGTCTTGCTTT |
| | | | GTG; (SEQ ID NO: 583) |
| U14 | 52 | VNNNNNNMNNMWK, MWBNN | TTGTCATGTGTACAGGAAATCA |
| | | NNNNNNNN, VMNNWNKNNN | GTGATGTGGTGCAGAGGTAGCC |
| | | NNN | ACTGTTAGCCTGGTGGGAAAAT |
| | | (SEQ ID NO: 574) | GCACACATTTCTGAGGGGAGAG |
| | | | GGAAAAGGACTTGTTTTCCTGT |
| | | | GTTCTTGTTTTCAGAAAATGAA |
| | | | AGACTCATACTTGAGTGTGTTT |
| | | | ATGTG; (SEQ ID NO: 584) |
| U16 | 60 | VMNNWNKNNNNNN | GTGCAGCACAAAAAGCCCGCCG |
| | | (SEQ ID NO: 575) | ACATCCCTCAGGGCTCCTTGGC |
| | | | CTACCTGGAGCAGGCATCTGCC |
| | | | AACATCCCTGCACCTCTGAAGC |
| | | | CAACGTGAGCAAAGGGCAGAGG |
| | | | CAGTTGGCCTATGAGTGGGCTG |
| | | | ATGCGTGAGGTTGGCCACACAT |
| | | | TCCTTCCTGTGGACTTGACATT |
| | | | TTGGAAGAACTCTTTGCCAGAT |
| | | | AATGAGTTCATTTTAGTTTTAT |
| | | | GCTCCCAT; (SEQ ID NO: |
| | | | 585) |
| U17 | 72 | DVHVNNNCWNNNB, VMNNW | TGGGGTCCCCAGTGGGGAATGA |
| | | NKNNNNNN | ACGGGTACTTCCTCATCGAGCG |
| | | (SEQ ID NO: 576) | CGGAAAGAACATGTGTGGCCTG |
| | | | GCTGCCTGCGCCTCCTACCCCA |
| | | | TCCCTCTGGTGTGAGCCGTGGC |
| | | | AGCCGCAGCGCAGACTGGCGGA |
| | | | GAAGGAGAGGAACGGGCAGCCT |
| | | | GGGCCTGGGTGGAAATCCTGCC |
| | | | CTGGAGGAAGTTGTGGGGAGAT |
| | | | CCACTGGGACCCCCAACATTCT |
| | | | GCCCTCACCTCTGTGCCCAGCC |
| | | | TGGAAACCTACAGACAAGGAGG; |
| | | | (SEQ ID NO: 586) |
| U22 | 28 | MWBNNNNNNNNNN | GAAGAAACAAAGAATAAACTCA |
| | | (SEQ ID NO: 577) | AAGCCGGCAATGCCCTGGCATT |
| | | | TTTGGGTCTTGAGAGAAAACAA |
| | | | TTTGAATGACTGAATTTACTAC |
| | | | AAAGGCAAACTTTCAAAAGGAT |
| | | | ATCTCTTTTTTGTTTCCAAATA |
| | | | TGTATCAACAGGTATCAACAAA |
| | | | ATCCTATTTTGAACTATTTTAC |
| | | | TCAGAAAAGAATATCCCAAATA |
| | | | TCCCAAATTATTCATAATAAAA |
| | | | ATGATTTGAAAGTGTTTTCATT |
| | | | CTTAAAA; (SEQ ID NO: |
| | | | 587) |
| U24 | 28 | MWBNNNNNNNNNN | TTTTACTGTAAGCTGTGTTCAC |
| | | (SEQ ID NO: 578) | TCTTTTATACAAATGAAGAAGT |
| | | | TGCAAAGAATACTCATTGCAGC |
| | | | AGCCTTCCTCATTATCAGAAAT |
| | | | TAAAGAAATTTCTGAATAAATT |
| | | | GGCAGAAGAACGCAGACAGAAG |
| | | | AAGGAAACTTAAGATGTGCAAG |
| | | | GAGATTTAAGGATTTCAAAGAA |
| | | | AATAAAGGTTCTTTGTTTT; |
| | | | (SEQ ID NO: 588) |
| U27 | 84 | MWBNNNNNNNNNN | AAGTGAGGCTCTCCTCCCGCCC |
| | | (SEQ ID NO: 579) | CGCCCCTCCCACGCCTCACCAG |
| | | | CCCCCCGCGCGCCCACCCTCCG |
| | | | GCGGGTGACAGCTCCGGGATCA |
| | | | GCAACCCTTCCTGCTGCTGCTA |
| | | | CTGCTGCTGCTGCTGCCGCCGC |
| | | | CGCCGCCGCCGCTGCCCTTGGG |
| | | | TCCCCCCGAGTCTCCGGGACTG |
| | | | CCCTCTCGACTGTCAGTGGGGC |
| | | | AGCCTCTCCGACTCTGCACCCG |
| | | | CCTCGACCTCCCCACCCGCTCC |
| | | | CACACCCCTGTGCCCTCATGTG |
| | | | GAGCCTAAGAGAACAGAACAGG |
| | | | CCGTGAAGCCAGCAGAGAAA; |
| | | | (SEQ ID NO: 589) |
| U28 | | VNNNNNNMNNMWK | CCCCGCCCTCCCAGTTTCCGCG |
| | | (SEQ ID NO: 580) | CGCCTCTTTGGCAGCTGGTCAC |
| | | | ATGGTGAGGGTGGGGGTGAGGG |
| | | | GGCCTCTCTAGCTTGCGGCCTG |
| | | | TGTCTATGGTCGGGCCCTCTGC |
| | | | GTCCAGCTGCTCCGGACCGAGC |
| | | | TCGGGTGTATGGGGCCGTAGGA |
| | | | ACCGGCTCCGGGGCCCCGATAA |
| | | | CGGGCCGCCCCCACAGCACCCC |
| | | | GGGCTGGCGTGAGGGTCTCCCT |
| | | | TGATCTGAGA; (SEQ ID |
| | | | NO: 590) |

In some embodiments, the at least one oncoselective sequence comprises an untranslated region (UTR). In some embodiments, the UTR is a 5′ UTR or a 3′ UTR. In some embodiments, the at least one oncoselective sequence comprises a nucleic acid sequence that is at least 75%, at least 80%, at least 85%, at least 90%, at least 95%, or at least 99% identical to any one of SEQ ID NOs: 1-97 or 101-146. In some embodiments, the at least one oncoselective sequence comprises a nucleic acid sequence that is at least 50 contiguous nucleotides, at least 100 contiguous nucleotides, or at least 150 contiguous nucleotides identical to any one of SEQ ID NOs: 1-75, 91-97, or 101-146; or at least 5 contiguous nucleotides identical to any one of SEQ ID NOs: 76-90. In some aspects, described herein is an oncoselective sequence, oncoselective modification, or an oncoselective motif comprising. In some embodiments, the oncoselective sequence or modification comprises a nucleic acid sequence of any one of the nucleic acid sequences of SEQ ID NOs: 1-97 or SEQ ID NOs: 101-146. In some embodiments, the at least one oncoselective sequence comprises the nucleic acid sequence that is at least 75% at least 80%, at least 85%, at least 90%, at least 95%, or at least 99/identical to any one of SEQ ID NOs: 1-8. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-8. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 9-75. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 91-97. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 101-146. In some embodiments, the at least one oncoselective sequence comprises a nucleic acid motif comprising a nucleic acid sequence that is at least 5 contiguous nucleotides identical to any one of SEQ ID NOs: 76-90. In some embodiments, the at least one oncoselective sequence comprises a nucleic acid motif comprising a nucleic acid sequence that is any one of SEQ ID NOs: 76-90.

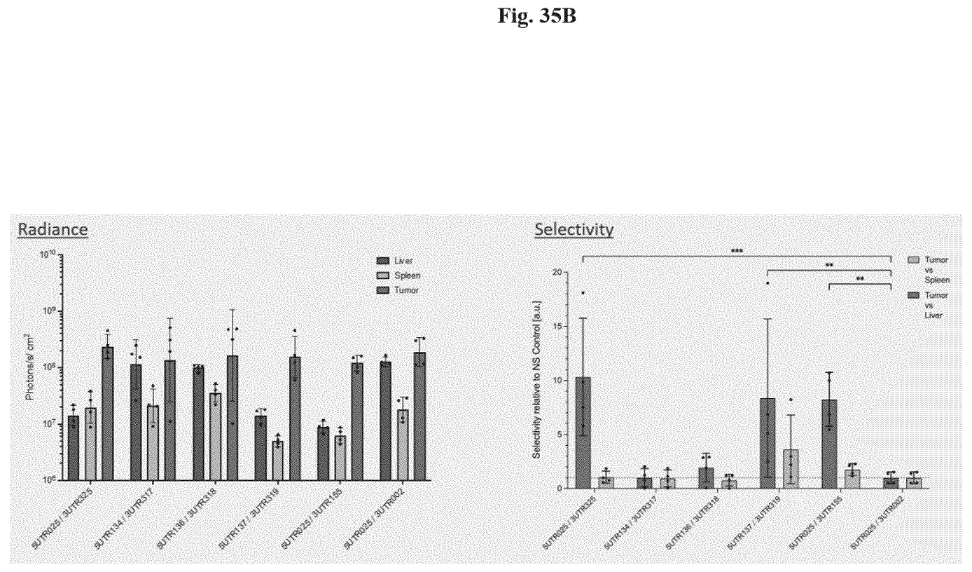

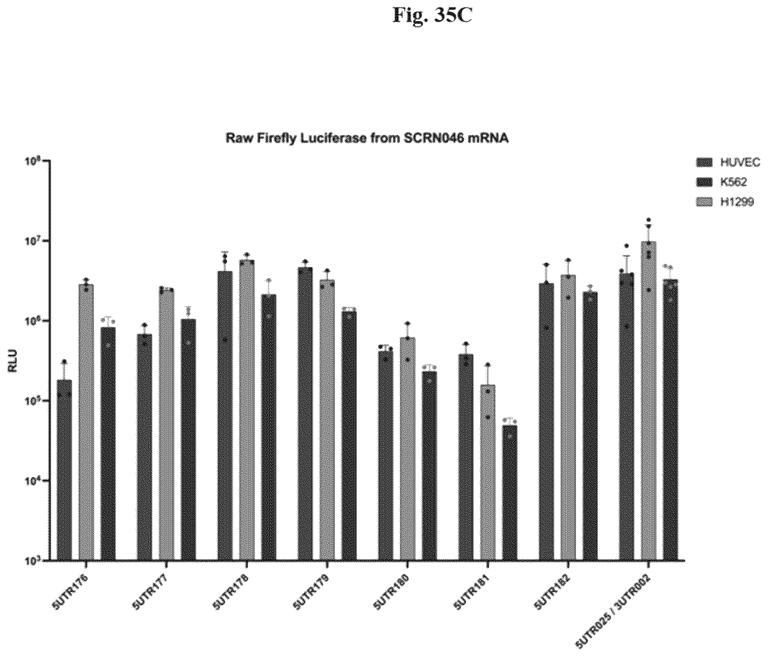

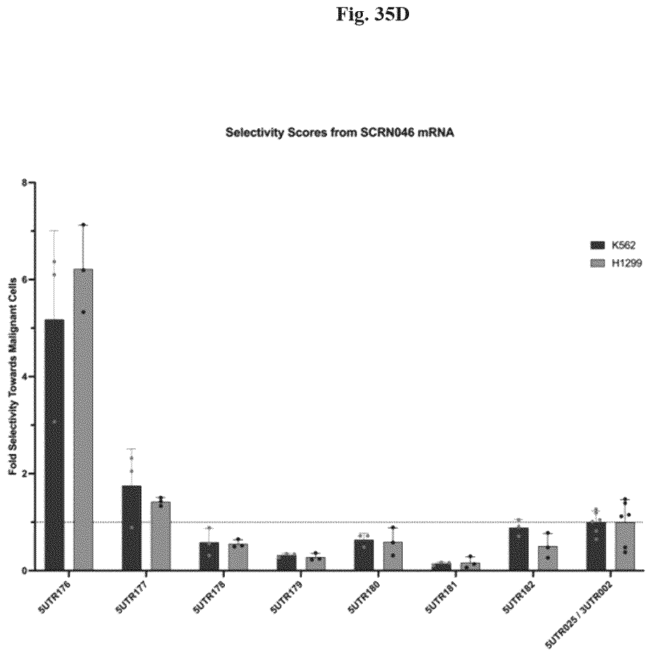

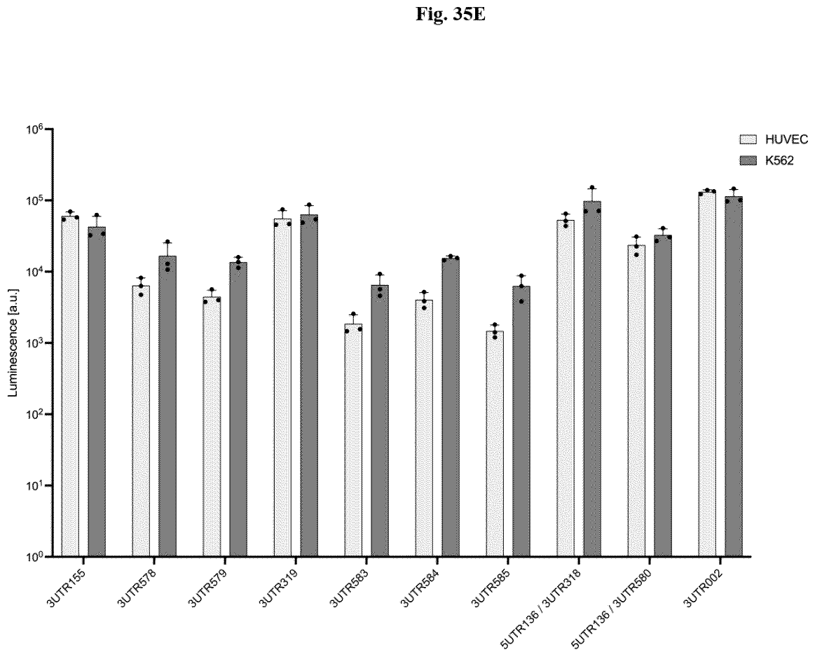

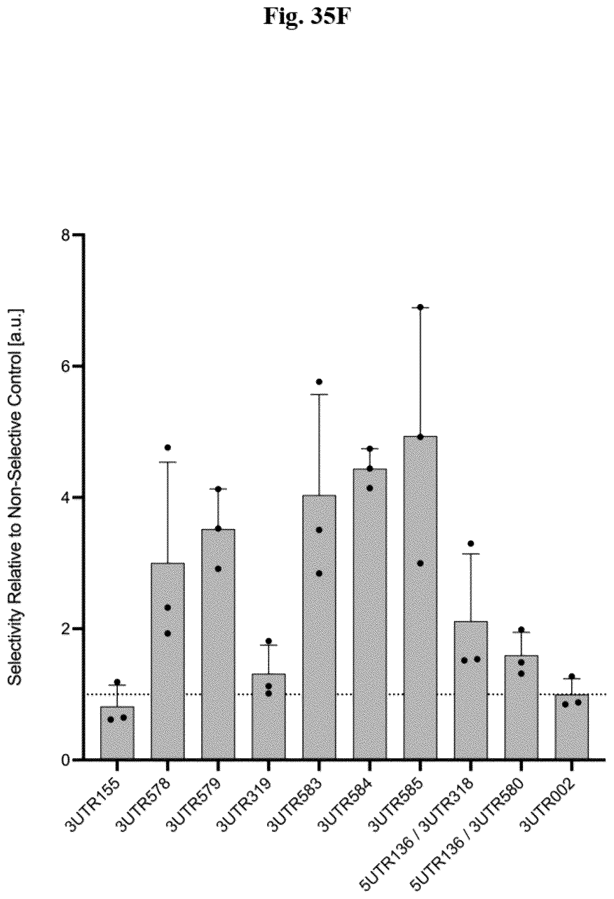

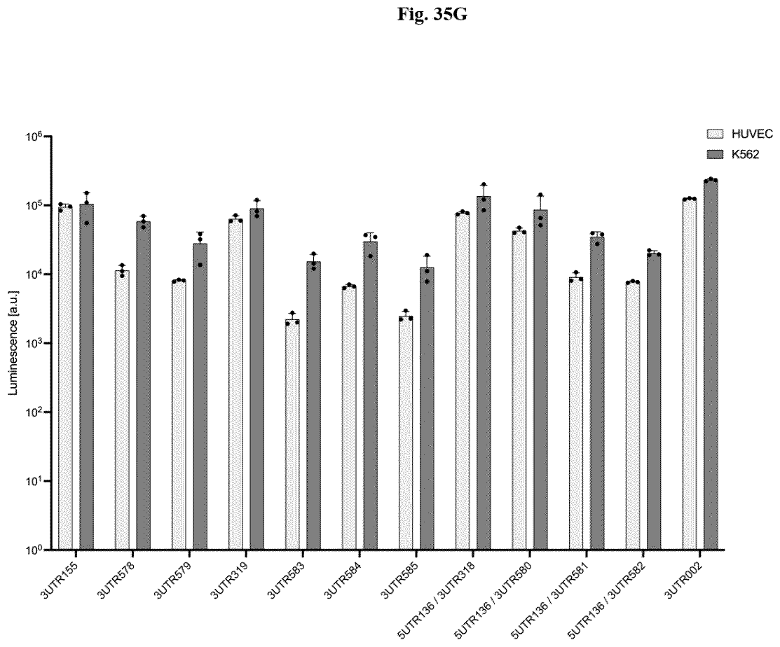

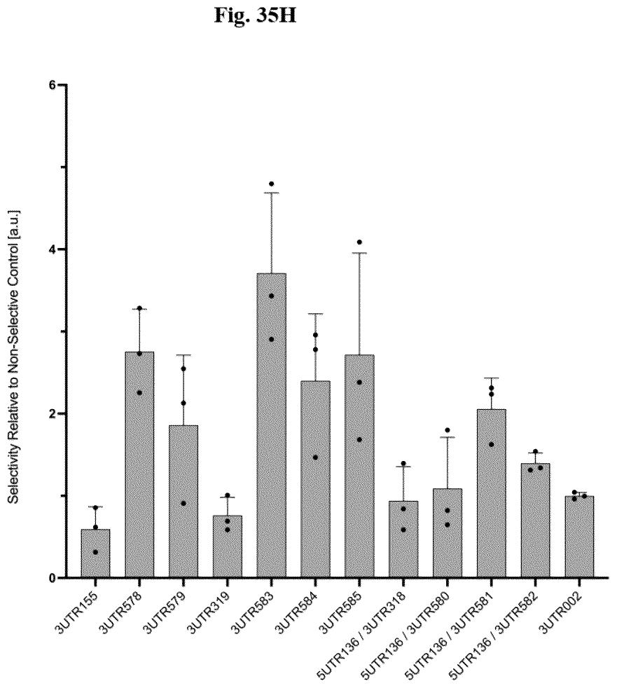

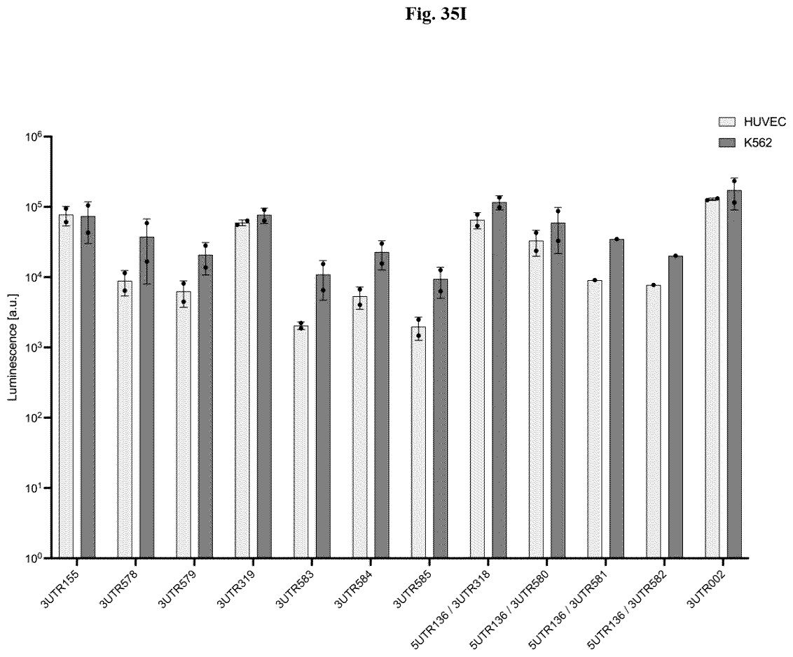

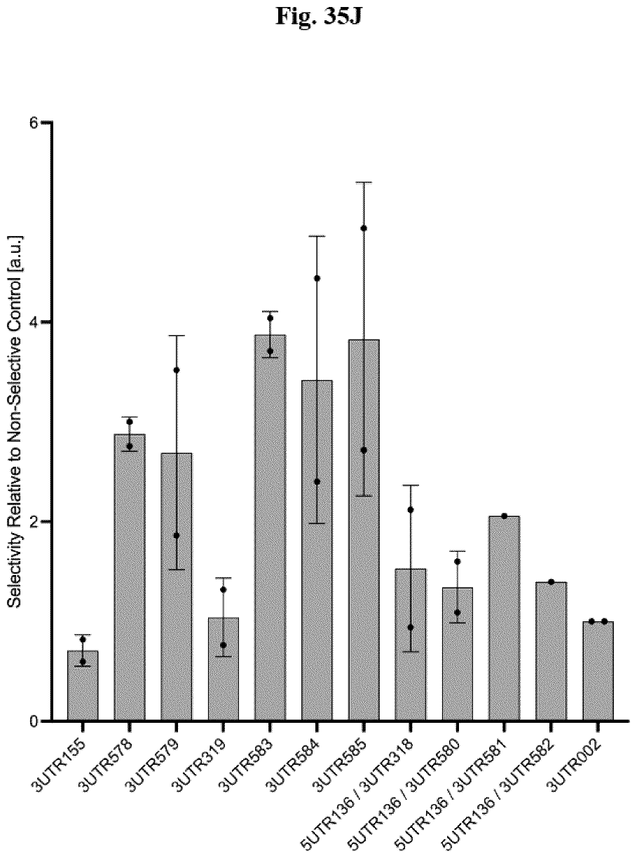

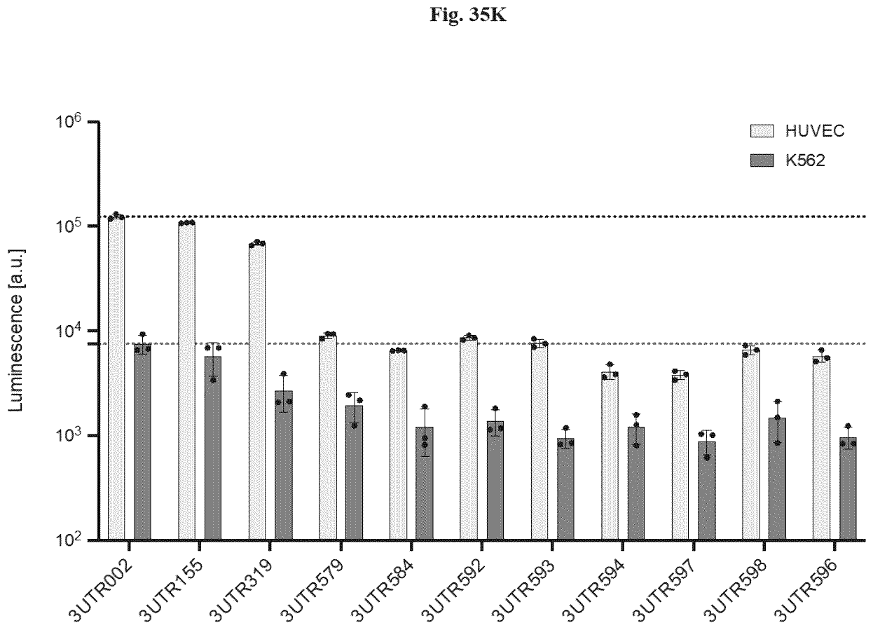

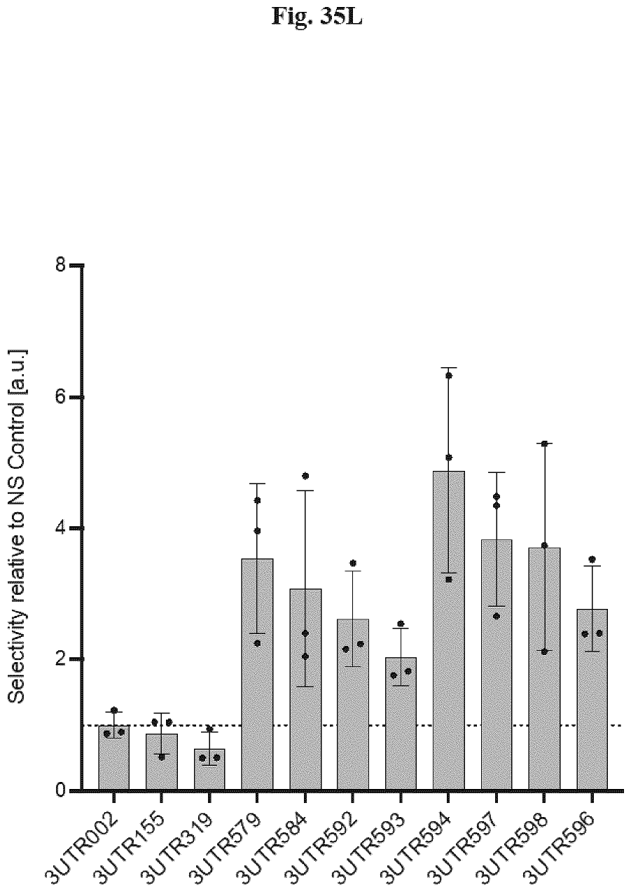

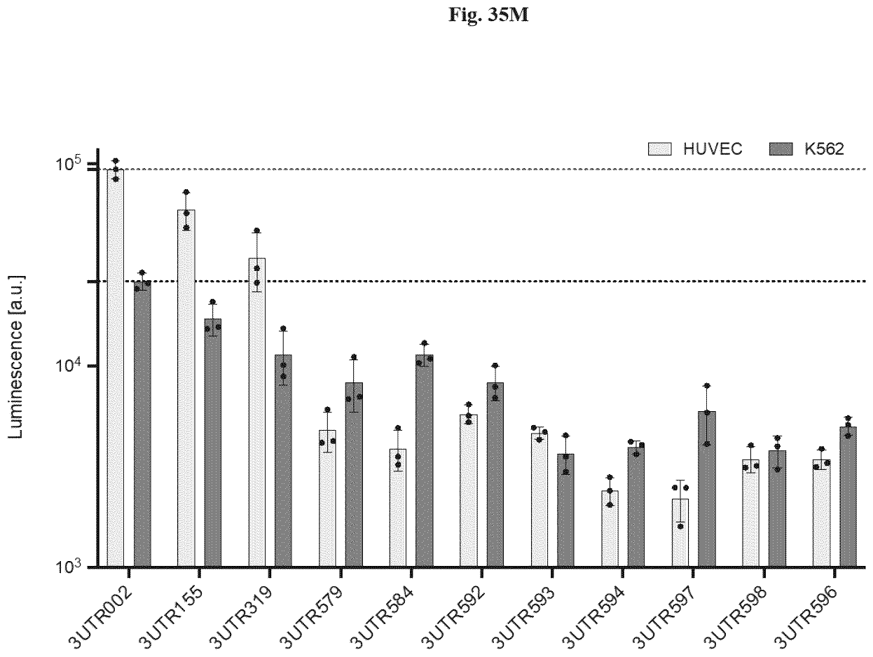

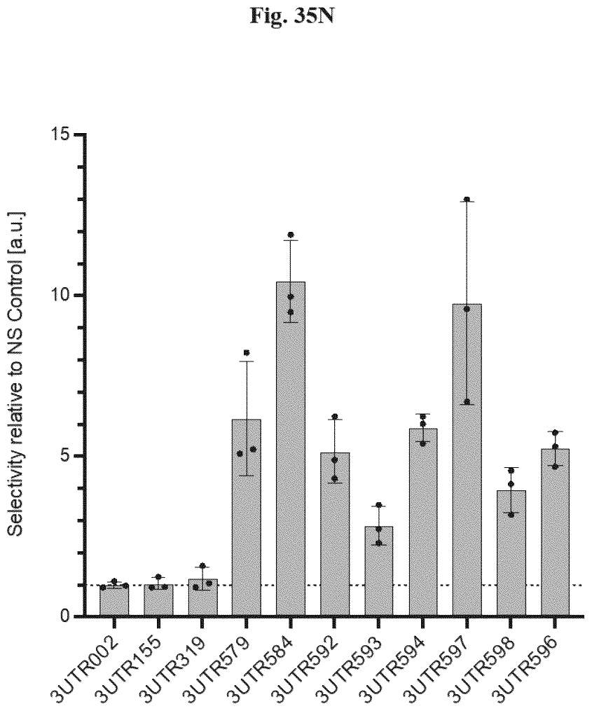

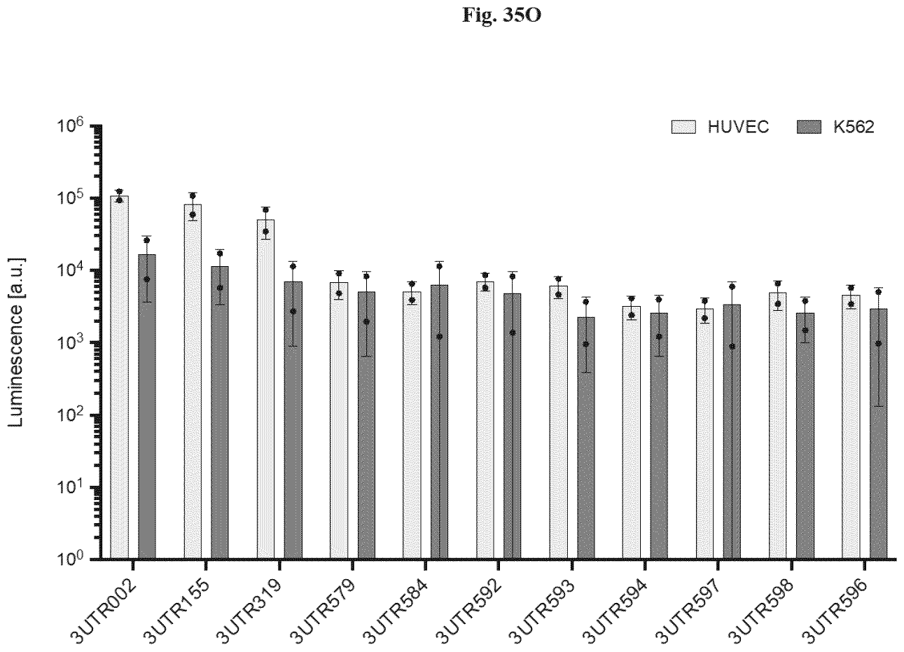

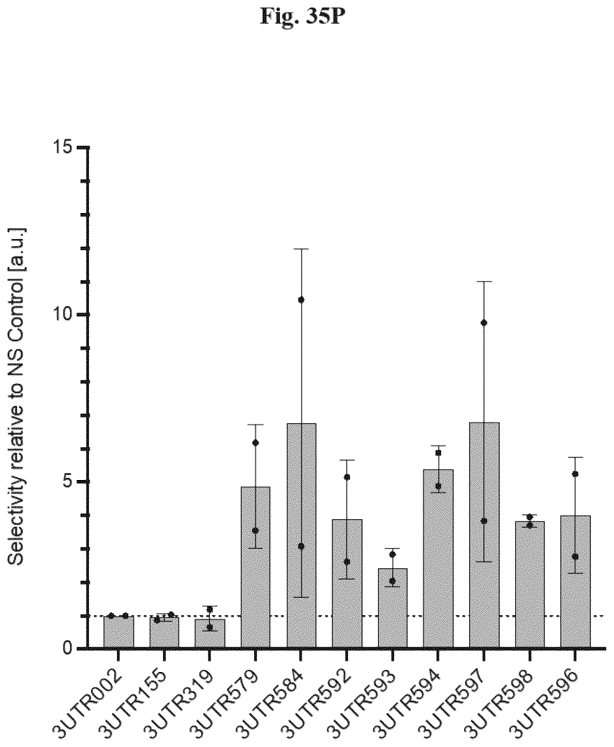

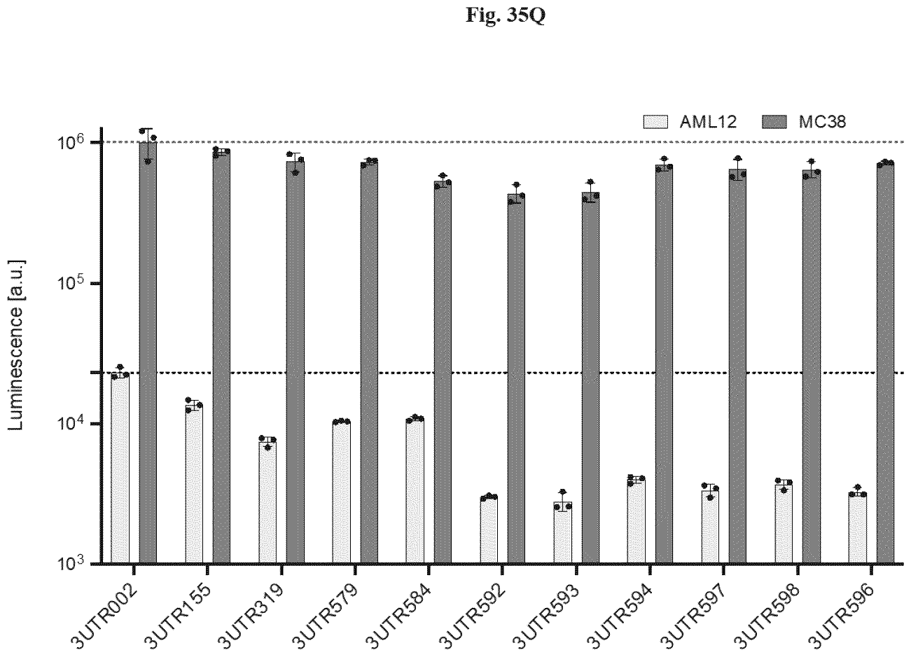

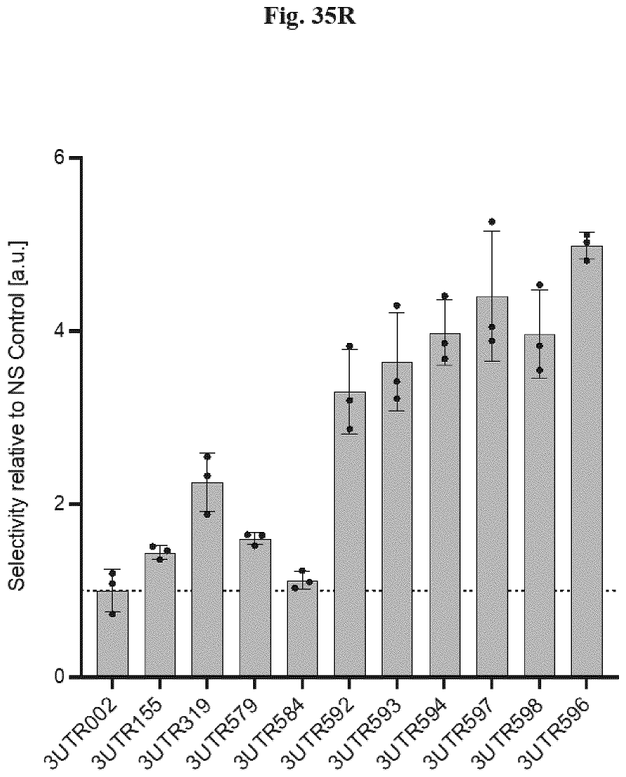

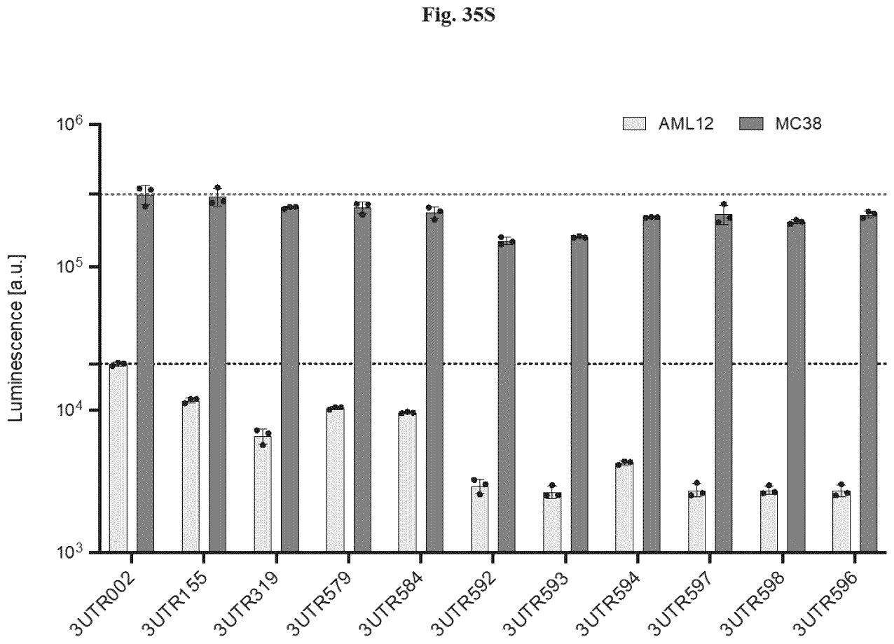

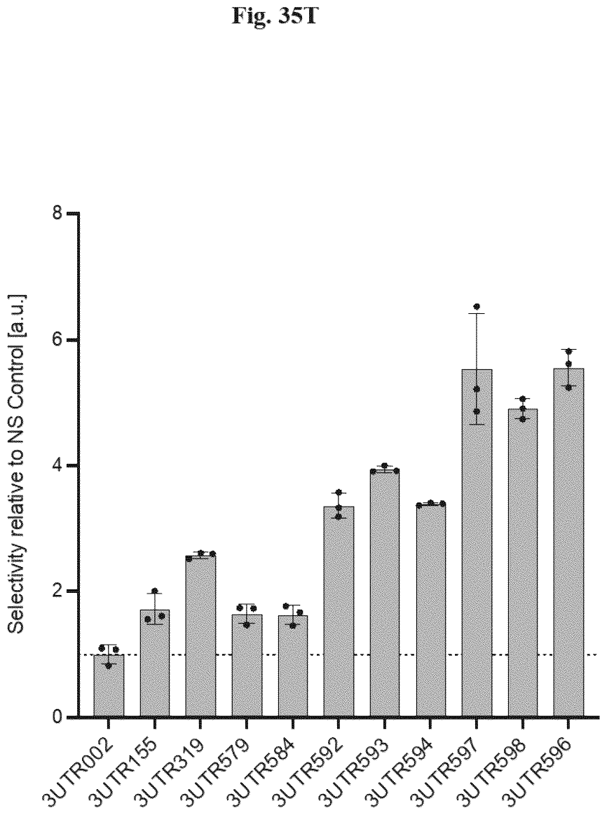

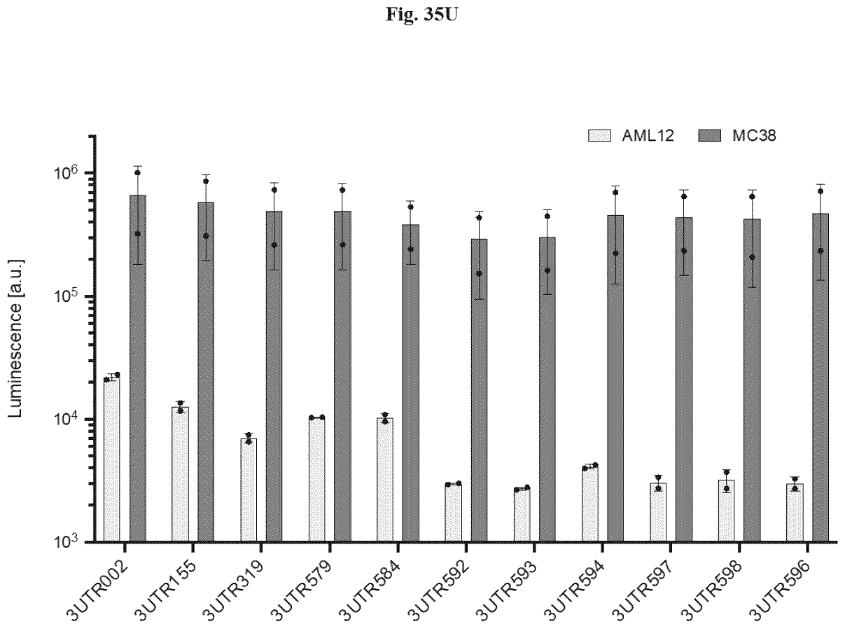

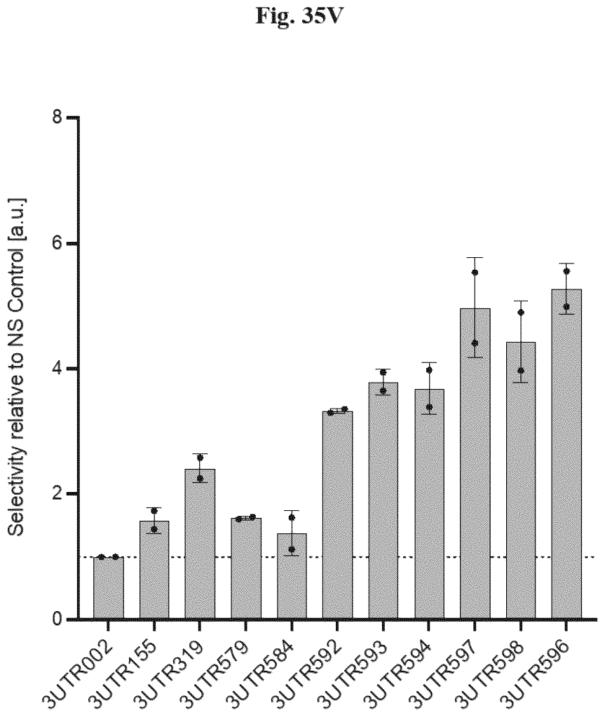

In some embodiments, the engineered polynucleotide comprises two oncoselective sequences. In some embodiments, the two oncoselective sequences flank the coding sequence. For example, FIG. 35A and FIG. 35B illustrate the increased expression driven by flanking a coding sequence with one oncoselective sequence at the 5′ end of the coding sequence and another oncoselective sequence at the 3′ end of the coding sequence. As shown in FIG. 35A and FIG. 35B, 3′-UTR sequence 3UTR155 (SEQ ID NO: 4) increased oncoselective expression in MC38 tumor model. Truncated version of 3UTR155, 3UTR325 (SEQ ID NO: 10) also increased oncoselective expression at a similar level as 3UTR155. The combination of 5UTR137 (SEQ ID NO: 93) and 3UTR319 (SEQ ID NO: 97) increased oncoselective expression in both B16F10 and MC38 tumor models. In some embodiments, the flanking oncoselective sequence at the 5′ end of the coding sequence can be a nucleic acid sequence that is at least 75%, at least 80%, at least 85%, at least 90%, at least 95%, or at least 99% identical to any one of SEQ ID NOs: 27 or 91-93. In some embodiments, the flanking oncoselective sequence at the 5′ end of the coding sequence can be a nucleic acid sequence that any one of SEQ ID NOs: 27 or 91-93. In some embodiments, the flanking oncoselective sequence at the 3′ end of the coding sequence can be a nucleic acid sequence that is at least 75%, at least 80%, at least 85%, at least 90%, at least 95%, or at least 99% identical to any one of SEQ ID NOs: 4, 10, or 94-97. In some embodiments, the flanking oncoselective sequence at the 3′ end of the coding sequence can be a nucleic acid sequence that any one of SEQ ID NOs: 4, 10, or 94-97. In some embodiments, the at least one oncoselective sequence comprises at least one miRNA binding site. In some embodiments, the at least one oncoselective sequence comprises at least one protein binding site. In some embodiments, the at least one protein binding site is a RNA binding protein (RBP) site.

### Engineered Polynucleotide

Among other things, the present disclosure provides nucleic acids (e.g., engineered polynucleotides) that participate in and/or are otherwise related to oncoselective translation as described herein. In some embodiments the present disclosure provides nucleic acids that are or include or deliver a translatable nucleic acid comprising an oncoselective sequence. In some embodiments, the present disclosure provides nucleic acids that are or include or deliver a translatable nucleic acid encoding a payload of interest and including an oncoselective translation sequence element as described herein.

In some embodiments, a provided engineered polynucleotide may be or comprise DNA (e.g., single or double-stranded DNA), e.g., that, when introduced into a cell, is transcribed, or generates a template strand that is transcribed) to produce a translatable nucleic acid (e.g., an RNA such as an mRNA) as described herein. In some embodiments, a provided engineered polynucleotide may be or comprise RNA (e.g., mRNA), which may be or comprise (or may be or comprise the complement of) a translatable nucleic acid described herein (e.g., may be or comprise a coding sequence and an oncoselective translation sequence element(s)).

In some embodiments, a provided nucleic acid is or comprises DNA or RNA or both. In some embodiments, a provided nucleic acid is chemically modified relative to naturally occurring DNA and/or RNA. In some embodiments, a provided nucleic acid is not modified with pseudouridine.

In some embodiments, a provided nucleic acid is a translatable nucleic acid as described herein. In some embodiments, a provided nucleic acid is expressible (e.g., can be transcribed to express) to produce a translatable nucleic acid as described herein. In some embodiments, a provided nucleic acid is a complement of a translatable nucleic acid as described herein, or of a nucleic acid that is expressible to produce such a translatable nucleic acid (or its complement).

Thus, in some embodiments, the present disclosure builds upon and enhances recent developments in the field of RNA (e.g., mRNA) therapeutics. Several groups have done important work developing technologies for, for example, improving RNA production and/or stability; providing encapsulating or other systems to facilitate RNA administration and/or delivery to mammalian (e.g., human) subjects; etc. Recent work by companies such as BioNTech AG, CureVac AG, Ethris AG, Moderna, Translate Bio, Inc., and others have led to development of several clinical candidates, and, recently, the first siRNA engineered therapeutic and mRNA vaccine approved by the US Food and Drug Administration; those skilled in the art will appreciate that any or all of the available technologies for production, stability, administration, etc. RNA engineered therapeutics may be applicable to and/or utilized with those embodiments of the present disclosure that administer a translatable RNA to mammalian (e.g., human) subjects.

Thus, in some embodiments, the present disclosure builds upon and enhances recent developments in the field of RNA (e.g., mRNA) therapeutics. Several groups have done important work developing technologies for, for example, improving RNA production and/or stability; providing encapsulating or other systems to facilitate RNA administration and/or delivery to mammalian (e.g., human) subjects; etc.

In some embodiments, a provided nucleic acid is engineered to show low or reduced (relative to an appropriate reference) immunogenicity when introduced, produced, and/or expressed in a subject. Those skilled in the art are aware of certain sequence elements and/or chemical modifications that can increase or decrease immunogenicity of a nucleic acid that contains them as compared with one that does not. In many embodiments, provided nucleic acids are engineered so that those that are or will be introduced, produced, and/or expressed in a subject are characterized by low expected or observed immunogenicity. For example, provided mRNAs can be engineered by increasing G-C content. The provided mRNAs can be modified by incorporation of non-canonical nucleotides, such as pseudouridine, N1-methylpseudouridine, methoxy-uridine, and 2-thiouridine into mRNA.

Alternatively or additionally, in some embodiments, a provided nucleic acid that includes or encodes a translatable payload is engineered so that the payload, when introduced and/or produced in a subject, shows relatively low immunogenicity. For example, in some embodiments, immunogenic epitope(s) may have been defined for a particular payload, and a less-immunogenic variant (e.g., having a sequence alteration within, or that otherwise impacts immunogenicity of such as by altering a pattern of post-translational modification, one or more such immunogenic epitope(s)) may be utilized in accordance with the present disclosure.

In some embodiments, a nucleic acid (e.g., an RNA) may be sequence engineered, for example to remove immunogenic sequence motifs. In some embodiments, a nucleic acid is sequence engineered to remove TLR7 or TLR8 stimulation motifs. In some embodiments, a nucleic acid is sequence engineered to remove motifs selected from the group consisting of KNUNDK motifs, UCW motifs, UNU motifs, UWN motifs, USU motifs, KWUNDK motifs, KNUWDK motifs, UNUNDK motifs, KNUNUK motifs, and combinations thereof.

### Coding Sequence

As described herein, the present disclosure relates particularly to translatable nucleic acids that comprise a coding sequence (e.g., a payload coding sequence) and an oncoselective translation sequence element.

Those of ordinary skill in the art, reading the present disclosure, will appreciate that a wide variety of useful payload sequences are known and can be utilized in accordance with the teachings herein. In some embodiments, the payload is a gene product (e.g., a polypeptide) that, when expressed in cancer cells, reduces their ability to survive and/or to proliferate within a subject.

In some embodiments, a payload sequence may be toxic to cells and/or may generate (e.g., enzymatically) a toxic agent. In some embodiments, the payload sequence encodes an engineered therapeutic described herein.

In some embodiments, a payload sequence may render cells more susceptible to immunological attack and/or clearance. For example, in some such embodiments, a payload sequence may be or comprise an antigen, antibody, antibody fragment, or their chimeric versions fused to a transmembrane protein and/or an intracellular signaling molecule (e.g. ITAM or costimulatory molecule endodomains) that is particularly attractive to a subject's immune system and/or to an immunological therapy (e.g., CAR-T or CAR-NK cells, proliferated T-cells, etc) that has been or will be administered to the subject. Alternatively or additionally, in some such embodiments, a payload sequence may be or comprise an agent that relieves or inhibits an immunological checkpoint.

As noted herein, one feature of the provided disclosure is that it achieves an extent of oncoselectivity such that payloads that would be unacceptable and/or inadvisable without such oncorestricted expression may be effectively utilized.

In some embodiments, a payload sequence for use in accordance with the present disclosure is selectively active in cancer cells and/or under particular circumstances (e.g., in the presence of a separate agent). However, in some embodiments, particularly in light of the degree of oncoselectivity provided by the present disclosure, in some embodiments, a payload comprises a protein that is constitutively active and/does not require post-translational modifications such as cleavage or phosphorylation.

In some embodiments, a payload is not secreted from a cell in which it is produced (e.g., by translation). In some other embodiments, a payload is a protein that is secreted into the tumor microenvironment.

In some embodiments, a polypeptide payload may be or comprise an antibody, a cell surface protein (e.g., that is or comprises an antigen or epitope targeted by endogenous or administered immune cells—such as T cells, NK cells, etc), an enzyme, a genetic modification protein, a suicide protein, a toxin, a viral replication protein, a viral surface antigen, etc. In some embodiments, a polypeptide payload may be or comprise a biologic agent approved for treatment of cancer.

In some embodiments a linker may be present between an oncoselective translation sequence element and a payload sequence. In some embodiments, a linker comprises 2A linker. In some embodiments, a linker comprises a PT2A linker. In some embodiments, a linker comprises a F2Am linker.

### Antibody Agents

Several antibody therapeutics useful in the treatment of cancer are known in the art. Recent developments in the mRNA engineered therapeutic field indicate that delivery of a translatable nucleic acid encoding an antibody agent of interest can be a viable and effective strategy for administering antibody therapeutics (see, for example, Van Hocke & Roose, J. Translational Med. 17:54, Feb. 22, 2019). Those skilled in the art, reading the present disclosure, will appreciate that its teachings are applicable to therapeutic antibody agents; in some embodiments, a translatable nucleic acid as described herein encodes a polypeptide that is or is a component of an engineered therapeutic antibody agent.

Described herein, in some aspects, is an engineered polynucleotide. In some embodiments, the engineered polynucleotide comprises a coding nucleic acid sequence, where the coding nucleic acid sequence encodes an engineered therapeutic or an engineered therapeutic described herein. In some embodiments, the engineered polynucleotide comprises at least one oncoselective sequence described herein. In some embodiments, the engineered polynucleotide comprises ribonucleic acid. In some embodiments, the engineered polynucleotide comprises an mRNA. In some embodiments, the engineered polynucleotide comprises at least one untranslated region. In some embodiments, the at least one untranslated region is a 3′-UTR. In some embodiments, the at least one untranslated region is a 5′ untranslated region a 5′-UTR. In some embodiments, the engineered polynucleotide comprises both a 3′-UTR and a 5′-UTR. In some embodiments, the engineered polynucleotide comprises at least one nucleic acid modification. For example, the engineered polynucleotide can comprise at least one nucleotide analogue. In some embodiments, the engineered polynucleotide comprises a degenerative sequence. For example, the engineered polynucleotide can comprise at least degenerative codon. Table 8 illustrates an example nomenclature denoting the identity of the nucleotide or degenerative nucleotide.

TABLE 8
Nucleic acid code

| Nucleic acid code | Definition | Mnemonic |
|---|---|---|
| Y | C, T or U | Pyrimidines |
| X | Masked | |
| W | A, T, or U | Weak interaction |
| V | Neither T nor U | V comes after U |
| | (i.e. A, C, or G) | |
| U | U | Uracil |
| T | T | Thymine |
| S | C or G | Strong interaction |
| R | A or G | Purine |
| N | A C G T U | Nucleic acid |
| M | A or C | Bases with amino groups |
| K | G, T or U | Bases which are ketones |
| H | Not G | H comes after G |
| | (i.e., A, C, T, or U) | |
| G | G | Guanine |
| D | Not C | D comes after C |
| | (i.e. A, G, T, or U) | |
| C | C | Cytosine |
| B | Not A | B comes after A |
| | (i.e. C, G, T, or U) | |
| A | A | Adenine |
| — | Gap of indeterminate length | |

TABLE 9
miRNA binding sites in 3′ UTR155

| Target Rank | Target Score | miRNA Name | Regions (1-indexing) |
|---|---|---|---|
| 1 | 80 | hsa-miR-548c-3p | 124-131 |
| 2 | 75 | hsa-miR-3619-3p | 145-151 |
| 3 | 71 | hsa-miR-4323 | 255-262 |
| 4 | 63 | hsa-miR-3918 | 20-27 |
| 5 | 51 | hsa-miR-6720-5p | 197-204 |
| 6 | 51 | hsa-miR-6512-3p | 197-204 |

TABLE 10
Comparison between mouse 3′ UTR155 versus mouse miRNA

| Target | Target | | Region (1-indexed, |
|---|---|---|---|
| Rank | Score | miRNA Name | mouse) |
| 1 | 77 | mmu-miR-7040-3p | 297-303 |
| 2 | 69 | mmu-miR-3472 | 301-308 |
| 3 | 60 | mmu-miR-7038-5p | 134-141 |
| 4 | 56 | mmu-miR-1955-5p | 39-46 |
| 5 | 53 | mmu-miR-7018-5p | 283-289 |

In some embodiments, the engineered polynucleotide comprises a vector. In some embodiments, the engineered polynucleotide is comprises a viral vector. In some embodiments, the vector is an expression vector. In the context of an expression vector, the vector can be readily introduced into a host cell, e.g., a mammalian, bacterial, yeast, or insect cell by any known method. For example, the expression vector can be transferred into a host cell by physical, chemical, or biological means. Physical methods for introducing a polynucleotide into a host cell include calcium phosphate precipitation, lipofection, particle bombardment, microinjection, gene gun, electroporation, and the like. Methods for producing cells comprising vectors and/or exogenous nucleic acids are suitable for methods herein. One method for the introduction of a polynucleotide into a host cell is calcium phosphate transfection. Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Example viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, viral vector is a chimeric viral vector, comprising viral portions from two or more viruses. In additional instances, the viral vector is a recombinant viral vector.

In some embodiments, the engineered polynucleotide comprises or is operatively coupled to a heterologous sequence. In some embodiments, the heterologous sequence comprises an exon sequence, an intron sequence, an exon-intron junction, a splice acceptor-splice donor site, a start codon sequence, a stop codon sequence, a promoter site, an alternative promoter site, 5′ regulatory element, enhancer, 5′ UTR region, 3′ UTR region, poly adenylation site, or binding site of a polymerase, nuclease, gyrase, topoisomerase, methylase or methyl transferase, transcription factors, etc. In some embodiments, the heterologous sequence comprises an expression control sequence such as a promoter.

### Untranslated Region

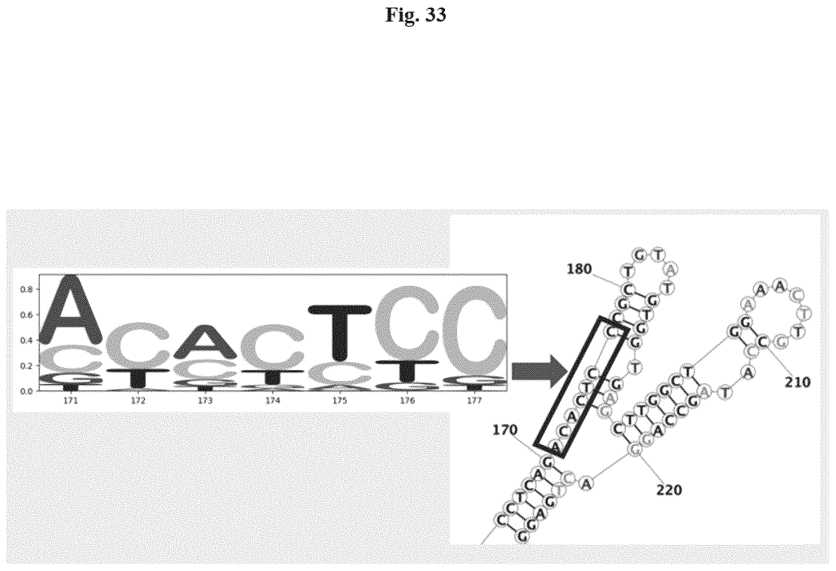

In some aspects, the engineered polynucleotide described herein comprises at least one untranslated region (UTR). In some embodiments, the engineered polynucleotide comprises a 3′-UTR. In some embodiments, the engineered polynucleotide comprises a 5′-UTR. In some embodiments, the engineered polynucleotide comprises both a 3′-UTR and a 5′-UTR. In some embodiments, the at least one UTR is a naturally occurring UTR. In some embodiments, the at least one UTR is a synthetic UTR or a heterologous UTR. In some embodiments, the at least one UTR comprises at least 10, at least 50, at least 100, at least 500, at least 1000, at least 5000, or at least 10000 nucleotides. In some embodiments, the at least one UTR comprises at least one nucleic acid structure such as a secondary structure or an RNA motif. In some embodiments, the secondary structure comprise a stem loop; a bulge loop, a pseudoknot, or a combination thereof. In some embodiments, the stem loop comprises a double stem loop (e.g., FIG. 33). In some embodiments, the at least one UTR does not yield a nucleic acid structure (e.g., the at least one UTR does not form an RNA motif).

### Nucleic Acid Modification

In some embodiments, the engineered polynucleotide described herein comprises at least one nucleic acid modification. In some embodiments, the at least one nucleic acid modification comprises substituting one or more nucleotide with one or more nucleotide analogues. In some embodiments, the nucleic acid modification comprises modifying A, G, U, or C ribonucleotides. In some embodiments, the modification can be made to a coding nucleic acid sequence of the engineered polynucleotide. In some embodiments, the modification can be made to a non-coding nucleic acid sequence of the engineered polynucleotide. In some embodiments, the modification can be made to both coding and non-coding nucleic acid sequence of the engineered polynucleotide. In some embodiments, the at least one nucleic acid modification increases resistant to degradation (e.g., hydrolysis or nuclease digestion) after in vivo administration of the engineered polynucleotide. In some embodiments, the at least one nucleic acid modification decreases immunogenicity after in vivo administration of the engineered polynucleotide as compared to a comparable nucleic acid sequence comprising a coding nucleic acid sequence that encodes an identical protein as the protein encoded by the engineered polynucleotide. In some aspects, the engineered polynucleotide, upon in vivo administration, increases expression of the protein encoded by the engineered polynucleotide compared to an expression of the same protein encoded by a comparable nucleic acid sequence. In some aspects, the engineered polynucleotide, upon in vivo administration, increases expression of the protein encoded by the engineered polynucleotide in a specific cell type compared to an expression of the same protein encoded by a comparable nucleic acid sequence in the same specific cell type.

In some embodiments, the at least one modification can be modification to 3′OH, group, 5′OH group, sugar, nucleobase, internucleotide linkage, or a combination thereof. Nucleic acid modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some aspects, chemically modified backbone comprises a backbone other than a phosphodiester backbone. In some aspects, a modified sugar comprises a sugar other than deoxyribose (in modified DNA) or other than ribose (modified RNA). In some aspects, a modified base comprises a base other than adenine, guanine, cytosine, thymine or uracil. In some aspects, the engineered polynucleotide comprises at least one chemically modified base. In some instances, the comprises at least one, two, three, four, five, six, seven, eight, nine, 10, 15, 20, 50, 100, or more modified bases. In some cases, nucleic acid modifications to the base moiety include natural and synthetic modifications of adenine, guanine, cytosine, thymine, or uracil, and purine or pyrimidine bases. For example, the nucleic acid modification comprises modifying at least one uracil of the engineered polynucleotide to 5′-methoxyuridine.

In some aspects, the at least one nucleic acid modification of the engineered polynucleotide comprises a modification of any one of or any combination of: 2′ modified nucleotide comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA); modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Non limiting examples of nucleic acid modification to the engineered polynucleotide can include: modification of one or both of non-linking or linking phosphate oxygens in the phosphodiester backbone linkage (e.g., sulfur (S), selenium (Se), BR3 (wherein R can be, e.g., hydrogen, alkyl, or aryl), C (e.g., an alkyl group, an aryl group, and the like), H, NR2, wherein R can be, e.g., hydrogen, alkyl, or aryl, or wherein R can be, e.g., alkyl or aryl); replacement of the phosphate moiety with “dephospho” linkers (e.g., replacement with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino); modification or replacement of a naturally occurring nucleobase with nucleic acid analog; modification of deoxyribose-phosphate or ribose-phosphate backbone (e.g., modifying the ribose-phosphate backbone to incorporate phosphorothioate, phosphonothioacetate, phosphoroselenates, boranophosphates, borano phosphate esters, hydrogen phosphonates, phosphonocarboxylate, phosphoroamidates, alkyl or aryl phosphonates, phosphonoacetate, or phosphotriesters; modification of 5′ end (e.g., 5′ cap or modification of 5′ cap-OH) or 3′ end of the nucleic acid sequence (3′ tail or modification of 3′ end-OH); substitution of the phosphate group with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino; modification of the ribophosphate backbone to incorporate morpholino (phosphorodiamidate morpholino oligomer PMO), cyclobutyl, pyrrolidine, or peptide nucleic acid (PNA) nucleoside surrogates; modifications to the sugar of a nucleotide to incorporate locked nucleic acid (LNA), unlocked nucleic acid (UNA), ethylene nucleic acid (ENA), constrained ethyl (cEt) sugar, or bridged nucleic acid (BNA); modification of a constituent of the ribose sugar (e.g., 2′-O-methyl, 2′-O-methoxyethyl (2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE)); modification to the base of a nucleotide (of A, T, C, G, or U); and stereopure of nucleotide (e.g., S conformation of phosphorothioate or R conformation of phosphorothioate).

In some aspects, the nucleic acid modification of the engineered polynucleotide comprises at least one substitution of one or both of non-linking phosphate oxygen atoms in a phosphodiester backbone linkage of the engineered polynucleotide. In some aspects, the at least one nucleic acid modification of the engineered polynucleotide comprises a substitution of one or more of linking phosphate oxygen atoms in a phosphodiester backbone linkage of the engineered polynucleotide. A non-limiting example of a nucleic acid modification of a phosphate oxygen atom is a sulfur atom. In some aspects, the nucleic acid modification of the engineered polynucleotide comprises at least one nucleic acid modification to a sugar of a nucleotide of the engineered polynucleotide. In some aspects, the nucleic acid modification of the engineered polynucleotide comprises at least one nucleic acid modification to the sugar of the nucleotide, where the nucleic acid modification comprises at least one locked nucleic acid (LNA). In some aspects, the nucleic acid modification of the engineered polynucleotide comprises at least one nucleic acid modification to the sugar of the nucleotide of the engineered polynucleotide comprising at least one unlocked nucleic acid (UNA). In some aspects, the nucleic acid modification of the engineered polynucleotide comprises at least one nucleic acid modification to the sugar of the nucleotide of the engineered polynucleotide comprising at least one ethylene nucleic acid (ENA). In some aspects, the nucleic acid modification of the engineered polynucleotide comprises at least one nucleic acid modification to the sugar comprising a modification of a constituent of the sugar, where the sugar is a ribose sugar. In some aspects, the nucleic acid modification of the engineered polynucleotide comprises at least one nucleic acid modification to the constituent of the ribose sugar of the nucleotide of the engineered polynucleotide comprising a 2′-O-Methyl group. In some aspects, the nucleic acid modification of the engineered polynucleotide comprises at least one nucleic acid modification comprising replacement of a phosphate moiety of the engineered polynucleotide with a dephospho linker. In some aspects, the nucleic acid modification of the engineered polynucleotide comprises at least one nucleic acid modification of a phosphate backbone of the engineered polynucleotide. In some aspects, the engineered polynucleotide comprises a phosphothioate group. In some aspects, the nucleic acid modifications of the engineered polynucleotide comprises at least one nucleic acid modification comprising a modification to a base of a nucleotide of the engineered polynucleotide. In some aspects, the nucleic acid modifications of the engineered polynucleotide comprises at least one nucleic acid modification comprising an unnatural base of a nucleotide. In some aspects, the nucleic acid modifications of the engineered polynucleotide comprises at least one nucleic acid modification comprising a morpholino group (e.g., a phosphorodiamidate morpholino oligomer, PMO), a cyclobutyl group, pyrrolidine group, or peptide nucleic acid (PNA) nucleoside surrogate. In some aspects, the nucleic acid modifications of the engineered polynucleotide comprises at least one nucleic acid modification comprising at least one stereopure nucleic acid. In some aspects, the at least one nucleic acid modification can be positioned proximal to a 5′ end of the engineered polynucleotide. In some aspects, the at least one nucleic acid modification can be positioned proximal to a 3′ end of the engineered polynucleotide. In some aspects, the at least one nucleic acid modification can be positioned proximal to both 5′ and 3′ ends of the engineered polynucleotide.

In some aspects, an engineered polynucleotide comprises a backbone comprising a plurality of sugar and phosphate moieties covalently linked together. In some cases, a backbone of an engineered polynucleotide comprises a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some aspects, a backbone of an engineered polynucleotide can lack a 5′ reducing hydroxyl, a 3′ reducing hydroxyl, or both, capable of being exposed to a solvent. In some aspects, a backbone of an engineered polynucleotide can lack a 5′ reducing hydroxyl, a 3′ reducing hydroxyl, or both, capable of being exposed to nucleases. In some aspects, a backbone of an engineered polynucleotide can lack a 5′ reducing hydroxyl, a 3′ reducing hydroxyl, or both, capable of being exposed to hydrolytic enzymes. In some instances, a backbone of an engineered polynucleotide can be represented as a polynucleotide sequence in a circular 2-dimensional format with one nucleotide after the other. In some instances, a backbone of an engineered polynucleotide can be represented as a polynucleotide sequence in a looped 2-dimensional format with one nucleotide after the other. In some cases, a 5′ hydroxyl, a 3′ hydroxyl, or both, are joined through a phosphorus-oxygen bond. In some cases, a 5′ hydroxyl, a 3′ hydroxyl, or both, are modified into a phosphoester with a phosphorus-containing moiety.

In some aspects, the engineered polynucleotide described herein comprises at least one nucleic acid modification. A nucleic acid modification can be a substitution, insertion, deletion, nucleic acid modification, physical modification, stabilization, purification, or any combination thereof. In some cases, a modification is a nucleic acid modification. Suitable nucleic acid modifications comprise any one of: 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, LNA, cEt, pseudouridine-5′-triphosphate, 5-methylcytidine-5′-triphosphate, 2-O-methyl-phosphorothioate or any combinations thereof.

In some cases, a modification can be permanent. In other cases, a modification can be transient. In some cases, multiple modifications are made to the engineered polynucleotide, the engineered polynucleotide modification can alter physio-chemical properties of a nucleotide, such as their conformation, polarity, hydrophobicity, chemical reactivity, base-pairing interactions, or any combination thereof.

In some embodiments, the phosphate group of a chemically modified nucleotide can be modified by replacing one or more of the oxygens with a different substituent. In some aspects, the chemically modified nucleotide can include replacement of an unmodified phosphate moiety with a modified phosphate as described herein. In some aspects, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the engineered polynucleotide comprises stereopure nucleotides comprising S conformation of phosphorothioate or R conformation of phosphorothioate. In some aspects, the chiral phosphate product is present in a diastereomeric excess of 50%, 60%, 70%, 80%, 90%, or more. In some aspects, both non-bridging oxygens of phosphorodithioates can be replaced by sulfur. The phosphorus center in the phosphorodithioates can be achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl). In some aspects, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some aspects, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; proengineered polynucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It is also understood in a nucleotide substitute that both the sugar and the phosphate moieties of the nucleotide can be replaced, by for example an amide type linkage (aminoethylglycine) (PNA). It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some aspects, the nucleic acid modification described herein comprises modification of a phosphate backbone. In some aspects, the engineered polynucleotide described herein comprises at least one chemically modified phosphate backbone. Example chemically modification of the phosphate group or backbone can include replacing one or more of the oxygens with a different substituent. Furthermore, the modified nucleotide present in the engineered polynucleotide can include the replacement of an unmodified phosphate moiety with a modified phosphate as described herein. In some aspects, the modification of the phosphate backbone can include alterations resulting in either an uncharged linker or a charged linker with unsymmetrical charge distribution. Example modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp). In such case, the chemically modified engineered polynucleotide can be stereopure (e.g. S or R confirmation). In some cases, the chemically modified engineered polynucleotide comprises stereopure phosphate modification. For example, the chemically modified engineered polynucleotide comprises S conformation of phosphorothioate or R conformation of phosphorothioate.

Phosphorodithioates have both non-bridging oxygens replaced by sulfur. The phosphorus center in the phosphorodithioates is achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

In some aspects, at least one phosphate group of the engineered polynucleotide can be chemically modified. In some aspects, the phosphate group can be replaced by non-phosphorus containing connectors. In some aspects, the phosphate moiety can be replaced by dephospho linker. In some aspects, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some aspects, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g. 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

In some aspects, the nucleic acid modification described herein comprises modification by replacement of a phosphate group. In some aspects, the engineered polynucleotide described herein comprises at least one chemically modification comprising a phosphate group substitution or replacement. Example phosphate group replacement can include non-phosphorus containing connectors. In some aspects, the phosphate group substitution or replacement can include replacing charged phosphate group can by a neutral moiety. Example moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

In some aspects, the nucleic acid modification described herein comprises modifying ribophosphate backbone of the engineered polynucleotide. In some aspects, the engineered polynucleotide described herein comprises at least one chemically modified ribophosphate backbone. Example chemically modified ribophosphate backbone can include scaffolds that can mimic nucleic acids can also be constructed wherein the phosphate linker and ribose sugar are replaced by nuclease resistant nucleoside or nucleotide surrogates. In some aspects, the nucleobases can be tethered by a surrogate backbone. Examples can include morpholino such as a phosphorodiamidate morpholino oligomer (PMO), cyclobutyl, pyrrolidine and peptide nucleic acid (PNA) nucleoside surrogates.

In some aspects, the nucleic acid modification described herein comprises modification of sugar. In some aspects, the engineered polynucleotide described herein comprises at least one chemically modified sugar. Example chemically modified sugar can include 2′ hydroxyl group (OH) modified or replaced with a number of different “oxy” or “deoxy” substituents. In some aspects, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR, wherein R can be, e.g., H or optionally substituted alkyl, and n can be an integer from 0 to 20. In some aspects, the “oxy”-2′ hydroxyl group modification can include (LNA, in which the 2′ hydroxyl can be connected, e.g., by a Ci-₆ alkylene or Cj-6 heteroalkylene bridge, to the 4′ carbon of the same ribose sugar, where example bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH2)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some aspects, the “oxy”-2′ hydroxyl group modification can include the methoxyethyl group (MOE), (OCH₂CH₂OCH₃, e.g., a PEG derivative). In some cases, the deoxy modifications can include hydrogen (i.e., deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the Γ position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that are in the L form, e.g. L-nucleosides. In some aspects, the engineered polynucleotide described herein includes the sugar group ribose, which is a 5-membered ring having an oxygen. Example modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some aspects, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid. In some aspects, the modifications to the sugar of the engineered polynucleotide comprises modifying the engineered polynucleotide to include locked nucleic acid (LNA), unlocked nucleic acid (UNA), ethylene nucleic acid (ENA), constrained ethyl (cEt) sugar, or bridged nucleic acid (BNA).

In some aspects, the engineered polynucleotide described herein comprises at least one nucleic acid modification of a constituent of the ribose sugar. In some aspects, the nucleic acid modification of the constituent of the ribose sugar can include 2′-O-methyl, 2′-O-methoxyethyl (2′-O-MOE), 2′-fluoro, 2′-aminoethyl, 2′-deoxy-2′-fuloarabinou-cleic acid, 2′-deoxy, , 2′-deoxy-2′-fluoro, 2′-O-methyl, 3′-phosphorothioate, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA) 3′-phosphonoacetate (PACE), or 3′-phosphonothioacetate (thioPACE). In some aspects, the nucleic acid modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some cases, unnatural nucleic acids include amide linked nucleoside dimers have been prepared for incorporation into engineered polynucleotides wherein the 3′ linked nucleoside in the dimer (5′ to 3′) comprises a 2′-OCH₃ and a 5′-(S)—CH. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some aspects, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R=H, C₁-C₁₂ alkyl or a protecting group); and combinations thereof.

In some instances, the engineered polynucleotide described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl. 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙONH₂, and —O(CH₂)ₙON[(CH2)nCH₃)]₂, where n and m are from 1 to about 10. Other nucleic acid modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an engineered polynucleotide, or a group for improving the pharmacodynamic properties of an engineered polynucleotide, and other substituents having similar properties. Similar modifications may also be made at other positions on the sugar, particularly the 3′ position of the sugar on the 3′ terminal nucleotide or in 2′-5′ linked engineered polynucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′—(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′ and analogs thereof.

In some aspects, the nucleic acid modification described herein comprises modification of the base of nucleotide (e.g. the nucleobase). Example nucleobases can include adenine (A), thymine (T), guanine (G), cytosine (C), and uracil (U). These nucleobases can be modified or replaced to in the engineered polynucleotide described herein. The nucleobase of the nucleotide can be independently selected from a purine, a pyrimidine, a purine or pyrimidine analog. In some aspects, the nucleobase can be naturally-occurring or synthetic derivatives of a base.

### Immune Checkpoint Inhibitors and Modulators

Immune checkpoint are the regulators of immune system. They play a significant role in the immune evasion and escape of human tumors. Their modulators have shown significant efficacy in the cancer therapeutics field. When secreted from tumors the intratumoral concentrations of such immune modulators are higher and their systemic concentrations are lower. This improved pharmacokinetic profile can boost the efficacy and lower the toxicity associated with these agents. In some embodiments, a payload may be or comprises an immune checkpoint inhibitor, i.e. an antagonist antibody agent against immune checkpoint proteins, e.g. anti-PDI, anti-PDL1, anti-CTLA-4, anti-TIM3, anti-BTLA, anti-VISTA, anti-LAG-3, anti-TIGIT, anti-CD39, anti-SIRP-α. In some other embodiments, a payload may be or comprises an agonist antibody against CD-28, OX40, GITR, CD137, CD27, HVEM, or CD27. In some other embodiments, the payload may be a costimulatory molecule such as CD80, CD86, and OX40L.

### Cytokines

Cytokines have critical roles in regulation of immune cells. IL-2 and IFN-alpha were the first two immunotherapy cytokines that were FDA approved for the treatment of metastatic melanoma and renal cell carcinoma (high dose, bolus Il-2) and Stage III melanoma (IFN-alpha). However, their clinical use is limited by systemic toxicity issues (Rosenberg, J Immunol, 2014, 192 (12) 5451-5458). Those skilled in the art will appreciate that onco-selective production and secretion of cytokines can greatly improve their therapeutic window. In some embodiments, a payload for use in accordance with the present disclosure may be IL-2, IL-2 superkine/mutein, IL-12, IL-15, IL-15, IL-15R-alpha fusion, 11-23, IL-36, TNF-alpha, IFN-alpha, IFN-gamma, FLT3 ligand, CCL4, RANTES, GM-CSF, or engineered variants or fusions thereof.

In some aspects, described herein is a composition or method utilizing at least one mRNA, where the mRNA encodes at least one cytokine (e.g., at least one interleukin or at least one interferon). Non-limiting example of cytokine encoded by the mRNA described herein can include 4-1BBL, acylation stimulating protein, adipokine, albinterferon, APRIL, Arh, BAFF, Bcl-6, CCL1, CCL1/TCA3, CCL11, CCL12/MCP-5, CCL13/MCP-4, CCL14, CCL15, CCL16, CCL17/TARC, CCL18, CCL19, CCL2, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CCR10, CCR3, CCR4, CCR5, CCR6, CCR7, CCR8, CD153, CD154, CD178, CD40LG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL11, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAMI9A3, FAM19A4, FAMI9A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFNA5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17 family, IL-17A-F, IL-18, IL-18BP, IL-19, IL-1A, IL-1B, IL-1F10, IL-1F3/IL-1RA, IL-1F5, IL-1F6, IL-1F7, IL-1F8, IL-1F9, IL-1-like, IL-1RA, IL-1RL2, IL-1α, IL-1β, IL-2, IL-20, IL-21, 11-22, IL-23, IL-24, IL-28A, IL-28B, IL-29, IL-30, IL-31, IL-32, IL-33, IL-34, IL-35, IL-36, IL-37, IL-38, IL-39, or IL-40, IL-4, IL-5, IL-6, IL-6-like, IL-7, IL-8/CXCL8, IL-9, inflammasome, interferome, interferon alpha (IFN-α), interferon beta-1a, interferon beta-1b, interferon gamma, interferon type I, interferon type IL, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MHC class III, miscellaneous hematopoietins, monokine, MSP, myokine, myonectin, nicotinamide phosphoribosyltransferase, oncostatin M (OSM), oprelvekin, OX40L, platelet factor 4, promegapoietin, RANKL, SCF, STAT3, STAT4, STAT6, stromal cell-derived factor 1, TALL-1, TBX21, TGF-α, TGF-β, TGF-β1, TGF-β2, TGF-β3, TNF, TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2. In some embodiments, cytokine encoded by the mRNA described herein can include IL-2, IL-12, IL-15, IFN-α, or a combination thereof. In some embodiments, cytokine encoded by the mRNA described herein is IL-2, IL-12, IL-15, and IFN-α.

### Modulators of Tumor Microenvironment

In human cancer, the tumor microenvironment is frequently altered to prevent or suppress anti-tumor immune response. There are various modulators of tumor microenvironment that alter the extracellular matrix to enhance immune cell infiltration or that inflame the milieu to turn cold tumors into hot tumors. Some of these modulators have shown signs of efficacy in the preclinical models. However, some others were not dropped during preclinical or clinical development due to systemic toxicity issues. Present disclosure teach ways, to those skilled in the art, that would allow for the local secretion of such immune modulators, which can enhance intratumoral activity while minimizing systemic effects. In some embodiments, a payload may be a protein such as a kynureninase, adenosine deaminase (ADA2) and 15-hydroxyprostaglandin dehydrogenase (15-PGDH). In some other embodiments, a payload may be an enzyme, such as hyaluronidase and collagenase, which degrades the extracellular matrix and alters the tumor stroma.

### Cell Surface Antigens

Those skilled in the art are aware of various therapeutic technologies that have been developed for treating cancer by immunologically targeting antigens or epitopes expressed on surfaces of tumor cells. In some embodiments, a payload encoded by a translatable nucleic acid for use in accordance with the present disclosure encodes such an antigen or epitope, that may be immunologically targeted by a subject's immune system and/or by immune therapy (e.g., cell therapy such as CAR-T or CAR-NK therapy, or adoptive immunotherapy) administered to the subject. In some embodiments, such a cell surface antigen or epitope may be or comprise an antigen or epitope already expressed by relevant cancer cells; without wishing to be bound by any particular theory, the present disclosure proposes that increased expression of such an antigen or epitope may facilitate its targeting. In some embodiments, such an antigen or epitope may be one not already expressed by relevant tumor cells, in some such embodiments, it may be selected to permit targeting by an existing immune response or therapy.

### Genetic Modification Proteins

Those skilled in the art, reading the present disclosure, will be aware of the relevance of its teachings to genetic modification enzymes and their use, for example, to modify or destroy one or more aspects of a cancer cells' genome, transcriptome, etc.

For example, in some embodiments, a payload encoded by a translatable nucleic acid as described herein may be or comprise a genetic modification protein (e.g., that is or comprises a nuclease). In some embodiments, a genetic modification enzyme may be or comprise a transcription activator-like effector nuclease (TALEN), a zinc finger nuclease (ZFN), one or more components of a CRISPR based gene modification system (e.g., a Cas enzyme).

In some embodiments, a genetic modification protein (e.g., a nuclease) that targets sequences found preferentially or only in relevant cancer cells. However, those skilled in the art, reading the present disclosure, will appreciate that the degree of oncoselectivity in achieves permits use of genetic modification proteins that target sequences that are not particularly specific to cancer cells, as the genetic modification protein itself will be preferentially expressed only in those cells.

### Suicide Proteins

Those skilled in the art will be aware of various proteins commonly referred to as “suicide proteins” (encoded by “suicide genes”) and will appreciate that, in some embodiments, a payload sequence included in a translatable nucleic acid as described herein is or comprises a suicide protein.

In some embodiments, a suicide protein is a protein that induces cell death. In some embodiments, a suicide protein is a protein that induces immunogenic cell death, such as necroptosis, pyroptosis or ferroptosis. The present disclosure provides an insight that certain suicide proteins that induce necroptosis may be particularly advantageous for use in accordance with the present disclosure. For example, the present disclosure observes that necroptosis can induce and/or promote an adaptive immune response. Without wishing to be bound by any particular theory, the present disclosure observes that necroptosis involves immune ligands including Fas, TNF, and LPS leading to activation of RIPK. The present disclosure teaches that use of a necroptotic suicide protein, which may induce and/or promote an adaptive immune response, may facilitate inhibition, destruction and/or removal of tumor cells. In some embodiments, a suicide protein induces apoptosis; in some such embodiments, a suicide protein is p53, or is a protein involved in a p53-mediated apoptosis pathway (e.g. PUMA, BIM, BAX, BAK, tBID, CASPASE-3, CASPASE-7, CASPASE-8, or CASPASE-9).

In some embodiments, a suicide protein is or comprises a protein that renders cells expressing it more susceptible to killing by a separate agent. To give but one example, those skilled in the art are aware of certain viral and/or bacterial enzymes that are not naturally found in mammals and that convert a substance that may be harmless to cells that do not express the enzyme(s) into a toxin. In some embodiments, such a suicide protein is or comprises an enzyme that converts an otherwise inactive agent (e.g., drug) into a toxic antimetabolite, e.g., that inhibits nucleic acid synthesis. In some such embodiments, a suicide protein is a thymidine kinase, wherein the payload sequence encoding thymidine kinase is co-administered with or administered before ganciclovir or valacyclovir treatment.

In some embodiments, a suicide protein payload for use in accordance with the present disclosure is Mixed Lineage Kinase Domain Like Pseudokinase (MLKL), Receptor-interacting serine/threonine-protein kinase 3 (RIPK3), Receptor-interacting serine/threonine-protein kinase 1 (RIPK1), Fas-associated protein with death domain (FADD), or gasdermin D (GSDMD), cysteine-aspartic proteases, cysteine aspartases or cysteine-dependent aspartate-directed proteases (CASPASE-1 or CASP-1), CASPASE-4, CASPASE-5, CASPASE-12, PYCARD/ASC (PYD and CARD domain containing/Fas-associated protein with death domain) or variants thereof.

### Toxins

In some embodiments, a payload for use in accordance with the present disclosure may be or include a toxin protein. Those skilled in the art will be aware of a variety of toxin proteins that may be useful to kill cancer cells. As noted herein, it is one feature of the present disclosure that the degree of oncoselectivity achieved is such that even very potent payloads may be utilized notwithstanding that such payloads might have significant deleterious effects if expressed in non-cancer cells. In some embodiments, a payload is a toxin that is not secreted from a cancer cell.

In some embodiments, a toxin may be or comprise a bacterial toxin. In some embodiments, a toxin may be or comprise a toxin produced by a venomous animal (see, for example, Kozlov et al Rec Pat DNA Gene Sequ 1:200, 2007). In some embodiments, a toxin may be or comprise a plant toxin.

In some embodiments, a toxin that may be utilized as a payload in accordance with the present disclosure may be or comprise a phospholipase or a lecithinase. In some embodiments, a useful toxin may be or comprise a lethal toxin. In some embodiments, a useful toxin may be or comprise an exotoxin. In some embodiments, a useful toxin may be or comprise a pore-forming toxin. In some embodiments, a useful toxin may be or comprise a pyrogenic exotoxin.

In some embodiments, a toxin that may be utilized as a payload is one found in (or derived from) a bacterium that is a bacillus (e.g., Bacillus anthracis), bortadella (e.g., Bortadella pertussis), clostridium (Clostridium botulinum), corynebacterium (e.g., Corynebacterium diphtheriae), eschericia (e.g., Eschericia coli), listeria (e.g., Listeria monocytogenes), pseudomonas (Pseudomonas aeruginosa), staphylococcus (e.g., Staphylocococus aureus), streptococcus, shigella (e.g. Shigella dysenteriae),

In some embodiments, a toxin may be or comprise cholera toxin (e.g., A-5B), diphtheria toxin (e.g., A/B), pertussis toxin (e.g., A-5B), E. coli heat-labile toxin LT (e.g., A-5B), shiga toxin (e.g., A-5B), pseudomonas exotoxin (e.g., A/B), botulinum toxin (e.g., A/B), tetanus toxin (e.g., A/B), anthrax toxin (e.g., lethal factor [LF]), Staphylococcus aureaus exfoliatin B.

In some embodiments, a toxin may be or comprise perfringiolysin (e.g., from Clostridium perfringens), hemolysin (e.g., from Escherichia coli), listeriolysin (e.g., from Listeria monocytogenes), anthrax EF (e.g., from Bacillys anthracis), alpha toxin (e.g., from Staphylococcus aureaus), pneumolysin (e.g., from Streptococcus pneumoniae), streptolysin PO (e.g., from Streptococcus pyogenes), leucocidin (e.g., from Staphylococcus aureus).

In some embodiments, a toxin may be a component of an exotoxin (e.g. Lethal Factor of anthrax toxin), that is, on its own, not capable of being internalized into mammalian cells.

In some embodiments, a toxin may be or comprise ricin or an amanitin. In some embodiments, a toxin may be or comprise alpha-amanitin.

### Inducible or Repressible Proteins

Recent advances in genetic engineering and synthetic biology allow for proteins that are inducible or repressible via small molecule modulators. In some embodiments, a repressible protein can be fused to a Ligand-Induced Degradation (LID) domain, which results in the proteolytic cleavage of the protein upon treatment with the small molecule Shield-1. In some other embodiments, an inducible protein may be inducible Caspase-9, which is activated by the small molecule rimiducid by dimerization. The activated Caspase-9 leads to rapid apoptosis of cells. In some other embodiments, the induction or repression may be achieved via other degradation domains (e.g. dihydrofolate reductase based destabilization domain) or dimerization domains (e.g. FKBP—FRB) and/or other small molecules (e.g. doxycycline, rapamycin, trimethoprim). In some embodiments, a payload for use in accordance with the present disclosure may be or include an inducible or repressible protein.

### Viral Proteins

Those skilled in the art are aware of a variety of viruses that produce proteins useful as payloads as described herein. In some embodiments, a payload may be or comprise a viral protein. In some embodiments, a payload may be LMP1 protein of Epstein-Barr virus. In some embodiments a payload may be or comprise an oncolytic virus protein.

In some embodiments, a payload may be or comprise a viral replication protein. In some embodiments, the viral replication protein is a protein needed for the viral replication cycle. In some embodiments, the viral replication protein is an enzyme. In some embodiments, the viral replication protein is a protease, a polymerase, or a transcriptase.

Those skilled in the art, reading the present disclosure, will appreciate that a variety of technologies are available that can usefully be employed to produce a translatable nucleic acid as described herein. In some embodiments, such production may be ex vivo (i.e., outside of a subject in need of cancer treatment as described herein); in some embodiments, such production may be in vivo.

In some embodiments, a translatable nucleic acid may be produced, wholly or partially, by chemical synthesis and/or chemical modification (e.g., capping)

In some embodiments, a translatable nucleic acid may be produced, wholly or partially, by copying (e.g., via replication or transcription) of a template nucleic acid. In some embodiments, such copying may be ex vivo; in some embodiments, it may be in vivo.

Those skilled in the art, reading the present disclosure, will appreciate that a variety of technologies are available to achieve delivery of a translatable nucleic acid to (at least) cancer cells in accordance with the present disclosure, and furthermore will appreciate that some modes of delivery involve administration of a composition comprising the translatable nucleic acid (e.g., mRNA), and some modes of delivery involve administration of a composition from which the translatable nucleic acid is generated after administration (e.g., via administration of a vector that encodes or templates the translatable nucleic acid.

As noted herein, those skilled in the art will be aware that a variety of administration systems have been developed to achieve effective delivery of nucleic acids into cells, including within mammalian (e.g., human) subjects.

Among such available technologies are various nanoparticle technologies including, for example, hydrogel, lipid, and/or polymer nanoparticle technologies.

In some embodiments, a nucleic acid is delivered to a subject in accordance with the present disclosure using a lipid nanoparticle (LNP). As used herein, the phrase “lipid nanoparticle” refers to a transfer vehicle comprising one or more lipids (e.g., cationic lipids, non-cationic lipids, and PEG-modified lipids). In some embodiments, lipid nanoparticles are formulated to deliver one or more copies of the nucleic acid to one or more target cells.

Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides).

In some embodiments, a nucleic acid is delivered to a subject in accordance with the present disclosure using a polymer nanoparticle. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

In some embodiments, lipids for use in the delivery of a nucleic acid of the present invention include those described in international patent publication WO 2010/053572, incorporated herein by reference. In certain embodiments, the compositions and methods of the invention employ a lipid nanoparticles comprising an ionizable cationic lipid described in U.S. provisional patent application 61/617,468, filed Mar. 29, 2012 (incorporated herein by reference), such as, e.g., (15Z, 18Z)—N,N-dimethyl-6-(9Z, 12Z)-octadeca-9,12-dien-1-yl)tetracosa-15,18-dien-1-amine (HGT5000), (15Z, 18Z)—N,N-dimethyl-6-((9Z, 12Z)-octadeca-9,12-dien-1-yl)tetracosa-4,15,18-trien-1-amine (HGT5001), and (15Z,18Z)—N,N-dimethyl-6-((9Z, 12Z)-octadeca-9, 12-dien-1-yl)tetracosa-5, 15, 18-trien-1-amine (HGT5002).

In some embodiments, the lipid N-[1-(2,3-dioleyloxy)propyl]-N,N,N-trimethylammonium chloride or “DOTMA” is used. (Feigner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolaamine or “DOPE” or other cationic or non-cationic lipids into a liposomal transfer vehicle or a lipid nanoparticle, and such liposomes can be used to enhance the delivery of nucleic acids into target cells. Other suitable lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr et al. Proc. Nat.'l Acad. Sci. 86, 6982 (1989); U.S. Pat. Nos. 5,171,678; 5,334,761), 1,2-Dioleoyl-3-Dimethylammonium-Propane or “DODAP”, 1,2-Dioleoyl-3-Trimethylammonium-Propane or “DOTAP”. Contemplated lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-octadecadienoxy)propane or “CLinDMA”, 2-[5′-(cholest-5-en-3-beta-oxy)-3′-oxapentoxy)-3-dimethyl-1-(cis,cis-9′, l-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-DMA”, 2,2-dilinoleyl-4-dimethylaminoethyl-[1,3]-dioxolane or “DLin-K-XTC2-DMA”, and 2-(2,2-di((9Z,12Z)-octadeca-9,12-dien-1-yl)-1,3-dioxolan-4-yl)-N,N-dimethylethanamine (DLin-KC2-DMA)) (See, WO 2010/042877; Semple et al., Nature Biotech. 28: 172-176 (2010) (Heyes, J., et al., J Controlled Release 107: 276-287 (2005); Morrissey, D V., et al., Nat. Biotechnol. 23(8): 1003-1007 (2005); PCT Publication WO2005/121348A1.), DLin-MC3-DMA (See WO2015199952A1 Tam and Cullis Pharmaceutics. 2013 September; 5(3): 498-507) or mixtures thereof. The use of cholesterol-based cationic lipids is also contemplated by the present invention. Such cholesterol-based cationic lipids can be used, either alone or in combination with other cationic or non-cationic lipids. Suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

In some embodiments, an LNP comprises one or more: (1) “cationic” and/or amino (ionizable) lipids, (2) phospholipids and/or polyunsaturated lipids (helper lipids), (3) structural lipids (e.g., sterols), and/or (4) lipids containing polyethylene glycol (PEG lipids). In some embodiments, an LNP is one as described in WO2021026358.

In some embodiments, an LNP of the present disclosure comprises at least one cationic lipid. In some embodiments, the present disclosure provides LNPs comprising at least one cationic ionizable lipid. In some embodiments the term “cationic ionizable lipid” refers to lipid and lipid-like molecules with nitrogen atoms that can acquire charge (pKa). In some embodiments, a cationic ionizable lipid for use in accordance with the present disclosure has a pKa of 5, 6, 7, 8, 9, 10, 11 at physiological pH. In some embodiments a cationic ionizable lipid comprises one or more groups which is protonated at physiological pH but deprotonates and has no charge at a pH above 5, 6, 7, 8, 9, 10, 11, or 12. In some embodiments a cationic ionizable lipid comprises one or more groups which are protonated and have a charge at a pH above 5, 6, 7, 8, 9, 10, 11, or 12. In some embodiments, the ionizable cationic group may contain one or more protonatable amines which are able to form a cationic group at physiological pH.

One insight provided by the present disclosure is that use of a cationic ionizable lipid with a pKa within a particular range as described herein may be particularly useful for delivery of nucleic acids, for example in LNP preparations as described herein. Specifically, in some embodiments, a cationic ionizable lipid has a high pKA. In some embodiments a high pKa is a pKa greater than 7. In some embodiments a high pKa is a pKa greater than 7.4. In some embodiments, a cationic ionizable lipid for use in accordance with the present disclosure has a pKa of between 7 and 8, 7.5 and 8.5, 8 and 9, 8.5 and 9.5, 9 and 10. In some embodiments, a cationic ionizable lipid for use in accordance with the present disclosure has a pKa of between 7.2 and 8.2, 7.4 and 8.4, 7.6 and 8.6, 7.8 and 8.8, 8.0 to 9.0, 8.2 to 9.2, 8.4 to 9.4, 8.6 to 9.6, 8.8 to 9.8, 9.0 to 10.0. In some embodiments, a useful cationic ionizable lipid has a pKa of 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. This insight is particularly surprising in light of the understanding in the field that pKas>7 are not optimum for LNP payload delivery. See Jayaraman et al., Angew. Chem. Int. Ed. 2012, 51, 8529-8533. Indeed, Jayaraman et al states that LNP potency rapidly decreases if the pKa is outside of the range of 6.2 to 6.5. Further, with respect to intramuscular administration of LNP Hassett et al., Molecular Therapy: Nucleic Acids Vol. 15 Apr. 2019 states that a lipid pKa range of 6.6-6.9 is optimal.

In some embodiments, a cationic ionizable lipid with a high pKA (e.g., >7) is disclosed or described in WO2020219876; US20210162053; US20160317676; and WO2021113365 each of which is incorporated herein in their entirety. In some embodiments, a cationic ionizable lipid with a high pKA (e.g., >7 is one selected from those listed in Table 11.

TABLE 11
Exemplary cationic ionizable lipid with a high pKA

| | |
|---|---|
| 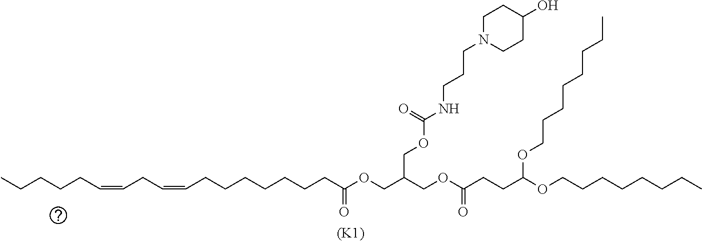 | 3-((4,4-bis(octyloxy) butanoyl)oxy)-2- ((((3-(4- hydroxypiperidin-1- yl)propyl)carbamoyl) oxy)methyl)propyl (9Z, 12Z)-octadeca- 9, 12-dienoate |
| 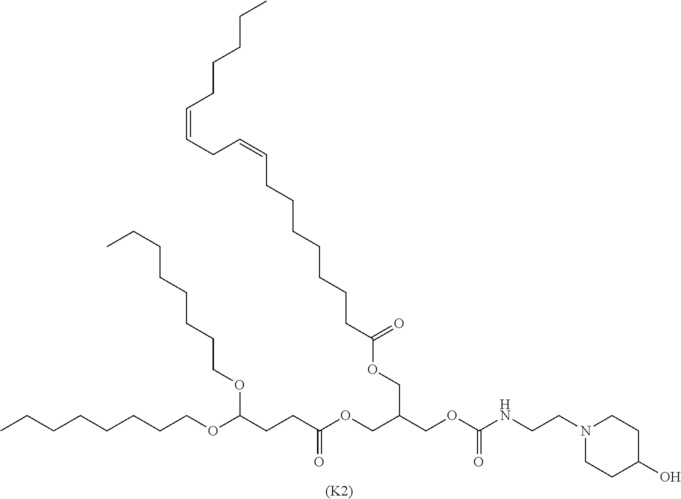 | 3-((4,4-bis(octyloxy) butanoyl)oxy)-2- ((((2-(4- hydroxypiperidin-1- yl)ethyl)carbamoyl) oxy)methyl)propyl (9Z, 12Z)-octadeca- 9, 12-dienoate |
| 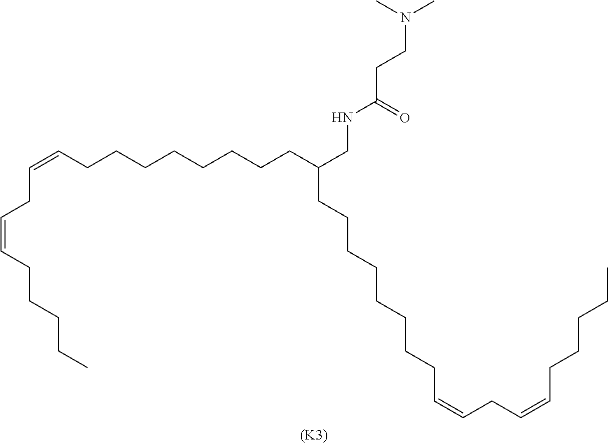 [SMILES: CCCCC/C=C\C/C=C\CCCCCCCCC(CCCCCCCC/C=C\C/C=C\CCCCC)CNC(=O)CCN(C)C ; "(K3)"] | 3-(dimethylamino)- N-((11Z,14Z)-2- ((9Z,12Z)-octadeca- 9,12-dienyl)icosa- 11,14-dienyl) propanamide |
| 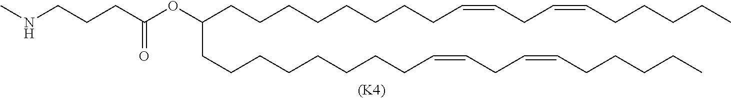 | DLin-M-C3-EA |
| 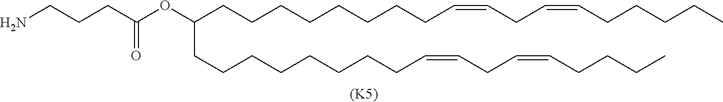 | DLin-M-C3-A |
| 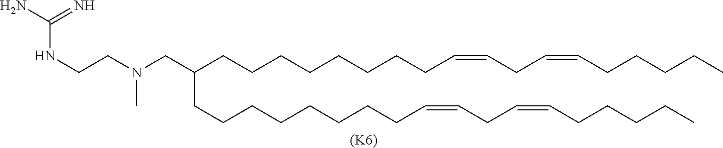 | |
| 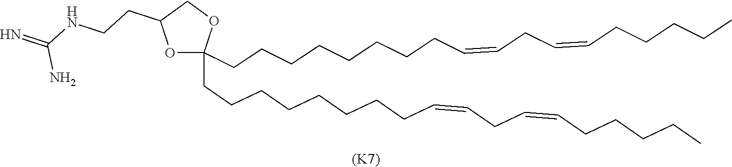 | |
| 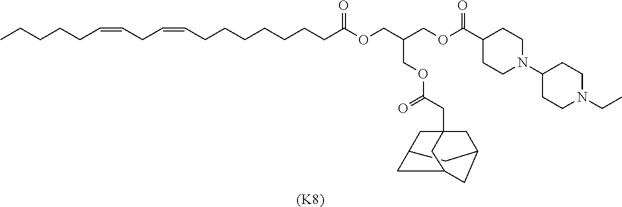 [SMILES: CCCCC/C=C\C/C=C\CCCCCCCC(=O)OCC(COC(=O)CC12CC3CC(CC(C3)C1)C2)COC(=O)C1CCN(C2CCN(CC)CC2)CC1 ; "(K8)"] | |
| 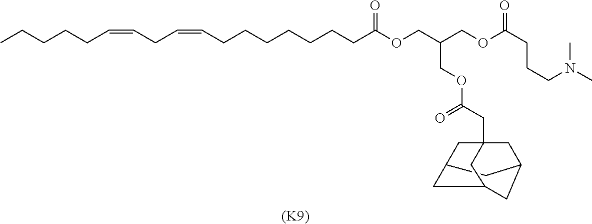 [SMILES: CCCCC/C=C\C/C=C\CCCCCCCC(=O)OCC(COC(=O)CCCN(C)C)COC(=O)CC12CC3CC(CC(C3)C1)C2 ; "(K9)"] | |
| 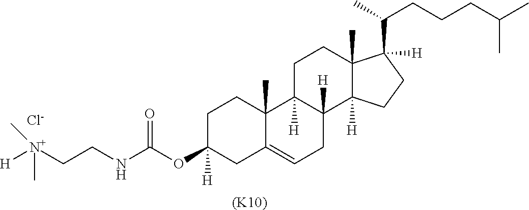 | DC-Cholesterol |

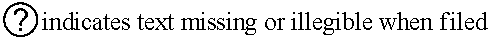

Among other things, the present disclosure demonstrates that use of a high pKa cationically ionizable lipid in LNPs as described herein may be particularly useful to achieve delivery of nucleic acids to the lung.

In some embodiments the present disclosure provides LNPs comprising a at least first cationic ionizable lipid and a second cationic ionizable lipid. In some such embodiments, more than one (e.g., each) of such at least first and second cationic ionizable lipids has a high pKa (e.g., greater than 7, e.g., greater than 7.4) as described herein. Alternatively, in some such embodiments, only one (i.e., a first) cationic ionizable lipid has such a high pKa (e.g., greater than 7, e.g., greater than 7.4) as described herein. In some embodiments, a second cationic ionizable lipid is a non-high-pKa lipid, e.g., in that it has a pKa below 7, e.g., about 6.8, 6.6, 6.4, 6.2, 6.0, 5.8, 5.6, 5.4, 5.2, 5.0 or lower. In some embodiments, a non-high-pKa lipid (e.g., a second lipid) has a pKa of about 6.4 or lower. In some embodiments, a non-high-pKa lipid (e.g., a second lipid) has a pKa of about 6.4. In some embodiments, a non-high-pKa lipid (e.g., a second lipid) has a neutral pKa.

In some embodiments, a cationic ionizable lipid with a non-high pKa (e.g., <7) is disclosed or described in Finn et al., 2018 Cell Reports 22, 2227-2235; Jayaraman et al., Angew. Chem. Int. Ed. 2012, 51, 8529-8533; Hassett et al., Molecular Therapy: Nucleic Acids Vol. 15 Apr. 2019; WO2015074085; WO2020118041; WO2020072605; WO2020252589; WO2021055849; WO2018232120; WO2021030701; WO2020146805; WO2019036000; WO2018200943; and WO2018191657 each of which is incorporated herein in their entirety. In some embodiments, a cationic ionizable lipid with a non-high pKA (e.g., <7) is one selected from those listed in Table 12.

TABLE 12
Exemplary cationic ionizable lipid with a non-high pKA

| | |
|---|---|
| 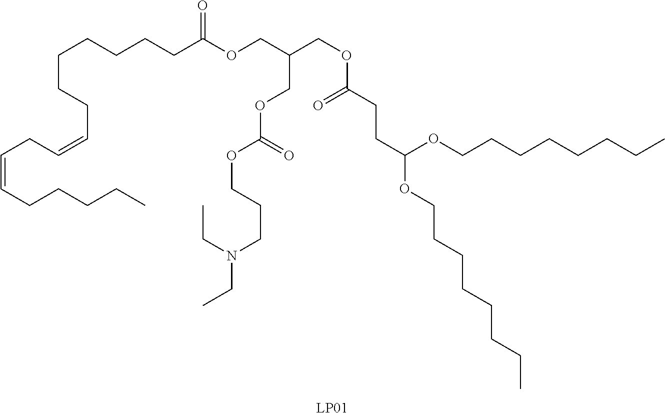 [SMILES: CCCCC/C=C\C/C=C\CCCCCCCC(=O)OCC(COC(=O)CCC(OCCCCCCCC)OCCCCCCCC)COC(=O)OCCCN(CC)CC] | (K11) |
| 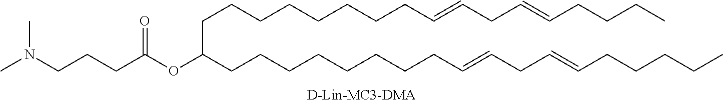 [SMILES: CCCC/C=C/C/C=C/CCCCCCCCC(CCCCCCCC/C=C/C/C=C/CCCCC)OC(=O)CCCN(C)C] | (K12) |
| 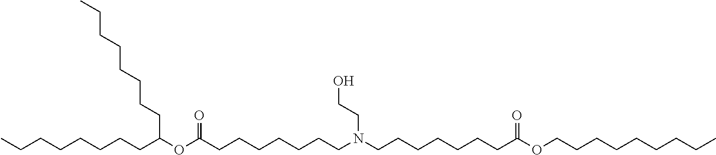 [SMILES: CCCCCCCCCOC(=O)CCCCCCCN(CCO)CCCCCCCC(=O)OC(CCCCCCCC)CCCCCCCC] | (K13) |
| 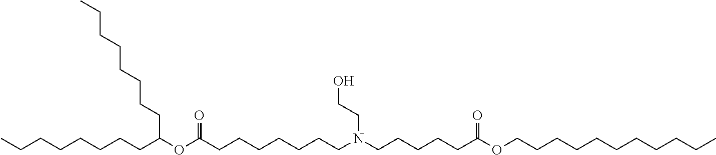 [SMILES: CCCCCCCCCCCOC(=O)CCCCCN(CCO)CCCCCCCC(=O)OC(CCCCCCCC)CCCCCCCC] | (K14) |
| 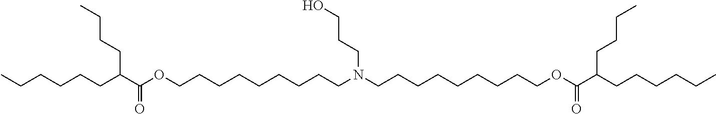 [SMILES: CCCCCCC(CCCC)C(=O)OCCCCCCCCCN(CCCO)CCCCCCCCCOC(=O)C(CCCC)CCCCCC] | (K15) |
| 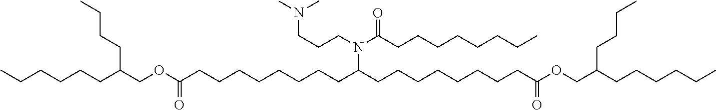 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] | (K16) |
| 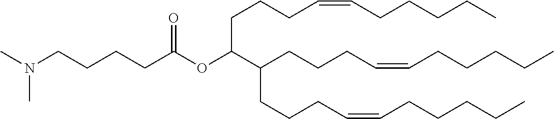 [SMILES: CCCCC/C=C\CCCC(CCC/C=C\CCCCC)C(CCC/C=C\CCCCC)OC(=O)CCCCN(C)C] | (K17) |
| 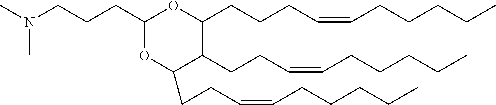 [SMILES: CCCCC/C=C\CCCC1OC(CCCN(C)C)OC(CC/C=C\CCCCC)C1CC/C=C\CCCCC] | (K18) |

In some embodiments, an LNP comprises about 0-80 mol % of high pKa cationic ionizable lipid as described herein; in some embodiments, an LNP comprises two or more high pKa cationic ionizable lipids that, together make up such 0-80 mol % of the LNP. In some embodiments, an LNP comprises about 20-80 mol % of high pKa cationic ionizable lipid. In some embodiments, an LNP comprises about 0, 5, 10, 15, 20, 25, 30, 35, 40, 45, 50, 55, 60, 65, 7-, 75, 80 mol % of high pKa cationic ionizable lipid.

In some embodiments, an LNP comprises about 0-60 mol % of cationic ionizable lipid that is not high pKa (e.g., that is characterized by a pKa below about 7, such as a pKa of about 6.4 or a neutral pKa); in some embodiments, an LNP comprises two or more non-high pKa cationic ionizable lipids that, together, make up such 0-60 mol % of the LNP. In some embodiments, an LNP comprises about 0, 5, 10, 15, 20, 25, 30, 35, 40, 45, 50, 55, 60 mol % of a non-high-pKa cationic ionizable lipid.

In some embodiments, the present disclosure provides LNPs comprising at least one sterol. In some embodiments, an LNP comprises about 0-40 mol % of a sterol. In some embodiments, an LNP comprises about 0, 5, 10, 15, 20, 25, 30, 35, 40 mol % of a sterol.

In some embodiments, a sterol is cholesterol, or a variant or derivative thereof. In some embodiments, a cholesterol is modified, for example oxidized. Unmodified cholesterol can be acted upon by enzymes to form variants that are side-chain or ring oxidized. In some embodiments, a cholesterol can be oxidized on the beta-ring structure or on the hydrocarbon tail structure. Exemplary cholesterols that are considered for use in the disclosed LNPs include but are not limited to 25-hydroxycholesterol (25-OH), 20α-hydroxycholesterol (20α-OH), 27-hydroxycholesterol, 6-keto-5α-hydroxycholesterol, 7-ketocholesterol, 7β-hydroxycholesterol, 7α-hydroxycholesterol, 7β-25-dihydroxycholesterol,beta-sitosterol, stigmasterol, brassicasterol, campesterol, or combinations thereof. In some embodiments, side-chain oxidized cholesterol can enhance cargo delivery relative to other cholesterol variants. In some embodiments, a cholesterol is an unmodified cholesterol.

In some embodiments, the present disclosure provides LNPs comprising at least one helper lipid. In some embodiments a helper lipid is a phospholipid. In some embodiments, an LNP comprises about 0-20 mol % of a helper lipid. In some embodiments, an LNP comprises about 0, 5, 10, 15, 20 mol % of a helper lipid.

Exemplary phospholipids include but are not limited to 1,2-distearoyl-snglycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycerophosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycerophosphocholine (DUPC), 1-palmitoyl-2-oleoyl-sn-glycero-3-phosphocholine (POPC), 1,2-di-0-octadecenyl-sn-glycero-3-phosphocholine (18:0 Diether PC), 1-oleoyl-2-cholesterylhemisuccinoy 1-sn-glycero-3-phosphocholine (OChemsPC), 1-hexadecyl snglycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2 oleoylphosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or combinations thereof. In some embodiments, a phospholipid is DSPC. In some embodiments, a phospholipid is DMPC.

In some embodiments, a phospholipid comprises 1,2-dioleoyl-sn-glycero-3-phosphoethanolamine-N-(succinyl) (succinyl PE), 1,2-distearoyl-sn-glycero-3-phosphocholine (DSPC), cholesterol, 1,2-distearoyl-sn-glycero-3-phosphoethanolamine (DSPE), 1,2-dipalmitoyl-sn-glycero-3-phosphoethanolamine-N-(succinyl) (succinyl-DPPE), 1,2-dioleoyl-sn-glycero-3-phosphoethanolamine (DOPE), 1,2-dimyristoyl-sn-glycero-3-phosphocholine (DMPC), 1,2-dipalmitoyl-sn-glycero-3-phosphocholine (DPPC), or a combination thereof.

In some embodiments, the present disclosure provides LNPs comprising at least one PEGylated lipid. In some embodiments, an LNP comprises about 0-25 mol % of a PEGylated lipid. In some embodiments, an LNP comprises about 0, 5, 10, 15, 20, 25 mol % of a PEGylated lipid.

In some embodiments, inclusion of a PEGylated lipid can be used to enhance lipid nanoparticle colloidal stability in vitro and circulation time in vivo. In some embodiments, PEGylation is reversible in that the PEG moiety is gradually released in blood circulation. Exemplary PEGylated-lipids include but are not limited to PEG conjugated to saturated or unsaturated alkyl chains having a length of C6-C20, PEG-modified-28-phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof. For example, a PEGylated-lipid may be PEG-c-DOMG, PEG-DMG, PEG-DLPE, PEG-DMPE, PEG-DPPE, PEG-DSG or a PEGDSPE lipid.

In some embodiments, the present disclosure provides LNPs comprising at least one PEG-ligand. In some embodiments, an LNP comprises about 0-2 mol % of a PEG-ligand. In some embodiments, an LNP comprises about 0, 0.01, 0.05, 0.1, 0.5, 0.75, 1, 1.5, 1.75, 2 mol % of a PEG-ligand. In some embodiments, a ligand (e.g., as included in a PEG-ligand) is or comprises: antibodies targeting cell surface proteins, hyaluronic acid, small molecules, peptides, and/or peptides that target integrins.

In some embodiments, a translatable nucleic acid as described herein may be delivered to a subject by administration of a nucleic acid vector that encodes and/or templates the translatable nucleic acids. In some embodiments, a useful vector may be or comprise a viral vector.

In some embodiments, a vector system (e.g., a viral vector system) may be or comprise components and/or sequences found in nature (i.e., wild type components and/or sequences); in some embodiments, a vector system may be or comprise engineered components and/or sequences (i.e., components whose sequence has been modified relative to an appropriate wild type reference and/or components that are not found together in a wild type reference but may, for example, represent an assemblage of components from a plurality of different sources).

Those skilled in the art are familiar with a variety of viral vector systems that could be useful in accordance with the present disclosure.

In some embodiments, a viral vector system may be or comprise components of a virus that preferentially infects cancer cells (e.g., an oncolytic virus). Those skilled in the art are aware of a variety of oncolytic viruses including, for example, vaccinia virus, a vesicular stomatitis virus, a poliovirus, a reovirus, a senecavirus, and adenovirus.

The present disclosure provides an insight that use of an oncolytic viral vector system may have certain advantages, for example in potentially providing a complementary mechanism of killing for tumor cells.

However, as noted herein, the degree of oncoselectivity achieved in accordance with the present disclosure renders oncoselectivity of a nucleic acid delivery vector not critical to many embodiments of the disclosure.

As described herein, the present disclosure provides technologies that are particularly useful in the treatment of cancer.

In some embodiments, provided technologies are applied to subjects suffering from cancer. That is, in some embodiments, a translatable nucleic acid as described herein (e.g., comprising at least one oncoselective translation sequence elements and a payload-encoding sequence) is delivered to (e.g., by administration of a composition comprising the translatable nucleic acid, or of a composition that causes the translatable nucleic acid to be generated in or by the subject).

In some embodiments, a subject has received, is receiving and/or will receive other therapy (e.g., other therapy to treat the cancer and/or one or more side effects of the cancer or its treatment). In some such embodiments, a payload is or comprises a protein that increases susceptibility of cells to the other therapy.

In some embodiments, a subject is not receiving a pharmaceutical agent that is known to cause stop codon readthrough in healthy cells. In some embodiments, a subject is not receiving aminoglycosides and/or macrolides.

In some embodiments, a subject is not receiving cystic fibrosis and/or Duchenne muscular dystrophy therapy (e.g. Ataluren or PTC124).

In some embodiments, a subject is not receiving pyronaridine tetraphosphate (anti-malarial), and potassium para-aminobenzoate (PABA, used of Peyronie's disease), experimental compounds RTC13, RTC14, and NB54, and/or herbal supplement escin.

In some embodiments, a subject is not affected by ribosomopathies such as Diamond-Blackfan anemia, Dyskeratosis congenita, Shwachman-Diamond syndrome, 5q-myelodysplastic syndrome, Treacher Collins syndrome, Cartilage-hair hypoplasia, Isolated congenital asplenia, Bowen-Conradi syndrome, North American Indian childhood cirrhosis.

### Resistant or Refractory to Cancer Therapy

In some embodiments, provided technologies are applied to subjects suffering from cancer that have received or are receiving treatment. In some embodiments, a subject has received or is receiving immune checkpoint inhibitor therapy. In some embodiments, a subject are resistant or refractory to immune checkpoint therapy they have received. In some embodiments, provided technologies are applied to subjects based on diagnosis of cancer refractory or to immune checkpoint therapy. In some embodiments, a subject has demonstrated and/or been diagnosed with immune checkpoint therapy relapse (e.g. a non-beneficial response to immune checkpoint therapy; or progressive disease). In some embodiments, biomarkers or indicators of a cancer resistant or refractory to immune checkpoint therapy are described.

As described herein, the present disclosure provides technologies that are particularly useful in the treatment of cancer.

In some embodiments, provided technologies are applied to subjects suffering from cancer. That is, in some embodiments, a translatable nucleic acid as described herein (e.g., comprising at least one oncoselective translation sequence elements and a payload-encoding sequence) is delivered to (e.g., by administration of a composition comprising the translatable nucleic acid, or of a composition that causes the translatable nucleic acid to be generated in or by the subject).

In some embodiments, a subject has received, is receiving and/or will receive other therapy (e.g., other therapy to treat the cancer and/or one or more side effects of the cancer or its treatment). In some such embodiments, a payload is or comprises a protein that increases susceptibility of cells to the other therapy.

In some embodiments, a subject is not receiving a pharmaceutical agent that is known to cause stop codon readthrough in healthy cells. In some embodiments, a subject is not receiving aminoglycosides and/or macrolides.

In some embodiments, a subject is not receiving cystic fibrosis and/or Duchenne muscular dystrophy therapy (e.g. Ataluren or PTC124).

In some embodiments, a subject is not receiving pyronaridine tetraphosphate (anti-malarial), and potassium para-aminobenzoate (PABA, used of Peyronie's disease), experimental compounds RTC13, RTC14, and NB54, and/or herbal supplement escin.

In some embodiments, a subject is not affected by ribosomopathies such as Diamond-Blackfan anemia, Dyskeratosis congenita, Shwachman-Diamond syndrome, 5q-myelodysplastic syndrome, Treacher Collins syndrome, Cartilage-hair hypoplasia, Isolated congenital asplenia, Bowen-Conradi syndrome, North American Indian childhood cirrhosis.

In some embodiments, provided technologies are applied to subjects suffering from cancer that have received or are receiving treatment. In some embodiments, a subject has received or is receiving immune checkpoint inhibitor therapy. In some embodiments, a subject are resistant or refractory to immune checkpoint therapy they have received. In some embodiments, provided technologies are applied to subjects based on diagnosis of cancer refractory or to immune checkpoint therapy. In some embodiments, a subject has demonstrated and/or been diagnosed with immune checkpoint therapy relapse (e.g. a non-beneficial response to immune checkpoint therapy; or progressive disease). In some embodiments, biomarkers or indicators of a cancer resistant or refractory to immune checkpoint therapy are described.

### Method

### Delivery

The engineered polynucleotide can be readily introduced into a cell, e.g., mammalian, bacterial, yeast, or insect cell by any method in the art. For example, the engineered polynucleotide can be transferred into a host cell by physical, chemical, or biological means. In some embodiments, the engineered polynucleotide can be delivered into the cell via physical methods such as calcium phosphate precipitation, lipofection, particle bombardment, microinjection, gene gun, electroporation, and the like.

Physical methods for introducing the engineered polynucleotide encoding into the cell can include calcium phosphate precipitation, lipofection, particle bombardment, microinjection, gene gun, electroporation, and the like. One method for the introduction of the engineered polynucleotide a host cell is calcium phosphate transfection.

Chemical means for introducing the engineered polynucleotide encoding the non-naturally into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An example colloidal system for use as a delivery vehicle in vitro and in vivo is a liposome (e.g., an artificial membrane vesicle). Other methods of state-of-the-art targeted delivery of nucleic acids are available, such as delivery of engineered polynucleotide or vector encoding the engineered polynucleotide with targeted nanoparticles.

In the case where a non-viral delivery system is utilized, an example delivery vehicle is a liposome. The use of lipid formulations is contemplated for the introduction of the engineered polynucleotide or vector encoding the engineered polynucleotide into a cell (in vitro, ex vivo, or in vivo). In another aspect, the vector can be associated with a lipid. The vector associated with a lipid can be encapsulated in the aqueous interior of a liposome, interspersed within the lipid bilayer of a liposome, attached to a liposome via a linking molecule that is associated with both the liposome and the engineered polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid:cargo conjugates (or aggregates), naked polypeptide (e.g., recombinant polypeptides), naked DNA, artificial virions, and agent-enhanced uptake of polypeptide or DNA. In some embodiments, the delivery method comprises conjugating or encapsulating the compositions or the engineered polynucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the engineered polynucleotide described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some embodiments, the engineered polynucleotide can be delivered into the cell via biological methods such as the use of DNA and RNA vectors. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAV vectors), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome.

In some instances, the viral vector is a chimeric viral vector, comprising viral portions from two or more viruses. In additional instances, the viral vector is a recombinant viral vector. In some cases, the vector comprises additional features. Additional features can comprise sequences such as tags, signaling peptides, intronic sequences, promoters, stuffer sequences, and the like. In some cases, the vector comprises a signaling peptide. A signaling peptide is sometimes referred to as signaling sequence, targeting signal, localization signal, localization sequence, transit peptide, leader sequence or leader peptide, is a short peptide present at the N-terminus of the majority of newly synthesized proteins that are destined toward the secretory pathway. These proteins include those that reside either inside certain organelles (the endoplasmic reticulum, Golgi or endosomes), secreted from the cell, or inserted into most cellular membranes. In some cases, nucleic acids provided herein can comprise signaling peptides. A signaling peptide can be of any length but typically from 15-30 amino acids long. A signaling peptide can be from about: 10-15, 10-20, 10-30, 15-20, 15-25, 15-30, 20-30, or 25-30 amino acids long. Various signaling peptides can be utilized and include but are not limited to: human antibody heavy chain (Vh), human antibody light chain (Vl), and aflibercept.

In an embodiment, an additional feature of the vector includes promoter. Promoter is sequences of DNA to which proteins bind that initiate transcription of a single RNA from the DNA downstream of it. This RNA may encode a protein, or can have a function in and of itself, such as tRNA, mRNA, or rRNA. Promoters are located near the transcription start sites of genes, upstream on the DNA (towards the 5′ region of the sense strand). Promoters can be about 100-1000 base pairs long. In some cases, the promoters can be inducible promoters. Various promoters are contemplated and can be employed in the vectors of the disclosure. In an embodiment, a promoter is: a cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, a Ca2+/calmodulin-dependent protein kinase II (CaMKIIa) promoter, a GAL1 promoter, a GAL 10 promoter, a TEF1 promoter, a glyceraldehyde 3-phosphage dehydrogenase (GDS) promoter, an ADH1 promoter, a CaMV35S promoter, a Ubi promoter, a human polymerase III RNA (H1) promoter, a U6 promoter, a polyadenylated construct thereof, and any combination thereof. In some cases, the promoter is the CMV promoter.

In some embodiments, the vector comprising the at least two expression cassettes under expression control of two different promoters. Such arrangement allows the two signaling transduction regulators to be expressed simultaneously or in a desired sequential order in a cell.

### Treatment

Provided herein are methods of treating a disease or condition described here. A method of treatment can comprise introducing to a subject in need a engineered polynucleotide. Also provided is a method of treating disease or condition that comprises administering a pharmaceutical composition to a subject in need thereof. A pharmaceutical composition can comprise a sequence that encodes a biologic that comprises the engineered polynucleotide. In some embodiments, administration is by any suitable mode of administration, including systemic administration (e.g., intravenous, inhalation, vitreous, or etc.). In some embodiments, the subject is human.

In some embodiments, the engineered polynucleotide is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, or 100 times) during a period of time.

In some embodiments, the method comprises administering the engineered polynucleotide in an engineered therapeutically-effective amount by various forms and routes including, for example, intratumoral, oral, or topical administration. In some embodiments, a composition may be administered by intratumoral, parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the composition is delivered via multiple administration routes.

Actual dosage levels of an agent of the disclosure (e.g., the engineered polynucleotide or a pharmaceutical composition) may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., an engineered therapeutic and/or prophylactic response). For example, a single bolus may be administered, several divided doses may be administered over time or the dose may be proportionally reduced or increased as indicated by the exigencies of the engineered therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

The engineered polynucleotide, the vector comprising the engineered polynucleotide, or the pharmaceutical composition described herein may be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

Described herein, in some aspects, is a method for treating a disease or condition in a subject, comprising administering to the subject an engineered polynucleotide, said engineered polynucleotide comprises at least one oncoselective sequence; and a coding sequence encoding an engineered therapeutic, wherein the at least one oncoselective sequence comprises a nucleic acid sequence that is: at least 75% identical to any one SEQ ID NOs: 1-97 or SEQ ID NOs: 101-146. In some embodiments, the at least one oncoselective sequence comprises the nucleic acid sequence that is at least 80%, at least 85%, at least 90%, at least 95%, or at least 99% identical to any one of SEQ ID NOs: 1-97 or SEQ ID NOs: 101-146. In some embodiments, the at least one oncoselective sequence comprises the nucleic acid sequence that is any one of SEQ ID NOs: 1-97 or SEQ ID NOs: 101-146. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-75, 91-97, or 101-146. In some embodiments, the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-75, 91-97, or 101-146. In some embodiments, the at least one oncoselective sequence comprises the nucleic acid sequence that is at least 5 contiguous nucleotides identical to any one of SEQ ID NOs: 76-90.

In some embodiments, the engineered polynucleotide comprises decreased immunogenicity. In some embodiments, the at least one oncoselective sequence increases expression of the engineered therapeutic in a cancer cell compared to expression of the engineered therapeutic in a normal cell. In some embodiments, the at least one oncoselective sequence increases expression of the engineered therapeutic in a cancer cell compared to expression of the engineered therapeutic in a non-cancerous cell. In some embodiments, the at least one oncoselective sequence does not increase expression of the engineered therapeutic in a liver cell. In some embodiments, the at least one oncoselective sequence does not increase expression of the engineered therapeutic in a liver cell.

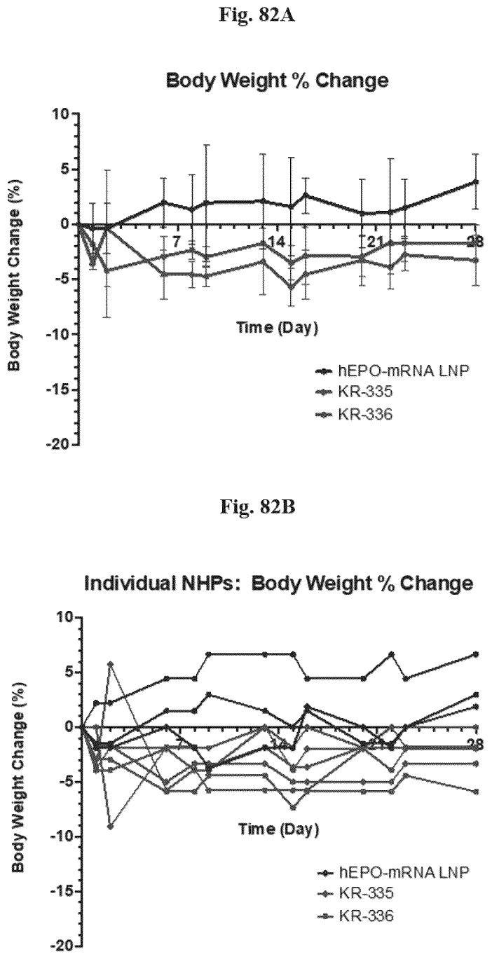

In some embodiments, described herein is a method for treating a disease or condition in a subject, comprising administering to the subject an engineered polynucleotide, said engineered polynucleotide comprises: at least one oncoselective sequence; and a coding sequence encoding an engineered therapeutic. In some embodiments, engineered therapeutic comprises: an engineered interleukin or fragment thereof described herein or an engineered subunit of an interleukin described herein. In some embodiments, the engineered therapeutic comprises at least one cytokine or at least one interferon. In some embodiments, the engineered therapeutic can be part of combination for a therapeutic cocktail. As shown in FIGS. 36-50, the specific combination of an engineered therapeutic cocktail (IL-2, IL-12, IL-15, and IFN-α) increases therapeutic efficacies of treating tumors and cancer while not increasing adverse side effects. In some embodiments, the any one of the cocktail combination (e.g., IL-2, IL-12, IL-15, or IFN-α) can be substituted with an engineered cytokine described herein. For example, cocktail composition can include an engineered IL-2 or engineered IL-12 or both in place of IL-2 or IL-12. Such substitution with the engineered cytokine can further increase (e.g., synergistically) the engineered therapeutic efficacies of treatment by the combination. In some embodiments, the use of the combination of the engineered therapeutics comprising cytokine or interferon does not increase adverse event. For example, Example 11 and FIGS. 82-87 illustrate lack of adverse event (AE) stemmed from treatment of the combination of the therapeutic described herein.

In some embodiments, the engineered therapeutic is encoded from a nucleic acid sequence that is at least 75%, at least 80%, at least 85%, at least 90%, at least 95%, or at least 99% identical to any one of SEQ ID NOs: 461-476. In some embodiments, the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 351-361, 371-373, 481-490, 501-510, or 601-685. In some embodiments, the engineered therapeutic comprises an engineered cytokine comprising a secreted cytokine, a membrane tethered cytokine, a masked cytokine, a cytokine fusion, or a combination thereof. In some embodiments, the cytokine comprises an interleukin or an interferon. In some embodiments, the interleukin comprises an IL-2. In some embodiments, the IL-2 is encoded from a nucleic acid sequence that is at least 75%, at least 80%, at least 85%, at least 90%, at least 95%, or at least 99% identical to any one of SEQ ID NOs: 467 or 468. In some embodiments, the IL-2 comprises an amino acid sequence that is at least 75%, at least 80%, at least 85%, at least 90%, at least 95%, or at least 99% identical to any one of SEQ ID NOs: 351-361, 371-373, 481, 482, 501, 502, 614-621, or 664-667. In some embodiments, the interleukin comprises an IL-12. In some embodiments, the IL-12 is encoded from a nucleic acid sequence that is at least 75%, at least 80%, at least 85%, at least 90%, at least 95%, or at least 99% identical to any one of SEQ ID NOs: 461, 462, 475, or 476. In some embodiments, the IL-12 comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, 503-507, or 648-663. In some embodiments, the interleukin comprises an IL-15. In some embodiments, the IL-15 is encoded from a nucleic acid sequence that is at least 75%, at least 80%, at least 85%, at least 90%, at least 95%, or at least 99% identical to any one of SEQ ID NOs: 463, 464, 469, or 470. In some embodiments, the IL-15 comprises an amino acid sequence that is at least 75%, at least 80%, at least 85%, at least 90%, at least 95%, or at least 99% identical to SEQ ID NOs: 488 489, 508, 509, 622-647, 668, or 669. In some embodiments, the cytokine comprises the interferon. In some embodiments, the interferon comprises an IFN-α. In some embodiments, the IFN-α is encoded from a nucleic acid sequence that is at least 75%, at least 80%, at least 85%, at least 90%, at least 95%, or at least 99% identical to any one of SEQ ID NOs: 465 or 466. In some embodiments, the IFN-α comprises an amino acid sequence that is at least 75%, at least 80%, at least 85%, at least 90%, at least 95%, or at least 99% identical to SEQ ID NOs: 490, 510, 601-613, 674, 678, or 685.

In some embodiments, the engineered interleukin or fragment thereof comprises an engineered 11-12 or fragment thereof or an engineered subunit of IL-12. In some embodiments, the engineered interleukin or fragment thereof comprises an engineered IL-2 or fragment thereof. In some embodiments, the engineered therapeutic comprises a masking domain, a half-life extension domain, a linker, a signal peptide, or a combination thereof. In some embodiments, the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, 501-510, or 648-663. In some embodiments, the engineered therapeutic comprises an amino acid sequence that is any one of SEQ ID NOs: 301-303, 311-314, 321-328, 331-335, 341-344, 483-487, 501-510, or 648-663. In some embodiments, the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 301-303, 311-314, 321-328, 331-335, 341-344, 483-487, 501-510, or 648-663.

In some embodiments, the engineered therapeutic is encoded by the engineered polynucleotide comprising a nucleic acid sequence that is at least 75%, at least 80%, at least 85%, at least 90%, at least 95%, or at least 99% identical to any one of SEQ ID NOs: 461-476. In some embodiments, the engineered therapeutic is encoded by the engineered polynucleotide comprising a nucleic acid sequence that is any one of SEQ ID NOs: 461-476. In some embodiments, the engineered therapeutic is encoded by the engineered polynucleotide comprising a nucleic acid sequence that is at least 100 contiguous nucleotides, at least 150 contiguous nucleotides, at least 200 contiguous nucleotides, at least 250 contiguous nucleotides, at least 300 contiguous nucleotides, at least 350 contiguous nucleotides, at least 400 contiguous nucleotides, at least 450 contiguous nucleotides, at least 500 contiguous nucleotides, at least 550 contiguous nucleotides, or at least 600 contiguous nucleotides to any one of SEQ ID NOs: 461-476. In some embodiments, the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 351-361, 371-373, 461-476, 481-490, 501-510, or 601-685. In some embodiments, the engineered therapeutic comprises an amino acid sequence that is at any one of SEQ ID NOs: 301-303, 311-314, 321-328, 331-335, 341-344, 351-361, 371-373, 481-490, 501-510, or 601-685. In some embodiments, the engineered therapeutics comprises an amino acid sequence that is at least 50 contiguous amino acids, at least 100 contiguous amino acids, or at least 150 contiguous amino acids identical to any one of 301-303, 311-314, 321-328, 331-335, 341-344, 351-361, 371-373, 481-490, 501-510, or 601-685.

In some embodiments, the at least one oncoselective sequence comprises a nucleic acid motif. In some embodiments, the nucleic acid motif comprises an oncoselective readthrough motif. In some embodiments, the at least one oncoselective sequence comprises an untranslated region (UTR). In some embodiments, the at least one oncoselective sequence comprises a nucleic acid sequence that is at least 75%, at least 80%, at least 85%, at least 90%, at least 95%, or at least 99% identical to any one SEQ ID NOs: 1-97 or 101-146. In some embodiments, the at least one oncoselective sequence comprises a nucleic acid sequence that is at least 50 contiguous nucleotides, at least 100 contiguous nucleotides, or at least 150 contiguous nucleotides identical to any one of SEQ ID NOs: 1-75, 91-97, or 101-146; or at least 5 contiguous nucleotides identical to any one of SEQ ID NOs: 76-90.

In some embodiments, the engineered therapeutic comprises a cytokine such as an engineered cytokine comprising a secreted cytokine, a membrane tethered cytokine, a masked cytokine, a cytokine fusion, or a combination thereof. In some embodiments, the cytokine comprises an interleukin or an interferon. In some embodiments, the cytokine comprises an IL-2, an IL-12, an IL-15, an IFN-α, or a combination thereof. In some embodiments, the disease or condition comprises solid tumor. In some embodiments, the disease or condition comprises cancer. In some embodiments, the cancer comprises melanoma, breast cancer, basal cell carcinoma (BCC), squamous cell carcinoma (SCC), cutaneous SCC (cSCC or CSCC), Head & Neck Cancer, Thyroid Cancer, Colorectal Cancer, Prostate Cancer, Liver cancer, Pancreatic Cancer, Renal Cell Carcinoma, Brain Cancer, Soft Tissue Sarcoma, Lung Cancer, or a combination thereof. In some embodiments, the cancer does not respond to immune checkpoint inhibitor treatment. In some embodiments, the at least one oncoselective sequence increases expression of the engineered therapeutic in a cancer cell compared to expression of the engineered therapeutic in a normal cell.

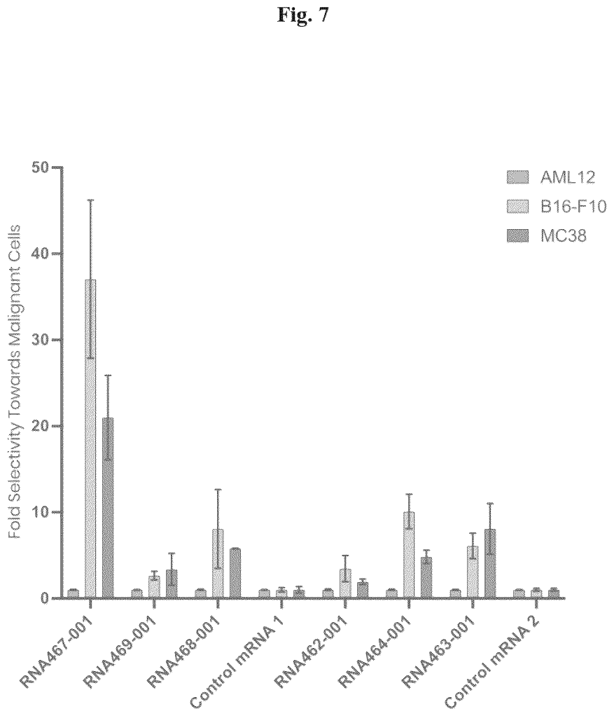

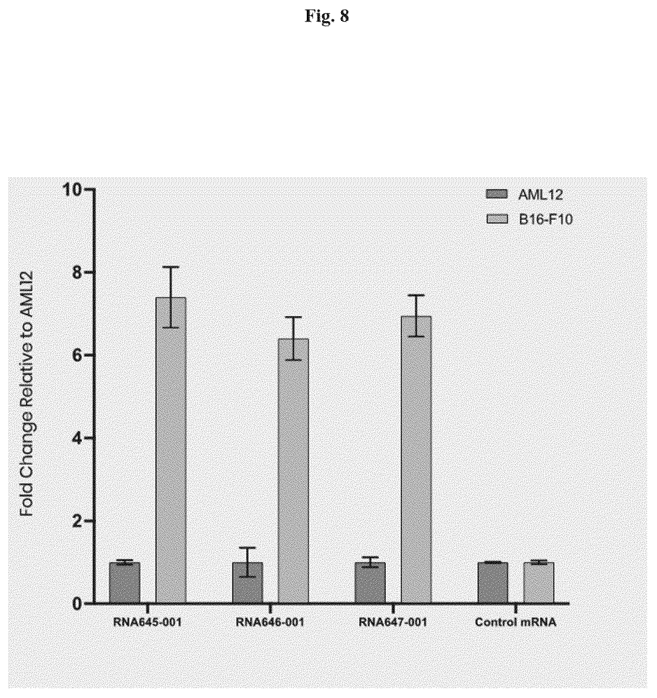

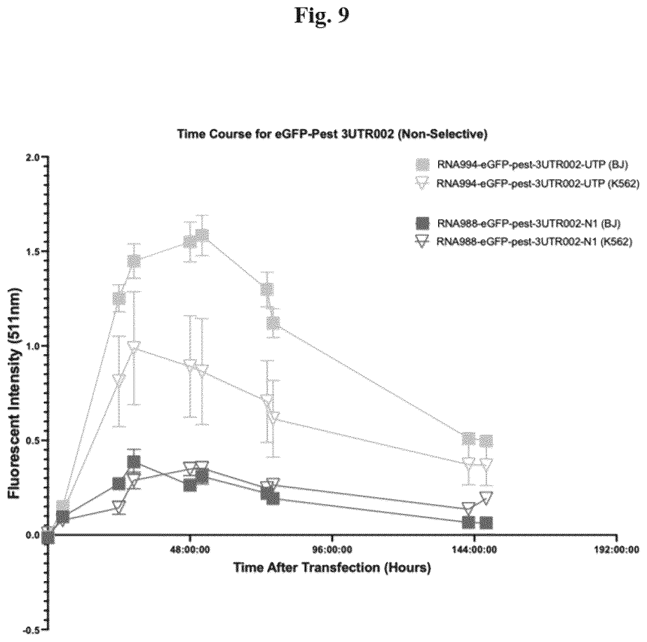

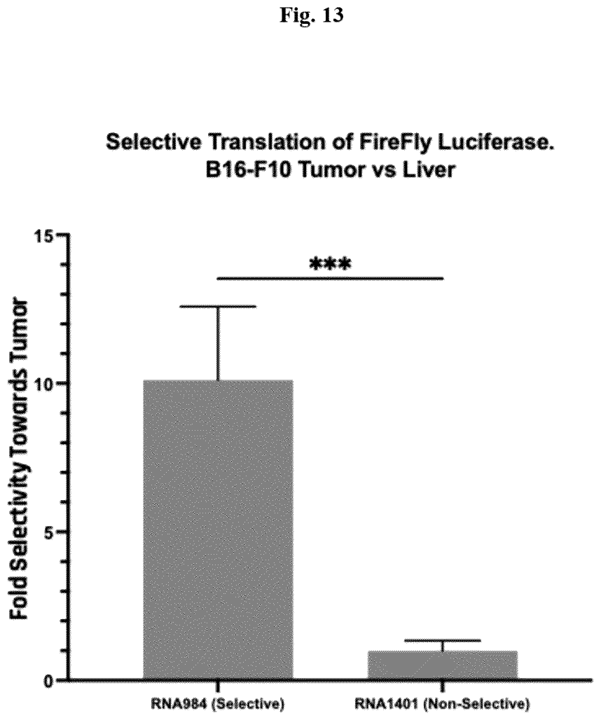

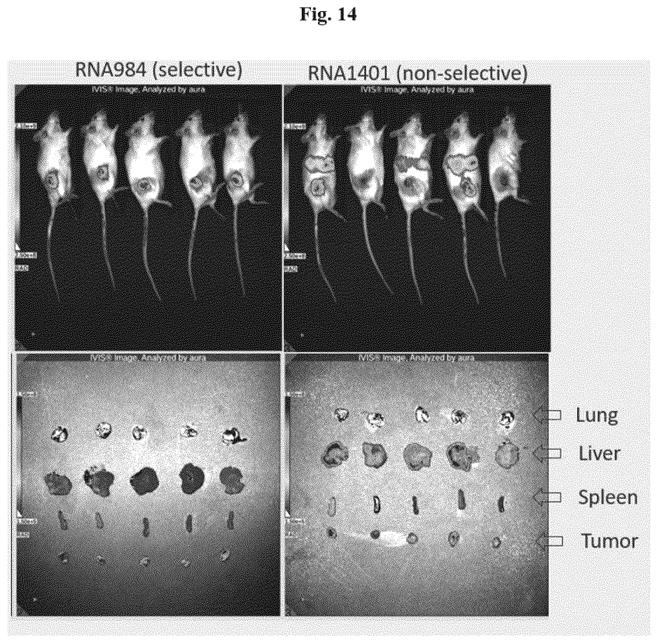

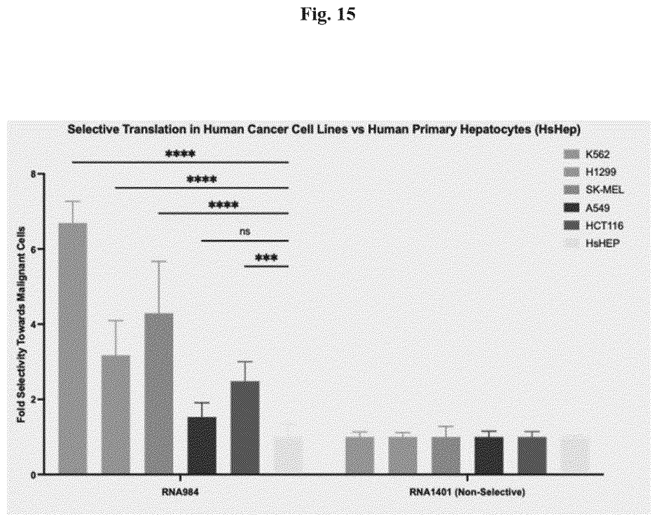

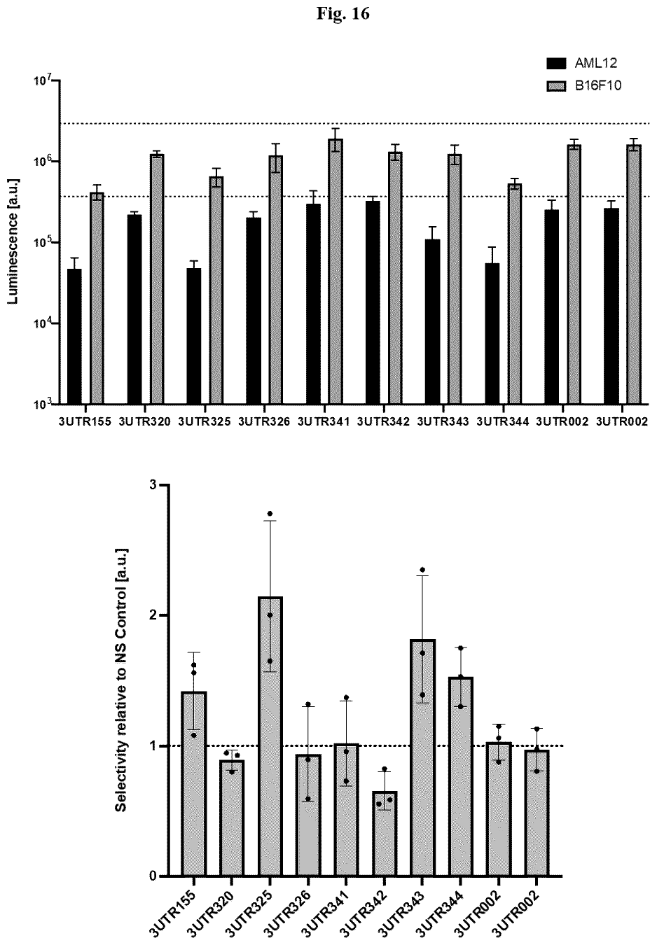

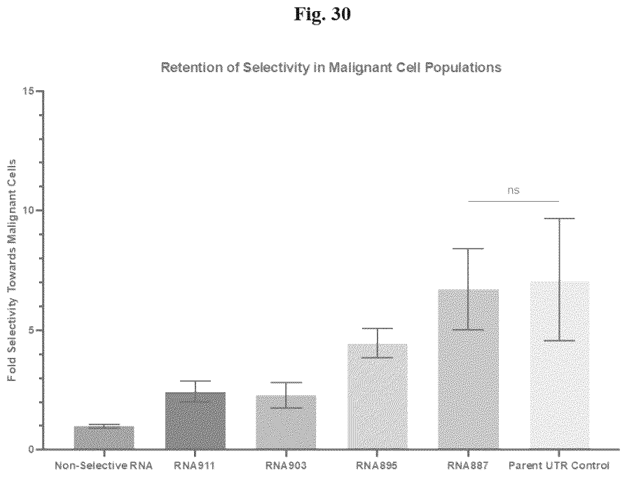

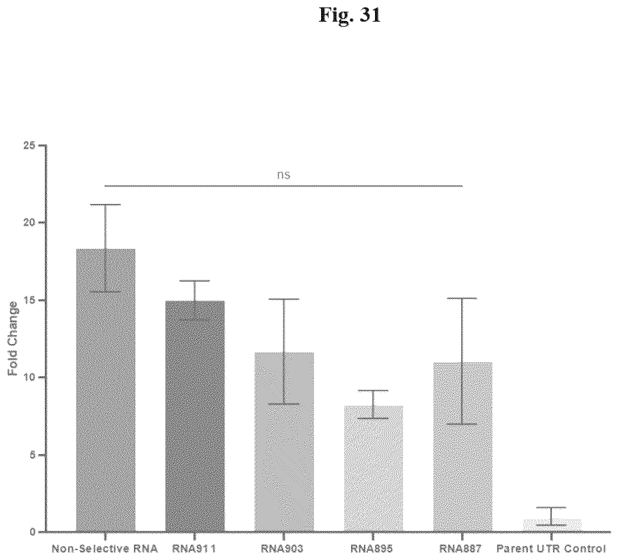

In some embodiments, the method comprises contacting the engineered polynucleotide with a lipid such as a LNP described herein to form a composition described herein, where the composition can be subsequently administered to the subject. As shown in FIG. 7, FIG. 8, and FIG. 15, the composition comprising the engineered polynucleotide comprising the at least one oncoselective sequence (the LNP formulation) does not increase protein expression in liver cell but in a cancer cell. FIG. 13 and FIG. 14 illustrate similar expression specificity (e.g., no expression increase in liver cell but increased expression in cancer cell) in a tumor mouse model. FIGS. 9-12 illustrate that the expression level mediated by the at least one oncoselective sequence can be sustained. In some embodiments, the oncoselective sequence comprises a truncation for increasing expression in cancer cell (e.g., as seen in FIGS. 16-20 and FIG. 32). FIG. 30 and FIG. 31 illustrate that the increased expression mediated by the at least one oncoselective sequence does not decrease the selectivity of the expression. In some embodiments, the method comprising expressing the therapeutic mediated by the at least one oncoselective sequence increases an expression of the engineered therapeutic in a cancer cell compared to a comparable expression of the engineered therapeutic in a non-cancer cell. In some embodiments, the at least one oncoselective sequence increases the expression of the engineered therapeutic in a cancer cell compared to the comparable expression of the engineered therapeutic in a non-cancer cell by at least 10%, at least 20%, at least 30%, at least 40%, at least 50%, at least 100%, at least 200%, at least 500%, or more. In some embodiments, the at least one oncoselective sequence increases a killing efficiency of a cancer cell compared to a comparable killing efficiency of a non-cancer cell.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 1⁰⁰% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Embodiments

Embodiment 1. An engineered polynucleotide comprising: at least one oncoselective sequence; and a coding sequence encoding an engineered therapeutic, wherein the at least one oncoselective sequence comprises a nucleic acid sequence that is: at least 75% identical to any one of SEQ ID NOs: 1-75; or any one of SEQ ID NOs: 76-90.

Embodiment 2. The engineered polynucleotide of Embodiment 1, wherein the at least one oncoselective sequence comprises the nucleic acid sequence that is at least 75% identical to any one of SEQ ID NOs: 1-8.

Embodiment 3. The engineered polynucleotide of Embodiment 2, wherein the at least one oncoselective sequence comprises the nucleic acid sequence that is at least 80%, at least 85%, at least 90%, at least 95%, or at least 99% identical to any one of SEQ ID NOs: 1-8.

Embodiment 4. The engineered polynucleotide of Embodiment 3, wherein the at least one oncoselective sequence comprises the nucleic acid sequence that is any one of SEQ ID NOs: 1-8.

Embodiment 5. The engineered polynucleotide of Embodiment 1, wherein the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-75.

Embodiment 6. The engineered polynucleotide of Embodiment 5, wherein the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-8.

Embodiment 7. The engineered polynucleotide of Embodiment 1, wherein the at least one oncoselective sequence comprises the nucleic acid sequence that is at least 5 contiguous nucleotides identical to any one of SEQ ID NOs: 76-90.

Embodiment 8. The engineered polynucleotide of Embodiment 1, wherein the at least one oncoselective sequence comprises an untranslated region (UTR).

Embodiment 9. The engineered polynucleotide of any one of Embodiments 1-8, wherein the at least one oncoselective sequence a nucleic acid motif.

Embodiment 10. The engineered polynucleotide of Embodiment 1, further comprising two oncoselective sequences.

Embodiment 11. The engineered polynucleotide of Embodiment 10, wherein the two oncoselective sequences flank the coding sequence.

Embodiment 12. The engineered polynucleotide of any one of Embodiments 1-11, wherein the at least one oncoselective sequence comprises at least one miRNA binding site.

Embodiment 13. The engineered polynucleotide of any one of Embodiments 1-12, wherein the at least one oncoselective sequence comprises at least one protein binding site.

Embodiment 14. The engineered polynucleotide of Embodiment 13, wherein the at least one protein binding site is a RNA binding protein (RBP) site.

Embodiment 15. The engineered polynucleotide of any one of Embodiments 1-14, further comprising at least one nucleic acid modification.

Embodiment 16. The engineered polynucleotide of Embodiment 15, wherein the at least one nucleic acid modification comprises a methylation.

Embodiment 17. The engineered polynucleotide of Embodiment 15, wherein the at least one nucleic acid modification comprises a pseudouridine.

Embodiment 18. The engineered polynucleotide of Embodiment 15, wherein the at least one nucleic acid modification comprises an N1-Methylpseudouridine.

Embodiment 19. The engineered polynucleotide of any one of Embodiments 1-18, wherein the engineered therapeutic comprises a cytokine.

Embodiment 20. The engineered polynucleotide of Embodiment 19, wherein the cytokine comprises an engineered cytokine comprising a secreted cytokine, a membrane tethered cytokine, a masked cytokine, a cytokine fusion, or a combination thereof.

Embodiment 21. The engineered polynucleotide of Embodiment 20, wherein the cytokine comprises an interleukin or an interferon.

Embodiment 22. The engineered polynucleotide of Embodiment 21, wherein the cytokine comprises the interleukin.

Embodiment 23. The engineered polynucleotide of Embodiment 22, wherein the interleukin comprises an IL-2.

Embodiment 24. The engineered polynucleotide of Embodiment 22, wherein the interleukin comprises an IL-12.

Embodiment 25. The engineered polynucleotide of Embodiment 22, wherein the interleukin comprises an IL-15.

Embodiment 26. The engineered polynucleotide of Embodiment 21, wherein the cytokine comprises the interferon.

Embodiment 27. The engineered polynucleotide of Embodiment 26, wherein the interferon comprises an IFN-α.

Embodiment 28. The engineered polynucleotide of any one of Embodiments 1-27, wherein the at least one oncoselective sequence comprises a secondary structure.

Embodiment 29. The engineered polynucleotide of Embodiment 28, wherein the secondary structure comprises a stem loop; a bulge loop, a pseudoknot, or a combination thereof.

Embodiment 30. The engineered polynucleotide of Embodiment 29, wherein the stem loop comprises a double stem loop.

Embodiment 31. The engineered polynucleotide of any one of Embodiments 1-30, wherein the at least one oncoselective sequence comprises a truncation.

Embodiment 32. The engineered polynucleotide of any one of Embodiments 1-31, wherein the engineered polynucleotide comprises RNA.

Embodiment 33. The engineered polynucleotide of any one of Embodiments 1-32, wherein the engineered polynucleotide decreases immunogenicity.

Embodiment 34. The engineered polynucleotide of any one of Embodiments 1-33, wherein the at least one oncoselective sequence increases expression of the engineered therapeutic in a cancer cell compared to expression of the engineered therapeutic in a normal cell.

Embodiment 35. A composition comprising the engineered polynucleotide of any one of Embodiments 1-34.

Embodiment 36. A composition comprising two or more of the engineered polynucleotides of any one of Embodiments 1-34.

Embodiment 37. The composition of Embodiment 36, wherein the two or more of the engineered polynucleotides encode two or more cytokines comprising IL-2, IL-12, IL-15, IFN-α, or a combination thereof.

Embodiment 38. The composition of Embodiment 36, wherein the two or more of the engineered polynucleotides encode two or more cytokines comprising IL-2, IL-12, IL-15, and IFN-α.

Embodiment 39. The composition of any one of Embodiments 35-38, further comprising at least one additional active ingredient.

Embodiment 40. The composition of Embodiment 39, wherein the at least one additional active ingredient comprises an immune checkpoint inhibitor.

Embodiment 41. The composition of Embodiment 40, wherein the immune checkpoint inhibitor comprises one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, CD137, or a combination thereof.

Embodiment 42. The composition of Embodiment 40, wherein the immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab.

Embodiment 43. The composition of any one of Embodiments 35-42, wherein the composition comprises contacting the engineered polynucleotide with a lipid.

Embodiment 44. The composition of Embodiment 43, wherein the lipid comprises a lipid nanoparticle (LNP).

Embodiment 45. A vector encoding the engineered polynucleotide of any one of Embodiments 1-34.

Embodiment 46. A cell comprising the vector of Embodiment 45 or the engineered polynucleotide of any one of Embodiments 1-34.

Embodiment 47. The cell of Embodiment 46 comprises an autologous cell or an allogenic cell.

Embodiment 48. A pharmaceutical composition comprising: the engineered polynucleotide of any one of Embodiments 1-34, the composition of any one of Embodiments 35-44, the vector of Embodiment 45, or the cell of Embodiment 46 or 47; and at least one carrier, excipient, or diluent.

Embodiment 49. The pharmaceutical composition of Embodiment 48, wherein the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, or a combination thereof.

Embodiment 50. The pharmaceutical composition of Embodiment 48, wherein the pharmaceutical composition comprises at least one additional active ingredient.

Embodiment 51. A method of administering an engineered polynucleotide a subject, comprising administering the engineered polynucleotide of any one of Embodiments 1-34, the composition of any one of Embodiments 35-44, the vector of Embodiment 45, the cell of Embodiment 46 or 47, or the pharmaceutical composition of any one of Embodiments 47-50 to the subject.

Embodiment 52. A method for treating a disease or condition in a subject comprising administering the engineered polynucleotide of any one of Embodiments 1-34, the composition of any one of Embodiments 35-44, the vector of Embodiment 45, the cell of Embodiment 46 or 47, or the pharmaceutical composition of any one of Embodiments 47-50 to the subject, thereby treating the disease of condition.

Embodiment 53. A method for treating a disease or condition in a subject, comprising administering to the subject an engineered polynucleotide, said engineered polynucleotide comprises at least one oncoselective sequence; and a coding sequence encoding an engineered therapeutic, wherein the at least one oncoselective sequence comprises a nucleic acid sequence that is: at least 75% identical to any one of SEQ ID NOs: 1-75; or any one of SEQ ID NOs: 76-90.

Embodiment 54. The method of Embodiment 53, wherein the at least one oncoselective sequence comprises the nucleic acid sequence that is at least 75% identical to any one of SEQ ID NOs: 1-8.

Embodiment 55. The method of Embodiment 54, wherein the at least one oncoselective sequence comprises the nucleic acid sequence that is at least 80%, at least 85%, at least 90%, at least 95%, or at least 99% identical to any one of SEQ ID NOs: 1-8.

Embodiment 56. The method of Embodiment 55, wherein the at least one oncoselective sequence comprises the nucleic acid sequence that is any one of SEQ ID NOs: 1-8.

Embodiment 57. The method of Embodiment 53, wherein the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-75.

Embodiment 58. The method of Embodiment 57, wherein the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-8.

Embodiment 59. The method of Embodiment 53, wherein the at least one oncoselective sequence comprises the nucleic acid sequence that is at least 5 contiguous nucleotides identical to any one of SEQ ID NOs: 76-90.

Embodiment 60. The method of Embodiment 53, wherein the engineered therapeutic comprises a cytokine.

Embodiment 61. The engineered polynucleotide of Embodiment 60, wherein the cytokine comprises an engineered cytokine comprising a secreted cytokine, a membrane tethered cytokine, a masked cytokine, a cytokine fusion, or a combination thereof.

Embodiment 62. The method of Embodiment 60, wherein the cytokine comprises an interleukin or an interferon.

Embodiment 63. The method of Embodiment 62, wherein the cytokine comprises the interleukin.

Embodiment 64. The method of Embodiment 63, wherein the interleukin comprises an IL-2.

Embodiment 65. The method of Embodiment 63, wherein the interleukin comprises an IL-12.

Embodiment 66. The method of Embodiment 63, wherein the interleukin comprises an IL-15.

Embodiment 67. The method of Embodiment 62, wherein the interferon comprises an IFN-α.

Embodiment 68. The method of Embodiment 53, wherein the disease or condition comprises solid tumor.

Embodiment 69. The method of Embodiment 53, wherein the disease or condition comprises cancer.

Embodiment 70. The method of Embodiment 69, wherein the cancer comprises melanoma, breast cancer, basal cell carcinoma (BCC), squamous cell carcinoma (SCC), cutaneous SCC (cSCC or CSCC), Head & Neck Cancer, Thyroid Cancer, Colorectal Cancer, Prostate Cancer, Liver cancer, Pancreatic Cancer, Renal Cell Carcinoma, Brain Cancer, Soft Tissue Sarcoma, Lung Cancer, or a combination thereof.

Embodiment 71. The method of Embodiment 69, wherein the cancer does not respond to immune checkpoint inhibitor treatment.

Embodiment 72. The method of Embodiment 53, wherein the at least one oncoselective sequence increases expression of the engineered therapeutic in a cancer cell compared to expression of the engineered therapeutic in a normal cell.

Embodiment 73. An engineered polynucleotide comprising: at least one oncoselective sequence; and a coding sequence encoding an engineered therapeutic.

Embodiment 74. The engineered polynucleotide of Embodiment 2, wherein the at least one oncoselective sequence comprises a nucleic acid sequence that is at least 75%, at least 80%, at least 85%, at least 90%, at least 95%, or at least 99% identical to any one of SEQ ID NOs: 101-146.

Embodiment 75. The engineered polynucleotide of Embodiment 74, wherein the at least one oncoselective sequence comprises a nucleic acid sequence that is at least 50 contiguous nucleotides, at least 100 contiguous nucleotides, or at least 150 contiguous nucleotides identical to any one of SEQ ID NOs: 101-146.

Embodiment 76. The engineered polynucleotide of Embodiment 73, further comprising two oncoselective sequences.

Embodiment 77. The engineered polynucleotide of Embodiment 76, wherein the two oncoselective sequences flank the coding sequence.

Embodiment 78. The engineered polynucleotide of any one of Embodiments 73-77, wherein the at least one oncoselective sequence comprises at least one miRNA binding site.

Embodiment 79. The engineered polynucleotide of any one of Embodiments 73-77, wherein the at least one oncoselective sequence comprises at least one protein binding site.

Embodiment 80. The engineered polynucleotide of Embodiment 79, wherein the at least one protein binding site is a RNA binding protein (RBP) site.

Embodiment 81. The engineered polynucleotide of any one of Embodiments 73-80, further comprising at least one nucleic acid modification.

Embodiment 82. The engineered polynucleotide of Embodiment 81, wherein the at least one nucleic acid modification comprises a methylation.

Embodiment 83. The engineered polynucleotide of Embodiment 81, wherein the at least one nucleic acid modification comprises a pseudouridine.

Embodiment 84. The engineered polynucleotide of Embodiment 81, wherein the at least one nucleic acid modification comprises an N1-Methylpseudouridine.

Embodiment 85. The engineered polynucleotide of any one of Embodiments 73-84, wherein the at least one oncoselective sequence comprises a secondary structure.

Embodiment 86. The engineered polynucleotide of Embodiment 85, wherein the secondary structure comprises a stem loop; a bulge loop, a pseudoknot, or a combination thereof.

Embodiment 87. The engineered polynucleotide of Embodiment 86, wherein the stem loop comprises a double stem loop.

Embodiment 88. The engineered polynucleotide of any one of Embodiments 73-87, wherein the at least one oncoselective sequence comprises a truncation.

Embodiment 89. The engineered polynucleotide of any one of Embodiments 73-88, wherein the engineered polynucleotide comprises RNA.

Embodiment 90. The engineered polynucleotide of any one of Embodiments 73-89, wherein the engineered polynucleotide comprises reduced immunogenicity.

Embodiment 91. The engineered polynucleotide of any one of Embodiments 73-90, wherein the at least one oncoselective sequence increases expression of the engineered therapeutic in a cancer cell compared to expression of the engineered therapeutic in a normal cell.

Embodiment 92. A composition comprising the engineered polynucleotide of any one of Embodiments 73-91.

Embodiment 93. The composition of Embodiment 92, wherein the composition comprises contacting the engineered polynucleotide with a lipid.

Embodiment 94. The composition of Embodiment 93, wherein the lipid comprises a lipid nanoparticle (LNP).

Embodiment 95. A vector encoding the engineered polynucleotide of any one of Embodiments 73-94.

Embodiment 96. A cell comprising the vector of Embodiment 95 or the engineered polynucleotide of any one of Embodiments 73-94.

Embodiment 97. The cell of Embodiment 96 comprises an autologous cell or an allogenic cell.

Embodiment 98. A pharmaceutical composition comprising: the engineered polynucleotide of any one of Embodiments 73-91, the composition of any one of Embodiments 92-94, the vector of Embodiment 95, or the cell of Embodiment 96 or 97; and at least one carrier, excipient, or diluent.

Embodiment 99. The pharmaceutical composition of Embodiment 98, wherein the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, or a combination thereof.

Embodiment 100. The pharmaceutical composition of Embodiment 98, further comprising at least one additional active ingredient.

Embodiment 101. A method of administering an engineered polynucleotide a subject, comprising administering the engineered polynucleotide of any one of Embodiments 73-91, the composition of any one of Embodiments 92-94, the vector of Embodiment 95, the cell of Embodiment 96 or 97, or the pharmaceutical composition of any one of Embodiments 98-100 to the subject.

Embodiment 102. A method for treating a disease or condition in a subject, comprising administering the engineered polynucleotide of any one of Embodiments 73-91, the composition of any one of Embodiments 92-94, the vector of Embodiment 95, the cell of Embodiment 96 or 97, or the pharmaceutical composition of any one of Embodiments 98-100 to the subject, thereby treating the disease of condition.

Embodiment 103. A method for treating a disease or condition in a subject, comprising administering to the subject an engineered polynucleotide, said engineered polynucleotide comprises: at least one oncoselective sequence; and a coding sequence encoding an engineered therapeutic.

Embodiment 104. The method of Embodiment 103, wherein the at least one oncoselective sequence comprises an untranslated region (UTR).

Embodiment 105. The method of Embodiment 104, wherein the UTR is a 5′ UTR or a 3′ UTR.

Embodiment 106. The method of any one of Embodiments 103-105, wherein the at least one oncoselective sequence comprises a nucleic acid sequence that is at least 75%, at least 80%, at least 85%, at least 90%, at least 95%, or at least 99% identical to any one SEQ ID NOs: 101-146.

Embodiment 107. The method of any one of Embodiments 103-105, wherein the at least one oncoselective sequence comprises a nucleic acid sequence that is at least 50 contiguous nucleotides, at least 100 contiguous nucleotides, or at least 150 contiguous nucleotides identical to any one of SEQ ID NOs: 101-146.

Embodiment 108. The method of Embodiment 103, wherein the engineered polynucleated comprises two oncoselective sequences.

Embodiment 109. The method of Embodiment 108, wherein the two oncoselective sequences flank the coding sequence.

Embodiment 110. The method of any one of Embodiments 103-109, wherein the at least one oncoselective sequence comprises at least one miRNA binding site.

Embodiment 111. The method of any one of Embodiments 103-109, wherein the at least one oncoselective sequence comprises at least one protein binding site.

Embodiment 112. The method of Embodiment 111, wherein the at least one protein binding site is a RNA binding protein (RBP) site.

Embodiment 113. The method of any one of Embodiments 103-112, wherein the engineered polynucleotide comprises at least one nucleic acid modification.

Embodiment 114. The method of Embodiment 113, wherein the at least one nucleic acid modification comprises a methylation.

Embodiment 115. The method of Embodiment 113, wherein the at least one nucleic acid modification comprises a pseudouridine.

Embodiment 116. The method of Embodiment 113, wherein the at least one nucleic acid modification comprises an N1-Methylpseudouridine.

Embodiment 117. The method of any one of Embodiments 103-116, wherein the at least one oncoselective sequence comprises a secondary structure.

Embodiment 118. The method of Embodiment 117, wherein the secondary structure comprises a stem loop; a bulge loop, a pseudoknot, or a combination thereof.

Embodiment 119. The method of Embodiment 118, wherein the stem loop comprises a double stem loop.

Embodiment 120. The method of any one of Embodiments 103-119, wherein the at least one oncoselective sequence comprises a truncation.

Embodiment 121. The method of any one of Embodiments 103-120, wherein the engineered polynucleotide comprises RNA.

Embodiment 122. The method of any one of Embodiments 103-121, wherein the engineered polynucleotide comprises reduced immunogenicity.

Embodiment 123. The method of any one of Embodiments 103-122, wherein the at least one oncoselective sequence increases expression of the engineered therapeutic in a cancer cell compared to expression of the engineered therapeutic in a normal cell.

Embodiment 124. The method of any one of Embodiments 103-123, wherein the disease or condition comprises solid tumor.

Embodiment 125. The method of Embodiment 103, wherein the disease or condition comprises cancer.

Embodiment 126. The method of Embodiment 125, wherein the cancer does not respond to immune checkpoint inhibitor treatment.

Embodiment 131. An engineered polynucleotide comprising: at least one oncoselective sequence; and a coding sequence encoding an engineered therapeutic.

Embodiment 132. The engineered polynucleotide of Embodiment 131, wherein the engineered therapeutic comprises: an engineered interleukin or fragment thereof; or an engineered subunit of an interleukin.

Embodiment 133. The engineered polynucleotide of Embodiment 132, wherein the engineered interleukin or fragment thereof comprises an engineered IL-12 or fragment thereof or an engineered subunit of LL-12.

Embodiment 134. The engineered polynucleotide of Embodiment 132, wherein the engineered interleukin or fragment thereof comprises an engineered IL-2 or fragment thereof.

Embodiment 135. The engineered polynucleotide of any one of Embodiments 131-134, wherein the engineered therapeutic comprises a masking domain.

Embodiment 136. The engineered polynucleotide of any one of Embodiments 131-135, wherein the engineered therapeutic comprises a half-life extension domain.

Embodiment 137. The engineered polynucleotide of Embodiment 136, wherein the half-life extension domain comprises antibody or fragment thereof a serum binding protein or fragment thereof.

Embodiment 138. The engineered polynucleotide of any one of Embodiments 131-137, wherein the engineered therapeutic comprises a linker.

Embodiment 139. The engineered polynucleotide of Embodiment 138, wherein the linker is a cleavable linker.

Embodiment 140. The engineered polynucleotide of any one of Embodiments 131-139, wherein the engineered therapeutic comprises a signal peptide.

Embodiment 141. The engineered polynucleotide of any one of Embodiments 131-140, wherein the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, 648-663, 351-361, 371-373, 481, 482, 614-621, or 664-667.

Embodiment 142. The engineered polynucleotide of Embodiment 141, wherein the engineered therapeutic comprises an amino acid sequence that is any one of SEQ ID NOs: 301-303, 311-314, 321-328, 331-335, 341-344, 483-487, 648-663, 351-361, 371-373, 481, 482, 614-621, or 664-667.

Embodiment 143. The engineered polynucleotide of any one of Embodiments 131-140, wherein the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 301-303, 311-314, 321-328, 331-335, 341-344, 483-487, 648-663, 351-361, 371-373, 481, 482, 614-621, or 664-667.

Embodiment 144. The engineered polynucleotide of any one of Embodiments 131-140, wherein the engineered therapeutic comprises an amino acid sequence that is at least 75%, at least 80%, at least 85%, at least 90%, at least 95%, or at least 99% identical to any one of SEQ ID NOs: 301-303.

Embodiment 145. The engineered polynucleotide of Embodiment 144, wherein the engineered therapeutic comprises an amino acid sequence that is at any one of SEQ ID NOs: 301-303.

Embodiment 146. The engineered polynucleotide of any one of Embodiments 131-140, wherein the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 301-303.

Embodiment 147. The engineered polynucleotide of any one of Embodiments 131-140, wherein the engineered therapeutic comprises an amino acid sequence that is at least 75%, at least 80%, at least 85%, at least 90%, at least 95%, or at least 99% identical to any one of SEQ ID NOs: 311-314 or 321-328.

Embodiment 148. The engineered polynucleotide of Embodiment 147, wherein the engineered therapeutic comprises an amino acid sequence that is at any one of SEQ ID NOs: 311-314 or 321-328.

Embodiment 149. The engineered polynucleotide of any one of Embodiments 131-140, wherein the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 311-314 or 321-328.

Embodiment 150. The engineered polynucleotide of any one of Embodiments 131-140, wherein the engineered therapeutic comprises an amino acid sequence that is at least 75%, at least 80%, at least 85%, at least 90%, at least 95%, or at least 99% identical to any one of SEQ ID NOs: 351-361, 371-373, 481, 482, 614-621, or 664-667.

Embodiment 151. The engineered polynucleotide of Embodiment 150, wherein the engineered therapeutic comprises an amino acid sequence that is at any one of 351-361, 371-373, 481, 482, 614-621, or 664-667.

Embodiment 152. The engineered polynucleotide of any one of Embodiments 131-140, wherein the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 351-361, 371-373, 481, 482, 614-621, or 664-667.

Embodiment 153. The engineered polynucleotide of any one of Embodiments 131-152, wherein the at least one oncoselective sequence comprises a nucleic acid motif.

Embodiment 154. The engineered polynucleotide of Embodiment 153, wherein the nucleic acid motif comprises an oncoselective readthrough motif.

Embodiment 155. The engineered polynucleotide of any one of Embodiments 131-154, wherein the at least one oncoselective sequence comprises an untranslated region (UTR).

Embodiment 156. The engineered polynucleotide of Embodiment 155, wherein the UTR is a 5′ UTR or a 3′ UTR.

Embodiment 157. The engineered polynucleotide of any one of Embodiments 131-156, wherein the at least one oncoselective sequence comprises a nucleic acid sequence that is at least 75%, at least 80%, at least 85%, at least 90%, at least 95%, or at least 99% identical to any one of SEQ ID NOs: 1-75, 91-97, or 101-146; or any one of SEQ ID NOs: 76-90.

Embodiment 158. The engineered polynucleotide of any one of Embodiments 131-156, wherein the at least one oncoselective sequence comprises a nucleic acid sequence that is at least 50 contiguous nucleotides, at least 100 contiguous nucleotides, or at least 150 contiguous nucleotides identical to any one of SEQ ID NOs: 1-75, 91-97, or 101-146; or at least 5 contiguous nucleotides identical to any one of SEQ ID NOs: 76-90.

Embodiment 159. The engineered polynucleotide of Embodiment 131, further comprising two oncoselective sequences.

Embodiment 160. The engineered polynucleotide of Embodiment 159, wherein the two oncoselective sequences flank the coding sequence.

Embodiment 161. The engineered polynucleotide of any one of Embodiments 131-160, wherein the at least one oncoselective sequence comprises at least one miRNA binding site.

Embodiment 162. The engineered polynucleotide of any one of Embodiments 131-161, wherein the at least one oncoselective sequence comprises at least one protein binding site.

Embodiment 163. The engineered polynucleotide of Embodiment 162, wherein the at least one protein binding site is a RNA binding protein (RBP) site.

Embodiment 164. The engineered polynucleotide of any one of Embodiments 131-163, further comprising at least one nucleic acid modification.

Embodiment 165. The engineered polynucleotide of Embodiment 164, wherein the at least one nucleic acid modification comprises a methylation.

Embodiment 166. The engineered polynucleotide of Embodiment 164, wherein the at least one nucleic acid modification comprises a pseudouridine.

Embodiment 167. The engineered polynucleotide of Embodiment 164, wherein the at least one nucleic acid modification comprises an N1-Methylpseudouridine.

Embodiment 168. The engineered polynucleotide of Embodiment 131, wherein the engineered therapeutic comprises an engineered cytokine comprising modified cytokine comprising a secreted cytokine, a membrane tethered cytokine, a masked cytokine, a cytokine fusion, or a combination thereof.

Embodiment 169. The engineered polynucleotide of Embodiment 168, wherein the cytokine fusion comprises a cytokine coupled to a half-life extension domain.

Embodiment 170. The engineered polynucleotide of Embodiment 169, wherein the half-life extension domain comprises an antibody or fragment thereof or a serum binding protein or fragment thereof.

Embodiment 171. The engineered polynucleotide of Embodiment 168, wherein the engineered cytokine comprises an interleukin or an interferon.

Embodiment 172. The engineered polynucleotide of Embodiment 171, wherein the engineered cytokine comprises the interleukin.

Embodiment 173. The engineered polynucleotide of Embodiment 172, wherein the interleukin comprises an IL-2.

Embodiment 174. The engineered polynucleotide of Embodiment 172, wherein the interleukin comprises an IL-12.

Embodiment 175. The engineered polynucleotide of Embodiment 172, wherein the interleukin comprises an IL-15.

Embodiment 176. The engineered polynucleotide of Embodiment 171, wherein the interferon comprises an IFN-α.

Embodiment 177. The engineered polynucleotide of any one of Embodiments 131-176, wherein the at least one oncoselective sequence comprises a secondary structure.

Embodiment 178. The engineered polynucleotide of Embodiment 177, wherein the secondary structure comprises a stem loop; a bulge loop, a pseudoknot, or a combination thereof.

Embodiment 179. The engineered polynucleotide of Embodiment 177, wherein the stem loop comprises a double stem loop.

Embodiment 180. The engineered polynucleotide of any one of Embodiments 131-179, wherein the at least one oncoselective sequence comprises a truncation.

Embodiment 181. The engineered polynucleotide of any one of Embodiments 131-180, wherein the engineered polynucleotide comprises RNA.

Embodiment 182. The engineered polynucleotide of any one of Embodiments 131-181, wherein the engineered polynucleotide comprises reduced immunogenicity.

Embodiment 183. The engineered polynucleotide of any one of Embodiments 131-182, wherein the at least one oncoselective sequence increases expression of the engineered therapeutic in a cancer cell compared to expression of the engineered therapeutic in a normal cell.

Embodiment 184. A composition comprising the engineered polynucleotide of any one of Embodiments 131-183.

Embodiment 185. The composition of Embodiment 184, further comprising at least one additional polynucleotide encoding a cytokine.

Embodiment 186. The composition of Embodiment 55, wherein the cytokine comprises IL-2, IL-12, IL-15, IFN-α, or a combination thereof.

Embodiment 187. A composition comprising two or more of the engineered polynucleotide of any one of Embodiments 131-186.

Embodiment 188. The composition of Embodiment 187, wherein the two or more of the engineered polynucleotide encode two or more of the engineered therapeutic.

Embodiment 189. The composition of Embodiment 188, wherein the two or more of the engineered therapeutic comprise: an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, or 648-663; and an amino acid sequence that is at least 75%, at least 80%, at least 85%, at least 90%, at least 95%, or at least 99% identical to any one of SEQ ID NOs: 351-361, 371-373, 481, 482, 614-621, or 664-667.

Embodiment 190. The composition of Embodiment 188, wherein the two or more of the engineered therapeutic comprise: an amino acid sequence that is at least 75%, at least 80%, at least 85%, at least 90%, at least 95%, or at least 99% identical to any one of SEQ ID NOs: 311, 313, 321, 323, 325, or 327; an amino acid sequence that is at least 75%, at least 80%, at least 85%, at least 90%, at least 95%, or at least 99% identical to any one of SEQ ID NOs: 312, 314, 322, 324, 326, or 328; and an amino acid sequence that is at least 75%, at least 80%, at least 85%, at least 90%, at least 95%, or at least 99% identical to any one of SEQ ID NOs: 351-361, 371-373, 481, 482, 614-621, or 664-667.

Embodiment 191. The composition of any one of Embodiments 187-190, further comprising at least one additional polynucleotide encoding a cytokine.

Embodiment 192. The composition of Embodiment 191, wherein the cytokine comprises IL-2, IL-12, IL-15, IFN-α, or a combination thereof.

Embodiment 193. The composition of any one of Embodiments 184-192, further comprising at least one additional active ingredient.

Embodiment 194. The composition of Embodiment 193, wherein the at least one additional active ingredient comprises an immune checkpoint inhibitor.

Embodiment 195. The composition of Embodiment 194, wherein the immune checkpoint inhibitor comprises one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, CD137, or a combination thereof.

Embodiment 196. The composition of Embodiment 194, wherein the immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab.

Embodiment 197. The composition of any one of Embodiments 184-196, wherein the composition comprises contacting the engineered polynucleotide with a lipid.

Embodiment 198. The composition of Embodiment 197, wherein the lipid comprises a lipid nanoparticle (LNP).

Embodiment 199. A vector encoding the engineered polynucleotide of any one of Embodiments 131-186.

Embodiment 200. A cell comprising the vector of Embodiment 199 or the engineered polynucleotide of any one of Embodiments 131-183.

Embodiment 201. The cell of Embodiment 200 comprises an autologous cell or an allogenic cell.

Embodiment 202. A pharmaceutical composition comprising: the engineered polynucleotide of any one of Embodiments 131-183, the composition of any one of Embodiments 183-198, the vector of Embodiment 199, or the cell of Embodiment 200 or 201; and at least one carrier, excipient, or diluent.

Embodiment 203. The pharmaceutical composition of Embodiment 202, wherein the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, or a combination thereof.

Embodiment 204. The pharmaceutical composition of Embodiment 201, further comprising at least one additional active ingredient.

Embodiment 205. A method of administering an engineered polynucleotide a subject, comprising administering the engineered polynucleotide of any one of Embodiments 131-183, the composition of any one of Embodiments 184-198, the vector of Embodiment 199, the cell of Embodiment 200 or 201, or the pharmaceutical composition of any one of Embodiments 202-204 to the subject.

Embodiment 206. A method for treating a disease or condition in a subject, comprising administering the engineered polynucleotide of any one of Embodiments 131-183, the composition of any one of Embodiments 184-198, the vector of Embodiment 199, the cell of Embodiment 200 or 201, or the pharmaceutical composition of any one of Embodiments 202-204 to the subject, thereby treating the disease of condition.

Embodiment 207. A method for treating a disease or condition in a subject, comprising administering to the subject an engineered polynucleotide, said engineered polynucleotide comprises: at least one oncoselective sequence; and a coding sequence encoding an engineered therapeutic.

Embodiment 208. The method of Embodiment 207, wherein the engineered therapeutic comprises: an engineered interleukin or fragment thereof; or an engineered subunit of an interleukin.

Embodiment 209. The method of Embodiment 208, wherein the engineered interleukin or fragment thereof comprises an engineered IL-12 or fragment thereof or an engineered subunit of IL-12.

Embodiment 210. The method of Embodiment 208, wherein the engineered interleukin or fragment thereof comprises an engineered IL-2 or fragment thereof.

Embodiment 211. The method of any one of Embodiments 207-210, wherein the engineered therapeutic comprises a masking domain.

Embodiment 212. The method of any one of Embodiments 207-211, wherein the engineered therapeutic comprises a half-life extension domain.

Embodiment 213. The method of Embodiment 212, wherein the half-life extension domain comprises an antibody or fragment thereof a serum binding protein or fragment thereof.

Embodiment 214. The method of any one of Embodiments 207-213, wherein the engineered therapeutic comprises a linker.

Embodiment 215. The method of Embodiment 214, wherein the linker is a cleavable linker.

Embodiment 216. The method of any one of Embodiments 207-215, wherein the engineered therapeutic comprises a signal peptide.

Embodiment 217. The method of any one of Embodiments 207-216, wherein the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 483-487, or 648-663.

Embodiment 218. The method of Embodiment 217, wherein the engineered therapeutic comprises an amino acid sequence that is any one of SEQ ID NOs: 301-303, 311-314, 321-328, 331-335, 341-344, 483-487, or 648-663.

Embodiment 219. The method of any one of Embodiments 207-216, wherein the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 301-303, 311-314, 321-328, 331-335, 341-344, 483-487, or 648-663.

Embodiment 220. The method of any one of Embodiments 207-216, wherein the engineered therapeutic comprises an amino acid sequence that is at least 75%, at least 80%, at least 85%, at least 90%, at least 95%, or at least 99% identical to any one of SEQ ID NOs: 301-303.

Embodiment 221. The method of Embodiment 90, wherein the engineered therapeutic comprises an amino acid sequence that is at any one of SEQ ID NOs: 301-303.

Embodiment 222. The method of any one of Embodiments 207-216, wherein the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 301-303.

Embodiment 223. The method of any one of Embodiments 207-216, wherein the engineered therapeutic comprises an amino acid sequence that is at least 75%, at least 80%, at least 85%, at least 90%, at least 95%, or at least 99% identical to any one of SEQ ID NOs: 311-314 or 321-328.

Embodiment 224. The method of Embodiment 223, wherein the engineered therapeutic comprises an amino acid sequence that is at any one of SEQ ID NOs: 311-314 or 321-328.

Embodiment 225. The method of any one of Embodiments 207-216, wherein the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 311-314 or 321-238.

Embodiment 226. The method of any one of Embodiments 207-216, wherein the engineered therapeutic comprises an amino acid sequence that is at least 75%, at least 80%, at least 85%, at least 90%, at least 95%, or at least 99% identical to any one of SEQ ID NOs: 351-361, 371-373, 481, 482, 614-621, or 664-667.

Embodiment 227. The method of Embodiment 226, wherein the engineered therapeutic comprises an amino acid sequence that is at any one of 351-361, 371-373, 481, 482, 614-621, or 664-667.

Embodiment 228. The method of any one of Embodiments 207-216, wherein the engineered therapeutic comprises an amino acid sequence that is at least 50 contiguous amino acids, at least 100 contiguous amino acids, at least 150 contiguous amino acids, at least 200 contiguous amino acids, at least 250 contiguous amino acids, at least 300 contiguous amino acids, at least 350 contiguous amino acids, at least 400 contiguous amino acids, at least 450 contiguous amino acids, at least 500 contiguous amino acids, at least 550 contiguous amino acids, or at least 600 contiguous amino acids identical to any one of SEQ ID NOs: 351-361, 371-373, 481, 482, 614-621, or 664-667.

Embodiment 229. The method of any one of Embodiments 207-228, wherein the at least one oncoselective sequence comprises a nucleic acid motif.

Embodiment 230. The method of Embodiment 229, wherein the nucleic acid motif comprises an oncoselective readthrough motif.

Embodiment 231. The method of any one of Embodiments 207-230, wherein the at least one oncoselective sequence comprises an untranslated region (UTR).

Embodiment 232. The method of Embodiment 231, wherein the UTR is a 5′ UTR or a 3′ UTR.

Embodiment 233. The method of any one of Embodiments 207-232, wherein the at least one oncoselective sequence comprises a nucleic acid sequence that is at least 75%, at least 80%, at least 85%, at least 90%, at least 95%, or at least 99% identical to any one SEQ ID NOs: 1-75, 91-97, or 101-146; or any one of SEQ ID NOs: 76-90.

Embodiment 234. The method of any one of Embodiments 207-233, wherein the at least one oncoselective sequence comprises a nucleic acid sequence that is at least 50 contiguous nucleotides, at least 100 contiguous nucleotides, or at least 150 contiguous nucleotides identical to any one of SEQ ID NOs: 1-75, 91-97, or 101-146; or at least 5 contiguous nucleotides identical to any one of SEQ ID NOs: 76-90.

Embodiment 235. The method of Embodiment 207, wherein the engineered polynucleated comprises two oncoselective sequences.

Embodiment 236. The method of Embodiment 235, wherein the two oncoselective sequences flank the coding sequence.

Embodiment 237. The method of any one of Embodiments 207-236, wherein the at least one oncoselective sequence comprises at least one miRNA binding site.

Embodiment 238. The method of any one of Embodiments 207-237, wherein the at least one oncoselective sequence comprises at least one protein binding site.

Embodiment 239. The method of Embodiment 238, wherein the at least one protein binding site is a RNA binding protein (RBP) site.

Embodiment 240. The method of any one of Embodiments 207-239, wherein the engineered polynucleotide comprises at least one nucleic acid modification.

Embodiment 241. The method of Embodiment 240, wherein the at least one nucleic acid modification comprises a methylation.

Embodiment 242. The method of Embodiment 240, wherein the at least one nucleic acid modification comprises a pseudouridine.

Embodiment 243. The method of Embodiment 240, wherein the at least one nucleic acid modification comprises an NI-Methylpseudouridine.

Embodiment 244. The method of Embodiment 207, wherein the engineered therapeutic comprises an engineered cytokine comprising modified cytokine comprising a secreted cytokine, a membrane tethered cytokine, a masked cytokine, a cytokine fusion, or a combination thereof.

Embodiment 245. The method of Embodiment 244, wherein the cytokine fusion comprises a cytokine coupled to a half-life extension domain.

Embodiment 246. The method of Embodiment 245, wherein the half-life extension domain comprises an antibody or fragment thereof a serum binding protein or fragment thereof.

Embodiment 247. The method of Embodiment 246, wherein the engineered cytokine comprises an interleukin or an interferon.

Embodiment 248. The method of Embodiment 247, wherein the engineered cytokine comprises the interleukin.

Embodiment 249. The method of Embodiment 248, wherein the interleukin comprises an IL-2.

Embodiment 250. The method of Embodiment 248, wherein the interleukin comprises an IL-12.

Embodiment 251. The method of Embodiment 248, wherein the interleukin comprises an IL-15.

Embodiment 252. The method of Embodiment 247, wherein the interferon comprises an IFN-α.

Embodiment 253. The method of any one of Embodiments 207-252, wherein the at least one oncoselective sequence comprises a secondary structure.

Embodiment 254. The method of Embodiment 253, wherein the secondary structure comprises a stem loop; a bulge loop, a pseudoknot, or a combination thereof.

Embodiment 255. The method of Embodiment 254, wherein the stem loop comprises a double stem loop.

Embodiment 256. The method of any one of Embodiments 207-255, wherein the at least one oncoselective sequence comprises a truncation.

Embodiment 257. The method of any one of Embodiments 207-256, wherein the engineered polynucleotide comprises RNA.

Embodiment 258. The method of any one of Embodiments 207-257, wherein the engineered polynucleotide comprises reduced immunogenicity.

Embodiment 259. The method of any one of Embodiments 207-258, wherein the at least one oncoselective sequence increases expression of the engineered therapeutic in a cancer cell compared to expression of the engineered therapeutic in a normal cell.

Embodiment 260. The method of any one of Embodiments 207-259, wherein the disease or condition comprises solid tumor.

Embodiment 261. The method of Embodiment 260, wherein the disease or condition comprises cancer.

Embodiment 262. The method of Embodiment 261, wherein the cancer comprises melanoma, breast cancer, basal cell carcinoma (BCC), squamous cell carcinoma (SCC), cutaneous SCC (cSCC or CSCC), Head & Neck Cancer, Thyroid Cancer, Colorectal Cancer, Prostate Cancer, Liver cancer, Pancreatic Cancer, Renal Cell Carcinoma, Brain Cancer, Soft Tissue Sarcoma, Lung Cancer, or a combination thereof.

Embodiment 263. The method of Embodiment 261, wherein the cancer does not respond to immune checkpoint inhibitor treatment.

### EXAMPLES

The following illustrative examples are representative of embodiments of the stimulation, systems, and methods described herein and are not meant to be limiting in any way.

### Example 1. Computational Analysis Methods and Pipelines

### Consensus Peptide Database Search and Testing

Search of peptide databases for relevant sequence proceeded using ribosome profiling (Ribo-Seq) is a method which detects the frequency of translating ribosomes across mRNA transcripts. Non-canonical protein sequences (such as from the 3′ and 5′ UTR regions) are not usually included in proteomic analysis due to increased search space decreasing search accuracy. By extracting mRNA UTR regions which ribosome profiling (Ribo-Seq) calls as translated and adding the corresponding peptide sequences to the search space alongside the standard proteome, these sequences can be efficiently detected in proteomic analysis.

A strategy of “consensus search,” in which multiple peptide database search algorithms are applied in parallel, was used to screen databases including MaxQuant, Comet, MS-GF, and Myrimatch. To handle the computational requirements of a consensus search workflow applied to large-scale cancer proteomic datasets, a pipeline platform was developed using multiple peptide search engines. Candidate sequences were elected based on sequences predicted to exhibit selectivity for translation in oncogenic cell lines. FIG. 1 shows overlap in peptide identifications between multiple peptide search engines used in the proteomic pipeline. FIG. 2 shows relative 3′ UTR and 5′ UTR Peptide-Spectrum Matches (PSMs) counts in multi-tissue search results (overall detections including CDS were >1.5×10⁶ PSMs).

### Cellular Assay of K562 and BJ Cell Lines

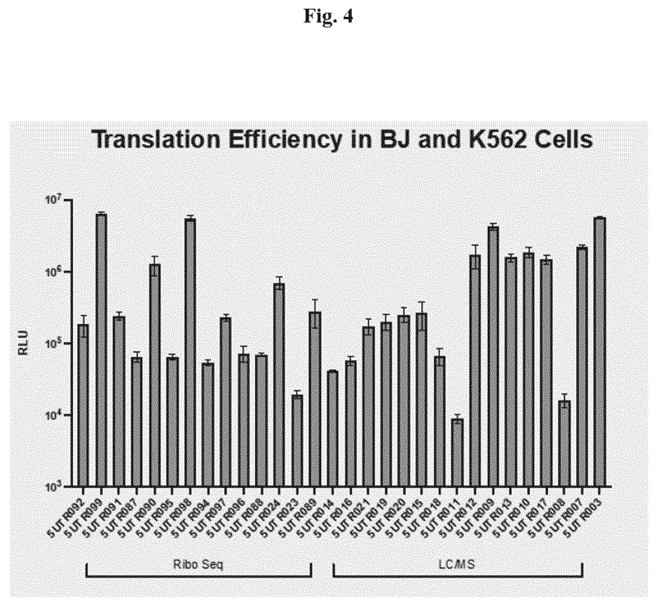

Transfection of 3 independent replicates with 100 ng candidate mRNA was performed using MessengerMax. Selectivity was calculated by taking the ratio of the K562 replicate over the BJ average, then normalizing to the non-selective control (5′ UTR003). Results showed specific elements had selectivity for K562 cells over healthy BJ cells. FIG. 3 and FIG. 4 show the results of sequences screened. The sequences SEQ ID NO: 1, SEQ ID NO: 2, and SEQ ID NO: 3 are top candidates due to overall translational efficiency in K562 cells and selectivity of expression within K562 cells.

### Lipid Nanoparticle Formulation Testing for Oncoselectivity

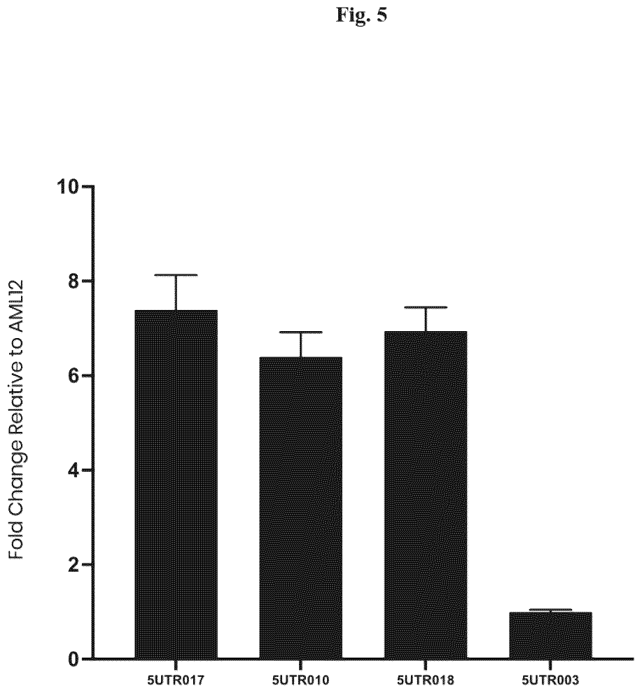

Lipid nanoparticles (LNPs) containing mRNA sequences translationally oncoselective were prepared to assess ability of mRNA elements to shift translational efficiency away from liver cells. Transfection of 3 independent replicates with 100 ng mRNA was performed using LNP. Selectivity was calculated by taking the ratio of the K562 cell line replicate over the BJ cell line average, then normalizing to the non-selective control (5′ UTR003). SEQ ID NO: 1 (5′ UTR017) is the top candidate based on the results. All mRNAs were formulated and transfected. Both data sets used luciferase as a reporter. Each bar represents the mean of three independent replicates error bars f standard deviation (SD). FIG. 5 shows SEQ ID NO: 1 (5′ UTR017) has about a 7-fold translational efficiency change relative to AML12 cell lines. Testing of other cancer cell populations (MC38, murine colorectal carcinoma cell line, and B16-F10, murine malignant melanoma cell line) with the LNP formulation of mRNA sequences was compared to a healthy murine liver cell population (AML12).

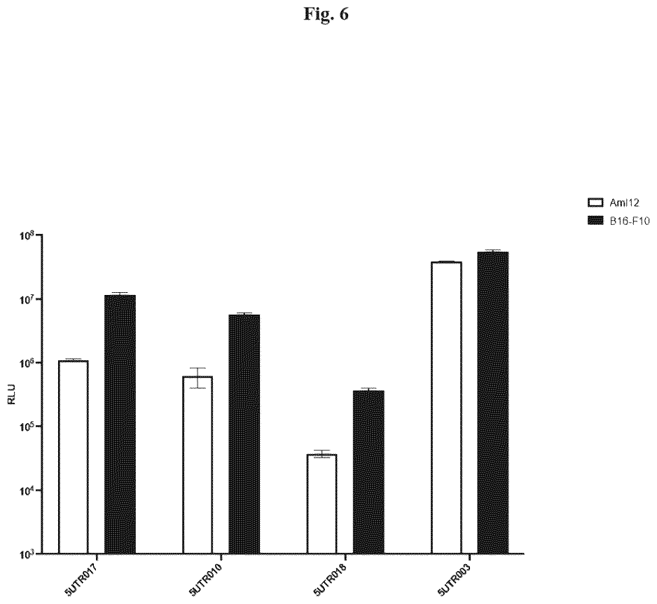

FIG. 6 shows raw nano-luciferase activity 48 hours following incubation with LNP-mRNA, showing increased translation in the cancer cell line B16-F10. FIG. 7 shows MC38 results compared with B16-F10 and AML12. mRNA derived from screening showed preferential translation of mRNA in two cancer cell lines. FIG. 8 shows B16-F10 cellular translation fold change relative to AML12 translation for tested RNA sequences.

### Example 2: Mouse and Human 3′ UTR155 and 3′ UTR152 Oncoselective Expression

Selective translation of SEQ ID NO: 4 (3′ UTR152) was investigated in more detail using bio-orthogonal methods. eGFP-myc-PEST was fused with the SEQ ID NO: 4 (3′ UTR152) to measure fluorescent intensity over time, giving a measurable product of the RNA translation.

### Methods

RNA was synthesized via T7 megascript kit and formulated with MessengerMax. Adherent cells were plated at 25,000 cells per well, suspension cells were plated at 50,000 cells per well, and cells were transfected at 0.1 μg/ml. The cells were then cultured for 144 hours and had fluorescence intensity measured for each well. Fluorescence intensity values were read with varioskan plate reader. Analysis done includes area under the curve (AUC) calculation by taking the integral of the fluorescent intensity over time curve. AUC calculations were normalized to BJ cells and then to 3′ UTR002 to get fold selectivity.

### Results

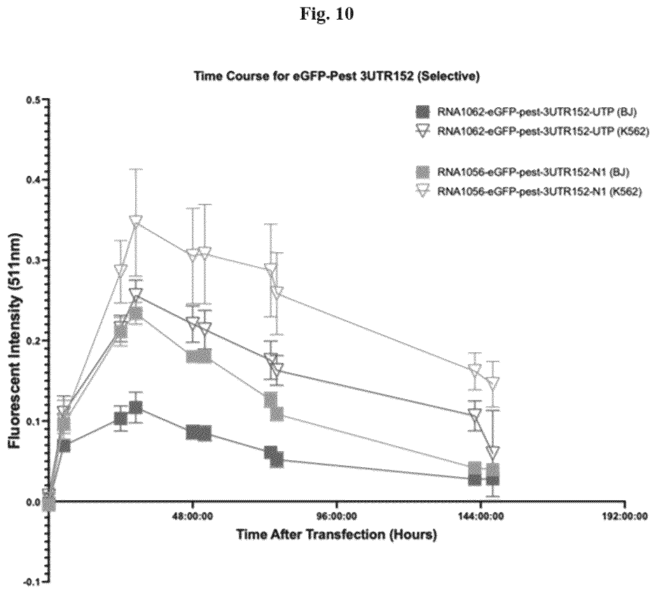

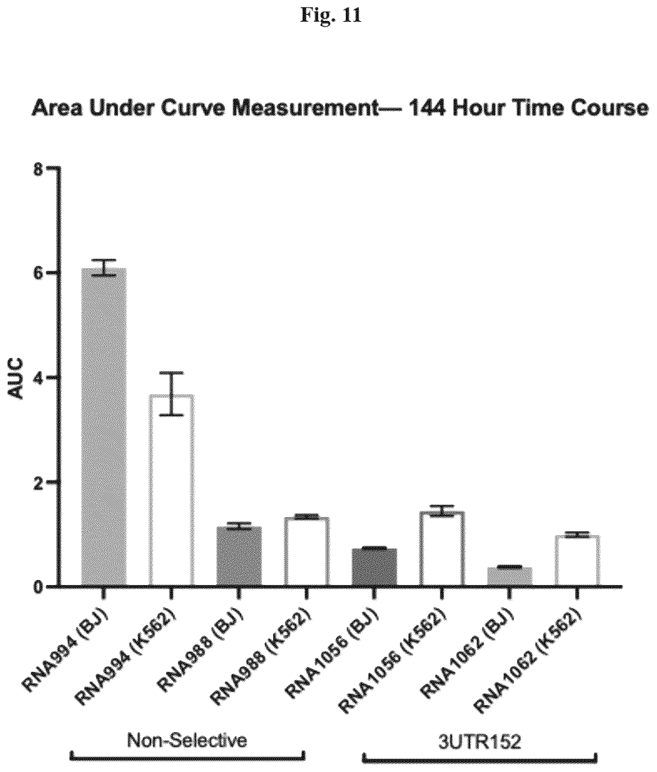

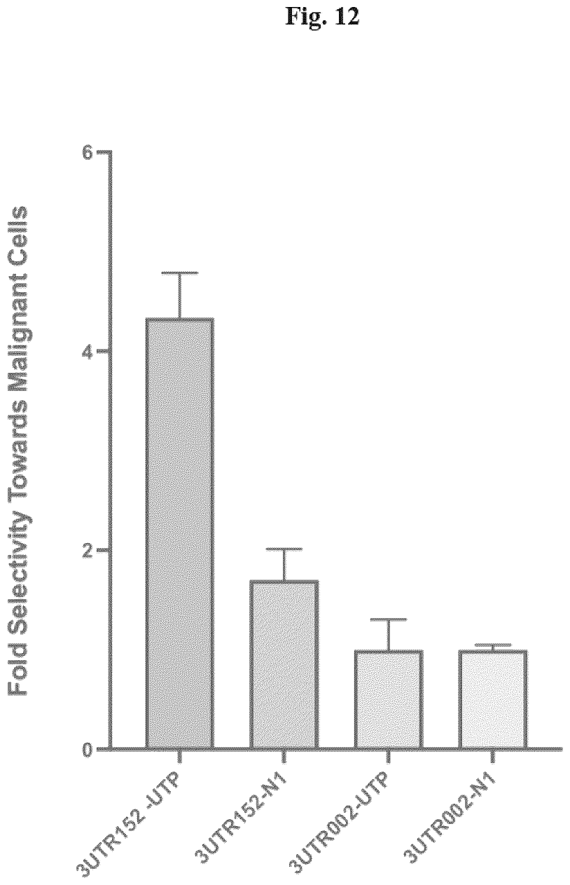

3′ UTR152 (SEQ ID NO: 4) showed differential translation in K562 over BJ cells using a different reporter protein. This outcome supports moving SEQ ID NO: 4 (3′ UTR152) sequence into further study. FIG. 9 shows resulting fluorescence intensity over time of a non-selective mRNA (3′ UTR002) sequence. FIG. 10 shows resulting fluorescence intensity over time of SEQ ID NO: 4 (3′ UTR152) mRNA sequence. FIG. 11 shows the resulting AUC measurement over 144 hours across healthy BJ cells and K562 cells between non-selective mRNA (3′ UTR002) and SEQ ID NO: 4 (5′ UTR152). FIG. 12 shows comparison of selective translation via fluorescence AUC over 144 hours of SEQ ID NO: 4 (3′ UTR152)-UTP and SEQ ID NO: 4 (3′ UTR152)-N1 methylpseudouridine versus 3′ UTR002-UTP and 3′ UTR002-N1 methylpseudouridine in BJ cells versus K562 cells. SEQ ID NO: 4 (3′ UTR152)-UTP demonstrated the greatest fold selectivity of translation towards malignant cells. The non-selective mRNA demonstrated the least fold selectivity for malignant cells.

### In Vivo Imaging

Selective translation of SEQ ID NO: 5 (3′ UTR155) was further tested via whole body IVIS imaging and ex vivo organ imaging.

### Methods

RNA was synthesized via T7 megascript kit and formulated using standard LNP protocol. Day 0 Inoculate B16-F10 Tumors near lower limbs to make clear distinction from liver. Inoculation comprised 3×10⁵/100 μl dosage subcutaneously. On day 9 animals were weighed, randomized, underwent tumor measurement, and experimental groups were dosed (5 animals per study group) with 5 μg of LNP-mRNA IV and 5 μg IT (10 ug total per mouse). On day 10 D-Luciferin was injected intraperitoneally. Measurement with IVIS was then performed. Assay was performed following 24 hours of incubation 5 μg of LNP-mRNA, Firefly Luciferase w/ D-luciferin was used to visualize translation.

### Results

A consistent region of interest was drawn around each organ. Pixel values were extracted in Perkin Elmer analysis software. FIG. 13 shows selective translation of Firefly Luciferase in B16-F10 cells versus liver cells (n=5, bars represent f standard error of mean (SEM), ***p≤0.001, lung data omitted). ANOVA was used to test significant change in fold selectivity against a non-selective RNA sequence. FIG. 14 shows visualization of fluorescence regions in oncoselective versus non-selective RNA sequences. Each organ was to liver and then normalized to 3′ UTR002 to calculate the relative fold selective translation. Results supported the finding RNA984, a N1-methylpseudouridine modified SEQ ID NO: 5 (3′ UTR152) was differentially translated at a higher level in tumor tissue compared to RNA1401 (3′ UTR002 N1 methylpseudouridine non-selective) mRNA.

### Additional Oncoselectivity Testing in Cancer Cells

Selective translation of SEQ ID NO: 4 was investigated in six cancer cell models relative to human primary hepatocytes. The cell line information is summarized in Table 1.

TABLE 1
Cell lines investigated for oncoselective SEQ ID NO: 4.

| Cell Line | Description |
|---|---|
| HsHEP | Single Donor, primary hepatocyte |
| K562 (Human) | lymphoblast cells isolated from the bone |
| | marrow of a 53-year-old chronic |
| | myelogenous leukemia patient |
| H1299 (Human) | epithelial-like cell that was isolated from |
| | the lung of a White, 43-year-old, male |
| | patient with carcinoma |
| SK-MEL | melanocytes isolated from the skin tissue |
| | of a 51-year-old, male patient with |
| | malignant melanoma |
| A549 | lung carcinoma epithelial cells |
| HCT116 | human colorectal carcinoma cell line |
| | initiated from an adult male |

### Method

RNA was synthesized via T7 megascript kit and formulated with MessengerMax. BJ cells were plated at 25,000 cells per well. K562 cells were plated at 50,000 cells per well. Cell lines were transfected at 0.1 μg/ml and cultured for 48 hours. Preparation of luminescence readout was performed with One Glo assay. Luminescence values were read with a varioskan plate reader. Each cell was normalized to human primary hepatocytes and then normalized to 3′ UTR002 to calculate the relative fold selective translation. Table 2 lists the SEQ ID of RNA tested for oncoselectivity within the cell lines shown in Table 1.

TABLE 2

| RNA ID | Nucleotide | 3′ UTR |
|---|---|---|
| RNA651-001 | Unmodified (UTP) | SEQ ID NO: 4 (3′ UTR152) |
| RNA984-001 | N1-Methylpseudouridine | SEQ ID NO: 4 (3′ UTR152) |
| RNA847-002 | Unmodified (UTP) | 3UTR002 |
| RNA1401-001 | N1-Methylpseudouridine | 3UTR002 |

### Results

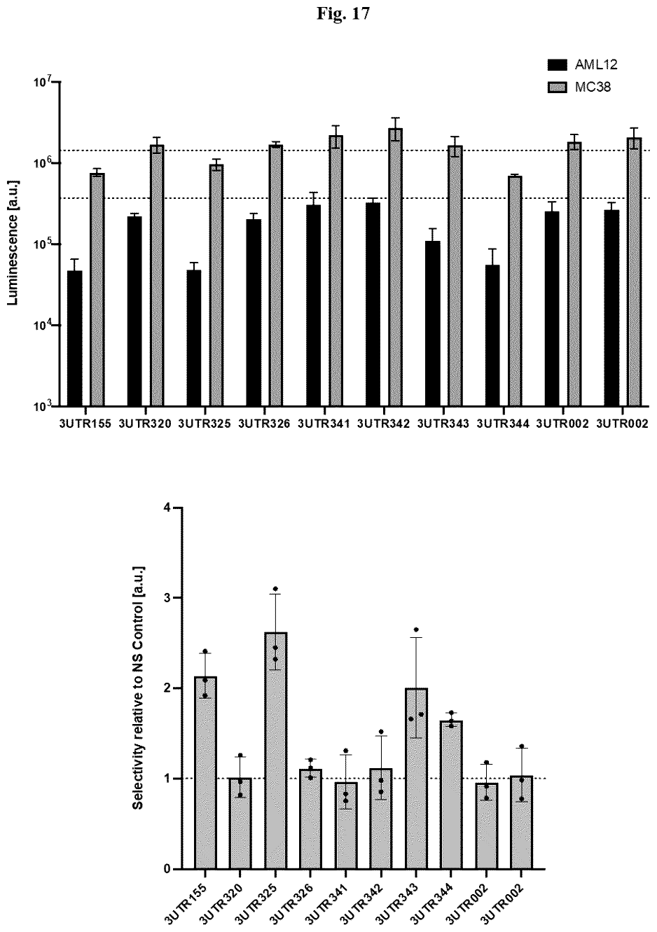

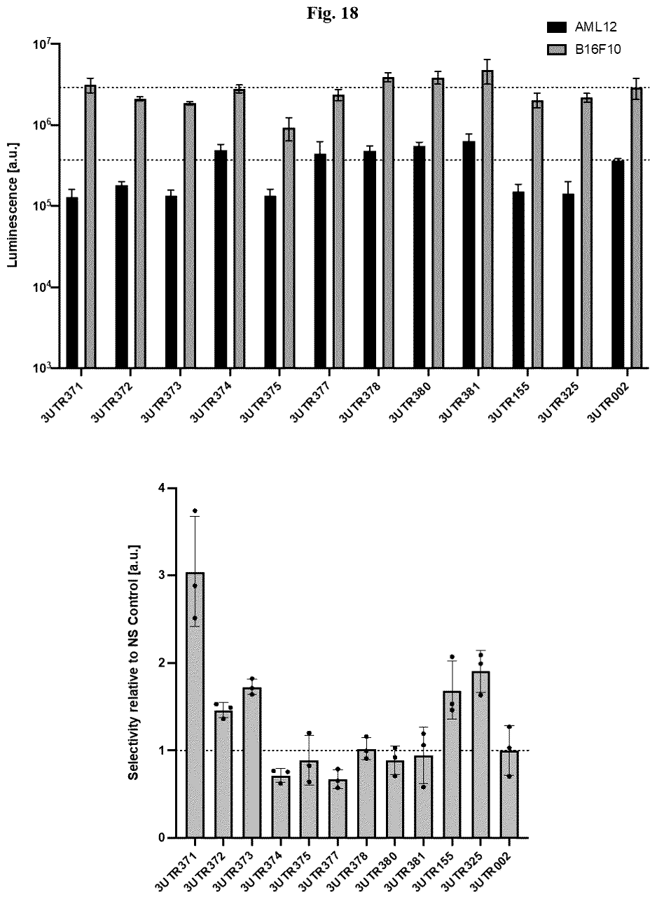

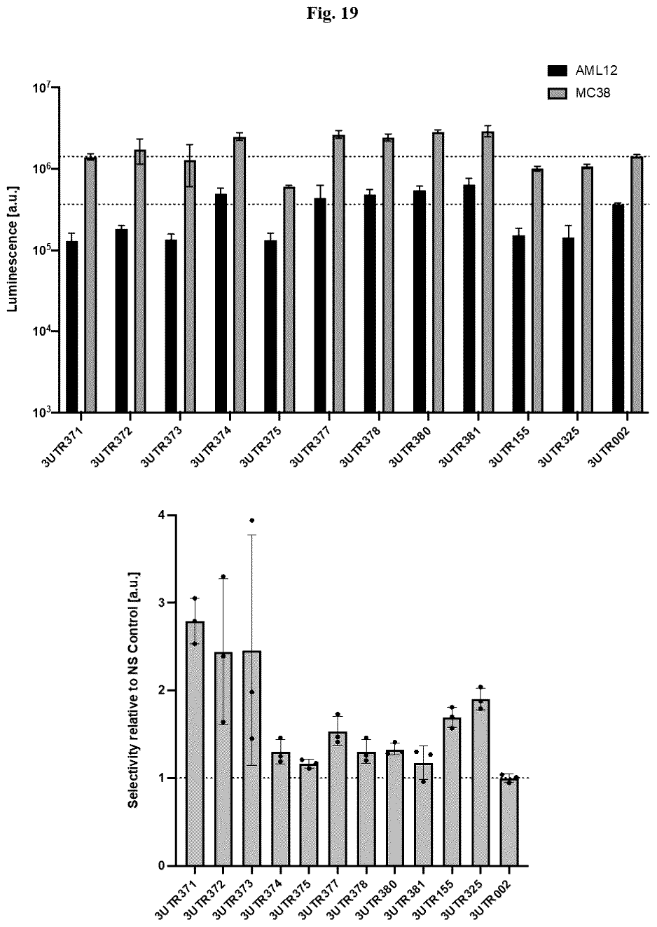

FIG. 15 shows selective translation in human cancer cell lines versus human primary hepatocytes (H-sHep) (n=6, bars represent f standard deviation, ordinary two-way ANOVA, ns p>0.05, *** p<0.001, ***p<0.001). The results suggested RNA984, an N1 methylpseudouridine SEQ ID) NO: 4 (3′ UTR152), was differentially translated at a higher rate in all cancer cell lines tested except A549 relative to 3′ UTR002. FIG. 16 shows the result of RNA translation measured by luminescence assay in B16-F10 and AML12 cells of truncated SEQ ID NO: 5 (3′ UTR155). FIG. 17 illustrates the result of RNA translation measured by luminescence assay in MC38 and AML12 cells of truncated SEQ ID NO: 5 (3′ UTR155). FIG. 18 illustrates the result of RNA translation measured by luminescence assay in B16-F10 and AML12 cells of truncated SEQ ID NO: 5 (3′ UTR155). FIG. 19 illustrates the result of RNA translation measured by luminescence assay in MC38 and AML12 cells of additional versions of truncated SEQ ID NO: 5 (3′ UTR155).

### Example 3. Identification of Additional 3′ UTR Targets for N1-Methylpseudouridylation

In order to test selectivity and translational, in vitro screening of mRNA elements to shift translation efficiency into malignant cells was assessed.

### Methods

A Firefly Luciferase Reporter was used to measure translation of mRNA sequences. Transfection of 3 independent replicates was achieved with 100 ng mRNA LNP formulation. Selectivity was calculated by taking the ratio of the K562, B16-F10, or MC38 replicate over the BJ or AML12 average, then normalizing to the non-selective control, 5′ UTR003 mRNA.

### Results

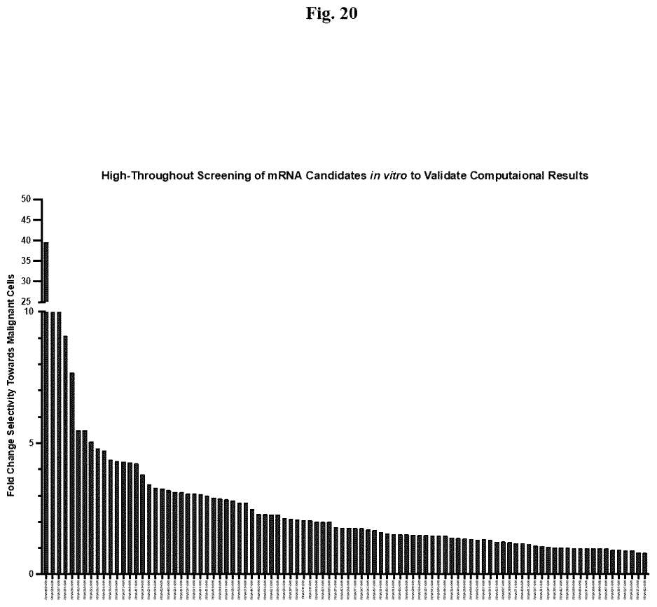

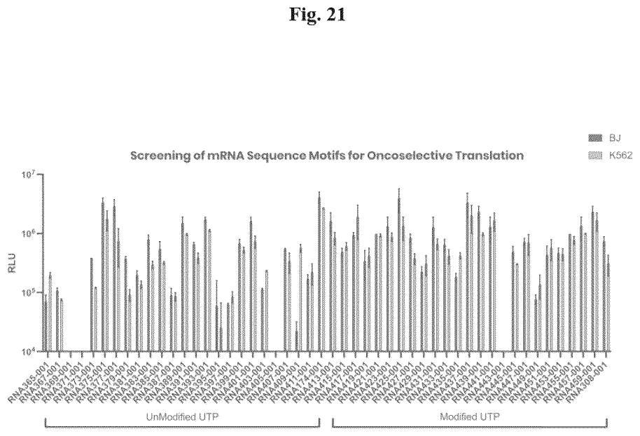

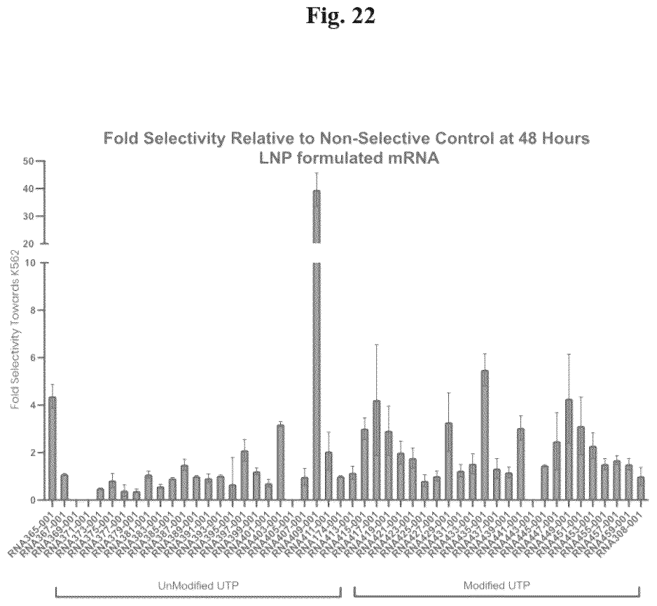

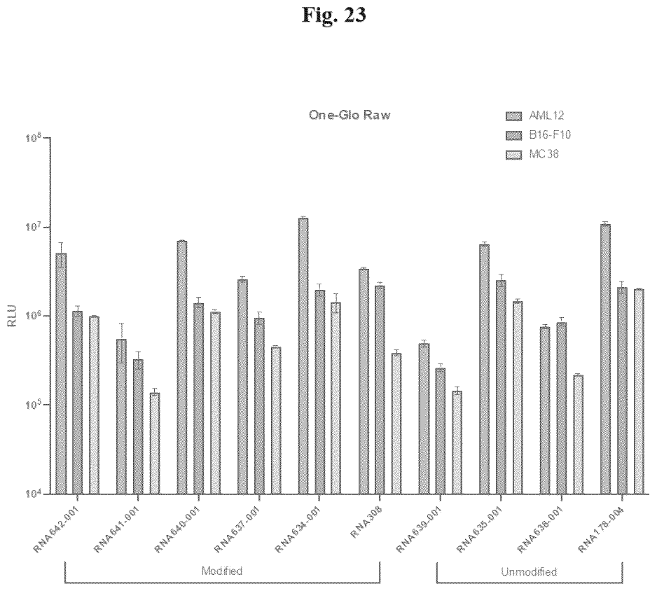

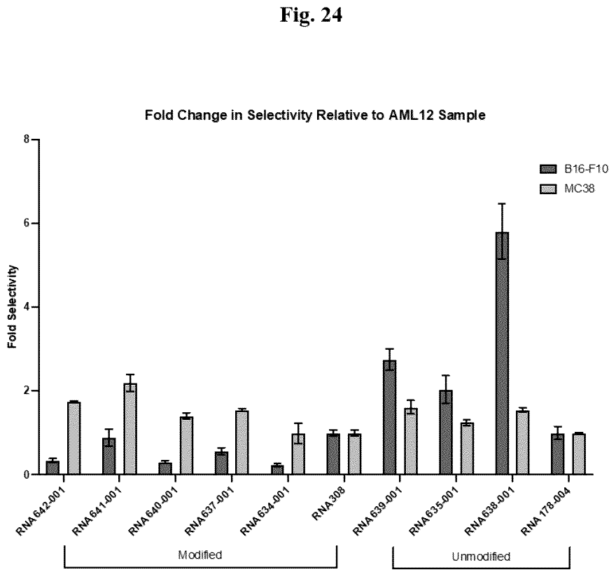

SEQ ID NO: 6 (3′ UTR047) was the top candidate screened from the library. FIG. 20 shows the computational results across the broader library of mRNA sequences tested. SEQ ID NO: 6 (3′ UTR047) showed an almost 40 fold translation selectivity for the cancer cell line K562, whereas FIG. 21 shows the screening of mRNA sequence motifs for oncoselective translation. FIG. 22 shows the screening results of mRNA sequence motifs for oncoselective translation, including across unmodified UTP and modified UTP. FIG. 23 shows the fold of translational selectivity of the library mRNA sequences tested, including modified UTP and unmodified UTP formulations. Further comparison is shown in FIG. 24, with the B16-F10 and MC-38 cell line translations compared with the healthy AML12 cell line. Several RNA sequences demonstrated different selectivity, particularly for the B16-F10 cell line.

### Example 4: Motif Attributes of Lead Oncoselective RNA Sequences

Sequence analysis of motifs of lead RNA candidates was performed to identify sequence motifs effective in oncoselectivity. Sequences were aligned and analyzed for patterns and similarity.

### Results

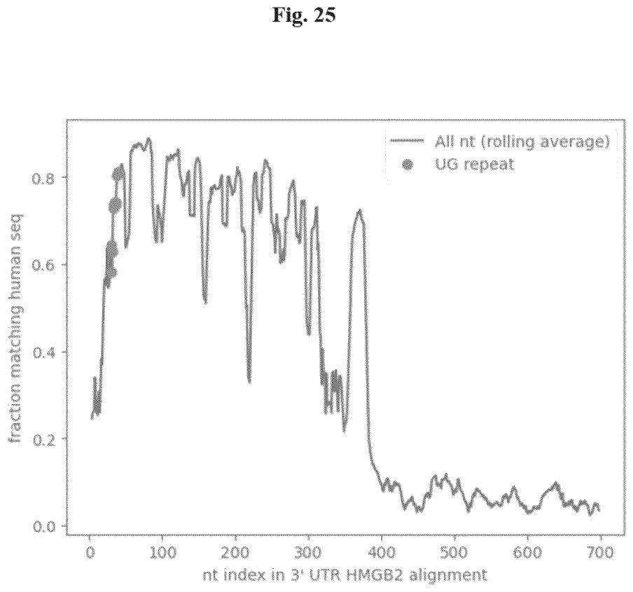

Sequence attributes for the HMGB2 3′ UTR RNA revealed nucleotide ratios of: Adenine: 30.4%, Cytosine: 11.7%, Guanine: 17.2%, Uracil: 40.7%, with an overall G-C content of 28.9%. The sequence attributes of 3′ UTR HMGB2 show a distinct sequence motif region. FIG. 25 shows the nucleotide index in 3′-UTR HMGB2 alignment versus the sequence fraction matching human sequences. A plot of evolutionary conservation of bases to human sequences in the functional region of the sequence shows conserved motif structure as seen in FIG. 26.

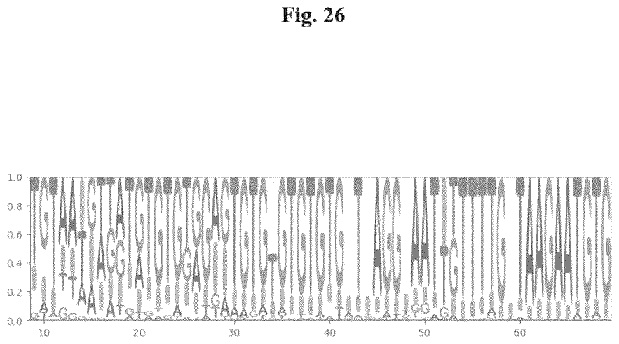

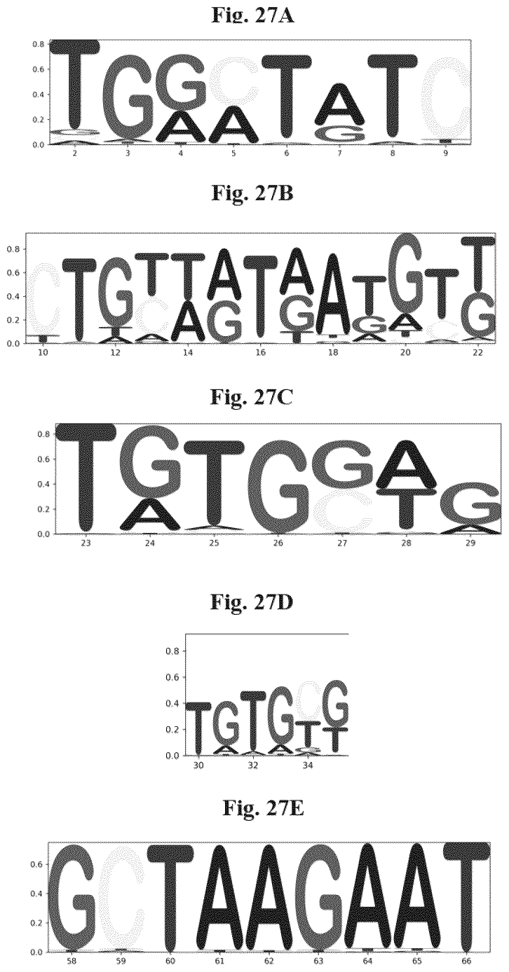

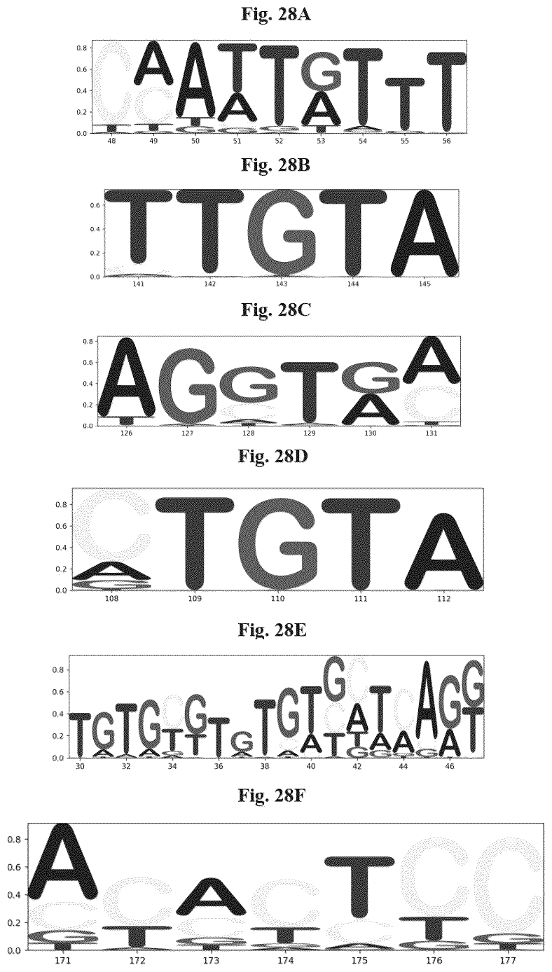

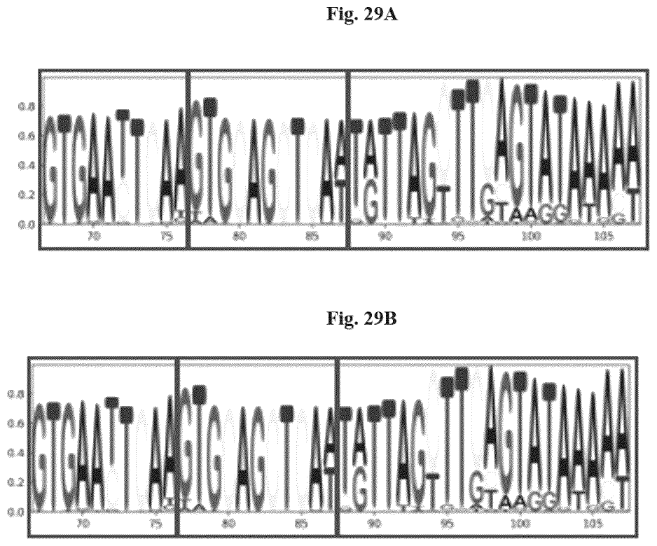

FIG. 27A, FIG. 27B, FIG. 27C, FIG. 27E, FIG. 28A, FIG. 28B, FIG. 28C, FIG. 28D, FIG. 28E, FIG. 28F, FIG. 29A and FIG. 29B show sequence elements of interest revealed upon analysis. Motifs and actual aligned sequences are shown in Table 3.

TABLE 3
Oncoselective motif sequence alignment

| 3' UTR | | |
|---|---|---|
| sequence | | |
| location | Actual sequence | Motif sequence |
| 2-9 | TGGCTATC | TGRMTNTC |
| | (SEQ ID NO: 700) | (SEQ ID NO: 720) |
| 10-22 | CTTTAATGATGCG | CTGYWRTRADGYK |
| | (SEQ ID NO: 701) | (SEQ ID NO: 721) |
| 23-29 | TGTGGAA | TGTGSWN |
| | (SEQ ID NO: 702) | (SEQ ID NO: 722) |
| 30-35 | TGTGTG | TRTGYG |
| | (SEQ ID NO: 703) | (SEQ ID NO: 723) |
| 30-47 | TGTGTGTGTGTGCTCAGG | TRTGYGTNTGTGCTCAGG |
| | (SEQ ID NO: 704) | (SEQ ID NO: 724) |
| 48-56 | CAATTATTT | CMAWTRTKT |
| | (SEQ ID NO: 705) | (SEQ ID NO: 725) |
| 58-66 | GCTAAGAAT | GCTAAGAAT |
| | (SEQ ID NO: 706) | (SEQ ID NO: 726) |
| 141-145 | YTGTA | CTGTA |
| | (SEQ ID NO: 707) | (SEQ ID NO: 727) |
| 126-131 | AGCTGA | AGVTRM |
| | (SEQ ID NO: 708) | (SEQ ID NO: 728) |
| 108-112 | CTGTA | CTGTA |
| | (SEQ ID NO: 709) | (SEQ ID NO: 729) |
| 171-177 | ACACUCC | AYNBUCC |
| | (SEQ ID NO: 710) | (SEQ ID NO: 730) |

Aligning a redundant part of the larger motif gives a smaller region of interest, GTGMABYYAA (SEQ ID NO: 161). These motifs provide further evidence of a link between oncoselectivity and specific 3′ UTR locations within tested RNA sequences.

### Example 5: Truncation of Oncoselective RNA Sequences

Improvement of the translation of 3′ UTR047 was targeted by truncating the sequence while retaining the selective translation.

### Methods

mRNA was synthesized via T7 megascript kit and formulated with messengermax. BJ cells were plated at 25,000 cells per well and K562 cells were plated at 50,000 cells per well. The transfection was performed at a concentration of 0.1 μg/ml and cultured for 48 hours. Luminesce values were determined by applying one glo and reading with a varioskan plate reader. Fold Selectivity was calculated by normalizing replicates to the average of BJ cells and then normalizing to a non-selective UTR (3′ UTR002). Fold enhancement in translation was determined by normalizing to 3′ UTR047.

### Results

SEQ ID NO: 7 (RNA887-3′ UTR117) retained the selectivity profile of parent SEQ ID NO: 6 (UTR047) while improving on the total protein output. The total protein output was not significantly different than the nonselective UTR002. FIG. 30 shows retention of selectivity in malignant cells based on RNA sequence (n=3, students t-test, ns=no significant change in selectivity vs parent UTR. Bars represent mean±standard deviation (SD)). FIG. 31 shows the fold enhancement in translational efficiency over parent UTR (n=3, students t-test, ns=no significant change in translational efficiency vs non-selective RNA sequence, bars represent mean±standard deviation (SD)).

### Screening of Top Candidates in AML12, B16-F10, and MC38 Mouse Cells

Top candidate sequences were tested in healthy AML12 cells versus cancerous cell lines including B16-F10 and MC38.

### Methods

RNA was synthesized via T7 megascript kit and formulated with standard LNP formulation. BJ cells were plated at 25,000 cells per well. K562 cells were plated at 50,000 cells per well. Cells were transfected at 0.1 μg/ml and cultured for 48 hours. Luminesce values were determined by applying one glo reagent from Promega and reading with varioskan plate reader. Fold selectivity was calculated by normalizing replicates to the average of BJ and then normalizing to 3′ UTR002 (Non-selective UTR).

### Results

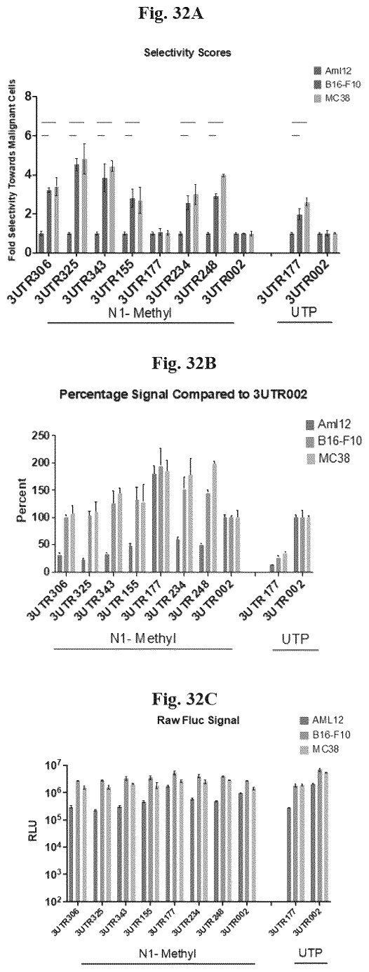

FIG. 32A shows the selectivity scores of mRNA sequences tested within the respective cell lines tested. An ANOVA test was run comparing the mean selectivity scores of each UTR for B16-F10 vs AML12 and MC38 vs AML12 (n=3, *p-value>0.05, ** p-value>0.01, *** p-value>0.001). FIG. 32B shows relative translation calculations, including percentage of Firefly luciferase (FLuc) signal relative to 3′-UTR002, the non-selective control sequence, of each mRNA sequence tested relative to 3′ UTR002. FIG. 32C shows the raw FLuc signal based on cell lines. The results demonstrated a selective expression in cancer cells.

### Example 6. Oncoselective Expression in Cancer Cells

Oncoselective expression in cancer cells was measured in two types of cancer cells (MC38 and B16F10). FIG. 34 illustrates oncoselective sequences (136-318 and 137-319) exhibiting increased selectivity of expression compared to control sequences. 325-343-488 denote oncoselective sequences based on truncations of sequences of untranslated regions. FIG. 34 further illustrates combination of various 5′ or 3′ untranslated region (UTR) to arrive at oncoselective sequences for increased selective expression in the cancer cells. Examples of the oncoselective sequences can be found in SEQ ID NOs: 101-146.

Oncoselective expression in cancer cells was measured in two types of cancer cells (MC38 and B16F10). FIG. 35A and FIG. 35B illustrate oncoselective sequences (136-318 and 137-319) exhibiting increased selectivity of expression compared to control sequences. 325-343-488 denote oncoselective sequences based on truncations of sequences of untranslated regions. FIG. 35A and FIG. 35B further illustrate combination of various 5′ or 3′ untranslated region (UTR) to arrive at oncoselective sequences for increased selective expression in the cancer cells. Examples of the oncoselective combination sequences can be found in SEQ ID NOs 101-146. As shown in FIG. 35A and FIG. 35B, the 3′-UTR sequence 3′ UTR155 (SEQ ID NO: 4) and truncation of 3′ UTR155 (e.g., 3′ UTR325) illustrated radiance and expression selectivity in MC38 tumor cell model. The 3′-UTR candidate (paired UTR) 5′ UTR137 (SEQ ID NO: 93) or 3′ UTR319 (SEQ ID NO: 97) illustrated radiance and expression selectivity in both B16F10 and MC38 tumor models.

FIG. 35C illustrates transfection (utilizing MessengerMAX transfection reagent) efficiency of the oncoselective sequences described herein (5UTR176 or SEQ ID NO: 140; 5UTR-177-5UTR182; or a combination of 5UTR025 or SEQ ID NO: 138 and 3UTR002 or SEQ ID NO: 94) as determined by luciferase relative light unit (RLU) in non-cancerous cell (human umbilical vein endothelial cell or HUVEC), myelogenous leukemia cell (K562), or non-small cell lung carcinoma cell (H1299). FIG. 35D illustrates the selectivity of the oncoselective sequences of FIG. 35C for driving expression of the luciferase in cancer cell compared to non-cancerous cell.

FIG. 35E illustrates replicate 1 of peripheral LNP transfection of the oncoselective sequences described herein (3UTR155 or SEQ ID NO: 123; 3UTR578 or SEQ ID NO: 124; 3UTR579 or SEQ ID NO: 125; 3UTR319 or SEQ ID NO: 121; 3UTR583 or SEQ ID NO: 129; 3UTR584 or SEQ ID NO: 130; 3UTR585 or SEQ ID NO: 131; combination of 5UTR136 or SEQ ID NO: 139 and 3UTR318 or SEQ ID NO: 122; combination of 5UTR136 or SEQ ID NO: 139 and 3UTR580 or SEQ ID NO: 126; or 3UTR002 or SEQ ID NO: 94) in HUVEC versus K562 (biological replicate 1 with n=3 technical replicates). FIG. 35F illustrates replicate 1 of the selectivity of the oncoselective sequences (as shown in FIG. 35E) described herein for driving expression of the luciferase in cancer cell compared to non-cancerous cell. FIG. 35G illustrates replicate 2 of peripheral LNP transfection of the oncoselective sequences described herein (3UTR155 or SEQ ID NO: 123; 3UTR578 or SEQ ID NO: 124; 3UTR579 or SEQ ID NO: 125; 3UTR319 or SEQ ID NO: 121; 3UTR583 or SEQ ID NO: 129; 3UTR584 or SEQ ID NO: 130; 3UTR585 or SEQ ID NO: 131; combination of 5UTR136 or SEQ ID NO: 139 and 3UTR318 or SEQ ID NO: 122; combination of 5UTR136 or SEQ ID NO: 139 and 3UTR580 or SEQ ID NO: 126; or 3UTR002 or SEQ ID NO: 94) in HUVEC versus K562 (biological replicate 1 with n=3 technical replicates). FIG. 35H illustrates replicate 2 of the selectivity of the oncoselective sequences (as shown in FIG. 35G) described herein for driving expression of the luciferase in cancer cell compared to non-cancerous cell. FIG. 35I illustrates combination of replicate 1 (FIG. 35E) and replicate 2 (FIG. 35G) of peripheral LNP transfection of the oncoselective sequences described herein in HUVEC versus K562 (biological replicate 1 with n=3 technical replicates). FIG. 35J illustrates combination of replicate 1 (FIG. 35F) and replicate 2 (FIG. 35H) of the selectivity of the oncoselective sequences described herein for driving expression of the luciferase in cancer cell compared to non-cancerous cell.

FIG. 35K illustrates replicate 1 of peripheral LNP transfection of the oncoselective sequences described herein (3UTR002 or SEQ ID NO: 94; 3UTR155 or SEQ ID NO: 123; 3UTR319 or SEQ ID NO: 121; 3UTR579 or SEQ ID NO: 125; 3UTR584 or SEQ ID NO: 130; 3UTR592 or SEQ ID NO: 132; 3UTR593 or SEQ ID NO: 133; 3UTR594 or SEQ ID NO: 135; 3UTR597 or SEQ ID NO: 137; 3UTR598 or SEQ ID NO: 134; or 3UTR596 or SEQ ID NO: 136) in HUVEC versus K562 (biological replicate 1 with n=3 technical replicates). FIG. 35L illustrates replicate 1 of the selectivity of the oncoselective sequences (as shown in FIG. 35K) described herein for driving expression of the luciferase in cancer cell compared to non-cancerous cell. FIG. 35M illustrates replicate 2 of peripheral LNP transfection of the oncoselective sequences described herein (3UTR002 or SEQ ID NO: 94; 3UTR155 or SEQ ID NO: 123; 3UTR319 or SEQ ID NO: 121; 3UTR579 or SEQ ID NO: 125; 3UTR584 or SEQ ID NO: 130; 3UTR592 or SEQ ID NO: 132; 3UTR593 or SEQ ID NO: 133; 3UTR594 or SEQ ID NO: 135; 3UTR597 or SEQ ID NO: 137; 3UTR598 or SEQ ID NO: 134; or 3UTR596 or SEQ ID NO: 136) in HUVEC versus K562 (biological replicate 1 with n=3 technical replicates). FIG. 35N illustrates replicate 2 of the selectivity of the oncoselective sequences (as shown in FIG. 35M) described herein for driving expression of the luciferase in cancer cell compared to non-cancerous cell. FIG. 35O illustrates combination of replicate 1 (FIG. 35K) and replicate 2 (FIG. 35M) of peripheral LNP transfection of the oncoselective sequences described herein in HUVEC versus K562 (biological replicate 1 with n=3 technical replicates). FIG. 35P illustrates combination of replicate 1 (FIG. 35L) and replicate 2 (FIG. 35N) of the selectivity of the oncoselective sequences described herein for driving expression of the luciferase in cancer cell compared to non-cancerous cell.

FIG. 35Q illustrates replicate 1 of peripheral LNP transfection of the oncoselective sequences described herein (3UTR002 or SEQ ID NO: 94; 3UTR155 or SEQ ID NO: 123; 3UTR319 or SEQ ID NO: 121; 3UTR579 or SEQ ID NO: 125; 3UTR584 or SEQ ID NO: 130; 3UTR592 or SEQ ID NO: 132; 3UTR593 or SEQ ID NO: 133; 3UTR594 or SEQ ID NO: 135; 3UTR597 or SEQ ID NO: 137; 3UTR598 or SEQ ID NO: 134; or 3UTR596 or SEQ ID NO: 136) herein in MC38 (colon adenocarcinoma cell) versus AML12 (non-cancerous hepatocyte cell) (biological replicate 1 with n=3 technical replicates). FIG. 35R illustrates replicate 1 of the selectivity of the oncoselective sequences (as shown in FIG. 35Q) described herein for driving expression of the luciferase in cancer cell compared to non-cancerous cell. FIG. 35S illustrates replicate 2 of peripheral LNP transfection of the oncoselective sequences described herein (3UTR002 or SEQ ID NO: 94; 3UTR155 or SEQ ID NO: 123; 3UTR319 or SEQ ID NO: 121; 3UTR579 or SEQ ID NO: 125; 3UTR584 or SEQ ID NO: 130; 3UTR592 or SEQ ID NO: 132; 3UTR593 or SEQ ID NO: 133; 3UTR594 or SEQ ID NO: 135; 3UTR597 or SEQ ID NO: 137, 3UTR598 or SEQ ID NO: 134; or 3UTR596 or SEQ ID NO: 136) in MC38 versus AML12 (biological replicate 1 with n=3 technical replicates). FIG. 35T illustrates replicate 2 of the selectivity of the oncoselective sequences (as shown in FIG. 35S) described herein for driving expression of the luciferase in cancer cell compared to non-cancerous cell. FIG. 35U illustrates combination of replicate 1 (FIG. 35Q) and replicate 2 (FIG. 35S) of peripheral LNP transfection of the oncoselective sequences described herein in HUVEC versus K562 (biological replicate 1 with n=3 technical replicates). FIG. 35V illustrates combination of replicate 1 (FIG. 35R) and replicate 2 (FIG. 35T) of the selectivity of the oncoselective sequences described herein for driving expression of the luciferase in cancer cell compared to non-cancerous cell.

### Example 7. Oncoselective mRNA for Treating Cancer

Therapeutic efficacy of the a cocktail therapeutic combination therapy was examined in various cancer models. The cocktail therapeutic was contacted with LNPs and subsequently administered to various cancer mouse models.

### LNP Formulation

mRNA or mixture of mRNA was formulated in LNPs containing a cationic ionizable lipid LIP003 (1-Octylnonyl 8-[(2-hydroxyethyl)[8-(nonyloxy)-8-oxooctyl]amino]octanoate), cholesterol, DSPC, and PEG2K-DMG at the molar ratios of 50:38.5:10:1.5. The ratio of ionizable amines to mRNA phosphates was 6. Lipids were dissolved in ethanol and mRNA was dissolved in 50 mM citrate buffer pH 5.0. The organic and aqueous phases were combined at a flow rate of 12 mL/min on a Precision Nanosystems NanoAssemblr at a ratio of 3:1 (aqueous: organic). The product was immediately diluted to 16.5% ethanol using MilliQ water. After sitting at room temperature for 1 h, the diluted product was dialyzed against a volume of 50 mM Tris 45 mM NaCl pH 7.4 which was 300-fold in excess. The dialyzed product was concentrated using Amicon Ultra 100 kDa centrifuge tubes and sterile filtered. mRNA concentration and encapsulation were assessed by the Invitrogen Ribogreen assay. Sucrose was added to the LNPs at the final concentration of 300 mM and LNPs were stored at −80° C. until use.

### ELISA

This study used ELISA kits to measure cytokine mRNA expression levels in vitro. B16-F10 cells were reverse transfected with 200 ng of mRNA/LNP and protein expression was measured 24 hours later. IL-2 (Invitrogen, BMS221INST) and IFN-α (Invitrogen, BMS6027) demonstrated comparable levels of expression, with IL-2 levels measuring 270±37 μg/ml for 200 ng, while the negative control had a level of 18.9 pg/ml. The fact that IL-2 was detected in the untransfected control was consistent with literature data indicating that B16-F10 cells had the ability to produce IL-2 themselves (PMID: 24179494). IFN-α levels were 228.5±25.5 pg/ml for 200 ng, while the negative control had a level of 0 μg/ml. IL-12 and 11-15 ELISA assays were conducted according to the manufacturer's instructions (Invitrogen, BMS6004, Invitrogen, 88-7215).

### IL-12 Cell Activity Assay Reagents

HEK-Blue IL-12 Reporter Cells, Invivogen Cat #hkb-IL-12

Quanti-Blue Solution, Invivogen cat #rep-qbs

DMEM, 4.5 g/L Glucose, 2 mM L-glutamine, 10% heat-inactivated FBS, 100U/ml Pen.Strep, 100 ug/ml Normocin

### Method

Dilutions of samples in media as well as recombinant mouse IL-12 at 2 ug/ml, as a positive control was prepared. To a flat-bottom 96-well plate, 20 ul of pre-diluted samples and 180 ul of HEK cells at 280,000 cells/ml was added and incubated at 37° C. overnight. Next day, Quanti-Blue (QB) solution was prepared by adding 1 ml of QB reagent and 1 ml of QB buffer to 98 ml of sterile water.

In a separate flat bottom 96-w-ell plate, 130 ul Quanti-Blue solution and 50 ul of culture supernatant from the HEK cell culture were added, incubated at 37° C., and read at OD650. Curves of OD650 and dilution factor of concertation were plotted.

### IL-12 ELISA Assay Reagents

Plates were coated with 100 ul coating anti-IL-12 antibody diluted in PBS and incubated over night at 4° C. Next day, the plates were washed 3 times with 200 ul/well wash-buffer, and the wells were blocked with blocking solution for 1 hour at room temp. The wells were then washed 3 times with wash buffer. IL-12 standard or diluted samples were added in blocking buffer, incubated for 2 hours at room temp, and then washed 4 times. 100 ul/well anti-IL-biotin detection antibody was added followed by incubation for 1 hour at room temp and washing for 3-4 times. 100 ul streptavidin-HRP was added followed by incubation for 30 minutes at room temperature and washing for 3-4 times. 100 ul/well Substrate solution was added for 10 minutes, and then 100 ul of Stop solution was added. The plate was read at OD450.

### Mouse Tumor Experiments

For the B16-F10 syngeneic murine tumor model, 6-10 week-old female C57BL/6 mice were purchased from Charles River (Wilmington, MA. USA) and housed in a pathogen free facility at CRADL in Cambridge, MA. The B61-F10 (melanoma) cell line was purchased from ATCC (Baltimore, MD) and grown in Dulbecco's Modified Eagle Medium (DMEM) with 10% Fetal Bovine Serum (FBS). A total of 2.5×10⁵ cells were implanted subcutaneously into the right flank of each mouse. Tumors were monitored by digital caliper measurements. Tumor volume (V_{T}) was calculated using the formula V_{T}=L×W²/2. Mice were randomized and placed on study groups based on tumor size. Tumor volumes were measured 2-3 times per week. Mice were dosed intratumorally a total of 6 times every 2-4 days over a duration of 2 weeks. Mice were euthanized when reaching the tumor burden of 2000 mm³ or any other humane endpoint as outlined in the IACUC protocol.

For tolerability studies female C57BL/6 mice were purchased from Charles River (Wilmington, MA. USA) and housed in a pathogen free facility at CRADL in Cambridge, MA. These mice were naïve (tumor free) and placed on study after the acclimation period. The mice were given 4 doses every 3-4 days over a period of 10 days. Body weights were measured as needed. Groups were taken down at scheduled timepoints, harvesting whole blood, serum, and various organs for analysis. 13 organs (femur, ovaries, colon, spleen, kidneys, small intestine, liver, lungs, heart, stomach, brain, muscle, & skin) were collected and stored in 10% formalin for 24 hours at room temperature. The organs were then transferred to 70% ethanol and stored at 4° C. or sent for analysis. Whole blood & serum were sent to IDEXX Laboratories (North Grafton, MA) for analysis. Liver, spleen, & femurs were sent Histowiz (Long Island, NY) for immunohistochemistry.

MC38 syngeneic murine colon tumor model. The aim of the studies was two-fold: firstly, to evaluate the effectiveness of cocktail therapeutic and secondly, to compare the efficacy of cocktail therapeutic to that of its individual components. Animals were implanted with 3×10⁵ cells per 100 μl per mouse. The initial tumor volume averaged at 76.2 mm³ (ranging from 37.1 to 156.5 mm³). Ten days later the dosing started, mice received six intratumoral injections administered every other day. By day 40, the results indicated that IL-2 resulted in 4 complete responses (CRs), IL-12 resulted in 2 CRs, IL-15 resulted in 3 CRs, while IFN-α and the untreated control resulted in 0 CRs.

12-week-old female C57BL/6 mice were purchased from Charles River (Wilmington, MA. USA) and housed in a pathogen free facility at CRADL in Cambridge, MA. The MC38.k (colorectal) cell line was purchased from Kerafast (Boston, MA) and grown in Dulbecco's Modified Eagle Medium (DMEM)+Glutamine with 10% Fetal Bovine Serum (FBS). A total of 3×10⁵ cells were implanted subcutaneously into the right flank of each mouse. Tumors were monitored by digital caliper measurements. Tumor volume (V_{T}) was calculated using the formula V_{T}=L×W²/2. Mice were randomized and placed on study 10 days post implant with an average tumor burden of 128 mm³. Tumor volumes were measured 2-3 times per week. Mice were dosed intratumorally a total of 6 times every 2-3 days over a duration of 2 weeks. Mice were euthanized when reaching the tumor burden of 2000 mm³ or any other humane endpoint as outlined in the IACUC protocol. Mice were treated with payload encoded by nuclei acid sequences in Table 4 for a therapeutic as illustrated in Table 13.

TABLE 4
Nucleic acid sequence encoding payload

| | | | | SEQ |
|---|---|---|---|---|
| | Protein | | | NO |
| Payload | number | RNA# | Seq | NO |
| mtIL-12 | Pro006 | | AGGGUUUCGUCUUUAGAUCUUCUGGUCCCCACAGACUCAGAGAGAACCCGCCACCAUGUGC | 461 |
| | | | CCCCAGAAGCUGACCAUCAGCUGGUUCGCCAUCGUGCUGCUGGUGAGCCCUCUGAUGGCCA | |
| | | | UGUGGGAGCUCGAGAAGGACGUGUACGUGGUGGAGGUGGACUGGACCCCCGACGCCCCCGG | |
| | | | CGAGACCGUGAACCUGACCUGUGACACCCCCGAGGAGGACGACAUCACCUGGACCAGCGAC | |
| | | | CAGCGGCACGGCGUGAUCGGAAGCGGCAAGACCCUGACCAUCACCGUCAAGGAGUUCCUGG | |
| | | | ACGCCGGCCAGUACACCUGCCACAAGGGCGGCGAGACACUGAGCCACAGCCACCUGCUGUU | |
| | | | ACACAAGAAGGAGAACGGCAUCUGGAGCACCGAGAUUCUGAAGAACUUCAAGAACAAGACC | |
| | | | UUCCUGAAGUGCGAAGCCCCCAAUUACAGCGGCCGGUUCACCUGCAGCUGGCUGGUGCAGC | |
| | | | GGAACAUGGACCUGAAGUUCAAUAUCAAGUCAAGCAGCAGCAGCCCCGAUAGCCGGGCCGU | |
| | | | GACCUGCGGCAUGGCCAGCCUGAGCGCCGAGAAGGUGACCCUGGACCAGAGGGACUACGAG | |
| | | | AAGUACAGCGUGAGCUGUCAGGAGGACGUGACAUGCCCAACCGCCGAGGAGACCCUGCCCA | |
| | | | UCGAGCUGGCCCUGGAGGCCCGGCAGCAGAAUAAGUACGAGAAUUACAGCACCAGCUUCUU | |
| | | | CAUCCGGGACAUCAUCAAGCCCGACCCCCCUAAGAACCUGCAGAUGAAGCCCCUGAAGAAU | |
| | | | AGCCAGGUCGAGGUGAGCUGGGAGUACCCCGAUAGCUGGAGCACCCCCCACAGCUACUUCA | |
| | | | GCCUGAAGUUCUUCGUGCGGAUCCAGCGGAAGAAGGAGAAGAUGAAGGAGACCGAGGAGGG | |
| | | | CUGCAAUCAGAAGGGCGCAUUCCUGGUGGAGAAGACCAGCACCGAGGUGCAGUGCAAGGGC | |
| | | | GGCAACGUGUGCGUGCAGGCCCAGGAUCGGUACUACAACAGCAGCUGCAGCAAGUGGGCCU | |
| | | | GCGUGCCAUGCCGGGUGCGGAGCGGCGGCGGAGGCUCAGGUGGCGGCGGCUCAGGCGGGGG | |
| | | | CGGCAGCCGGGUGAUCCCCGUGAGCGGCCCCGCCCGGUGCCUUAGCCAGAGCCGGAAUCUG | |
| | | | CUGAAGACCACCGACGACAUGGUGAAGACCGCCCGGGAGAAGCUGAAGCACUACAGCUGCA | |
| | | | CCGCCGAGGACAUCGACCACGAGGACAUCACCCGGGACCAGACCAGCACCCUGAAGACCUG | |
| | | | CCUGCCCCUGGAGCUGCACAAGAACGAGAGCUGCCUGGCAACCCGGGAGACCAGCAGCACC | |
| | | | ACCCGGGGCAGCUGCCUCCCCCCCCAGAAGACCAGCCUGAUGAUGACCCUGUGCCUGGGGA | |
| | | | GCAUCUACGAGGACCUGAAGAUGUACCAGACCGAGUUCCAGGCUAUAAACGCCGCCCUGCA | |
| | | | GAACCACAACCACCAGCAGAUCAUCCUGGACAAGGGCAUGCUGGUGGCCAUCGACGAGCUG | |
| | | | AUGCAGAGCCUGAACCACAACGGCGAGACUCUGCGGCAGAAGCCCCCUGUGGGCGAGGCCG | |
| | | | AUCCCUACCGGGUGAAGAUGAAGCUGUGCAUCCUGCUGCACGCCUUCAGCACCCGGGUGGU | |
| | | | GACCAUCAACAGGGUGAUGGGCUACCUGAGCAGCGCUAGCGGCGGAGGUAGCGGAGGGGGC | |
| | | | GGCUCCGGCGGCGGCGGCAGCGGUGGAGGCGGCAGCGGCGGCGGUAGCCUGCAAGUGGUGA | |
| | | | UCAGCGCCAUCCUGGCCCUGGUGGUGCUGACCGUGAUCAGCCUGAUCAUCCUGAUCGGCGG | |
| | | | UGGCGGGAGCGGCAAGCCCAUCCCCAACCCACUGCUGGGCCUGGACAGCACCUGACUAACU | |
| | | | AAUUAAGCUGCCUUCUGCGGGGCUUGCCUUCUGGCCAUGCCCUUCUUCUCUCCCUUGCACC | |
| | | | UGUACCUCUUGGUCUUUGAAUAAAGCCUGAGUAGGAAGAAGCCUGCAUGCCUGGUUCUCUG | |
| | | | CGUCUGCGAAUUCGAUAUCCAGCGGCCGCGCUAGCGCUGAAAAAAAAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAAAAAAAAAAAAAAAAAAAA | |
| mtIL-12 | Pro006 | RNA312 | AGGGUUUCGUCUUUAGAUCUUCUGGUCCCCACAGACUCAGAGAGAACCCGCCACCAUGUGC | 462 |
| | | | CCCCAGAAGCUGACCAUCAGCUGGUUCGCCAUCGUGCUGCUGGUGAGCCCUCUGAUGGCCA | |
| | | | UGUGGGAGCUCGAGAAGGACGUGUACGUGGUGGAGGUGGACUGGACCCCCGACGCCCCCGG | |
| | | | CGAGACCGUGAACCUGACCUGUGACACCCCCGAGGAGGACGACAUCACCUGGACCAGCGAC | |
| | | | CAGCGGCACGGCGUGAUCGGAAGCGGCAAGACCCUGACCAUCACCGUCAAGGAGUUCCUGG | |
| | | | ACGCCGGCCAGUACACCUGCCACAAGGGCGGCGAGACACUGAGCCACAGCCACCUGCUGUU | |
| | | | ACACAAGAAGGAGAACGGCAUCUGGAGCACCGAGAUUCUGAAGAACUUCAAGAACAAGACC | |
| | | | UUCCUGAAGUGCGAAGCCCCCAAUUACAGCGGCCGGUUCACCUGCAGCUGGCUGGUGCAGC | |
| | | | GGAACAUGGACCUGAAGUUCAAUAUCAAGUCAAGCAGCAGCAGCCCCGAUAGCCGGGCCGU | |
| | | | GACCUGCGGCAUGGCCAGCCUGAGCGCCGAGAAGGUGACCCUGGACCAGAGGGACUACGAG | |
| | | | AAGUACAGCGUGAGCUGUCAGGAGGACGUGACAUGCCCAACCGCCGAGGAGACCCUGCCCA | |
| | | | UCGAGCUGGCCCUGGAGGCCCGGCAGCAGAAUAAGUACGAGAAUUACAGCACCAGCUUCUU | |
| | | | CAUCCGGGACAUCAUCAAGCCCGACCCCCCUAAGAACCUGCAGAUGAAGCCCCUGAAGAAU | |
| | | | AGCCAGGUCGAGGUGAGCUGGGAGUACCCCGAUAGCUGGAGCACCCCCCACAGCUACUUCA | |
| | | | GCCUGAAGUUCUUCGUGCGGAUCCAGCGGAAGAAGGAGAAGAUGAAGGAGACCGAGGAGGG | |
| | | | CUGCAAUCAGAAGGGCGCAUUCCUGGUGGAGAAGACCAGCACCGAGGUGCAGUGCAAGGGC | |
| | | | GGCAACGUGUGCGUGCAGGCCCAGGAUCGGUACUACAACAGCAGCUGCAGCAAGUGGGCCU | |
| | | | GCGUGCCAUGCCGGGUGCGGAGCGGCGGCGGAGGCUCAGGUGGCGGCGGCUCAGGCGGGGG | |
| | | | CGGCAGCCGGGUGAUCCCCGUGAGCGGCCCCGCCCGGUGCCUUAGCCAGAGCCGGAAUCUG | |
| | | | CUGAAGACCACCGACGACAUGGUGAAGACCGCCCGGGAGAAGCUGAAGCACUACAGCUGCA | |
| | | | CCGCCGAGGACAUCGACCACGAGGACAUCACCCGGGACCAGACCAGCACCCUGAAGACCUG | |
| | | | CCUGCCCCUGGAGCUGCACAAGAACGAGAGCUGCCUGGCAACCCGGGAGACCAGCAGCACC | |
| | | | ACCCGGGGCAGCUGCCUCCCCCCCCAGAAGACCAGCCUGAUGAUGACCCUGUGCCUGGGGA | |
| | | | GCAUCUACGAGGACCUGAAGAUGUACCAGACCGAGUUCCAGGCUAUAAACGCCGCCCUGCA | |
| | | | GAACCACAACCACCAGCAGAUCAUCCUGGACAAGGGCAUGCUGGUGGCCAUCGACGAGCUG | |
| | | | AUGCAGAGCCUGAACCACAACGGCGAGACUCUGCGGCAGAAGCCCCCUGUGGGCGAGGCCG | |
| | | | AUCCCUACCGGGUGAAGAUGAAGCUGUGCAUCCUGCUGCACGCCUUCAGCACCCGGGUGGU | |
| | | | GACCAUCAACAGGGUGAUGGGCUACCUGAGCAGCGCUAGCGGCGGAGGUAGCGGAGGGGGC | |
| | | | GGCUCCGGCGGCGGCGGCAGCGGUGGAGGCGGCAGCGGCGGCGGUAGCCUGCAAGUGGUGA | |
| | | | UCAGCGCCAUCCUGGCCCUGGUGGUGCUGACCGUGAUCAGCCUGAUCAUCCUGAUCGGCGG | |
| | | | UGGCGGGAGCGGCAAGCCCAUCCCCAACCCACUGCUGGGCCUGGACAGCACCUGACUAACU | |
| | | | AAACCGGUGGACACAUGGUUACCACCUGGCCCUGAGUGCAGUCAGACUGGGACAAAGGAGC | |
| | | | CGUGAAACACGCAAGGAGCUUCUGGCUUCUCAGCUUUGAGACCUGGGCUCUUUGGAGCACA | |
| | | | GAGAACUUUGAUUUUUAAGCUUCUACAGAUAUGGUCCUAGAGACUGGGGUCCUCAGACACU | |
| | | | CCCGCUGUAUGUGGUGAGCUUGGCUGGAAACUUGCCAUAGCCAGGACUGAGGGGCUGGCCC | |
| | | | CAGGACGCUCUCAAGAAAUGGGGCUACUGUGGUUUAGACACUCCUGCUGCUCACAUUGAUG | |
| | | | GGUGGCUAUUAAAGGCUAGCGAAUUCGAUAUCCAGCGGCCGCAAAAAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAAAAAAAAAAAAAAAAAAAAAAA | |
| IL-15 | Pro011 | RNA265 | AGGGUUUCGUCUUUAGAUCUUCUGGUCCCCACAGACUCAGAGAGAACCCGCCACCAUGGCU | 463 |
| | | | AGCCCCCAGCUGCGGGGCUACGGCGUGCAGGCCAUCCCUGUGCUGCUGUUACUGCUGCUGC | |
| | | | UCCUGCUGCUGCCCCUGCGGGUGACCCCCGGCACCACCUGCCCCCCCCCCGUGAGCAUCGA | |
| | | | GCACGCCGACAUCCGGGUGAAGAACUACAGCGUGAACAGCCGGGAGAGGUACGUGUGCAAU | |
| | | | AGCGGCUUCAAGCGGAAGGCCGGCACCAGCACCCUGAUCGAGUGCGUGAUCAACAAGAACA | |
| | | | CCAACGUGGCCCACUGGACCACCCCCAGCCUGAAGUGCAUCCGGGACCCCAGCUUAGCCCA | |
| | | | CUACAGCCCCGUGCCCACCGUGGUGACCCCAAAGGUGACCAGCCAGCCCGAGAGCCCCAGC | |
| | | | CCCAGCGCCAAGGAGCCUGAGGCCUUCUCCCCAAAGAGCGACACCGCCAUGACCACCGAGA | |
| | | | CCGCCAUCAUGCCCGGCAGCCGGCUGACCCCCAGCCAGACCACCAGCGCCGGCACCACAGG | |
| | | | CACCGGCAGCCACAAGAGCAGCCGGGCACCCAGCCUGGCCGCCACCAUGACCCUGGAGCCU | |
| | | | ACCGCUAGCACCAGCCUGCGGAUCACCGAGAUCAGCCCCCACAGCAGCAAGAUGACCAAGG | |
| | | | UGGCCAUCAGCACCAGCGUGCUGCUGGUGGGCGCCGGCGUGGUGAUGGCUUUCCUGGCCUG | |
| | | | GUACAUCAAGAGCCGGCAGCCCAGCCAGCCUUGCCGGGUGGAGGUGGAGACCAUGGAGACU | |
| | | | GUGCCCAUGACCGUGCGGGCCAGCAGCAAGGAGGACGAGGACACCGGCGCCGGGGGCAGCG | |
| | | | GCGGUAGCGGAGGCAGCGGCGGCAGCGGCGGAAGCGGCGGCUCCGGCGGCAUGAAGAUCCU | |
| | | | GAAGCCCUACAUGCGGAACACCAGCAUCAGCUGCUACCUGUGCUUCCUGCUGAACAGCCAC | |
| | | | UUCCUGACCGAGGCCGGCAUCCACGUGUUCAUCCUGGGCUGCGUGAGCGUGGGCCUGCCUA | |
| | | | AGACCGAGGCCAACUGGAUCGACGUGCGGUACGACCUGGAGAAGAUCGAGAGCCUGAUCCA | |
| | | | GAGCAUCCACAUCGACACCACCCUGUACACCGACAGCGACUUCCACCCCAGCUGCAAGGUG | |
| | | | ACCGCCAUGAACUGCUUUCUGCUGGAGCUGCAGGUGAUCCUGCACGAGUACAGCAACAUGA | |
| | | | CCCUGAACGAGACCGUGCGGAACGUGCUGUACCUGGCCAACAGCACCCUGAGCAGCAACAA | |
| | | | GAACGUGGCCGAGAGCGGCUGCAAGGAGUGCGAGGAGCUGGAGGAGAAGACCUUCACCGAG | |
| | | | UUCCUGCAGAGCUUCAUCCGGAUCGUGCAGAUGUUCAUCAACACCAGCUGACUAACUAAUU | |
| | | | AAGCUGCCUUCUGCGGGGCUUGCCUUCUGGCCAUGCCCUUCUUCUCUCCCUUGCACCUGUA | |
| | | | CCUCUUGGUCUUUGAAUAAAGCCUGAGUAGGAAGAAGCCUGCAUGCCUGGUUCUCUGCGUC | |
| | | | UGCGAAUUCGAUAUCCAGCGGCCGCGCUAGCGCUGAAAAAAAAAAAAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAAAAAAAAAAAAAAAA | |
| IL-15 | Pro011 | RNA854 | AGGGUUUCGUCUUUAGAUCUUCUGGUCCCCACAGACUCAGAGAGAACCCGCCACCAUGGCU | 464 |
| | | | AGCCCCCAGCUGCGGGGCUACGGCGUGCAGGCCAUCCCUGUGCUGCUGUUACUGCUGCUGC | |
| | | | UCCUGCUGCUGCCCCUGCGGGUGACCCCCGGCACCACCUGCCCCCCCCCCGUGAGCAUCGA | |
| | | | GCACGCCGACAUCCGGGUGAAGAACUACAGCGUGAACAGCCGGGAGAGGUACGUGUGCAAU | |
| | | | AGCGGCUUCAAGCGGAAGGCCGGCACCAGCACCCUGAUCGAGUGCGUGAUCAACAAGAACA | |
| | | | CCAACGUGGCCCACUGGACCACCCCCAGCCUGAAGUGCAUCCGGGACCCCAGCUUAGCCCA | |
| | | | CUACAGCCCCGUGCCCACCGUGGUGACCCCAAAGGUGACCAGCCAGCCCGAGAGCCCCAGC | |
| | | | CCCAGCGCCAAGGAGCCUGAGGCCUUCUCCCCAAAGAGCGACACCGCCAUGACCACCGAGA | |
| | | | CCGCCAUCAUGCCCGGCAGCCGGCUGACCCCCAGCCAGACCACCAGCGCCGGCACCACAGG | |
| | | | CACCGGCAGCCACAAGAGCAGCCGGGCACCCAGCCUGGCCGCCACCAUGACCCUGGAGCCU | |
| | | | ACCGCUAGCACCAGCCUGCGGAUCACCGAGAUCAGCCCCCACAGCAGCAAGAUGACCAAGG | |
| | | | UGGCCAUCAGCACCAGCGUGCUGCUGGUGGGCGCCGGCGUGGUGAUGGCUUUCCUGGCCUG | |
| | | | GUACAUCAAGAGCCGGCAGCCCAGCCAGCCUUGCCGGGUGGAGGUGGAGACCAUGGAGACU | |
| | | | GUGCCCAUGACCGUGCGGGCCAGCAGCAAGGAGGACGAGGACACCGGCGCCGGGGGCAGCG | |
| | | | GCGGUAGCGGAGGCAGCGGCGGCAGCGGCGGAAGCGGCGGCUCCGGCGGCAUGAAGAUCCU | |
| | | | GAAGCCCUACAUGCGGAACACCAGCAUCAGCUGCUACCUGUGCUUCCUGCUGAACAGCCAC | |
| | | | UUCCUGACCGAGGCCGGCAUCCACGUGUUCAUCCUGGGCUGCGUGAGCGUGGGCCUGCCUA | |
| | | | AGACCGAGGCCAACUGGAUCGACGUGCGGUACGACCUGGAGAAGAUCGAGAGCCUGAUCCA | |
| | | | GAGCAUCCACAUCGACACCACCCUGUACACCGACAGCGACUUCCACCCCAGCUGCAAGGUG | |
| | | | ACCGCCAUGAACUGCUUUCUGCUGGAGCUGCAGGUGAUCCUGCACGAGUACAGCAACAUGA | |
| | | | CCCUGAACGAGACCGUGCGGAACGUGCUGUACCUGGCCAACAGCACCCUGAGCAGCAACAA | |
| | | | GAACGUGGCCGAGAGCGGCUGCAAGGAGUGCGAGGAGCUGGAGGAGAAGACCUUCACCGAG | |
| | | | UUCCUGCAGAGCUUCAUCCGGAUCGUGCAGAUGUUCAUCAACACCAGCUGACUAACUAAAC | |
| | | | CGGUGGACACAUGGUUACCACCUGGCCCUGAGUGCAGUCAGACUGGGACAAAGGAGCCGUG | |
| | | | AAACACGCAAGGAGCUUCUGGCUUCUCAGCUUUGAGACCUGGGCUCUUUGGAGCACAGAGA | |
| | | | ACUUUGAUUUUUAAGCUUCUACAGAUAUGGUCCUAGAGACUGGGGUCCUCAGACACUCCCG | |
| | | | CUGUAUGUGGUGAGCUUGGCUGGAAACUUGCCAUAGCCAGGACUGAGGGGCUGGCCCCAGG | |
| | | | ACGCUCUCAAGAAAUGGGGCUACUGUGGUUUAGACACUCCUGCUGCUCACAUUGAUGGGUG | |
| | | | GCUAUUAAAGGCUAGCGAAUUCGAUAUCCAGCGGCCGCAAAAAAAAAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAAAAAAAAAAAAAAAAAAA | |
| IFNa | Pro009 | RNA266 | AGGGUUUCGUCUUUAGAUCUUCUGGUCCCCACAGACUCAGAGAGAACCCGCCACCAUGGCU | 465 |
| | | | CGGCUGUGCGCCUUCCUGAUGAUCCUGGUGAUGAUGAGCUACUACUGGAGCGCAUGCAGCC | |
| | | | UGGGCUGCGACCUGCCACACACCUACAACCUGGGCAAUAAGCGGGCUCUGACCGUGCUGGA | |
| | | | GGAGAUGCGGCGGCUGCCCCCCCUGAGCUGCCUGAAGGACCGGAAGGACUUCGGCUUCCCC | |
| | | | CUGGAGAAGGUGGACAACCAGCAGAUCCAAAAGGCACAGGCCAUCCUGGUGCUGCGGGACC | |
| | | | UGACCCAGCAGAUCCUGAACCUGUUCACCAGCAAGGACCUGAGCGCCACCUGGAACGCCAC | |
| | | | CCUGCUGGACAGCUUCUGCAACGACCUGCACCAGCAGCUGAACGACCUGAAGGCCUGCGUC | |
| | | | AUGCAGGAGCCACCACUGACCCAGGAGGACAGCCUGUUAGCUGUGCGGACUUACUUCCACC | |
| | | | GGAUCACCGUGUACCUGCGGAAGAAGAAGCACAGCCUGUGCGCUUGGGAGGUGAUCCGGGC | |
| | | | CGAGGUGUGGCGGGCCCUGAGCAGCAGCACCAACCUGCUGGCCCGGCUGAGCGAGGAGAAG | |
| | | | GAGUGACUAACUAAUUAAGCUGCCUUCUGCGGGGCUUGCCUUCUGGCCAUGCCCUUCUUCU | |
| | | | CUCCCUUGCACCUGUACCUCUUGGUCUUUGAAUAAAGCCUGAGUAGGAAGAAGCCUGCAUG | |
| | | | CCUGGUUCUCUGCGUCUGCGAAUUCGAUAUCCAGCGGCCGCGCUAGCGCUGAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAAAAAAAAAAAAAAAAAAAAAAAAAAAAAAAA | |
| IFNa | Pro009 | RNA853 | AGGGUUUCGUCUUUAGAUCUUCUGGUCCCCACAGACUCAGAGAGAACCCGCCACCAUGGCU | 466 |
| | | | CGGCUGUGCGCCUUCCUGAUGAUCCUGGUGAUGAUGAGCUACUACUGGAGCGCAUGCAGCC | |
| | | | UGGGCUGCGACCUGCCACACACCUACAACCUGGGCAAUAAGCGGGCUCUGACCGUGCUGGA | |
| | | | GGAGAUGCGGCGGCUGCCCCCCCUGAGCUGCCUGAAGGACCGGAAGGACUUCGGCUUCCCC | |
| | | | CUGGAGAAGGUGGACAACCAGCAGAUCCAAAAGGCACAGGCCAUCCUGGUGCUGCGGGACC | |
| | | | UGACCCAGCAGAUCCUGAACCUGUUCACCAGCAAGGACCUGAGCGCCACCUGGAACGCCAC | |
| | | | CCUGCUGGACAGCUUCUGCAACGACCUGCACCAGCAGCUGAACGACCUGAAGGCCUGCGUC | |
| | | | AUGCAGGAGCCACCACUGACCCAGGAGGACAGCCUGUUAGCUGUGCGGACUUACUUCCACC | |
| | | | GGAUCACCGUGUACCUGCGGAAGAAGAAGCACAGCCUGUGCGCUUGGGAGGUGAUCCGGGC | |
| | | | CGAGGUGUGGCGGGCCCUGAGCAGCAGCACCAACCUGCUGGCCCGGCUGAGCGAGGAGAAG | |
| | | | GAGUGACUAACUAAACCGGUGGACACAUGGUUACCACCUGGCCCUGAGUGCAGUCAGACUG | |
| | | | GGACAAAGGAGCCGUGAAACACGCAAGGAGCUUCUGGCUUCUCAGCUUUGAGACCUGGGCU | |
| | | | CUUUGGAGCACAGAGAACUUUGAUUUUUAAGCUUCUACAGAUAUGGUCCUAGAGACUGGGG | |
| | | | UCCUCAGACACUCCCGCUGUAUGUGGUGAGCUUGGCUGGAAACUUGCCAUAGCCAGGACUG | |
| | | | AGGGGCUGGCCCCAGGACGCUCUCAAGAAAUGGGGCUACUGUGGUUUAGACACUCCUGCUG | |
| | | | CUCACAUUGAUGGGUGGCUAUUAAAGGCUAGCGAAUUCGAUAUCCAGCGGCCGCAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAAAAAAAAAAAAAAAAAAAAAAAAAAAAAAAAAAA | |
| IL-2mutein | Pro010 | | AGGGUUUCGUCUUUAGAUCUUCUGGUCCCCACAGACUCAGAGAGAACCCGCCACCAUGUAC | 467 |
| | | | CGGAUGCAGCUGCUGAGCUGCAUCGCCCUGAGCCUGGCCCUGGUGACCAACAGCGCCCCCA | |
| | | | CCAGCAGCAGCACCAAGAAGACCCAGCUGCAACUGGAGCACCUGCUGCUGGAUCUGCAGAU | |
| | | | GAUCCUGAACGGCAUCAACAAUUACAAGAAUCCCAAGCUGACCCGGAUGCUGACCUUCAAG | |
| | | | UUCUACAUGCCCAAGAAGGCUACCGAGCUGAAGCACCUGCAGUGCCUGGAGGAGGAGCUGA | |
| | | | AGCCCCUGGAGGAGGUGCUGAACCUGGCCCAGAGCAAGAACUUCCACUUCGACCCCCGGGA | |
| | | | CGUGGUGAGCAACAUCAACGUGUUCGUGCUGGAGCUGAAGGGCAGCGAGACCACCUUCAUG | |
| | | | UGCGAGUACGCCGACGAGACCGCCACCAUCGUGGAGUUCCUGAACCGGUGGAUCACCUUCU | |
| | | | GCCAGAGCAUCAUCAGCAAAAAAAAAAAAAAAAAAAAAAAAAAAAAAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAA | |
| IL-2mutein, | Pro010 | RNA351 | AGGGUUUCGUCUUUAGAUCUUCUGGUCCCCACAGACUCAGAGAGAACCCGCCACCAUGUAC | 468 |
| RNA351-001 | | | CGGAUGCAGCUGCUGAGCUGCAUCGCCCUGAGCCUGGCCCUGGUGACCAACAGCGCCCCCA | |
| | | | CCAGCAGCAGCACCAAGAAGACCCAGCUGCAACUGGAGCACCUGCUGCUGGAUCUGCAGAU | |
| | | | GAUCCUGAACGGCAUCAACAAUUACAAGAAUCCCAAGCUGACCCGGAUGCUGACCUUCAAG | |
| | | | UUCUACAUGCCCAAGAAGGCUACCGAGCUGAAGCACCUGCAGUGCCUGGAGGAGGAGCUGA | |
| | | | AGCCCCUGGAGGAGGUGCUGAACCUGGCCCAGAGCAAGAACUUCCACUUCGACCCCCGGGA | |
| | | | CGUGGUGAGCAACAUCAACGUGUUCGUGCUGGAGCUGAAGGGCAGCGAGACCACCUUCAUG | |
| | | | UGCGAGUACGCCGACGAGACCGCCACCAUCGUGGAGUUCCUGAACCGGUGGAUCACCUUCU | |
| | | | GCCAGAGCAUCAUCAGCUGACUAACUAAACCGGUGGACACAUGGUUACCACCUGGCCCUGA | |
| | | | GUGCAGUCAGACUGGGACAAAGGAGCCGUGAAACACGCAAGGAGCUUCUGGCUUCUCAGCU | |
| | | | UUGAGACCUGGGCUCUUUGGAGCACAGAGAACUUUGAUUUUUAAGCUUCUACAGAUAUGGU | |
| | | | CCUAGAGACUGGGGUCCUCAGACACUCCCGCUGUAUGUGGUGAGCUUGGCUGGAAACUUGC | |
| | | | CAUAGCCAGGACUGAGGGGCUGGCCCCAGGACGCUCUCAAGAAAUGGGGCUACUGUGGUUU | |
| | | | AGACACUCCUGCUGCUCACAUUGAUGGGUGGCUAUUAAAGGCUAGCGAAUUCGAUAUCCAG | |
| | | | CGGCCGCAAAAAAAAAAAAAAAAAAAAAAAAAAAAAAAAAAAAAAAAAAAAAAAAAAAAAA | |
| | | | AAAAAAAAAAAAAAAAAAAAAAAAAAAAAAAAAAAAAAAAAAAAAAAAAAAAAAAA | |
| IL-15Ra | Pro045 | | AGGGUUUCGUCUUUAGAUCUUCUGGUCCCCACAGACUCAGAGAGAACCCGCCACCAUGAGC | 469 |
| (Sushi)- | | | GUGCUGACCCAGGUGCUGGCUCUGCUGCUGCUGUGGCUGACCGGCGCCCGGUGCACCACAU | |
| IL-15 | | | GCCCCCCCCCCGUGAGCAUCGAGCACGCCGACAUCCGGGUGAAGAACUACAGCGUGAACAG | |
| | | | CCGGGAGCGGUACGUGUGCAACAGCGGCUUCAAGCGGAAGGCCGGGACCAGCACCCUGAUC | |
| | | | GAGUGCGUGAUCAACAAGAACACCAACGUGGCCCACUGGACCACCCCCAGCCUGAAGUGCA | |
| | | | UCCGGGACCCAAGCCUGGCUGGCGGCUCAGGCGGUAGCGGGGGUAGCGGCGGCAGCGGCGG | |
| | | | CUCCGGAGGCAGCGGCGGAAAUUGGAUCGACGUGCGGUACGACCUGGAGAAGAUCGAGAGC | |
| | | | CUGAUCCAGAGCAUCCACAUCGACACCACCCUGUACACCGACAGCGACUUCCACCCUAGCU | |
| | | | GCAAGGUGACCGCCAUGAACUGCUUCCUGCUGGAGCUGCAGGUGAUCCUGCACGAGUACAG | |
| | | | CAACAUGACCCUGAACGAGACAGUGCGGAACGUGCUGUACCUGGCCAACAGCACCCUGAGC | |
| | | | AGCAACAAGAACGUGGCCGAGAGCGGCUGCAAGGAGUGCGAGGAGCUGGAGGAGAAGACCU | |
| | | | UCACCGAGUUCCUGCAGUCCUUCAUCCGGAUCGUGCAGAUGUUCAUCAACACCAGCUGACU | |
| | | | AACUAAUUAAGCUGCCUUCUGCGGGGCUUGCCUUCUGGCCAUGCCCUUCUUCUCUCCCUUG | |
| | | | CACCUGUACCUCUUGGUCUUUGAAUAAAGCCUGAGUAGGAAGAAGCCUGCAUGCCUGGUUC | |
| | | | UCUGCGUCUGCGAAUUCGAUAUCCAGCGGCCGCGCUAGCGCUGAAAAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAAAAAAAAAAAAAAAAAAAAAAAA | |
| IL-15Ra | Pro045 | RNA1114 | AGGGUUUCGUCUUUAGAUCUUCUGGUCCCCACAGACUCAGAGAGAACCCGCCACCAUGAGC | 470 |
| (Sushi)- | | | GUGCUGACCCAGGUGCUGGCUCUGCUGCUGCUGUGGCUGACCGGCGCCCGGUGCACCACAU | |
| IL-15 | | | GCCCCCCCCCCGUGAGCAUCGAGCACGCCGACAUCCGGGUGAAGAACUACAGCGUGAACAG | |
| | | | CCGGGAGCGGUACGUGUGCAACAGCGGCUUCAAGCGGAAGGCCGGGACCAGCACCCUGAUC | |
| | | | GAGUGCGUGAUCAACAAGAACACCAACGUGGCCCACUGGACCACCCCCAGCCUGAAGUGCA | |
| | | | UCCGGGACCCAAGCCUGGCUGGCGGCUCAGGCGGUAGCGGGGGUAGCGGCGGCAGCGGCGG | |
| | | | CUCCGGAGGCAGCGGCGGAAAUUGGAUCGACGUGCGGUACGACCUGGAGAAGAUCGAGAGC | |
| | | | CUGAUCCAGAGCAUCCACAUCGACACCACCCUGUACACCGACAGCGACUUCCACCCUAGCU | |
| | | | GCAAGGUGACCGCCAUGAACUGCUUCCUGCUGGAGCUGCAGGUGAUCCUGCACGAGUACAG | |
| | | | CAACAUGACCCUGAACGAGACAGUGCGGAACGUGCUGUACCUGGCCAACAGCACCCUGAGC | |
| | | | AGCAACAAGAACGUGGCCGAGAGCGGCUGCAAGGAGUGCGAGGAGCUGGAGGAGAAGACCU | |
| | | | UCACCGAGUUCCUGCAGUCCUUCAUCCGGAUCGUGCAGAUGUUCAUCAACACCAGCUGACU | |
| | | | AACUAAACCGGUGGACACAUGGUUACCACCUGGCCCUGAGUGCAGUCAGACUGGGACAAAG | |
| | | | GAGCCGUGAAACACGCAAGGAGCUUCUGGCUUCUCAGCUUUGAGACCUGGGCUCUUUGGAG | |
| | | | CACAGAGAACUUUGAUUUUUAAGCUUCUACAGAUAUGGUCCUAGAGACUGGGGUCCUCAGA | |
| | | | CACUCCCGCUGUAUGUGGUGAGCUUGGCUGGAAACUUGCCAUAGCCAGGACUGAGGGGCUG | |
| | | | GCCCCAGGACGCUCUCAAGAAAUGGGGCUACUGUGGUUUAGACACUCCUGCUGCUCACAUU | |
| | | | GAUGGGUGGCUAUUAAAGGCUAGCGAAUUCGAUAUCCAGCGGCCGCAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAAAAAAAAAAAAAAAAAAAAAAAAAAA | |
| mRNA | Pro040 | | AGGGUUUCGUCUUUAGAUCUUCUGGUCCCCACAGACUCAGAGAGAACCCGCCACCGACGAC | 470 |
| control: | | | GACAUCGCCGCCCUGGUGGUGGAUAACGGCAGCGGCAUGUGCAAGGCCGGCUUCGCCGGCG | |
| RNA1046- | | | ACGAUGCCCCCCGGGCCGUGUUCCCCAGCAUCGUCGGACGUCCCCGGCACCAGGGCGUGAU | |
| 001 | | | GGUGGGCAUGGGCCAGAAGGACUCCUACGUGGGCGACGAGGCCCAGAGCAAGCGGGGCAUC | |
| | | | CUGACCCUGAAGUACCCAAUCGAGCACGGCAUCGUGACCAACUGGGACGACAUGGAGAAGA | |
| | | | UCUGGCACCACACCUUCUACAAUGAGCUGCGGGUGGCCCCAGAGGAGCACCCCGUGCUGCU | |
| | | | GACCGAGGCCCCCCUGAACCCCAAGGCCAACCGGGAGAAGAUGACUCAGAUCAUGUUCGAG | |
| | | | ACCUUUAACACCCCCGCCAUGUACGUGGCCAUUCAGGCCGUGCUGAGCCUGUACGCCUCAG | |
| | | | GCCGGACCACCGGCAUCGUGAUGGACAGCGGCGACGGCGUGACCCACACCGUGCCCAUCUA | |
| | | | CGAGGGCUACGCCCUGCCCCACGCCAUCCUGCGGCUGGACCUGGCCGGCCGGGACCUGACC | |
| | | | GACUACCUGAUGAAGAUCCUGACCGAGCGGGGCUACAGCUUCACCACCACCGCCGAGCGGG | |
| | | | AGAUCGUGCGGGACAUCAAGGAGAAGCUGUGCUACGUGGCCCUGGACUUCGAGCAGGAGAU | |
| | | | GGCCACCGCCGCCAGCAGCAGCAGCCUGGAGAAGAGCUACGAGCUGCCCGACGGACAGGUG | |
| | | | AUCACCAUCGGCAACGAGCGGUUCCGUUGCCCCGAGGCCCUGUUCCAGCCCUCAUUCCUGG | |
| | | | GCAUGGAGAGCUGCGGCAUCCACGAGACCACCUUCAACAGCAUCAUGAAGUGCGACGUGGA | |
| | | | CAUCCGGAAGGAUCUGUACGCCAACACCGUGCUGAGCGGCGGCACCACCAUGUACCCCGGC | |
| | | | AUUGCCGAUCGGAUGCAGAAGGAGAUCACCGCGCUGGCCCCAAGCACCAUGAAGAUCAAGA | |
| | | | UCAUCGCUCCCCCCGAGCGGAAGUACAGCGUGUGGAUCGGCGGCAGCAUACUGGCUAGCCU | |
| | | | GAGCACCUUCCAGCAGAUGUGGAUCAGCAAGCAGGAGUACGACGAGAGCGGCCCUAGCAUC | |
| | | | GUGCACCGGAAAUGCUUCUGACUAACUAAUUAAGCUGCCUUCUGCGGGGCUUGCCUUCUGG | |
| | | | CCAUGCCCUUCUUCUCUCCCUUGCACCUGUACCUCUUGGUCUUUGAAUAAAGCCUGAGUAG | |
| | | | GAAGAAGCCUGCAUGCCUGGUUCUCUGCGUCUGCGAAUUCGAUAUCCAGCGGCCGCGCUAG | |
| | | | CGCUGAAAAAAAAAAAAAAAAAAAAAAAAAAAAAAAAAAAAAAAAAAAAAAAAAAAAAAAA | |
| | | | AAAAAAAAAAAAAAAAAAAAAAAAAAAAAAAAAAAAAAAAAAAAAAAAAAAAAA | |
| mRNA | Pro040 | RNA1046 | AGGGUUUCGUCUUUAGAUCUUCUGGUCCCCACAGACUCAGAGAGAACCCGCCACCGACGAC | 472 |
| control: | | | GACAUCGCCGCCCUGGUGGUGGAUAACGGCAGCGGCAUGUGCAAGGCCGGCUUCGCCGGCG | |
| RNA1046- | | | ACGAUGCCCCCCGGGCCGUGUUCCCCAGCAUCGUCGGACGUCCCCGGCACCAGGGCGUGAU | |
| 001 | | | GGUGGGCAUGGGCCAGAAGGACUCCUACGUGGGCGACGAGGCCCAGAGCAAGCGGGGCAUC | |
| | | | CUGACCCUGAAGUACCCAAUCGAGCACGGCAUCGUGACCAACUGGGACGACAUGGAGAAGA | |
| | | | UCUGGCACCACACCUUCUACAAUGAGCUGCGGGUGGCCCCAGAGGAGCACCCCGUGCUGCU | |
| | | | GACCGAGGCCCCCCUGAACCCCAAGGCCAACCGGGAGAAGAUGACUCAGAUCAUGUUCGAG | |
| | | | ACCUUUAACACCCCCGCCAUGUACGUGGCCAUUCAGGCCGUGCUGAGCCUGUACGCCUCAG | |
| | | | GCCGGACCACCGGCAUCGUGAUGGACAGCGGCGACGGCGUGACCCACACCGUGCCCAUCUA | |
| | | | CGAGGGCUACGCCCUGCCCCACGCCAUCCUGCGGCUGGACCUGGCCGGCCGGGACCUGACC | |
| | | | GACUACCUGAUGAAGAUCCUGACCGAGCGGGGCUACAGCUUCACCACCACCGCCGAGCGGG | |
| | | | AGAUCGUGCGGGACAUCAAGGAGAAGCUGUGCUACGUGGCCCUGGACUUCGAGCAGGAGAU | |
| | | | GGCCACCGCCGCCAGCAGCAGCAGCCUGGAGAAGAGCUACGAGCUGCCCGACGGACAGGUG | |
| | | | AUCACCAUCGGCAACGAGCGGUUCCGUUGCCCCGAGGCCCUGUUCCAGCCCUCAUUCCUGG | |
| | | | GCAUGGAGAGCUGCGGCAUCCACGAGACCACCUUCAACAGCAUCAUGAAGUGCGACGUGGA | |
| | | | CAUCCGGAAGGAUCUGUACGCCAACACCGUGCUGAGCGGCGGCACCACCAUGUACCCCGGC | |
| | | | AUUGCCGAUCGGAUGCAGAAGGAGAUCACCGCGCUGGCCCCAAGCACCAUGAAGAUCAAGA | |
| | | | UCAUCGCUCCCCCCGAGCGGAAGUACAGCGUGUGGAUCGGCGGCAGCAUACUGGCUAGCCU | |
| | | | GAGCACCUUCCAGCAGAUGUGGAUCAGCAAGCAGGAGUACGACGAGAGCGGCCCUAGCAUC | |
| | | | GUGCACCGGAAAUGCUUCUGACUAACUAAACCGGUGGACACAUGGUUACCACCUGGCCCUG | |
| | | | AGUGCAGUCAGACUGGGACAAAGGAGCCGUGAAACACGCAAGGAGCUUCUGGCUUCUCAGC | |
| | | | UUUGAGACCUGGGCUCUUUGGAGCACAGAGAACUUUGAUUUUUAAGCUUCUACAGAUAUGG | |
| | | | UCCUAGAGACUGGGGUCCUCAGACACUCCCGCUGUAUGUGGUGAGCUUGGCUGGAAACUUG | |
| | | | CCAUAGCCAGGACUGAGGGGCUGGCCCCAGGACGCUCUCAAGAAAUGGGGCUACUGUGGUU | |
| | | | UAGACACUCCUGCUGCUCACAUUGAUGGGUGGCUAUUAAAGGCUAGCGAAUUCGAUAUCCA | |
| | | | GCGGCCGCAAAAAAAAAAAAAAAAAAAAAAAAAAAAAAAAAAAAAAAAAAAAAAAAAAAAA | |
| | | | AAAAAAAAAAAAAAAAAAAAAAAAAAAAAAAAAAAAAAAAAAAAAAAAAAAAAAAAA | |
| mKR-336: | Pro034 | | AGGGUUUCGUCUUUAGAUCUUCUGGUCCCCACAGACUCAGAGAGAACCCGCCACCAUGGAG | 473 |
| RNA1047-001 | | | ACCGACACCCUGCUGCUGUGGGUGCUGCUGCUAUGGGUGCCCGGCAGCACCGGCAUGUGGG | |
| | | | AGCUGGAGAAGGACGUGUACGUGGUGGAGGUGGACUGGACCCCAGACGCUCCCGGCGAGAC | |
| | | | CGUGAACCUGACCUGCGACACCCCCGAGGAGGACGACAUCACUUGGACCAGCGACCAGCGG | |
| | | | CACGGCGUGAUCGGCAGCGGCAAGACCCUGACUAUCACCGUGAAGGAGUUCCUGGACGCCG | |
| | | | GCCAGUACACCUGCCACAAGGGCGGCGAGACCCUGAGCCACAGCCACCUGCUGCUACACAA | |
| | | | GAAGGAGAACGGCAUCUGGAGCACCGAGAUCCUGAAGAACUUCAAGAACAAGACCUUCCUG | |
| | | | AAGUGCGAGGCCCCCAACUACAGCGGCCGGUUCACAUGCAGCUGGCUGGUGCAGAGGAACA | |
| | | | UGGACCUGAAGUUCAACAUCAAGUCAAGCAGCAGCAGCCCCGACAGCCGGGCCGUGACCUG | |
| | | | CGGCAUGGCCAGCCUGAGCGCCGAGAAGGUGACCCUGGACCAGCGGGACUACGAGAAGUAC | |
| | | | AGCGUGAGCUGCCAGGAGGACGUCACCUGCCCCACAGCCGAGGAGACCCUGCCCAUCGAGC | |
| | | | UGGCCCUGGAGGCCCGGCAGCAGAACAAGUACGAGAACUACAGCACCAGCUUCUUCAUCCG | |
| | | | GGACAUCAUCAAGCCCGACCCACCCAAGAACCUGCAGAUGAAGCCCCUGAAGAACAGCCAG | |
| | | | GUGGAGGUGAGCUGGGAGUACCCAGACAGCUGGAGCACCCCCCACAGCUACUUCAGCCUGA | |
| | | | AGUUCUUCGUGCGGAUCCAGCGGAAGAAGGAGAAGAUGAAGGAGACCGAGGAGGGCUGCAA | |
| | | | CCAGAAGGGCGCCUUCCUGGUGGAGAAGACUAGCACCGAGGUGCAGUGCAAGGGCGGCAAC | |
| | | | GUGUGCGUGCAGGCACAGGAUCGGUACUACAACAGCAGCUGCAGCAAGUGGGCCUGCGUGC | |
| | | | CCUGCCGGGUGCGGAGCGGCGGCGGGGGCAGCGGAGGGGGGGGCAGCGGCGGGGGCGGUAG | |
| | | | CCGGGUGAUACCCGUGAGCGGCCCAGCACGGUGCCUGAGCCAGAGCAGGAACCUGCUGAAG | |
| | | | ACCACCGACGACAUGGUGAAGACCGCCCGGGAGAAGCUGAAGCACUACAGCUGCACCGCCG | |
| | | | AGGACAUUGACCACGAGGACAUCACCCGGGACCAGACCAGCACCCUGAAGACCUGCCUGCC | |
| | | | CCUGGAGCUGCACAAGAACGAGAGCUGUCUGGCCACCCGGGAGACCAGCAGCACCACCAGG | |
| | | | GGCAGCUGCUUACCACCCCAGAAGACCAGCCUGAUGAUGACCCUGUGCCUGGGCAGCAUCU | |
| | | | ACGAGGACCUGAAGAUGUACCAGACCGAGUUCCAGGCCAUCAACGCAGCCCUGCAGAACCA | |
| | | | CAACCACCAGCAGAUCAUCCUGGACAAGGGCAUGCUGGUGGCUAUCGACGAGCUGAUGCAG | |
| | | | AGCCUGAACCACAACGGCGAGACACUGCGGCAGAAGCCCCCCGUGGGCGAGGCCGACCCCU | |
| | | | ACCGGGUGAAGAUGAAGCUGUGCAUCCUGCUACACGCCUUCAGCACCCGGGUGGUGACCAU | |
| | | | CAACCGGGUGAUGGGCUACCUGAGCAGCGCCCACCCCGUGGGCCUGCUCGCCCGGGUGCCU | |
| | | | CUGAGCCUGUACAGCGGCCACCCCGUGGGCCUGCUGGCCCGGGUGCCCCUGAGCCUAUACA | |
| | | | GCGGCCUGAGCGGCCGGAGCGACAACCACGGCGGCGGAUCAGGCGGCGGAAGCCAGCUGGG | |
| | | | CGCUAGCGGCCCCGGCGAUGGCUGCUGUGUGGAGAAGACCAGCUUCCCCGAGGGCGCCAGC | |
| | | | GGCAGCCCCCUGGGCCCUAGGAACCUGAGCUGCUACCGGGUGAGCAAGACCGACUACGAGU | |
| | | | GCAGCUGGCAGUACGACGGCCCCGAGGACAACGUGAGCCACGUGCUGUGGUGCUGCUUCGU | |
| | | | GCCCCCCAACCACACCCACACCGGCCAGGAGCGGUGCCGGUACUUCAGCAGCGGCCCCGAC | |
| | | | CGGACCGUGCAGUUCUGGGAGCAGGACGGCAUCCCCGUGCUGAGCAAGGUGAACUUCUGGG | |
| | | | UGGAGAGCAGGCUGGGCAACCGGACCAUGAAGAGCCAGAAGAUCAGCCAGUACCUGUACAA | |
| | | | CUGGACCAAGACAACCCCCCCCCUGGGCCACAUCAAGGUGAGCCAGAGCCACCGGCAGCUG | |
| | | | CGGAUGGACUGGAACGUGUCAGAGGAGGCCGGCGCAGAGGUGCAGUUCCGGCGGAGGAUGC | |
| | | | CCACCACCAAUUGGACCCUGGGCGACUGCGGCCCCCAGGUGAACAGCGGCAGCGGAGUGCU | |
| | | | GGGCGAUAUCAGGGGCAGCAUGAGCGAGAGCUGCCUGUGCCCCAGCGAGAACAUGGCUCAG | |
| | | | GAGAUCCAGAUCCGGCGGCGGAGGCGGCUGAGCAGCGGCGCCCCCGGCGGCCCCUGGAGCG | |
| | | | ACUGGAGCAUGCCUGUGUGCGUGCCACCAGAGGUGCUGCCUCAGGCCGGCGGcGGGGGAAG | |
| | | | CGGCGGUGGCGGCAGCGGCGGAGGAGGCAGCGGCCCCACCAUCAAGCCCUGCCCCCCUUGC | |
| | | | AAGUGCCCUGCCCCAAACGCAGCCGGCGGCCCCAGCGUGUUCAUCUUCCCCCCAAAGAUCA | |
| | | | AGGACGUGCUGAUGAUCAGCCUGAGCCCCAUCGUGACCUGCGUGGUGGUGGACGUGAGCGA | |
| | | | GGACGACCCCGACGUGCAGAUCAGCUGGUUCGUGAACAACGUGGAGGUGCACACCGCCCAG | |
| | | | ACCCAGACCCACCGGGAGGAUUACAACAGCACCCUUCGGGUGGUGAGCGCCCUGCCCAUCC | |
| | | | AGCACCAGGACUGGAUGAGCGGCAAGGAGUUCAAGUGCAAGGUGAACAACAAGGACCUGGG | |
| | | | CGCACCCAUAGAGCGGACCAUCUCAAAGCCCAAGGGCAGCGUGCGGGCCCCCCAGGUGUAC | |
| | | | GUGCUGCCUCCACCCGAGGAGGAGAUGACCAAGAAGCAGGUGACCCUGACCUGCAUGGUGA | |
| | | | CCGACUUCAUGCCCGAGGACAUCUACGUGGAGUGGACCAACAACGGCAAGACCGAGCUUAA | |
| | | | CUACAAGAACACCGAGCCCGUGCUGGACAGCGACGGCUCAUACUUCAUGUACAGCAAGCUG | |
| | | | CGGGUGGAGAAGAAGAACUGGGUGGAGCGGAACAGCUACAGCUGCAGCGUGGUGCACGAGG | |
| | | | GCCUGCACAACCACCACACUACCAAGAGCUUCAGCCGGACCCCCGGCUGACUAACUAAUUA | |
| | | | AGCUGCCUUCUGCGGGGCUUGCCUUCUGGCCAUGCCCUUCUUCUCUCCCUUGCACCUGUAC | |
| | | | CUCUUGGUCUUUGAAUAAAGCCUGAGUAGGAAGAAGCCUGCAUGCCUGGUUCUCUGCGUCU | |
| | | | GCGAAUUCGAUAUCCAGCGGCCGCGCUAGCGCUGAAAAAAAAAAAAAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAAAAAAAAAAAAAAA | |
| mKR-336: | Pro034 | RNA1047 | AGGGUUUCGUCUUUAGAUCUUCUGGUCCCCACAGACUCAGAGAGAACCCGCCACCAUGGAG | 474 |
| RNA1047-001 | | | ACCGACACCCUGCUGCUGUGGGUGCUGCUGCUAUGGGUGCCCGGCAGCACCGGCAUGUGGG | |
| | | | AGCUGGAGAAGGACGUGUACGUGGUGGAGGUGGACUGGACCCCAGACGCUCCCGGCGAGAC | |
| | | | CGUGAACCUGACCUGCGACACCCCCGAGGAGGACGACAUCACUUGGACCAGCGACCAGCGG | |
| | | | CACGGCGUGAUCGGCAGCGGCAAGACCCUGACUAUCACCGUGAAGGAGUUCCUGGACGCCG | |
| | | | GCCAGUACACCUGCCACAAGGGCGGCGAGACCCUGAGCCACAGCCACCUGCUGCUACACAA | |
| | | | GAAGGAGAACGGCAUCUGGAGCACCGAGAUCCUGAAGAACUUCAAGAACAAGACCUUCCUG | |
| | | | AAGUGCGAGGCCCCCAACUACAGCGGCCGGUUCACAUGCAGCUGGCUGGUGCAGAGGAACA | |
| | | | UGGACCUGAAGUUCAACAUCAAGUCAAGCAGCAGCAGCCCCGACAGCCGGGCCGUGACCUG | |
| | | | CGGCAUGGCCAGCCUGAGCGCCGAGAAGGUGACCCUGGACCAGCGGGACUACGAGAAGUAC | |
| | | | AGCGUGAGCUGCCAGGAGGACGUCACCUGCCCCACAGCCGAGGAGACCCUGCCCAUCGAGC | |
| | | | UGGCCCUGGAGGCCCGGCAGCAGAACAAGUACGAGAACUACAGCACCAGCUUCUUCAUCCG | |
| | | | GGACAUCAUCAAGCCCGACCCACCCAAGAACCUGCAGAUGAAGCCCCUGAAGAACAGCCAG | |
| | | | GUGGAGGUGAGCUGGGAGUACCCAGACAGCUGGAGCACCCCCCACAGCUACUUCAGCCUGA | |
| | | | AGUUCUUCGUGCGGAUCCAGCGGAAGAAGGAGAAGAUGAAGGAGACCGAGGAGGGCUGCAA | |
| | | | CCAGAAGGGCGCCUUCCUGGUGGAGAAGACUAGCACCGAGGUGCAGUGCAAGGGCGGCAAC | |
| | | | GUGUGCGUGCAGGCACAGGAUCGGUACUACAACAGCAGCUGCAGCAAGUGGGCCUGCGUGC | |
| | | | CCUGCCGGGUGCGGAGCGGCGGCGGGGGCAGCGGAGGGGGCGGCAGCGGCGGGGGCGGUAG | |
| | | | CCGGGUGAUACCCGUGAGCGGCCCAGCACGGUGCCUGAGCCAGAGCAGGAACCUGCUGAAG | |
| | | | ACCACCGACGACAUGGUGAAGACCGCCCGGGAGAAGCUGAAGCACUACAGCUGCACCGCCG | |
| | | | AGGACAUUGACCACGAGGACAUCACCCGGGACCAGACCAGCACCCUGAAGACCUGCCUGCC | |
| | | | CCUGGAGCUGCACAAGAACGAGAGCUGUCUGGCCACCCGGGAGACCAGCAGCACCACCAGG | |
| | | | GGCAGCUGCUUACCACCCCAGAAGACCAGCCUGAUGAUGACCCUGUGCCUGGGCAGCAUCU | |
| | | | ACGAGGACCUGAAGAUGUACCAGACCGAGUUCCAGGCCAUCAACGCAGCCCUGCAGAACCA | |
| | | | CAACCACCAGCAGAUCAUCCUGGACAAGGGCAUGCUGGUGGCUAUCGACGAGCUGAUGCAG | |
| | | | AGCCUGAACCACAACGGCGAGACACUGCGGCAGAAGCCCCCCGUGGGCGAGGCCGACCCCU | |
| | | | ACCGGGUGAAGAUGAAGCUGUGCAUCCUGCUACACGCCUUCAGCACCCGGGUGGUGACCAU | |
| | | | CAACCGGGUGAUGGGCUACCUGAGCAGCGCCCACCCCGUGGGCCUGCUCGCCCGGGUGCCU | |
| | | | CUGAGCCUGUACAGCGGCCACCCCGUGGGCCUGCUGGCCCGGGUGCCCCUGAGCCUAUACA | |
| | | | GCGGCCUGAGCGGCCGGAGCGACAACCACGGCGGCGGAUCAGGCGGCGGAAGCCAGCUGGG | |
| | | | CGCUAGCGGCCCCGGCGAUGGCUGCUGUGUGGAGAAGACCAGCUUCCCCGAGGGCGCCAGC | |
| | | | GGCAGCCCCCUGGGCCCUAGGAACCUGAGCUGCUACCGGGUGAGCAAGACCGACUACGAGU | |
| | | | GCAGCUGGCAGUACGACGGCCCCGAGGACAACGUGAGCCACGUGCUGUGGUGCUGCUUCGU | |
| | | | GCCCCCCAACCACACCCACACCGGCCAGGAGCGGUGCCGGUACUUCAGCAGCGGCCCCGAC | |
| | | | CGGACCGUGCAGUUCUGGGAGCAGGACGGCAUCCCCGUGCUGAGCAAGGUGAACUUCUGGG | |
| | | | UGGAGAGCAGGCUGGGCAACCGGACCAUGAAGAGCCAGAAGAUCAGCCAGUACCUGUACAA | |
| | | | CUGGACCAAGACAACCCCCCCCCUGGGCCACAUCAAGGUGAGCCAGAGCCACCGGCAGCUG | |
| | | | CGGAUGGACUGGAACGUGUCAGAGGAGGCCGGCGCAGAGGUGCAGUUCCGGCGGAGGAUGC | |
| | | | CCACCACCAAUUGGACCCUGGGCGACUGCGGCCCCCAGGUGAACAGCGGCAGCGGAGUGCU | |
| | | | GGGCGAUAUCAGGGGCAGCAUGAGCGAGAGCUGCCUGUGCCCCAGCGAGAACAUGGCUCAG | |
| | | | GAGAUCCAGAUCCGGCGGCGGAGGCGGCUGAGCAGCGGCGCCCCCGGCGGCCCCUGGAGCG | |
| | | | ACUGGAGCAUGCCUGUGUGCGUGCCACCAGAGGUGCUGCCUCAGGCCGGCGGCGGGGGAAG | |
| | | | CGGCGGUGGCGGCAGCGGCGGAGGAGGCAGCGGCCCCACCAUCAAGCCCUGCCCCCCUUGC | |
| | | | AAGUGCCCUGCCCCAAACGCAGCCGGCGGCCCCAGCGUGUUCAUCUUCCCCCCAAAGAUCA | |
| | | | AGGACGUGCUGAUGAUCAGCCUGAGCCCCAUCGUGACCUGCGUGGUGGUGGACGUGAGCGA | |
| | | | GGACGACCCCGACGUGCAGAUCAGCUGGUUCGUGAACAACGUGGAGGUGCACACCGCCCAG | |
| | | | ACCCAGACCCACCGGGAGGAUUACAACAGCACCCUUCGGGUGGUGAGCGCCCUGCCCAUCC | |
| | | | AGCACCAGGACUGGAUGAGCGGCAAGGAGUUCAAGUGCAAGGUGAACAACAAGGACCUGGG | |
| | | | CGCACCCAUAGAGCGGACCAUCUCAAAGCCCAAGGGCAGCGUGCGGGCCCCCCAGGUGUAC | |
| | | | GUGCUGCCUCCACCCGAGGAGGAGAUGACCAAGAAGCAGGUGACCCUGACCUGCAUGGUGA | |
| | | | CCGACUUCAUGCCCGAGGACAUCUACGUGGAGUGGACCAACAACGGCAAGACCGAGCUUAA | |
| | | | CUACAAGAACACCGAGCCCGUGCUGGACAGCGACGGCUCAUACUUCAUGUACAGCAAGCUG | |
| | | | CGGGUGGAGAAGAAGAACUGGGUGGAGCGGAACAGCUACAGCUGCAGCGUGGUGCACGAGG | |
| | | | GCCUGCACAACCACCACACUACCAAGAGCUUCAGCCGGACCCCCGGCUGACUAACUAAACC | |
| | | | GGUGGACACAUGGUUACCACCUGGCCCUGAGUGCAGUCAGACUGGGACAAAGGAGCCGUGA | |
| | | | AACACGCAAGGAGCUUCUGGCUUCUCAGCUUUGAGACCUGGGCUCUUUGGAGCACAGAGAA | |
| | | | CUUUGAUUUUUAAGCUUCUACAGAUAUGGUCCUAGAGACUGGGGUCCUCAGACACUCCCGC | |
| | | | UGUAUGUGGUGAGCUUGGCUGGAAACUUGCCAUAGCCAGGACUGAGGGGCUGGCCCCAGGA | |
| | | | CGCUCUCAAGAAAUGGGGCUACUGUGGUUUAGACACUCCUGCUGCUCACAUUGAUGGGUGG | |
| | | | CUAUUAAAGGCUAGCGAAUUCGAUAUCCAGCGGCCGCAAAAAAAAAAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAAAAAAAAAAAAAAAAAA | |
| mIL-12- | Pro037 | | AGGGUUUCGUCUUUAGAUCUUCUGGUCCCCACAGACUCAGAGAGAACCCGCCACCAUGGAG | 475 |
| control2a | | | ACCGACACCCUGCUGCUGUGGGUGCUGCUGCUUUGGGUGCCCGGCAGCACCGGCAUGUGGG | |
| | | | AGCUGGAGAAGGACGUGUACGUGGUGGAGGUGGACUGGACCCCCGACGCCCCCGGCGAGAC | |
| | | | CGUGAACCUGACCUGCGACACCCCCGAGGAGGACGAUAUCACAUGGACCAGCGACCAGCGG | |
| | | | CACGGCGUCAUCGGCAGCGGCAAGACUCUGACCAUCACCGUGAAGGAGUUCCUGGACGCCG | |
| | | | GCCAGUACACCUGCCACAAGGGCGGCGAGACCCUGAGCCACAGCCACCUGCUGCUCCACAA | |
| | | | GAAGGAGAACGGCAUCUGGAGCACCGAGAUCCUGAAGAACUUCAAGAAUAAGACCUUCCUG | |
| | | | AAGUGCGAGGCCCCCAACUACAGCGGCCGGUUCACCUGCAGCUGGCUGGUGCAGCGGAACA | |
| | | | UGGACCUGAAGUUCAACAUCAAGAGCAGCUCAAGCAGCCCCGACAGCCGGGCCGUGACCUG | |
| | | | CGGCAUGGCCAGCCUGAGCGCCGAGAAGGUGACCCUGGACCAGCGGGACUACGAGAAGUAC | |
| | | | UCAGUGAGCUGCCAGGAGGACGUGACCUGCCCCACAGCCGAGGAGACCCUGCCAAUCGAGC | |
| | | | UGGCCCUGGAGGCCCGGCAGCAGAACAAGUACGAGAACUACAGCACCAGCUUUUUCAUCCG | |
| | | | GGACAUUAUCAAGCCUGACCCCCCCAAGAACCUGCAGAUGAAGCCCCUGAAGAAUAGCCAG | |
| | | | GUGGAGGUGAGCUGGGAGUACCCCGACAGCUGGAGCACCCCCCACAGCUACUUCAGCCUGA | |
| | | | AGUUCUUCGUGCGGAUCCAGCGGAAGAAGGAGAAGAUGAAGGAGACCGAGGAGGGCUGUAA | |
| | | | CCAGAAGGGCGCCUUCCUGGUGGAGAAGACCAGCACCGAGGUGCAGUGCAAGGGCGGCAAC | |
| | | | GUGUGCGUGCAGGCCCAGGACCGGUACUACAACAGCAGCUGCAGCAAGUGGGCCUGCGUGC | |
| | | | CCUGCCGGGUGCGGAGCGGCGGCGGAGGAAGCGGCGGCGGCGGCAGCGGCGGAGGCGGCAG | |
| | | | CCGGGUGAUUCCCGUGAGCGGCCCCGCCCGGUGCCUGAGCCAGAGCCGGAACCUGCUGAAG | |
| | | | ACAACCGACGACAUGGUGAAGACCGCCCGGGAGAAGCUGAAGCACUACAGCUGCACCGCCG | |
| | | | AGGACAUCGACCACGAGGACAUCACCCGGGACCAGACCAGCACCCUGAAGACCUGCCUGCC | |
| | | | CCUGGAGCUGCACAAGAACGAGAGCUGCCUGGCCACCCGGGAGACCAGCUCAACCACCCGG | |
| | | | GGCAGCUGUCUGCCCCCACAGAAGACCUCCCUUAUGAUGACCCUGUGCCUGGGCAGCAUCU | |
| | | | ACGAGGACCUGAAGAUGUACCAGACCGAGUUCCAGGCCAUCAACGCCGCACUGCAGAACCA | |
| | | | CAACCACCAGCAGAUCAUCCUGGACAAGGGCAUGCUGGUGGCCAUCGACGAGCUGAUGCAG | |
| | | | AGCCUGAACCACAACGGAGAGACCCUGCGGCAGAAGCCCCCCGUGGGCGAGGCCGACCCAU | |
| | | | ACCGGGUGAAGAUGAAGCUGUGCAUCCUGCUGCACGCCUUCAGCACCCGGGUGGUGACCAU | |
| | | | CAACCGGGUGAUGGGCUACCUGAGCAGCGCCUGACUAACUAAUUAAGCUGCCUUCUGCGGG | |
| | | | GCUUGCCUUCUGGCCAUGCCCUUCUUCUCUCCCUUGCACCUGUACCUCUUGGUCUUUGAAU | |
| | | | AAAGCCUGAGUAGGAAGAAGCCUGCAUGCCUGGUUCUCUGCGUCUGCGAAUUCGAUAUCCA | |
| | | | GCGGCCGCGCUAGCGCUGAAAAAAAAAAAAAAAAAAAAAAAAAAAAAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAA | |
| mIL-12- | Pro037 | RNA981 | AGGGUUUCGUCUUUAGAUCUUCUGGUCCCCACAGACUCAGAGAGAACCCGCCACCAUGGAG | 476 |
| control2a | | | ACCGACACCCUGCUGCUGUGGGUGCUGCUGCUUUGGGUGCCCGGCAGCACCGGCAUGUGGG | |
| | | | AGCUGGAGAAGGACGUGUACGUGGUGGAGGUGGACUGGACCCCCGACGCCCCCGGCGAGAC | |
| | | | CGUGAACCUGACCUGCGACACCCCCGAGGAGGACGAUAUCACAUGGACCAGCGACCAGCGG | |
| | | | CACGGCGUCAUCGGCAGCGGCAAGACUCUGACCAUCACCGUGAAGGAGUUCCUGGACGCCG | |
| | | | GCCAGUACACCUGCCACAAGGGCGGCGAGACCCUGAGCCACAGCCACCUGCUGCUCCACAA | |
| | | | GAAGGAGAACGGCAUCUGGAGCACCGAGAUCCUGAAGAACUUCAAGAAUAAGACCUUCCUG | |
| | | | AAGUGCGAGGCCCCCAACUACAGCGGCCGGUUCACCUGCAGCUGGCUGGUGCAGCGGAACA | |
| | | | UGGACCUGAAGUUCAACAUCAAGAGCAGCUCAAGCAGCCCCGACAGCCGGGCCGUGACCUG | |
| | | | CGGCAUGGCCAGCCUGAGCGCCGAGAAGGUGACCCUGGACCAGCGGGACUACGAGAAGUAC | |
| | | | UCAGUGAGCUGCCAGGAGGACGUGACCUGCCCCACAGCCGAGGAGACCCUGCCAAUCGAGC | |
| | | | UGGCCCUGGAGGCCCGGCAGCAGAACAAGUACGAGAACUACAGCACCAGCUUUUUCAUCCG | |
| | | | GGACAUUAUCAAGCCUGACCCCCCCAAGAACCUGCAGAUGAAGCCCCUGAAGAAUAGCCAG | |
| | | | GUGGAGGUGAGCUGGGAGUACCCCGACAGCUGGAGCACCCCCCACAGCUACUUCAGCCUGA | |
| | | | AGUUCUUCGUGCGGAUCCAGCGGAAGAAGGAGAAGAUGAAGGAGACCGAGGAGGGCUGUAA | |
| | | | CCAGAAGGGCGCCUUCCUGGUGGAGAAGACCAGCACCGAGGUGCAGUGCAAGGGCGGCAAC | |
| | | | GUGUGCGUGCAGGCCCAGGACCGGUACUACAACAGCAGCUGCAGCAAGUGGGCCUGCGUGC | |
| | | | CCUGCCGGGUGCGGAGCGGCGGCGGAGGAAGCGGCGGCGGCGGCAGCGGCGGAGGCGGCAG | |
| | | | CCGGGUGAUUCCCGUGAGCGGCCCCGCCCGGUGCCUGAGCCAGAGCCGGAACCUGCUGAAG | |
| | | | ACAACCGACGACAUGGUGAAGACCGCCCGGGAGAAGCUGAAGCACUACAGCUGCACCGCCG | |
| | | | AGGACAUCGACCACGAGGACAUCACCCGGGACCAGACCAGCACCCUGAAGACCUGCCUGCC | |
| | | | CCUGGAGCUGCACAAGAACGAGAGCUGCCUGGCCACCCGGGAGACCAGCUCAACCACCCGG | |
| | | | GGCAGCUGUCUGCCCCCACAGAAGACCUCCCUUAUGAUGACCCUGUGCCUGGGCAGCAUCU | |
| | | | ACGAGGACCUGAAGAUGUACCAGACCGAGUUCCAGGCCAUCAACGCCGCACUGCAGAACCA | |
| | | | CAACCACCAGCAGAUCAUCCUGGACAAGGGCAUGCUGGUGGCCAUCGACGAGCUGAUGCAG | |
| | | | AGCCUGAACCACAACGGAGAGACCCUGCGGCAGAAGCCCCCCGUGGGCGAGGCCGACCCAU | |
| | | | ACCGGGUGAAGAUGAAGCUGUGCAUCCUGCUGCACGCCUUCAGCACCCGGGUGGUGACCAU | |
| | | | CAACCGGGUGAUGGGCUACCUGAGCAGCGCCUGACUAACUAAACCGGUGGACACAUGGUUA | |
| | | | CCACCUGGCCCUGAGUGCAGUCAGACUGGGACAAAGGAGCCGUGAAACACGCAAGGAGCUU | |
| | | | CUGGCUUCUCAGCUUUGAGACCUGGGCUCUUUGGAGCACAGAGAACUUUGAUUUUUAAGCU | |
| | | | UCUACAGAUAUGGUCCUAGAGACUGGGGUCCUCAGACACUCCCGCUGUAUGUGGUGAGCUU | |
| | | | GGCUGGAAACUUGCCAUAGCCAGGACUGAGGGGCUGGCCCCAGGACGCUCUCAAGAAAUGG | |
| | | | GGCUACUGUGGUUUAGACACUCCUGCUGCUCACAUUGAUGGGUGGCUAUUAAAGGCUAGCG | |
| | | | AAUUCGAUAUCCAGCGGCCGCAAAAAAAAAAAAAAAAAAAAAAAAAAAAAAAAAAAAAAAA | |
| | | | AAAAAAAAAAAAAAAAAAAAAAAAAAAAAAAAAAAAAAAAAAAAAAAAAAAAAAAAAAAAA | |
| | | | AAAAAAAAA | |

TABLE 13
Exemplary amino acid sequence of a therapeutic described herein

| | | SEQ | |
|---|---|---|---|
| | | ID | |
| Notes | Amino acid sequence | NO.. | Notes |
| Pro010 | MYRMQLLSCIALSLALVINSAPTSSSTKKTQLQLEHLLLDLQMILNGINN | SEQ | IL-2 |
| | YKNPKLTRMLTFKFYMPKKATELKHLQCLEEELKPLEEVLNLAQSKNFH | ID | |
| | FDPRDVVSNINVFVLELKGSETTFMCEYADETATIVEFLNRWITFCQSIIS | NO.. | |
| | | 501 | |
| Pro065 | MEFGLSWVFLVALFRGVQCAPTSSSTKKTQLQLEHLLLDLQMILNGINN | SEQ | IL-2 |
| | YKNPKLTRMLTFKFYMPKKATELKHLQCLEEELKPLEEVLNLAQSKNFH | ID | |
| | LRPRDLISNINVIVLELKGSETTFMCEYADETATIVEFLNRWITFCQSIISTL | NO.. | |
| | T | 502 | |
| Pro006 | MCPQKLTISWFAIVLLVSPLMAMWELEKDVYVVEVDWTPDAPGETVNL | SEQ | IL-12 |
| | TCDTPEEDDITWTSDQRHGVIGSGKTLTITVKEFLDAGQYTCHKGGETLS | ID | p35- |
| | HSHLLLHKKENGIWSTEILKNFKNKTFLKCEAPNYSGRFTCSWLVQRNM | NO.. | IgG |
| | DLKFNIKSSSSSPDSRAVTCGMASLSAEKVTLDQRDYEKYSVSCQEDVT | 503 | |
| | CPTAEETLPIELALEARQQNKYENYSTSFFIRDIIKPDPPKNLQMKPLKNS | | |
| | QVEVSWEYPDSWSTPHSYFSLKFFVRIQRKKEKMKETEEGCNQKGAFLV | | |
| | EKTSTEVQCKGGNVCVQAQDRYYNSSCSKWACVPCRVRSGGGGSGGG | | |
| | GSGGGGSRVIPVSGPARCLSQSRNLLKTTDDMVKTAREKLKHYSCTAED | | |
| | IDHEDITRDQTSTLKTCLPLELHKNESCLATRETSSTTRGSCLPPQKTSLM | | |
| | MTLCLGSIYEDLKMYQTEFQAINAALQNHNHQQIILDKGMLVAIDELMQ | | |
| | SLNHNGETLRQKPPVGEADPYRVKMKLCILLHAFSTRVVTINRVMGYLS | | |
| | SASGGGSGGGGSGGGGSGGGGSGGGSLQVVISAILALVVLTVISLIILIGG | | |
| | GGSGKPIPNPLLGLDST | | |
| Pro078 | MEFGLSWVFLVALFRGVQCRNLPVATPDPGMFPCLHHSQNLLRAVSNM | SEQ | IL-12 |
| | LQKARQTLEFYPCTSEEIDHEDITKDKTSTVEACLPLELTKNESCLNSRET | ID | p35- |
| | SFITNGSCLASRKTSFMMALCLSSIYEDLKMYQVEFKTMNAKLLMDPKR | NO.. | IgG |
| | QIFLDQNMLAVIDELMQALNFNSETVPQKSSLEEPDFYKTKIKLCILLHAF | 504 | |
| | RIRAVTIDRVMSYLNASHPVGLLARVPLSLYSGHPVGLLARVPLSLYSGL | | |
| | SGRSDNHGGGSGGGSPKSSDKTHTCPPCPAPEAAGGPSVFLFPPKPKDTL | | |
| | MISRTPEVTCVVVDVSHEDPEVKFNWYVDGVEVHNAKTKPREEQYNST | | |
| | YRVVSVLTVLHQDWLNGKEYKCKVSNKALGAPIEKTISKAKGQPREPQ | | |
| | VCTLPPSRDELTENQVSLTCLVKGFYPSDIAVEWESNGQPENNYKTTPPV | | |
| | LDSDGSFFLYSWLTVDKSRWQQGNVFSCSVMHEALHNHYTQKSLSLSP | | |
| | GK | | |
| Pro079 | MEFGLSWVFLVALFRGVQCIWELKKDVYVVELDWYPDAPGEMVVLTC | SEQ | IL-12 |
| | DTPEEDGITWTLDQSSEVLGSGKTLTIQVKEFGDAGQYTCHKGGEVLSH | ID | p35- |
| | SLLLLHKKEDGIWSTDILKDQKEPKNKTFLRCEAKNYSGRFTCWWLTTI | NO.. | IgG |
| | STDLTFSVKSSRGSSDPQGVTCGAATLSAERVRGDNKEYEYSVECQEDS | 505 | |
| | ACPAAEESLPIEVMVDAVHKLKYENYTSSFFIRDIIKPDPPKNLQLKPLKN | | |
| | SRQVEVSWEYPDTWSTPHSYFSLTFCVQVQGKSKREKKDRVFTDKTSAT | | |
| | VICRKNASISVRAQDRYYSSSWSEWASVPCSHPVGLLARVPLSLYSGHP | | |
| | VGLLARVPLSLYSGLSGRSDNHGGGSGGGSPKSSDKTHTCPPCPAPEAA | | |
| | GGPSVFLFPPKPKDTLMISRTPEVTCVVVDVSHEDPEVKFNWYVDGVEV | | |
| | HNAKTKPREEQYNSTYRVVSVLTVLHQDWLNGKEYKCKVSNKALGAPI | | |
| | EKTISKAKGQPREPRVYTLPPCRDELTKNQVSLTCLVKGFYPSDIAVEWE | | |
| | SNGQPENNYKTTPPVLVSDGSFTLYSKLTVDKSRWQQGNVFSCSVMHE | | |
| | ALHNHYTQKSLSLSPGK | | |
| Pro080 | MEFGLSWVFLVALFRGVQCCRTSECCFQDPPYPDADSGSASGPRDLRCY | SEQ | IL-12 |
| | RISSDRYECSWQYEGPTAGVSHFLRCCLSSGRCCYFAAGSATRLQFSDQ | ID | p35- |
| | AGVSVLYTVTLWVESWARNQTEKSPEVTLQLYNSVKYEPPLGDIKVSKL | NO.. | IgG |
| | AGQLRMEWETPDNQVGAEVQFRHRTPSSPWKLGDCGPQDDDTESCLCP | 506 | |
| | LEMNVAQEFQLRRRQLGSQGSSWSKWSSPVCVPPENPHPVGLLARVPLS | | |
| | LYSGHPVGLLARVPLSLYSGLSGRSDNHGGGSGGGSRNLPVATPDPGMF | | |
| | PCLHHSQNLLRAVSNMLQKARQTLEFYPCTSEEIDHEDITKDKTSTVEAC | | |
| | LPLELTKNESCLNSRETSFITNGSCLASRKTSFMMALCLSSIYEDLKMYQ | | |
| | VEFKTMNAKLLMDPKRQIFLDQNMLAVIDELMQALNENSETVPQKSSLE | | |
| | EPDFYKTKIKLCILLHAFRIRAVTIDRVMSYLNASHPVGLLARVPLSLYSG | | |
| | HPVGLLARVPLSLYSGLSGRSDNHGGGSGGGSPKSSDKTHTCPPCPAPEA | | |
| | AGGPSVFLFPPKPKDTLMISRTPEVTCVVVDVSHEDPEVKFNWYVDGVE | | |
| | VHNAKTKPREEQYNSTYRVVSVLTVLHQDWLNGKEYKCKVSNKALGA | | |
| | PIEKTISKAKGQPREPQVCTLPPSRDELTENQVSLTCLVKGFYPSDIAVEW | | |
| | ESNGQPENNYKTTPPVLDSDGSFFLYSWLTVDKSRWQQGNVFSCSVMH | | |
| | EALHNHYTQKSLSLSPGK | | |
| Pro081 | MEFGLSWVFLVALFRGVQCKIDACKRGDVTVKPSHVILLGSTVNITCSL | SEQ | IL-12 |
| | KPRQGCFHYSRRNKLILYKFDRRINFHHGHSLNSQVTGLPLGTTLFVCKL | ID | p35- |
| | ACINSDEIQICGAEIFVGVAPEQPQNLSCIQKGEQGTVACTWERGRDTHL | NO.. | IgG |
| | YTEYTLQLSGPKNLTWQKQCKDIYCDYLDFGINLTPESPESNFTAKVTA | 507 | |
| | VNSLGSSSSLPSTFTFLDIVRPLPPWDIRIKFQKASVSRCTLYWRDEGLVL | | |
| | LNRLRYRPSNSRLWNMVNVTKAKGRHDLLDLKPFTEYEFQISSKLHLYK | | |
| | GSWSDWSESLRAQTPEEHPVGLLARVPLSLYSGHPVGLLARVPLSLYSG | | |
| | LSGRSDNHGGGSGGGSIWELKKDVYVVELDWYPDAPGEMVVLTCDTPE | | |
| | EDGITWTLDQSSEVLGSGKTLTIQVKEFGDAGQYTCHKGGEVLSHSLLL | | |
| | LHKKEDGIWSTDILKDQKEPKNKTFLRCEAKNYSGRFTCWWLTTISTDL | | |
| | TFSVKSSRGSSDPQGVTCGAATLSAERVRGDNKEYEYSVECQEDSACPA | | |
| | AEESLPIEVMVDAVHKLKYENYTSSFFIRDIIKPDPPKNLQLKPLKNSRQV | | |
| | EVSWEYPDTWSTPHSYFSLTFCVQVQGKSKREKKDRVFTDKTSATVICR | | |
| | KNASISVRAQDRYYSSSWSEWASVPCSHPVGLLARVPLSLYSGHPVGLL | | |
| | ARVPLSLYSGLSGRSDNHGGGSGGGSPKSSDKTHTCPPCPAPEAAGGPS | | |
| | VFLFPPKPKDTLMISRTPEVTCVVVDVSHEDPEVKFNWYVDGVEVHNA | | |
| | KTKPREEQYNSTYRVVSVLTVLHQDWLNGKEYKCKVSNKALGAPIEKTI | | |
| | SKAKGQPREPRVYTLPPCRDELTKNQVSLTCLVKGFYPSDIAVEWESNG | | |
| | QPENNYKTTPPVLVSDGSFTLYSKLTVDKSRWQQGNVFSCSVMHEALH | | |
| | NHYTQKSLSLSPGK | | |
| Pro011 | MASPQLRGYGVQAIPVLLLLLLLLLLPLRVTPGTTCPPPVSIEHADIRVKN | SEQ | IL-15 |
| | YSVNSRERYVCNSGFKRKAGTSTLIECVINKNTNVAHWTTPSLKCIRDPS | ID | and |
| | LAHYSPVPTVVTPKVTSQPESPSPSAKEPEAFSPKSDTAMTTETAIMPGSR | NO.. | sushi |
| | LTPSQTTSAGTTGTGSHKSSRAPSLAATMTLEPTASTSLRITEISPHSSKMT | 508 | |
| | KVAISTSVLLVGAGVVMAFLAWYIKSRQPSQPCRVEVETMETVPMTVR | | |
| | ASSKEDEDTGAGGSGGSGGSGGSGGSGGSGGMKILKPYMRNTSISCYLC | | |
| | FLLNSHELTEAGIHVFILGCVSVGLPKTEANWIDVRYDLEKIESLIQSIHID | | |
| | TTLYTDSDFHPSCKVTAMNCFLLELQVILHEYSNMTLNETVRNVLYLAN | | |
| | STLSSNKNVAESGCKECEELEEKTFTEFLQSFIRIVQMFINTS | | |
| Pro096 | MEFGLSWVFLVALFRGVQCITCPPPMSVEHADIWVKSYSLYSRERYICNS | SEQ | IL-15 |
| | GFKRKAGTSSLTECVLNKATNVAHWTTPSLKCIRDPALVHQRPAPPGGS | ID | and |
| | GGSGGSGGSGGSGGSGGNWVNVISDLKKIEDLIQSMHIDATLYTESDVH | NO.. | sushi |
| | PSCKVTAMKCFLLELQVISLESGDASIHDTVENLIILANNSLSSNGNVTES | 509 | |
| | GCKECEELEEKNIKEFLQSFVHIVQMFINTS | | |
| Pro088 | MEFGLSWVFLVALFRGVQCCDLPQTHSLGSRRTLMLLAQMRRISLFSCL | SEQ | inter- |
| | KDRHDFGFPQEEFGNQFQKAETIPVLHEMIQQIFNLFSTKDSSAAWDETL | ID | feron |
| | LDKFYTELYQQLNDLEACVIQGVGVTETPLMKEDSILAVRKYFQRITLYL | NO.. | alpha |
| | KEKKYSPCAWEVVRAEIMRSFSLSTNLQESLRSKE | 510 | |

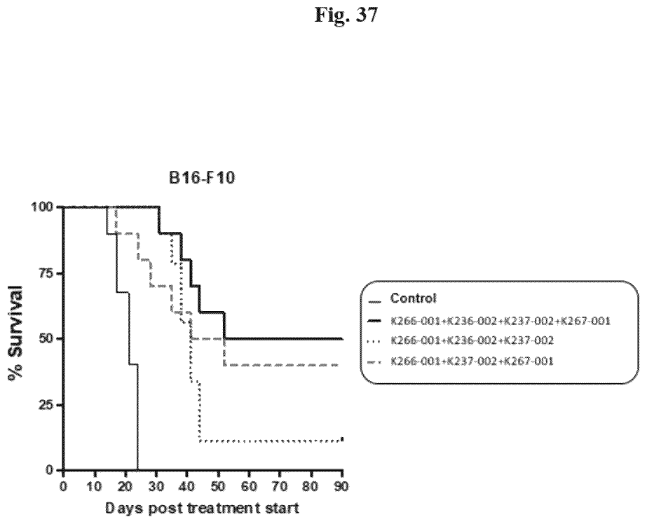

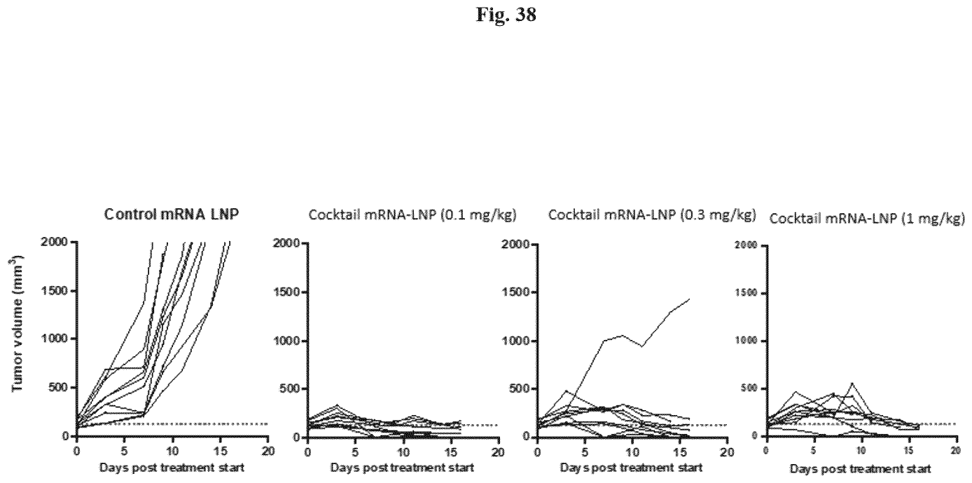

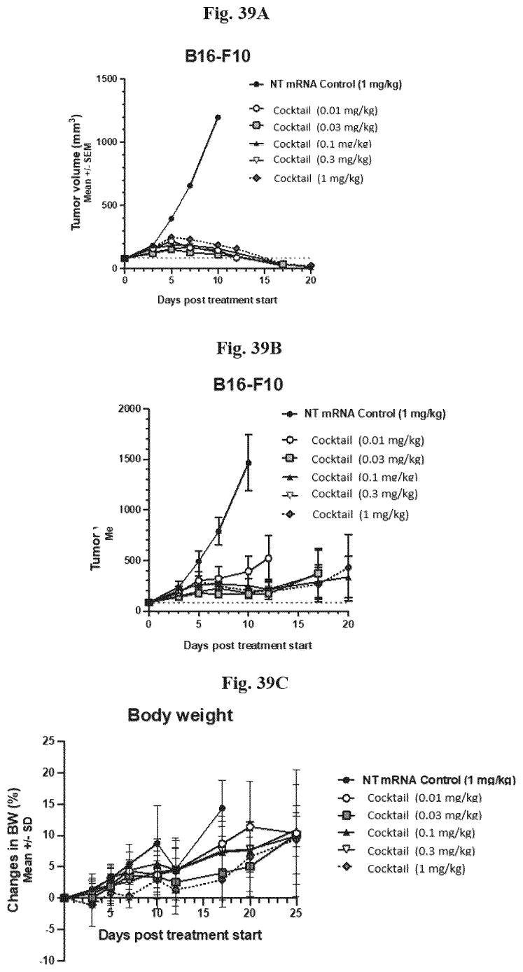

FIG. 36 illustrates treatment of subcutaneous B16-F10 melanoma tumors with cocktail mRNA LNPs (K266-001+K267-001+K237-002). Female C57BL/6NCrl mice were implanted with 2.5×10⁵ B16-F10 cells and randomized into treatment groups (n=10) on day 8 post implant based on tumor size. Mice received six intratumoral injections of either control mRNA or oncoselective mRNA LNP combination mRNA treatment formulated in LNPs (Q3Dx6). The treatment period is highlighted and the proportion of complete responders (CRs) is indicated. FIG. 37 illustrates overall survival after treatment with cocktail mRNA-LNPs and a control mRNA LNPs (cocktail mRNA LNPs vs. control mRNA p<0.0001; Cox regression). Cocktail mRNA combination treatments lacking an individual mRNA component are shown. FIG. 38 illustrates dose titration treatment of subcutaneous B16-F10 melanoma tumors with cocktail mRNA LNPs. Intratumoral injections started on day 0 in established tumors at an average size of 130 mm³. Treatments were administered on days 0, 2, 4, 7, 9, and 11 (n=10/group). Cocktail: mRNA-LNPs (Cocktail mRNA-LNP:K266-001+K236-002+K237-002+K267-001). Control: K253-006. FIGS. 39A-C illustrate, dose titration treatment of subcutaneous B16-F10 melanoma tumors with Cocktail mRNA-LNPs. Intratumoral injections started on day 0 in established tumors at an average size of 84 mm³. Treatments were administered on days 0, 2, 4, 7, 9, and 11 (n=10/group). FIG. 39A. Group Geomean. FIG. 39B. Mean values are shown for each group. FIG. 39C. Mean values+/−SD for body weight changes. Cocktail mRNA-LNPs: K250-005+K364-002+K366-002+K267-004; mtIL-12: K250-003, Pro006; IL-15: K236-003, Pro011; K237-003, Pro009; and IL-2 mutein: K267-002, Pro010.

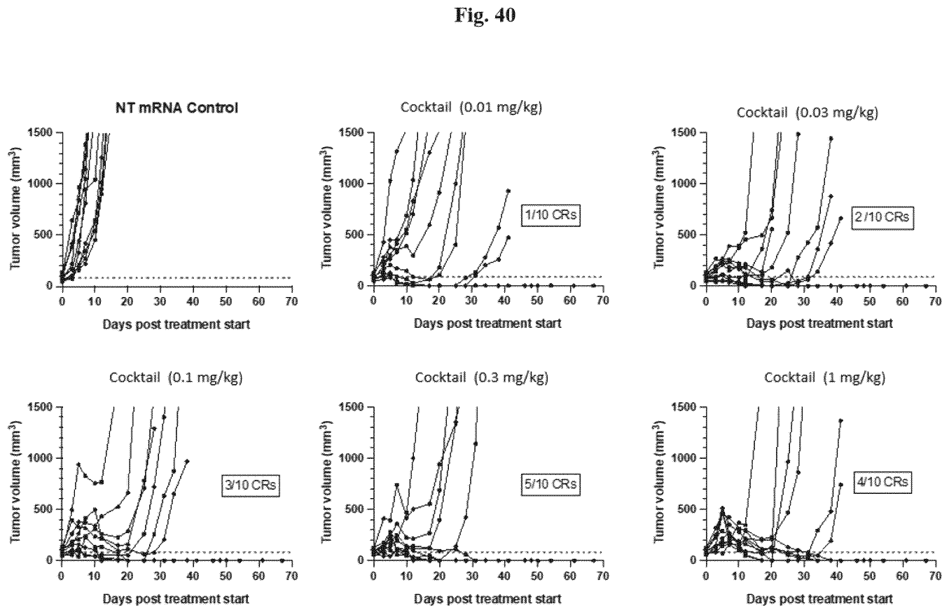

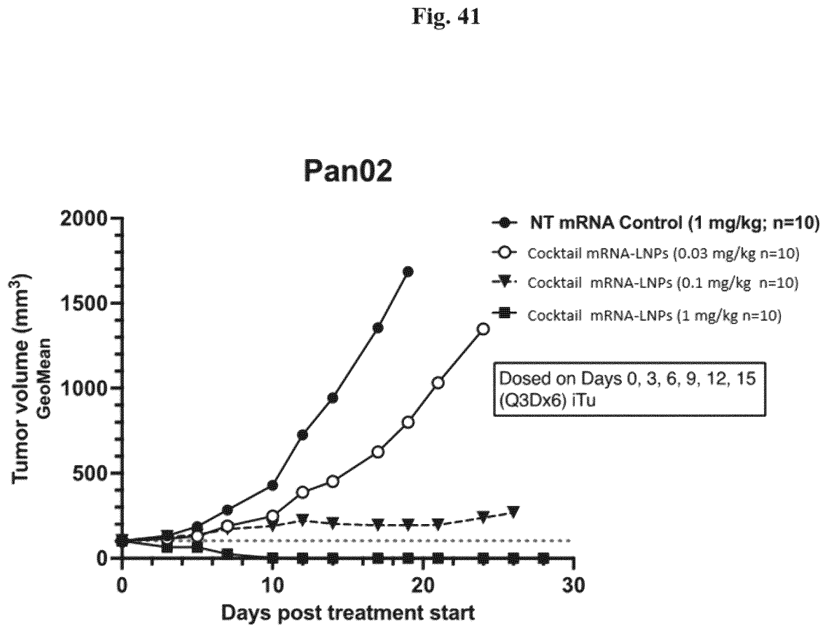

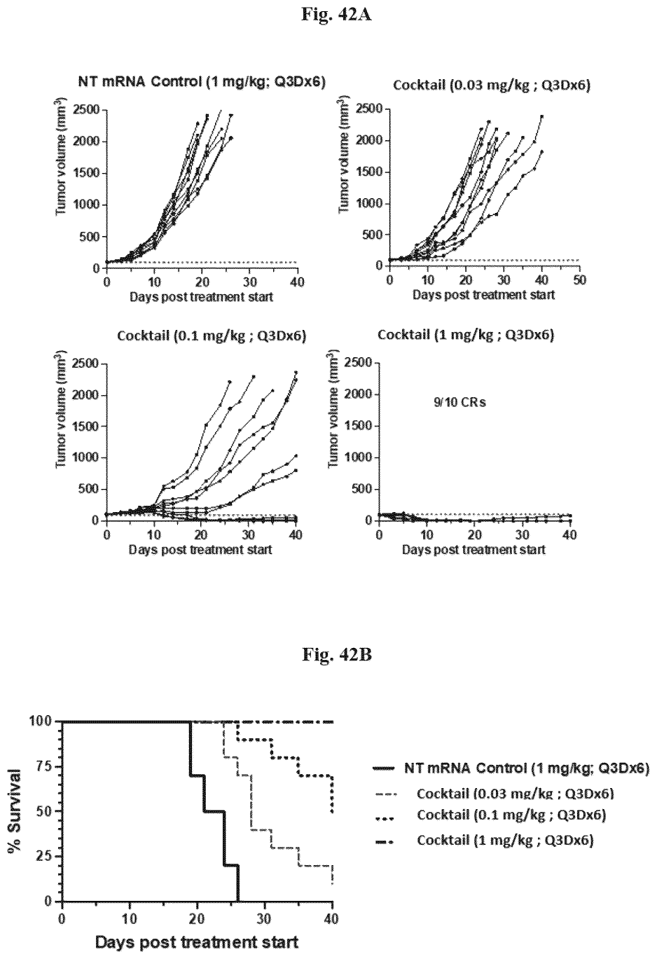

FIG. 40 illustrates individual tumor growth curves. Dose titration treatment of subcutaneous B16-F10 melanoma tumors Cocktail mRNA-LNPs. Intratumoral injections started on day 0 in established tumors at an average size of 84 mm³. Treatments were administered on days 0, 2, 4, 7, 9 and 11 (n=10/group). Cocktail mRNA-LNPs: K250-005+K364-002+K366-002+K267-004. FIG. 41 illustrates treatment of subcutaneous Pan02 pancreatic adenocarcinoma tumors with Cocktail mRNA-LNPs (Pro006, Pro045, Pro009, Pro010). 5×10⁶ cells were implanted subcutaneously on day-9. Intratumoral injections started on day 0 in established randomized tumors at an average size of 102 mm³. Treatments were administered on days 0, 3, 6, 9, 12 and 15 (n=10/group). Pro040 was the mRNA control. FIG. 42A illustrates Individual Pan02 tumor growth curves. Dose titration treatment of subcutaneous Pan02 syngeneic pancreatic tumors with Cocktail mRNA-LNPs (Pro006, Pro045, Pro009, Pro010). Intratumoral injections started on day 0 in established tumors at an average size of 102 mm³. Treatments were administered on days 0, 3, 6, 9, 12 and 15 (n=10/group). FIG. 42B illustrates Survival proportions upon cocktail mRNA-LNPs treatment.

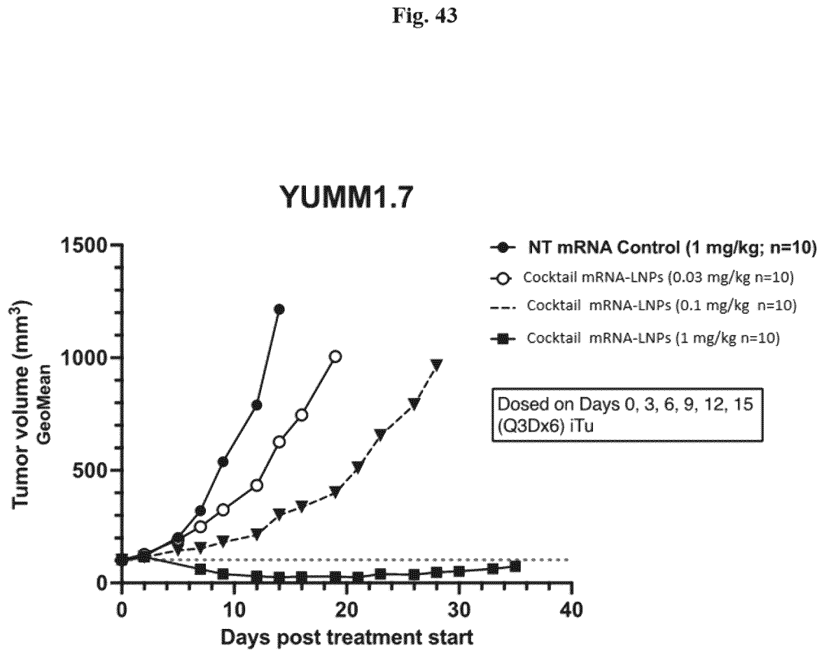

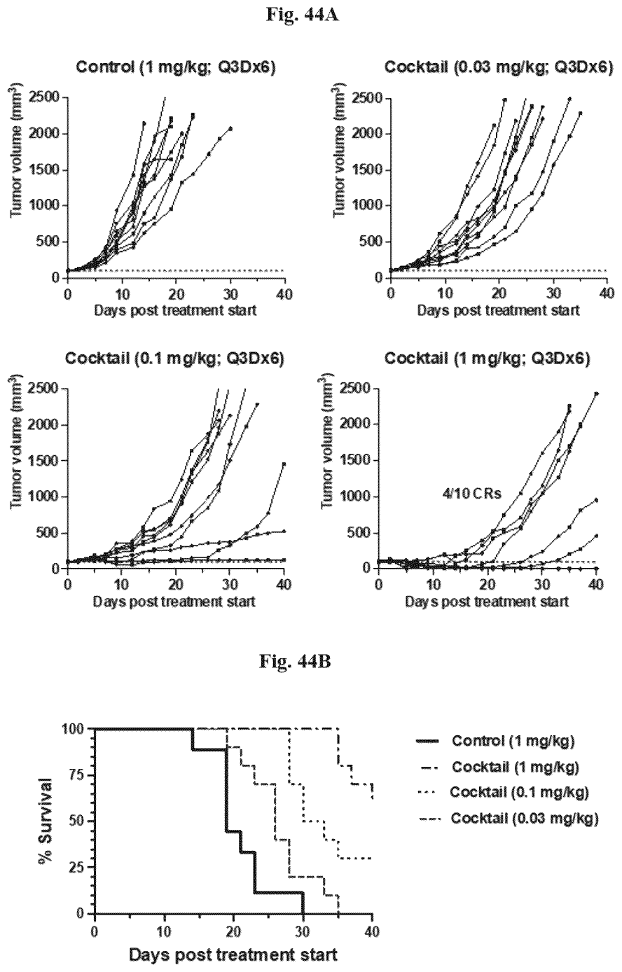

FIG. 43 illustrates treatment of subcutaneous GEMM-derived YUMM1.7 melanoma tumors (BrafV600E, Pten−/−, Cdkn2−/−) with Cocktail mRNA-LNPs (Pro006, Pro045, Pro009, Pro010) vs Pro040 control. Intratumoral injections started on day 0 in established tumors (Q3Dx6). 106 cells were implanted subcutaneously into C57BL/6 mice on day-9. Intratumoral injections started on day 0 in established randomized tumors at an average size of 103 mm³. Treatments were administered on days 0, 3, 6, 9, 12 and 15 (n=10/group). FIG. 44A illustrates individual YUMM1.7 tumor growth curves. Dose titration treatment of subcutaneous YUMM1.7 syngeneic melanoma tumors with cocktail mRNA LNPs (Pro006, Pro045, Pro009, Pro010) vs Pro040 control. Intratumoral injections started on day 0 in established tumors at an average size of 103 mm³. Treatments were administered on days 0, 3, 6, 9, 12 and 15 (n=10/group). FIG. 44B illustrates survival proportions upon cocktail mRNA LNPs treatment.

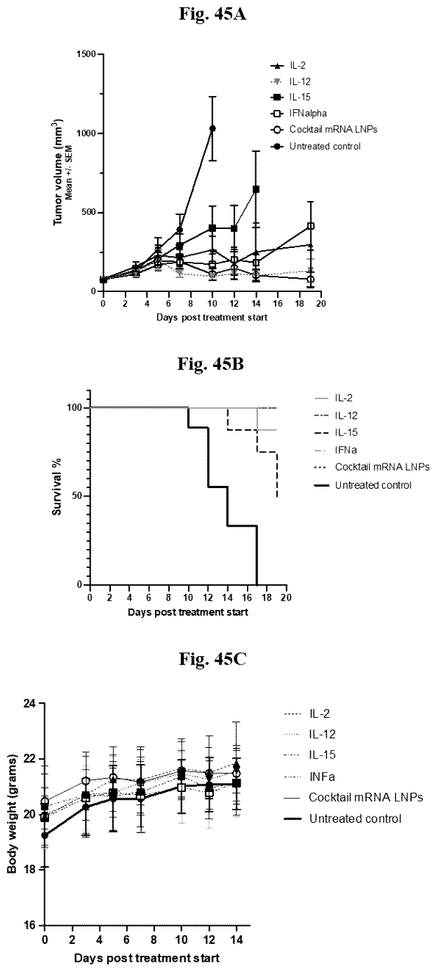

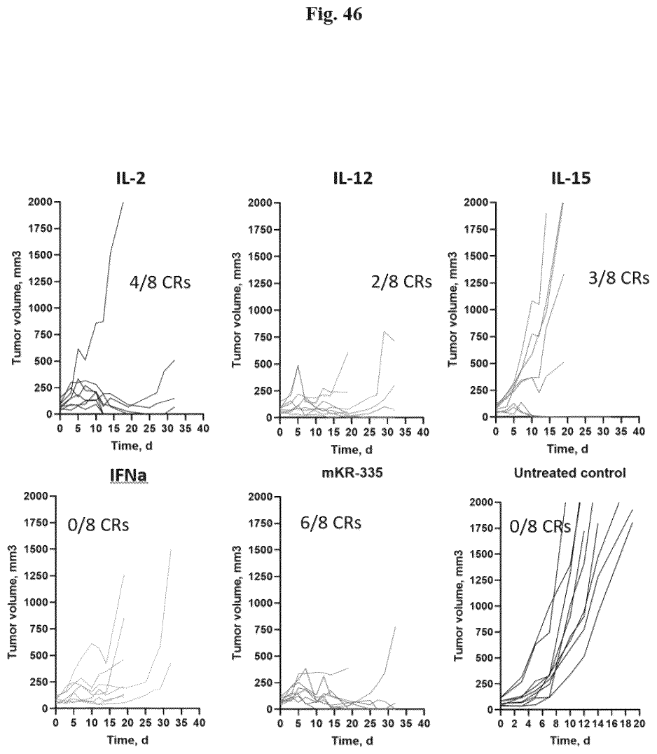

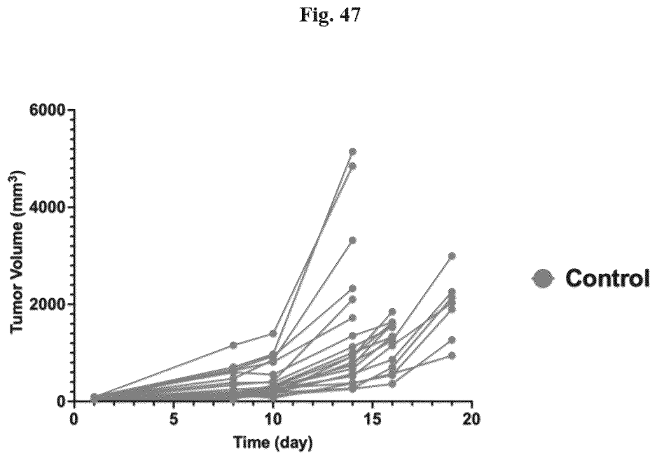

FIGS. 45A-C illustrate cocktail mRNA efficacy evaluation in MC38.K tumors. FIG. 45A illustrates treatment of subcutaneous syngeneic MC38.K (Kerafast) colon tumors Cocktail mRNA-LNPs (1 mg/kg; Pro010, Pro006, Pro011, Pro009) or single cytokine mRNA LNPs (0.25 mg/kg). 5×10⁵ cells were implanted subcutaneously into C57BL/6 mice. Intratumoral injections started on day 0 in established randomized tumors at an average size of 76 mm³. Treatments were administered on days 0, 3, 5, 7, 10 and 12 (n=8/group). FIG. 45B illustrates survival proportions upon cocktail mRNA LNPs or individual treatments. FIG. 45C illustrates body weight measurements (mean+/−SD). FIG. 46 illustrators individual MC38.K tumor growth curves. Intratumoral injections started on day 0 in established tumors at an average size of 76 mm³. Treatments with Pro010, Pro006, Pro011 and Pro009 were administered on days 0, 3, 5, 7, 10 and 12 (n=8/group). FIG. 47 illustrates individual tumor growth curves after implant with 3×10⁵ MC38.K cells in C57BL/6 mice. Re-implanted mice that had CRs following the initial treatment showed no MC38.K tumor growth indicating protective immunological memory formation. Table 5 lists the groups of mice being tested with reimplantation.

TABLE 5
Mice treated with cocktail therapeutic with replantation

| Group | Mice | Model | Previous treatment |
|---|---|---|---|
| 1 | 20 | MC38.K | Treatment naïve |
| 2 | 4 | MC38.K | G1 (Pro010) |
| 3 | 2 | MC38.K | G2 (Pro006) |
| 5 | 3 | MC38.K | G3 (Pro45) |
| 6 | 6 | MC38.K | G5 Cocktail mRNA-LNPs: |
| | | | (Pro006, Pro045, Pro009, Pro010) |

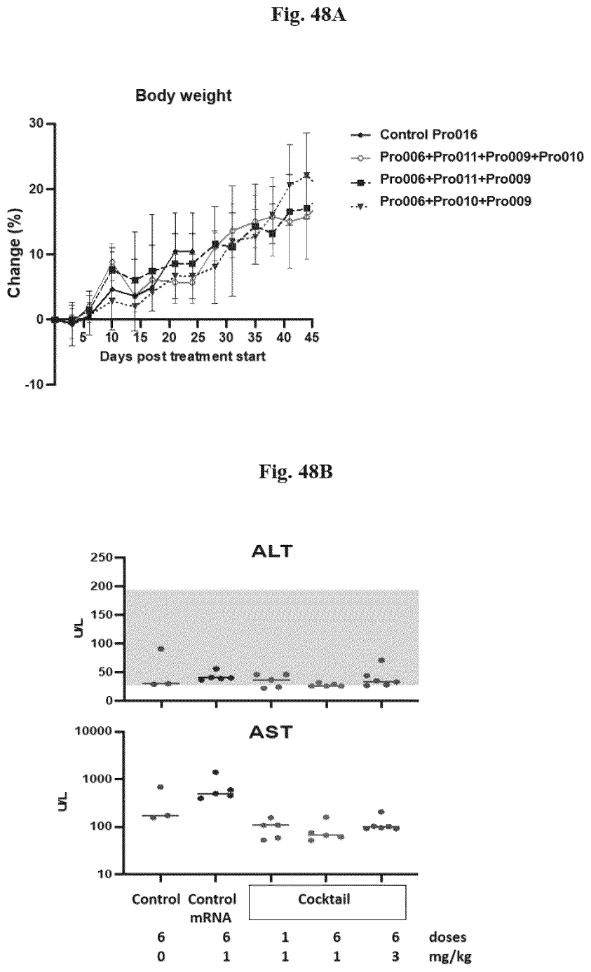

FIGS. 48A-B illustrate tolerability of intratumoral cocktail therapeutic mRNA therapy.

FIG. 48A. illustrates percentage body weight changes after Q3Dx6 intratumoral B16-F10 dosing with a total of 20 mg (˜1 mg/kg) control mRNA or Cocktail mRNA-LNPs LNPs (mean+/−SD). FIG. 48B illustrates liver function test: Normal aspartate transaminase (AST) and alanine transaminase (ALT) serum levels 24 hours after a single or six doses with cocktail.

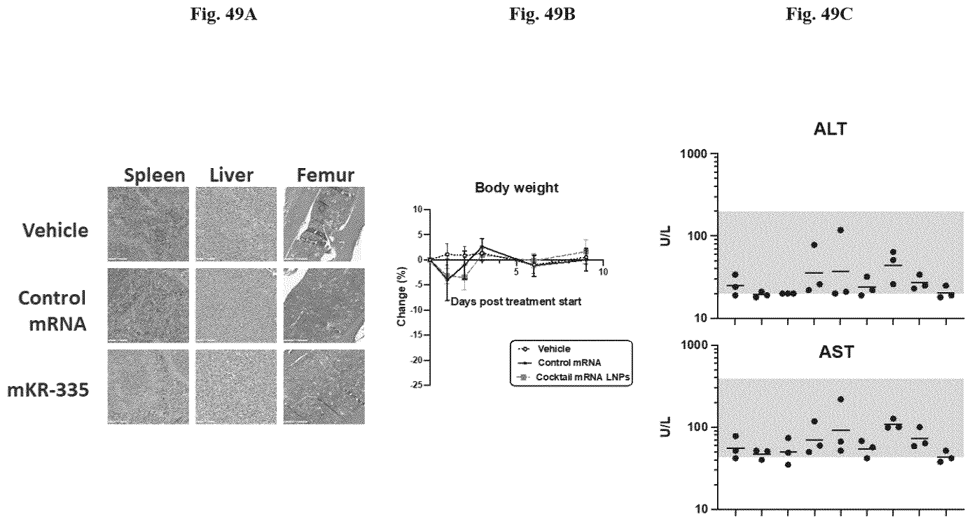

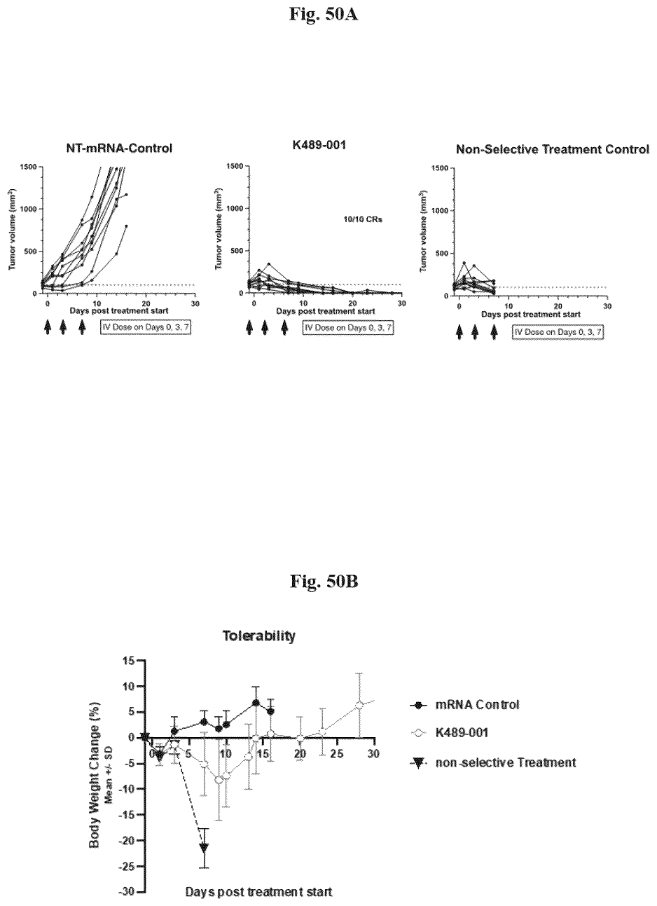

FIGS. 49A-C illustrate tolerability of systemic LNP administration. Female C57BL/6NCrl mice were treated subcutaneously with 1 mg/kg control mRNA LNPs or cocktail therapeutic LMPs. FIG. 49A. H&E sections 24 hours post single dose. FIG. 49B. Percentage body weight changes during a Q3Dx4 dosing regimen (mean+/−SD). FIG. 49C. Liver function test: Normal AST and ALT serum levels 24 hours after a single or four doses, and 7 days after last dose with cocktail therapeutic. Histopathological examination of animals treated subcutaneously with cocktail therapeutic showed no significant changes in liver, spleen, or bone marrows of these animals. In addition, no liver transaminase enzyme elevation was detected in the sera of animals treated with cocktail therapeutic, indicating tolerability to systemic LNP administration of cocktail mRNA LNP. FIGS. 50A-B illustrate K489-001 systemic Administration of oncoselective therapeutic. FIG. 50A illustrates individual MC38.K tumor growth curves after systemic delivery of onco-selective (Pro034) with improved tolerability vs. non-selective treatment (Pro037). 0.25 mg/kg treatment doses were administered intravenously. FIG. 50B illustrates. Body weight changes after treatment start. mRNA control: Pro040, K464-001; or Pro034, K489-001. Non-selective control: Pro037, K479-001.

### Example 8. Oncoselectivity with Masked-Cytokine Constructs

Masked cytokines have blocked receptor-binding domains, which can be removed by tumor-specific enzymes, rendering active cytokines, locally. Masked cytokines would be inactive and safe in the circulation, avoiding damaging side-effects. As shown in FIG. 51 and FIG. 52, IL-12 (either single-chain or two-chain) can be engineered to include a masking domain.

Experimental approaches for measuring levels and activity of Masked cytokine included B16F10 transfected with various mRNA constructs. Culture media was collected for analysis

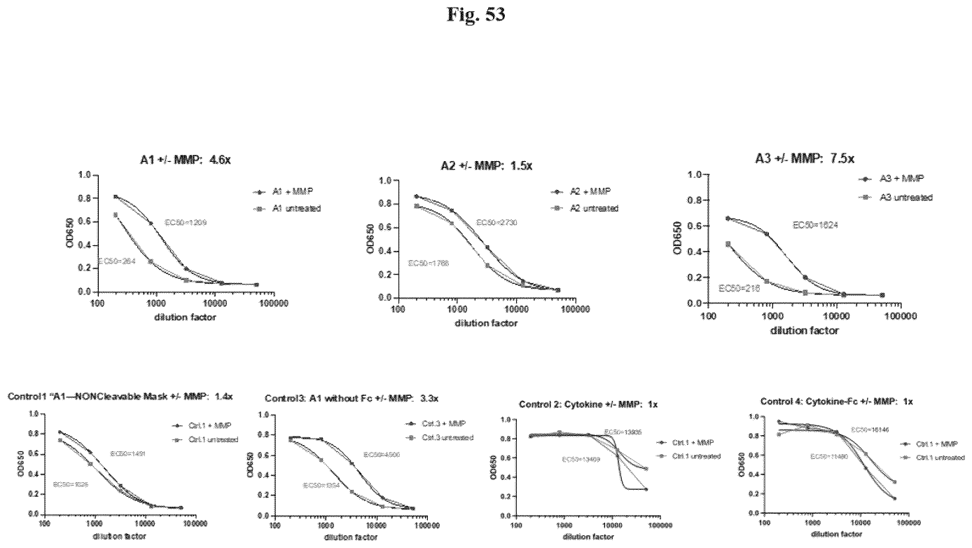

MMP2 enzyme was used to cleave the masked domain in vitro. Cleaved or untreated samples were analyzed ELISA and HEK-reporter cells. FIG. 53 illustrates. EC₅₀ values of masked and protease-treated (MMP) cytokine constructs in a HEK-Blue IL-12 cell reporter assay (Invivogen; hkb-IL-12).

### Example 9. In Vitro Assessment

Example 9 illustrates masked cytokine data relating to the therapeutic described herein.

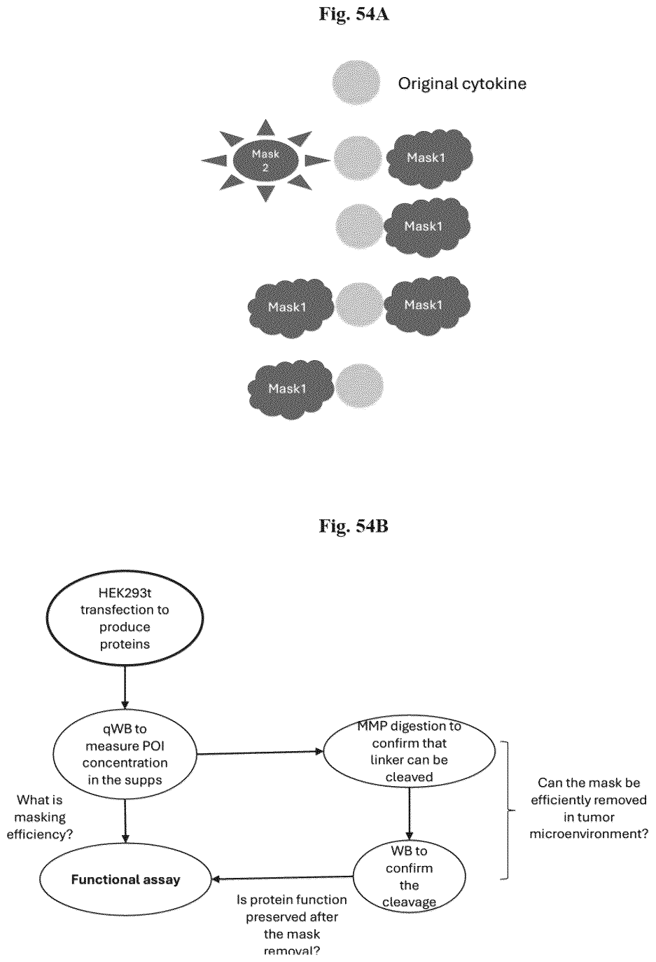

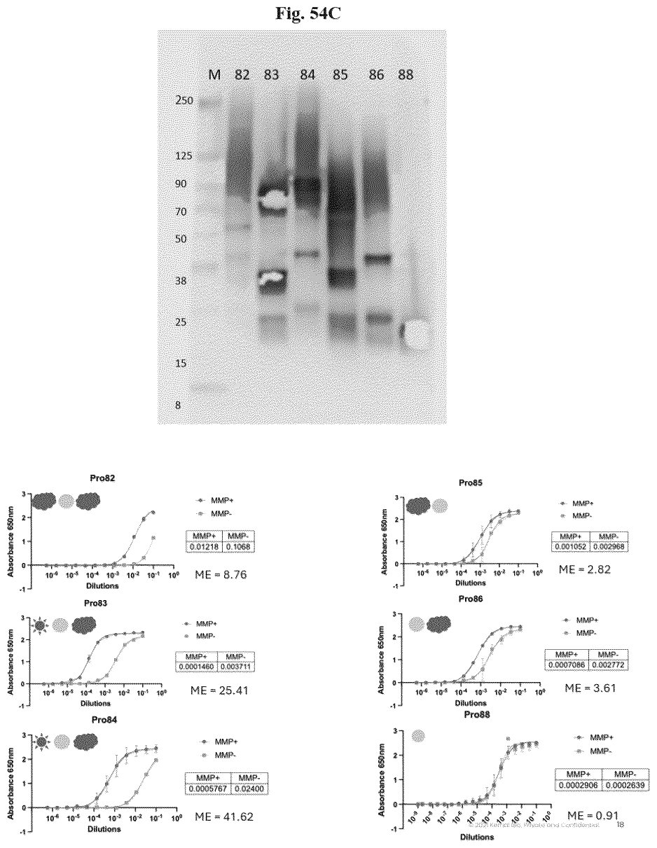

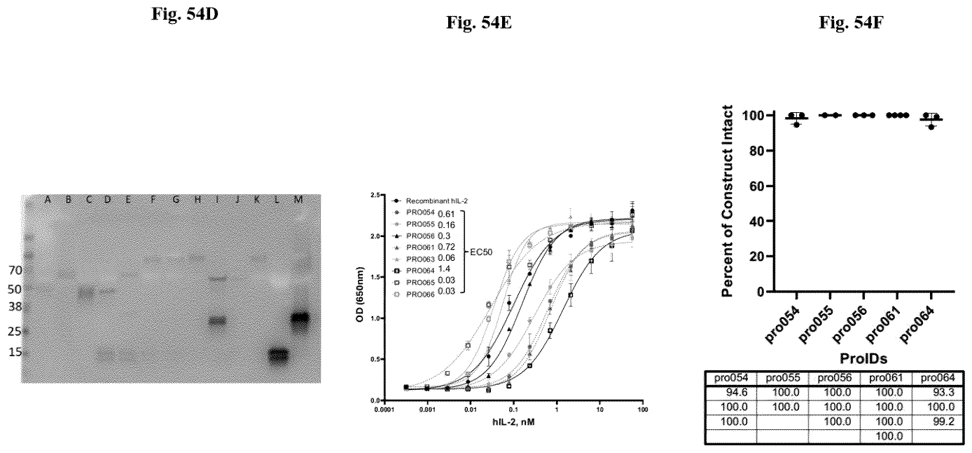

FIG. 54A and FIG. 54B illustrate the design process for the masked cytokine. HEK293t cells were transfected with RNA using Lipofectamin MessengerMAtransfection reagent (Thermo Fisher). One hour later, cell media was changed to serum free media to decrease amount of serum proteins in the samples. 24h after transfection conditional cell media was collected, passed through 0.45 nm filter and then concentrated using 10-30k centrifugal Amicon units. Concentrated supernatants were aliquoted and stored at −20° C. An aliquot of the sample was used for quantitative Western blot or WB (reducing conditions) to determine sample concentration, Membranes were imaged with an Odyssey-M from (Li—COR). Table 14 illustrates monoclonal antibodies for WB analyses.

TABLE 14
Therapeutic tested

| | Mouse | Human |
|---|---|---|
| IL-2 | GeneTex (GTX10753, goat | Anti-IL-2 rabbit mAb (Cell signaling technologies, |
| | | cat# 122395), 1:1000 |
| IL-12 | Sinobio IL-12A Antibody, Rabbit PAb, | Sinobio IL-12A 10021-RPO2, Rabbit (1:1000) |
| p35 | 100321-T32 (1:2000) | |
| IL-12 | Sinobio IL-12B Antibody, Mouse | Invitrogen; IL-12 p40 pAb; ref: PA5-79462; lot: |
| p40 | MAb, 51004-MM09 (1 ug/ml) | YG3995095; rabbit; 1:2000 dilution |
| IL-15 | Rabbit Pab; Sinobio 100322-T32; lot | Anti-Hil-15 Antibody SinoBio IL-15, rabbit; ref: |
| | HB08JU1319-B (1:1000_{—} | 10360-RPO1; 1:1000 |
| IFN-α | Ray Biotech 144-61591-50 Rabbit anti- | SinoBio Anti-Interferon alpha 2/IFNA2 antibody, |
| | IFNA1, 1:1000 | 1:500 |

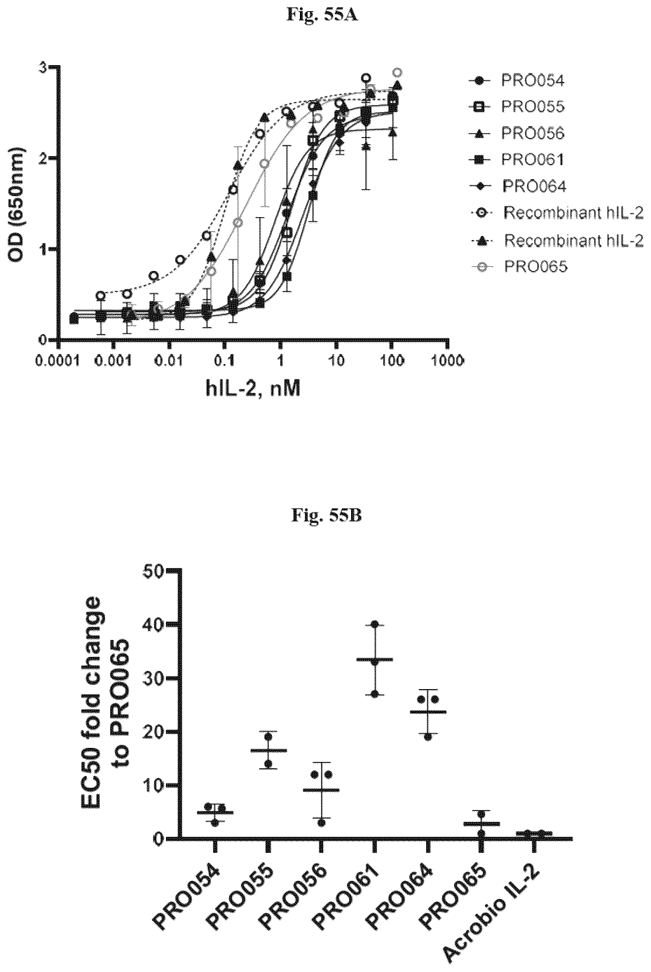

For functional assays cells were cultured according to the manufacturer instructions (Invivogen). For the assay cells were collected without trypsin, counted and plated at 5×104 cells per well Samples were incubated with 50% human serum (37 C, 30 min.) and then added to the reporter cells. 20-24h later SEAP secretion was measured using QUANTI-Blu Solution. Functional assay were run with the following cells lines: hIL-2:IL-2 & IL-15 Reporter HEK 293 Cells (hkb-IL-2, Invivogen); hIL-12: IL-12 Reporter HEK 293 Cells (hkb-IL-12, Invivogen); mIL-12: IL-12 Reporter HEK 293 Cells (hkb-IL-12, Invivogen); hIL-15: IL-2 & IL-15 Reporter HEK 293 Cells (hkb-IL-2, Invivogen); mIL-15: CTLL2 (ATCC); hIFNa: IFN-α/β Reporter HEK 293 Cells (hkb-ifnabv2, Invivogen); or mIFNa: B16-Blu IFN-α/β Cells (bb-ifnt1, Invivogen). FIG. 54C illustrates construct mRNAs transfected into HEK293t cells using Lipofectamine Messenger Max. Supernatants were collected in 24 hours and concentrated. 3 ul of sample was used to run SDS-PAGE. All chimeric proteins show additional bands which might represent cleaved proteins. FIG. 54D illustrates translation of hIL-2 entities A to M from mRNAs. SDS-PAGE and WB analysis of in vitro translated hIL-2 entities stained with anti-hIL-2 mAb (CST,12239S). L: mRNA encoding free human IL-2. Proteases present in HEK cell culture caused partial linker cleavage for constructs D, E, F and G. FIG. 54E illustrates masking efficiency evaluation by determining EC50 values in a functional assay for hIL-2 entities using HEK-Blue-IL-2 reporter cell line. The activation of the JAK-STAT pathway was monitored as a dose-response to hIL-2 protein entities translated from mRNAs A-M. Cells were stimulated with increasing concentrations. After overnight incubation, the STAT5 response was determined using QUANTI-Blue Solution, a SEAP detection reagent, and reading the optical density (OD) at 650 nm. N=2 (technical replicates), Mean±SD. Unmasked construct L was aligned with the standard free recombinant hIL-2. Constructs H and K had the highest EC50. FIG. 54F illustrates percentage of intact (uncleaved) protein in HEK cell supernatant for hIL-2 entities was determined by WB analysis. For the entities a high level of protein integrity and low level of linker, cleavage was detected. Integrity was determined for each entity with 2 to 4 transfections with mRNAs (table). Mean+/−SD was shown. FIG. 55A and FIG. 55B illustrate masking efficiency evaluation by determining EC50 values in a functional assay for hIL-2 entities using HEK-Blue-IL-2 reporter cell line. Activation of IL-2 signaling was observed in presence of increasing concentrations of IL-2 entities. FIG. 55A. Mean±SD of two to three different transfection reactions for each entity. FIG. 55B. Masking efficiency as fold EC50 increase relative to Pro065 (Mean±SD).

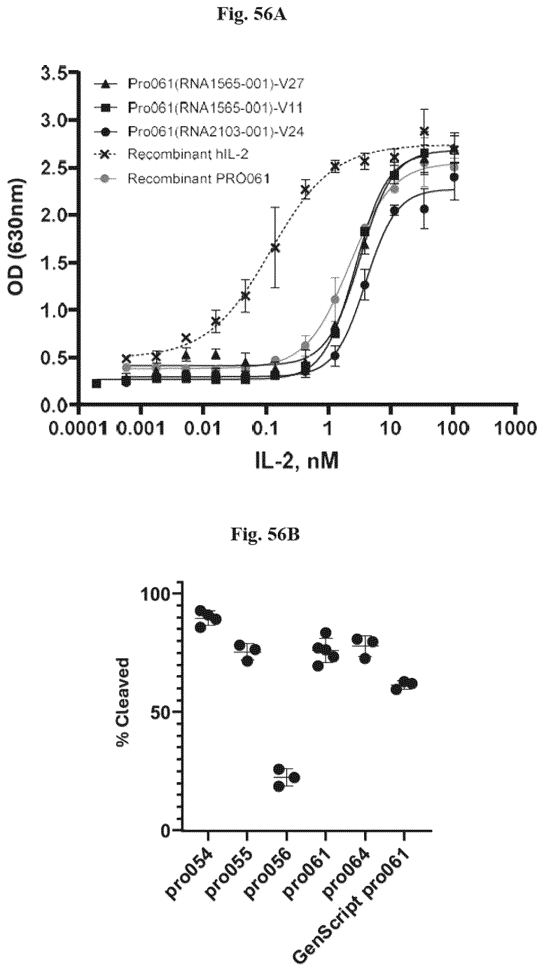

FIG. 56A illustrates comparison of functionality and masking efficiency of hIL-2 Pro061 from supernatants of different mRNA batches vs purified protein (Protein A followed by size exclusion columns; Genscript Biotech). Activity and masking efficiency of Pro061 produced from mRNAs correlated with the plasmid based Pro061 purified at GenScript. FIG. 56B illustrates validation that linkers in IL-2 entities were cleavable by incubation with activated MMP-2 protease. Percentage of cleaved protein was determined using WB. Pro054, Pro055, Pro061, and Pro064 were sensitive to MMP-2 digestion and protease-dependent removal of masking and half-life extension domains.

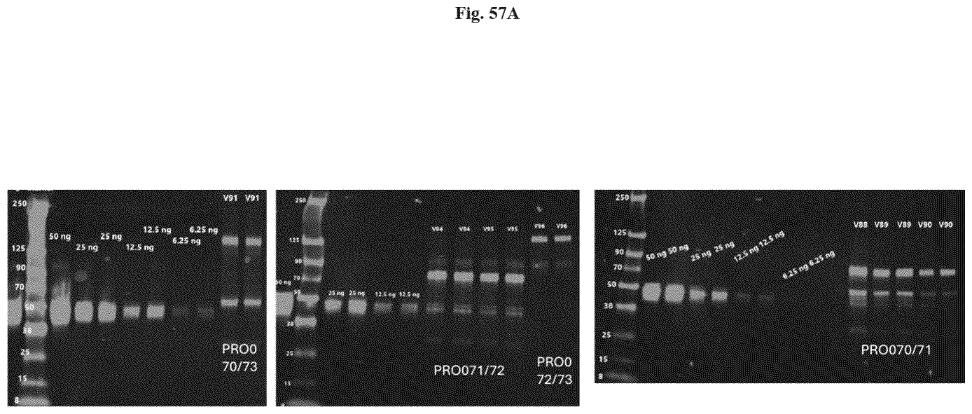

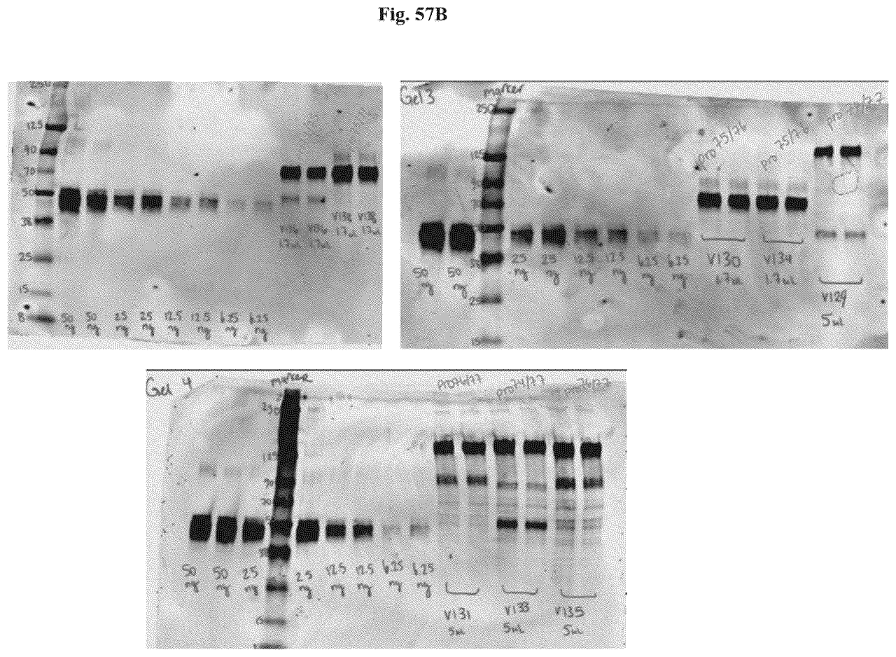

FIG. 57A illustrates IL-12 cleavable protein entities produced in vitro from their respective mRNAs. (reducing SDS-PAGE). HEK293t cells were transfected with RNA using Lipofectamine MessengerMAX. The protein weights and integrity was analyzed using quantitative Western Blot. Membranes were developed with anti-IL-12B (p40) mAb to detect PRO071 or PR0073. Predicted MW for PRO071 is 69 kDa and 108 kDa for PR0073. Proteins may run higher due to glycosylation. FIG. 57B illustrates mIL-12 non-cleavable protein entities produced in vitro from their respective mRNAs. (reducing SDS-PAGE). HEK293t cells were transfected with RNA using Lipofectamin MessengerMAX Transfection Reagent. The protein weights and integrity was analyzed using quantitative Western Blot. Membranes were stained with anti-IL-12B mAb to detect Pro075 and Pro077. Predicted MW for Pro075 was 65 kDa, and for Pro077 was 100 kDa. Proteins could run higher due to glycosylation.

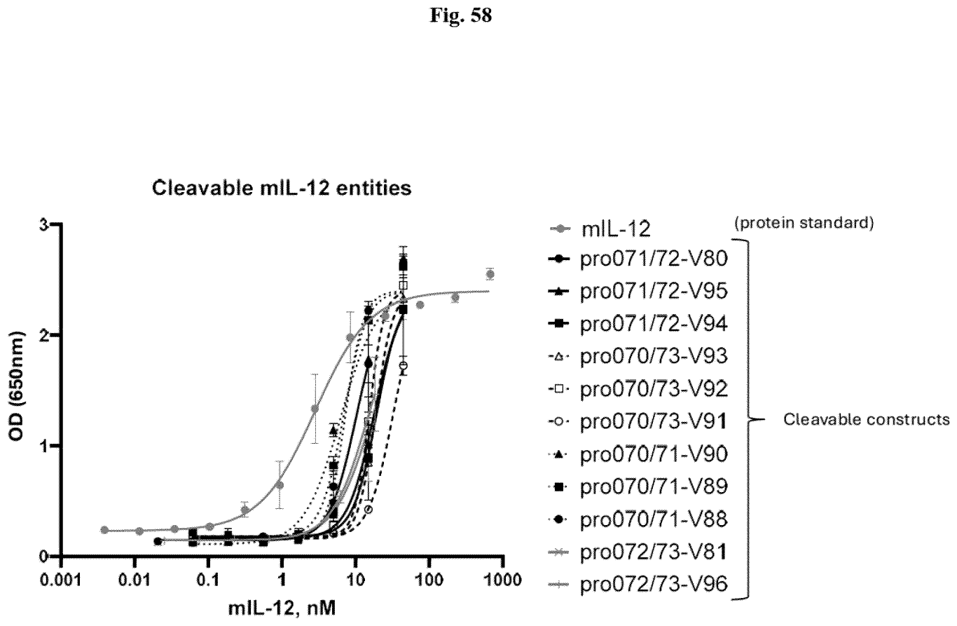

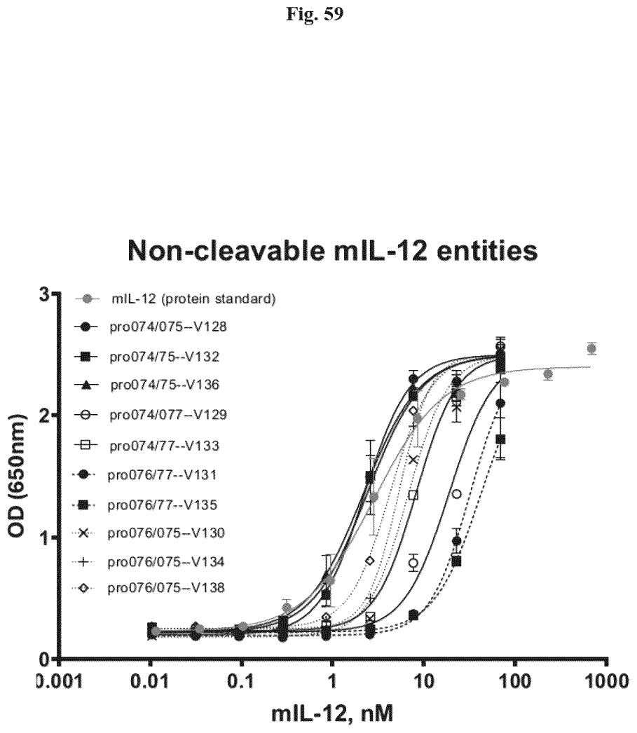

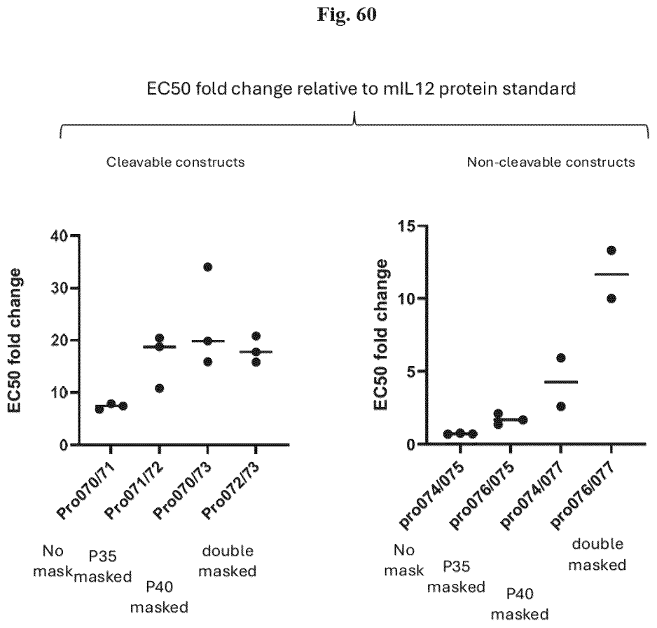

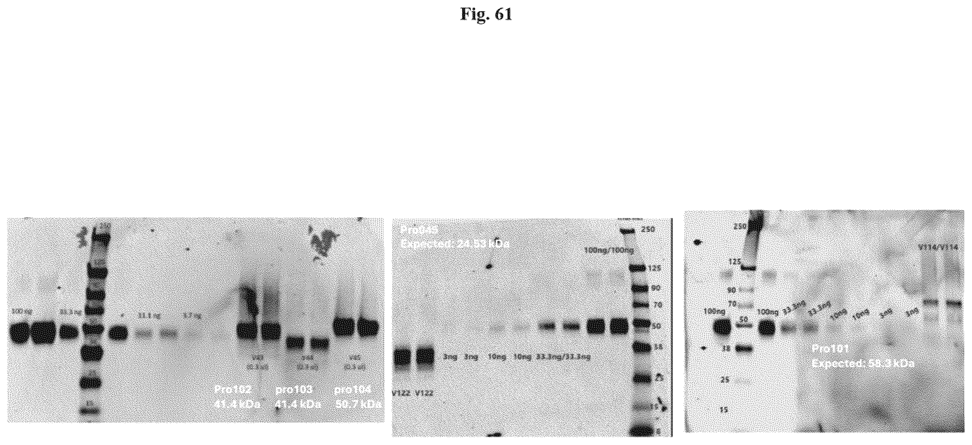

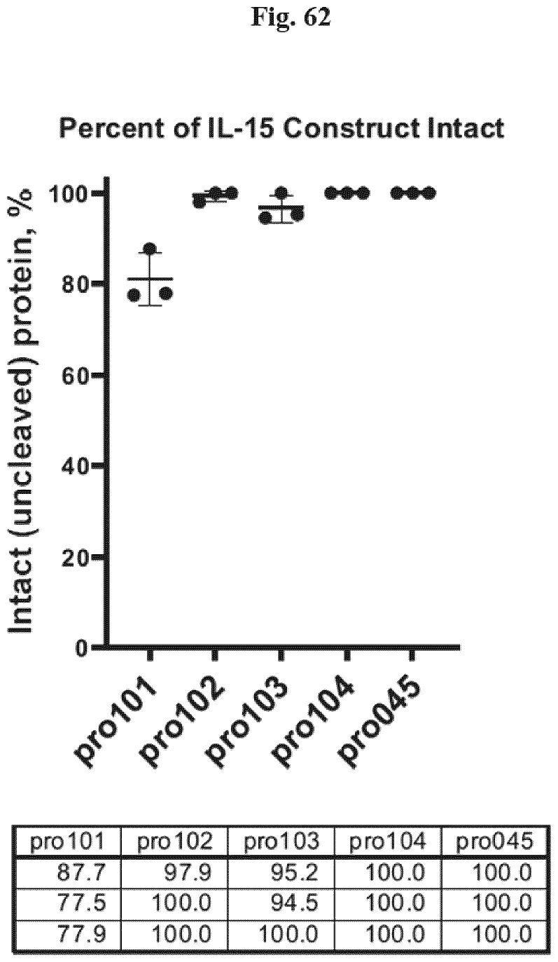

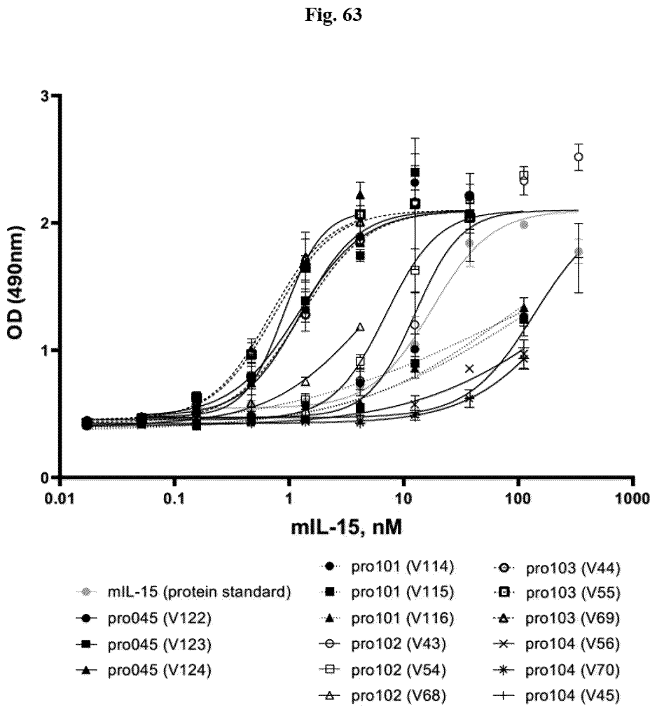

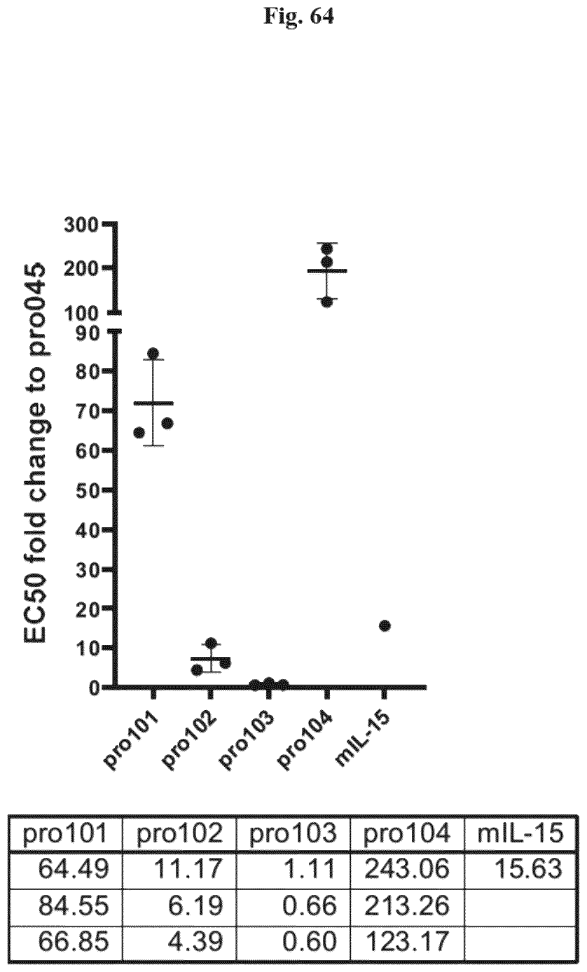

FIG. 58 illustrates masking efficiency evaluation by determining EC50 values in a functional assay for cleavable hIL-12 entities using HEK-Blue-IL-12 reporter cell line. Activation of IL-12 signaling was observed in presence of increasing concentrations of IL-12 entities. Mean t SD of two to three different transfection reactions for each entity. FIG. 59 illustrates masking efficiency evaluation by determining EC50 values in a functional assay for non-cleavable hIL-12 entities using HEK-Blue-IL-12 reporter cell line. Activation of IL-12 signaling was observed in presence of increasing concentrations of IL-12 entities. Mean+SD of two to three different transfection reactions for each entity. FIG. 60 illustrates masking efficiency as fold EC50 increase relative to Mil-12 protein standard (Mean±SD). FIG. 61 illustrates mIL-15 protein entities produced in vitro from their respective mRNAs. Predicted sizes in kDa were provided. (glycosylated, reducing SDS-PAGE). HEK293t cells were transfected with RNA using Lipofectamin MessengerMAX Transfection Reagent. The protein weights and integrity was analyzed using quantitative Western Blot. FIG. 62 illustrates percentage of total intact (e.g., uncleaved) protein of mIL-15 entities in supernatant. For each individual IL-15 entity, results from 3 mRNA transfections were shown. FIG. 63 illustrates cell survival after addition of different concentrations of mIL-15 entities. N=2, technical replicates. Mean±SD. Using CTLL2 cells, cells were preincubated in cell media with low IL-2. Then, samples were applied and left to incubate for 3 days. Before that, samples were preincubated with 50% human serum for 30 min. Cell survival was measured using Promega Substrate Cell Titer 96 Aqueous One Solution Reagent. For each individual IL-15 entity results from 3 mRNA transfections are shown. FIG. 64 illustrates fold change of EC50 for IL-15 entities calculated relative to unmasked entity (PRO045). EC50s were calculated using the functional CTLL2 assay in FIG. 63. Mean+/−SD of 3 transfection replicates with mRNAs are shown. Treatment with Pro101 led to higher EC50 fold change relative to Pro045 compared to Pro102. Despite partial cleavage observed in Pro101 and no cleavage in Pro102. Pro104 and Pro101 (both contain 2 HSA binding domains) demonstrated the highest EC50 fold change.

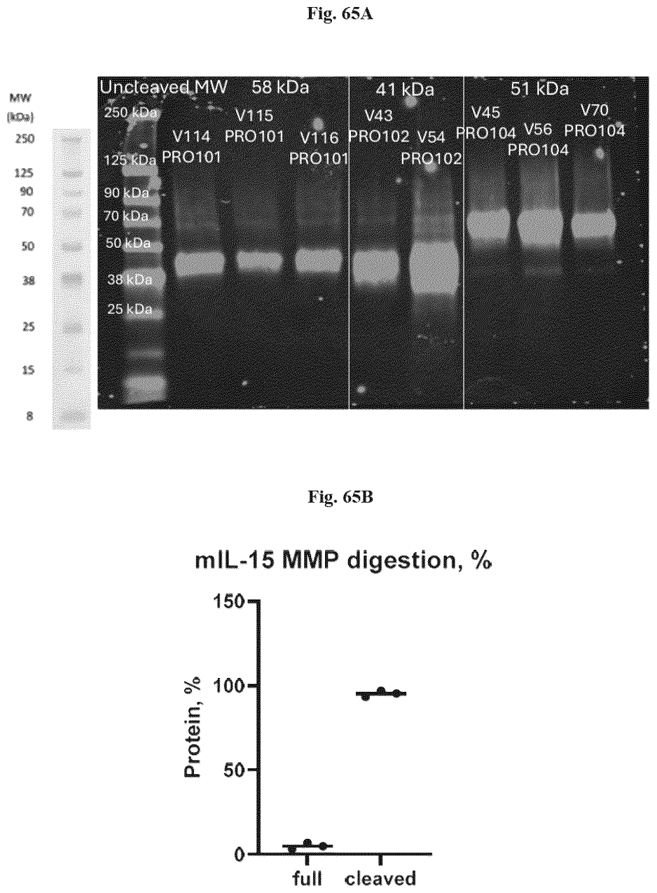

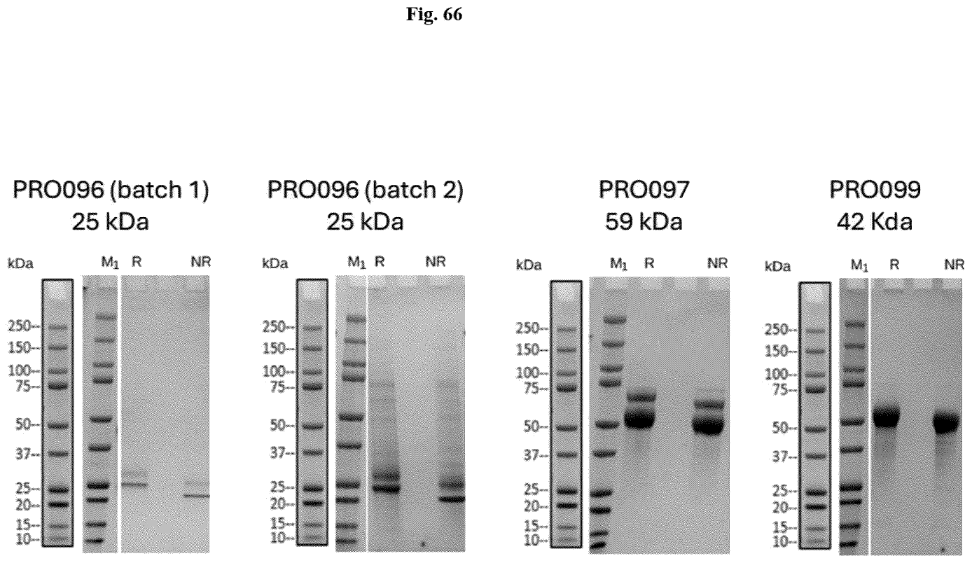

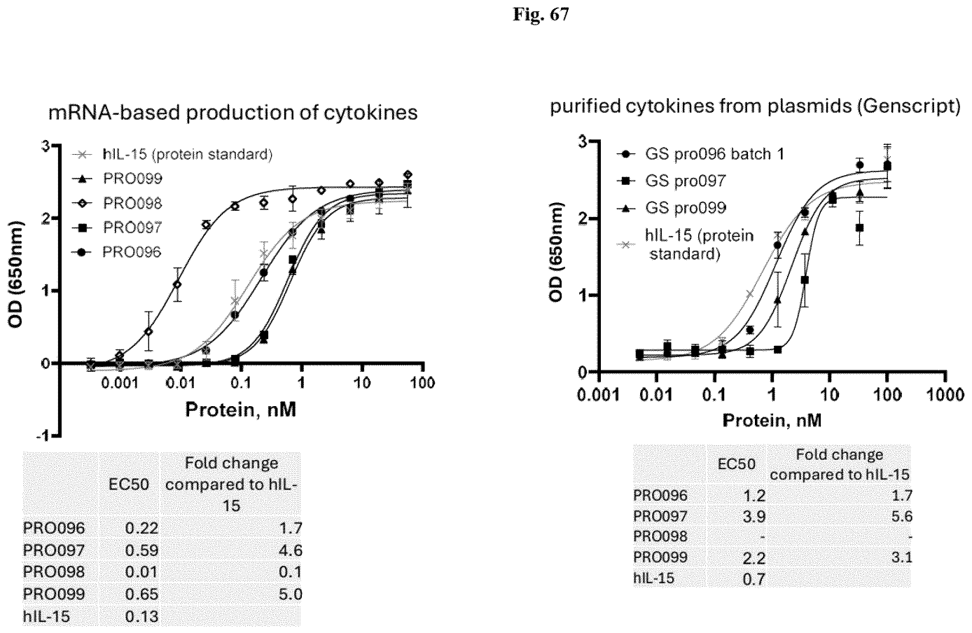

FIG. 65A illustrates protease-dependent cleavage. SDS-PAGE WB results for mIL-15 entities after digestion with MMP2 to validate cleavability of the linkers. Pro104 served as a control with a non-cleavable linker. Fully cleaved product was expected at ˜25 kDa but ran higher mainly due to glycosylation. FIG. 65B illustrates effective linker cleavage in Pro101. Fraction of cleaved Pro101 following MMP2 protease treatment was determined by WB. FIG. 66 illustrates human IL-15 protein entities produced from their respective mRNAs. Predicted sizes in kDa were provided. (glycosylated, reducing SDS-PAGE). SDS-PAGE results for lead mIL-15 candidates were digested with MMP2 to test cleavability of the linkers. Proteins were produced by GenScript. R—reducing conditions; NR—reducing conditions. For the functional assay, HEK-Blue-IL-2 cells were used. Samples were incubated with human serum for 30 min at 37° C. before use. FIG. 67 illustrates IL-2 pathway activation in presence of hIL-15 entities produced from RNA or as a purified protein (size exchange chromatography).

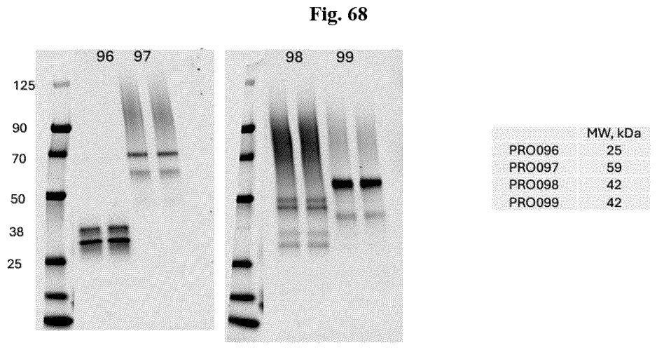

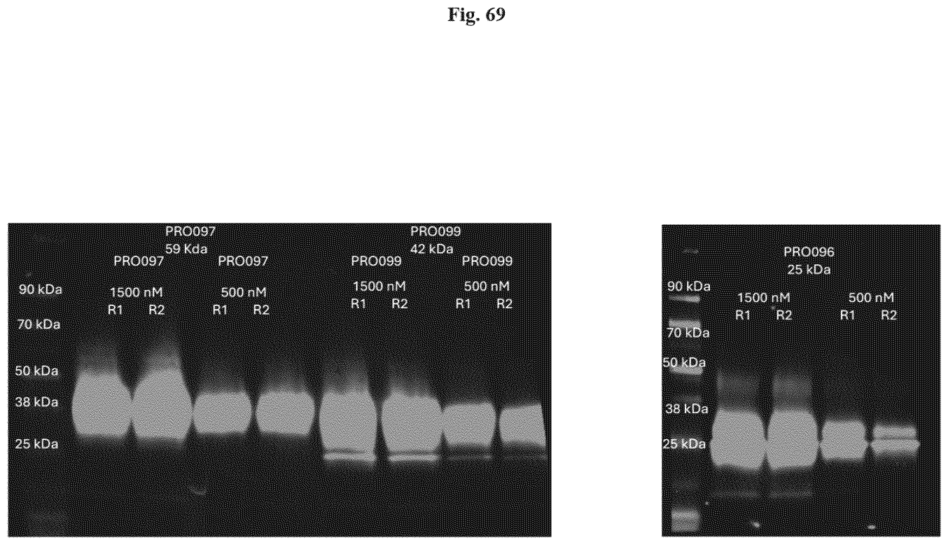

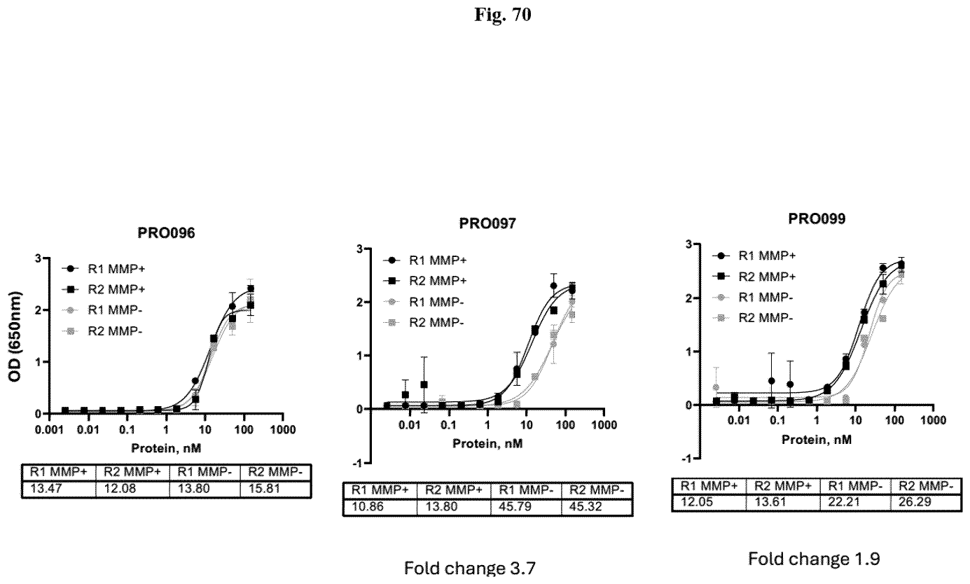

FIG. 68 illustrates hIL-15 protein entities produced in vitro from their respective mRNAs. Predicted sizes in kDa were provided. (glycosylated, reducing SDS-PAGE). HEK293t cells were transfected with RNA using Lipofectamin MessengerMAX Transfection Reagent. The protein weights and integrity was analyzed using quantitative Western Blot. FIG. 69 illustrates protease-dependent cleavage. SDS-PAGE WB results for hIL-15 entities after digestion with MMP2 were shown to validate cleavability of the linkers. Fully cleaved product was expected at ˜25 kDa, but runs higher mainly due to glycosylation. FIG. 70 illustrates comparison of masking efficiency of hIL-15 entities before and after digestion to demonstrate that protein functionality (e.g., ability to activate IL-2 signaling) was preserved after the digestion.

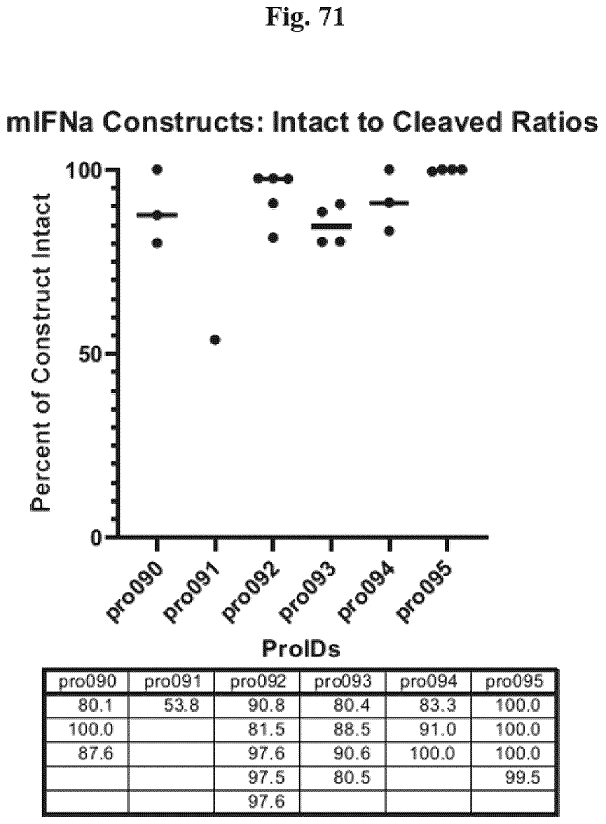

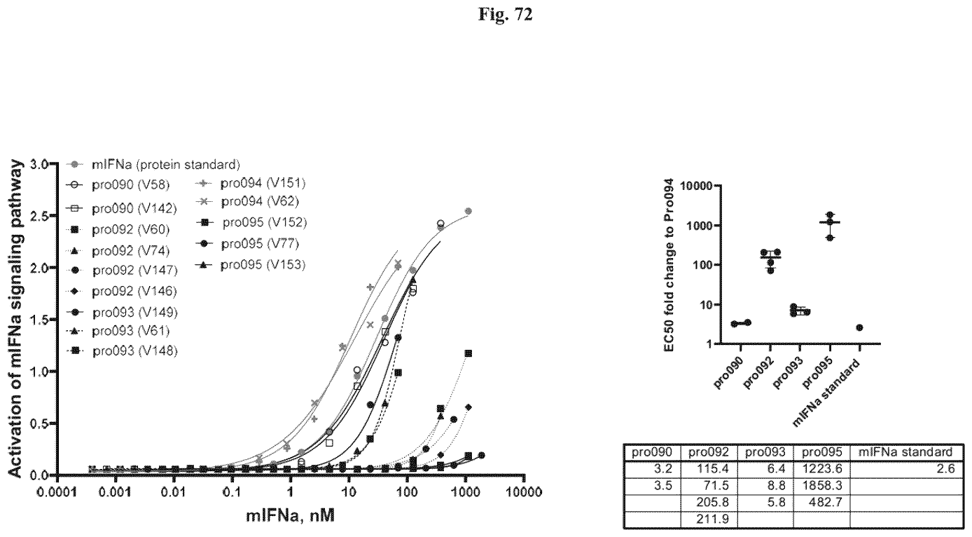

FIG. 71 illustrates percentage of total intact (uncleaved) mIFN-α entities in supernatant. FIG. 72 illustrates activation of mIFN-α signaling after treatment with increasing concentrations of different mIFNa entities. Activity of in-house produced Pro061 correlated with the Pro061 purified by GenScript, which supported validity of developed qWB.

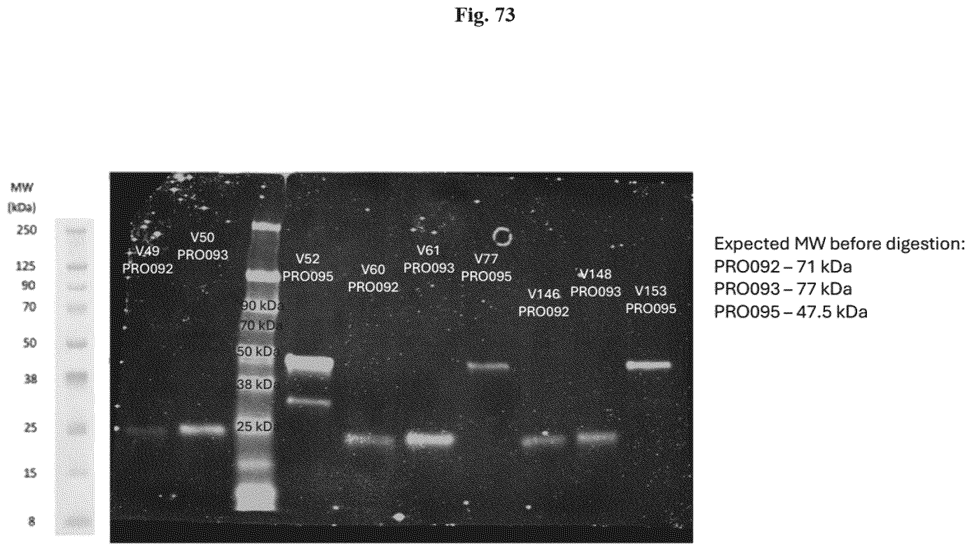

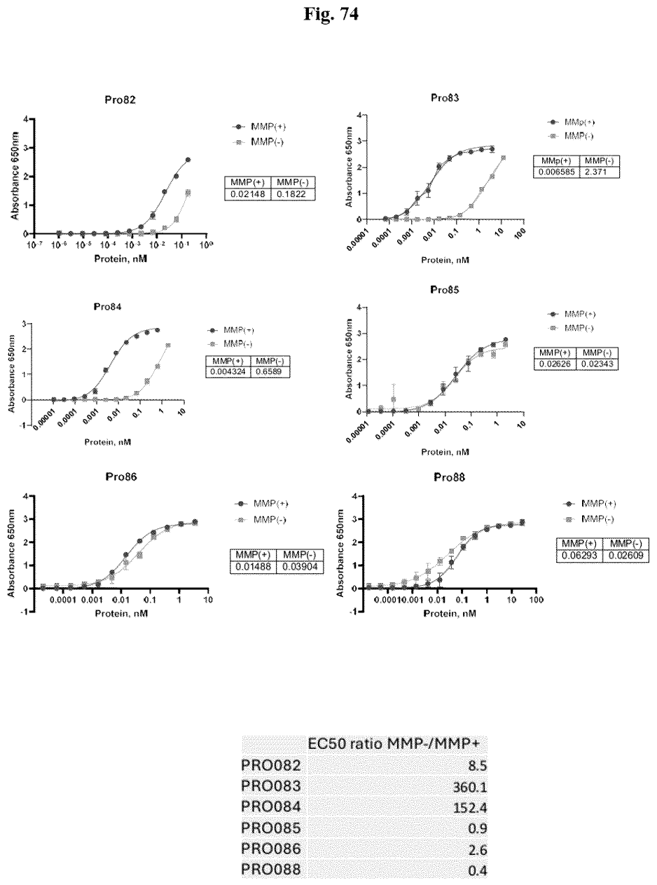

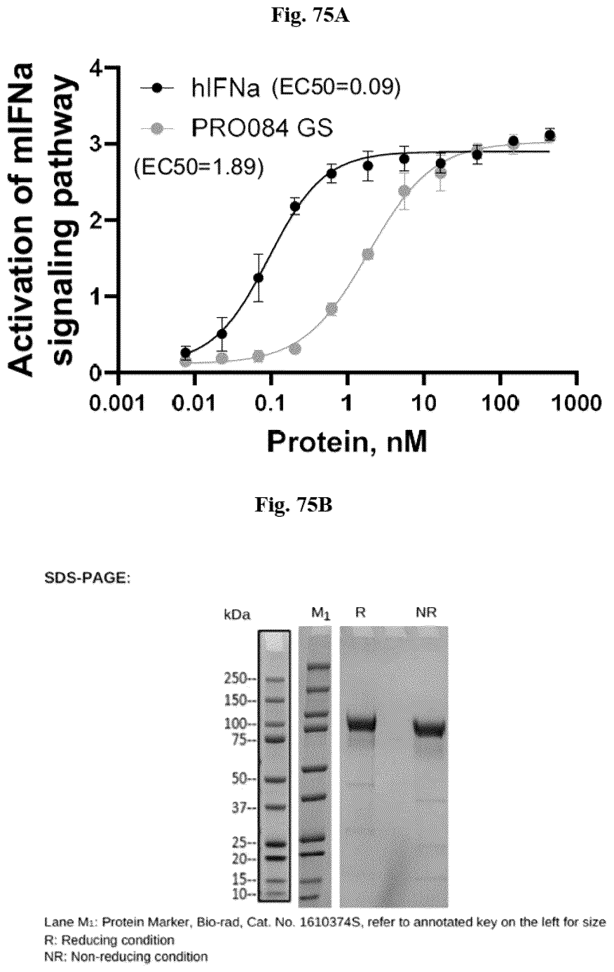

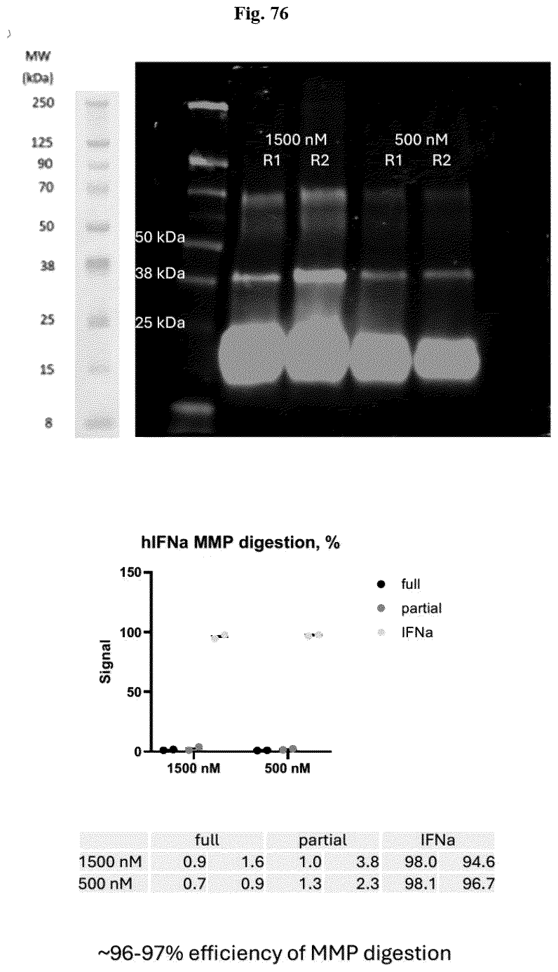

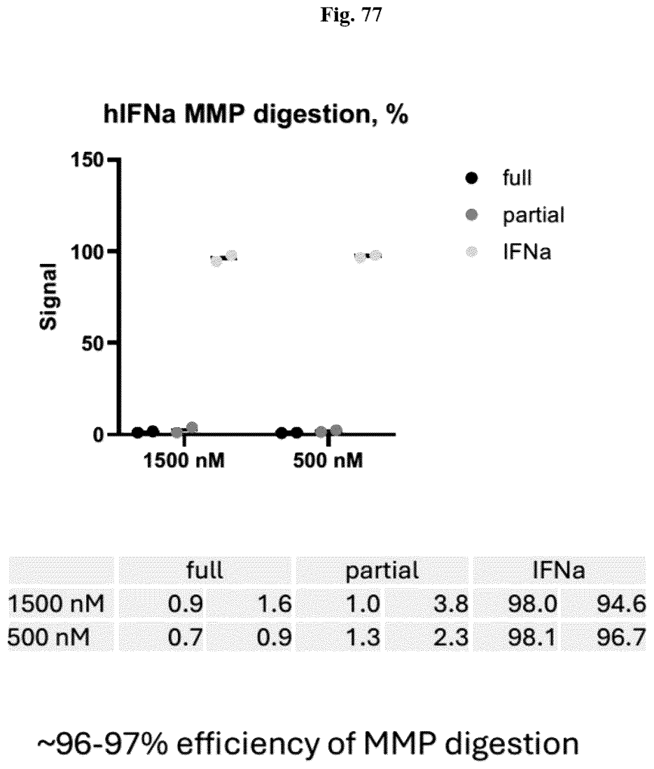

FIG. 73 illustrates protease-dependent cleavage. SDS-PAGE WB results for mINF-α entities after digestion with MMP2 to validate cleavability of the linkers were shown. Fully cleaved product was expected at ˜25 kDa but ran higher mainly due to glycosylation. Pro104 served as a control with a non-cleavable linker. FIG. 74 illustrates comparison of hIFN-α signaling activation after in presence of hIFN-α entities produced from RNA before and after digestion to assess masking efficiency and demonstrated that protein functionality (e.g., ability to activate IFNa signaling) was preserved after the digestion. FIG. 75A illustrates comparison of purified PRO084 (His-tag+SEC) hIFN-α and unmasked hIFN-α (protein standard). FIG. 75B illustrates SDS-PAGE result for purified Pro084. FIG. 76 illustrates percentage of digested protein determined using Wester blotting (WB). FIG. 77 illustrates comparison of masking efficiency of hIFN-α entities before and after digestion to demonstrate that protein functionality (e.g., ability to activate IFNa signaling) was preserved after the digestion. EC50 fold change between MMP+ and MMP− was about 10 times. It was two times lower than for native samples.

### Example 10. In Vivo Assessment

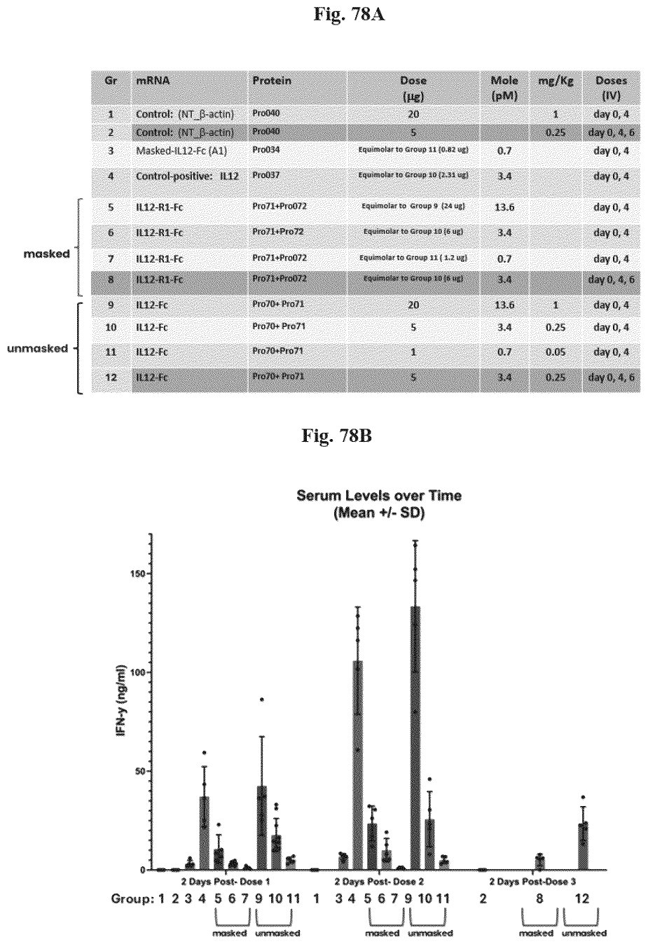

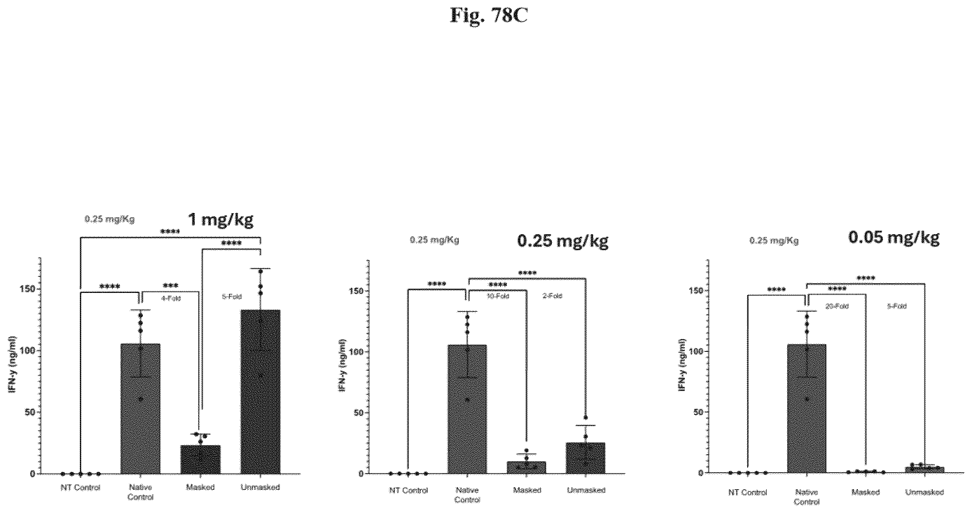

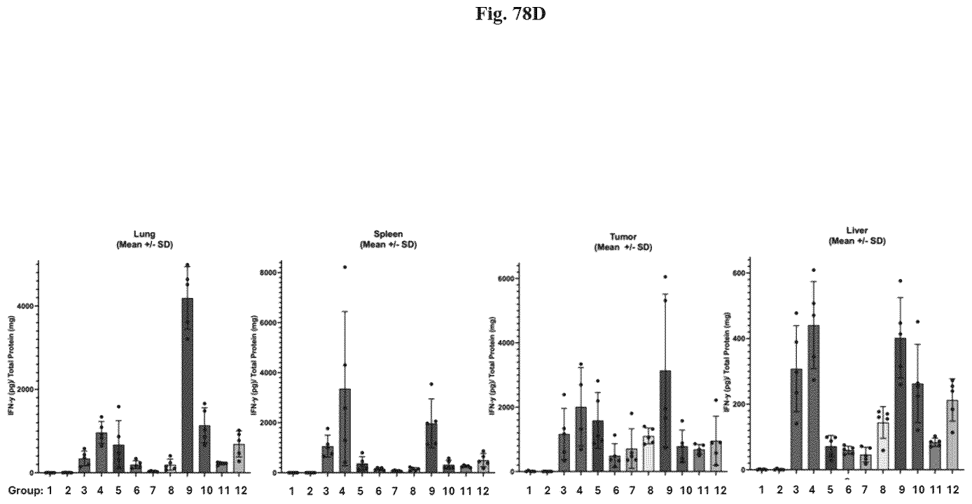

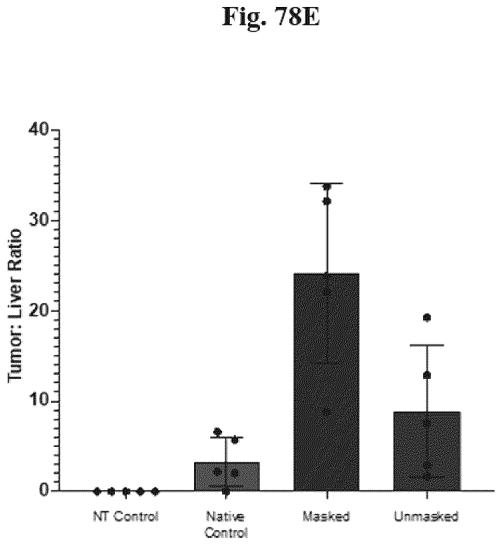

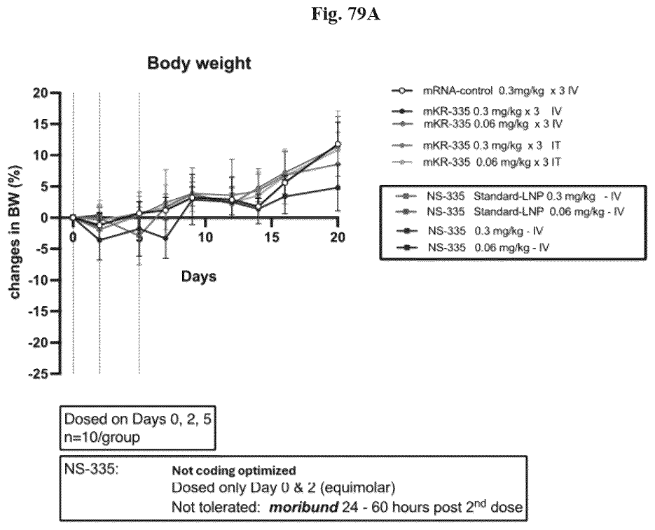

FIG. 78A illustrates evaluation of IFN-γ pharmacodynamic responses (downstream immune activation of mKR-336) to the treatment. Tumor selectively increased IFNgamma over a 20-fold dose range (0.05-1 mg/Kg). Inflammatory milieu induced in tumor (MSD assay of tumor lysates). Dose titration of systemic KR-336/IL-12 mRNA-LNPs for evaluating single-chain masking effect was performed by measuring IFNgamma levels in various organs 48 hours after dosing. Standard mRNA-LNPs were administered IV in C57BL/6J female mice carrying a subcutaneous MC38.K tumors (n=5/group). Unmasked control was encoded by mRNA-LNP in group 4. FIG. 78B illustrates IFNgamma levels in serum following dosing. FIG. 78C illustrates serum IFNgamma 48 hours post-dose 2. “Native” dose at 0.25 mg/kg was a not tolerated control (>MTD) and induced toxic IFNg levels and shown as standard in all three panels. ANOVA statistical analysis. By comparison, treatment with masked entity was tolerated (IFNgamma level, body weight, overall body score) demonstrating an increase in biological tolerability and therapeutic index through masking. FIG. 78D illustrates IFNgamma levels in organ lysates normalized to total protein content (BCA assay based). mKR-336 induced strong IFNy Levels in the tumor while avoiding toxic levels in the liver. FIG. 78E illustrates oncoselectivity of mKR-336. Graph showed the tumor:liver ratio of IFNgamma levels following treatment. As shown in FIG. 78, mKR-335 dosed IT had demonstrated efficacy in the MC38.K murine tumor model.

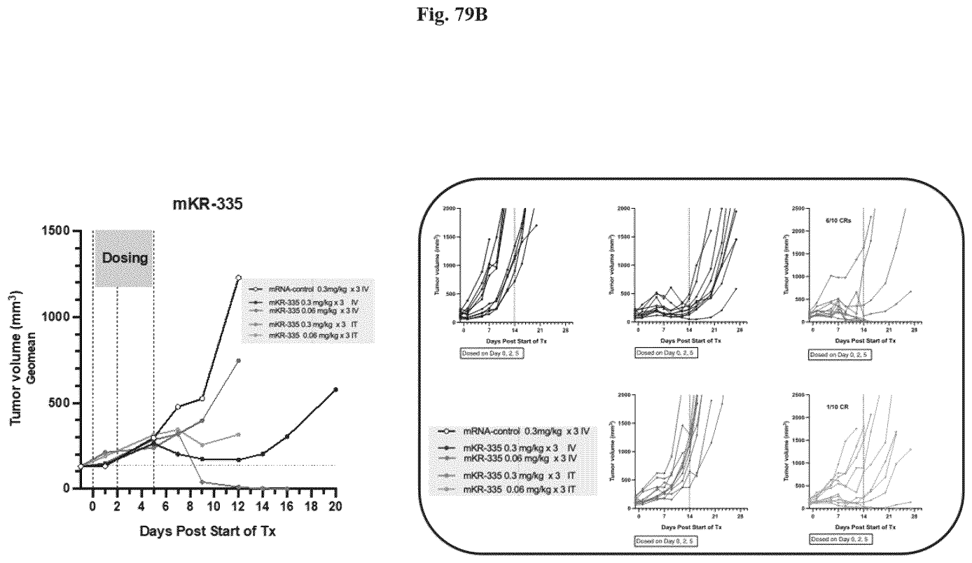

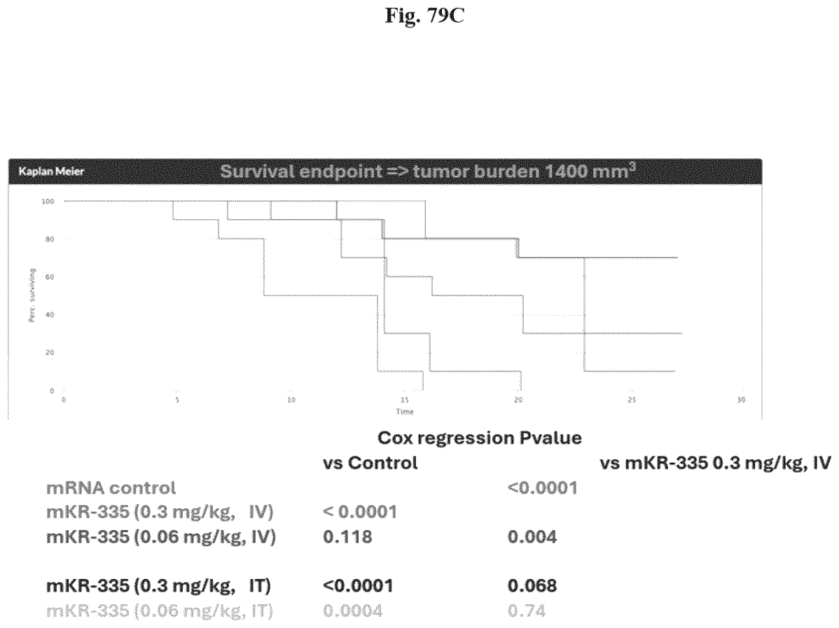

FIG. 79 illustrates efficacy of KR-335/336 when administered systemically. Masking of mKR-335 (Pro064, Pro072, Pro073, Pro101, or Pro093) and mKR-336 (Pro072 or Pro073) for was tested for tolerability and anti-tumor efficacy efficiency vs unmasked control (NS-335: Pro065, Pro037, Pro045, or Pro094; NS-336: Pro070 or Pro07l) in the MC38.K C57BL/6 model. Peripheral mRNA-LNPs were employed and administered IV for systemic administration or IT for comparison with direct tumor application. FIG. 79A illustrates mKR-335 was tolerated when administered IV, while NS-335 was not. FIG. 79B illustrates mKR-335 provided TGI benefit. FIG. 79C illustrates overall survival benefit (tumor burden limit at 1400 mm³).

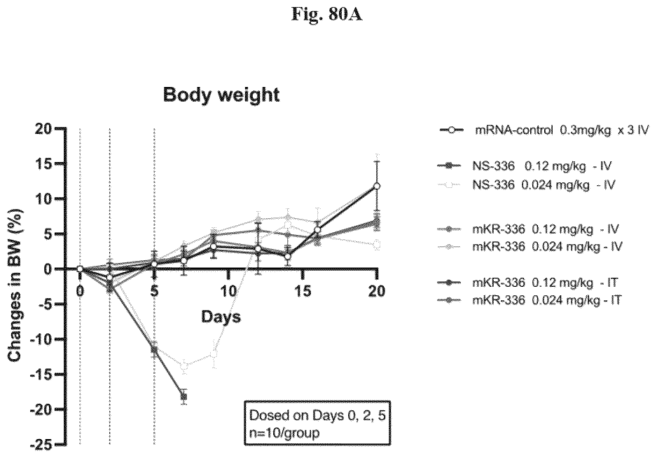

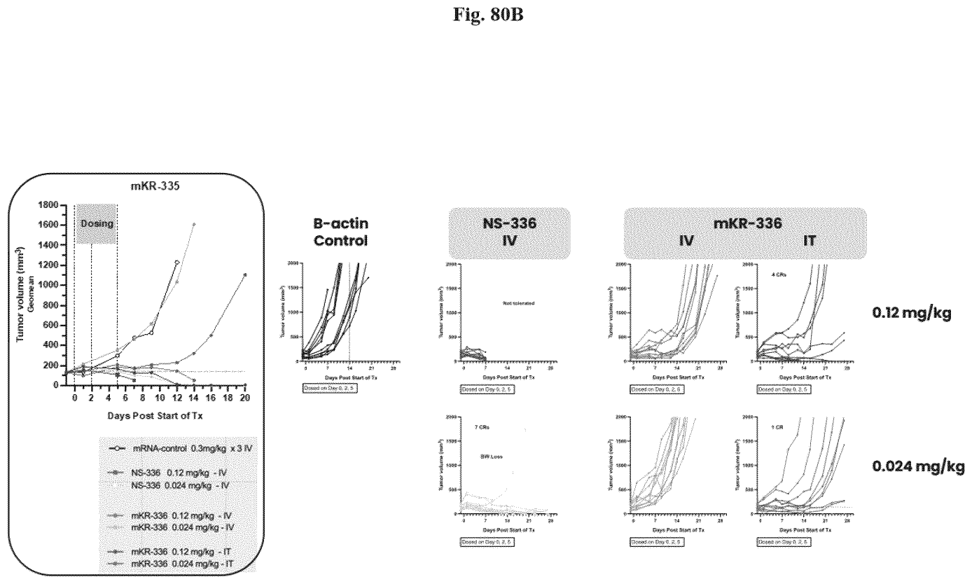

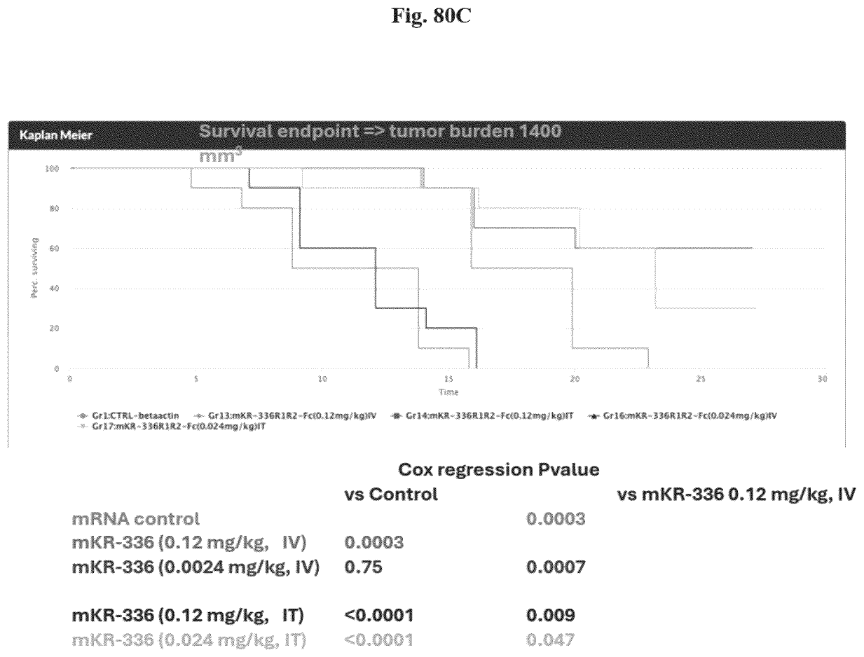

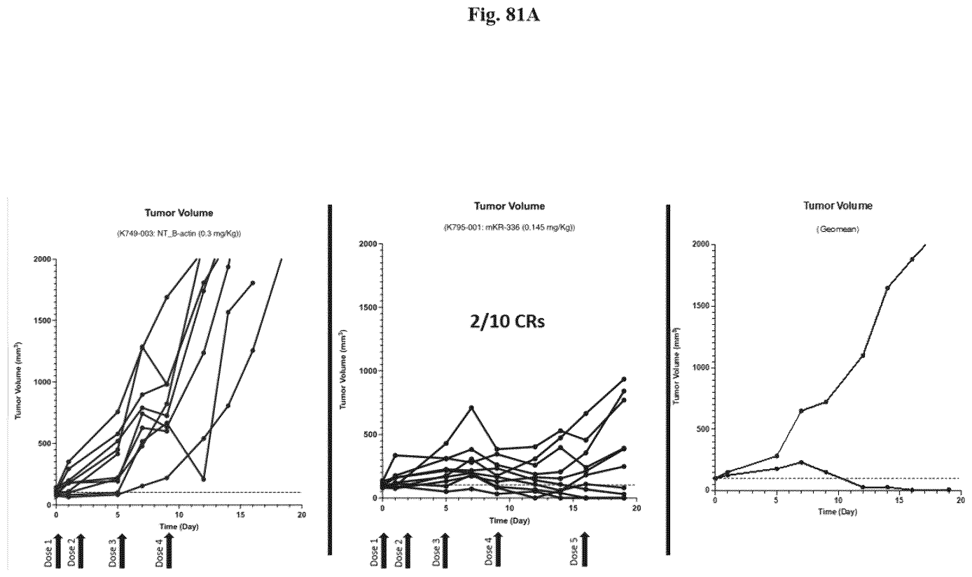

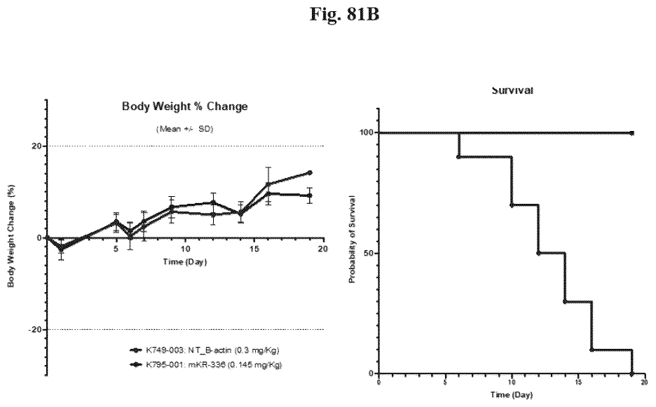

FIGS. 80A-C illustrate efficacy of KR-336 when administered systemically. The masking of mKR-336 (Pro072 or Pro073) was tested for tolerability and anti-tumor efficacy efficiency vs unmasked control (NS-336: Pro070 or Pro071) in the MC38.K C57BL/6 model. Peripheral mRNA-LNPs were employed and administered intravenously (IV) for systemic administration or intratumorally (IT) for comparison with direct tumor application. FIG. 80A illustrates mKR-336 was tolerated when administered IV, while NS-336 was not. FIG. 80B illustrates mKR-336 providing TGI benefit. FIG. 80C illustrates overall survival benefit (tumor burden limit at 1400 mm³). FIG. 81A and FIG. 81B illustrate efficacy of systemic administration of mKR-336 in MC38 tumor bearing C57BL/6 mice for determining if masking and half-life extension improved the therapeutic window and test for high tolerability of peripheral version with 4 LNP based mRNA payloads after repeated dosing. Comparison of mKR-336 at 0.145 mg/kg to NT B-actin Control was performed at 0.3 mg/Kg administered via tail vein injection. FIG. 81A illustrates mKR-336 systemic treatment resulted in tumor growth inhibition and completed responses, while being well tolerated. FIG. 81B illustrates mKR-336 with peripheral LNP V4 causing no body weight loss and providing a wide therapeutic window that allowed prolonged dose schedule. mKR-336 provided an overall survival benefit. As shown in FIG. 80 and FIG. 81, UTR-155 increased tumor/liver delivery ratio. Addition of FC increased the half-life of the mRNA transcribed protein. Addition of “Mask” decreased the peripheral activation and immune response. Peripheral Version 4 avoided immunogenicity caused by LNP systemic delivery.

### Example 11. Non-Human Primate (NHP) Assessment

Example 11 was to evaluate the pharmacodynamics of lipid nanoparticle (LNP) carrying human EPO-mRNA, KR-335, or KR-336 cargoes after intravenous (IV) or subcutaneous (SC) administration in cynomolgus monkeys. Study design involving three animals (Macaca fasciculari; Cynomolgus; males only; between ages of 4-6 years) per group and three groups is illustrated in Table 14. Pro061 (human IL-2), Pro080 (human IL-12), Pro081 (human IL-12), Pro099 (human IL-15), and Pro084 (human interferon α) were used in this NHP study. Dose selection was 0.1 mg/kg, which was approximately 500 mg mRNA LNP, enabling comparisons between all three groups for adverse event (AE) as compared to AE triggered in the animals by administration of human erythropoietin (EPO) mRNA. mKR-335 showed efficacy in mice at 0.02-20 mg, IT: Scaling >25× higher dose chosen for SC dose (500 mg) mKR-336 showed efficacy in mice at 0.05 mg/kg, IV,

TABLE 14
NHP study design

| | | | Dose | | Dose | |
|---|---|---|---|---|---|---|
| Group | No. of | Test | Level | Conc. | Volume | Dose Route/ |
| No. | Animals | Material | (mg/kg) | (mg/mL) | (mL/kg) | Regimen |
| 1 | 3 | hEPO-mRNA | 0.1 | 0.02 | 5 | Single IV (~60-minutes) |
| | | LNP Control | | | | weekly infusion for 4 |
| | | | | | | weeks |
| 2 | 3 | K637-mRNA | Total: | 0.143 | Total: | Single SC injection |
| | | KR-335 | 0.5 mg | | 3.5 ml | weekly for 4 weeks |
| 3 | 3 | K636-mRNA | 0.1 | 0.02 | 5 | Single IV (~60-minutes) |
| | | KR-336 | | | | Infusion |
| | | | | | | weekly for 4 weeks |

For intravenous (IV) dosing, Groups 1 and 3 animals received intravenous IV infusion of test article weekly for 4 weeks. The infusion was at 5 mL/kg/hour using an SAI 3D Programmable Syringe Pump (SAI 3D™) and a Male Luer Lock Adapter (Baxter 2C6227) extension set. Dosing was followed by 0.2 mL of vehicle to flush the dose from the IV catheter. Dose rate was at 5 ml/kg. The total infusion time was around one hour. Doses were administered through the saphenous or cephalic vein with an IV catheter. For subcutaneous (SC) dosing, Group 2 animals received SC injection of test article weekly for 4 weeks. The injection volume was 3.5 mL (0.143 mg/kg; total 500 ug). The research status of the animals was naïve, and their body weights were between four to seven kg. Table 15 illustrates the dosing sets

TABLE 15
Dosing sets

| Dosing Set | Group No. | Dosing order |
|---|---|---|
| A | 1 | First |
| | 2 | |
| | 3 | |
| B | 1 | Second (24 hours after |
| | 1 | set A first dosing) |
| | 2 | |
| | 2 | |
| | 3 | |
| | 3 | |

Blood sample was collection from an appropriate peripheral vein (not the vein used for dosing). according to the time points listed in Table 16 and collected into the tubes as indicated in Table 17.

TABLE 16
study activity schedule

| | Animal | Pre-Dose | 0 hr | 2 hrs | 6 hrs | 24 hrs | 48 hrs | 48 hrs |
|---|---|---|---|---|---|---|---|---|
| Activity | # | (D −1) | (D 0) | (D 0) | (D 0) | (D 1) | (D 2) | (D 2) |
| Dosing | 9 | — | X | — | — | — | — | — |
| Body | 9 | X | — | — | — | X | X | — |
| weight | | | | | | | | |
| Body | 9 | X | — | — | X | X | — | — |
| temperature | | | | | | | | |
| Blood | 9 | X | — | X | X | X | X | — |
| collection | | | | | | | | |
| Blood | NA | PK | NA | PK | PK | PK | PK | — |
| analysis | | Hematology | | — | — | Hematology | | |
| | | Coagulation | | — | — | Coagulation | | |
| | | Clin. | | — | — | Clin. | | |
| | | Chem. | | — | — | Chem. | | |
| | | Cytokine | | — | Complement | Cytokin [symbol] | | |
| | | [symbol] | | | | [symbol] | | |
| 5 mL | — | — | — | — | — | — | — | X |
| saline | | | | | | | | |
| SC | | | | | | | | |
| replace [symbol] | | | | | | | | |
| Total | NA | 6 | NA | 2 | 2 | 6 | 2 | NA |
| volume | | | | | | | | |
| of blood | | | | | | | | |
| collection | | | | | | | | |
| (mL) | | | | | | | | |
| | Animal | Pre-Dose | 0 hr | 2 hrs | 6 hrs | 24 hrs | 48 hrs | 48 hrs |
| Activity | # | (d 6) | (D 7) | (D 7) | (D 7) | (D 8) | (D 9) | (D 9) |
| Dosing | 9 | — | X | — | — | — | — | — |
| Body | 9 | X | — | — | — | X | X | — |
| weight | | | | | | | | |
| Body | 9 | X | — | — | X | X | — | — |
| temperature | | | | | | | | |
| Blood | 9 | X | — | X | X | X | X | — |
| collection | | | | | | | | |
| Blood | NA | PK | NA | PK | PK | PK | PK | — |
| analysis | | Hematology | | — | — | Hematology | | |
| | | Coagulation | | — | — | Coagulation | | |
| | | Clin. | | — | — | Clin. | | |
| | | Chem. | | — | — | Chem. | | |
| | | Cytokine | | — | Complement | Cytokine | | |
| | | Complement | | | | Complement | | |
| 5 mL | — | — | — | — | — | — | — | X |
| saline | | | | | | | | |
| SC | | | | | | | | |
| replacement | | | | | | | | |
| Total | NA | 6 | NA | 2 | 2 | 6 | 2 | NA |
| volume | | | | | | | | |
| of blood | | | | | | | | |
| collection | | | | | | | | |
| (mL) | | | | | | | | |

| | Animal | Pre-Dose | 0 hr | 2 hrs | 6 hrs | 24 hrs | 48 hrs | 48 hrs |
|---|---|---|---|---|---|---|---|---|
| Activity | # | (D 13) | (D 14) | (D 14) | (D 14) | (D 15) | (D 16) | (D 16) |
| Dosing | 9 | — | X | — | — | — | — | — |
| Body | 9 | X | — | — | — | X | X | — |
| weights | | | | | | | | |
| Body | 9 | X | — | — | X | X | — | — |
| temperature | | | | | | | | |
| Blood | 9 | X | — | X | X | X | X | — |
| collection | | | | | | | | |
| Blood | NA | PK | NA | PK | PK | PK | PK | — |
| analysis | | Hematology | | — | Complement | Hematology | | |
| | | Coagulation | | — | | Coagulation | | |
| | | Clin. | | — | | Clin. | | |
| | | Chem. | | — | | Chem. | | |
| | | Cytokine | | | | Cytokine | | |
| 5 mL | — | — | — | — | — | — | X | X |
| saline | | | | | | | | |
| SC | | | | | | | | |
| replacement | | | | | | | | |
| Total | NA | 6 | NA | 2 | 2 | 6 | 2 | NA |
| volume | | | | | | | | |
| of blood | | | | | | | | |
| collection | | | | | | | | |
| (mL) | | | | | | | | |
| | | | 0 hr | 2 hrs | | | | |
| | Animal | Pre-Dose | (D 21) | (d 21) | 6 hrs | 24 hrs | 48 hrs | 48 hrs |
| Activity | # | (D 20) | (D 21) | (D 21) | (D 21) | (D 22) | (D 23) | (D 23) |
| Dosing | 9 | — | X | — | — | — | — | — |
| Body | 9 | X | — | — | — | X | X | — |
| weight | | | | | | | | |
| Body | 9 | X | — | — | X | X | — | — |
| temperature | | | | | | | | |
| Blood | 9 | X | — | X | X | X | X | — |
| collection | | | | | | | | |
| Blood | NA | PK | NA | PK | PK | PK | PK | — |
| analysis | | Hematology | | — | Complement | Hematology | | |
| | | Coagulation | | — | | Coagulation | | |
| | | Clin. | | — | | Clin. | | |
| | | Chem. | | — | | Chem. | | |
| | | Cytokine | | | | Cytokine | | |
| | | Complement | | | | Complement | | |
| 5 mL | — | — | — | — | — | — | X | X |
| saline | | | | | | | | |
| SC | | | | | | | | |
| replace [symbol] | | | | | | | | |
| Total | NA | 6 | NA | 2 | 2 | 6 | 2 | NA |
| volume | | | | | | | | |
| of blood | | | | | | | | |
| collection | | | | | | | | |
| (mL) | | | | | | | | |

| Activity | Animal # | Day 28 |
|---|---|---|
| Dosing | 9 | — |
| Body | 9 | X |
| weight | | |
| Body | 9 | — |
| temperature | | |
| Blood | 9 | X |
| collection | | |
| Blood | NA | PK |
| analysis | | Hematology |
| | | Coagulation |
| | | Clin. |
| | | Chem. |
| | | Cytokine |
| | | Complem [symbol] |
| Total | NA | 6 |
| volume | | |
| of blood | | |
| collection | | |
| (mL) | | |

X = event for all animals and set;
# = number of animals;
D = Day;
SC = subcutaneous [symbol]
indicates data missing or illegible when filed

TABLE 17
Blood sample collection

| | | | | | Serum for | |
|---|---|---|---|---|---|---|
| | | | | Serum for | Sponsor | Serum for |
| | | | Serum | PK/PD | Cytokine | Complement |
| Time Point | Hematology | Coagulation | Chemistry | Analysis | Analysis | Analysis |
| Additive | K₂EDTA | Sodium | None (SST) | | | |
| | | Citrate | | | | |
| ~Volume of | 0.25 mL | 1.3 mL | 4.45 mL | | | |
| Whole Blood | | | | | | |

Hematology, clinical chemistry, and coagulation were performed. After collection, blood samples (4.45 ml) were processed for serum (2000×g for 10 minutes at 4° C.). Whole blood hematology samples (0.25 ml) were stored at 2-8° C. Blood samples for coagulation (1.3 ml) were processed to citrated plasma (1500 rpm for 15 minutes at 4° C.) and frozen until shipped (400 μL). For the analysis, the body weights and markers associated with adverse event (AE) of the animals were monitored during the study. Markers included measurement of aspartate aminotransferase (AST), alanine transaminase (ALT), alkaline phosphatase (ALP), and gamma-glutamyl transpeptidase (GGT); and white blood cell count.

FIG. 82A illustrates body weight change of the three group of animals with the animals dosed on day 0, day 7, day 14, or day 21 of the study. On day 16, significant body weight difference was observed in hEPO treated group versus the KR-335 treated group (p=0.04; 2-way ANOVA). No significant body weight change was observed between groups on all other timepoints. FIG. 82B illustrates body weight change of the individual animals with the animals dosed on day 0, day 7, day 14, or day 21 of the study.

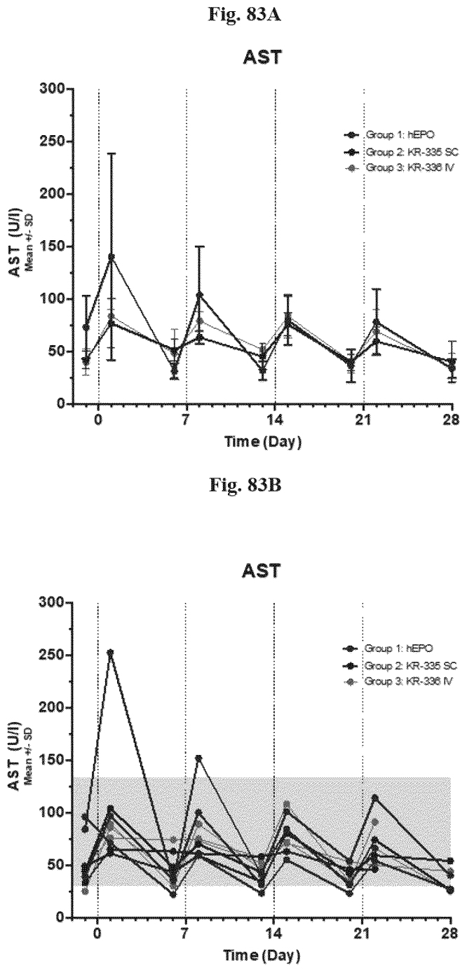

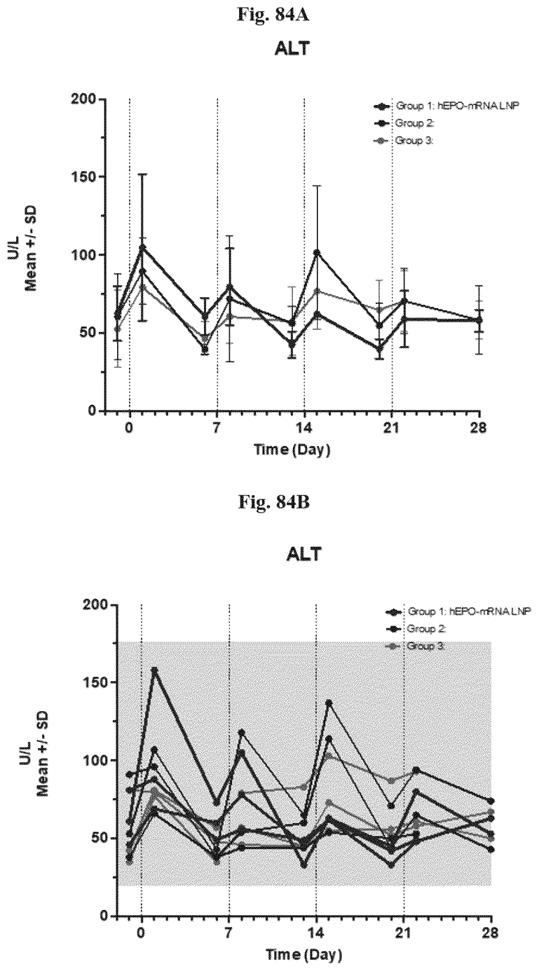

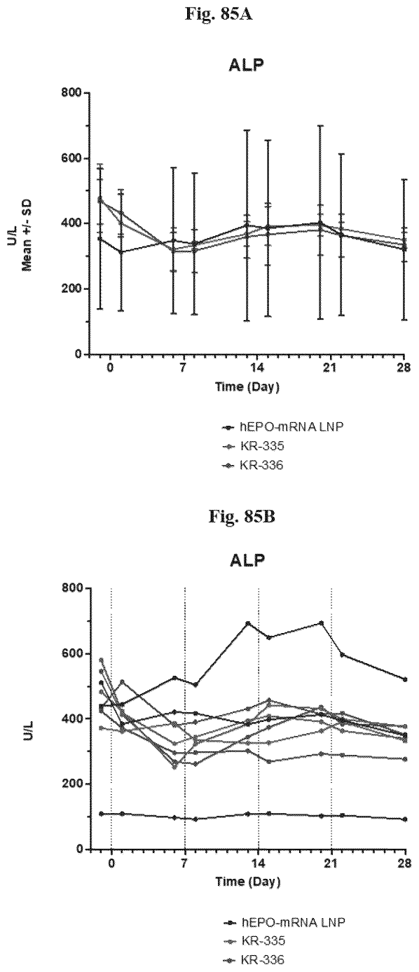

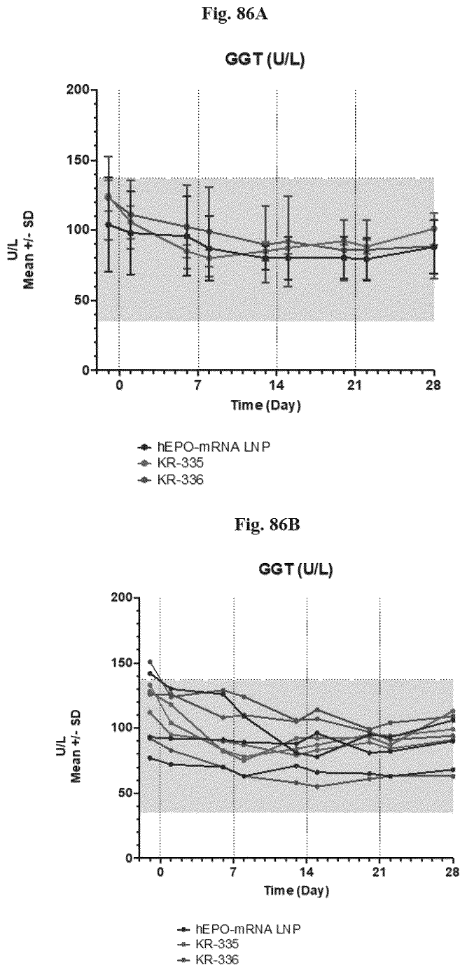

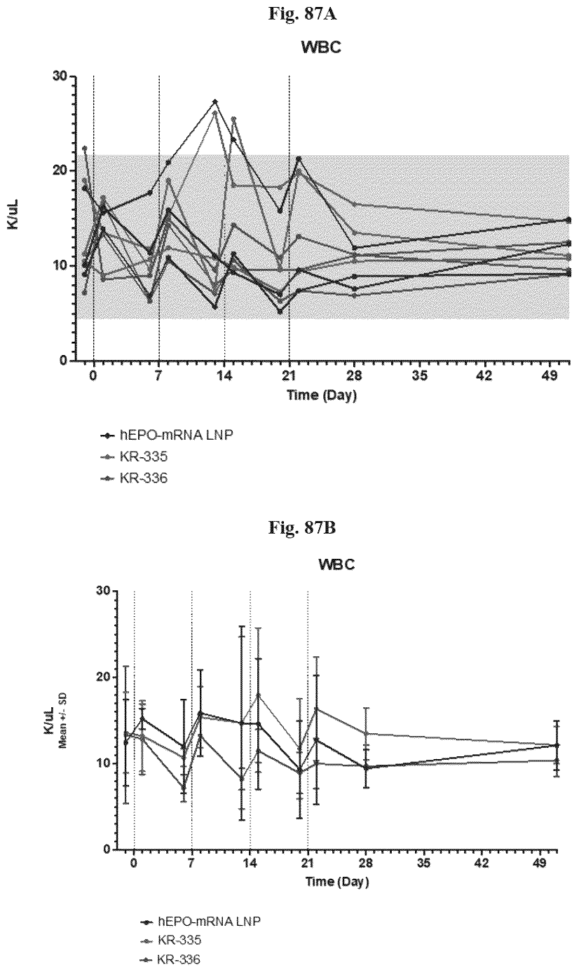

FIG. 83A illustrates aspartate aminotransferase (AST) measurement of the three group of animals during the study. No significant AST differences were observed based on all 2-way ANOVA group comparison. FIG. 83B illustrates aspartate aminotransferase (AST) measurement of the individual animals during the study. FIG. 84A illustrates alanine transaminase (ALT) measurement of the three group of animals during the study. No significant ALT differences were observed based on all 2-way ANOVA group comparison. FIG. 84B illustrates alanine transaminase (ALT) measurement of the individual animals during the study. FIG. 85A illustrates alkaline phosphatase (ALP) measurement of the three group of animals during the study. No significant ALP differences were observed based on all 2-way ANOVA group comparison. FIG. 85B illustrates alkaline phosphatase (ALP) measurement of the individual animals during the study. FIG. 86A illustrates gamma-glutamyl transpeptidase (GGT) measurement of the three group of animals during the study. No significant GGT differences were observed based on all 2-way ANOVA group comparison. FIG. 86B illustrates gamma-glutamyl transpeptidase (GGT) measurement of the individual animals during the study. FIG. 87A illustrates white blood cell (WBC) count of the three group of animals during the study. No significant WBC count differences were observed based on all 2-way ANOVA group comparison. FIG. 87B illustrates white blood cell (WBC) measurement of the individual animals during the study. Treatment was overall tolerated. No changes in behavior were observed. No major changes were observed in body weight (BW), temperature, liver enzymes, or other blood chemistry that would indicate severe AEs.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

IL-12 amino acid sequence table

| SEQ ID | | |
|---|---|---|
| NO | Amino acid sequence | Notes |
| SEQ ID | METDTLLLWVLLLWVPGSTG | IL-12 |
| NO: | MWELEKDVYVVEVDWTPDAPGETVNLTCDTPEEDDITWTSDQRHGVIGSGKTLTIT | construct: |
| 301 | VKEFLDAGQYTCHKGGETLSHSHLLLHKKENGIWSTEILKNEKNKTFLKCEAPNYSG | single chain |
| | RFTCSWLVQRNMDLKFNIKSSSSSPDSRAVTCGMASLSAEKVTLDQRDYEKYSVSC | option; |
| | QEDVTCPTAEETLPIELALEARQQNKYENYSTSFFIRDIIKPDPPKNLQMKPLKNSQVE | Signal |
| | VSWEYPDSWSTPHSYFSLKFFVRIQRKKEKMKETEEGCNQKGAFLVEKTSTEVQCK | peptide- |
| | GGNVCVQAQDRYYNSSCSKWACVPCRVRS | p40- |
| | GGGGSGGGGSGGGGS | linker- |
| | RVIPVSGPARCLSQSRNLLKTTDDMVKTAREKLKHYSCTAEDIDHEDITRDQTSTLKT | p35- |
| | CLPLELHKNESCLATRETSSTTRGSCLPPQKTSLMMTLCLGSIYEDLKMYQTEFQAIN | cleavable |
| | AALQNHNHQQIILDKGMLVAIDELMQSINHNGETLRQKPPVGEADPYRVKMKLCIL | linker- |
| | LHAFSTRVVTINRVMGYLSSA | Masking- |
| | HPVGLLARVPLSLYSG HPVGLLARVPLSLYSGLSGRSDNHGGGSGGGS | linker-Fc |
| | QLGASGPGDGCCVEKTSFPEGASGSPLGPRNLSCYRVSKTDYECSWQYDGPEDNVS | LALAPG |
| | HVLWCCFVPPNHTHTGQERCRYFSSGPDRTVQFWEQDGIPVLSKVNFWVESRLGNR | |
| | TMKSQKISQYLYNWTKTTPPLGHIKVSQSHRQLRMDWNVSEEAGAEVQFRRRMPTT | |
| | NWTLGDCGPQVNSGSGVLGDIRGSMSESCLCPSENMAQEIQIRRRRRLSSGAPGGPW | |
| | SDWSMPVCVPPEVLPQA | |
| | GGGGSGGGGSGGGGS | |
| | GPTIKPCPPCKCPAPNAAGGPSVFIFPPKIKDVLM | |
| | ISLSPIVTCVVVDVSEDDPDVQISWFVNNVEVHTA | |
| | QTQTHREDYNSTLRVVSALPIQHQDWMSGKEEKCK | |
| | VNNKDLGAPIERTISKPKGSVRAPQVYVLPPPEEE | |
| | MTKKQVTLTCMVTDFMPEDIYVEWTNNGKTELNYK | |
| | NTEPVLDSDGSYFMYSKLRVEKKNWVERNSYSCSV | |
| | VHEGLHNHHTTKSFSRTPG | |
| SEQ ID | METDTLLLWVLLLWVPGSTG | IL-12 |
| NO: | GPTIKPCPPCKCPAPNAAGGPSVFIFPPKIKDVLM | construct: |
| 302 | ISLSPIVTCVVVDVSEDDPDVQISWFVNNVEVHTA | single chain |
| | QTQTHREDYNSTLRVVSALPIQHQDWMSGKEFKCK | option; |
| | VNNKDLGAPIERTISKPKGSVRAPQVYVLPPPEEE | Signal |
| | MTKKQVTLTCMVTDEMPEDIYVEWTNNGKTELNYK | peptide- |
| | NTEPVLDSDGSYFMYSKLRVEKKNWVERNSYSCSV | Fc |
| | VHEGLHNHHTTKSFSRTPG | LALAPG- |
| | GGGGSGGGGSGGGGS | linker- |
| | QLGASGPGDGCCVEKTSFPEGASGSPLGPRNLSCYRVSKTDYECSWQYDGPEDNVS | Masking- |
| | HVLWCCFVPPNHTHTGQERCRYFSSGPDRTVQFWEQDGIPVLSKVNFWVESRLGNR | cleavable |
| | TMKSQKISQYLYNWTKTTPPLGHIKVSQSHRQLRMDWNVSEEAGAEVQFRRRMPTT | linker- |
| | NWTLGDCGPQVNSGSGVLGDIRGSMSESCLCPSENMAQEIQIRRRRRLSSGAPGGPW | p40- |
| | SDWSMPVCVPPEVLPQA | p35 |
| | HPVGLLARVPLSLYSGHPVGLLARVPLSLYSGLSGRSDNHGGGSGGGS | |
| | MWELEKDVYVVEVDWTPDAPGETVNLTCDTPEEDDITWTSDQRHGVIGSGKTLTIT | |
| | VKEFLDAGQYTCHKGGETLSHSHLLLHKKENGIWSTEILKNFKNKTFLKCEAPNYSG | |
| | RFTCSWLVQRNMDLKFNIKSSSSSPDSRAVTCGMASLSAEKVTLDQRDYEKYSVSC | |
| | QEDVTCPTAEETLPIELALEARQQNKYENYSTSFFIRDIIKPDPPKNLQMKPLKNSQVE | |
| | VSWEYPDSWSTPHSYFSLKFFVRIQRKKEKMKETEEGCNQKGAFLVEKTSTEVQCK | |
| | GGNVCVQAQDRYYNSSCSKWACVPCRVRS | |
| | GGGGSGGGGSGGGGS | |
| | RVIPVSGPARCLSQSRNLLKTTDDMVKTAREKLKHYSCTAEDIDHEDITRDQTSTLKT | |
| | CLPLELHKNESCLATRETSSTTRGSCLPPQKTSLMMTLCLGSIYEDLKMYQTEFQAIN | |
| | AALQNHNHQQIILDKGMLVAIDELMQSLNHNGETLRQKPPVGEADPYRVKMKLCIL | |
| | LHAFSTRVVTINRVMGYLSSA | |
| SEQ ID | METDTLLLWVLLLWVPGSTG | IL-12 |
| NO: | QLGASGPGDGCCVEKTSFPEGASGSPLGPRNLSCYRVSKTDYECSWQYDGPEDNVS | construct: |
| 303 | HVLWCCFVPPNHTHTGQERCRYFSSGPDRTVQFWEQDGIPVLSKVNEWVESRLGNR | single chain |
| | TMKSQKISQYLYNWTKTTPPLGHIKVSQSHRQLRMDWNVSEEAGAEVQFRRRMPTT | option; |
| | NWTLGDCGPQVNSGSGVLGDIRGSMSESCLCPSENMAQEIQIRRRRRLSSGAPGGPW | Signal- |
| | SDWSMPVCVPPEVLPQA | peptide- |
| | HPVGLLARVPLSLYSGHPVGLLARVPLSLYSGLSGRSDNHGGGSGGGS | Masking- |
| | MWELEKDVYVVEVDWTPDAPGETVNLTCDTPEEDDITWTSDQRHGVIGSGKTLTIT | cleavable |
| | VKEFLDAGQYTCHKGGETLSHSHLLLHKKENGIWSTEILKNEKNKTFLKCEAPNYSG | linker- |
| | RFTCSWLVQRNMDLKFNIKSSSSSPDSRAVTCGMASLSAEKVILDQRDYEKYSVSC | p40- |
| | QEDVTCPTAEETLPIELALEARQQNKYENYSTSFFIRDIIKPDPPKNLQMKPLKNSQVE | p35- |
| | VSWEYPDSWSTPHSYESLKFFVRIQRKKEKMKETEEGCNQKGAFLVEKTSTEVQCK | cleavable |
| | GGNVCVQAQDRYYNSSCSKWACVPCRVRS | linker- |
| | GGGGSGGGGSGGGGS | Fc |
| | RVIPVSGPARCLSQSRNLLKTTDDMVKTAREKLKHYSCTAEDIDHEDITRDQTSTLKT | LALAPG |
| | CLPLELHKNESCLATRETSSTTRGSCLPPQKTSLMMTLCLGSIYEDLKMYQTEFQAIN | |
| | AALQNHNHQQIILDKGMLVAIDELMQSLNHNGETLRQKPPVGEADPYRVKMKLCIL | |
| | LHAFSTRVVTINRVMGYLSSA | |
| | HPVGLLARVPLSLYSGHPVGLLARVPLSLYSGLSGRSDNHGGGSGGGS | |
| | GPTIKPCPPCKCPAPNAAGGPSVFIFPPKIKDVLM | |
| | ISLSPIVTCVVVDVSEDDPDVQISWFVNNVEVHTA | |
| | QTQTHREDYNSTLRVVSALPIQHQDWMSGKEEKCK | |
| | VNNKDLGAPIERTISKPKGSVRAPQVYVLPPPEEE | |
| | MTKKQVTLTCMVTDEMPEDIYVEWTNNGKTELNYK | |
| | NTEPVLDSDGSYFMYSKLRVEKKNWVERNSYSCSV | |
| | VHEGLHNHHTTKSFSRTPG | |
| SEQ ID | METDTLLLWVLLLWVPGSTG | Control 1: |
| NO: | MWELEKDVYVVEVDWTPDAPGETVNLTCDTPEEDDITWTSDQRHGVIGSGKTLTIT | Signal |
| 331 | VKEFLDAGQYTCHKGGETLSHSHLLLHKKENGIWSTEILKNEKNKTFLKCEAPNYSG | Peptide- |
| | RFTCSWLVQRNMDLKFNIKSSSSSPDSRAVTCGMASLSAEKVTLDQRDYEKYSVSC | p40- |
| | QEDVTCPTAEETLPIELALEARQQNKYENYSTSFFIRDIIKPDPPKNLQMKPLKNSQVE | linker- |
| | VSWEYPDSWSTPHSYFSLKFFVRIQRKKEKMKETEEGCNQKGAFLVEKTSTEVQCK | p35- |
| | GGNVCVQAQDRYYNSSCSKWACVPCRVRS | non |
| | GGGGSGGGGSGGGGS | cleavable |
| | RVIPVSGPARCLSQSRNLLKTTDDMVKTAREKLKHYSCTAEDIDHEDITRDQTSTLKT | linker- |
| | CLPLELHKNESCLATRETSSTTRGSCLPPQKTSLMMTLCLGSIYEDLKMYQTEFQAIN | Masking- |
| | AALQNHNHQQIILDKGMLVAIDELMQSLNHNGETLRQKPPVGEADPYRVKMKLCIL | linker- |
| | LHAFSTRVVTINRVMGYLSSA | Fc |
| | GGGGSGGGGSGGGGS | LALAP |
| | QLGASGPGDGCCVEKTSFPEGASGSPLGPRNLSCYRVSKTDYECSWQYDGPEDNVS | [G/A] |
| | HVLWCCFVPPNHTHTGQERCRYFSSGPDRTVQFWEQDGIPVLSKVNFWVESRLGNR | |
| | TMKSQKISQYLYNWTKTTPPLGHIKVSQSHRQLRMDWNVSEEAGAEVQFRRRMPTT | |
| | NWTLGDCGPQVNSGSGVLGDIRGSMSESCLCPSENMAQEIQIRRRRRLSSGAPGGPW | |
| | SDWSMPVCVPPEVLPQA | |
| | GGGGSGGGGSGGGGS | |
| | GPTIKPCPPCKCPAPNAAGGPSVFIFPPKIKDVLM | |
| | ISLSPIVTCVVVDVSEDDPDVQISWFVNNVEVHTA | |
| | QTQTHREDYNSTLRVVSALPIQHQDWMSGKEFKCK | |
| | VNNKDLGAPIERTISKPKGSVRAPQVYVLPPPEEE | |
| | MTKKQVTLTCMVTDEMPEDIYVEWTNNGKTELNYK | |
| | NTEPVLDSDGSYFMYSKLRVEKKNWVERNSYSCSV | |
| | VHEGLHNHHTTKSFSRTPG | |
| SEQ ID | METDTLLLWVLLLWVPGSTG | Control 2a: |
| NO: | MWELEKDVYVVEVDWTPDAPGETVNLTCDTPEEDDITWTSDQRHGVIGSGKTLTIT | Signal |
| 332 | VKEFLDAGQYTCHKGGETLSHSHLLLHKKENGIWSTEILKNEKNKTFLKCEAPNYSG | Peptide- |
| | RFTCSWLVQRNMDLKFNIKSSSSSPDSRAVTCGMASLSAEKVTLDQRDYEKYSVSC | p40- |
| | QEDVTCPTAEETLPIELALEARQQNKYENYSTSFFIRDIIKPDPPKNLQMKPLKNSQVE | linker- |
| | VSWEYPDSWSTPHSYFSLKFFVRIQRKKEKMKETEEGCNQKGAFLVEKTSTEVQCK | p35 |
| | GGNVCVQAQDRYYNSSCSKWACVPCRVRS | |
| | GGGGSGGGGSGGGGS | |
| | RVIPVSGPARCLSQSRNLLKTTDDMVKTAREKLKHYSCTAEDIDHEDITRDQTSTLKT | |
| | CLPLELHKNESCLATRETSSTTRGSCLPPQKTSLMMTLCLGSIYEDLKMYQTEFQAIN | |
| | AALQNHNHQQIILDKGMLVAIDELMQSINHNGETLRQKPPVGEADPYRVKMKLCIL | |
| | LHAFSTRVVTINRVMGYLSSA | |
| SEQ ID | MGAMAPRTLLLLLAAALAPTQTRAGPGS | Control 2b: |
| NO: | MWELEKDVYVVEVDWTPDAPGETVNLTCDTPEEDDITWTSDQRHGVIGSGKTLTIT | Siqnal |
| 333 | VKEFLDAGQYTCHKGGETLSHSHLLLHKKENGIWSTEILKNFKNKTFLKCEAPNYSG | Peptide- |
| | RFTCSWLVQRNMDLKENIKSSSSSPDSRAVTCGMASLSAEKVTLDQRDYEKYSVSC | p40- |
| | QEDVTCPTAEETLPIELALEARQQNKYENYSTSFFIRDIIKPDPPKNLQMKPLKNSQVE | linker- |
| | VSWEYPDSWSTPHSYFSLKFFVRIQRKKEKMKETEEGCNQKGAFLVEKTSTEVQCK | p35 |
| | GGNVCVQAQDRYYNSSCSKWACVPCRVRS | |
| | VPGVGVPGVG | |
| | RVIPVSGPARCLSQSRNLLKTTDDMVKTAREKLKHYSCTAEDIDHEDITRDQTSTLKT | |
| | CLPLELHKNESCLATRETSSTTRGSCLPPQKTSLMMTLCLGSIYEDLKMYQTEFQAIN | |
| | AALQNHNHQQIILDKGMLVAIDELMQSLNHNGETLRQKPPVGEADPYRVKMKLCIL | |
| | LHAFSTRVVTINRVMGYLSSA | |
| SEQ ID | METDTLLLWVLLLWVPGSTG | Control |
| NO: | MWELEKDVYVVEVDWTPDAPGETVNLTCDTPEEDDITWTSDQRHGVIGSGKTLTIT | 3: |
| 334 | VKEFLDAGQYTCHKGGETLSHSHLLLHKKENGIWSTEILKNEKNKTFLKCEAPNYSG | Signal |
| | RFTCSWLVQRNMDLKFNIKSSSSSPDSRAVTCGMASLSAEKVTLDQRDYEKYSVSC | Peptide- |
| | QEDVTCPTAEETLPIELALEARQQNKYENYSTSFFIRDIIKPDPPKNLQMKPLKNSQVE | p40- |
| | VSWEYPDSWSTPHSYESLKFFVRIQRKKEKMKETEEGCNQKGAFLVEKTSTEVQCK | linker- |
| | GGNVCVQAQDRYYNSSCSKWACVPCRVRS | p35- |
| | GGGGGGGGSGGGGS | cleavable |
| | RVIPVSGPARCLSQSRNLLKTTDDMVKTAREKLKHYSCTAEDIDHEDITRDQTSTLKT | linker- |
| | CLPLELHKNESCLATRETSSTTRGSCLPPQKTSLMMTLCLGSIYEDLKMYQTEFQAIN | Masking |
| | AALQNHNHQQIILDKGMLVAIDELMQSINHNGETLRQKPPVGEADPYRVKMKLCIL | |
| | LHAFSTRVVTINRVMGYLSSA | |
| | HPVGLLARVPLSLYSGHPVGLLARVPLSLYSGLSGRSDNHGGGSGGGS | |
| | QLGASGPGDGCCVEKTSFPEGASGSPLGPRNLSCYRVSKTDYECSWQYDGPEDNVS | |
| | HVLWCCFVPPNHTHTGQERCRYFSSGPDRTVQFWEQDGIPVLSKVNFWVESRLGNR | |
| | TMKSQKISQYLYNWTKTTPPLGHIKVSQSHRQLRMDWNVSEEAGAEVQFRRRMPTT | |
| | NWTLGDCGPQVNSGSGVLGDIRGSMSESCLCPSENMAQEIQIRRRRRLSSGAPGGPW | |
| | SDWSMPVCVPPEVLPQA | |
| SEQ ID | METDTLLLWVLLLWVPGSTG | Control |
| NO: | MWELEKDVYVVEVDWTPDAPGETVNLTCDTPEEDDITWTSDQRHGVIGSGKTLTIT | 4: |
| 335 | VKEFLDAGQYTCHKGGETLSHSHLLLHKKENGIWSTEILKNEKNKTFLKCEAPNYSG | Signal |
| | RFTCSWLVQRNMDLKENIKSSSSSPDSRAVTCGMASLSAEKVTLDQRDYEKYSVSC | peptide- |
| | QEDVTCPTAEETLPIELALEARQQNKYENYSTSFFIRDIIKPDPPKNLQMKPLKNSQVE | p40- |
| | VSWEYPDSWSTPHSYFSLKFFVRIQRKKEKMKETEEGCNQKGAFLVEKTSTEVQCK | linker- |
| | GGNVCVQAQDRYYNSSCSKWACVPCRVRS | p35- |
| | GGGGSGGGGSGGGGS | linker- |
| | RVIPVSGPARCLSQSRNLLKTTDDMVKTAREKLKHYSCTAEDIDHEDITRDQTSTLKT | Fc |
| | CLPLELHKNESCLATRETSSTTRGSCLPPQKTSLMMTLCLGSIYEDLKMYQTEFQAIN | LALAPG |
| | AALQNHNHQQIILDKGMLVAIDELMQSLNHNGETLRQKPPVGEADPYRVKMKLCIL | |
| | LHAFSTRVVTINRVMGYLSSA | |
| | GGGGSGGGGSGGGGS | |
| | GPTIKPCPPCKCPAPNAAGGPSVFIFPPKIKDVLM | |
| | ISLSPIVTCVVVDVSEDDPDVQISWFVNNVEVHTA | |
| | QTQTHREDYNSTLRVVSALPIQHQDWMSGKEFKCK | |
| | VNNKDLGAPIERTISKPKGSVRAPQVYVLPPPEEE | |
| | MTKKQVTLTCMVTDEMPEDIYVEWINNGKTELNYK | |
| | NTEPVLDSDGSYFMYSKLRVEKKNWVERNSYSCSV | |
| | VHEGLHNHHTTKSFSRTPG | |
| SEQ ID | METDTLLLWVLLLWVPGSTG | Signal |
| NO: | RVIPVSGPARCLSQSRNLLKTTDDMVKTAREKLKHYSCTAEDIDHEDITRDQTSTLKTCLPLELH | peptide- |
| 331 | KNESCLATRETSSTTRGSCLPPQKTSLMMTLCLGSIYEDLKMYQTEFQAINAALQNHNHQQIILD | p35- |
| | KGMLVAIDELMQSLNHNGETLRQKPPVGEADPYRVKMKLCILLHAFSTRVVTINRVMGYLSSA | cleavable |
| | HPVGLLARVPLSLYSGHPVGLLARVPLSLYSGLSGRSDNHGGGSGGGS | linker- |
| | PRGPTIKPCPPCKCPAPNAAGGPSVFIFPPKIKDVLMISLSPIVTCVVVDVSEDDPDVQISWFVNNV | Fc- |
| | EVHTA | knob- |
| | EEE | EW-LALAPG; |
| | MTEKQVTLTCMVTDEMPEDIYVEWTNNGKTELNYKNTEPVLDSDGSYFMYSWLRVEKKNWVERNSYS | mIL- |
| | CSV | 12- |
| | VHEGLHNHHTTKSFSRTPG | 1st chain: |
| | | (492 aa) |
| | | pro070 |
| SEQ ID | METDTLLLWVLLLWVPGSTG | Signal |
| NO: | MWELEKDVYVVEVDWTPDAPGETVNLTCDTPEEDDITWTSDQRHGVIGSGKTLTITVKEFLDAGQYT | peptide- |
| 312 | CHKGGETLSHSHLLLHKKENGIWSTEILKNFKNKTFLKCEAPNYSGRFTCSWLVQRNMDLKENIKSS | p40- |
| | SSSPDSRAVTCGMASLSAEKVTLDQRDYEKYSVSCQEDVTCPTAEETLPIELALEARQQNKYENYST | cleavable |
| | SFFIRDIIKPDPPKNLQMKPLKNSQVEVSWEYPDSWSTPHSYFSLKFFVRIQRKKEKMKETEEGCNQ | linker- |
| | KGAFLVEKTSTEVQCKGGNVCVQAQDRYYNSSCSKWACVPCRVRS | Fc- |
| | HPVGLLARVPLSLYSGHPVGLLARVPLSLYSGLSGRSDNHGGGSGGGS | hole- |
| | PRGPTIKPCPPCKCPAPNAAGGPSVFIFPPKIKDVLMISLSPIVTCVVVDVSEDDPDVQISWFVNNV | RVT- |
| | EVHTA | LALAPG; |
| | QTQTHREDYNSTLRVVSALPIQHQDWMSGKEFKCKVNNKDLGAPIERTISKPKGSVRAPRVYVLPPP | mIL- |
| | EEE | 12- |
| | MTKKQVTLTCMVTDEMPEDIYVEWTNNGKTELNYKNTEPVLVSDGSYTMYSKLRVEKKNWVERNSYS | 2nd |
| | CSVVHEGLHNHHTTKSFSRTPG | chain: |
| | | (612 aa) |
| | | pro071 |
| SEQ ID | METDTLLLWVLLLWVPGSTG | Signal |
| NO: | QLGASGPGDGCCVEKTSFPEGASGSPLGPRNLSCYRVSKTDYECSWQYDGPEDNVS | peptide- |
| 313 | HVLWCCFVPPNHTHTGQERCRYFSSGPDRTVQFWEQDGIPVLSKVNFWVESRLGNR | Mask |
| | TMKSQKISQYLYNWTKTTPPLGHIKVSQSHRQLRMDWNVSEEAGAEVQFRRRMPTT | (IL-12Rb1)-- |
| | NWTLGDCGPQVNSGSGVLGDIRGSMSESCLCPSENMAQEIQIRRRRRLSSGAPGGPW | cleavable |
| | SDWSMPVCVPPEVLPQA | linker-- |
| | HPVGLLARVPLSLYSGHPVGLLARVPLSLYSGLSGRSDNHGGGSGGGS | p35- |
| | RVIPVSGPARCLSQSRNLLKTTDDMVKTAREKLKHYSCTAEDIDHEDITRDQTSTLKTCLPLELH | cleavable |
| | KNESCLATRETSSTTRGSCLPPQKTSLMMTLCLGSIYEDLKMYQTEFQAINAALQNHNHQQIILD | linker- |
| | KGMLVAIDELMQSLNHNGETLRQKPPVGEADPYRVKMKLCILLHAFSTRVVTINRVMGYLSSA | Fc |
| | HPVGLLARVPLSLYSGHPVGLLARVPLSLYSGLSGRSDNHGGGSGGGS | (EW- |
| | PRGPTIKPCPPCKCPAPNAAGGPSVFIFPPKIKDVLMISLSPIVTCVVVDVSEDDPDVQISWFVNNV | LALAPG); |
| | EVHTA | mIL- |
| | QTQTHREDYNSTLRVVSALPIQHQDWMSGKEFKCKVNNKDLGAPIERTISKPKGSVRAPQVYVLPPP | 12- |
| | EEE | 1st |
| | MTEKQVTLTCMVTDEMPEDIYVEWTNNGKTELNYKNTEPVLDSDGSYFMYSWLRVEKKNWVERNSYS | chain- |
| | CSV | masked: |
| | VHEGLHNHHTTKSFSRTPG | (782 aa) |
| | | pro072 |
| SEQ ID | METDTLLLWVLLLWVPGSTG | Signal |
| NO: | NIDVCKLGTVTVQPAPVIPLGSAANISCSLNPKQGCSHYPSSNELILLKFVNDVLVENLHGKKVHDH | peptide- |
| 314 | TGHSSTFQVTNLSLGMTLFVCKLNCSNSQKKPPVPVCGVEISVGVAPEPPQNISCVQEGENGTVACS | Mask |
| | WNSGKVTYLKTNYTLQLSGPNNLTCQKQCFSDNRQNCNRLDLGINLSPDLAESRFIVRVTAINDLGN | (IL-12Rb2)- |
| | SSSLPHTFTFLDIVIPLPPWDIRINELNASGSRGTLQWEDEGQVVLNQLRYQPLNSTSWNMVNATNA | cleavable |
| | KGKYDLRDLRPFTEYEFQISSKLHLSGGSWSNWSESLRTRTPEE | linker-- |
| | HPVGLLARVPLSLYSGHPVGLLARVPLSLYSGLSGRSDNHGGGSGGGS | p40- |
| | MWELEKDVYVVEVDWTPDAPGETVNLTCDTPEEDDITWTSDQRHGVIGSGKTLTITVKEFLDAGQYT | cleavable |
| | CHKGGETLSHSHLLLHKKENGIWSTEILKNFKNKTFLKCEAPNYSGRFTCSWLVQRNMDLKENIKSS | linker- |
| | SSSPDSRAVTCGMASLSAEKVTLDQRDYEKYSVSCQEDVTCPTAEETLPIELALEARQQNKYENYST | Fc |
| | SFFIRDIIKPDPPKNLQMKPLKNSQVEVSWEYPDSWSTPHSYFSLKFFVRIQRKKEKMKETEEGCNQ | (RVT- |
| | KGAFLVEKTSTEVQCKGGNVCVQAQDRYYNSSCSKWACVPCRVRS | LALAPG); |
| | HPVGLLARVPLSLYSGHPVGLLARVPLSLYSGLSGRSDNHGGGSGGGS | mIL- |
| | PRGPTIKPCPPCKCPAPNAAGGPSVFIFPPKIKDVLMISLSPIVTCVVVDVSEDDPDVQISWFVNNV | 12- |
| | EVHTA | 2nd |
| | QTQTHREDYNSTLRVVSALPIQHQDWMSGKEFKCKVNNKDLGAPIERTISKPKGSVRAPRVYVLPPP | chain- |
| | EEE | masked: |
| | MTKKQVTLTCMVTDEMPEDIYVEWTNNGKTELNYKNTEPVLVSDGSYTMYSKLRVEKKNWVERNSYS | (972 aa) |
| | CSV VHEGLHNHHTTKSFSRTPG | pro073 |
| SEQ ID | METDTLLLWVLLLWVPGSTG | Signal |
| NO: | RVIPVSGPARCLSQSRNLLKTTDDMVKTAREKLKHYSCTAEDIDHEDITRDQTSTLKTCLPLELH | peptide- |
| 341 | KNESCLATRETSSTTRGSCLPPQKTSLMMTLCLGSIYEDLKMYQTE FQAINAALQNHINHQQIILD | p35- |
| | KGMLVAIDELMQSLNHNGETLRQKPPVGEADPYRVKMKLCILLHAFSTRVVTINRVMGYLSSA | linker- |
| | GGGGSGGGGSGGGGS | Fc- |
| | PRGPTIKPCPPCKCPAPNAAGGPSVFIFPPKIKDVLMISLSPIVTCVVVDVSEDDPDVQISWFVNNV | knob- |
| | EVHTA | EW- |
| | QTQTHREDYNSTLRVVSALPIQHQDWMSGKEFKCKVNNKDLGAPIERTISKPKGSVRAPQVYVLPPP | LALAPG; |
| | EEE | Control |
| | MTEKQVTLTCMVTDEMPEDIYVEWTNNGKTELNYKNTEPVLDSDGSYFMYSWLRVEKKNWVERNSYS | A: |
| | CSV | mIL- |
| | VHEGLHNHHTTKSFSRTPG | 12- |
| | | 1st |
| | | chain: |
| | | (459 aa) |
| | | pro074 |
| SEQ ID | METDTLLLWVLLLWVPGSTG | Signal |
| NO: | MWELEKDVYVVEVDWTPDAPGETVNLTCDTPEEDDITWTSDQRHGVIGSGKTLTITVKEFLDAGQYT | peptide- |
| 342 | CHKGGETLSHSHLLLHKKENGIWSTEILKNFKNKTELKCEAPNYSGRFTCSWLVQRNMDLKENIKSS | p40- |
| | SSSPDSRAVTCGMASLSAEKVTLDQRDYEKYSVSCQEDVTCPTAEETLPIELALEARQQNKYENYST | linker- |
| | SFFIRDIIKPDPPKNLQMKPLKNSQVEVSWEYPDSWSTPHSYFSLKFFVRIQRKKEKMKETEEGCNQ | Fc- |
| | KGAFLVEKTSTEVQCKGGNVCVQAQDRYYNSSCSKWACVPCRVRS | hole- |
| | GGGGSGGGGSGGGGS | RVT- |
| | PRGPTIKPCPPCKCPAPNAAGGPSVFIFPPKIKDVLMISLSPIVTCVVVDVSEDDPDVQISWFVNNV | LALAPG; |
| | EVHTA | Control |
| | QTQTHREDYNSTLRVVSALPIQHQDWMSGKEFKCKVNNKDIGAPIERTISKPKGSVRAPRVYVLPPP | B: |
| | EEE | mIL- |
| | MTKKQVTLTCMVTDEMPEDIYVEWTNNGKTELNYKNTEPVLVSDGSYTMYSKLRVEKKNWVERNSYS | 12- |
| | CSV VHEGLHNHHTTKSFSRTPG | 2nd |
| | | chain: |
| | | (579 aa) |
| | | pro075 |
| SEQ ID | METDTLLLWVLLLWVPGSTG | Signal |
| NO: | QLGASGPGDGCCVEKTSFPEGASGSPLGPRNLSCYRVSKTDYECSWQYDGPEDNVS | peptide- |
| 343 | HVLWCCFVPPNHTHTGQERCRYFSSGPDRTVQFWEQDGIPVLSKVNEWVESRLGNR | Mask |
| | TMKSQKISQYLYNWTKTTPPLGHIKVSQSHRQLRMDWNVSEEAGAEVQERRRMPTT | (IL-12Rb1)- |
| | NWTLGDCGPQVNSGSGVLGDIRGSMSESCLCPSENMAQEIQIRRRRRLSSGAPGGPW | linker-- |
| | SDWSMPVCVPPEVLPQA | p35- |
| | GGGGSGGGGSGGGGS | linker- |
| | RVIPVSGPARCLSQSRNLLKTTDDMVKTAREKLKHYSCTAEDIDHEDITRDQTSTLKTCLPLELH | Fc |
| | KNESCLATRETSSTTRGSCLPPQKTSLMMTLCLGSIYEDLKMYQTEFQAINAALQNHNHQQIILD | (EW- |
| | KGMLVAIDELMQSLNHNGETLRQKPPVGEADPYRVKMKLCILLHAFSTRVVTINRVMGYLSSA | LALAPG); |
| | GGGGSGGGGSGGGGS | Control |
| | PRGPTIKPCPPCKCPAPNAAGGPSVFIFPPKIKDVLMISLSPIVTCVVVDVSEDDPDVQISWFVNNV | C: |
| | EVHTA | mIL- |
| | QTQTHREDYNSTLRVVSALPIQHQDWMSGKEFKCKVNNKDIGAPIERTISKPKGSVRAPQVYVLPPP | 12- |
| | EEE | 1st |
| | MTEKQVTLTCMVTDFMPEDIYVEWTNNGKTELNYKNTEPVLDSDGSYFMYSWLRVEKKNWVERNSYS | chain- |
| | CSV | masked: |
| | VHEGLANHHTTKSFSRTPG | (716 aa) |
| | | pro076 |
| SEQ ID | METDTLLLWVLLLWVPGSTG | Signal |
| NO: | NIDVCKLGTVTVQPAPVIPLGSAANISCSLNPKQGCSHYPSSNELILLKFVNDVLVENLHGKKVHDH | peptide- |
| 344 | TGHSSTFQVTNLSLGMTLFVCKLNCSNSQKKPPVPVCGVEISVGVAPEPPQNISCVQEGENGTVACS | Mask |
| | WNSGKVTYLKTNYTLQLSGPNNLTCQKQCFSDNRQNCNRLDLGINLSPDLAESRFIVRVTAINDLGN | (IL-12Rb2)- |
| | SSSLPHTFTFLDIVIPLPPWDIRINELNASGSRGTLQWEDEGQVVLNQLRYQPLNSTSWNMVNATNA | linker-- |
| | KGKYDLRDLRPFTEYEFQISSKLHLSGGSWSNWSESLRTRTPEE | p40- |
| | GGGGSGGGGSGGGGS | linker- |
| | MWELEKDVYVVEVDWTPDAPGETVNLTCDTPEEDDITWTSDQRHGVIGSGKTLTITVKEFLDAGQYT | Fc |
| | CHKGGETLSHSHLLLHKKENGIWSTEILKNFKNKTFLKCEAPNYSGRFTCSWLVQRNMDLKFNIKSS | (RVT- |
| | SSSPDSRAVTCGMASLSAEKVTLDQRDYEKYSVSCQEDVTCPTAEETLPIELALEARQQNKYENYST | LALAPG); |
| | SFFIRDIIKPDPPKNLQMKPLKNSQVEVSWEYPDSWSTPHSYFSLKFFVRIQRKKEKMKETEEGCNQ | Control |
| | KGAFLVEKTSTEVQCKGGNVCVQAQDRYYNSSCSKWACVPCRVRS | D: |
| | GGGGSGGGGSGGGGS | mIL- |
| | PRGPTIKPCPPCKCPAPNAAGGPSVFIFPPKIKDVLMISLSPIVTCVVVDVSEDDPDVQISWFVNNV | 12- |
| | EVHTA | 2nd |
| | QTQTHREDYNSTLRVVSALPIQHQDWMSGKEFKCKVNNKDLGAPIERTISKPKGSVRAPRVYVLPPP | chain- |
| | EEE | masked: |
| | MTKKQVTLTCMVTDEMPEDIYVEWTNNGKTELNYKNTEPVLVSDGSYTMYSKERVEKKNWVERNSYS | (906 aa) |
| | CSV VHEGLHNHHTTKSFSRTPG | pro077 |
| SEQ ID | MEFGLSWVFLVALFRGVQC | SPH7- |
| NO: | RNLPVATPDPGMFPCLHHSQNLLRAVSNMLQKARQTLEFYPCTSEEIDHEDITKDKTSTVEACLPLE | p35- |
| 321 | LTKNESCLNSRETSFITNGSCLASRKTSFMMALCLSSIYEDLKMYQVEFKTMNAKLLMDPKRQIFLD | cleavable |
| | QNMLAVIDELMQALNENSETVPQKSSLEEPDFYKTKIKLCILLHAFRIRAVTIDRVMSYLNAS | linker- |
| | HPVGLLARVPLSLYSGHPVGLLARVPLSLYSGLSGRSDNHGGGSGGGS | Fc- |
| | PKSSDKTHTCPPCPAPEAAGGPSVFLFPPKPKDTLMISRTPEVTCVVVDVSHEDPEVKENWYVDGVE | EW- |
| | VHNAKTKPREEQYNSTYRVVSVLTVLHQDWINGKEYKCKVSNKALGAPIEKTISKAKGQPREPQVCT | LALAPG |
| | LPPSRDELTENQVSLTCLVKGFYPSDIAVEWESNGQPENNYKTTPPVLDSDGSFFLYSWLTVDKSRW | (aligned |
| | QQGNVFSCSVMHEALHNHYTQKSLSLSPGK | with |
| | | Seq |
| | | 292 |
| | | 230 aa); |
| | | hIL- |
| | | 12- |
| | | 1st |
| | | chain; |
| | | (495 aa) |
| | | pro078 |
| SEQ ID | MEFGLSWVFLVALFRGVQC | SPH7- |
| NO: | IWELKKDVYVVELDWYPDAPGEMVVLTCDTPEEDGITWTLDQSSEVLGSGKTLTIQVKEFGDAGQYT | p40- |
| 321 | CHKGGEVLSHSLLLLHKKEDGIWSTDILKDQKEPKNKTFLRCEAKNYSGRFTCWWLTTISTDLTFSV | cleavable |
| | KSSRGSSDPQGVTCGAATLSAERVRGDNKEYEYSVECQEDSACPAAEESLPIEVMVDAVHKLKYENY | linker- |
| | TSSFFIRDIIKPDPPKNLQLKPLKNSRQVEVSWEYPDTWSTPHSYFSLTFCVQVQGKSKREKKDRVF | hIgG1Fc- |
| | TDKTSATVICRKNASISVRAQDRYYSSSWSEWASVPCS | RVT- |
| | HPVGLLARVPLSLYSGHPVGLLARVPLSLYSGLSGRSDNHGGGSGGGS | LALAPG- |
| | PKSSDKTHTCPPCPAPEAAGGPSVFLFPPKPKDTLMISRTPEVTCVVVDVSHEDPEVKFNWYVDGVE | S354C; |
| | VHNAKTKPREEQYNSTYRVVSVLTVLHQDWLNGKEYKCKVSNKALGAPIEKTISKAKGQPREPRVYT | hIL- |
| | LPPCRDELTKNQVSLTCLVKGFYPSDIAVEWESNGQPENNYKTTPPVLVSDGSFTLYSKLTVDKSRW | 12- |
| | QQGNVFSCSVMHEALHNHYTQKSLSLSPGK | 2nd |
| | | chain: |
| | | (604 aa) |
| | | pro079 |
| SEQ ID | MEFGLSWVFLVALERGVQC | SPH7- |
| NO: | CRTSECCFQDPPYPDADSGSASGPRDLRCYRISSDRYECSWQYEGPTAGVSHFLRCCLSSGRCCYFA | Mask |
| 323 | AGSATRLQFSDQAGVSVLYTVTLWVESWARNQTEKSPEVTLQLYNSVKYEPPLGDIKVSKLAGQLRM | (hIL-12Rb1)-- |
| | EWETPDNQVGAEVQFRHRTPSSPWKLGDCGPQDDDTESCLCPLEMNVAQEFQLRRRQLGSQGSSWSK | cleavable |
| | WSSPVCVPPENP | linker- |
| | HPVGLLARVPLSLYSGHPVGLLARVPLSLYSGLSGRSDNHGGGSGGGS | p35- |
| | RNLPVATPDPGMFPCLHHSQNLLRAVSNMLQKARQTLEFYPCTSEEIDHEDITKDKTSTVEACLPLE | cleavable |
| | LTKNESCINSRETSFITNGSCLASRKTSEMMALCLSSIYEDLKMYQVEFKTMNAKLIMDPKRQIFLD | linker- |
| | QNMLAVIDELMQALNENSETVPQKSSLEEPDEYKTKIKLCILLHAFRIRAVTIDRVMSYLNAS | Fc- |
| | HPVGLLARVPLSLYSGHPVGLLARVPLSLYSGLSGRSDNHGGGSGGGS | EW- |
| | PKSSDKTHTCPPCPAPEAAGGPSVFLFPPKPKDTLMISRTPEVTCVVVDVSHEDPEVKFNWYVDGVE | LALAPG; |
| | VHNAKTKPREEQYNSTYRVVSVLTVLHQDWINGKEYKCKVSNKALGAPIEKTISKAKGQPREPQVCT | hIL |
| | LPPSRDELTENQVSLTCLVKGFYPSDIAVEWESNGQPENNYKTTPPVLDSDGSFFLYSWLTVDKSRW | 12- |
| | QQGNVFSCSVMHEALHNHYTQKSLSLSPGK | 1st |
| | | chain- |
| | | masked: |
| | | (756 aa) |
| | | pro080 |
| SEQ ID | MEFGLSWVFLVALFRGVQC | SPH7- |
| NO: | KIDACKRGDVTVKPSHVILLGSTVNITCSLKPRQGCFHYSRENKLILYKFDRRINFHHGHSLNSQVT | Mask |
| 324 | GLPLGTTLFVCKLACINSDEIQICGAEIFVGVAPEQPQNLSCIQKGEQGTVACTWERGRDTHLYTEY | (hIL-12Rb2)- |
| | TLQLSGPKNLTWQKQCKDIYCDYLDFGINLTPESPESNFTAKVTAVNSLGSSSSLPSTFTFLDIVRP | cleavable |
| | LPPWDIRIKFQKASVSRCTLYWRDEGLVLLNRLRYRPSNSRLWNMVNVTKAKGRHDLLDLKPFTEYE | linker- |
| | FQISSKLHLYKGSWSDWSESLRAQTPEE | p40- |
| | HPVGLLARVPLSLYSGHPVGLLARVPLSLYSGLSGRSDNHGGGSGGGS | cleavable |
| | IWELKKDVYVVELDWYPDAPGEMVVLTCDTPEEDGITWTLDQSSEVLGSGKTLTIQVKEFGDAGQYT | linker- |
| | CHKGGEVLSHSLLLLHKKEDGIWSTDILKDQKEPKNKTFLRCEAKNYSGRFTCWWLTTISTDLTFSV | hIgG1Fc- |
| | KSSRGSSDPQGVTCGAATLSAERVRGDNKEYEYSVECQEDSACPAAEESLPIEVMVDAVHKLKYENY | RVT- |
| | TSSFFIRDIIKPDPPKNLQLKPLKNSRQVEVSWEYPDTWSTPHSYFSLTFCVQVQGKSKREKKDRVF | LALAPG- |
| | TDKTSATVICRKNASISVRAQDRYYSSSWSEWASVPCS | S354C; |
| | HPVGLLARVPLSLYSGHPVGLLARVPLSLYSGLSGRSDNHGGGSGGGS | hIL- |
| | PKSSDKTHTCPPCPAPEAAGGPSVFLFPPKPKDTLMISRTPEVTCVVVDVSHEDPEVKENWYVDGVE | 12- |
| | VHNAKTKPREEQYNSTYRVVSVLTVLHQDWLNGKEYKCKVSNKALGAPIEKTISKAKGQPREPRVYT | 2nd |
| | LPPCRDELTKNQVSLTCLVKGFYPSDIAVEWESNGQPENNYKTTPPVLVSDGSFTLYSKLTVDKSRW | chain- |
| | QQGNVFSCSVMHEALHNHYTQKSLSLSPGK | masked: |
| | | (948 aa) |
| | | pro081 |
| SEQ ID | MEFGLSWVFLVALFRGVQC | SPH7- |
| NO: | RNLPVATPDPGMFPCLHHSQNLLRAVSNMLQKARQTLEFYPCTSEEIDHEDITKDKTSTVEACLPLE | p35; |
| 325 | LTKNESCENSRETSFITNGSCLASRKTSEMMALCLSSIYEDLKMYQVEFKTMNAKLLMDPKRQIFLD | hIL- |
| | QNMLAVIDELMQALNENSETVPQKSSLEEPDFYKTKIKLCILLHAFRIRAVTIDRVMSYLNAS | 12- |
| | | 1st |
| | | chain |
| | | p35: |
| | | (216 aa) |
| | | pro |
| SEQ ID | MEFGLSWVELVALERGVQC | SPH7- |
| NO: | IWELKKDVYVVELDWYPDAPGEMVVLTCDTPEEDGITWTLDQSSEVLGSGKTLTIQVKEFGDAGQYT | p40; |
| 326 | CHKGGEVLSHSLLLLHKKEDGIWSTDILKDQKEPKNKTFLRCEAKNYSGRFTCWWLTTISTDLTESV | hIL- |
| | KSSRGSSDPQGVTCGAATLSAERVRGDNKEYEYSVECQEDSACPAAEESLPIEVMVDAVHKLKYENY | 12- |
| | TSSFFIRDIIKPDPPKNLQLKPLKNSRQVEVSWEYPDTWSTPHSYFSLTFCVQVQGKSKREKKDRVF | 2nd |
| | TDKTSATVICRKNASISVRAQDRYYSSSWSEWASVPCS | chain |
| | | p40: |
| | | (325 aa) |
| | | pro0 |
| SEQ ID | MEFGLSWVFLVALFRGVQC | SPH7- |
| NO: | CRTSECCFQDPPYPDADSGSASGPRDLRCYRISSDRYECSWQYEGPTAGVSHELRCCLSSGRCCYFA | Mask |
| 327 | AGSATRLQFSDQAGVSVLYTVTLWVESWARNQTEKSPEVTLQLYNSVKYEPPLGDIKVSKLAGQLRM | (hIL-12Rb1)-- |
| | EWETPDNQVGAEVQFRHRTPSSPWKLGDCGPQDDDTESCLCPLEMNVAQEFQLRRRQLGSQGSSWSK | cleavable |
| | WSSPVCVPPENP | linker- |
| | HPVGLLARVPLSLYSGHPVGLLARVPLSLYSGLSGRSDNHGGGSGGGS | p35; |
| | RNLPVATPDPGMFPCLHHSQNLLRAVSNMLQKARQTLEFYPCTSEEIDHEDITKDKTSTVEACLPLE | hIL- |
| | LTKNESCENSRETSFITNGSCLASRKTSFMMALCLSSIYEDLKMYQVEFKTMNAKLLMDPKRQIFLD | 12- |
| | QNMLAVIDELMQALNENSETVPQKSSLEEPDFYKTKIKLCILLHAFRIRAVTIDRVMSYLNAS | 1st |
| | | chain- |
| | | masked |
| | | p35: |
| | | (477 aa) |
| | | pro0 |
| SEQ ID | MEFGLSWVFLVALFRGVQC | SPH7- |
| NO: | KIDACKRGDVTVKPSHVILLGSTVNITCSLKPRQGCFHYSRENKLILYKFDRRINEHHGHSLNSQVT | Mask |
| 328 | GLPLGTTLEVCKLACINSDEIQICGAEIFVGVAPEQPQNLSCIQKGEQGTVACTWERGRDTHLYTEY | (hIL-12Rb2)- |
| | TLQLSGPKNLTWQKQCKDIYCDYLDFGINLTPESPESNFTAKVTAVNSLGSSSSLPSTFTFLDIVRP | cleavable |
| | LPPWDIRIKFQKASVSRCTLYWRDEGLVLLNRLRYRPSNSRLWNMVNVTKAKGRHDLLDLKPFTEYE | linker- |
| | FQISSKLHLYKGSWSDWSESLRAQTPEE | p40; |
| | HPVGLLARVPLSLYSGHPVGLLARVPLSLYSGLSGRSDNHGGGSGGGS | hIL- |
| | IWELKKDVYVVELDWYPDAPGEMVVLTCDTPEEDGITWTLDQSSEVLGSGKTLTIQVKEFGDAGQYT | 12- |
| | CHKGGEVLSHSLLLLHKKEDGIWSTDILKDQKEPKNKTFLRCEAKNYSGRFTCWWLTTISTDLTFSV | 2nd |
| | KSSRGSSDPQGVTCGAATLSAERVRGDNKEYEYSVECQEDSACPAAEESLPIEVMVDAVHKLKYENY | chain- |
| | TSSFFIRDIIKPDPPKNLQLKPLKNSRQVEVSWEYPDTWSTPHSYFSLTFCVQVQGKSKREKKDRVE | masked |
| | TDKTSATVICRKNASISVRAQDRYYSSSWSEWASVPCS | p40: |
| | | (669 aa) |
| | | pro081 |
| SEQ ID | MCPQKLTISWEAIVLLVSPLMAMWELEKDVYVVEVDWTPDAPGETVNLTCDTPEEDDITWTSDQRHG | IL-12 |
| NO: | VIGSGKTLTITVKEFLDAGQYTCHKGGETLSHSHLLLHKKENGIWSTEILKNEKNKTFLKCEAPNYS | p35- |
| 483 | GRFTCSWLVQRNMDLKFNIKSSSSSPDSRAVTCGMASLSAEKVTLDQRDYEKYSVSCQEDVTCPTAE | IgG |
| | ETLPIELALEARQQNKYENYSTSFFIRDIIKPDPPKNLQMKPLKNSQVEVSWEYPDSWSTPHSYFSL | |
| | KFFVRIQRKKEKMKETEEGCNQKGAFLVEKTSTEVQCKGGNVCVQAQDRYYNSSCSKWACVPCRVRS | |
| | GGGGSGGGGSGGGGSRVIPVSGPARCLSQSRNLLKTTDDMVKTAREKLKHYSCTAEDIDHEDITRDQ | |
| | TSTLKTCLPLELHKNESCLATRETSSTTRGSCLPPQKTSLMMTLCLGSIYEDLKMYQTEFQAINAAL | |
| | QNHNHQQIILDKGMLVAIDELMQSLNHNGETLRQKPPVGEADPYRVKMKLCILLHAFSTRVVTINRV | |
| | MGYLSSASGGGSGGGGSGGGGSGGGGSGGGSLQVVISAILALVVLTVISLIILIGGGGSGKPIPNPL | |
| | LGLDST | |
| SEQ ID | MEFGLSWVFLVALFRGVQCRNLPVATPDPGMFPCLHHSQNLLRAVSNMLQKARQTLEFYPCTSEEID | IL-12 |
| NO: | HEDITKDKTSTVEACLPLELTKNESCLNSRETSFITNGSCLASRKTSEMMALCLSSIYEDLKMYQVE | p35- |
| 484 | FKTMNAKLLMDPKRQIFLDQNMLAVIDELMQALNFNSETVPQKSSLEEPDFYKTKIKLCILLHAFRI | IgG |
| | RAVTIDRVMSYLNASHPVGLLARVPLSLYSGHPVGLLARVPLSLYSGLSGRSDNHGGGSGGGSPKSS | |
| | DKTHTCPPCPAPEAAGGPSVFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALGAPIEKTISKAKGQPREPQVCTLPPS | |
| | RDELTENQVSLTCLVKGFYPSDIAVEWESNGQPENNYKTTPPVLDSDGSFFLYSWLTVDKSRWQQGN | |
| | VFSCSVMHEALHNHYTQKSLSLSPGK | |
| SEQ ID | MEFGLSWVFLVALFRGVQCIWELKKDVYVVELDWYPDAPGEMVVLTCDTPEEDGITWTLDQSSEVLG | IL-12 |
| NO: | SGKTLTIQVKEFGDAGQYTCHKGGEVLSHSLLLLHKKEDGIWSTDILKDQKEPKNKTFLRCEAKNYS | p35- |
| 485 | GRFTCWWLTTISTDLTFSVKSSRGSSDPQGVTCGAATLSAERVRGDNKEYEYSVECQEDSACPAAEE | IgG |
| | SLPIEVMVDAVHKLKYENYTSSFFIRDIIKPDPPKNLQLKPLKNSRQVEVSWEYPDTWSTPHSYESL | |
| | TFCVQVQGKSKREKKDRVFTDKTSATVICRKNASISVRAQDRYYSSSWSEWASVPCSHPVGLLARVP | |
| | LSLYSGHPVGLLARVPLSLYSGLSGRSDNHGGGSGGGSPKSSDKTHTCPPCPAPEAAGGPSVFLFPP | |
| | KPKDTLMISRTPEVTCVVVDVSHEDPEVKFNWYVDGVEVHNAKTKPREEQYNSTYRVVSVLTVLHQD | |
| | WLNGKEYKCKVSNKALGAPIEKTISKAKGQPREPRVYTLPPCRDELTKNQVSLTCLVKGFYPSDIAV | |
| | EWESNGQPENNYKTTPPVLVSDGSFTLYSKLTVDKSRWQQGNVFSCSVMHEALHNHYTQKSLSLSPG | |
| | K | |
| SEQ ID | MEFGLSWVFLVALFRGVQCCRTSECCFQDPPYPDADSGSASGPRDLRCYRISSDRYECSWQYEGPTA | IL-12 |
| NO: | GVSHFLRCCLSSGRCCYFAAGSATRLQFSDQAGVSVLYTVTLWVESWARNQTEKSPEVTLQLYNSVK | p35- |
| 486 | YEPPLGDIKVSKLAGQLRMEWETPDNQVGAEVQFRHRTPSSPWKLGDCGPQDDDTESCLCPLEMNVA | IgG |
| | QEFQLRRRQLGSQGSSWSKWSSPVCVPPENPHPVGLLARVPLSLYSGHPVGLLARVPLSLYSGLSGR | |
| | SDNHGGGSGGGSRNLPVATPDPGMFPCLHHSQNLLRAVSNMLQKARQTLEFYPCTSEEIDHEDITKD | |
| | KTSTVEACLPLELTKNESCLNSRETSFITNGSCLASRKTSFMMALCLSSIYEDLKMYQVEFKTMNAK | |
| | LLMDPKRQIFLDQNMLAVIDELMQALNENSETVPQKSSLEEPDFYKTKIKLCILLHAFRIRAVTIDR | |
| | VMSYLNASHPVGLLARVPLSLYSGHPVGLLARVPLSLYSGLSGRSDNHGGGSGGGSPKSSDKTHTCP | |
| | PCPAPEAAGGPSVFLFPPKPKDTLMISRTPEVTCVVVDVSHEDPEVKFNWYVDGVEVHNAKTKPREE | |
| | QYNSTYRVVSVLTVLHQDWLNGKEYKCKVSNKALGAPIEKTISKAKGQPREPQVCTLPPSRDELTEN | |
| | QVSLTCLVKGFYPSDIAVEWESNGQPENNYKTTPPVLDSDGSFFLYSWLTVDKSRWQQGNVFSCSVM | |
| | HEALHNHYTQKSLSLSPGK | |
| SEQ ID | MEFGLSWVFLVALFRGVQCKIDACKRGDVTVKPSHVILLGSTVNITCSIKPRQGCFHYSRRNKLILY | IL-12 |
| NO: | KFDRRINFHHGHSLNSQVTGLPLGTTLFVCKLACINSDEIQICGAEIFVGVAPEQPQNLSCIQKGEQ | p35- |
| 487 | GTVACTWERGRDTHLYTEYTLQLSGPKNLTWQKQCKDIYCDYLDFGINLTPESPESNFTAKVTAVNS | IgG |
| | LGSSSSLPSTFTFLDIVRPLPPWDIRIKFQKASVSRCTLYWRDEGLVLLNRLRYRPSNSRLWNMVNV | |
| | TKAKGRHDLLDLKPFTEYEFQISSKLHLYKGSWSDWSESLRAQTPEEHPVGLLARVPLSLYSGHPVG | |
| | LLARVPLSLYSGLSGRSDNHGGGSGGGSIWELKKDVYVVELDWYPDAPGEMVVLTCDTPEEDGITWT | |
| | LDQSSEVLGSGKTLTIQVKEFGDAGQYTCHKGGEVLSHSLLLLHKKEDGIWSTDILKDQKEPKNKTE | |
| | LRCEAKNYSGRFTCWWLTTISTDLTFSVKSSRGSSDPQGVTCGAATLSAERVRGDNKEYEYSVECQE | |
| | DSACPAAEESLPIEVMVDAVHKLKYENYTSSFFIRDIIKPDPPKNLQLKPLKNSRQVEVSWEYPDTW | |
| | STPHSYFSLTFCVQVQGKSKREKKDRVFTDKTSATVICRKNASISVRAQDRYYSSSWSEWASVPCSH | |
| | PVGLLARVPLSLYSGHPVGLLARVPLSLYSGLSGRSDNHGGGSGGGSPKSSDKTHTCPPCPAPEAAG | |
| | GPSVFLFPPKPKDTLMISRTPEVTCVVVDVSHEDPEVKFNWYVDGVEVHNAKTKPREEQYNSTYRVV | |
| | SVLTVLHQDWLNGKEYKCKVSNKALGAPIEKTISKAKGQPREPRVYTLPPCRDELTKNQVSLTCLVK | |
| | GFYPSDIAVEWESNGQPENNYKTTPPVLVSDGSFTLYSKLTVDKSRWQQGNVFSCSVMHEALHNHYT | |
| | QKSLSLSPGK | |
| SEQ ID | MEFGLSWVFLVALFRGVQCRNLPVATPDPGMFPCLHHSQNLLRAVSNMLQKARQTLEFYPCTSEEID | Pro078 |
| NO: | HEDITKDKTSTVEACLPLELTKNESCLNSRETSFITNGSCLASRKTSEMMALCLSSIYEDLKMYQVE | |
| 648 | FKTMNAKLLMDPKRQIELDQNMLAVIDELMQALNENSETVPQKSSLEEPDFYKTKIKLCILLHAFRI | |
| | RAVTIDRVMSYLNASHPVGLLARVPLSLYSGHPVGLLARVPLSLYSGLSGRSDNHGGGSGGGSPKSS | |
| | DKTHTCPPCPAPEAAGGPSVFLFPPKPKDELMISRTPEVTCVVVDVSHEDPEVKFNWYVDGVEVHNA | |
| | KTKPREEQYNSTYRVVSVLTVLHQDWLNGKEYKCKVSNKALGAPIEKTISKAKGQPREPQVCTLPPS | |
| | RDELTENQVSLTCLVKGFYPSDIAVEWESNGQPENNYKTTPPVLDSDGSFFLYSWLTVDKSRWQQGN | |
| | VFSCSVMHEALHNHYTQKSLSLSPGK | |
| SEQ ID | MEFGLSWVELVALFRGVQCIWELKKDVYVVELDWYPDAPGEMVVLTCDTPEEDGITWTLDQSSEVLG | Pro079 |
| NO: | SGKTLTIQVKEFGDAGQYTCHKGGEVLSHSLLLLHKKEDGIWSTDILKDQKEPKNKTFLRCEAKNYS | |
| 649 | GRFTCWWLTTISTDLTFSVKSSRGSSDPQGVTCGAATLSAERVRGDNKEYEYSVECQEDSACPAAEE | |
| | SLPIEVMVDAVHKLKYENYTSSFFIRDIIKPDPPKNLQLKPLKNSRQVEVSWEYPDTWSTPHSYFSL | |
| | TFCVQVQGKSKREKKDRVFTDKTSATVICRKNASISVRAQDRYYSSSWSEWASVPCSHPVGLLARVP | |
| | LSLYSGHPVGLLARVPLSLYSGLSGRSDNHGGGSGGGSPKSSDKTHTCPPCPAPEAAGGPSVFLFPP | |
| | KPKDTLMISRTPEVTCVVVDVSHEDPEVKFNWYVDGVEVHNAKTKPREEQYNSTYRVVSVLTVLHQD | |
| | WLNGKEYKCKVSNKALGAPIEKTISKAKGQPREPRVYTLPPCRDELTKNQVSLTCLVKGFYPSDIAV | |
| | EWESNGQPENNYKTTPPVLVSDGSFTLYSKLTVDKSRWQQGNVFSCSVMHEALHNHYTQKSLSLSPG | |
| | K | |
| SEQ ID | MEFGLSWVFLVALFRGVQCRNLPVATPDPGMFPCLHHSQNLLRAVSNMLQKARQTLEFYPCTSEEID | Pro078 |
| NO: | HEDITKDKTSTVEACLPLELTKNESCLNSRETSFITNGSCLASRKTSEMMALCLSSIYEDLKMYQVE | |
| 650 | FKTMNAKLLMDPKRQIFLDQNMLAVIDELMQALNENSETVPQKSSLEEPDFYKTKIKLCILLHAFRI | |
| | RAVTIDRVMSYLNASHPVGLLARVPLSLYSGHPVGLLARVPLSLYSGLSGRSDNHGGGSGGGSPKSS | |
| | DKTHTCPPCPAPEAAGGPSVFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALGAPIEKTISKAKGQPREPQVCTLPPS | |
| | RDELTENQVSLTCLVKGFYPSDIAVEWESNGQPENNYKTTPPVLDSDGSFFLYSWLTVDKSRWQQGN | |
| | VFSCSVMHEALHNHYTQKSLSLSPGK | |
| SEQ ID | MEFGLSWVFLVALFRGVQCCRTSECCFQDPPYPDADSGSASGPRDLRCYRISSDRYECSWQYEGPTA | Pro080 |
| NO: | GVSHELRCCLSSGRCCYFAAGSATRLQFSDQAGVSVLYTVTLWVESWARNQTEKSPEVTLQLYNSVK | |
| 651 | YEPPLGDIKVSKLAGQLRMEWETPDNQVGAEVQFRHRTPSSPWKLGDCGPQDDDTESCLCPLEMNVA | |
| | QEFQLRRRQLGSQGSSWSKWSSPVCVPPENPHPVGLLARVPLSLYSGHPVGLLARVPLSLYSGLSGR | |
| | SDNHGGGSGGGSRNLPVATPDPGMFPCLHHSQNLLRAVSNMLQKARQTLEFYPCTSEEIDHEDITKD | |
| | KTSTVEACLPLELTKNESCLNSRETSFITNGSCLASRKTSEMMALCLSSIYEDLKMYQVEFKTMNAK | |
| | LLMDPKRQIFLDQNMLAVIDELMQALNENSETVPQKSSLEEPDFYKTKIKLCILLHAFRIRAVTIDR | |
| | VMSYLNASHPVGLLARVPLSLYSGHPVGLLARVPLSLYSGLSGRSDNHGGGSGGGSPKSSDKTHTCP | |
| | PCPAPEAAGGPSVFLFPPKPKDTLMISRTPEVTCVVVDVSHEDPEVKFNWYVDGVEVHNAKTKPREE | |
| | QYNSTYRVVSVLTVLHQDWLNGKEYKCKVSNKALGAPIEKTISKAKGQPREPQVCTLPPSRDELTEN | |
| | QVSLTCLVKGFYPSDIAVEWESNGQPENNYKTTPPVLDSDGSFFLYSWLTVDKSRWQQGNVFSCSVM | |
| | HEALHNHYTQKSLSLSPGK | |
| SEQ ID | METDTLLLWVLLLWVPGSTGMWELEKDVYVVEVDWTPDAPGETVNLTCDTPEEDDITWTSDQRHGVI | PRO071 |
| NO: | GSGKTLTITVKEFLDAGQYTCHKGGETLSHSHLLLHKKENGIWSTEILKNFKNKTELKCEAPNYSGR | |
| 652 | FTCSWLVQRNMDLKFNIKSSSSSPDSRAVTCGMASLSAEKVTLDQRDYEKYSVSCQEDVTCPTAEET | |
| | LPIELALEARQQNKYENYSTSFFIRDIIKPDPPKNLQMKPLKNSQVEVSWEYPDSWSTPHSYFSLKF | |
| | FVRIQRKKEKMKETEEGCNQKGAFLVEKTSTEVQCKGGNVCVQAQDRYYNSSCSKWACVPCRVRSHP | |
| | VGLLARVPLSLYSGHPVGLLARVPLSLYSGLSGRSDNHGGGSGGGSPRGPTIKPCPPCKCPAPNAAG | |
| | GPSVFIFPPKIKDVLMISLSPIVTCVVVDVSEDDPDVQISWFVNNVEVHTAQTQTHREDYNSTLRVV | |
| | SALPIQHQDWMSGKEFKCKVNNKDLGAPIERTISKPKGSVRAPRVYVLPPPEEEMTKKQVTLTCMVT | |
| | DEMPEDIYVEWTNNGKTELNYKNTEPVLVSDGSYTMYSKLRVEKKNWVERNSYSCSVVHEGLHNHHT | |
| | TKSFSRTPG | |
| SEQ ID | METDTLLLWVLLLWVPGSTGQLGASGPGDGCCVEKTSFPEGASGSPLGPRNLSCYRVSKTDYECSWQ | PRO072 |
| NO: | YDGPEDNVSHVLWCCFVPPNHTHTGQERCRYESSGPDRTVQFWEQDGIPVLSKVNEWVESRLGNRTM | |
| 653 | KSQKISQYLYNWTKTTPPLGHIKVSQSHRQLRMDWNVSEEAGAEVQFRRRMPTTNWTLGDCGPQVNS | |
| | GSGVLGDIRGSMSESCLCPSENMAQEIQIRRRRRLSSGAPGGPWSDWSMPVCVPPEVLPQAHPVGLL | |
| | ARVPLSLYSGHPVGLLARVPLSLYSGLSGRSDNHGGGSGGGSRVIPVSGPARCLSQSRNLLKTTDDM | |
| | VKTAREKLKHYSCTAEDIDHEDITRDQTSTLKTCLPLELHKNESCLATRETSSTTRGSCLPPQKTSL | |
| | MMTLCLGSIYEDIKMYQTEFQAINAALQNHNHQQIILDKGMLVAIDELMQSLNHNGETLRQKPPVGE | |
| | ADPYRVKMKLCILLHAFSTRVVTINRVMGYLSSAHPVGLLARVPLSLYSGHPVGLLARVPLSLYSGL | |
| | SGRSDNHGGGSGGGSPRGPTIKPCPPCKCPAPNAAGGPSVFIFPPKIKDVLMISLSPIVTCVVVDVS | |
| | EDDPDVQISWFVNNVEVHTAQTQTHREDYNSTLRVVSALPIQHQDWMSGKEFKCKVNNKDLGAPIER | |
| | TISKPKGSVRAPQVYVLPPPEEEMTEKQVTLTCMVTDEMPEDIYVEWTNNGKTELNYKNTEPVLDSD | |
| | GSYFMYSWIRVEKKNWVERNSYSCSVVHEGLHNHHTTKSFSRTPG | |
| SEQ ID | METDTLLLWVLLLWVPGSTGNIDVCKLGTVTVQPAPVIPLGSAANISCSLNPKQGCSHYPSSNELIL | PRO073 |
| NO: | LKFVNDVLVENLHGKKVHDHTGHSSTFQVTNLSLGMTLFVCKLNCSNSQKKPPVPVCGVEISVGVAP | |
| 654 | EPPQNISCVQEGENGTVACSWNSGKVTYLKTNYTLQLSGPNNLTCQKQCFSDNRQNCNRLDLGINES | |
| | PDLAESRFIVRVTAINDLGNSSSLPHTFTFLDIVIPLPPWDIRINELNASGSRGTLQWEDEGQVVLN | |
| | QLRYQPLNSTSWNMVNATNAKGKYDLRDLRPFTEYEFQISSKLHLSGGSWSNWSESLRTRTPEEHPV | |
| | GLLARVPLSLYSGHPVGLLARVPLSLYSGLSGRSDNHGGGSGGGSMWELEKDVYVVEVDWTPDAPGE | |
| | TVNLTCDTPEEDDITWTSDQRHGVIGSGKTLTITVKEFLDAGQYTCHKGGETLSHSHLLLHKKENGI | |
| | WSTEILKNEKNKTFLKCEAPNYSGRFTCSWLVQRNMDLKENIKSSSSSPDSRAVTCGMASLSAEKVT | |
| | LDQRDYEKYSVSCQEDVTCPTAEETLPIELALEARQQNKYENYSTSFFIRDIIKPDPPKNLQMKPLK | |
| | NSQVEVSWEYPDSWSTPHSYFSLKFFVRIQRKKEKMKETEEGCNQKGAFLVEKTSTEVQCKGGNVCV | |
| | QAQDRYYNSSCSKWACVPCRVRSHPVGLLARVPLSLYSGHPVGLLARVPLSLYSGLSGRSDNHGGGS | |
| | GGGSPRGPTIKPCPPCKCPAPNAAGGPSVFIFPPKIKDVLMISLSPIVTCVVVDVSEDDPDVQISWE | |
| | VNNVEVHTAQTQTHREDYNSTLRVVSALPIQHQDWMSGKEFKCKVNNKDLGAPIERTISKPKGSVRA | |
| | PRVYVLPPPEEEMTKKQVTLTCMVTDFMPEDIYVEWTNNGKTELNYKNTEPVLVSDGSYTMYSKLRV | |
| | EKKNWVERNSYSCSVVHEGLHINHHTTKSFSRTPG | |
| SEQ ID | METDTLLLWVLLLWVPGSTGNIDVCKLGTVTVQPAPVIPLGSAANISCSLNPKQGCSHYPSSNELIL | PRO073 |
| NO: | LKFVNDVLVENLHGKKVHDHTGHSSTFQVTNLSLGMTLFVCKINCSNSQKKPPVPVCGVEISVGVAP | |
| 655 | EPPQNISCVQEGENGTVACSWNSGKVTYLKTNYTLQLSGPNNLTCQKQCESDNRQNCNRLDLGINLS | |
| | PDLAESRFIVRVTAINDLGNSSSLPHTFTELDIVIPLPPWDIRINELNASGSRGTLQWEDEGQVVLN | |
| | QLRYQPLNSTSWNMVNATNAKGKYDLRDLRPFTEYEFQISSKLHLSGGSWSNWSESLRTRTPEEHPV | |
| | GLLARVPLSLYSGHPVGLLARVPLSLYSGLSGRSDNHGGGSGGGSMWELEKDVYVVEVDWTPDAPGE | |
| | TVNLTCDTPEEDDITWTSDQRHGVIGSGKTLTITVKEFLDAGQYTCHKGGETLSHSHLLLHKKENGI | |
| | WSTEILKNFKNKTFLKCEAPNYSGRFTCSWLVQRNMDLKFNIKSSSSSPDSRAVTCGMASLSAEKVT | |
| | LDQRDYEKYSVSCQEDVTCPTAEETLPIELALEARQQNKYENYSTSFFIRDIIKPDPPKNLQMKPLK | |
| | NSQVEVSWEYPDSWSTPHSYFSLKFFVRIQRKKEKMKETEEGCNQKGAFLVEKTSTEVQCKGGNVCV | |
| | QAQDRYYNSSCSKWACVPCRVRSHPVGLLARVPLSLYSGHPVGLLARVPLSLYSGLSGRSDNHGGGS | |
| | GGGSPRGPTIKPCPPCKCPAPNAAGGPSVFIFPPKIKDVLMISLSPIVTCVVVDVSEDDPDVQISWF | |
| | VNNVEVHTAQTQTHREDYNSTLRVVSALPIQHQDWMSGKEFKCKVNNKDLGAPIERTISKPKGSVRA | |
| | PRVYVLPPPEEEMTKKQVTLTCMVTDFMPEDIYVEWTNNGKTELNYKNTEPVLVSDGSYTMYSKLRV | |
| | EKKNWVERNSYSCSVVHEGLHNHHTTKSFSRTPG | |
| SEQ ID | METDTLLLWVLLLWVPGSTGMWELEKDVYVVEVDWTPDAPGETVNLTCDTPEEDDITWTSDQRHGVI | PRO075 |
| NO: | GSGKTLTITVKEFLDAGQYTCHKGGETLSHSHLLLHKKENGIWSTEILKNFKNKTFLKCEAPNYSGR | |
| 656 | FTCSWLVQRNMDLKFNIKSSSSSPDSRAVTCGMASLSAEKVTLDQRDYEKYSVSCQEDVTCPTAEET | |
| | LPIELALEARQQNKYENYSTSFFIRDIIKPDPPKNLQMKPLKNSQVEVSWEYPDSWSTPHSYFSLKF | |
| | FVRIQRKKEKMKETEEGCNQKGAFLVEKTSTEVQCKGGNVCVQAQDRYYNSSCSKWACVPCRVRSGG | |
| | GGSGGGGSGGGGSPRGPTIKPCPPCKCPAPNAAGGPSVFIFPPKIKDVLMISLSPIVTCVVVDVSED | |
| | DPDVQISWFVNNVEVHTAQTQTHREDYNSTLRVVSALPIQHQDWMSGKEFKCKVNNKDLGAPIERTI | |
| | SKPKGSVRAPRVYVLPPPEEEMTKKQVTLTCMVTDEMPEDIYVEWTNNGKTELNYKNTEPVLVSDGS | |
| | YTMYSKLRVEKKNWVERNSYSCSVVHEGLHNHHTTKSFSRTPG | |
| SEQ ID | METDTLLLWVLLLWVPGSTGNIDVCKLGTVTVQPAPVIPLGSAANISCSLNPKQGCSHYPSSNELIL | PRO077 |
| NO: | LKFVNDVLVENLHGKKVHDHTGHSSTFQVTNLSLGMTLFVCKLNCSNSQKKPPVPVCGVEISVGVAP | |
| 657 | EPPQNISCVQEGENGTVACSWNSGKVTYLKTNYTLQLSGPNNLTCQKQCFSDNRQNCNRLDLGINLS | |
| | PDLAESRFIVRVTAINDLGNSSSLPHTFTFLDIVIPLPPWDIRINELNASGSRGTLQWEDEGQVVLN | |
| | QLRYQPLNSTSWNMVNATNAKGKYDLRDLRPFTEYEFQISSKLHLSGGSWSNWSESLRTRTPEEGGG | |
| | GSGGGGSGGGGSMWELEKDVYVVEVDWTPDAPGETVNITCDTPEEDDITWTSDQRHGVIGSGKTLTI | |
| | TVKEFLDAGQYTCHKGGETLSHSHLLLHKKENGIWSTEILKNFKNKTFLKCEAPNYSGRETCSWLVQ | |
| | RNMDLKFNIKSSSSSPDSRAVTCGMASLSAEKVELDQRDYEKYSVSCQEDVTCPTAEETLPIELALE | |
| | ARQQNKYENYSTSFFIRDIIKPDPPKNLQMKPLKNSQVEVSWEYPDSWSTPHSYFSLKFFVRIQRKK | |
| | EKMKETEEGCNQKGAFLVEKTSTEVQCKGGNVCVQAQDRYYNSSCSKWACVPCRVRSGGGGSGGGGS | |
| | GGGGSPRGPTIKPCPPCKCPAPNAAGGPSVFIFPPKIKDVLMISLSPIVTCVVVDVSEDDPDVQISW | |
| | FVNNVEVHTAQTQTHREDYNSTLRVVSALPIQHQDWMSGKEFKCKVNNKDLGAPIERTISKPKGSVR | |
| | APRVYVLPPPEEEMTKKQVTLTCMVTDEMPEDIYVEWTNNGKTELNYKNTEPVLVSDGSYTMYSKLR | |
| | VEKKNWVERNSYSCSVVHEGLHNHHTTKSFSRTPG | |
| SEQ ID | METDTLLLWVLLLWVPGSTGMWELEKDVYVVEVDWTPDAPGETVNLTCDTPEEDDITWTSDQRHGVI | PRO075 |
| NO: | GSGKTLTITVKEFLDAGQYTCHKGGETLSHSHLLLHKKENGIWSTEILKNFKNKTFLKCEAPNYSGR | |
| 658 | FTCSWLVQRNMDLKFNIKSSSSSPDSRAVTCGMASLSAEKVTLDQRDYEKYSVSCQEDVTCPTAEET | |
| | LPIELALEARQQNKYENYSTSFFIRDIIKPDPPKNLQMKPLKNSQVEVSWEYPDSWSTPHSYFSLKF | |
| | FVRIQRKKEKMKETEEGCNQKGAFLVEKTSTEVQCKGGNVCVQAQDRYYNSSCSKWACVPCRVRSGG | |
| | GGSGGGGSGGGGSPRGPTIKPCPPCKCPAPNAAGGPSVFIFPPKIKDVLMISLSPIVTCVVVDVSED | |
| | DPDVQISWFVNNVEVHTAQTQTHREDYNSTLRVVSALPIQHQDWMSGKEFKCKVNNKDLGAPIERTI | |
| | SKPKGSVRAPRVYVLPPPEEEMTKKQVTLTCMVTDEMPEDIYVEWTNNGKTELNYKNTEPVLVSDGS | |
| | YTMYSKLRVEKKNWVERNSYSCSVVHEGLHNHHTTKSFSRTPG | |
| SEQ ID | METDTLLLWVLLLWVPGSTGNIDVCKLGTVTVQPAPVIPLGSAANISCSLNPKQGCSHYPSSNELIL | PRO077 |
| NO: | LKFVNDVLVENLHGKKVHDHTGHSSTFQVTNLSLGMTLFVCKLNCSNSQKKPPVPVCGVEISVGVAP | |
| 659 | EPPQNISCVQEGENGTVACSWNSGKVTYLKTNYTLQLSGPNNLTCQKQCFSDNRQNCNRLDLGINLS | |
| | PDLAESRFIVRVTAINDLGNSSSLPHTFTELDIVIPLPPWDIRINELNASGSRGTLQWEDEGQVVLN | |
| | QLRYQPLNSTSWNMVNATNAKGKYDLRDLRPFTEYEFQISSKLHLSGGSWSNWSESLRTRTPEEGGG | |
| | GSGGGGSGGGGSMWELEKDVYVVEVDWTPDAPGETVNLTCDTPEEDDITWTSDQRHGVIGSGKTLTI | |
| | TVKEFLDAGQYTCHKGGETLSHSHLLLHKKENGIWSTEILKNFKNKTFLKCEAPNYSGRETCSWLVQ | |
| | RNMDLKFNIKSSSSSPDSRAVTCGMASLSAEKVTLDQRDYEKYSVSCQEDVTCPTAEETLPIELALE | |
| | ARQQNKYENYSTSFFIRDIIKPDPPKNLQMKPLKNSQVEVSWEYPDSWSTPHSYFSLKFFVRIQRKK | |
| | EKMKETEEGCNQKGAFLVEKTSTEVQCKGGNVCVQAQDRYYNSSCSKWACVPCRVRSGGGGSGGGGS | |
| | GGGGSPRGPTIKPCPPCKCPAPNAAGGPSVFIFPPKIKDVLMISLSPIVTCVVVDVSEDDPDVQISW | |
| | FVNNVEVHTAQTQTHREDYNSTLRVVSALPIQHQDWMSGKEFKCKVNNKDLGAPIERTISKPKGSVR | |
| | APRVYVLPPPEEEMTKKQVTLTCMVTDEMPEDIYVEWTNNGKTELNYKNTEPVLVSDGSYTMYSKLR | |
| | VEKKNWVERNSYSCSVVHEGLHNHHTTKSFSRTPG | |
| SEQ ID | MEFGLSWVFLVALERGVQCIWELKKDVYVVELDWYPDAPGEMVVLTCDTPEEDGITWTLDQSSEVLG | PRO079 |
| NO: | SGKTLTIQVKEFGDAGQYTCHKGGEVLSHSLLLLHKKEDGIWSTDILKDQKEPKNKTFLRCEAKNYS | |
| 660 | GRFTCWWLTTISTDLTFSVKSSRGSSDPQGVTCGAATLSAERVRGDNKEYEYSVECQEDSACPAAEE | |
| | SLPIEVMVDAVHKLKYENYTSSFFIRDIIKPDPPKNLQLKPLKNSRQVEVSWEYPDTWSTPHSYESL | |
| | TFCVQVQGKSKREKKDRVFTDKTSATVICRKNASISVRAQDRYYSSSWSEWASVPCSHPVGLLARVP | |
| | LSLYSGHPVGLLARVPLSLYSGLSGRSDNHGGGSGGGSPKSSDKTHTCPPCPAPEAAGGPSVFLFPP | |
| | KPKDTLMISRTPEVTCVVVDVSHEDPEVKFNWYVDGVEVHNAKTKPREEQYNSTYRVVSVLTVLHQD | |
| | WLNGKEYKCKVSNKALGAPIEKTISKAKGQPREPRVYTLPPCRDELTKNQVSLTCLVKGFYPSDIAV | |
| | EWESNGQPENNYKTTPPVLVSDGSFTLYSKLTVDKSRWQQGNVESCSVMHEALHNHYTQKSLSLSPG | |
| | K | |
| SEQ ID | MEFGLSWVFLVALFRGVQCCRTSECCFQDPPYPDADSGSASGPRDLRCYRISSDRYECSWQYEGPTA | PRO080 |
| NO: | GVSHFLRCCLSSGRCCYFAAGSATRLQFSDQAGVSVLYTVTLWVESWARNQTEKSPEVTLQLYNSVK | |
| 661 | YEPPLGDIKVSKLAGQLRMEWETPDNQVGAEVQFRHRTPSSPWKLGDCGPQDDDTESCLCPLEMNVA | |
| | QEFQLRRRQLGSQGSSWSKWSSPVCVPPENPHPVGLLARVPLSLYSGHPVGLLARVPLSLYSGLSGR | |
| | SDNHGGGSGGGSRNLPVATPDPGMFPCLHHSQNLLRAVSNMLQKARQTLEFYPCTSEEIDHEDITKD | |
| | KTSTVEACLPLELTKNESCLNSRETSFITNGSCLASRKTSEMMALCLSSIYEDLKMYQVEFKTMNAK | |
| | LLMDPKRQIFLDQNMLAVIDELMQALNENSETVPQKSSLEEPDFYKTKIKLCILLHAFRIRAVTIDR | |
| | VMSYLNASHPVGLLARVPLSLYSGHPVGLLARVPLSLYSGLSGRSDNHGGGSGGGSPKSSDKTHTCP | |
| | PCPAPEAAGGPSVFLFPPKPKDTLMISRTPEVTCVVVDVSHEDPEVKFNWYVDGVEVHNAKTKPREE | |
| | QYNSTYRVVSVLTVLHQDWLNGKEYKCKVSNKALGAPIEKTISKAKGQPREPQVCTLPPSRDELTEN | |
| | QVSLTCLVKGFYPSDIAVEWESNGQPENNYKTTPPVLDSDGSFFLYSWLTVDKSRWQQGNVESCSVM | |
| | HEALHNHYTQKSLSLSPGK | |
| SEQ ID | MEFGLSWVELVALFRGVQCKIDACKRGDVTVKPSHVILLGSTVNITCSLKPRQGCFHYSRENKLILY | PRO081 |
| NO: | KFDRRINFHHGHSLNSQVTGLPLGTTLFVCKLACINSDEIQICGAEIFVGVAPEQPQNLSCIQKGEQ | |
| 662 | GTVACTWERGRDTHLYTEYTLQLSGPKNLTWQKQCKDIYCDYLDFGINLTPESPESNFTAKVTAVNS | |
| | LGSSSSLPSTFTFLDIVRPLPPWDIRIKFQKASVSRCTLYWRDEGLVLLNRLRYRPSNSRLWNMVNV | |
| | TKAKGRHDLLDLKPFTEYEFQISSKLHLYKGSWSDWSESLRAQTPEEHPVGLLARVPLSLYSGHPVG | |
| | LLARVPLSLYSGLSGRSDNHGGGSGGGSIWELKKDVYVVELDWYPDAPGEMVVLTCDTPEEDGITWT | |
| | LDQSSEVLGSGKTLTIQVKEFGDAGQYTCHKGGEVLSHSLLLLHKKEDGIWSTDILKDQKEPKNKTE | |
| | LRCEAKNYSGRFTCWWLTTISTDLTFSVKSSRGSSDPQGVTCGAATLSAERVRGDNKEYEYSVECQE | |
| | DSACPAAEESLPIEVMVDAVHKLKYENYTSSFFIRDIIKPDPPKNLQLKPLKNSRQVEVSWEYPDTW | |
| | STPHSYFSLTFCVQVQGKSKREKKDRVFTDKTSATVICRKNASISVRAQDRYYSSSWSEWASVPCSH | |
| | PVGLLARVPLSLYSGHPVGLLARVPLSLYSGLSGRSDNHGGGSGGGSPKSSDKTHTCPPCPAPEAAG | |
| | GPSVFLFPPKPKDTLMISRTPEVTCVVVDVSHEDPEVKFNWYVDGVEVHNAKTKPREEQYNSTYRVV | |
| | SVLTVLHQDWLNGKEYKCKVSNKALGAPIEKTISKAKGQPREPRVYTLPPCRDELTKNQVSLTCLVK | |
| | GFYPSDIAVEWESNGQPENNYKTTPPVLVSDGSFTLYSKLTVDKSRWQQGNVESCSVMHEALHNHYT | |
| | QKSLSLSPGK | |
| SEQ ID | MEFGLSWVFLVALFRGVQCKIDACKRGDVTVKPSHVILLGSTVNITCSLKPRQGCFHYSRENKLILY | PRO081 |
| NO: | KFDRRINEHHGHSLNSQVTGLPLGTTLFVCKLACINSDEIQICGAEIFVGVAPEQPQNLSCIQKGEQ | |
| 663 | GTVACTWERGRDTHLYTEYTLQLSGPKNLTWQKQCKDIYCDYLDFGINLTPESPESNFTAKVTAVNS | |
| | LGSSSSLPSTFTFLDIVRPLPPWDIRIKFQKASVSRCTLYWRDEGLVLLNRLRYRPSNSRLWNMVNV | |
| | TKAKGRHDLLDLKPFTEYEFQISSKLHLYKGSWSDWSESLRAQTPEEHPVGLLARVPLSLYSGHPVG | |
| | LLARVPLSLYSGLSGRSDNHGGGSGGGSIWELKKDVYVVELDWYPDAPGEMVVLTCDTPEEDGITWT | |
| | LDQSSEVIGSGKTLTIQVKEFGDAGQYTCHKGGEVLSHSLLLLHKKEDGIWSTDILKDQKEPKNKTE | |
| | LRCEAKNYSGRFTCWWLTTISTDLTFSVKSSRGSSDPQGVTCGAATLSAERVRGDNKEYEYSVECQE | |
| | DSACPAAEESLPIEVMVDAVHKLKYENYTSSFFIRDIIKPDPPKNLQLKPLKNSRQVEVSWEYPDTW | |
| | STPHSYFSLTFCVQVQGKSKREKKDRVFTDKTSATVICRKNASISVRAQDRYYSSSWSEWASVPCSH | |
| | PVGLLARVPLSLYSGHPVGLLARVPLSLYSGLSGRSDNHGGGSGGGSPKSSDKTHTCPPCPAPEAAG | |
| | GPSVFLFPPKPKDTLMISRTPEVTCVVVDVSHEDPEVKFNWYVDGVEVHNAKTKPREEQYNSTYRVV | |
| | SVLTVLHQDWLNGKEYKCKVSNKALGAPIEKTISKAKGQPREPRVYTLPPCRDELTKNQVSLTCLVK | |
| | GFYPSDIAVEWESNGQPENNYKTTPPVLVSDGSFTLYSKLTVDKSRWQQGNVFSCSVMHEALHNHYT | |
| | QKSLSLSPGK | |

IL-2 amino acid sequence table

| SEQ ID | | |
|---|---|---|
| NO | Amino acid sequence | Notes |
| SEQ | MEFGLSWVELVALERGVQC | (SPH7- |
| ID | APTSSSTKKTQLQLEHLLLDLQMILNGINNYKNPKLTRMLTFKFYMPKKATELKHLQCLE | hIL2- |
| NO: | EELKPLEEVENLAQSKNFHLRPRDLISNINVIVLELKGSETTFMCEYADETATIVEFLNRWI | cleavable |
| 351 | TFCQSIISTLT | linkerL6- |
| | HPVGLLARVPLSLYSG HPVGLLARVPLSLYSG LSGRSDNH GGGSGGGS | Mask |
| | AVNGTSQFTCFYNSRANISCVWSQDGALQDTSCQVHAWPDRRRWNQTCELLPVSQASW | human |
| | ACNLILGAPDSQKLTTVDIVTLRVLCREGVRWRVMAIQDEKPFENLRLMAPISLQVVHVE | IL2Rbeta) |
| | THRCNISWEISQASHYFERHLEFEARTLSPGHTWEEAPLLTLKQKQEWICLETLTPDTQYE | |
| | FQVRVKPLQ | |
| SEQ | MEFGLSWVFLVALERGVQC | (SPH7- |
| ID | APTSSSTKKTQLQLEHLLLDLQMILNGINNYKNPKLTRMLTFKFYMPKKATELKHLQCLE | hIL2- |
| NO: | EELKPLEEVLNLAQSKNFHLRPRDLISNINVIVLELKGSETTFMCEYADETATIVEFLNRWI | cleavable |
| 352 | TFCQSIISTLT | linkerL6- |
| | HPVGLLARVPLSLYSG HPVGLLARVPLSLYSG LSGRSDNH | Mask |
| | GGGSGGGS | human |
| | AVNGTSQFTCFYNSRANISCVWSQDGALQDTSCQVHAWPDRRRWNQTCELLPVSQASW | IL2Rbeta- |
| | ACNLILGAPDSQKLTTVDIVTLRVLCREGVRWRVMAIQDFKPFENLRLMAPISLQVVHVE | linker- |
| | THRCNISWEISQASHYFERHLEFEARTLSPGHTWEEAPLLTLKQKQEWICLETLTPDTQYE | HSAbinding) |
| | FQVRVKPLQ | |
| | GGGGSGGGGSGGGGS | |
| | EVQLVESGGGLVQPGNSLRLSCAASGFTFSKFGMSWVRQAPGK | |
| | GLEWVSSISGSGRDTLYAESVKGRFTISRDNAKTTLYLQMNSLRPEDT | |
| | AVYYCTIGGSLSVSSQGTLVTVSS | |
| SEQ | MEFGLSWVFLVALFRGVQC | IL-2 |
| ID | APTSSSTKKTQLQLEHLLLDLQMILNGINNYKNPKLTRMLTFKFYMPKKATELKHLQCLE | (SPH7- |
| NO: | EELKPLEEVLNLAQSKNFHLRPRDLISNINVIVLELKGSETTFMCEYADETATIVEFLNRWI | hIL2- |
| 353 | TFCQSIISTLT | clinkerXi- |
| | GPPSGSSPMPYDLYHPSGGG | Mask |
| | AVNGTSQFTCFYNSRANISCVWSQDGALQDTSCQVHAWPDRRRWNQTCELLPVSQASW | human |
| | ACNLILGAPDSQKLTTVDIVTLRVLCREGVRWRVMAIQDFKPFENLRLMAPISLQVVHVE | IL2Rbeta) |
| | THRCNISWEISQASHYFERHLEFEARTLSPGHTWEEAPLLTLKQKQEWICLETLTPDTQYE | |
| SEQ | MEFGLSWVFLVALFRGVQC | IL-2 |
| ID | APTSSSTKKTQLQLEHLLLDLQMILNGINNYKNPKLTRMLTFKFYMPKKATELKHLQCLE | (SPH7- |
| NO: | EELKPLEEVLNLAQSKNFHLRPRDLISNINVIVLELKGSETTFMCEYADETATIVEFLNRWI | hIL2- |
| 354 | TFCQSIISTLT | linkerL6- |
| | HPVGLLARVPLSLYSG HPVGLLARVPLSLYSG LSGRSDNH | Mask_ |
| | GGGSGGGS | SCFab) |
| | EVQLVESGGGLVQPGGSLRLSCAASGFTFSSYTLAWVRQAPGKGLEWVAAIDSSSYTYSPDTVR | |
| | GRFTISRDNAKNSLYLQMNSLRAEDTAVYYCARDSNWDALDYWGQGTTVTVSSGGGGSGGGGSG | |
| | GGGSDIQMTQSPSSLSASVGDRVTITCKASQNVGTNVGWYQQKPGKAPKALIYSASFRYSGVPS | |
| | RFSGSGSGTDFTLTISSLQPEDFATYYCQQYYTYPYTFGGGTKVEIK | |
| SEQ | MEFGLSWVFLVALFRGVQC | IL-2 |
| ID | APTSSSTKKTQLQLEHLLLDLQMILNGINNYKNPKLTRMLTFKFYMPKKATELKHLQCLE | (SPH7- |
| NO: | EELKPLEEVLNLAQSKNFHLRPRDLISNINVIVLELKGSETTFMCEYADETATIVEFLNRWI | hIL2- |
| 355 | TFCQSIISTLT | linkerL6- |
| | HPVGLLARVPLSLYSG HPVGLLARVPLSLYSG LSGRSDNH GGGSGGGS | Mask_ |
| | EVQLVESGGGLVQPGGSLRLSCAASGFTFSSYTLAWVRQAPGKGLEWVAAIDSSSYTYSPDTVR | scFab- |
| | GRFTISRDNAKNSLYLQMNSLRAEDTAVYYCARDSNWDALDYWGQGTTVTVSSGGGGSGGGGSG | linker- |
| | GGGSDIQMTQSPSSLSASVGDRVTITCKASQNVGTNVGWYQQKPGKAPKALIYSASFRYSGVPS | HSAbinding) |
| | RFSGSGSGTDFTLTISSLQPEDFATYYCQQYYTYPYTFGGGTKVEIK | |
| | GGGGSGGGGSGGGGS | |
| | EVQLVESGGGLVQPGNSLRLSCAASGFTFSKFGMSWVRQAPGK | |
| | GLEWVSSISGSGRDTLYAESVKGRFTISRDNAKTTLYLQMNSLRPEDT | |
| | AVYYCTIGGSLSVSSQGTLVTVSS | |
| SEQ | MEFGLSWVFLVALFRGVQC | IL-2 |
| ID | APTSSSTKKTQLQLEHLLLDLQMILNGINNYKNPKLTRMLTFKFYMPKKATELKHLQCLE | (SPH7- |
| NO: | EELKPLEEVLNLAQSKNFHLRPRDLISNINVIVLELKGSETTFMCEYADETATIVEFLNRWI | hIL2- |
| 356 | TFCQSIISTLT | linkerL6- |
| | HPVGLLARVPLSLYSG HPVGLLARVPLSLYSG LSGRSDNH GGGSGGGS | Mask_ |
| | EVQLVESGGGLVQPGGSLRLSCAASGFTFSSYTLAWVRQAPGKGLEWVAAIDSSSYTYSPDTVR | scFab- |
| | GRFTISRDNAKNSLYLQMNSLRAEDTAVYYCARDSNWDALDYWGQGTTVTVSSGGGGSGGGGSG | linker- |
| | GGGSDIQMTQSPSSLSASVGDRVTITCKASQNVGTNVGWYQQKPGKAPKALIYSASFRYSGVPS | hIgG1- |
| | RFSGSGSGTDFTLTISSLQPEDFATYYCQQYYTYPYTFGGGTKVEIK | Fc) |
| | GGGGSGGGGSGGGGS | |
| | EPKSCDKTHTCPPCPAPEAAGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNKALGAPIEKTISKAKGQP | |
| | REPQVYTLPPSRDELTKNQVSLTCLVKGFYPSDIAVEWESNGQPENNYKTTPPVLDSDGSFFLY | |
| | SKLTVDKSRWQQGNVFSCSVMHEALHNHYTQKSLSLSPGK | |
| SEQ | MEFGLSWVFLVALFRGVQC | IL-2 |
| ID | APTSSSTKKTQLQLEHLLLDLQMILNGINNYKNPKLTRMLTFKFYMPKKATELKHLQCLE | (SPH7- |
| NO: | EELKPLEEVLNLAQSKNFHLRPRDLISNINVIVLELKGSETTFMCEYADETATIVEFLNRWI | hIL2- |
| 357 | TFCQSIISTLT | linkerL6- |
| | HPVGLLARVPLSLYSG HPVGLLARVPLSLYSG LSGRSDNH | Mask_ |
| | GGGSGGGS | scFab- |
| | EVQLVESGGGLVQPGGSLRLSCAASGFTFSSYTLAWVRQAPGKGLEWVAAIDSSSYTYSPDTVR | linker- |
| | GRFTISRDNAKNSLYLQMNSLRAEDTAVYYCARDSNWDALDYWGQGTTVTVSSGGGGSGGGGSG | mIgG2a- |
| | GGGSDIQMTQSPSSLSASVGDRVTITCKASQNVGTNVGWYQQKPGKAPKALIYSASFRYSGVPS | Fc) |
| | RFSGSGSGTDFTLTISSLQPEDFATYYCQQYYTYPYTFGGGTKVEIK | |
| | GGGGSGGGGSGGGGS | |
| | PRGPTIKPCPPCKCPAPNAAGG | |
| | PSVFIFPPKIKDVLMISLSPIVTCVVVDVSEDDPDVQISWFVNNVEVHTAQTQTHRED | |
| | YNSTLRVVSALPIQHQDWMSGKEFKCKVNNKDLGAPIERTIS | |
| | KPKGSVRA | |
| | PQVYVLPPPEEEMTKKQVTLTCMVTDFMPEDIYVEWTNNGKTELNYKNTEPVLdSDGSYfMYSK | |
| | LRVEKKNWVERNSYSCSVVHEGLHNHHTTKSFS | |
| | RTPGK | |
| SEQ | MEFGLSWVFLVALFRGVQC | IL-2 |
| ID | APTSSSTKKTQLQLEHLLLDLQMILNGINNYKNPKLTRMLTFKFYMPKKATELKHLQCLE | (SPH7- |
| NO: | EELKPLEEVLNLAQSKNFHLRPRDLISNINVIVLELKGSETTFMCEYADETATIVEFLNRWI | hIL2- |
| 358 | TFCQSIISTLT | cleavable |
| | HPVGLLARVPLSLYSG HPVGLLARVPLSLYSG LSGRSDNH | linkerL6- |
| | GGGSGGGS | Mask |
| | AVNGTSQFTCFYNSRANISCVWSQDGALQDTSCQVHAWPDRRRWNQTCELLPVSQASW | human |
| | ACNLILGAPDSQKLTTVDIVTLRVLCREGVRWRVMAIQDFKPFENLRLMAPISLQVVHVE | IL2Rbeta- |
| | THRCNISWEISQASHYFERHLEFEARTLSPGHTWEEAPLLTLKQKQEWICLETLTPDTQYE | linker- |
| | FQVRVKPLQ | hIgG1- |
| | GGGGSGGGGSGGGGS | Fc) |
| | EPKSCDKTHTCPPCPAPEAAGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNKALGAPIEKTISKAKGQP | |
| | REPQVYTLPPSRDELTKNQVSLTCLVKGFYPSDIAVEWESNGQPENNYKTTPPVLDSDGSFFLY | |
| | SKLTVDKSRWQQGNVFSCSVMHEALHNHYTQKSLSLSPGK | |
| SEQ | MEFGLSWVFLVALFRGVQC | IL-2 |
| ID | APTSSSTKKTQLQLEHLLLDLQMILNGINNYKNPKLTRMLTEKFYMPKKATELKHLQCLE | (SPH7- |
| NO: | EELKPLEEVLNLAQSKNFHLRPRDLISNINVIVLELKGSETTFMCEYADETATIVEFLNRWI | hIL2- |
| 359 | TFCQSIISTLT | linker- |
| | GGGGSGGGGSGGGGS | Mask_ |
| | EVQLVESGGGLVQPGGSLRLSCAASGFTFSSYTLAWVRQAPGKGLEWVAAIDSSSYTYSPDTVR | scFab- |
| | GRFTISRDNAKNSLYLQMNSLRAEDTAVYYCARDSNWDALDYWGQGTTVTVSSGGGGSGGGGSG | linker- |
| | GGGSDIQMTQSPSSLSASVGDRVTITCKASQNVGTNVGWYQQKPGKAPKALIYSASFRYSGVPS | HSAbinding) |
| | RFSGSGSGTDFTLTISSLQPEDFATYYCQQYYTYPYTFGGGTKVEIK | |
| | GGGGSGGGGSGGGGS | |
| | EVQLVESGGGLVQPGNSLRLSCAASGFTFSKFGMSWVRQAPGK | |
| | GLEWVSSISGSGRDTLYAESVKGRFTISRDNAKTTLYLQMNSLRPEDT | |
| | AVYYCTIGGSLSVSSQGTLVTVSS | |
| SEQ | MEFGLSWVFLVALFRGVQC | IL-2 |
| ID | APTSSSTKKTQLQLEHLLLDLQMILNGINNYKNPKLTRMLTFKFYMPKKATELKHLQCLE | (SPH7- |
| NO: | EELKPLEEVLNLAQSKNFHLRPRDLISNINVIVLELKGSETTFMCEYADETATIVEFLNRWI | hIL2- |
| 360 | TFCQSIISTLT | linker- |
| | GGGGSGGGGSGGGGS | Mask |
| | AVNGTSQFTCFYNSRANISCVWSQDGALQDTSCQVHAWPDRRRWNQTCELLPVSQASW | human |
| | ACNLILGAPDSQKLTTVDIVTLRVLCREGVRWRVMAIQDFKPFENLRLMAPISLQVVHVE | IL2Rbeta- |
| | THRCNISWEISQASHYFERHLEFEARTLSPGHTWEEAPLLTLKQKQEWICLETLTPDTQYE | linker- |
| | FQVRVKPLQ | HSAbinding) |
| | GGGGSGGGGSGGGGS | |
| | EVQLVESGGGLVQPGNSLRLSCAASGFTFSKFGMSWVRQAPGK | |
| | GLEWVSSISGSGRDTLYAESVKGRFTISRDNAKTTLYLQMNSLRPEDT | |
| | AVYYCTIGGSLSVSSQGTLVTVSS | |
| SEQ | MEFGLSWVFLVALFRGVQC | IL-2 |
| ID | APTSSSTKKTQLQLEHLLLDLQMILNGINNYKNPKLTRMLTFKFYMPKKATELKHLQCLE | (SPH7- |
| NO: | EELKPLEEVLNLAQSKNFHLRPRDLISNINVIVLELKGSETTFMCEYADETATIVEFLNRWI | hIL2- |
| 361 | TFCQSIISTLT | cleavable |
| | HPVGLLARVPLSLYSG HPVGLLARVPLSLYSG LSGRSDNH GGGSGGGS | linkerL6- |
| | AVNGTSQFTCFYNSRANISCVWSQDGALQDTSCQVHAWPDRRRWNQTCELLPVSQASW | Mask |
| | ACNLILGAPDSQKLTTVDIVTLRVLCREGVRWRVMAIQDFKPFENLRLMAPISLAVVHVE | human |
| | THRCNISWEISQASHYFERHLEFEARTLSPGHTWEEAPLLTLKQKQEWICLETLTPDTQYE | IL2Rbeta- |
| | FQVRVKPLQ | linker- |
| | GGGGGGGGSGGGGS | mIqG2a- |
| | PRGPTIKPCPPCKCPAPNAAGG | Fc) |
| | PSVFIFPPKIKDVLMISLSPIVTCVVVDVSEDDPDVQISWFVNNVEVHTAQTQTHRED | |
| | YNSTLRVVSALPIQHQDWMSGKEFKCKVNNKDLGAPIERTIS | |
| | KPKGSVRA | |
| | PQVYVLPPPEEEMTKKQVTLTCMVTDFMPEDIYVEWTNNGKTELNYKNTEPVLdSDGSYfMYSK | |
| | LRVEKKNWVERNSYSCSVVHEGLHNHHTTKSFS | |
| | RTPGK | |
| SEQ | MEFGLSWVFLVALFRGVQC | IL-2 |
| ID | APTSSSTKKTQLQLEHLLLDLQMILNGINNYKNPKLTRMLTFKFYMPKKATELKHLQCLE | (SPH7- |
| NO: | EELKPLEEVLNLAQSKNFHLRPRDLISNINVIVLELKGSETTFMCEYADETATIVEFLNRWI | hIL2) |
| 371 | TFCQSIISTLT | Aim: |
| | | unmasked |
| | | control |
| | | for in |
| | | vitro |
| | | masking |
| | | efficiency |
| | | validation |
| SEQ | MEFGLSWVFLVALERGVQC | IL-2 |
| ID | APTSSSTKKTQLQLEHLLLDLQMILNGINNYKNPKLTRMLTEKFYMPKKATELKHLQCLE | (SPH7- |
| NO: | EELKPLEEVLNLAQSKNFHLRPRDLISNINVIVLELKGSETTFMCEYADETATIVEFLNRWI | hIL2- |
| 372 | TFCQSIISTLT | linker- |
| | GGGGSGGGGSGGGGS | HSAbinding) |
| | EVQLVESGGGLVQPGNSLRLSCAASGFTFSKFGMSWVRQAPGK | Aim: |
| | GLEWVSSISGSGRDTLYAESVKGRFTISRDNAKTTLYLQMNSLRPEDT | unmasked |
| | AVYYCTIGGSLSVSSQGTLVTVSS | control |
| | | with half- |
| | | life |
| | | extension |
| | | testable |
| | | in |
| | | vivo |
| SEQ | MYRMQLLSCI ALSLALVTNS | Il-2 |
| ID | APTSSSTKKT QLQLEHLLLD LQMILNGINN YKNPKLTRML TFKFYMPKKA | secreted |
| NO: | TELKHLQCLE EELKPLEEVL NLAQSKNFHF DPRDVVSNIN VFVLELKGSE | version |
| 373 | TTFMCEYADE TATIVEFLNR WITFCQSIIS | with |
| | | mutation |
| SEQ | MYRMQLLSCIALSLALVTNSAPTSSSTKKTQLQLEHLLLDLQMILNGINNYKNPKLTRMLTFKF | |
| ID | YMPKKATELKHLQCLEEELKPLEEVLNLAQSKNFHFDPRDVVSNINVFVLELKGSETTFMCEYA | |
| NO: | DETATIVEFLNRWITFCQSIIS | |
| 481 | | |
| SEQ | MEFGLSWVFLVALFRGVQCAPTSSSTKKTQLQLEHLLLDLQMILNGINNYKNPKLTRMLTFKFY | |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLT | |
| 482 | | |
| SEQ | MEFGLSWVFLVALFRGVQCAPTSSSTKKTQLQLEHLLLDLQMILNGINNYKNPKLTRMLTFKFY | Pro054 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 614 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDFKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQ | |
| SEQ | MEFGLSWVFLVALFRGVQCAPTSSSTKKTQLQLEHLLLDLQMILNGINNYKNPKLTRMLTFKFY | Pro055 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 615 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDFKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQGGGGSGGGGSGGGGSEVQLVESGGGLVQPGNSLRLSCAASGFTFSKFGMSWVRQAPGKGL | |
| | EWVSSISGSGRDTLYAESVKGRFTISRDNAKTTLYLQMNSLRPEDTAVYYCTIGGSLSVSSQGT | |
| | LVTVSS | |
| SEQ | MEFGLSWVFLVALFRGVQCAPTSSSTKKTQLQLEHLLLDLQMILNGINNYKNPKLTRMLTFKFY | Pro056 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTGPPSGSSPMPYDLYHPSGGGAVNGTSQFTCFYNSRANISC | |
| 616 | VWSQDGALQDTSCQVHAWPDRRRWNQTCELLPVSQASWACNLILGAPDSQKLTTVDIVTLRVLC | |
| | REGVRWRVMAIQDFKPFENLRLMAPISLQVVHVETHRCNISWEISQASHYFERHLEFEARTLSP | |
| | GHTWEEAPLLTLKQKQEWICLETLTPDTQYE | |
| SEQ | MEFGLSWVFLVALFRGVQCAPTSSSTKKTQLQLEHLLLDLQMILNGINNYKNPKLTRMLTFKFY | pro061 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 617 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDFKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQGGGGSGGGGSGGGGSEPKSCDKTHTCPPCPAPEAAGGPSVFLFPPKPKDTLMISRTPEVT | |
| | CVVVDVSHEDPEVKFNWYVDGVEVHNAKTKPREEQYNSTYRVVSVLTVLHQDWLNGKEYKCKVS | |
| | NKALGAPIEKTISKAKGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYTQKSLSLSPGK | |
| SEQ | MEFGLSWVFLVALFRGVQCAPTSSSTKKTQLQLEHLLLDLQMILNGINNYKNPKLTRMLTFKFY | pro061 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 618 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDFKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQGGGGSGGGGSGGGGSEPKSCDKTHTCPPCPAPEAAGGPSVFLFPPKPKDTLMISRTPEVT | |
| | CVVVDVSHEDPEVKFNWYVDGVEVHNAKTKPREEQYNSTYRVVSVLTVLHQDWLNGKEYKCKVS | |
| | NKALGAPIEKTISKAKGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYTQKSLSLSPGK | |
| SEQ | MEFGLSWVFLVALFRGVQCAPTSSSTKKTQLQLEHLLLDLQMILNGINNYKNPKLTRMLTFKFY | pro064 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 619 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDFKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQGGGGSGGGGSGGGGSPRGPTIKPCPPCKCPAPNAAGGPSVFIFPPKIKDVLMISLSPIVT | |
| | CVVVDVSEDDPDVQISWFVNNVEVHTAQTQTHREDYNSTLRVVSALPIQHQDWMSGKEFKCKVN | |
| | NKDLGAPIERTISKPKGSVRAPQVYVLPPPEEEMTKKQVTLTCMVTDFMPEDIYVEWTNNGKTE | |
| | LNYKNTEPVLdSDGSYfMYSKLRVEKKNWVERNSYSCSVVHEGLHNHHTTKSFSRTPGK | |
| SEQ | MEFGLSWVFLVALFRGVQCAPTSSSTKKTQLQLEHLLLDLQMILNGINNYKNPKLTRMLTFKFY | pro064 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 620 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDFKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQGGGGSGGGGSGGGGSPRGPTIKPCPPCKCPAPNAAGGPSVFIFPPKIKDVLMISLSPIVT | |
| | CVVVDVSEDDPDVQISWFVNNVEVHTAQTQTHREDYNSTLRVVSALPIQHQDWMSGKEEKCKVN | |
| | NKDLGAPIERTISKPKGSVRAPQVYVLPPPEEEMTKKQVTLTCMVTDFMPEDIYVEWTNNGKTE | |
| | LNYKNTEPVLdSDGSYfMYSKLRVEKKNWVERNSYSCSVVHEGLHNHHTTKSFSRTPGK | |
| SEQ | MEFGLSWVFLVALFRGVQCAPTSSSTKKTQLQLEHLLLDLQMILNGINNYKNPKLTRMLTFKFY | pro065 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLT | |
| 621 | | |
| SEQ | METDTLLLWVLLLWVPGSTGAPTSSSTSSSTAEAQQQQQQQQQQQQHLEQLLMDLQELLSRMEN | pro108 |
| ID | YRNLKLPRMLTFKFYLPKQATELKDLQCLEDELGPLRHVLDLTQSKSFQLEDAENFISNIRVTV | |
| NO: | VKLKGSDNTFECQFDDESATVVDFLRRWIAFCQSIISTSPQHPVGLLARVPLSLYSGHPVGLLA | |
| 664 | RVPLSLYSGLSGRSDNHGGGSGGGSAVKNCSHLECFYNSRANVSCMWSHEEALNVTTCHVHAKS | |
| | NLRHWNKTCELTLVRQASWACNLILGSFPESQSLTSVDLLDINVVCWEEKGWRRVKTCDFHPED | |
| | NLRLVAPHSLQVLHIDTQRCNISWKVSQVSHYIEPYLEFEARRRLLGHSWEDASVLSLKQRQQW | |
| | LFLEMLIPSTSYEVQVRVKAQRNNTGTWSPWSQPLTFRTRPADPMKEGGGGSGGGGSGGGGSPR | |
| | GPTIKPCPPCKCPAPNAAGGPSVFIFPPKIKDVLMISLSPIVTCVVVDVSEDDPDVQISWFVNN | |
| | VEVHTAQTQTHREDYNSTLRVVSALPIQHQDWMSGKEFKCKVNNKDLGAPIERTISKPKGSVRA | |
| | PQVYVLPPPEEEMTKKQVTLTCMVTDFMPEDIYVEWTNNGKTELNYKNTEPVLdSDGSYfMYSK | |
| | LRVEKKNWVERNSYSCSVVHEGLHNHHTTKSFSRTPGK | |
| SEQ | METDTLLLWVLLLWVPGSTGAPTSSSTSSSTAEAQQQQQQQQQQQQHLEQLLMDLQELLSRMEN | pro109 |
| ID | YRNLKLPRMLTFKFYLPKQATELKDLQCLEDELGPLRHVLDLTQSKSFQLEDAENFISNIRVTV | |
| NO: | VKLKGSDNTFECQFDDESATVVDFLRRWIAFCQSIISTSPQHPVGLLARVPLSLYSGHPVGLLA | |
| 665 | RVPLSLYSGLSGRSDNHGGGSGGGSAVKNCSHLECFYNSRANVSCMWSHEEALNVTTCHVHAKS | |
| | NLRHWNKTCELTLVRQASWACNLILGSFPESQSLTSVDLLDINVVCWEEKGWRRVKTCDFHPED | |
| | NLRLVAPHSLQVLHIDTQRCNISWKVSQVSHYIEPYLEFEARRRLLGHSWEDASVLSLKQRQQW | |
| | LFLEMLIPSTSYEVQVRVKAQRGGGGSGGGGSGGGGSPRGPTIKPCPPCKCPAPNAAGGPSVFI | |
| | FPPKIKDVLMISLSPIVTCVVVDVSEDDPDVQISWFVNNVEVHTAQTQTHREDYNSTLRVVSAL | |
| | PIQHQDWMSGKEFKCKVNNKDLGAPIERTISKPKGSVRAPQVYVLPPPEEEMTKKQVTLTCMVT | |
| | DFMPEDIYVEWTNNGKTELNYKNTEPVLdSDGSYfMYSKLRVEKKNWVERNSYSCSVVHEGLHN | |
| | HHTTKSFSRTPGK | |
| SEQ | METDTLLLWVLLLWVPGSTGAPTSSSTSSSTAEAQQQQQQQQQQQQHLEQLLMDLQELLSRMEN | pro110 |
| ID | YRNLKLPRMLTFKFYLPKQATELKDLQCLEDELGPLRHVLDLTQSKSFQLEDAENFISNIRVTV | |
| NO: | VKLKGSDNTFECQFDDESATVVDFLRRWIAFCQSIISTSPQ | |
| 666 | | |
| SEQ | MEFGLSWVELVALFRGVQCAPTSSSTKKTQLQLEHLLLDLQMILNGINNYKNPKLTRMLTFKFY | prol11 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 667 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDEKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQGEFTTWSPWSQPLAFRTKPAALGKDTGGGGSGGGGSGGGGSEPKSCDKTHTCPPCPAPEA | |
| | AGGPSVFLFPPKPKDTLMISRTPEVTCVVVDVSHEDPEVKFNWYVDGVEVHNAKTKPREEQYNS | |
| | TYRVVSVLTVLHQDWLNGKEYKCKVSNKALGAPIEKTISKAKGQPREPQVYTLPPSRDELTKNQ | |
| | VSLTCLVKGFYPSDIAVEWESNGQPENNYKTTPPVLDSDGSFFLYSKLTVDKSRWQQGNVFSCS | |
| | VMHEALHNHYTQKSLSLSPGK | |

IL-15 amino acid sequence

| SEQ ID | | |
|---|---|---|
| NO | Amino acid sequence | Notes |
| SEQ | MASPQLRGYGVQAIPVLLLLLLLLLLPLRVTPGTTCPPPVSIEHADIRVKNYSVNSRERYVQNS | IL-15 |
| ID | GFKRKAGTSTLIECVINKNTNVAHWTTPSLKCIRDPSLAHYSPVPTVVTPKVTSQPESPSPSAK | and |
| NO: | EPEAFSPKSDTAMTTETAIMPGSRLTPSQTTSAGTTGTGSHKSSRAPSLAATMTLEPTASTSLR | sushi |
| 488 | ITEISPHSSKMTKVAISTSVLLVGAGVVMAFLAWYIKSRQPSQPCRVEVETMETVPMTVRASSK | |
| | EDEDTGAGGSGGSGGSGGSGGSGGSGGMKILKPYMRNTSISCYLCELLNSHFLTEAGIHVFILG | |
| | CVSVGLPKTEANWIDVRYDLEKIESLIQSIHIDTILYTDSDFHPSCKVTAMNCELLELQVILHE | |
| | YSNMTLNETVRNVLYLANSTLSSNKNVAESGCKECEELEEKTFTEFLQSFIRIVQMFINTS | |
| SEQ | MEFGLSWVELVALFRGVQCITCPPPMSVEHADIWVKSYSLYSRERYICNSGFKRKAGTSSLTEC | IL-15 |
| ID | VLNKATNVAHWTTPSLKCIRDPALVHQRPAPPGGSGGSGGSGGSGGSGGSGGNWVNVISDLKKI | and |
| NO: | EDLIQSMHIDATLYTESDVHPSCKVTAMKCFLLELQVISLESGDASIHDTVENLIILANNSLSS | sushi |
| 489 | NGNVTESGCKECEELEEKNIKEFLQSFVHIVQMFINTS | |
| SEQ | MSVLTQVLALLLLWLTGARCTTCPPPVSIEHADIRVKNYSVNSRERYVCNSGFKRKAGTSTLIE | PRO045 |
| ID | CVINKNTNVAHWTTPSLKCIRDPSLAGGSGGSGGSGGSGGSGGSGGNWIDVRYDLEKIESLIQS | |
| NO: | IHIDTTLYTDSDFHPSCKVTAMNCELLELQVILHEYSNMTLNETVRNVLYLANSTLSSNKNVAE | |
| 622 | SGCKECEELEEKTFTEFLQSFIRIVQMFINTS | |
| SEQ | MSVLTQVLALLLLWLTGARCEVQLVESGGGLVQPGNSLRLSCAASGFTFSKFGMSWVRQAPGKG | Pro101 |
| ID | LEWVSSISGSGRDTLYAESVKGRFTISRDNAKTTLYLQMNSLRPEDTAVYYCTIGGSLSVSSQG | |
| NO: | TLVTVSSHPVGLLARVPLSLYSGHPVGLLARVPLSLYSGLSGRSDNHGGGSGGGSTTCPPPVSI | |
| 623 | EHADIRVKNYSVNSRERYVCNSGEKRKAGTSTLIECVINKNTNVAHWTTPSLKCIRDPSLAGGS | |
| | GGSGGSGGSGGSGGSGGNWIDVRYDLEKIESLIQSIHIDTTLYTDSDFHPSCKVTAMNCFLLEL | |
| | QVILHEYSNMTLNETVRNVLYLANSTLSSNKNVAESGCKECEELEEKTFTEFLQSFIRIVQMFI | |
| | NTSHPVGLLARVPLSLYSGHPVGLLARVPLSLYSGLSGRSDNHGGGSGGGSEVQLVESGGGLVQ | |
| | PGNSLRLSCAASGFTFSKFGMSWVRQAPGKGLEWVSSISGSGRDTLYAESVKGRFTISRDNAKT | |
| | TLYLQMNSLRPEDTAVYYCTIGGSLSVSSQGTLVTVSS | |
| SEQ | MSVLTQVLALLLLWLTGARCTTCPPPVSIEHADIRVKNYSVNSRERYVCNSGFKRKAGTSTLIE | pro102 |
| ID | CVINKNTNVAHWTTPSLKCIRDPSLAGGSGGSGGSGGSGGSGGSGGNWIDVRYDLEKIESLIQS | |
| NO: | IHIDTILYTDSDFHPSCKVTAMNCELLELQVILHEYSNMTLNETVRNVLYLANSTLSSNKNVAE | |
| 624 | SGCKECEELEEKTFTEFLQSFIRIVQMFINTSHPVGLLARVPLSLYSGHPVGLLARVPLSLYSG | |
| | LSGRSDNHGGGSGGGSEVQLVESGGGLVQPGNSLRLSCAASGFTFSKFGMSWVRQAPGKGLEWV | |
| | SSISGSGRDTLYAESVKGRFTISRDNAKTTLYLQMNSLRPEDTAVYYCTIGGSLSVSSQGTLVT | |
| | VSS | |
| SEQ | MSVLTQVLALLLLWLTGARCEVQLVESGGGLVQPGNSLRLSCAASGFTFSKFGMSWVRQAPGKG | pro103 |
| ID | LEWVSSISGSGRDTLYAESVKGRFTISRDNAKTTLYLQMNSLRPEDTAVYYCTIGGSLSVSSQG | |
| NO: | TLVTVSSHPVGLLARVPLSLYSGHPVGLLARVPLSLYSGLSGRSDNHGGGSGGGSTTCPPPVSI | |
| 625 | EHADIRVKNYSVNSRERYVCNSGFKRKAGTSTLIECVINKNINVAHWTTPSLKCIRDPSLAGGS | |
| | GGSGGSGGSGGSGGSGGNWIDVRYDLEKIESLIQSIHIDTTLYTDSDFHPSCKVTAMNCFLLEL | |
| | QVILHEYSNMTLNETVRNVLYLANSTLSSNKNVAESGCKECEELEEKTFTEFLQSFIRIVQMFI | |
| | NTS | |
| SEQ | MSVLTQVLALLLLWLTGARCEVQLVESGGGLVQPGNSLRLSCAASGFTFSKFGMSWVRQAPGKG | pro104 |
| ID | LEWVSSISGSGRDTLYAESVKGRFTISRDNAKTTLYLQMNSLRPEDTAVYYCTIGGSLSVSSQG | |
| NO: | TLVTVSSGGGGSGGGGSGGGGSTTCPPPVSIEHADIRVKNYSVNSRERYVCNSGFKRKAGTSTL | |
| 626 | IECVINKNTNVAHWTTPSLKCIRDPSLAGGSGGSGGSGGSGGSGGSGGNWIDVRYDLEKIESLI | |
| | QSIHIDTTLYTDSDFHPSCKVTAMNCFLLELQVILHEYSNMTLNETVRNVLYLANSTLSSNKNV | |
| | AESGCKECEELEEKTFTEFLQSFIRIVQMFINTSGGGGSGGGGSGGGGSEVQLVESGGGLVQPG | |
| | NSLRISCAASGFTFSKFGMSWVRQAPGKGLEWVSSISGSGRDTLYAESVKGRFTISRDNAKTTL | |
| | YLQMNSLRPEDTAVYYCTIGGSLSVSSQGTLVTVSS | |
| SEQ | MEFGLSWVFLVALFRGVQCAPTSSSTKKTQLQLEHLLLDLQMILNGINNYKNPKLTRMLTFKFY | pro054 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 627 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDFKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQ | |
| SEQ | MEFGLSWVFLVALFRGVQCAPTSSSTKKTQLQLEHLLLDLQMILNGINNYKNPKLTRMLTFKFY | pro055 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 628 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDFKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQGGGGSGGGGSGGGGSEVQLVESGGGLVQPGNSLRLSCAASGFTFSKFGMSWVRQAPGKGL | |
| | EWVSSISGSGRDTLYAESVKGRFTISRDNAKTTLYLQMNSLRPEDTAVYYCTIGGSLSVSSQGT | |
| | LVTVSS | |
| SEQ | MEFGLSWVFLVALFRGVQCAPTSSSTKKTQLQLEHLLLDLQMILNGINNYKNPKLTRMLTFKFY | pro056 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTGPPSGSSPMPYDLYHPSGGGAVNGTSQFTCFYNSRANISC | |
| 629 | VWSQDGALQDTSCQVHAWPDRRRWNQTCELLPVSQASWACNLILGAPDSQKLTTVDIVTLRVLC | |
| | REGVRWRVMAIQDFKPFENLRLMAPISLQVVHVETHRCNISWEISQASHYFERHLEFEARTLSP | |
| | GHTWEEAPLLTLKQKQEWICLETLTPDTQYE | |
| SEQ | MEFGLSWVFLVALFRGVQCAPTSSSTKKTQLQLEHLLLDLQMILNGINNYKNPKLTRMLTFKFY | pro057 |
| ID | MPKKATELKHLQCLEEELKPLEEVINLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 630 | GGGSGGGSEVQLVESGGGLVQPGGSLRLSCAASGFTFSSYTLAWVRQAPGKGLEWVAAIDSSSY | |
| | TYSPDTVRGRFTISRDNAKNSLYLQMNSLRAEDTAVYYCARDSNWDALDYWGQGTTVTVSSGGG | |
| | GSGGGGSGGGGSDIQMTQSPSSLSASVGDRVTITCKASQNVGTNVGWYQQKPGKAPKALIYSAS | |
| | FRYSGVPSRFSGSGSGTDFTLTISSLQPEDFATYYCQQYYTYPYTFGGGTKVEIK | |
| SEQ | MEFGLSWVFLVALFRGVQCAPTSSSTKKTQLQLEHLLLDLQMILNGINNYKNPKLTRMLTFKFY | pro058 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 631 | GGGSGGGSEVQLVESGGGLVQPGGSLRLSCAASGFTFSSYTLAWVRQAPGKGLEWVAAIDSSSY | |
| | TYSPDTVRGRFTISRDNAKNSLYLQMNSLRAEDTAVYYCARDSNWDALDYWGQGTTVTVSSGGG | |
| | GSGGGGSGGGGSDIQMTQSPSSLSASVGDRVTITCKASQNVGTNVGWYQQKPGKAPKALIYSAS | |
| | FRYSGVPSRFSGSGSGTDFTLTISSLQPEDFATYYCQQYYTYPYTFGGGTKVEIKGGGGSGGGG | |
| | SGGGGSEVQLVESGGGLVQPGNSLRLSCAASGFTFSKFGMSWVRQAPGKGLEWVSSISGSGRDT | |
| | LYAESVKGRFTISRDNAKTTLYLQMNSLRPEDTAVYYCTIGGSLSVSSQGTLVTVSS | |
| SEQ | MEFGLSWVFLVALFRGVQCAPTSSSTKKTQLQLEHLLLDLQMILNGINNYKNPKLTRMLTFKFY | pro059 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 632 | GGGSGGGSEVQLVESGGGLVQPGGSLRLSCAASGFTFSSYTLAWVRQAPGKGLEWVAAIDSSSY | |
| | TYSPDTVRGRFTISRDNAKNSLYLQMNSLRAEDTAVYYCARDSNWDALDYWGQGTTVTVSSGGG | |
| | GSGGGGSGGGGSDIQMTQSPSSLSASVGDRVTITCKASQNVGTNVGWYQQKPGKAPKALIYSAS | |
| | FRYSGVPSRFSGSGSGTDFTLTISSLQPEDFATYYCQQYYTYPYTFGGGTKVEIKGGGGSGGGG | |
| | SGGGGSEPKSCDKTHTCPPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNGKEYKCKVSNKALGAPIEKTIS | |
| | KAKGQPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPGK | |
| SEQ | MEFGLSWVFLVALFRGVQCAPTSSSTKKTQLQLEHLLLDLQMILNGINNYKNPKLTRMLTFKFY | pro060 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 633 | GGGSGGGSEVQLVESGGGLVQPGGSLRLSCAASGFTFSSYTLAWVRQAPGKGLEWVAAIDSSSY | |
| | TYSPDTVRGRFTISRDNAKNSLYLQMNSLRAEDTAVYYCARDSNWDALDYWGQGTTVTVSSGGG | |
| | GSGGGGSGGGGSDIQMTQSPSSLSASVGDRVTITCKASQNVGTNVGWYQQKPGKAPKALIYSAS | |
| | FRYSGVPSRFSGSGSGTDFTLTISSLQPEDFATYYCQQYYTYPYTFGGGTKVEIKGGGGSGGGG | |
| | SGGGGSPRGPTIKPCPPCKCPAPNAAGGPSVFIFPPKIKDVLMISLSPIVTCVVVDVSEDDPDV | |
| | QISWFVNNVEVHTAQTQTHREDYNSTLRVVSALPIQHQDWMSGKEFKCKVNNKDLGAPIERTIS | |
| | KPKGSVRAPQVYVLPPPEEEMTKKQVTLTCMVTDFMPEDIYVEWTNNGKTELNYKNTEPVLdSD | |
| | GSYfMYSKLRVEKKNWVERNSYSCSVVHEGLHNHHTTKSFSRTPGK | |
| SEQ | MEFGLSWVFLVALFRGVQCAPTSSSTKKTQLQLEHLLLDLQMILNGINNYKNPKLTRMLTFKFY | pro061 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 634 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDFKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQGGGGSGGGGSGGGGSEPKSCDKTHTCPPCPAPEAAGGPSVFLFPPKPKDTLMISRTPEVT | |
| | CVVVDVSHEDPEVKFNWYVDGVEVHNAKTKPREEQYNSTYRVVSVLTVLHQDWLNGKEYKCKVS | |
| | NKALGAPIEKTISKAKGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYTQKSLSLSPGK | |
| SEQ | MEFGLSWVFLVALFRGVQCAPTSSSTKKTQLQLEHLLLDLQMILNGINNYKNPKLTRMLTFKFY | pro062 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTGGGGSGGGGSGGGGSEVQLVESGGGLVQPGGSLRLSCAAS | |
| 635 | GFTFSSYTLAWVRQAPGKGLEWVAAIDSSSYTYSPDTVRGRFTISRDNAKNSLYLQMNSLRAED | |
| | TAVYYCARDSNWDALDYWGQGTTVTVSSGGGGSGGGGSGGGGSDIQMTQSPSSLSASVGDRVTI | |
| | TCKASQNVGTNVGWYQQKPGKAPKALIYSASFRYSGVPSRFSGSGSGTDFTLTISSLQPEDFAT | |
| | YYCQQYYTYPYTFGGGTKVEIKGGGGSGGGGSGGGGSEVQLVESGGGLVQPGNSLRLSCAASGF | |
| | TFSKFGMSWVRQAPGKGLEWVSSISGSGRDTLYAESVKGRFTISRDNAKTTLYLQMNSLRPEDT | |
| | AVYYCTIGGSLSVSSQGTLVTVSS | |
| SEQ | MEFGLSWVFLVALFRGVQCAPTSSSTKKTQLQLEHLLLDLQMILNGINNYKNPKLTRMLTFKFY | pro063 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTGGGGSGGGGSGGGGSAVNGTSQFTCFYNSRANISCVWSQD | |
| 636 | GALQDTSCQVHAWPDRRRWNQTCELLPVSQASWACNLILGAPDSQKLTTVDIVTLRVLCREGVR | |
| | WRVMAIQDFKPFENLRLMAPISLQVVHVETHRCNISWEISQASHYFERHLEFEARTLSPGHTWE | |
| | EAPLLTLKQKQEWICLETLTPDTQYEFQVRVKPLQGGGGSGGGGSGGGGSEVQLVESGGGLVQP | |
| | GNSLRLSCAASGFTFSKFGMSWVRQAPGKGLEWVSSISGSGRDTLYAESVKGRFTISRDNAKTT | |
| | LYLQMNSLRPEDTAVYYCTIGGSLSVSSQGTLVTVSS | |
| SEQ | MEFGLSWVFLVALFRGVQCAPTSSSTKKTQLQLEHLLLDLQMILNGINNYKNPKLTRMLTFKFY | pro064 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTHPVGLLARVPLSLYSGHPVGLLARVPLSLYSGLSGRSDNH | |
| 637 | GGGSGGGSAVNGTSQFTCFYNSRANISCVWSQDGALQDTSCQVHAWPDRRRWNQTCELLPVSQA | |
| | SWACNLILGAPDSQKLTTVDIVTLRVLCREGVRWRVMAIQDFKPFENLRLMAPISLQVVHVETH | |
| | RCNISWEISQASHYFERHLEFEARTLSPGHTWEEAPLLTLKQKQEWICLETLTPDTQYEFQVRV | |
| | KPLQGGGGSGGGGSGGGGSPRGPTIKPCPPCKCPAPNAAGGPSVFIFPPKIKDVLMISLSPIVT | |
| | CVVVDVSEDDPDVQISWFVNNVEVHTAQTQTHREDYNSTLRVVSALPIQHQDWMSGKEFKCKVN | |
| | NKDLGAPIERTISKPKGSVRAPQVYVLPPPEEEMTKKQVTLTCMVTDFMPEDIYVEWTNNGKTE | |
| | LNYKNTEPVLASDGSYIMYSKLRVEKKNWVERNSYSCSVVHEGLHNHHTTKSFSRTPGK | |
| SEQ | MEFGLSWVFLVALFRGVQCAPTSSSTKKTQLQLEHLLLDLQMILNGINNYKNPKLTRMLTFKFY | pro065 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLT | |
| 638 | | |
| SEQ | MEFGLSWVFLVALFRGVQCAPTSSSTKKTQLQLEHLLLDLQMILNGINNYKNPKLTRMLTFKFY | pro066 |
| ID | MPKKATELKHLQCLEEELKPLEEVLNLAQSKNFHLRPRDLISNINVIVLELKGSETTFMCEYAD | |
| NO: | ETATIVEFLNRWITFCQSIISTLTGGGGSGGGGSGGGGSEVQLVESGGGLVQPGNSLRLSCAAS | |
| 639 | GFTFSKFGMSWVRQAPGKGLEWVSSISGSGRDTLYAESVKGRFTISRDNAKTTLYLQMNSLRPE | |
| | DTAVYYCTIGGSLSVSSQGTLVTVSS | |
| SEQ | METDTLLLWVLLLWVPGSTGRVIPVSGPARCLSQSRNLLKTTDDMVKTAREKLKHYSCTAEDID | PRO070 |
| ID | HEDITRDQTSTLKTCLPLELHKNESCLATRETSSTTRGSCLPPQKTSLMMTLCLGSIYEDLKMY | |
| NO: | QTEFQAINAALQNHNHQQIILDKGMLVAIDELMQSLNHNGETLRQKPPVGEADPYRVKMKLCIL | |
| 640 | LHAFSTRVVTINRVMGYLSSAHPVGLLARVPLSLYSGHPVGLLARVPLSLYSGLSGRSDNHGGG | |
| | SGGGSPRGPTIKPCPPCKCPAPNAAGGPSVFIFPPKIKDVLMISLSPIVTCVVVDVSEDDPDVQ | |
| | ISWFVNNVEVHTAQTQTHREDYNSTLRVVSALPIQHQDWMSGKEFKCKVNNKDLGAPIERTISK | |
| | PKGSVRAPQVYVLPPPEEEMTEKQVTLTCMVTDFMPEDIYVEWTNNGKTELNYKNTEPVLDSDG | |
| | SYFMYSWLRVEKKNWVERNSYSCSVVHEGLHNHHTTKSFSRTPG | |
| SEQ | METDTLLLWVLLLWVPGSTGMWELEKDVYVVEVDWTPDAPGETVNLTCDTPEEDDITWTSDQRH | PRO071 |
| ID | GVIGSGKTLTITVKEFLDAGQYTCHKGGETLSHSHLLLHKKENGIWSTEILKNFKNKTFLKCEA | |
| NO: | PNYSGRFTCSWLVQRNMDLKENIKSSSSSPDSRAVTCGMASLSAEKVTLDQRDYEKYSVSCQED | |
| 641 | VTCPTAEETLPIELALEARQQNKYENYSTSFFIRDIIKPDPPKNLQMKPLKNSQVEVSWEYPDS | |
| | WSTPHSYFSLKFFVRIQRKKEKMKETEEGCNQKGAFLVEKTSTEVQCKGGNVCVQAQDRYYNSS | |
| | CSKWACVPCRVRSHPVGLLARVPLSLYSGHPVGLLARVPLSLYSGLSGRSDNHGGGSGGGSPRG | |
| | PTIKPCPPCKCPAPNAAGGPSVFIFPPKIKDVIMISLSPIVTCVVVDVSEDDPDVQISWFVNNV | |
| | EVHTAQTQTHREDYNSTLRVVSALPIQHQDWMSGKEFKCKVNNKDLGAPIERTISKPKGSVRAP | |
| | RVYVLPPPEEEMTKKQVTLTCMVTDEMPEDIYVEWTNNGKTELNYKNTEPVLVSDGSYTMYSKL | |
| | RVEKKNWVERNSYSCSVVHEGLHNHHTTKSFSRTPG | |
| SEQ | METDTLLLWVLLLWVPGSTGRVIPVSGPARCLSQSRNLLKTTDDMVKTAREKLKHYSCTAEDID | PRO070 |
| ID | HEDITRDQTSTLKTCLPLELHKNESCLATRETSSTTRGSCLPPQKTSLMMTLCLGSIYEDLKMY | |
| NO: | QTEFQAINAALQNHNHQQIILDKGMLVAIDELMQSLNHNGETLRQKPPVGEADPYRVKMKLCIL | |
| 642 | LHAFSTRVVTINRVMGYLSSAHPVGLLARVPLSLYSGHPVGLLARVPLSLYSGLSGRSDNHGGG | |
| | SGGGSPRGPTIKPCPPCKCPAPNAAGGPSVFIFPPKIKDVLMISLSPIVTCVVVDVSEDDPDVQ | |
| | ISWEVNNVEVHTAQTQTHREDYNSTLRVVSALPIQHQDWMSGKEFKCKVNNKDLGAPIERTISK | |
| | PKGSVRAPQVYVLPPPEEEMTEKQVTLTCMVTDFMPEDIYVEWTNNGKTELNYKNTEPVLDSDG | |
| | SYFMYSWLRVEKKNWVERNSYSCSVVHEGLHNHHTTKSFSRTPG | |
| SEQ | METDTLLLWVLLLWVPGSTGQLGASGPGDGCCVEKTSFPEGASGSPLGPRNLSCYRVSKTDYEC | PRO072 |
| ID | SWQYDGPEDNVSHVLWCCFVPPNHTHTGQERCRYFSSGPDRTVQFWEQDGIPVLSKVNEWVESR | |
| NO: | LGNRTMKSQKISQYLYNWTKTTPPLGHIKVSQSHRQLRMDWNVSEEAGAEVQFRRRMPTTNWTL | |
| 643 | GDCGPQVNSGSGVLGDIRGSMSESCLCPSENMAQEIQIRRRRRLSSGAPGGPWSDWSMPVCVPP | |
| | EVLPQAHPVGLLARVPLSLYSGHPVGLLARVPLSLYSGLSGRSDNHGGGSGGGSRVIPVSGPAR | |
| | CLSQSRNLLKTTDDMVKTAREKLKHYSCTAEDIDHEDITRDQTSTLKTCLPLELHKNESCLATR | |
| | ETSSTTRGSCLPPQKTSLMMTLCLGSIYEDLKMYQTEFQAINAALQNHNHQQIILDKGMLVAID | |
| | ELMQSLNHNGETLRQKPPVGEADPYRVKMKLCILLHAFSTRVVTINRVMGYLSSAHPVGLLARV | |
| | PLSLYSGHPVGLLARVPLSLYSGLSGRSDNHGGGSGGGSPRGPTIKPCPPCKCPAPNAAGGPSV | |
| | FIFPPKIKDVLMISLSPIVTCVVVDVSEDDPDVQISWFVNNVEVHTAQTQTHREDYNSTLRVVS | |
| | ALPIQHQDWMSGKEFKCKVNNKDLGAPIERTISKPKGSVRAPQVYVLPPPEEEMTEKQVTLTCM | |
| | VTDFMPEDIYVEWTNNGKTELNYKNTEPVLDSDGSYFMYSWLRVEKKNWVERNSYSCSVVHEGL | |
| | HNHHTTKSFSRTPG | |
| SEQ | METDTLLLWVLLLWVPGSTGRVIPVSGPARCLSQSRNLLKTTDDMVKTAREKLKHYSCTAEDID | Pro074 |
| ID | HEDITRDQTSTLKTCLPLELHKNESCLATRETSSTTRGSCLPPQKTSLMMTLCLGSIYEDLKMY | |
| NO: | QTEFQAINAALQNHNHQQIILDKGMLVAIDELMQSLNHNGETLRQKPPVGEADPYRVKMKLCIL | |
| 644 | LHAFSTRVVTINRVMGYLSSAGGGGSGGGGSGGGGSPRGPTIKPCPPCKCPAPNAAGGPSVFIF | |
| | PPKIKDVLMISLSPIVTCVVVDVSEDDPDVQISWFVNNVEVHTAQTQTHREDYNSTLRVVSALP | |
| | IQHQDWMSGKEFKCKVNNKDIGAPIERTISKPKGSVRAPQVYVLPPPEEEMTEKQVTLTCMVTD | |
| | FMPEDIYVEWTNNGKTELNYKNTEPVLDSDGSYFMYSWLRVEKKNWVERNSYSCSVVHEGLHNH | |
| | HTTKSFSRTPG | |
| SEQ | METDTLLLWVLLLWVPGSTGRVIPVSGPARCLSQSRNLLKTTDDMVKTAREKLKHYSCTAEDID | Pro074 |
| ID | HEDITRDQTSTLKTCLPLELHKNESCLATRETSSTTRGSCLPPQKTSLMMTLCLGSIYEDLKMY | |
| NO: | QTEFQAINAALQNHNHQQIILDKGMLVAIDELMQSINHNGETLRQKPPVGEADPYRVKMKLCIL | |
| 645 | LHAFSTRVVTINRVMGYLSSAGGGGSGGGGSGGGGSPRGPTIKPCPPCKCPAPNAAGGPSVFIF | |
| | PPKIKDVLMISLSPIVTCVVVDVSEDDPDVQISWFVNNVEVHTAQTQTHREDYNSTLRVVSALP | |
| | IQHQDWMSGKEFKCKVNNKDLGAPIERTISKPKGSVRAPQVYVLPPPEEEMTEKQVTLTCMVTD | |
| | FMPEDIYVEWTNNGKTELNYKNTEPVLDSDGSYFMYSWLRVEKKNWVERNSYSCSVVHEGLHNH | |
| | HTTKSFSRTPG | |
| SEQ | METDTLLLWVLLLWVPGSTGQLGASGPGDGCCVEKTSFPEGASGSPLGPRNLSCYRVSKTDYEC | Pro076 |
| ID | SWQYDGPEDNVSHVLWCCFVPPNHTHTGQERCRYFSSGPDRTVQFWEQDGIPVLSKVNFWVESR | |
| NO: | LGNRTMKSQKISQYLYNWTKTTPPLGHIKVSQSHRQLRMDWNVSEEAGAEVQFRRRMPTTNWTL | |
| 646 | GDCGPQVNSGSGVLGDIRGSMSESCLCPSENMAQEIQIRRRRRLSSGAPGGPWSDWSMPVCVPP | |
| | EVLPQAGGGGSGGGGSGGGGSRVIPVSGPARCLSQSRNLLKTTDDMVKTAREKLKHYSCTAEDI | |
| | DHEDITRDQTSTLKTCLPLELHKNESCLATRETSSTTRGSCLPPQKTSLMMTLCLGSIYEDLKM | |
| | YQTEFQAINAALQNHNHQQIILDKGMLVAIDELMQSINHNGETLRQKPPVGEADPYRVKMKLCI | |
| | LLHAFSTRVVTINRVMGYLSSAGGGGSGGGGSGGGGSPRGPTIKPCPPCKCPAPNAAGGPSVFI | |
| | FPPKIKDVLMISLSPIVTCVVVDVSEDDPDVQISWFVNNVEVHTAQTQTHREDYNSTLRVVSAL | |
| | PIQHQDWMSGKEFKCKVNNKDLGAPIERTISKPKGSVRAPQVYVLPPPEEEMTEKQVTLTCMVT | |
| | DFMPEDIYVEWTNNGKTELNYKNTEPVLDSDGSYFMYSWLRVEKKNWVERNSYSCSVVHEGLHN | |
| | HHTTKSFSRTPG | |
| SEQ | METDTLLLWVLLLWVPGSTGQLGASGPGDGCCVEKTSFPEGASGSPLGPRNLSCYRVSKTDYEC | Pro076 |
| ID | SWQYDGPEDNVSHVLWCCFVPPNHTHTGQERCRYFSSGPDRTVQFWEQDGIPVLSKVNEWVESR | |
| NO: | LGNRTMKSQKISQYLYNWTKTTPPLGHIKVSQSHRQLRMDWNVSEEAGAEVQFRRRMPTTNWTL | |
| 647 | GDCGPQVNSGSGVLGDIRGSMSESCLCPSENMAQEIQIRRRRRLSSGAPGGPWSDWSMPVCVPP | |
| | EVLPQAGGGGSGGGGSGGGGSRVIPVSGPARCLSQSRNLLKTTDDMVKTAREKLKHYSCTAEDI | |
| | DHEDITRDQTSTLKTCLPLELHKNESCLATRETSSTTRGSCLPPQKTSLMMTLCLGSIYEDLKM | |
| | YQTEFQAINAALQNHNHQQIILDKGMLVAIDELMQSINHNGETLRQKPPVGEADPYRVKMKLCI | |
| | LLHAFSTRVVTINRVMGYLSSAGGGGSGGGGSGGGGSPRGPTIKPCPPCKCPAPNAAGGPSVFI | |
| | FPPKIKDVIMISLSPIVTCVVVDVSEDDPDVQISWFVNNVEVHTAQTQTHREDYNSTLRVVSAL | |
| | PIQHQDWMSGKEFKCKVNNKDLGAPIERTISKPKGSVRAPQVYVLPPPEEEMTEKQVTLTCMVT | |
| | DEMPEDIYVEWTNNGKTELNYKNTEPVLDSDGSYFMYSWLRVEKKNWVERNSYSCSVVHEGLHN | |
| | HHTTKSFSRTPG | |
| SEQ | MSVLTQVLALLLLWLTGARCNWIDVRYDLEKIESLIQSIHIDTTLYTDSDFHPSCKVTAMNCFL | pro046 |
| ID | LELQVILHEYSNMTLNETVRNVLYLANSTLSSNKNVAESGCKECEELEEKTFTEFLQSFIRIVQ | |
| NO: | MFINTSGGSGGSGGSGGSGGSGGSGGTTCPPPVSIEHADIRVKNYSVNSRERYVCNSGFKRKAG | |
| 668 | TSTLIECVINKNTNVAHWTTPSLKCIRDPSLA | |
| SEQ | MSVLTQVLALLLLWLTGARCNWIDVRYDLEKIESLIQSIHIDTTLYTDSDFHPSCKVTAMNCFL | pro047 |
| ID | LELQVILHEYSNMTLNETVRNVLYLANSTLSSNKNVAESGCKECEELEEKTFTEFLQSFIRIVQ | |
| NO: | MFINTSGGSGGSGGSGGSGGSGGSGGTTCPPPVSIEHADIRVKNYSVNSRERYVCNSGEKRKAG | |
| 669 | TSTLIECVINKNTNVAHWTTPSLKCIRDPSLAGGGGSGGGGSGGGGSGPTIKPCPPCKCPAPNA | |
| | AGGPSVFIFPPKIKDVIMISLSPIVTCVVVDVSEDDPDVQISWFVNNVEVHTAQTQTHREDYNS | |
| | TLRVVSALPIQHQDWMSGKEFKCKVNNKDLGAPIERTISKPKGSVRAPQVYVLPPPEEEMTKKQ | |
| | VTLTCMVTDFMPEDIYVEWTNNGKTELNYKNTEPVLDSDGSYFMYSKLRVEKKNWVERNSYSCS | |
| | VVHEGLHNHHTTKSFSRTPG | |

Interferon alpha (IFN-α) amino acid sequence

| SEQ ID | | |
|---|---|---|
| NO | Amino acid sequence | Notes |
| SEQ ID | MEFGLSWVFLVALFRGVQCCDLPQTHSLGSRRTLMLLAQMRRISLFSCLKDRHDFGFPQEEFGNQFQKA | |
| NO: | ETIPVLHEMIQQIFNLFSTKDSSAAWDETLLDKFYTELYQQLNDLEACVIQGVGVTETPLMKEDSILAV | |
| 490 | RKYFQRITLYLKEKKYSPCAWEVVRAEIMRSFSLSTNLQESLRSKE | |
| SEQ ID | MEFGLSWVELVALFRGVQCEVQLVESGGGLVQPGNSLRLSCAASGFTFSKFGMSWVRQAPGKGLEWVSS | pro089 |
| NO: | ISGSGRDTLYAESVKGRFTISRDNAKTTLYLQMNSLRPEDTAVYYCTIGGSLSVSSQGTLVTVSSHPVG | |
| 601 | LLARVPLSLYSGHPVGLLARVPLSLYSGLSGRSDNHGGGSGGGSCDLPQTHNLRNKRALTLLVQMRRLS | |
| | PLSCLKDRKDEGFPQEKVDAQQIKKAQAIPVISELTQQILNIFTSKDSSAAWNTTLLDSFCNDLHQQLN | |
| | DLQGCLMQQVGVQEFPLTQEDALLAVRKYFHRITVYLREKKHSPCAWEVVRAEVWRALSSSANVIGRLR | |
| | EEKHPVGLLARVPLSLYSGHPVGLLARVPLSLYSGLSGRSDNHGGGSGGGSEVQLVESGGGLVQPGNSL | |
| | RLSCAASGFTFSKFGMSWVRQAPGKGLEWVSSISGSGRDTLYAESVKGRFTISRDNAKTTLYLQMNSLR | |
| | PEDTAVYYCTIGGSLSVSSQGTLVTVSS | |
| SEQ ID | MEFGLSWVFLVALFRGVQCEVQLVESGGGLVQPGNSLRLSCAASGFTFSKFGMSWVRQAPGKGLEWVSS | pro090 |
| NO: | ISGSGRDTLYAESVKGRFTISRDNAKTTLYLQMNSLRPEDTAVYYCTIGGSLSVSSQGTLVTVSSHPVG | |
| 602 | LLARVPLSLYSGHPVGLLARVPLSLYSGLSGRSDNHGGGSGGGSCDLPQTHNLRNKRALTLLVQMRRLS | |
| | PLSCLKDRKDFGFPQEKVDAQQIKKAQAIPVLSELTQQILNIFTSKDSSAAWNTTLLDSFCNDLHQQLN | |
| | DLQGCLMQQVGVQEFPLTQEDALLAVRKYFHRITVYLREKKHSPCAWEVVRAEVWRALSSSANVLGRLR | |
| | EEK | |
| SEQ ID | MEFGLSWVFLVALFRGVQCCDLPQTHNLRNKRALTLLVQMRRLSPLSCLKDRKDFGFPQEKVDAQQIKK | pro091 |
| NO: | AQAIPVISELTQQILNIFTSKDSSAAWNTTLLDSFCNDLHQQLNDLQGCLMQQVGVQEFPLTQEDALLA | |
| 603 | VRKYFHRITVYLREKKHSPCAWEVVRAEVWRALSSSANVLGRLREEKHPVGLLARVPLSLYSGHPVGLL | |
| | ARVPLSLYSGLSGRSDNHGGGSGGGSEVQLVESGGGLVQPGNSLRLSCAASGFTFSKFGMSWVRQAPGK | |
| | GLEWVSSISGSGRDTLYAESVKGRFTISRDNAKTTLYLQMNSLRPEDTAVYYCTIGGSLSVSSQGTLVT | |
| | VSS | |
| SEQ ID | MEFGLSWVFLVALFRGVQCENLKPPENIDVYIIDDNYTLKWSSHGESMGSVTFSAEYRTKDEAKWLKVP | pro092 |
| NO: | ECQHTTTTKCEFSLLDTNVYIKTQFRVRAEEGNSTSSWNEVDPFIPFYTAHMSPPEVRLEAEDKAILVH | |
| 604 | ISPPGQDGNMWALEKPSFSYTIRIWQKSSSDKKTINSTYYVEKIPELLPETTYCLEVKAIHPSLKKHSN | |
| | YSTVQCISTTVANKMPVPGNLQVDAQGKSYVLKWDYIASADVLFRAQWLPGYSKSSSGSRSDKWKPIPT | |
| | CANVQTTHCVFSQDTVYTGTFFLHVQASEGNHTSFWSEEKFIDSQKHGGGGSSGGPALFKSSFPPGSCD | |
| | LPQTHNLRNKRALTLLVQMRRLSPLSCLKDRKDFGFPQEKVDAQQIKKAQAIPVLSELTQQILNIFTSK | |
| | DSSAAWNTTLLDSFCNDLHQQLNDLQGCLMQQVGVQEFPLTQEDALLAVRKYFHRITVYLREKKHSPCA | |
| | WEVVRAEVWRALSSSANVLGRLREEKSGGPALFKSSFPPGSEVQLVESGGGLVQPGNSLRISCAASGET | |
| | FSKFGMSWVRQAPGKGLEWVSSISGSGRDTLYAESVKGRFTISRDNAKTTLYLQMNSLRPEDTAVYYCT | |
| | IGGSLSVSSQGTLVTVSS | |
| SEQ ID | MEFGLSWVFLVALFRGVQCENLKPPENIDVYIIDDNYTLKWSSHGESMGSVTFSAEYRTKDEAKWLKVP | pro093 |
| NO: | ECQHTTTTKCEFSLLDTNVYIKTQFRVRAEEGNSTSSWNEVDPFIPFYTAHMSPPEVRLEAEDKAILVH | |
| 605 | ISPPGQDGNMWALEKPSFSYTIRIWQKSSSDKKTINSTYYVEKIPELLPETTYCLEVKAIHPSLKKHSN | |
| | YSTVQCISTTVANKMPVPGNLQVDAQGKSYVLKWDYIASADVLFRAQWLPGYSKSSSGSRSDKWKPIPT | |
| | CANVQTTHCVESQDTVYTGTFFLHVQASEGNHTSEWSEEKFIDSQKHHPVGLLARVPLSLYSGHPVGLL | |
| | ARVPLSLYSGLSGRSDNHGGGSGGGSCDLPQTHNLRNKRALTLLVQMRRLSPLSCLKDRKDEGFPQEKV | |
| | DAQQIKKAQAIPVLSELTQQILNIFTSKDSSAAWNTTLLDSFCNDLHQQLNDLQGCLMQQVGVQEFPLT | |
| | QEDALLAVRKYFHRITVYLREKKHSPCAWEVVRAEVWRALSSSANVLGRLREEKHPVGLLARVPLSLYS | |
| | GHPVGLLARVPLSLYSGLSGRSDNHGGGSGGGSEVQLVESGGGLVQPGNSLRLSCAASGFTFSKFGMSW | |
| | VRQAPGKGLEWVSSISGSGRDTLYAESVKGRFTISRDNAKTTLYLQMNSLRPEDTAVYYCTIGGSLSVS | |
| | SQGTLVTVSS | |
| SEQ ID | MEFGLSWVFLVALFRGVQCCDLPQTHNLRNKRALTLLVQMRRLSPLSCLKDRKDFGFPQEKVDAQQIKK | pro094 |
| NO: | AQAIPVISELTQQILNIFTSKDSSAAWNTTLLDSFCNDLHQQLNDLQGCLMQQVGVQEFPLTQEDALLA | |
| 606 | VRKYFHRITVYLREKKHSPCAWEVVRAEVWRALSSSANVLGRLREEK | |
| SEQ ID | MEFGLSWVFLVALFRGVQCEVQLVESGGGLVQPGNSLRLSCAASGFTFSKFGMSWVRQAPGKGLEWVSS | pro095 |
| NO: | ISGSGRDTLYAESVKGRFTISRDNAKTTLYLQMNSLRPEDTAVYYCTIGGSLSVSSQGTLVTVSSGGGG | |
| 607 | SGGGGSGGGGSCDLPQTHNLRNKRALTLLVQMRRLSPLSCLKDRKDFGFPQEKVDAQQIKKAQAIPVLS | |
| | ELTQQILNIFTSKDSSAAWNTTLLDSFCNDLHQQLNDLQGCLMQQVGVQEFPLTQEDALLAVRKYFHRI | |
| | TVYEREKKHSPCAWEVVRAEVWRALSSSANVLGRLREEKGGGGSGGGGSGGGGSEVQLVESGGGLVQPG | |
| | NSLRLSCAASGFTFSKFGMSWVRQAPGKGLEWVSSISGSGRDTLYAESVKGRFTISRDNAKTTLYLQMN | |
| | SLRPEDTAVYYCTIGGSLSVSSQGTLVTVSS | |
| SEQ ID | MEFGLSWVFLVALFRGVQCEVQLVESGGGLVQPGNSLRLSCAASGFTFSKFGMSWVRQAPGKGLEWVSS | pro082 |
| NO: | ISGSGRDTLYAESVKGRFTISRDNAKTTLYLQMNSLRPEDTAVYYCTIGGSLSVSSQGTLVTVSSHPVG | |
| 608 | LLARVPLSLYSGHPVGLLARVPLSLYSGLSGRSDNHGGGSGGGSCDLPQTHSLGSRRTLMLLAQMRRIS | |
| | LFSCLKDRHDFGFPQEEFGNQFQKAETIPVLHEMIQQIFNLFSTKDSSAAWDETLLDKFYTELYQQLND | |
| | LEACVIQGVGVTETPLMKEDSILAVRKYFQRITLYLKEKKYSPCAWEVVRAEIMRSFSLSTNLQESLRS | |
| | KEHPVGLLARVPLSLYSGHPVGLLARVPLSLYSGLSGRSDNHGGGSGGGSEVQLVESGGGLVQPGNSLR | |
| | LSCAASGETFSKFGMSWVRQAPGKGLEWVSSISGSGRDTLYAESVKGRFTISRDNAKTTLYLQMNSLRP | |
| | EDTAVYYCTIGGSLSVSSQGTLVTVSS | |
| SEQ ID | MEFGLSWVFLVALFRGVQCISYDSPDYTDESCTFKISLRNERSILSWELKNHSIVPTHYTLLYTIMSKP | pro083 |
| NO: | EDLKVVKNCANTTRSFCDLTDEWRSTHEAYVTVLEGESGNTTLFSCSHNFWLAIDMSFEPPEFEIVGET | |
| 609 | NHINVMVKFPSIVEEELQFDLSLVIEEQSEGIVKKHKPEIKGNMSGNETYIIDKLIPNTNYCVSVYLEH | |
| | SDEQAVIKSPLKCTLLPPGQESESAESAKSGGPALFKSSFPPGSCDLPQTHSLGSRRTLMLLAQMRRIS | |
| | LFSCLKDRHDFGFPQEEFGNQFQKAETIPVLHEMIQQIFNLESTKDSSAAWDETLLDKFYTELYQQLND | |
| | LEACVIQGVGVTETPLMKEDSILAVRKYFQRITLYLKEKKYSPCAWEVVRAEIMRSFSLSTNLQESLRS | |
| | KESGGPALFKSSFPPGSEVQLVESGGGLVQPGNSLRLSCAASGFTFSKFGMSWVRQAPGKGLEWVSSIS | |
| | GSGRDTLYAESVKGRETISRDNAKTTLYLQMNSLRPEDTAVYYCTIGGSLSVSSQGTLVTVSS | |
| SEQ ID | MEFGLSWVFLVALFRGVQCISYDSPDYTDESCTFKISLRNERSILSWELKNHSIVPTHYTLLYTIMSKP | pro084 |
| NO: | EDLKVVKNCANTTRSFCDLTDEWRSTHEAYVTVLEGESGNTTLESCSHNEWLAIDMSFEPPEFEIVGET | |
| 610 | NHINVMVKFPSIVEEELQFDLSLVIEEQSEGIVKKHKPEIKGNMSGNFTYIIDKLIPNTNYCVSVYLEH | |
| | SDEQAVIKSPLKCTLLPPGQESESAESAKHPVGLLARVPLSLYSGHPVGLLARVPLSLYSGLSGRSDNH | |
| | GGGSGGGSCDLPQTHSLGSRRTLMLLAQMRRISLESCLKDRHDFGFPQEEFGNQFQKAETIPVLHEMIQ | |
| | QIFNLFSTKDSSAAWDETLLDKFYTELYQQLNDLEACVIQGVGVTETPLMKEDSILAVRKYFQRITLYL | |
| | KEKKYSPCAWEVVRAEIMRSFSLSTNLQESLRSKEHPVGLLARVPLSLYSGHPVGLLARVPLSLYSGLS | |
| | GRSDNHGGGSGGGSEVQLVESGGGLVQPGNSLRLSCAASGFTFSKFGMSWVRQAPGKGLEWVSSISGSG | |
| | RDTLYAESVKGRFTISRDNAKTTLYLQMNSLRPEDTAVYYCTIGGSLSVSSQGTLVTVSS | |
| SEQ ID | MEFGLSWVFLVALFRGVQCEVQLVESGGGLVQPGNSLRLSCAASGFTFSKFGMSWVRQAPGKGLEWVSS | pro085 |
| NO: | ISGSGRDTLYAESVKGRFTISRDNAKTTLYLQMNSLRPEDTAVYYCTIGGSLSVSSQGTLVTVSSHPVG | |
| 611 | LLARVPLSLYSGHPVGLLARVPLSLYSGLSGRSDNHGGGSGGGSCDLPQTHSLGSRRTLMLLAQMRRIS | |
| | LFSCLKDRHDFGFPQEEFGNQFQKAETIPVLHEMIQQIFNLFSTKDSSAAWDETLLDKFYTELYQQLND | |
| | LEACVIQGVGVTETPLMKEDSILAVRKYFQRITLYLKEKKYSPCAWEVVRAEIMRSFSLSTNLQESLRS | |
| | KE | |
| SEQ ID | MEFGLSWVFLVALFRGVQCCDLPQTHSLGSRRTLMLLAQMRRISLESCLKDRHDFGFPQEEFGNQFQKA | pro086 |
| NO: | ETIPVLHEMIQQIFNLFSTKDSSAAWDETLLDKFYTELYQQLNDLEACVIQGVGVTETPLMKEDSILAV | |
| 612 | RKYFQRITLYLKEKKYSPCAWEVVRAEIMRSFSLSTNLQESLRSKEHPVGLLARVPLSLYSGHPVGLLA | |
| | RVPLSLYSGLSGRSDNHGGGSGGGSEVQLVESGGGLVQPGNSLRLSCAASGFTFSKEGMSWVRQAPGKG | |
| | LEWVSSISGSGRDTLYAESVKGRFTISRDNAKTTLYLQMNSLRPEDTAVYYCTIGGSLSVSSQGTLVTV | |
| | SS | |
| SEQ ID | MEFGLSWVELVALFRGVQCCDLPQTHSLGSRRTLMLLAQMRRISLFSCLKDRHDFGFPQEEFGNQFQKA | pro088 |
| NO: | ETIPVLHEMIQQIFNLFSTKDSSAAWDETLLDKFYTELYQQLNDLEACVIQGVGVTETPLMKEDSILAV | |
| 613 | RKYFQRITLYLKEKKYSPCAWEVVRAEIMRSFSLSTNLQESLRSKE | |
| SEQ ID | MEFGLSWVFLVALFRGVQCEVQLVESGGGLVQPGNSLRLSCAASGFTFSKFGMSWVRQAPGKGLEWVSS | pro087 |
| NO: | ISGSGRDTLYAESVKGRFTISRDNAKTTLYLQMNSLRPEDTAVYYCTIGGSLSVSSQGTLVTVSSGGGG | |
| 674 | SGGGGSGGGGSCDLPQTHSLGSRRTLMLLAQMRRISLFSCLKDRHDFGFPQEEFGNQFQKAETIPVLHE | |
| | MIQQIFNLFSTKDSSAAWDETLLDKFYTELYQQLNDLEACVIQGVGVTETPLMKEDSILAVRKYFQRIT | |
| | LYLKEKKYSPCAWEVVRAEIMRSFSLSTNLQESLRSKEGGGGSGGGGSGGGGSEVQLVESGGGLVQPGN | |
| | SLRLSCAASGFTFSKFGMSWVRQAPGKGLEWVSSISGSGRDTLYAESVKGRETISRDNAKTTLYLQMNS | |
| | LRPEDTAVYYCTIGGSLSVSSQGTLVTVSS | |
| SEQ ID | MEFGLSWVFLVALFRGVQCEVQLVESGGGLVQPGNSLRLSCAASGFTFSKFGMSWVRQAPGKGLEWVSS | pro100 |
| NO: | ISGSGRDTLYAESVKGRFTISRDNAKTTLYLQMNSLRPEDTAVYYCTIGGSLSVSSQGTLVTVSSGGGG | |
| 678 | SGGGGSGGGGSITCPPPMSVEHADIWVKSYSLYSRERYICNSGFKRKAGTSSLTECVLNKATNVAHWTT | |
| | PSLKCIRDPALVHQRPAPPGGSGGSGGSGGSGGSGGSGGNWVNVISDLKKIEDLIQSMHIDATLYTESD | |
| | VHPSCKVTAMKCFLLELQVISLESGDASIHDTVENLIILANNSLSSNGNVTESGCKECEELEEKNIKEF | |
| | LQSFVHIVQMFINTSGGGGSGGGGSGGGGSEVQLVESGGGLVQPGNSLRLSCAASGFTESKFGMSWVRQ | |
| | APGKGLEWVSSISGSGRDTLYAESVKGRFTISRDNAKTTLYLQMNSLRPEDTAVYYCTIGGSLSVSSQG | |
| | TLVTVSS | |
| SEQ ID | MEFGLSWVFLVALFRGVQCEVQLVESGGGLVQPGNSLRLSCAASGFTFSKFGMSWVRQAPGKGLEWVSS | pro098 |
| NO: | ISGSGRDTLYAESVKGRFTISRDNAKTTLYLQMNSLRPEDTAVYYCTIGGSLSVSSQGTLVTVSSHPVG | |
| 685 | LLARVPLSLYSGHPVGLLARVPLSLYSGLSGRSDNHGGGSGGGSITCPPPMSVEHADIWVKSYSLYSRE | |
| | RYICNSGFKRKAGTSSLTECVLNKATNVAHWTTPSLKCIRDPALVHQRPAPPGGSGGSGGSGGSGGSGG | |
| | SGGNWVNVISDLKKIEDLIQSMHIDATLYTESDVHPSCKVTAMKCFLLELQVISLESGDASIHDTVENL | |
| | IILANNSLSSNGNVTESGCKECEELEEKNIKEFLQSFVHIVQMFINTS | |

Oncoselective nucleic acid sequence

| SEQ ID | | |
|---|---|---|
| NO | Nucleic acid sequence | Notes |
| 1 | GUUUGUUUACUUGGCGAGACUUGGAGCUGAGGUCAUUUGGAGC | H2BC1 |
| | UGUUUAAUACUGAAGAGCUGUUGAGCACUGGAAAGUGCUGUGU | 5′ |
| | AACCCUGGAAAAGAACCGUGUAACGCUGCAGAAGUGUGUGGUAG | (5UTR017) |
| | CU | |
| 2 | AGAGCCACGCGGCACGCCCGGGAGGCUUUCUCUGGCUGGUAACC | H2BC1 |
| | GCUACUCCCGGACACCAGACCACCGCCUUCCGUACACAGGGGCCC | 5′ |
| | GCAUCCCACCCUCCCGGACCUAAGAGCCUGGGUCCCCUGUUUCCG | (5UTR018) |
| | GAGGUCCGCUUCCCGGCCCCCAGAUUCUGGCAUCCCAGCCCUCAG | |
| | UGUCCAAGACCCAGGCAGCCCGGGUCCCCGCCUCCCGGAUCCAGG | |
| | CGUCCGGGAUCUGCGCCACCAGAACCUAGCCUCCUGCAGACCUCC | |
| | GCCAUCUGGGGGCACUCAACCUCCUGGAGCCAAGGGCCCCACGU | |
| | CCCACCCAGAGAAACUCUCGUAUUCCCAGCUCCUAGGGCCAAGG | |
| | AACCCGGGCGCUCCGAACUCCCAGCUUUCGGACAUCUGGCACAC | |
| | GGGGCAGAGCAGAGAAGCCUCAGCGCCCAGCCUGGGGAAUUUAA | |
| | ACACUCCAGCUUCCAAGAGCCAAGGAACUUCAGUGCUGUGAACU | |
| | CACAACUCUAAGGAGCCCUCCAAAGUUCCAGUCUCCAGGUGCUG | |
| | UUACUCAACUCAGUCCUAGGAACGUCGGGUCCUGGGAAGGAGCC | |
| | CAAGCGCUCCCAGCCAGCUUCCAGGCGCUAAGAAACCCCGGUGC | |
| | UUCCCAUC | |
| 3 | AGUAGCAGCAGCGCCGGGUCCCGUGCGGAGGUGCUCCUCGCAGA | HNRNPA2B1 |
| | GUUGUUUCUCGAGCAGCGGCAGUUCUCACUACAGCGCCAGGACG | 5′ UTR |
| | AGUCCGGUUCGUGUUCGUCCGCGGAGAUCUCUCUCAUCUCGCUC | (5UTR010) |
| | GGCUGCGGGAAAUCGGGCUGAAGCGACUGAGUCCGCG | |
| 4 | GGACACAUGGUUACCACCUGGCCCUGAGUGCAGUCAGACUGGGA | Human |
| | CAAAGGAGCCGUGAAACACGCAAGGAGCUUCUGGCUUCUCAGCU | CD320 |
| | UUGAGACCUGGGCUCUUUGGAGCACAGAGAACUUUGAUUUUUAA | 3′ UTR |
| | GCUUCUACAGAUAUGGUCCUAGAGACUGGGGUCCUCAGACACUC | SEQ ID |
| | CCGCUGUAUGUGGUGAGCUUGGCUGGAAACUUGCCAUAGCCAGG | NO: 4 |
| | ACUGAGGGGCUGGCCCCAGGACGCUCUCAAGAAAUGGGGCUACU | (3UTR152) |
| | GUGGUUUAGACACUCCUGCUGCUCACAUUGAUGGGUGGCUAUUA | |
| | AAG | |
| 5 | GGACAAGCACUUGCCACCACCGUCACUCAGCCCUGGGCGUAGCC | Mouse |
| | GGACAGGAGGAGAGCAGUGAUGCGGAUGGGUACCCGGGCACACC | CD320 |
| | AGCCCUCAGAGACCUGAGCUCUUCUGGCCACGUGGAACCUCGAA | 3′ UTR |
| | CCCGAGCUCCUGCAGAAGUGGCCCUGGAGAUUGAGGGUCCCUGG | SEQ ID |
| | ACACUCCCUAUGGAGAUCCGGGGAGCUAGGAUGGGGAACCUGCC | NO: 5 |
| | ACAGCCAGAACUGAGGGGCUGGCCCCAGGCAGCUCCCAGGGGGU | (3UTR155) |
| | AGAACGGCCCUGUGCUUAAGACACUCCUGCUGCCCCGUCUGAGG | |
| | GUGGCAAUUAAAGUUGCUUCACAUCCUC | |
| 6 | AUGGCUAUCCUUUAAUGAUGCGUGUGGAAUGUGUGUGUGUGCU | HMGB2 |
| | CAGGCAAUUAUUUUGCUAAGAAUGUGAAUUCAAGUGCAGCUCAA | 3UTR047 |
| | UACUAGCUUCAGUAUAAAAACUGUACAGAUUUUUGUAUAGCUG | |
| | AUAAGAUUCUCUGUAGAGAAAAUACUUUUAAAAAAUGCAGGUU | |
| | GUAGCUUUUUGAUGGGCUACUCAUACAGUUAGAUUUUACAGCUU | |
| | CUGAUGUUGAAUGUUCCUAAAUAUUUAAUGGUUUUUUUAAUUU | |
| | CUUGUGUAUGGUAGCACAGCAAACUUGUAGGAAUUAGUAUCAA | |
| | UAGUAAAUUUUGGGUUUUUUAGGAUGUUGCAUUUCGUUUUUUU | |
| | AAAAAAAAUUUUGUAAUAAAAUUAUGUAUAUUAUUUCUAUUGU | |
| | CUUUGUCUUAAUAUGCUAAGUUAAUUUUCACUUUAAAAAAGCCA | |
| | UUUGAAGACCAGAGCUAUGUUGAUUUUUUUCGGUAUUUCUGCCU | |
| | AGUAGUUCUUAGACACAGUUGAC | |
| 7 | AUGGCUAUCCUUUAAUGAUGCGUGUGGAAUGUGUGUGUGUGCU | HMGB2 |
| | CAGGCAAUUAUUUUGCUAAGAAUGUGAAUUCAAGUGCAGCUCAA | 3UTR177 |
| | UACUAGCUUCAGUAUAAAAACUGUACAGAUUUUUGUAUAGCUG | |
| | AUAAGAUUCUCUGUAG | |
| 8 | AAGCCGGGUGGGGGUGAGGGUAGCCCUUGAGCCCUGUCCCUGCG | HMGB2 |
| | GCUGUGAGAGCAGCAGGACCCUGGGCCAGUUCCAGAGACCUGGG | UTR234 |
| | GGUGUGUCUGGGGGUGGGGUGUGAGUGCGUAUGAAAGUGUGUG | |
| | UCUGCUGGGGCAGCUGUGCCCCUGAAUCAUGGGCACGGAGGGCC | |
| | GCCCGCCACGCCCCGCGCUCAACUGCUCCCGUGGAAGAUUAAAG | |
| | GGCUGAAUCAUGGUGCUGA | |
| 9 | GGACACAUGGUUACCACCUGGCCCUGAGUGCAGUCAGACUGGGA | UTR320 |
| | CAAAGGAGCCGUGAAACACGCAAGGAGCUUCUGGCUUCUCAGCU | |
| | UUGAGACCUGGG | |
| 10 | GCUCUUUGGAGCACAGAGAACUUUGAUUUUUAAGCUUCUACAGA | UTR325 |
| | UAUGGUCCUAGAGACUGGGGUCCUCAGACACUCCCGCUGUAUGU | |
| | GGUGAGCUUGGCUGGAAACUUGCCAUAGCCAGGACUGAGGGGCU | |
| | GGCCCCAGGACGCUCUCAAGAAAUGGGGCUACUGUGGUUUAGAC | |
| | ACUCCUGCUGCUCACAUUGAUGGGUGGCUAUUAAAG | |
| 11 | GGGGUCCUCAGACACUCCCGCUGUAUGUGGUGAGCUUGGCUGGA | UTR326 |
| | AACUUGCCAUAGCCAGGACUGAGGGGCUGGCCCCAGGACGCUCU | |
| | CAAGAAAUGGGGCUACUGUGGUUUAGACACUCCUGCUGCUCACA | |
| | UUGAUGGGUGGCUAUUAAAG | |
| 12 | GCUCUUUGGAGCACAGAGAACUUUGAUUUUUAAGCUUCUACAGA | UTR341 |
| | UAUGGUCCUAGAGACUG | |
| 13 | GCUCUUUGGAGCACAGAGAACUUUGAUUUUUAAGCUUCUACAGA | UTR342 |
| | UAUGGUCCUAGAGACUGGGGUCCUCAGACACUCCCGCUGUAUGU | |
| | GGUGAGCUUGGCUGGAAACUUGC | |
| 14 | GCUCUUUGGAGCACAGAGAACUUUGAUUUUUAAGCUUCUACAGA | UTR343 |
| | UAUGGUCCUAGAGACUGGGGUCCUCAGACACUCCCGCUGUAUGU | |
| | GGUGAGCUUGGCUGGAAACUUGCCAUAGCCAGGACUGAGGGGCU | |
| | GGCCCCAGGACGCUCUCAAGAAAUGGGGCUACUGUGGUUUAGAC | |
| | ACUCC | |
| 15 | GCUCUUUGGAGCACAGAGAACUUUGAUUUUUAAGCUUCUACAGA | UTR344 |
| | UAUGGUCCUAGAGACUGGGGUCCUCAGACACUCCCGCUGUAUGU | |
| | GGUGAGCUUGGCUGGAAACUUGCCAUAGCCAGGACUGAGGGGCU | |
| | GGCCCCAGGACGCUCUCAAGAAAUGGGGCUACUGUGGUUUAGAC | |
| | ACUCCUGCUGCUCACAUUGAUGGGU | |
| 16 | CUUUGAUUUUUAAGCUUCUACAGAUAUGGUCCUAGAGACUGGGG | UTR371 |
| | UCCUCAGACACUCCCGCUGUAUGUGGUGAGCUUGGCUGGAAACU | |
| | UGCCAUAGCCAGGACUGAGGGGCUGGCCCCAGGACGCUCUCAAG | |
| | AAAUGGGGC | |
| 17 | CUUUGAUUUUUAAGCUUCUACAGAUAUGGUCCUAGAGACUGGGG | UTR372 |
| | UCCUCAGACACUCCCGCUGUAUGUGGUGAGCUUGGCUGGAAACU | |
| | UGCCAUAGCCAGGACUGAGGGGCUGGCCCCAGGACGCUCUCAAG | |
| | AAAUGGGGCUACUGUGGUU | |
| 18 | CUUUGAUUUUUAAGCUUCUACAGAUAUGGUCCUAGAGACUGGGG | UTR373 |
| | UCCUCAGACACUCCCGCUGUAUGUGGUGAGCUUGGCUGGAAACU | |
| | UGCCAUAGCCAGGACUGAGGGGCUGGCCCCAGGACGCUCUCAAG | |
| | AAAUGGGGCUACUGUGGUUUAGACACUCCUGCUGCUCAC | |
| 19 | UCUACAGAUAUGGUCCUAGAGACUGGGGUCCUCAGACACUCCCG | UTR374 |
| | CUGUAUGUGGUGAGCUUGGCUGGAAACUUGCCAUAGCCAGGACU | |
| | GAGGGGCUGGCCCCAGGACGCUCUCAAGAAAUGGGGC | |
| 20 | UCUACAGAUAUGGUCCUAGAGACUGGGGUCCUCAGACACUCCCG | UTR375 |
| | CUGUAUGUGGUGAGCUUGGCUGGAAACUUGCCAUAGCCAGGACU | |
| | GAGGGGCUGGCCCCAGGACGCUCUCAAGAAAUGGGGCUACUGUG | |
| | GUU | |
| 21 | UGGUCCUAGAGACUGGGGUCCUCAGACACUCCCGCUGUAUGUGG | UTR377 |
| | UGAGCUUGGCUGGAAACUUGCCAUAGCCAGGACUGAGGGGCUGG | |
| | CCCCAGGACGCUCUCAAGAAAUGGGGC | |
| 22 | UGGUCCUAGAGACUGGGGUCCUCAGACACUCCCGCUGUAUGUGG | UTR378 |
| | UGAGCUUGGCUGGAAACUUGCCAUAGCCAGGACUGAGGGGCUGG | |
| | CCCCAGGACGCUCUCAAGAAAUGGGGCUACUGUGGUU | |
| 23 | GGGGUCCUCAGACACUCCCGCUGUAUGUGGUGAGCUUGGCUGGA | UTR380 |
| | AACUUGCCAUAGCCAGGACUGAGGGGCUGGCCCCAGGACGCUCU | |
| | CAAGAAAUGGGGC | |
| 24 | GGGGUCCUCAGACACUCCCGCUGUAUGUGGUGAGCUUGGCUGGA | UTR381 |
| | AACUUGCCAUAGCCAGGACUGAGGGGCUGGCCCCAGGACGCUCU | |
| | CAAGAAAUGGGGCUACUGUGGUU | |
| 25 | ACAAGUAAGGGCUUGAAAAUGAUACUGGCAAGAUACGAUUGGC | 3UTR306 |
| | UCUAGAUCUACAUUCUUCAAAAAAAAAAAUUGGCUUAACUGUUU | |
| | CAUCUUUAAGUAGCAUUUUGCUGCCAUUUGUAUU | |
| 26 | GGGACAGGGUCAGGGCAGCCCAGGGCUCCUGGCUUCAGCAGGAA | 3UTR248 |
| | GUGAACAGGCUCAGGGAACUGGAGGAAGCGAAGCAUCAAGGCCA | |
| | GAGGAGGCCACAUGCUGACCAGCCUGAUGAGGCAAGAGCCUGCC | |
| | CCUGCCACCGCCCCGACCCCUCUCCUCUCUGCAAGAGCCUGCCUC | |
| | UGCCACCGCCCCGACCCCCUCUCCUCUCAGCAAGGGAUGGGCCUC | |
| | UCUGCCUCGCCCACCCCUCAGCCCUCCUCCCAGCCAUCUCCUCUU | |
| | CCCUAAGGCCUCUGUCUCCAUAGCUCUGGUUUCCCUGGGCCUCA | |
| | GUCCUCCCCACCCUCCUUCCUCUGUCUCCCUGUCACUAAUGUGAG | |
| | GUUUCUUUGUGCACAUUAAAGUCUUCUUUCAGCAUCA | |
| 27 | AGGGUUUCGUCUUUAGAUCUUCUGGUCCCCACAGACUCAGAGAG | 5UTR025 |
| | AACCCGCCACC | |
| 28 | GCGCAAAGGGGCUGAAAGAACAUGGACUUGUAUAUUUGUACAA | RNA397- |
| | AAAAAAAGUUUUAUUUUUCUAAAAAAAGAAAAAAGAAGAAAAA | 3UTR055 |
| | AUUUAAAGGGUGUACUUAUAUCCACACUGCACACUGCCUGGCCC | |
| | AAAACGUCUUAUUGUGGUAGGAUCAGCCCUCAUUUUGUUGCUUU | |
| | UGUGAACUUUUUGUAGGGGACGAGAAAGAUCAUUGAAAUUCUG | |
| | AGAAAACUUCUUUUAAACCUCACCUUUGUGGGGUUUUUGGAGAA | |
| | GGUUAUCAAAAAUUUCAUGGAAGGACCACAUUUUAUAUUUAUU | |
| | GUGCUUCGAGUGACUGACCCCAGUGGUAUCCUGUGACAUGUAAC | |
| | AGCCAGGAGUGUUAAGCGUUCAGUGAUGUGGGGUGAAAAGUUA | |
| | CUACCUGUCAAGGUUUGUGUUACCCUCCUGUAAAUGGUGUACAU | |
| | AAUGUAUUGUUGGUAAUUAUUUUGGUACUUUUAUGAUGUAUAU | |
| | UUAUUAAACAGAUUUUUACAAA | |
| 29 | AAUUGAUCAGGGACCAUGAAAAGAAACUUGUGCUUCACCGAAGA | RNA399- |
| | AAAAUAUCUAAACAUCGAAAAACUUAAAUAUUAUGGAAAAAAA | 3UTR054 |
| | ACAUUGCAAAAUAUAAAAUAAAUAAAAAAAGGAAAGGAAACUU | |
| | UGAACCUUAUGUACCGAGCAAAUGCCAGGUCUAGCAAACAUAAU | |
| | GCUAGUCCUAGAUUACUUAUUGAUUUAAAAACAAAAAAACACAA | |
| | AAAAAUAGUAAAAUAUAAAAACAAAUUAAUGUUUUAUAGACCC | |
| | UGGGAAAAAGAAUUUUCAGCAAAGUACAAAAAUUUAAAGCAUU | |
| | CCUUUCUUUAAUUUUGUAAUUCUUUACUGUGGAAUAGCUCAGAA | |
| | UGUCAGUUCUGUUUUAAGUAACAGAAUUGAUAACUGAGCAAGG | |
| | AAACGUAAUUUGGAUUAUAAAAUUCUUGCUUUAAUAAAAAUUC | |
| | CUUAAACAGUG | |
| 30 | UCCCCAGACCUCUGUCCCUGUUCCCCUCCACUCCUCCCCUCACUC | RNA401- |
| | CCCUGCUCCCCCGACCACCUCCUCCUCUGCCUCAAAGACUCUUGU | 3UTR052 |
| | CCUCUUGUCCCUCCUGAGAAAAAAGAAAACGAAAAGUGGGGUUU | |
| | UUUUCUGUUUUCUUUUUUUCCCCUUUCCCCCUGCCCCCACCCACG | |
| | GGGCCUUUUUUUGGAGGUGGGGGCUGGGGAAUGAGGGGCUGAG | |
| | GUCCCGGAAGGGAUUUUAUUUUUUUGAAUUUUAAUUGUAACAU | |
| | UUUUAGAAAAAGAACAAAAAAAGAAAAAAAAAAGAAAGAAACA | |
| | CAGCAACUGUAGAUGCUCCUGUUCCUGGUUCCCGCUUUCCACUU | |
| | CCAAAUCCCUCCCCUCACCUUCCCCCACUGCCCCCCAAGUUCCAG | |
| | GCUCAGUCUUCCAGCCGCCUGGGGAGUCUCUACCUGGGCCCAAG | |
| | CAGGUGUGGGGCCUCCUUCUGGGCUUUUCUUCUGAAUUUAGAGG | |
| | AUUUCUAGAACGUGG | |
| 31 | GCGCAAAGGGGCUGAAAGAACAUGGACUUGUAUAUUUGUACAA | RNA403- |
| | AAAAAAAGUUUUAUUUUUCUAAAAAAAGAAAAAAGAAGAAAAA | 3UTR050 |
| | AUUUAAAGGGUGUACUUAUAUCCACACUGCACACUGCCUGGCCC | |
| | AAAACGUCUUAUUGUGGUAGGAUCAGCCCUCAUUUUGUUGCUUU | |
| | UGUGAACUUUUUGUAGGGGACGAGAAAUGAGGGGCUGAGGUCC | |
| | CGGAAGGGAUUUUAUUUUUUUGAAUUUUAAUUGUAACAUUUUU | |
| | AGAAAAAGAACAAAAAAAGAAAAAAAAAAGAAAGAAACACAGC | |
| | AACUGUAGAUGCUCCUGUUCCUGGUUCCCGCUUUCCACUUCCAA | |
| | AUCC | |
| 32 | UGGAUGUACAUCAUUCCCGUCGUCCUGUUCCUCAUGAUGUCAGG | RNA373- |
| | AGCGCCAGACACCGGGGGCCAGGGUGGGGGUGGGGGUGGGGGUG | 3UTR068 |
| | GUGGUGGGGGUAGUGGCCGGUGAGGGCCCAGGCUGGUCAGCGUC | |
| | CCGUCUUGCACACCCAGGGGCCUCCCUUUCUGCUGGAGUCCCCU | |
| | GUGUCCUCAGCCAUCCCAAGAAGG | |
| 33 | GCAAAACUGCCGCAAGUCUGCAGCCCGGCGCCACCAUCCUGCAG | RNA375- |
| | CCUCCUCCUGACCACGGACGUUUCCAUCAGGUUCCAUCCCGAAA | 3UTR063 |
| | AUCUCUCGGUUCCACGUCCCCCUGGGGCUUCUCCUGACCCAGUCC | |
| | CCGUGCCCCGCCUCCCCGAAACAGGCUACUCUCCUCGGCCCCCUC | |
| | CAUCGGGCUGAGGAAGCACAGCAGCAUCUUCAAACAUGUACAAA | |
| | AUCGAUUGGCUUUAAACACCCUUCACAU | |
| 34 | UGAAAACCAAAAUCCAUGCAAAUGAAAUGUAAUUGGCACGACCC | RNA377- |
| | UCACCCCCAAAUCUUACAUCUCAAUUCCCAUCCUAAAAAGCACU | 3UTR065 |
| | CAUACUUUAUGCAUCCCCGCAGCUACACACACACAACACACAGC | |
| | ACACGCAUGAACACAGCACACACACGAGCACAGCACACACACAA | |
| | ACGCACAGCACACACAGCACACAGAUGAGCACACAGCACACACA | |
| | CAAACGCACAGCACACACACGCACACACAU | |
| 35 | CACACACACGCACACACAUGCACACACAGCACACAAACGCACGGC | RNA379- |
| | ACACACACGCACACACAUGCACACACAGCACACACACAAACGCAC | 3UTR064 |
| | AGCACACACAAACGCACAGCACACACGCACACACAGCACACACAC | |
| | GAGCACACAGCACACAAACGCACAGCACACGCACACACAUGCAC | |
| | ACACAGCACACACACUAGCACACAGCACACACACAAAGACACAG | |
| | CACACACAUGCACACACAGCACACACACGCGAACACAGCACACAC | |
| | GAACACAGCACACACAGCACACACACAAACACAGCACACACAUG | |
| | CACACAGCACACGCACACACAGCACACACAUGAACACAGCACAC | |
| | AGCACACACAUGCACACACAGCACACACGCAUGCACAGCACACA | |
| | UGAACACAGCACACACACAAACACACAGCACACACAUGCACACA | |
| | CAGCACACACACUCAUGCGCAGCACAUACAUGAACACAGCUCAC | |
| | AGCACACAAACACGCAGCACACACGUUGCACACGCAAGCACCCA | |
| | CCUGCACACACACAUGCGCACACACACGCACACCCCCACAAAAUU | |
| | GGAUGAAAACAAUAAGCAUAUCUAAGCAACUACGAUAUCUGUAU | |
| | GGAUCAGGCCAAAGUCCCGCUAAGAUUCUCCAAUGUUUUCAUGG | |
| | UCUGAGCCCCGCUCCUGUUCCCAUCUCCACUGCCCCUCGGCCCUG | |
| | UCUGUGCCCUGCCUCUCAGAGGAGGGGGCUCAGAUGGUGCGGCC | |
| | UGAGUGUGCGGCCGGCGGCAUUUGGGAUACACCCGUAGGGUGGG | |
| | CGGGGUGUGUCCCAGGCCUAAUUCCAUCUUUCCACCAUGACAGA | |
| | GAUGCCCUUGUGAGGCUGGCCUCCUUGGCGCCUGUCCCCACGGC | |
| | CCCCGCAGCGUGAGCCACGAUGCUCCCCAUACCCCACCCAUUCCC | |
| | GAUACACCUUACUUACUGUGUGUUGGCCCAGCCAGAGUGAGGAA | |
| | GGAGUUUGGCCACAUUGGAGAUGGC | |
| 36 | GUCGCAUGAAUGCCAUCUCUGGCUGGCAGGCCUUCUUUCCAGUC | RNA371- |
| | CAUUUCCAGGAGUUCAAUCCUGCCCUGUCACCACAGAGAUCACC | 3UTR067 |
| | CCCAGGGCCCCCGGGGGCUGGCCCUGACCCCCCCUCCCCUCCUGG | |
| | UGCUGACCCCUCCCGGGGGGCUCCUAUAGGGGGGAGAUUUGACC | |
| | GGCAGGCUUCUGCGGAGGGCUGCUUCUACAACGCUGACUACCUG | |
| | GCGGCCCGAGCCCGGCUGGCAGGUGAACUGGCAGGCCAGGAAGA | |
| | GGAGGAAGCCCUGGAGGGGCUGGAGGUGAUGGAUGUUUUCCUCC | |
| | GGUUCUCAGGGCUCCACCUCUUUCGGGCCGUAGAGCCAGGGCUG | |
| | GUGCAGAAGUUCUCCCUGCGAGACUGCAGCCCACGGCUCAGUGA | |
| | AGAACUCUACCACCGCUGCCGCCUCAGCAACCUGGAGGGGCUAG | |
| | GGGGCCGUGCCCAGCUGGCUAUGGCUCUCUUUGAGCAGGAGCAG | |
| | GCCAAUAGCACUUAGCCCGCCUGGGGGCCCUAACCUCAUUACCU | |
| | UUCCUUUGUCUGCCUCAGCCCCAGGAAGGGCAAGGCAAGAUGGU | |
| | GGACAGAUAGAGAAUUGUUGCUGUAUUUUUUAAAUAUGAAAAU | |
| | GUUAUUAAACAUGUCUUCUGCCAAA | |
| 37 | UUCUGGAAUCUGUGCUCUGGGGGCUGUGCCGGGUAGAGAGGGCA | RNA369- |
| | GUGGGAGGUAAGAGCUCUUCACCCUUCACCACCUUCUCCACCCA | 3UTR069 |
| | GCAUGGCCGGCA | |
| 38 | GUUUAGCUUUGUGUUAGCUUAUACAUACUAAAACCUUUAAAAA | RNA365- |
| | GCUUUUCUUCUCAAUUGAUUUUUUUCUUUUAGAAGCCAUGGUGU | 3UTR070 |
| | CUCAACCUUUUGGGGACCUAACUUCUAAACAUUCUAAUAGUUUG | |
| | CCUUAAUUUUUCUUCUGCUUUCUUACUAAAAAUGAAGACAUUCA | |
| | AUACUAAUCUUGCUGGAAGAAGCCUUAACCAAGCAAACUUCUCA | |
| | UUUCUCUGGUGAAAACUGCUGCCAAAACCACUUGUUAAAAAUUG | |
| | UACAGAGCCUGUAGAAAAUAUAGAAGAUUCAUUGGAUGUUGGC | |
| | CUAGUUCUGUGUGGAAGACUAGUGAUUUUGUUGUUUUUAGAUA | |
| | ACUAAAUCGACAACAAAUCACAGUCUGCCAUAUGGCACAGGCCA | |
| | UGCCUCUACAGGACAAAUGAUUGGUGCUGUAAAAUGCAGCAUUU | |
| | CACACCUUACUAGCAUUCUUUGUCUUUUCUACCAAAUAUUAACA | |
| | ACUUUCAAUUCCGUUUUCUUAAUUCUGUUCUACUAAUGUCCGAU | |
| | UUACUACUCAUCAUUUUUCUUGACACUUAACAUUGCUUUAAUUU | |
| | GUAAUUGCUAAUGGUUUUUGAACUCUUCUAAUUGUAAUGGACG | |
| | UGUUUAUCAUUUUAAUUUAGCAUUGAAAUUGUCUUGAUGUUGA | |
| | U | |
| 39 | CCUGAGGCUGGGUUGGAGCAGCCCUCCUGUGCCUGAGGCCAGCU | RNA367- |
| | CCCAGGCCCUUGGAUCACCGCGGGAGGAACCCUCAGGAUGGGUG | 3UTR066 |
| | GAGCCUCCAGGCUAUGGGCAUUGCCUGCCUGAUGCCAGCACCAC | |
| | CUGGGCUGGGCCCUGGGCUUGGCUCGAGUUCUCCUGCUGGUGAG | |
| | GCUCCGGAUCUCAGGAGCAGCCCUGAGUCUGCUUCCCAGGCUGC | |
| | CCCUGCCAGGCCUGCAGCCUCCCCAGCCAGGGCUGCUCUCUGCUG | |
| | UCCCCAUUCAGUGCCCUGGCCCCUGCAUUCAUGCCCCCCACACCC | |
| | CCUCAGGCCCUGUGCCUGGACUUUGGGGCUGGCAGCUGAAGCCU | |
| | UGAGAUCCUGGGCCAGCUGCCGGCACACAGCUAGGCAGACUCUC | |
| | CCACCAGGUGCCCCUGCCCAGGCCUCCUAAUCGGGGGCAGACAG | |
| | GCAGGGAGGGUGUGGCUGGGCUGGGCUGGGCGGGGCGGCCUGGG | |
| | GCAGGGGUGUGGCCCCUAAAUGUCCCCAACCUCAGAGGGACCUA | |
| | GAGUCCUGAGCCUCCAGUAGCUUCUCUGGGCCUGGCAGAGGUAA | |
| | GGGGGAGGCAACCCUGGAGUGUCUGGAGGCCCAUGGCUGGCUGA | |
| | ACCCUGGAUGCCUUUUCUUCCGCGUCCCCAUGAAUGAAAGCUGU | |
| | CUGGGCCUUCAUUCUGCAGACAGGGACAAACAGCUCCAUGCUGU | |
| | UUGUCCUCCCAGUGCAGCCGUGCUGGGAGGGUCUGGGGGAGCUU | |
| | CCUACAAGGAGAGACUCCUGCUGCUUUGGAAAACUGAGAAAAAA | |
| | UAGGGGUCUAACCCUCUCCUCCCAUUUUACAAGUGGGGAAAUGA | |
| | GGCGUGAAAGGAGAGGCGUCUGGGUUACUCCGUGGGUCUGGGGU | |
| | CCAGGGAAGGGCCUGUAUGGGGGAGGGAGCUGGGAGGGGACGG | |
| | UGUCUGGCUCUACCCCUGUGGGGUGGGGAGGUGGGGCUCCCCUG | |
| | UAUCACAGGACAUCCCCCCUGAGAGGUCCCUCAUAUGUCUGGGU | |
| | CCUGUGGGUGGGGGACUAACUGCGCAAUGUAGUUAGGUGCUCAA | |
| | UAAACGGAGUUGCCGCUGA | |
| 40 | UGUUCAGAGGUCCCUGUCUUCUGUCCCCAUCUUCCUGCCGAUAG | RNA385- |
| | CUAUCCCCUGUAUGAUGUUGGAUGCUCCUCACAUGCUGAGUUUC | 3UTR059 |
| | CAGCCUUUUCUGAAACUCAGUAGCUGGGGAGAGGGCAGGGAGGC | |
| | UUCCUGGGCCUUCCAGCCUCCUUCCCCACCUCCUUCCCAAACCCU | |
| | CUUGGGAACUCCUCAGGGACAACUACUGCUGAGUUUGGGUGCAC | |
| | CCUAAGAUGGAGGCCAGGUAGCAAUGGGGCCGGCCUCAGAGAGA | |
| | GCGCUGUGUGUGUGUGUGUGUGUGUGUGUGUGUGUGUGUGUCG | |
| | GCCUCAGAGCGCGCUGUGUGUGUGUGUGUGUGUGUCAGCCUCAG | |
| | AGCGCGCGCUGUGUGUGUGUGUGUGUGUGCGUGCUUGUGACCCU | |
| | GUAUUGUUUGAUAGGAUCCAUUCAGUUUCCCCAAGUACCUGUUU | |
| | UCAUUCCCAUUUUUCCCAUUGUUUAAAACCAUCACUUUUUUGUC | |
| | UUUGGGAAACCACAGG | |
| 41 | CUUUUCACAAGAUGGACCCUUCAUUUCAAGCUUAGGCUGGCGUU | RNA381- |
| | ACUUUUGCUGUCUAGUCAGGACUAAUCACGGUGUUUCAGUGCGG | 3UTR061 |
| | AGUGCCAAGAGUCCUAUCCUGACGUCAGGCUCUGGGUGUCAACC | |
| | UCUGACUUAUUCUGCAGAUGCUCUGUGUGUGUGUGUGUGUGUG | |
| | UGUGUGUGUGUGUGUGUGUGUGUGUGUUCGGGGAGAGGGUGGU | |
| | AGCACAGGGCUUGGGAUAUCGGCAGUGUGGGAAAUGCGAAGCAU | |
| | UUCUCAUCAUCAUCAUCUCUGCUACAGUCAUGUUUCUGCAUGUC | |
| | AGCGAGCGACACUGUCCCUGCCUCAGGUUGGAGGUUUUAUCAGC | |
| | CAAAGUGUUUUUUUCAUGUAUCGUUCGUUCCAUUCAUCCACUCU | |
| | GUGCCUUGUCAGCCUUUGAAAGGCUUGGUUGCUCCCAGGCUGCU | |
| | GUUCUCAGGGACCUUAAAAGGGACCUGGUUAGUCUUGGGGCAGA | |
| | GAGUAUCUACUUGGGCACCUUUUCACAAGAUGGACCCUUCAUUU | |
| | CAAGCUUAGGCUGGCGUUACUUUUGCUGUCUAGUCAGGACUAAU | |
| | CACGGUGUUUCAGUGCGGAGUGCCAAGAGUCCUAUCCUGACGUC | |
| | AGGCUCUGGGUGUCAACCUCUGACUUAUUCUGCAGAUGCUCUGU | |
| | GUGUGUGUGUGUGUGUGUGUGUGUGUGUGUGUGUGUGUGUGUG | |
| | UUCGGGGAGAGGGUGGUAGCACAGGGCUUGGGAUAUCGGCAGU | |
| | GUGGGAAAUGCGAAGCAUUUCUCAUCAUCAUCAUCUCUGCUACA | |
| | GUCAUGUUUCUGCAUGUCAGCGAGCGACACUGUCCCUGCCUCAG | |
| | GUUGGAGGUUUUAUCAGCCAAAGUGUUUUUUUCAUGUAUCGUU | |
| | CGUUCCAUUCAUCCACUCUGUGCCUUGUCAGCCUUUGAAAGGCU | |
| | UGGUUGCUCCCAGGCUGCUGUUCUCAGGGACCUUAAAAGGGACC | |
| | UGGUUAGUCUUGGGGCAGAGAGUAUCUACUUGGGCAC | |
| 42 | GUGUGUGUACAUCUGGGUGUGUGUGAGCAAGUGUGCAGAGGGU | RNA383- |
| | GCGUGUGCACAGGUACGGGCAGGUGCAUGUGUGCGAGUGGCUGU | 3UTR060 |
| | GUGAGUGGGUGUGUGAGAAGGUGAAUGUGAGAGCUGGUGUGGC | |
| | UGAGAGUGGGUGUGGCCAUGUUUGUGUGUCUGUCAACACAUGA | |
| | AUGUAUUUCAGUGCGUGUGAGCAGCUGUGCACGUAUGAUGUCAG | |
| | UGGGUGUCAGCAGUGUGUGGGUGUGCCUGUGUGAGCAGGGAUG | |
| | UGAGCAUGGUAUAUGUGAGUGGCUGUAAGCAGGCAUGUGUGAG | |
| | UGAUAUGUGUGAGUGGGGGUGUGACAGCAGGUGUGUGAGCAAG | |
| | UGUAUAUGUGUGAAUGUGUAUGUAAGCAGGGAUGUGUGUGAUA | |
| | UAAGUGUGUGUGAAUGGGCAUAGGUGUGU | |
| 43 | CUUUUCACAAGAUGGACCCUUCAUUUCAAGCUUAGGCUGGCGUU | RNA387- |
| | ACUUUUGCUGUCUAGUCAGGACUAAUCACGGUGUUUCAGUGCGG | 3UTR062 |
| | AGUGCCAAGAGUCCUAUCCUGACGUCAGGCUCUGGGUGUCAACC | |
| | UCUGACUUAUUCUGCAGAUGCUCUGUGUGUGUGUGUGUGUGUG | |
| | UGUGUGUGUGUGUGUGUGUGUGUGUGUUCGGGGAGAGGGUGGU | |
| | AGCACAGGGCUUGGGAUAUCGGCAGUGUGGGAAAUGCGAAGCAU | |
| | UUCUCAUCAUCAUCAUCUCUGCUACAGUCAUGUUUCUGCAUGUC | |
| | AGCGAGCGACACUGUCCCUGCCUCAGGUUGGAGGUUUUAUCAGC | |
| | CAAAGUGUUUUUUUCAUGUAUCGUUCGUUCCAUUCAUCCACUCU | |
| | GUGCCUUGUCAGCCUUUGAAAGGCUUGGUUGCUCCCAGGCUGCU | |
| | GUUCUCAGGGACCUUAAAAGGGACCUGGUUAGUCUUGGGGCAGA | |
| | GAGUAUCUACUUGGGCAC | |
| 44 | CUAGGUGCAGAGAGCCCAGGCCUUAUGUUAAAAUCAUGCACUUG | RNA391- |
| | AAAAGCAAACCUUAAUCUGCAAAGACAGCAGCAAGCAUUAUACG | 3UTR058 |
| | GUCAUCUUGAAUGAUCCCUUUGAAAUUUUUUUUUUGUUUGUUU | |
| | GUUUAAAUCAAGCCUGAGGCUGGUGAACAGUAGCUACACACCCA | |
| | UAUUGUGUGUUCUGUGAAUGCUAGCUCUCUUGAAUUUGGAUAU | |
| | UGGUUAUUUUUUAUAGAGUGUAAACCAAGUUUUAUAUUCUGCA | |
| | AUGCGAACAGGUACCUAUCUGUUUCUAAAUAAAACUGUUUACAU | |
| | UCAUUAUGGGGUAUGUAUGACCUUCAUUUUCCAAGAAAUAGAAC | |
| | UCUAGCUUAGAAUUAUGGAUGCUCUAAAAUGUCAGAAUGGGAA | |
| | CUCUCCUCGAAGUUCUCCCAAACUCAGAGACAGCACUGCCUUCU | |
| | CCUAAAUGAUUAUUCUUUUCUCCCUGUUUUCUGGUAUUUUCUAG | |
| | CAUCCUUCUCACCACAGCCA | |
| 45 | AGGGAGCCAUGAGGGUCUGGGCUUCAGAGCUAGGUCUUUGGGGA | RNA393- |
| | AGUCCUGGCUGACUGCCUUAGCAGUGGGGGUGGGGGUGGGGGCA | 3UTR056 |
| | GGGGCAGGGGCUUUAUGUGUUUUUGCUUGGGGGGCGCUGGGCCU | |
| | AGCCCAGAGUAGUGCUUGCUCCCCCUGCCUUGUCCCACCAGGGA | |
| | GGCAGCAGACUCAGGCCCUCCAUGGUCCUCUUUGUCAUUUUGUU | |
| | GACAUGCAUUCCUCCUUUUGUCAUCUUGUUGGGGGGAGGGGAUU | |
| | AACCAAAGGCCACCCUGACUUUGUUUUUGUGGACACACAAUAAA | |
| | AGCCCCGUUUAUUUGUAA | |
| 46 | ACACACACACAACACACAGCACACGCAUGAACACAGCACACACA | RNA395- |
| | CGAGCACAGCACACACACAAACGCACAGCACACACAGCACACAG | 3UTR053 |
| | AUGAGCACACAGCACACACACAAACGCACAGCACACACACGCAC | |
| | ACACAUGCACACACAGCACACAAACGCACGGCACACACACGCAC | |
| | ACACAUGCACACACAGCACACACACAAACGCACAGCACACACAA | |
| | ACGCACAGCACACACGCACACACAGCACACACACGAGCACACAGC | |
| | ACACAAACGCACAGCACACGCACACACAUGCACACACAGCACAC | |
| | ACACUAGCACACAGCACACACACAAAGACACAGCACACACAUGC | |
| | ACACACAGCACACACACGCGAACACAGCACACACGAACACAGCA | |
| | CACACAGCACACACACAAACACAGCACACACAUGCACACAGCAC | |
| | ACGCACACACAGCACACACAUGAACACAGCACACAGCACACACA | |
| | UGCACACACAGCACACACGCAUGCACAGCACACAUGAACACAGC | |
| | ACACACACAAACACACAGCACACACAUGCACACACAGCACACAC | |
| | ACUCAUGCGCAGCACAUACAUGAACACAGCUCACAGCACACAAA | |
| | CACGCAGCACACACGUUGCACACGCAAGCACCCACCUGCACACAC | |
| | ACAUGCGCACACACACGCACACCCCCACAAAAUUGGAUGAAAAC | |
| | AAUAAGCAUAUCUAAGCAACUACGAUAUCUGUAUGGAUCAGGCC | |
| | AAAGUCCCGCUAAGAUUCUCCAAUGUUUUCAUGGUCUGAGCCCC | |
| | GCUCCUGUUCCCAUCUCCACUGCCCCUCGGCCCUGUCUGUGCCCU | |
| | GCCUCUCAGAGGAGGGGGCUCAGAUGGUGCGGCCUGAGUGUGCG | |
| | GCCGGCGGCAUUUGGGAUACACCCGUAGGGUGGGGGGGGUGUGU | |
| | CCCAGGCCUAAUUCCAUCUUUCCACCAUGACAGAGAUGCCCUUG | |
| | UGAGGCUGGCCUCCUUGGCGCCUGUCCCC | |
| 47 | GGGUUGCAGACACAUAUAUUUUUGAGGCUGGGUGACGAGAAAA | RNA389- |
| | UCUAGAGACAUGAGGGACAUAAAUGGGCCUGGCAGCCUCGGCUC | 3UTR057 |
| | UUUGCGGCUGCUGGCAGGACUGAGCUGUCCGGGUUCUCCCCACA | |
| | CUUCCAGCACAGCUGUGCUCUGUGUCCUGCCUCGGCGCUCUCGC | |
| | AAAUGAAGCUGCAGGCCAAGAA | |
| 48 | GGGUGUGCCUCCACAAGUGUGCAAGGGUAUAUGUAUAUAUUUGC | RNA407- |
| | AGGCAUGUGUGAGGCGCACUAGUAAGUACAUAAUUUGCUGGCAU | 3UTR051 |
| | GAUUGCAGGUAGGCAUGAGUGUGUAUGUGCUAACAUGUAGGCU | |
| | GGUGUGUGUAGGCAUGUGUGAAUAAGCAUGUAUAAAUGAGCUU | |
| | AAGUGUGCUGUGUGCACAUGUGUACACACAGCUAAUUUAUCUGC | |
| | AGACCUAUAUGUGAGCAUGUAAGAGUGAACAUAUGUGUGUGUG | |
| | UGUAGUAUGUAAGAAUGAGGAACUCGGUAUGUGCAUGUGUAGA | |
| | CAGGUACCCAGCAGUGUGUGUAUAUGCGCAUGAGUGAGGAUGUA | |
| | UAGGCAGAAGAUGUGUGUGUCUAUGAGUGAGUUUGAGUGUGCA | |
| | GGCUUGUAUUAGGUGUAUGUGAGGAAGCCCUUGUGUGUGCAGG | |
| | GGUGCACAUGUUUGGCCACAGGCAUGGGAGGUGUAUGUUAGGA | |
| | GCAUGUGUGUUUGUAGGCAGACUCAUGAGCAGGUGUGUGCAAA | |
| | UACAUAUCCAGCUGCACUGUGGGUGUCCACCCACACCUUGUGUU | |
| | CCUCAUGGCCUACCCCAGCCUUUCUUCUCCACUGGGUCCCACUGU | |
| | UCCCUGGAGACAGAGGGCUAGCAUGCUGUCAUUUAUCUGAA | |
| 49 | AUGGCUAUCCUUUAAUGAUGCGUGUGGAAUGUGUGUGUGUGCU | RNA409- |
| | CAGGCAAUUAUUUUGCUAAGAAUGUGAAUUCAAGUGCAGCUCAA | 3UTR047 |
| | UACUAGCUUCAGUAUAAAAACUGUACAGAUUUUUGUAUAGCUG | |
| | AUAAGAUUCUCUGUAGAGAAAAUACUUUUAAAAAAUGCAGGUU | |
| | GUAGCUUUUUGAUGGGCUACUCAUACAGUUAGAUUUUACAGCUU | |
| | CUGAUGUUGAAUGUUCCUAAAUAUUUAAUGGUUUUUUUAAUUU | |
| | CUUGUGUAUGGUAGCACAGCAAACUUGUAGGAAUUAGUAUCAA | |
| | UAGUAAAUUUUGGGUUUUUUAGGAUGUUGCAUUUCGUUUUUUU | |
| | AAAAAAAAUUUUGUAAUAAAAUUAUGUAUAUUAUUUCUAUUGU | |
| | CUUUGUCUUAAUAUGCUAAGUUAAUUUUCACUUUAAAAAAGCCA | |
| | UUUGAAGACCAGAGCUAUGUUGAUUUUUUUCGGUAUUUCUGCCU | |
| | AGUAGUUCUUAGACACAGUUGAC | |
| 50 | CAACUCAGCUCACAUCACCAGCUCACCUCUGGUAGCCAUAGCAG | RNA405- |
| | CCCCUGCUUCAGCCCCACCGCACCCCUCCAGGGGGCCUGCCUUUC | 3UTR049 |
| | CCUGACACUUUUGGGGUCUGCCUGGGGGAGGAGGGGAGAAAGCA | |
| | CCAUGAGUGCUCACUAAAACAACUUUUUCCAUUUUUAAUAAAAC | |
| | GCCAAAAAUAUCACAACCCACCAAAAAUAGAUGCCUCUCCCCCU | |
| | CCAGCCCUAGCCGAGCUGGUCCUAGGCCCCGCCUAGUGCCCCACC | |
| | CCCACCCACAGUGCUGCACUCCUCCUGCCCCUGCCACGCCCACCC | |
| | CCUGCCCACCUCUCCAGGCUCUGCUCUGCAGCACACCCGUGGGUG | |
| | ACCCCUCACCCCAGAAGCAGCAGUGGCAGCUUGGGAAAUGUGAG | |
| | GAAGGGAAGGAGGGAGAGACGGGAGGGAGGAGAGAGAGGAGAA | |
| | GGGAGGCAGGGGAGGGGCAGCAGAACCAAGGCAAAUAUUUCAGC | |
| | UGGGCUAUACCCCUCUCCCCAUCCCUGUUAUAGAAGCUUAGAGA | |
| | GCCAGCCAGCAAUGGAACCUUCUGGUUCCUGCGCCAAUCGCCAC | |
| | CAGUAUCAAUUGUGUGAGCUUGGGUGCGAGUGCACGCGUGCGUG | |
| | AGUACGGAGAGUAUAUAUAGAUCUCUAUCUCUUAGCAAAGGUG | |
| | AAUGCCAGAUGUAAAUGGCGCCUCUGGGCAAAGGAGGCUUGUAU | |
| | UUUGCACAUUUUAUAAAAACUUGAGAGAAUGAGAUUUCUGCUU | |
| | GUAUAUUUCUAAAAAGAGGAAGGAGCCCAAACCAUCCUCUCCUU | |
| | ACCACUCCCAUCCCUGUGAGCCCUACCUUACCCCUCUGCCCCUAG | |
| | CCAAGGAGUGUGAAUUUAUAGAUCUAACUUUCAUAGGCAAAACA | |
| | AAAGCUUCGAGCUGUUGCGUGUGUGAGUCUGUUGUGUGGAUGU | |
| | GCGUGUGUGGUCCCCAGCCCCAGACUGGAUUGGAAAAGUGCAUG | |
| | GUGGGGGCCUCGGGGCUGUCCCCACGCUGUCCCUUUGCCACAAG | |
| | UCUGUGGGGCAAGAGGCUGCAAUAUUCCGUCCUGGGUGUCUGGG | |
| | CUGCUAACCUGGCCUGCUCAGGCUUCCCACCCUGUGCGGGGCAC | |
| | ACCCCCAGGAAGGGACCCUGGACACGGCUCCCACGUCCAGGCUU | |
| | AAGGUGGAUGCACUUCCCGCACCUCCAGUCUUCUGUGUAGCAGC | |
| | UUUAACCCACGUUUGUCUGUCACGUCCAGUCCCGAGACGGCUGA | |
| | GUGACCCCAAGAAAGGCUUCCCCGACACCCAGACAGAGGCUGCA | |
| | GGGCUGGGGCUGGGUGAGGGUGGCGGGCCUGCGGGGACAUUCUA | |
| | CUGUGCUAAAAAGCCACUGCAGACAUAGCAAUAAAAACAUGUCA | |
| | UUUUCCAAA | |
| 51 | GGGUGUGCCUCCACAAGUGUGCAAGGGUAUAUGUAUAUAUUUGC | RNA411- |
| | AGGCAUGUGUGAGGCGCACUAGUAAGUACAUAAUUUGCUGGCAU | 3UTR048 |
| | GAUUGCAGGUAGGCAUGAGUGUGUAUGUGCUAACAUGUAGGCU | |
| | GGUGUGUGUAGGCAUGUGUGAAUAAGCAUGUAUAAAUGAGCUU | |
| | AAGUGUGCUGUGUGCACAUGUGUACACACAGCUAAUUUAUCUGC | |
| | AGACCUAUAUGUGAGCAUGUAAGAGUGAACAUAUGUGUGUGUG | |
| | UGUAGUAUGUAAGAAUGAGGAACUCGGUAUGUGCAUGUGUAGA | |
| | CAGGUACCCAGCAGUGUGUGUAUAUGCGCAUGAGUGAGGAUGUA | |
| | UAGGCAGAAGAUGUGUGUGUCUAUGAGUGAGUUUGAGUGUGCA | |
| | GGCUUGUAUUAGGUGUAUGUGAGGAAGCCCUUGUGUGUGCAGG | |
| | GGUGCACAUGUUUGGCCACAGGCAUGGGAGGUGUAUGUUAGGA | |
| | GCAUGUGUGUUUGUAGGCAGACUCAUGAGCAGGUGUGUGCAAA | |
| | UACAUAUCCAGCUGCACUGUGGGUGUCCACCCACACCUUGUGUU | |
| | CCUCAUGGCCUACCCCAGCCUUUCUUCUCCACUGGGUCCCACUGU | |
| | UCCCUGGAGACAGAGGGCUAGCAUGCUGUCAUUUAUCUGAAUUC | |
| | UGGUUCCUGCGCCAAUCGCCACCAGUAUCAAUUGUGUGAGCUUG | |
| | GGUGCGAGUGCACGCGUGCGUGAGUACGGAGAGUAUAUAUAGA | |
| | UCUCUAUCUCUUAGCAAAGGUGAAUGCCAGAUGUAAAUGGCGCC | |
| | UCUGGGCAAAGGAGGCUUGUAUUUUGCACAUUUUAUAAAAACU | |
| | UGAGAGAAUGAGAUUUCUGCUUGUAUAUUUCUAAAAAGAGGAA | |
| | GGAGCCCAAACCAUCCUCUCCUUACCACUCCCAUCCCUGUGAGCC | |
| | CUACCUUACCCCUCUGCCCCUAGCCAAGGAGUGUGAAUUUAUAG | |
| | AUCUAACUUUCAUAGGCAAAACAAAAGCUUCGAGCUGUUGCGUG | |
| | UGUGAGUCUGUUGUGUGGAUGUGCGUGUGUGGUCCCCAGCCCCA | |
| | GACUGGAUUGGAAAAGUGCAUGGUGGGGGCCUCGGGGCUGUCCC | |
| | CACGCUGUCCCUUUGCCACAAGUCUGUGGGGCAAGAGGCUGCAA | |
| | UAUUCCGUCCUGGGU | |
| 52 | GCGCAAAGGGGCUGAAAGAACAUGGACUUGUAUAUUUGUACAA | RNA445- |
| | AAAAAAAGUUUUAUUUUUCUAAAAAAAGAAAAAAGAAGAAAAA | 3UTR055 |
| | AUUUAAAGGGUGUACUUAUAUCCACACUGCACACUGCCUGGCCC | |
| | AAAACGUCUUAUUGUGGUAGGAUCAGCCCUCAUUUUGUUGCUUU | |
| | UGUGAACUUUUUGUAGGGGACGAGAAAGAUCAUUGAAAUUCUG | |
| | AGAAAACUUCUUUUAAACCUCACCUUUGUGGGGUUUUUGGAGAA | |
| | GGUUAUCAAAAAUUUCAUGGAAGGACCACAUUUUAUAUUUAUU | |
| | GUGCUUCGAGUGACUGACCCCAGUGGUAUCCUGUGACAUGUAAC | |
| | AGCCAGGAGUGUUAAGCGUUCAGUGAUGUGGGGUGAAAAGUUA | |
| | CUACCUGUCAAGGUUUGUGUUACCCUCCUGUAAAUGGUGUACAU | |
| | AAUGUAUUGUUGGUAAUUAUUUUGGUACUUUUAUGAUGUAUAU | |
| | UUAUUAAACAGAUUUUUACAAA | |
| 53 | AAUUGAUCAGGGACCAUGAAAAGAAACUUGUGCUUCACCGAAGA | RNA447- |
| | AAAAUAUCUAAACAUCGAAAAACUUAAAUAUUAUGGAAAAAAA | 3UTR054 |
| | ACAUUGCAAAAUAUAAAAUAAAUAAAAAAAGGAAAGGAAACUU | |
| | UGAACCUUAUGUACCGAGCAAAUGCCAGGUCUAGCAAACAUAAU | |
| | GCUAGUCCUAGAUUACUUAUUGAUUUAAAAACAAAAAAACACAA | |
| | AAAAAUAGUAAAAUAUAAAAACAAAUUAAUGUUUUAUAGACCC | |
| | UGGGAAAAAGAAUUUUCAGCAAAGUACAAAAAUUUAAAGCAUU | |
| | CCUUUCUUUAAUUUUGUAAUUCUUUACUGUGGAAUAGCUCAGAA | |
| | UGUCAGUUCUGUUUUAAGUAACAGAAUUGAUAACUGAGCAAGG | |
| | AAACGUAAUUUGGAUUAUAAAAUUCUUGCUUUAAUAAAAAUUC | |
| | CUUAAACAGUG | |
| 54 | UCCCCAGACCUCUGUCCCUGUUCCCCUCCACUCCUCCCCUCACUC | RNA449- |
| | CCCUGCUCCCCCGACCACCUCCUCCUCUGCCUCAAAGACUCUUGU | 3UTR052 |
| | CCUCUUGUCCCUCCUGAGAAAAAAGAAAACGAAAAGUGGGGUUU | |
| | UUUUCUGUUUUCUUUUUUUCCCCUUUCCCCCUGCCCCCACCCACG | |
| | GGGCCUUUUUUUGGAGGUGGGGGCUGGGGAAUGAGGGGCUGAG | |
| | GUCCCGGAAGGGAUUUUAUUUUUUUGAAUUUUAAUUGUAACAU | |
| | UUUUAGAAAAAGAACAAAAAAAGAAAAAAAAAAGAAAGAAACA | |
| | CAGCAACUGUAGAUGCUCCUGUUCCUGGUUCCCGCUUUCCACUU | |
| | CCAAAUCCCUCCCCUCACCUUCCCCCACUGCCCCCCAAGUUCCAG | |
| | GCUCAGUCUUCCAGCCGCCUGGGGAGUCUCUACCUGGGCCCAAG | |
| | CAGGUGUGGGGCCUCCUUCUGGGCUUUUCUUCUGAAUUUAGAGG | |
| | AUUUCUAGAACGUGG | |
| 55 | GCGCAAAGGGGCUGAAAGAACAUGGACUUGUAUAUUUGUACAA | RNA451- |
| | AAAAAAAGUUUUAUUUUUCUAAAAAAAGAAAAAAGAAGAAAAA | 3UTR050 |
| | AUUUAAAGGGUGUACUUAUAUCCACACUGCACACUGCCUGGCCC | |
| | AAAACGUCUUAUUGUGGUAGGAUCAGCCCUCAUUUUGUUGCUUU | |
| | UGUGAACUUUUUGUAGGGGACGAGAAAUGAGGGGCUGAGGUCC | |
| | CGGAAGGGAUUUUAUUUUUUUGAAUUUUAAUUGUAACAUUUUU | |
| | AGAAAAAGAACAAAAAAAGAAAAAAAAAAGAAAGAAACACAGC | |
| | AACUGUAGAUGCUCCUGUUCCUGGUUCCCGCUUUCCACUUCCAA | |
| | AUCC | |
| 56 | UGGAUGUACAUCAUUCCCGUCGUCCUGUUCCUCAUGAUGUCAGG | RNA421- |
| | AGCGCCAGACACCGGGGGCCAGGGUGGGGGUGGGGGUGGGGGUG | 3UTR068 |
| | GUGGUGGGGGUAGUGGCCGGUGAGGGCCCAGGCUGGUCAGCGUC | |
| | CCGUCUUGCACACCCAGGGGCCUCCCUUUCUGCUGGAGUCCCCU | |
| | GUGUCCUCAGCCAUCCCAAGAAGG | |
| 57 | GCAAAACUGCCGCAAGUCUGCAGCCCGGCGCCACCAUCCUGCAG | RNA423- |
| | CCUCCUCCUGACCACGGACGUUUCCAUCAGGUUCCAUCCCGAAA | 3UTR063 |
| | AUCUCUCGGUUCCACGUCCCCCUGGGGCUUCUCCUGACCCAGUCC | |
| | CCGUGCCCCGCCUCCCCGAAACAGGCUACUCUCCUCGGCCCCCUC | |
| | CAUCGGGCUGAGGAAGCACAGCAGCAUCUUCAAACAUGUACAAA | |
| | AUCGAUUGGCUUUAAACACCCUUCACAU | |
| 58 | UGAAAACCAAAAUCCAUGCAAAUGAAAUGUAAUUGGCACGACCC | RNA425- |
| | UCACCCCCAAAUCUUACAUCUCAAUUCCCAUCCUAAAAAGCACU | 3UTR065 |
| | CAUACUUUAUGCAUCCCCGCAGCUACACACACACAACACACAGC | |
| | ACACGCAUGAACACAGCACACACACGAGCACAGCACACACACAA | |
| | ACGCACAGCACACACAGCACACAGAUGAGCACACAGCACACACA | |
| | CAAACGCACAGCACACACACGCACACACAU | |
| 59 | CACACACACGCACACACAUGCACACACAGCACACAAACGCACGGC | RNA427- |
| | ACACACACGCACACACAUGCACACACAGCACACACACAAACGCAC | 3UTR064 |
| | AGCACACACAAACGCACAGCACACACGCACACACAGCACACACAC | |
| | GAGCACACAGCACACAAACGCACAGCACACGCACACACAUGCAC | |
| | ACACAGCACACACACUAGCACACAGCACACACACAAAGACACAG | |
| | CACACACAUGCACACACAGCACACACACGCGAACACAGCACACAC | |
| | GAACACAGCACACACAGCACACACACAAACACAGCACACACAUG | |
| | CACACAGCACACGCACACACAGCACACACAUGAACACAGCACAC | |
| | AGCACACACAUGCACACACAGCACACACGCAUGCACAGCACACA | |
| | UGAACACAGCACACACACAAACACACAGCACACACAUGCACACA | |
| | CAGCACACACACUCAUGCGCAGCACAUACAUGAACACAGCUCAC | |
| | AGCACACAAACACGCAGCACACACGUUGCACACGCAAGCACCCA | |
| | CCUGCACACACACAUGCGCACACACACGCACACCCCCACAAAAUU | |
| | GGAUGAAAACAAUAAGCAUAUCUAAGCAACUACGAUAUCUGUAU | |
| | GGAUCAGGCCAAAGUCCCGCUAAGAUUCUCCAAUGUUUUCAUGG | |
| | UCUGAGCCCCGCUCCUGUUCCCAUCUCCACUGCCCCUCGGCCCUG | |
| | UCUGUGCCCUGCCUCUCAGAGGAGGGGGCUCAGAUGGUGCGGCC | |
| | UGAGUGUGCGGCCGGCGGCAUUUGGGAUACACCCGUAGGGUGGG | |
| | CGGGGUGUGUCCCAGGCCUAAUUCCAUCUUUCCACCAUGACAGA | |
| | GAUGCCCUUGUGAGGCUGGCCUCCUUGGCGCCUGUCCCCACGGC | |
| | CCCCGCAGCGUGAGCCACGAUGCUCCCCAUACCCCACCCAUUCCC | |
| | GAUACACCUUACUUACUGUGUGUUGGCCCAGCCAGAGUGAGGAA | |
| | GGAGUUUGGCCACAUUGGAGAUGGC | |
| 60 | GUCGCAUGAAUGCCAUCUCUGGCUGGCAGGCCUUCUUUCCAGUC | RNA419- |
| | CAUUUCCAGGAGUUCAAUCCUGCCCUGUCACCACAGAGAUCACC | 3UTR067 |
| | CCCAGGGCCCCCGGGGGCUGGCCCUGACCCCCCCUCCCCUCCUGG | |
| | UGCUGACCCCUCCCGGGGGGCUCCUAUAGGGGGGAGAUUUGACC | |
| | GGCAGGCUUCUGCGGAGGGCUGCUUCUACAACGCUGACUACCUG | |
| | GCGGCCCGAGCCCGGCUGGCAGGUGAACUGGCAGGCCAGGAAGA | |
| | GGAGGAAGCCCUGGAGGGGCUGGAGGUGAUGGAUGUUUUCCUCC | |
| | GGUUCUCAGGGCUCCACCUCUUUCGGGCCGUAGAGCCAGGGCUG | |
| | GUGCAGAAGUUCUCCCUGCGAGACUGCAGCCCACGGCUCAGUGA | |
| | AGAACUCUACCACCGCUGCCGCCUCAGCAACCUGGAGGGGCUAG | |
| | GGGGCCGUGCCCAGCUGGCUAUGGCUCUCUUUGAGCAGGAGCAG | |
| | GCCAAUAGCACUUAGCCCGCCUGGGGGCCCUAACCUCAUUACCU | |
| | UUCCUUUGUCUGCCUCAGCCCCAGGAAGGGCAAGGCAAGAUGGU | |
| | GGACAGAUAGAGAAUUGUUGCUGUAUUUUUUAAAUAUGAAAAU | |
| | GUUAUUAAACAUGUCUUCUGCCAAA | |
| 61 | UUCUGGAAUCUGUGCUCUGGGGGCUGUGCCGGGUAGAGAGGGCA | RNA417- |
| | GUGGGAGGUAAGAGCUCUUCACCCUUCACCACCUUCUCCACCCA | 3UTR069 |
| | GCAUGGCCGGCA | |
| 62 | GUUUAGCUUUGUGUUAGCUUAUACAUACUAAAACCUUUAAAAA | RNA413- |
| | GCUUUUCUUCUCAAUUGAUUUUUUUCUUUUAGAAGCCAUGGUGU | 3UTR070 |
| | CUCAACCUUUUGGGGACCUAACUUCUAAACAUUCUAAUAGUUUG | |
| | CCUUAAUUUUUCUUCUGCUUUCUUACUAAAAAUGAAGACAUUCA | |
| | AUACUAAUCUUGCUGGAAGAAGCCUUAACCAAGCAAACUUCUCA | |
| | UUUCUCUGGUGAAAACUGCUGCCAAAACCACUUGUUAAAAAUUG | |
| | UACAGAGCCUGUAGAAAAUAUAGAAGAUUCAUUGGAUGUUGGC | |
| | CUAGUUCUGUGUGGAAGACUAGUGAUUUUGUUGUUUUUAGAUA | |
| | ACUAAAUCGACAACAAAUCACAGUCUGCCAUAUGGCACAGGCCA | |
| | UGCCUCUACAGGACAAAUGAUUGGUGCUGUAAAAUGCAGCAUUU | |
| | CACACCUUACUAGCAUUCUUUGUCUUUUCUACCAAAUAUUAACA | |
| | ACUUUCAAUUCCGUUUUCUUAAUUCUGUUCUACUAAUGUCCGAU | |
| | UUACUACUCAUCAUUUUUCUUGACACUUAACAUUGCUUUAAUUU | |
| | GUAAUUGCUAAUGGUUUUUGAACUCUUCUAAUUGUAAUGGACG | |
| | UGUUUAUCAUUUUAAUUUAGCAUUGAAAUUGUCUUGAUGUUGA | |
| | U | |
| 63 | CCUGAGGCUGGGUUGGAGCAGCCCUCCUGUGCCUGAGGCCAGCU | RNA415- |
| | CCCAGGCCCUUGGAUCACCGCGGGAGGAACCCUCAGGAUGGGUG | 3UTR066 |
| | GAGCCUCCAGGCUAUGGGCAUUGCCUGCCUGAUGCCAGCACCAC | |
| | CUGGGCUGGGCCCUGGGCUUGGCUCGAGUUCUCCUGCUGGUGAG | |
| | GCUCCGGAUCUCAGGAGCAGCCCUGAGUCUGCUUCCCAGGCUGC | |
| | CCCUGCCAGGCCUGCAGCCUCCCCAGCCAGGGCUGCUCUCUGCUG | |
| | UCCCCAUUCAGUGCCCUGGCCCCUGCAUUCAUGCCCCCCACACCC | |
| | CCUCAGGCCCUGUGCCUGGACUUUGGGGCUGGCAGCUGAAGCCU | |
| | UGAGAUCCUGGGCCAGCUGCCGGCACACAGCUAGGCAGACUCUC | |
| | CCACCAGGUGCCCCUGCCCAGGCCUCCUAAUCGGGGGCAGACAG | |
| | GCAGGGAGGGUGUGGCUGGGCUGGGCUGGGCGGGGCGGCCUGGG | |
| | GCAGGGGUGUGGCCCCUAAAUGUCCCCAACCUCAGAGGGACCUA | |
| | GAGUCCUGAGCCUCCAGUAGCUUCUCUGGGCCUGGCAGAGGUAA | |
| | GGGGGAGGCAACCCUGGAGUGUCUGGAGGCCCAUGGCUGGCUGA | |
| | ACCCUGGAUGCCUUUUCUUCCGCGUCCCCAUGAAUGAAAGCUGU | |
| | CUGGGCCUUCAUUCUGCAGACAGGGACAAACAGCUCCAUGCUGU | |
| | UUGUCCUCCCAGUGCAGCCGUGCUGGGAGGGUCUGGGGGAGCUU | |
| | CCUACAAGGAGAGACUCCUGCUGCUUUGGAAAACUGAGAAAAAA | |
| | UAGGGGUCUAACCCUCUCCUCCCAUUUUACAAGUGGGGAAAUGA | |
| | GGCGUGAAAGGAGAGGCGUCUGGGUUACUCCGUGGGUCUGGGGU | |
| | CCAGGGAAGGGCCUGUAUGGGGGAGGGAGCUGGGAGGGGACGG | |
| | UGUCUGGCUCUACCCCUGUGGGGUGGGGAGGUGGGGCUCCCCUG | |
| | UAUCACAGGACAUCCCCCCUGAGAGGUCCCUCAUAUGUCUGGGU | |
| | CCUGUGGGUGGGGGACUAACUGCGCAAUGUAGUUAGGUGCUCAA | |
| | UAAACGGAGUUGCCGCUGA | |
| 64 | UGUUCAGAGGUCCCUGUCUUCUGUCCCCAUCUUCCUGCCGAUAG | RNA433- |
| | CUAUCCCCUGUAUGAUGUUGGAUGCUCCUCACAUGCUGAGUUUC | 3UTR059 |
| | CAGCCUUUUCUGAAACUCAGUAGCUGGGGAGAGGGCAGGGAGGC | |
| | UUCCUGGGCCUUCCAGCCUCCUUCCCCACCUCCUUCCCAAACCCU | |
| | CUUGGGAACUCCUCAGGGACAACUACUGCUGAGUUUGGGUGCAC | |
| | CCUAAGAUGGAGGCCAGGUAGCAAUGGGGCCGGCCUCAGAGAGA | |
| | GCGCUGUGUGUGUGUGUGUGUGUGUGUGUGUGUGUGUGUGUCG | |
| | GCCUCAGAGCGCGCUGUGUGUGUGUGUGUGUGUGUCAGCCUCAG | |
| | AGCGCGCGCUGUGUGUGUGUGUGUGUGUGCGUGCUUGUGACCCU | |
| | GUAUUGUUUGAUAGGAUCCAUUCAGUUUCCCCAAGUACCUGUUU | |
| | UCAUUCCCAUUUUUCCCAUUGUUUAAAACCAUCACUUUUUUGUC | |
| | UUUGGGAAACCACAGG | |
| 65 | CUUUUCACAAGAUGGACCCUUCAUUUCAAGCUUAGGCUGGCGUU | RNA429- |
| | ACUUUUGCUGUCUAGUCAGGACUAAUCACGGUGUUUCAGUGCGG | 3UTR061 |
| | AGUGCCAAGAGUCCUAUCCUGACGUCAGGCUCUGGGUGUCAACC | |
| | UCUGACUUAUUCUGCAGAUGCUCUGUGUGUGUGUGUGUGUGUG | |
| | UGUGUGUGUGUGUGUGUGUGUGUGUGUUCGGGGAGAGGGUGGU | |
| | AGCACAGGGCUUGGGAUAUCGGCAGUGUGGGAAAUGCGAAGCAU | |
| | UUCUCAUCAUCAUCAUCUCUGCUACAGUCAUGUUUCUGCAUGUC | |
| | AGCGAGCGACACUGUCCCUGCCUCAGGUUGGAGGUUUUAUCAGC | |
| | CAAAGUGUUUUUUUCAUGUAUCGUUCGUUCCAUUCAUCCACUCU | |
| | GUGCCUUGUCAGCCUUUGAAAGGCUUGGUUGCUCCCAGGCUGCU | |
| | GUUCUCAGGGACCUUAAAAGGGACCUGGUUAGUCUUGGGGCAGA | |
| | GAGUAUCUACUUGGGCACCUUUUCACAAGAUGGACCCUUCAUUU | |
| | CAAGCUUAGGCUGGCGUUACUUUUGCUGUCUAGUCAGGACUAAU | |
| | CACGGUGUUUCAGUGCGGAGUGCCAAGAGUCCUAUCCUGACGUC | |
| | AGGCUCUGGGUGUCAACCUCUGACUUAUUCUGCAGAUGCUCUGU | |
| | GUGUGUGUGUGUGUGUGUGUGUGUGUGUGUGUGUGUGUGUGUG | |
| | UUCGGGGAGAGGGUGGUAGCACAGGGCUUGGGAUAUCGGCAGU | |
| | GUGGGAAAUGCGAAGCAUUUCUCAUCAUCAUCAUCUCUGCUACA | |
| | GUCAUGUUUCUGCAUGUCAGCGAGCGACACUGUCCCUGCCUCAG | |
| | GUUGGAGGUUUUAUCAGCCAAAGUGUUUUUUUCAUGUAUCGUU | |
| | CGUUCCAUUCAUCCACUCUGUGCCUUGUCAGCCUUUGAAAGGCU | |
| | UGGUUGCUCCCAGGCUGCUGUUCUCAGGGACCUUAAAAGGGACC | |
| | UGGUUAGUCUUGGGGCAGAGAGUAUCUACUUGGGCAC | |
| 66 | GUGUGUGUACAUCUGGGUGUGUGUGAGCAAGUGUGCAGAGGGU | RNA431- |
| | GCGUGUGCACAGGUACGGGCAGGUGCAUGUGUGCGAGUGGCUGU | 3UTR060 |
| | GUGAGUGGGUGUGUGAGAAGGUGAAUGUGAGAGCUGGUGUGGC | |
| | UGAGAGUGGGUGUGGCCAUGUUUGUGUGUCUGUCAACACAUGA | |
| | AUGUAUUUCAGUGCGUGUGAGCAGCUGUGCACGUAUGAUGUCAG | |
| | UGGGUGUCAGCAGUGUGUGGGUGUGCCUGUGUGAGCAGGGAUG | |
| | UGAGCAUGGUAUAUGUGAGUGGCUGUAAGCAGGCAUGUGUGAG | |
| | UGAUAUGUGUGAGUGGGGGUGUGACAGCAGGUGUGUGAGCAAG | |
| | UGUAUAUGUGUGAAUGUGUAUGUAAGCAGGGAUGUGUGUGAUA | |
| | UAAGUGUGUGUGAAUGGGCAUAGGUGUGU | |
| 67 | CUUUUCACAAGAUGGACCCUUCAUUUCAAGCUUAGGCUGGCGUU | RNA435- |
| | ACUUUUGCUGUCUAGUCAGGACUAAUCACGGUGUUUCAGUGCGG | 3UTR062 |
| | AGUGCCAAGAGUCCUAUCCUGACGUCAGGCUCUGGGUGUCAACC | |
| | UCUGACUUAUUCUGCAGAUGCUCUGUGUGUGUGUGUGUGUGUG | |
| | UGUGUGUGUGUGUGUGUGUGUGUGUGUUCGGGGAGAGGGUGGU | |
| | AGCACAGGGCUUGGGAUAUCGGCAGUGUGGGAAAUGCGAAGCAU | |
| | UUCUCAUCAUCAUCAUCUCUGCUACAGUCAUGUUUCUGCAUGUC | |
| | AGCGAGCGACACUGUCCCUGCCUCAGGUUGGAGGUUUUAUCAGC | |
| | CAAAGUGUUUUUUUCAUGUAUCGUUCGUUCCAUUCAUCCACUCU | |
| | GUGCCUUGUCAGCCUUUGAAAGGCUUGGUUGCUCCCAGGCUGCU | |
| | GUUCUCAGGGACCUUAAAAGGGACCUGGUUAGUCUUGGGGCAGA | |
| | GAGUAUCUACUUGGGCAC | |
| 68 | CUAGGUGCAGAGAGCCCAGGCCUUAUGUUAAAAUCAUGCACUUG | RNA439- |
| | AAAAGCAAACCUUAAUCUGCAAAGACAGCAGCAAGCAUUAUACG | 3UTR058 |
| | GUCAUCUUGAAUGAUCCCUUUGAAAUUUUUUUUUUGUUUGUUU | |
| | GUUUAAAUCAAGCCUGAGGCUGGUGAACAGUAGCUACACACCCA | |
| | UAUUGUGUGUUCUGUGAAUGCUAGCUCUCUUGAAUUUGGAUAU | |
| | UGGUUAUUUUUUAUAGAGUGUAAACCAAGUUUUAUAUUCUGCA | |
| | AUGCGAACAGGUACCUAUCUGUUUCUAAAUAAAACUGUUUACAU | |
| | UCAUUAUGGGGUAUGUAUGACCUUCAUUUUCCAAGAAAUAGAAC | |
| | UCUAGCUUAGAAUUAUGGAUGCUCUAAAAUGUCAGAAUGGGAA | |
| | CUCUCCUCGAAGUUCUCCCAAACUCAGAGACAGCACUGCCUUCU | |
| | CCUAAAUGAUUAUUCUUUUCUCCCUGUUUUCUGGUAUUUUCUAG | |
| | CAUCCUUCUCACCACAGCCA | |
| 69 | AGGGAGCCAUGAGGGUCUGGGCUUCAGAGCUAGGUCUUUGGGGA | RNA441- |
| | AGUCCUGGCUGACUGCCUUAGCAGUGGGGGUGGGGGUGGGGGCA | 3UTR056 |
| | GGGGCAGGGGCUUUAUGUGUUUUUGCUUGGGGGGCGCUGGGCCU | |
| | AGCCCAGAGUAGUGCUUGCUCCCCCUGCCUUGUCCCACCAGGGA | |
| | GGCAGCAGACUCAGGCCCUCCAUGGUCCUCUUUGUCAUUUUGUU | |
| | GACAUGCAUUCCUCCUUUUGUCAUCUUGUUGGGGGGAGGGGAUU | |
| | AACCAAAGGCCACCCUGACUUUGUUUUUGUGGACACACAAUAAA | |
| | AGCCCCGUUUAUUUGUAA | |
| 70 | ACACACACACAACACACAGCACACGCAUGAACACAGCACACACA | RNA443- |
| | CGAGCACAGCACACACACAAACGCACAGCACACACAGCACACAG | 3UTR053 |
| | AUGAGCACACAGCACACACACAAACGCACAGCACACACACGCAC | |
| | ACACAUGCACACACAGCACACAAACGCACGGCACACACACGCAC | |
| | ACACAUGCACACACAGCACACACACAAACGCACAGCACACACAA | |
| | ACGCACAGCACACACGCACACACAGCACACACACGAGCACACAGC | |
| | ACACAAACGCACAGCACACGCACACACAUGCACACACAGCACAC | |
| | ACACUAGCACACAGCACACACACAAAGACACAGCACACACAUGC | |
| | ACACACAGCACACACACGCGAACACAGCACACACGAACACAGCA | |
| | CACACAGCACACACACAAACACAGCACACACAUGCACACAGCAC | |
| | ACGCACACACAGCACACACAUGAACACAGCACACAGCACACACA | |
| | UGCACACACAGCACACACGCAUGCACAGCACACAUGAACACAGC | |
| | ACACACACAAACACACAGCACACACAUGCACACACAGCACACAC | |
| | ACUCAUGCGCAGCACAUACAUGAACACAGCUCACAGCACACAAA | |
| | CACGCAGCACACACGUUGCACACGCAAGCACCCACCUGCACACAC | |
| | ACAUGCGCACACACACGCACACCCCCACAAAAUUGGAUGAAAAC | |
| | AAUAAGCAUAUCUAAGCAACUACGAUAUCUGUAUGGAUCAGGCC | |
| | AAAGUCCCGCUAAGAUUCUCCAAUGUUUUCAUGGUCUGAGCCCC | |
| | GCUCCUGUUCCCAUCUCCACUGCCCCUCGGCCCUGUCUGUGCCCU | |
| | GCCUCUCAGAGGAGGGGGCUCAGAUGGUGCGGCCUGAGUGUGCG | |
| | GCCGGCGGCAUUUGGGAUACACCCGUAGGGUGGGGGGGUGUGU | |
| | CCCAGGCCUAAUUCCAUCUUUCCACCAUGACAGAGAUGCCCUUG | |
| | UGAGGCUGGCCUCCUUGGCGCCUGUCCCC | |
| 71 | GGGUUGCAGACACAUAUAUUUUUGAGGCUGGGUGACGAGAAAA | RNA437- |
| | UCUAGAGACAUGAGGGACAUAAAUGGGCCUGGCAGCCUCGGCUC | 3UTR057 |
| | UUUGCGGCUGCUGGCAGGACUGAGCUGUCCGGGUUCUCCCCACA | |
| | CUUCCAGCACAGCUGUGCUCUGUGUCCUGCCUCGGCGCUCUCGC | |
| | AAAUGAAGCUGCAGGCCAAGAA | |
| 72 | GGGUGUGCCUCCACAAGUGUGCAAGGGUAUAUGUAUAUAUUUGC | RNA455- |
| | AGGCAUGUGUGAGGCGCACUAGUAAGUACAUAAUUUGCUGGCAU | 3UTR051 |
| | GAUUGCAGGUAGGCAUGAGUGUGUAUGUGCUAACAUGUAGGCU | |
| | GGUGUGUGUAGGCAUGUGUGAAUAAGCAUGUAUAAAUGAGCUU | |
| | AAGUGUGCUGUGUGCACAUGUGUACACACAGCUAAUUUAUCUGC | |
| | AGACCUAUAUGUGAGCAUGUAAGAGUGAACAUAUGUGUGUGUG | |
| | UGUAGUAUGUAAGAAUGAGGAACUCGGUAUGUGCAUGUGUAGA | |
| | CAGGUACCCAGCAGUGUGUGUAUAUGCGCAUGAGUGAGGAUGUA | |
| | UAGGCAGAAGAUGUGUGUGUCUAUGAGUGAGUUUGAGUGUGCA | |
| | GGCUUGUAUUAGGUGUAUGUGAGGAAGCCCUUGUGUGUGCAGG | |
| | GGUGCACAUGUUUGGCCACAGGCAUGGGAGGUGUAUGUUAGGA | |
| | GCAUGUGUGUUUGUAGGCAGACUCAUGAGCAGGUGUGUGCAAA | |
| | UACAUAUCCAGCUGCACUGUGGGUGUCCACCCACACCUUGUGUU | |
| | CCUCAUGGCCUACCCCAGCCUUUCUUCUCCACUGGGUCCCACUGU | |
| | UCCCUGGAGACAGAGGGCUAGCAUGCUGUCAUUUAUCUGAA | |
| 73 | AUGGCUAUCCUUUAAUGAUGCGUGUGGAAUGUGUGUGUGUGCU | RNA457- |
| | CAGGCAAUUAUUUUGCUAAGAAUGUGAAUUCAAGUGCAGCUCAA | 3UTR047 |
| | UACUAGCUUCAGUAUAAAAACUGUACAGAUUUUUGUAUAGCUG | |
| | AUAAGAUUCUCUGUAGAGAAAAUACUUUUAAAAAAUGCAGGUU | |
| | GUAGCUUUUUGAUGGGCUACUCAUACAGUUAGAUUUUACAGCUU | |
| | CUGAUGUUGAAUGUUCCUAAAUAUUUAAUGGUUUUUUUAAUUU | |
| | CUUGUGUAUGGUAGCACAGCAAACUUGUAGGAAUUAGUAUCAA | |
| | UAGUAAAUUUUGGGUUUUUUAGGAUGUUGCAUUUCGUUUUUUU | |
| | AAAAAAAAUUUUGUAAUAAAAUUAUGUAUAUUAUUUCUAUUGU | |
| | CUUUGUCUUAAUAUGCUAAGUUAAUUUUCACUUUAAAAAAGCCA | |
| | UUUGAAGACCAGAGCUAUGUUGAUUUUUUUCGGUAUUUCUGCCU | |
| | AGUAGUUCUUAGACACAGUUGAC | |
| 74 | CAACUCAGCUCACAUCACCAGCUCACCUCUGGUAGCCAUAGCAG | RNA453- |
| | CCCCUGCUUCAGCCCCACCGCACCCCUCCAGGGGGCCUGCCUUUC | 3UTR049 |
| | CCUGACACUUUUGGGGUCUGCCUGGGGGAGGAGGGGAGAAAGCA | |
| | CCAUGAGUGCUCACUAAAACAACUUUUUCCAUUUUUAAUAAAAC | |
| | GCCAAAAAUAUCACAACCCACCAAAAAUAGAUGCCUCUCCCCCU | |
| | CCAGCCCUAGCCGAGCUGGUCCUAGGCCCCGCCUAGUGCCCCACC | |
| | CCCACCCACAGUGCUGCACUCCUCCUGCCCCUGCCACGCCCACCC | |
| | CCUGCCCACCUCUCCAGGCUCUGCUCUGCAGCACACCCGUGGGUG | |
| | ACCCCUCACCCCAGAAGCAGCAGUGGCAGCUUGGGAAAUGUGAG | |
| | GAAGGGAAGGAGGGAGAGACGGGAGGGAGGAGAGAGAGGAGAA | |
| | GGGAGGCAGGGGAGGGGCAGCAGAACCAAGGCAAAUAUUUCAGC | |
| | UGGGCUAUACCCCUCUCCCCAUCCCUGUUAUAGAAGCUUAGAGA | |
| | GCCAGCCAGCAAUGGAACCUUCUGGUUCCUGCGCCAAUCGCCAC | |
| | CAGUAUCAAUUGUGUGAGCUUGGGUGCGAGUGCACGCGUGCGUG | |
| | AGUACGGAGAGUAUAUAUAGAUCUCUAUCUCUUAGCAAAGGUG | |
| | AAUGCCAGAUGUAAAUGGCGCCUCUGGGCAAAGGAGGCUUGUAU | |
| | UUUGCACAUUUUAUAAAAACUUGAGAGAAUGAGAUUUCUGCUU | |
| | GUAUAUUUCUAAAAAGAGGAAGGAGCCCAAACCAUCCUCUCCUU | |
| | ACCACUCCCAUCCCUGUGAGCCCUACCUUACCCCUCUGCCCCUAG | |
| | CCAAGGAGUGUGAAUUUAUAGAUCUAACUUUCAUAGGCAAAACA | |
| | AAAGCUUCGAGCUGUUGCGUGUGUGAGUCUGUUGUGUGGAUGU | |
| | GCGUGUGUGGUCCCCAGCCCCAGACUGGAUUGGAAAAGUGCAUG | |
| | GUGGGGGCCUCGGGGCUGUCCCCACGCUGUCCCUUUGCCACAAG | |
| | UCUGUGGGGCAAGAGGCUGCAAUAUUCCGUCCUGGGUGUCUGGG | |
| | CUGCUAACCUGGCCUGCUCAGGCUUCCCACCCUGUGCGGGGCAC | |
| | ACCCCCAGGAAGGGACCCUGGACACGGCUCCCACGUCCAGGCUU | |
| | AAGGUGGAUGCACUUCCCGCACCUCCAGUCUUCUGUGUAGCAGC | |
| | UUUAACCCACGUUUGUCUGUCACGUCCAGUCCCGAGACGGCUGA | |
| | GUGACCCCAAGAAAGGCUUCCCCGACACCCAGACAGAGGCUGCA | |
| | GGGCUGGGGCUGGGUGAGGGUGGCGGGCCUGCGGGGACAUUCUA | |
| | CUGUGCUAAAAAGCCACUGCAGACAUAGCAAUAAAAACAUGUCA | |
| | UUUUCCAAA | |
| 75 | GGGUGUGCCUCCACAAGUGUGCAAGGGUAUAUGUAUAUAUUUGC | RNA459- |
| | AGGCAUGUGUGAGGCGCACUAGUAAGUACAUAAUUUGCUGGCAU | 3UTR048 |
| | GAUUGCAGGUAGGCAUGAGUGUGUAUGUGCUAACAUGUAGGCU | |
| | GGUGUGUGUAGGCAUGUGUGAAUAAGCAUGUAUAAAUGAGCUU | |
| | AAGUGUGCUGUGUGCACAUGUGUACACACAGCUAAUUUAUCUGC | |
| | AGACCUAUAUGUGAGCAUGUAAGAGUGAACAUAUGUGUGUGUG | |
| | UGUAGUAUGUAAGAAUGAGGAACUCGGUAUGUGCAUGUGUAGA | |
| | CAGGUACCCAGCAGUGUGUGUAUAUGCGCAUGAGUGAGGAUGUA | |
| | UAGGCAGAAGAUGUGUGUGUCUAUGAGUGAGUUUGAGUGUGCA | |
| | GGCUUGUAUUAGGUGUAUGUGAGGAAGCCCUUGUGUGUGCAGG | |
| | GGUGCACAUGUUUGGCCACAGGCAUGGGAGGUGUAUGUUAGGA | |
| | GCAUGUGUGUUUGUAGGCAGACUCAUGAGCAGGUGUGUGCAAA | |
| | UACAUAUCCAGCUGCACUGUGGGUGUCCACCCACACCUUGUGUU | |
| | CCUCAUGGCCUACCCCAGCCUUUCUUCUCCACUGGGUCCCACUGU | |
| | UCCCUGGAGACAGAGGGCUAGCAUGCUGUCAUUUAUCUGAAUUC | |
| | UGGUUCCUGCGCCAAUCGCCACCAGUAUCAAUUGUGUGAGCUUG | |
| | GGUGCGAGUGCACGCGUGCGUGAGUACGGAGAGUAUAUAUAGA | |
| | UCUCUAUCUCUUAGCAAAGGUGAAUGCCAGAUGUAAAUGGCGCC | |
| | UCUGGGCAAAGGAGGCUUGUAUUUUGCACAUUUUAUAAAAACU | |
| | UGAGAGAAUGAGAUUUCUGCUUGUAUAUUUCUAAAAAGAGGAA | |
| | GGAGCCCAAACCAUCCUCUCCUUACCACUCCCAUCCCUGUGAGCC | |
| | CUACCUUACCCCUCUGCCCCUAGCCAAGGAGUGUGAAUUUAUAG | |
| | AUCUAACUUUCAUAGGCAAAACAAAAGCUUCGAGCUGUUGCGUG | |
| | UGUGAGUCUGUUGUGUGGAUGUGCGUGUGUGGUCCCCAGCCCCA | |
| | GACUGGAUUGGAAAAGUGCAUGGUGGGGGCCUCGGGGCUGUCCC | |
| | CACGCUGUCCCUUUGCCACAAGUCUGUGGGGCAAGAGGCUGCAA | |
| | UAUUCCGUCCUGGGU | |
| 76 | CMAWURUKTU | oncoselective |
| | | sequence |
| | | motif |
| | | 1 |
| 77 | GCUAAGAAU | oncoselective |
| | | sequence |
| | | motif |
| | | 2 |
| 78 | CUGUA | oncoselective |
| | | sequence |
| | | motif |
| | | 3 |
| 79 | AGVURM | oncoselective |
| | | sequence |
| | | motif |
| | | 4 |
| 80 | YUGUA | oncoselective |
| | | sequence |
| | | motif |
| | | 5 |
| 81 | GUGAAYUCAAGUGCAGCUCAWURUUAGCUUCAGUAUAAAAA | oncoselective |
| | | sequence |
| | | motif |
| | | 6 |
| 82 | GUGMAYUCAW | oncoselective |
| | | sequence |
| | | motif |
| | | 7 |
| 83 | GUGMAGYUCAW | oncoselective |
| | | sequence |
| | | motif |
| | | 8 |
| 84 | CMAWURUKU | oncoselective |
| | | sequence |
| | | motif |
| | | 9 |
| 85 | UGRMUNUC | oncoselective |
| | | sequence |
| | | motif |
| | | 10 |
| 86 | CUGYWRURADGYK | oncoselective |
| | | sequence |
| | | motif |
| | | 11 |
| 87 | UGUGSWN | oncoselective |
| | | sequence |
| | | motif |
| | | 12 |
| 88 | URUGYG | oncoselective |
| | | sequence |
| | | motif |
| | | 13 |
| 89 | AYNBUCC | oncoselective |
| | | sequence |
| | | motif |
| | | 14 |
| 90 | AYNBUCC | Oncoselective |
| | | sequence |
| | | motif |
| | | 15 |
| 91 | AGGGTCTGAGCGCCCGGCGGAAAACCGAAGTTGGAAGTGTCTCTTAGCA | 5UTR134 |
| | GCGCGCGGAGAAGAACGGGGAGCCAGCATCCCGCCACC | |
| 92 | AGGGACTTCGAGAGGGACTTAGAGAAGGCAGACGCATCCCGAACTCGCT | SUTR136 |
| | GGAGGACAAGGCTCAGCTCTTGCCAGGCCAAATTGAGACCCGCCACC | |
| 93 | AGGGTGCCTCTGTTTGGCGCTTTTGTGCGCGCCCGGGTCTGTTGGTGCTC | 5UTR137 |
| | AGAGTGTGGTCAGGCGGCTCGGACTGAGCAGGACTTTCCTTATCCCAGTT | |
| | GATTGTGCAGAATACACTGCCTGTCGCTTGTCTTCTATTCACCCCGCCAC | |
| | C | |
| 94 | TTAAGCTGCCTTCTGCGGGGCTTGCCTTCTGGCCATGCCCTTCTTCT | 3UTR002 |
| | CTCCCTTGCACCTGTACCTCTTGGTCTTTGAATAAAGCCTGAGTAG | |
| | GAAGAAGCCTGCATGCCTGGTTCTCTGCGTCTGC | |
| 95 | CACCACGTGCCAGAGCCGCCCACCCGGAGCCGCCCGCATGCAGCTTCAC | 3UTR317 |
| | CTCCCCTTTCCAGGCGCCACTGTTGAGAAGCTAGAGATTGTATGAGAATA | |
| | AACTTGTTATTATGGAAGCCTGGCTCCCACCCCATCTA | |
| 96 | ATTTTAGGTATCTGCCTTGGTTTCAGTGGGGACATCTGGGGCTTATGGGG | 3UTR318 |
| | CTGGGATGAGGAGCTGGATGATTCTAGGAAGGCCCAAGTTGGAGAATGA | |
| | TGTGGAGAGTGTGCCAAGACACTGCTTTTGGCATTTTATTCCTTTCTGTTT | |
| | GCTGGATGTCAATTGACCCTTTTATTTTCTCTTACTTGTGTTTTCAGATATT | |
| | GTTAATCCTGCCAGTCTTTCTCTTCAAGCCAGGGTGCATCCTCAGAAACCT | |
| | ACTCAACACAGCACTCTAGGCAGCCACTATCAATCAATTGAAGTTGACAC | |
| | TCTGCATTAAATCTATTTGCC | |
| 97 | TACTTAATAAATATTTGCTGTTGATGATAGCAATGACCTTGAGACTGATG | 3UTR319 |
| | AACAGTCTGGCCAAGAGGATCCTTGATGTGGAAGATAGAAAAAGCCTTT | |
| | GGGGTCAGGCAGACTTGGATTCTAATACCAGCCAGTTCTGCTTGCTGTGT | |
| | CTGAGCCTCAGTTTACTCATCTGTGAAGAGGAGGTAGCAAGAATGAAAA | |
| | TGCCTGCCTTGTGGTTTGTTGTAAGGACAGACACTGCCAACGTAGAGGGC | |
| | CCAGCAGCTCACAGACCAGTTGCTCTGAGAGCAG | |
| 3′- | | |
| UTR | | |
| Combi- | | |
| nations | | |
| SEQ ID | GCTCTTTGGAGCACAGAGAACTTTGATTTTTAAGCTTCTACAGATA | 3UTR481; |
| NO: | TGGTCCTAGAGACTGGGGTCCTCAGACACTCCCGCTGTATGTGGTG | 3UTR325-L- |
| 101 | AGCTTGGCTGGAAACTTGCCATAGCCAGGACTGAGGGGCTGGCCC | 3UTR318 |
| | CAGGACGCTCTCAAGAAATGGGGCTACTGTGGTTTAGACACTCCTG | |
| | CTGCTCACATTGATGGGTGGCTATTAAAGAAAACAACAACAACAA | |
| | CAACTCCAACTCACATTTTAGGTATCTGCCTTGGTTTCAGTGGGGA | |
| | CATCTGGGGCTTATGGGGCTGGGATGAGGAGCTGGATGATTCTAG | |
| | GAAGGCCCAAGTTGGAGAATGATGTGGAGAGTGTGCCAAGACACT | |
| | GCTTTTGGCATTTTATTCCTTTCTGTTTGCTGGATGTCAATTGACCC | |
| | TTTTATTTTCTCTTACTTGTGTTTTCAGATATTGTTAATCCTGCCAGT | |
| | CTTTCTCTTCAAGCCAGGGTGCATCCTCAGAAACCTACTCAACACA | |
| | GCACTCTAGGCAGCCACTATCAATCAATTGAAGTTGACACTCTGCA | |
| | TTAAATCTATTTGCC | |
| SEQ ID | GCTCTTTGGAGCACAGAGAACTTTGATTTTTAAGCTTCTACAGATA | 3UTR485; |
| NO: | TGGTCCTAGAGACTGGGGTCCTCAGACACTCCCGCTGTATGTGGTG | 3UTR343-L- |
| 102 | AGCTTGGCTGGAAACTTGCCATAGCCAGGACTGAGGGGCTGGCCC | 3UTR318 |
| | CAGGACGCTCTCAAGAAATGGGGCTACTGTGGTTTAGACACTCCA | |
| | AAACAACAACAACAACAACTCCAACTCACATTTTAGGTATCTGCCT | |
| | TGGTTTCAGTGGGGACATCTGGGGCTTATGGGGCTGGGATGAGGA | |
| | GCTGGATGATTCTAGGAAGGCCCAAGTTGGAGAATGATGTGGAGA | |
| | GTGTGCCAAGACACTGCTTTTGGCATTTTATTCCTTTCTGTTTGCTG | |
| | GATGTCAATTGACCCTTTTATTTTCTCTTACTTGTGTTTTCAGATAT | |
| | TGTTAATCCTGCCAGTCTTTCTCTTCAAGCCAGGGTGCATCCTCAG | |
| | AAACCTACTCAACACAGCACTCTAGGCAGCCACTATCAATCAATTG | |
| | AAGTTGACACTCTGCATTAAATCTATTTGCC | |
| SEQ ID | GCTCTTTGGAGCACAGAGAACTTTGATTTTTAAGCTTCTACAGATA | 3UTR480; |
| NO: | TGGTCCTAGAGACTGGGGTCCTCAGACACTCCCGCTGTATGTGGTG | 3UTR325-L- |
| 103 | AGCTTGGCTGGAAACTTGCCATAGCCAGGACTGAGGGGCTGGCCC | 3UTR319 |
| | CAGGACGCTCTCAAGAAATGGGGCTACTGTGGTTTAGACACTCCTG | |
| | CTGCTCACATTGATGGGTGGCTATTAAAGAAAACAACAACAACAA | |
| | CAACTCCAACTCACTACTTAATAAATATTTGCTGTTGATGATAGCA | |
| | ATGACCTTGAGACTGATGAACAGTCTGGCCAAGAGGATCCTTGAT | |
| | GTGGAAGATAGAAAAAGCCTTTGGGGTCAGGCAGACTTGGATTCT | |
| | AATACCAGCCAGTTCTGCTTGCTGTGTCTGAGCCTCAGTTTACTCA | |
| | TCTGTGAAGAGGAGGTAGCAAGAATGAAAATGCCTGCCTTGTGGT | |
| | TTGTTGTAAGGACAGACACTGCCAACGTAGAGGGCCCAGCAGCTC | |
| | ACAGACCAGTTGCTCTGAGAGCAG | |
| SEQ ID | GCTCTTTGGAGCACAGAGAACTTTGATTTTTAAGCTTCTACAGATA | 3UTR484; |
| NO: | TGGTCCTAGAGACTGGGGTCCTCAGACACTCCCGCTGTATGTGGTG | 3UTR343-L- |
| 104 | AGCTTGGCTGGAAACTTGCCATAGCCAGGACTGAGGGGCTGGCCC | 3UTR319 |
| | CAGGACGCTCTCAAGAAATGGGGCTACTGTGGTTTAGACACTCCA | |
| | AAACAACAACAACAACAACTCCAACTCACTACTTAATAAATATTT | |
| | GCTGTTGATGATAGCAATGACCTTGAGACTGATGAACAGTCTGGCC | |
| | AAGAGGATCCTTGATGTGGAAGATAGAAAAAGCCTTTGGGGTCAG | |
| | GCAGACTTGGATTCTAATACCAGCCAGTTCTGCTTGCTGTGTCTGA | |
| | GCCTCAGTTTACTCATCTGTGAAGAGGAGGTAGCAAGAATGAAAA | |
| | TGCCTGCCTTGTGGTTTGTTGTAAGGACAGACACTGCCAACGTAGA | |
| | GGGCCCAGCAGCTCACAGACCAGTTGCTCTGAGAGCAG | |
| SEQ ID | ATTTTAGGTATCTGCCTTGGTTTCAGTGGGGACATCTGGGGCTTAT | 3UTR478; |
| NO: | GGGGCTGGGATGAGGAGCTGGATGATTCTAGGAAGGCCCAAGTTG | 3UTR318-L- |
| 105 | GAGAATGATGTGGAGAGTGTGCCAAGACACTGCTTTTGGCATTTTA | 3UTR325 |
| | TTCCTTTCTGTTTGCTGGATGTCAATTGACCCTTTTATTTTCTCTTAC | |
| | TTGTGTTTTCAGATATTGTTAATCCTGCCAGTCTTTCTCTTCAAGCC | |
| | AGGGTGCATCCTCAGAAACCTACTCAACACAGCACTCTAGGCAGC | |
| | CACTATCAATCAATTGAAGTTGACACTCTGCATTAAATCTATTTGC | |
| | CAAAACAACAACAACAACAACTCCAACTCACGCTCTTTGGAGCAC | |
| | AGAGAACTTTGATTTTTAAGCTTCTACAGATATGGTCCTAGAGACT | |
| | GGGGTCCTCAGACACTCCCGCTGTATGTGGTGAGCTTGGCTGGAAA | |
| | CTTGCCATAGCCAGGACTGAGGGGCTGGCCCCAGGACGCTCTCAA | |
| | GAAATGGGGCTACTGTGGTTTAGACACTCCTGCTGCTCACATTGAT | |
| | GGGTGGCTATTAAAG | |
| SEQ ID | ATTTTAGGTATCTGCCTTGGTTTCAGTGGGGACATCTGGGGCTTAT | 3UTR490; |
| NO: | GGGGCTGGGATGAGGAGCTGGATGATTCTAGGAAGGCCCAAGTTG | 3UTR318-L- |
| 106 | GAGAATGATGTGGAGAGTGTGCCAAGACACTGCTTTTGGCATTTTA | 3UTR488 |
| | TTCCTTTCTGTTTGCTGGATGTCAATTGACCCTTTTATTTTCTCTTAC | |
| | TTGTGTTTTCAGATATTGTTAATCCTGCCAGTCTTTCTCTTCAAGCC | |
| | AGGGTGCATCCTCAGAAACCTACTCAACACAGCACTCTAGGCAGC | |
| | CACTATCAATCAATTGAAGTTGACACTCTGCATTAAATCTATTTGC | |
| | CAAAACAACAACAACAACAACTCCAACTCACCTTTGATTTTTAAGC | |
| | TTCTACAGATATGGTCCTAGAGACTGGGGTCCTCAGACACTCCCGC | |
| | TGTATGTGGTGAGCTTGGCTGGAAACTTGCCATAGCCAGGACTGA | |
| | GGGGCTGGCCCCAGGACGCTCTCAAGAAATGGGGCTACTGTGGTT | |
| | TAGACACTCC | |
| SEQ ID | TACTTAATAAATATTTGCTGTTGATGATAGCAATGACCTTGAGACT | 3UTR476; |
| NO: | GATGAACAGTCTGGCCAAGAGGATCCTTGATGTGGAAGATAGAAA | 3UTR319-L- |
| 107 | AAGCCTTTGGGGTCAGGCAGACTTGGATTCTAATACCAGCCAGTTC | 3UTR325 |
| | TGCTTGCTGTGTCTGAGCCTCAGTTTACTCATCTGTGAAGAGGAGG | |
| | TAGCAAGAATGAAAATGCCTGCCTTGTGGTTTGTTGTAAGGACAG | |
| | ACACTGCCAACGTAGAGGGCCCAGCAGCTCACAGACCAGTTGCTC | |
| | TGAGAGCAGAAAACAACAACAACAACAACTCCAACTCACGCTCTT | |
| | TGGAGCACAGAGAACTTTGATTTTTAAGCTTCTACAGATATGGTCC | |
| | TAGAGACTGGGGTCCTCAGACACTCCCGCTGTATGTGGTGAGCTTG | |
| | GCTGGAAACTTGCCATAGCCAGGACTGAGGGGCTGGCCCCAGGAC | |
| | GCTCTCAAGAAATGGGGCTACTGTGGTTTAGACACTCCTGCTGCTC | |
| | ACATTGATGGGTGGCTATTAAAG | |
| SEQ ID | TACTTAATAAATATTTGCTGTTGATGATAGCAATGACCTTGAGACT | 3UTR501; |
| NO: | GATGAACAGTCTGGCCAAGAGGATCCTTGATGTGGAAGATAGAAA | 3UTR319-L- |
| 108 | AAGCCTTTGGGGTCAGGCAGACTTGGATTCTAATACCAGCCAGTTC | 3UTR488 |
| | TGCTTGCTGTGTCTGAGCCTCAGTTTACTCATCTGTGAAGAGGAGG | |
| | TAGCAAGAATGAAAATGCCTGCCTTGTGGTTTGTTGTAAGGACAG | |
| | ACACTGCCAACGTAGAGGGCCCAGCAGCTCACAGACCAGTTGCTC | |
| | TGAGAGCAGAAAACAACAACAACAACAACTCCAACTCACCTTTGA | |
| | TTTTTAAGCTTCTACAGATATGGTCCTAGAGACTGGGGTCCTCAGA | |
| | CACTCCCGCTGTATGTGGTGAGCTTGGCTGGAAACTTGCCATAGCC | |
| | AGGACTGAGGGGCTGGCCCCAGGACGCTCTCAAGAAATGGGGCTA | |
| | CTGTGGTTTAGACACTCC | |
| 5′- | AGGGTTTCGTCTTTAGATCTTCTGGTCCCCACAGACTCAGAGAGAA | 5UTR025 |
| UTRs | CCCGCCACC | |
| SEQ ID | AGGGAGGCGACCGGAAGGATCTTTCTAGTCCAGCCCCTCGCTTTAC | 5UTR135 |
| NO: | CCGGACGAAAGACACGGGCCTGATTCGTCGAGTCTCACTGAGCCT | |
| 109 | TAGTCGTCGGCAGGTCCCAGGCGCGAAGTTTCTCGGCCTGGAGGA | |
| | GGGGGTCGCGCGAAGTGCCAGCCGCCACC | |
| SEQ ID | AGGGTCTGAGCGCCCGGCGGAAAACCGAAGTTGGAAGTGTCTCTT | 5UTR134 |
| NO: | AGCAGCGCGCGGAGAAGAACGGGGAGCCAGCATCCCGCCACC | |
| 110 | | |
| SEQ ID | AGGGACTTCGAGAGGGACTTAGAGAAGGCAGACGCATCCCGAACT | 5UTR136 |
| NO: | CGCTGGAGGACAAGGCTCAGCTCTTGCCAGGCCAAATTGAGACCC | |
| 111 | GCCACC | |
| SEQ ID | AGGGTGCCTCTGTTTGGCGCTTTTGTGCGCGCCCGGGTCTGTTGGT | 5UTR137 |
| NO: | GCTCAGAGTGTGGTCAGGCGGCTCGGACTGAGCAGGACTTTCCTTA | |
| 112 | TCCCAGTTGATTGTGCAGAATACACTGCCTGTCGCTTGTCTTCTATT | |
| | CACCCCGCCACC | |
| 3′- | | |
| UTRs | | |
| Trunc- | | |
| ations | | |
| SEQ ID | GGACACATGGTTACCACCTGGCCCTGAGTGCAGTCAGACTGGGAC | 3UTR155 |
| NO: | AAAGGAGCCGTGAAACACGCAAGGAGCTTCTGGCTTCTCAGCTTT | |
| 117 | GAGACCTGGGCTCTTTGGAGCACAGAGAACTTTGATTTTTAAGCTT | |
| | CTACAGATATGGTCCTAGAGACTGGGGTCCTCAGACACTCCCGCTG | |
| | TATGTGGTGAGCTTGGCTGGAAACTTGCCATAGCCAGGACTGAGG | |
| | GGCTGGCCCCAGGACGCTCTCAAGAAATGGGGCTACTGTGGTTTA | |
| | GACACTCCTGCTGCTCACATTGATGGGTGGCTATTAAAG | |
| SEQ ID | GCTCTTTGGAGCACAGAGAACTTTGATTTTTAAGCTTCTACAGATA | 3UTR325 |
| NO: | TGGTCCTAGAGACTGGGGTCCTCAGACACTCCCGCTGTATGTGGTG | |
| 114 | AGCTTGGCTGGAAACTTGCCATAGCCAGGACTGAGGGGCTGGCCC | |
| | CAGGACGCTCTCAAGAAATGGGGCTACTGTGGTTTAGACACTCCTG | |
| | CTGCTCACATTGATGGGTGGCTATTAAAG | |
| SEQ ID | GCTCTTTGGAGCACAGAGAACTTTGATTTTTAAGCTTCTACAGATA | 3UTR343 |
| NO: | TGGTCCTAGAGACTGGGGTCCTCAGACACTCCCGCTGTATGTGGTG | |
| 115 | AGCTTGGCTGGAAACTTGCCATAGCCAGGACTGAGGGGCTGGCCC | |
| | CAGGACGCTCTCAAGAAATGGGGCTACTGTGGTTTAGACACTCC | |
| SEQ ID | CTTTGATTTTTAAGCTTCTACAGATATGGTCCTAGAGACTGGGGTC | 3UTR488 |
| NO: | CTCAGACACTCCCGCTGTATGTGGTGAGCTTGGCTGGAAACTTGCC | |
| 116 | ATAGCCAGGACTGAGGGGCTGGCCCCAGGACGCTCTCAAGAAATG | |
| | GGGCTACTGTGGTTTAGACACTCC | |
| 3′ | | |
| UTR | | |
| Combined | | |
| with | | |
| other | | |
| 3′ | | |
| UTR | | |
| deriva- | | |
| tive | | |
| SEQ ID | TCCCAAATCCTGCCCCGTTGGCACAGGGCCA | 3UTR316 |
| NO: | | |
| 117 | | |
| SEQ ID | CACCACGTGCCAGAGCCGCCCACCCGGAGCCGCCCGCATGCAGCT | 3UTR317 |
| NO: | TCACCTCCCCTTTCCAGGCGCCACTGTTGAGAAGCTAGAGATTGTA | |
| 118 | TGAGAATAAACTTGTTATTATGGAAGCCTGGCTCCCACCCCATCTA | |
| SEQ ID | ATTTTAGGTATCTGCCTTGGTTTCAGTGGGGACATCTGGGGCTTAT | 3UTR318 |
| NO: | GGGGCTGGGATGAGGAGCTGGATGATTCTAGGAAGGCCCAAGTTG | |
| 119 | GAGAATGATGTGGAGAGTGTGCCAAGACACTGCTTTTGGCATTTTA | |
| | TTCCTTTCTGTTTGCTGGATGTCAATTGACCCTTTTATTTTCTCTTAC | |
| | TTGTGTTTTCAGATATTGTTAATCCTGCCAGTCTTTCTCTTCAAGCC | |
| | AGGGTGCATCCTCAGAAACCTACTCAACACAGCACTCTAGGCAGC | |
| | CACTATCAATCAATTGAAGTTGACACTCTGCATTAAATCTATTTGC | |
| | C | |
| SEQ ID | TACTTAATAAATATTTGCTGTTGATGATAGCAATGACCTTGAGACT | 3UTR319 |
| NO: | GATGAACAGTCTGGCCAAGAGGATCCTTGATGTGGAAGATAGAAA | |
| 120 | AAGCCTTTGGGGTCAGGCAGACTTGGATTCTAATACCAGCCAGTTC | |
| | TGCTTGCTGTGTCTGAGCCTCAGTTTACTCATCTGTGAAGAGGAGG | |
| | TAGCAAGAATGAAAATGCCTGCCTTGTGGTTTGTTGTAAGGACAG | |
| | ACACTGCCAACGTAGAGGGCCCAGCAGCTCACAGACCAGTTGCTC | |
| | TGAGAGCAG | |
| SEQ ID | TACTTAATAAATATTTGCTGTTGATGATAGCAATGACCTTGAGACT | 3UTR319 |
| NO: | GATGAACAGTCTGGCCAAGAGGATCCTTGATGTGGAAGATAGAAA | |
| 121 | AAGCCTTTGGGGTCAGGCAGACTTGGATTCTAATACCAGCCAGTTC | |
| | TGCTTGCTGTGTCTGAGCCTCAGTTTACTCATCTGTGAAGAGGAGG | |
| | TAGCAAGAATGAAAATGCCTGCCTTGTGGTTTGTTGTAAGGACAG | |
| | ACACTGCCAACGTAGAGGGCCCAGCAGCTCACAGACCAGTTGCTC | |
| | TGAGAGCAG | |
| SEQ ID | ATTTTAGGTATCTGCCTTGGTTTCAGTGGGGACATCTGGGGCTTAT | 3UTR318 |
| NO: | GGGGCTGGGATGAGGAGCTGGATGATTCTAGGAAGGCCCAAGTTG | |
| 122 | GAGAATGATGTGGAGAGTGTGCCAAGACACTGCTTTTGGCATTTTA | |
| | TTCCTTTCTGTTTGCTGGATGTCAATTGACCCTTTTATTTTCTCTTAC | |
| | TTGTGTTTTCAGATATTGTTAATCCTGCCAGTCTTTCTCTTCAAGCC | |
| | AGGGTGCATCCTCAGAAACCTACTCAACACAGCACTCTAGGCAGC | |
| | CACTATCAATCAATTGAAGTTGACACTCTGCATTAAATCTATTTGC | |
| | C | |
| SEQ ID | GGACACATGGTTACCACCTGGCCCTGAGTGCAGTCAGACTGGGAC | 3UTR155 |
| NO: | AAAGGAGCCGTGAAACACGCAAGGAGCTTCTGGCTTCTCAGCTTT | |
| 123 | GAGACCTGGGCTCTTTGGAGCACAGAGAACTTTGATTTTTAAGCTT | |
| | CTACAGATATGGTCCTAGAGACTGGGGTCCTCAGACACTCCCGCTG | |
| | TATGTGGTGAGCTTGGCTGGAAACTTGCCATAGCCAGGACTGAGG | |
| | GGCTGGCCCCAGGACGCTCTCAAGAAATGGGGCTACTGTGGTTTA | |
| | GACACTCCTGCTGCTCACATTGATGGGTGGCTATTAAAG | |
| SEQ ID | GGACACATGGTTACCACCTGGCCCTGAGTGCAGTCACGCGTACCA | 3UTR578 |
| NO: | AAAGTAATAATGGACTGGGACAAAGGAGCCGTGAAACACGCAAG | |
| 124 | GAGCTTCTGGCTTCTCAGCTTTGAGACCTGGGCTCTTTGGAGCACA | |
| | GAGAACTTTGATTTTTAAGCTTCTACAGATATGGTCCTAGAGACTG | |
| | GGGTCCTCAGACACTCCCGCTGTATGTGGTGAGCTTGGCTGGAAAC | |
| | TTGCCATAGCCAGGACTGAGGGGCTGGCCCCAGGACGCTCTCAAG | |
| | AAATGGGGCTACTGTGGTTTAGACACTCCTGCTGCTCACATTGATG | |
| | GGTGGCTATTAAAG | |
| SEQ ID | GGACACATGGTTACCACCTGGCCCTGAGTGCAGTCAGACTGGGAC | 3UTR579 |
| NO: | AAAGGAGCCGTGAAACACGCAAGGAGCTTCTGGCTTCTCAGCTTT | |
| 125 | GAGACCTGGGCTCTTTGGAGCACAGAGAACTTTGATTTTTAAGCTT | |
| | CTACAGATATGGTCCTAGAGACTGGGGTCCTCAGACACTCCCGCTG | |
| | TATGTGGTGAGCTTGGCTGGAAACTTGCCATCGCGTACCAAAAGTA | |
| | ATAATGAGCCAGGACTGAGGGGCTGGCCCCAGGACGCTCTCAAGA | |
| | AATGGGGCTACTGTGGTTTAGACACTCCTGCTGCTCACATTGATGG | |
| | GTGGCTATTAAAG | |
| SEQ ID | ATTTTAGGTATCTGCCTTGGTTTCAGTGGGGACATCTGGGGCTTAT | 3UTR580 |
| NO: | GGGGCTGGGATGAGGAGCTGGATGATTCTAGGAAGGCCCAAGTTG | |
| 126 | GAGAATGATGTGGAGAGTGTGCCAAGACGCGTACCAAAAGTAATA | |
| | ATGCACTGCTTTTGGCATTTTATTCCTTTCTGTTTGCTGGATGTCAA | |
| | TTGACCCTTTTATTTTCTCTTACTTGTGTTTTCAGATATTGTTAATCC | |
| | TGCCAGTCTTTCTCTTCAAGCCAGGGTGCATCCTCAGAAACCTACT | |
| | CAACACAGCACTCTAGGCAGCCACTATCAATCAATTGAAGTTGAC | |
| | ACTCTGCATTAAATCTATTTGCC | |
| SEQ ID | ATTTTAGGTATCTGCCTTGGTTTCAGTGGGGACATCTGGGGCTTAT | 3UTR581 |
| NO: | GGGGCTGGGATGAGGAGCTGGATGATTCTAGGAAGGCCCAAGTTG | |
| 127 | GAGAATGATGTGGAGAGTGTGCCAAGACACTGCTTTTGGCATTTTA | |
| | TTCCTTTCTGTTTGCTGGATGTCAATTGACCCTTTTATTTTCTCTTAC | |
| | TTGTGTTTTCAGATATTGTTAATCCTGCCAGTCTTTCTCTTCAAGCC | |
| | AGGGTGCATCCTCAGAACGCGTACCAAAAGTAATAATGACCTACT | |
| | CAACACAGCACTCTAGGCAGCCACTATCAATCAATTGAAGTTGAC | |
| | ACTCTGCATTAAATCTATTTGCC | |
| SEQ ID | ATTTTAGGTATCTGCCTTGGTTTCAGTGGGGACATCTGGGGCTTAT | 3UTR582 |
| NO: | GGGGCTGGGATGAGGAGCTGGATGATTCTAGGAAGGCCCAAGTTG | |
| 128 | GAGAATGATGTGGAGAGTGTGCCAAGACACTGCTTTTGGCATTTTA | |
| | TTCCTTTCTGTTTGCTGGATGTCAATTGACCCTTTTATTTTCTCTTAC | |
| | TTGTGTTTTCAGATATTGTTAATCCTGCCAGTCTTTCTCTTCAAGCC | |
| | AGGGTGCATCCTCAGAAACCTACTCAACACAGCACTCTAGGCAGC | |
| | CACTATCAATCAATTGAAGTTGACACTCTGCATTAAATCTCGCGTA | |
| | CCAAAAGTAATAATGATTTGCC | |
| SEQ ID | TCGCGTACCAAAAGTAATAATGACTTAATAAATATTTGCTGTTGAT | 3UTR583 |
| NO: | GATAGCAATGACCTTGAGACTGATGAACAGTCTGGCCAAGAGGAT | |
| 129 | CCTTGATGTGGAAGATAGAAAAAGCCTTTGGGGTCAGGCAGACTT | |
| | GGATTCTAATACCAGCCAGTTCTGCTTGCTGTGTCTGAGCCTCAGT | |
| | TTACTCATCTGTGAAGAGGAGGTAGCAAGAATGAAAATGCCTGCC | |
| | TTGTGGTTTGTTGTAAGGACAGACACTGCCAACGTAGAGGGCCCA | |
| | GCAGCTCACAGACCAGTTGCTCTGAGAGCAG | |
| SEQ ID | TACTTAATAAATATTTGCTGTTGATGATAGCAATGACCTTGAGACT | 3UTR584 |
| NO: | GCGCGTACCAAAAGTAATAATGATGAACAGTCTGGCCAAGAGGAT | |
| 130 | CCTTGATGTGGAAGATAGAAAAAGCCTTTGGGGTCAGGCAGACTT | |
| | GGATTCTAATACCAGCCAGTTCTGCTTGCTGTGTCTGAGCCTCAGT | |
| | TTACTCATCTGTGAAGAGGAGGTAGCAAGAATGAAAATGCCTGCC | |
| | TTGTGGTTTGTTGTAAGGACAGACACTGCCAACGTAGAGGGCCCA | |
| | GCAGCTCACAGACCAGTTGCTCTGAGAGCAG | |
| SEQ ID | TACTTCGCGTACCAAAAGTAATAATGAATAAATATTTGCTGTTGAT | 3UTR585 |
| NO: | GATAGCAATGACCTTGAGACTGATGAACAGTCTGGCCAAGAGGAT | |
| 131 | CCTTGATGTGGAAGATAGAAAAAGCCTTTGGGGTCAGGCAGACTT | |
| | GGATTCTAATACCAGCCAGTTCTGCTTGCTGTGTCTGAGCCTCAGT | |
| | TTACTCATCTGTGAAGAGGAGGTAGCAAGAATGAAAATGCCTGCC | |
| | TTGTGGTTTGTTGTAAGGACAGACACTGCCAACGTAGAGGGCCCA | |
| | GCAGCTCACAGACCAGTTGCTCTGAGAGCAG | |
| SEQ ID | TACTTAATAAATATTTGCTGTTGATGATAGCAATGACCTTGAGACT | 3UTR592 |
| NO: | GATGAACAGTCTGGCCAAGAGGATCCTTGATGTGGAAGATAGAAA | |
| 132 | AAGCCTTTGGGGTCAGGCAGACTTGGATTCTAATACCAGCCAGTTC | |
| | TGCTTGCTGTGTCTGAGCCTCAGTTTACTCATCTGTGAAGAGGAGG | |
| | TAGCAAGAATGAAAATGCCTGCCTTGTGGTTTGTTGTAAGGACAG | |
| | ACACTGCCAACGTAGAGGGCCCAGCAGCTCACAGACCAGTTGCTC | |
| | TGAGAGCAGAAAACAACAACAACAACAACTCCAACTCACGCTCTT | |
| | TGGAGCACAGAGAACTTTGATTTTTAAGCTTCTACAGATATGGTCC | |
| | TAGAGACTGGGGTCCTCAGACACTCCCGCTGTATGTGGTGAGCTTG | |
| | GCTGGAAACTTGCCATCGCGTACCAAAAGTAATAATGAGCCAGGA | |
| | CTGAGGGGCTGGCCCCAGGACGCTCTCAAGAAATGGGGCTACTGT | |
| | GGTTTAGACACTCCTGCTGCTCACATTGATGGGTGGCTATTAAAG | |
| SEQ ID | GCTCTTTGGAGCACAGAGAACTTTGATTTTTAAGCTTCTACAGATA | 3UTR593 |
| NO: | TGGTCCTAGAGACTGGGGTCCTCAGACACTCCCGCTGTATGTGGTG | |
| 133 | AGCTTGGCTGGAAACTTGCCATAGCCAGGACTGAGGGGCTGGCCC | |
| | CAGGACGCTCTCAAGAAATGGGGCTACTGTGGTTTAGACACTCCTG | |
| | CTGCTCACATTGATGGGTGGCTATTAAAGAAAACAACAACAACAA | |
| | CAACTCCAACTCACTACTTAATAAATATTTGCTGTTGATGATAGCA | |
| | ATGACCTTGAGACTGCGCGTACCAAAAGTAATAATGATGAACAGT | |
| | CTGGCCAAGAGGATCCTTGATGTGGAAGATAGAAAAAGCCTTTGG | |
| | GGTCAGGCAGACTTGGATTCTAATACCAGCCAGTTCTGCTTGCTGT | |
| | GTCTGAGCCTCAGTTTACTCATCTGTGAAGAGGAGGTAGCAAGAA | |
| | TGAAAATGCCTGCCTTGTGGTTTGTTGTAAGGACAGACACTGCCAA | |
| | CGTAGAGGGCCCAGCAGCTCACAGACCAGTTGCTCTGAGAGCAG | |
| SEQ ID | GGACACATGGTTACCACCTGGCCCTGAGTGCAGTCAGACTGGGAC | 3UTR598 |
| NO: | AAAGGAGCCGTGAAACACGCAAGGAGCTTCTGGCTTCTCAGCTTT | |
| 134 | GAGACCTGGGCTCTTTGGAGCACAGAGAACTTTGATTTTTAAGCTT | |
| | CTACAGATATGGTCCTAGAGACTGGGGTCCTCAGACACTCCCGCTG | |
| | TATGTGGTGAGCTTGGCTGGAAACTTGCCATCGCGTACCAAAAGTA | |
| | ATAATGAGCCAGGACTGAGGGGCTGGCCCCAGGACGCTCTCAAGA | |
| | AATGGGGCTACTGTGGTTTAGACACTCCTGCTGCTCACATTGATGG | |
| | GTGGCTATTAAAGAAAACAACAACAACAACAACTCCAACTCACTA | |
| | CTTAATAAATATTTGCTGTTGATGATAGCAATGACCTTGAGACTGA | |
| | TGAACAGTCTGGCCAAGAGGATCCTTGATGTGGAAGATAGAAAAA | |
| | GCCTTTGGGGTCAGGCAGACTTGGATTCTAATACCAGCCAGTTCTG | |
| | CTTGCTGTGTCTGAGCCTCAGTTTACTCATCTGTGAAGAGGAGGTA | |
| | GCAAGAATGAAAATGCCTGCCTTGTGGTTTGTTGTAAGGACAGAC | |
| | ACTGCCAACGTAGAGGGCCCAGCAGCTCACAGACCAGTTGCTCTG | |
| | AGAGCAG | |
| SEQ ID | TACTTAATAAATATTTGCTGTTGATGATAGCAATGACCTTGAGACT | 3UTR594 |
| NO: | GCGCGTACCAAAAGTAATAATGATGAACAGTCTGGCCAAGAGGAT | |
| 135 | CCTTGATGTGGAAGATAGAAAAAGCCTTTGGGGTCAGGCAGACTT | |
| | GGATTCTAATACCAGCCAGTTCTGCTTGCTGTGTCTGAGCCTCAGT | |
| | TTACTCATCTGTGAAGAGGAGGTAGCAAGAATGAAAATGCCTGCC | |
| | TTGTGGTTTGTTGTAAGGACAGACACTGCCAACGTAGAGGGCCCA | |
| | GCAGCTCACAGACCAGTTGCTCTGAGAGCAGAAAACAACAACAAC | |
| | AACAACTCCAACTCACGCTCTTTGGAGCACAGAGAACTTTGATTTT | |
| | TAAGCTTCTACAGATATGGTCCTAGAGACTGGGGTCCTCAGACACT | |
| | CCCGCTGTATGTGGTGAGCTTGGCTGGAAACTTGCCATAGCCAGGA | |
| | CTGAGGGGCTGGCCCCAGGACGCTCTCAAGAAATGGGGCTACTGT | |
| | GGTTTAGACACTCCTGCTGCTCACATTGATGGGTGGCTATTAAAG | |
| SEQ ID | GCTCTTTGGAGCACAGAGAACTTTGATTTTTAAGCTTCTACAGATA | 3UTR596 |
| NO: | TGGTCCTAGAGACTGGGGTCCTCAGACACTCCCGCTGTATGTGGTG | |
| 136 | AGCTTGGCTGGAAACTTGCCATCGCGTACCAAAAGTAATAATGAG | |
| | CCAGGACTGAGGGGCTGGCCCCAGGACGCTCTCAAGAAATGGGGC | |
| | TACTGTGGTTTAGACACTCCTGCTGCTCACATTGATGGGTGGCTAT | |
| | TAAAGAAAACAACAACAACAACAACTCCAACTCACTACTTAATAA | |
| | ATATTTGCTGTTGATGATAGCAATGACCTTGAGACTGATGAACAGT | |
| | CTGGCCAAGAGGATCCTTGATGTGGAAGATAGAAAAAGCCTTTGG | |
| | GGTCAGGCAGACTTGGATTCTAATACCAGCCAGTTCTGCTTGCTGT | |
| | GTCTGAGCCTCAGTTTACTCATCTGTGAAGAGGAGGTAGCAAGAA | |
| | TGAAAATGCCTGCCTTGTGGTTTGTTGTAAGGACAGACACTGCCAA | |
| | CGTAGAGGGCCCAGCAGCTCACAGACCAGTTGCTCTGAGAGCAG | |
| SEQ ID | GCTCTTTGGAGCACAGAGAACTTTGATTTTTAAGCTTCTACAGATA | 3UTR597 |
| NO: | TGGTCCTAGAGACTGGGGTCCTCAGACACTCCCGCTGTATGTGGTG | |
| 137 | AGCTTGGCTGGAAACTTGCCATCGCGTACCAAAAGTAATAATGAG | |
| | CCAGGACTGAGGGGCTGGCCCCAGGACGCTCTCAAGAAATGGGGC | |
| | TACTGTGGTTTAGACACTCCTGCTGCTCACATTGATGGGTGGCTAT | |
| | TAAAGAAAACAACAACAACAACAACTCCAACTCACTACTTAATAA | |
| | ATATTTGCTGTTGATGATAGCAATGACCTTGAGACTGCGCGTACCA | |
| | AAAGTAATAATGATGAACAGTCTGGCCAAGAGGATCCTTGATGTG | |
| | GAAGATAGAAAAAGCCTTTGGGGTCAGGCAGACTTGGATTCTAAT | |
| | ACCAGCCAGTTCTGCTTGCTGTGTCTGAGCCTCAGTTTACTCATCTG | |
| | TGAAGAGGAGGTAGCAAGAATGAAAATGCCTGCCTTGTGGTTTGT | |
| | TGTAAGGACAGACACTGCCAACGTAGAGGGCCCAGCAGCTCACAG | |
| | ACCAGTTGCTCTGAGAGCAG | |
| SEQ ID | AGGGTTTCGTCTTTAGATCTTCTGGTCCCCACAGACTCAGAGAGAA | 5UTR025 |
| NO: | CCCGCCACC | |
| 138 | | |
| SEQ ID | AGGGACTTCGAGAGGGACTTAGAGAAGGCAGACGCATCCCGAACT | 5UTR136 |
| NO: | CGCTGGAGGACAAGGCTCAGCTCTTGCCAGGCCAAATTGAGACCC | |
| 139 | GCCACC | |
| SEQ ID | GGCGTTGACCGCGAAGGACGAGGCGTCCCCGTGACGTCCTGCTCTT | 5UTR176 |
| NO: | CCAATGAACTTCAGGCCTGCGGAGACGACGGAGAACGGAAG | |
| 140 | | |
| SEQ ID | GGCGTTGACCGCGAAGGACGAGGCGTCCCCGTGACGTCCTGCTCTTCCAATGAAC | 5UTR177 |
| NO: | TTCAGGCCTGCGGAGACGACGGAGAACGGAAGTTCTCTGCCTGTGTGCTGGTTGG | |
| 141 | TTGCGCGTTGAGGCCAGCCCCGCCTCCCATTTTCCGGTCTCCTCAGAAGTCGCTTAG | |
| | CTCTTCGGTGGTTGTCCCACGTCCGGAGGCCTAGCCGTCGCTTACCTAGG | |
| SEQ ID | CCTATAACTTGGAATGTGGGTGGAGGGGTTCATAGTTCTCCCTGAGTGAGACTTGC | 5UTR178 |
| NO: | CTGCTTCTCTGGCCCCTGGTCCTGTCCTGTTCTCCAGC | |
| 142 | | |
| SEQ ID | CCTATAACTTGGAATATGGGTGGAGGGGTTCATAGTTCTCCCTCAGTGAGACTTGC | 5UTR179 |
| NO: | CTGCTTCTCTGGCCCCTGGTCCTGTCGTGTTCTCCAGC | |
| 143 | | |
| SEQ ID | GGGAGACTGAGGACTTGGATTGCTTAAGGACACCAGGCAGGCGCGACAGCCTTCA | 5UTR180 |
| NO: | GCCTCTCCCTCTGCAGTTTTGGCCGGTTCCTTTAACTTGCCTTCATCTGGGGCTGTTT | |
| 144 | GGTATTTCCACGTGCCTTGGGCCCGCCCACTGCAGCCTCCATCTTGGAAGCGGCCG | |
| | CCGGCGCCTAGATTGAGCATTTCCACAGAAATTACAGTTTTGTCCTTTTTGAAAAAA | |
| | TAGAACTGTATTTCAGAAAAAAGAAACTACAGTTTTAGCATGCAGAAAGGAAAAG | |
| | GGAGAACAAGCCGGATCAGAAGACGAAAACTCTGCGGAAGTTCTGAATCAAGAG | |
| | GAGAGATGGGGTCTTTCTGTGTTGGGCAGGCTGGGCTCAAACTCCTGGCCTCAAG | |
| | CAGTCCTCCCGCCTTGGCCTCCCAAATGCTGGGAATACAGA | |
| SEQ ID | GGGAGACTGAGGACTTGGATTGCTTAAGGACACCAGGCAGGCGCGACAGCCTTCA | 5UTR181 |
| NO: | GCCTCTCCCTCTGCAGTTTTGGCCGGTTCCTTTAACTTGCCTTCATCTGGGGCTGTTT | |
| 145 | GGTATTTCCACGTGCCTTGGGCCCGCCCACTGCAGCCTCCATCTTGGAAGCGGCCG | |
| | CCGGCGCCTAGATTGAGCATTTCCACAGAAATTACAGTTTTGTCCTTTTTGAAAAAA | |
| | TAGAACTGTATTTCAGAAAAAAGAAACTACAGTTTTAGCTTGCAGAAAGGAAAAG | |
| | GGAGAACAAGCCGGATCAGAAGACGAAAACTCTGCGGAAGTTCTGAATCAAGAG | |
| | GAGAGATGGGGTCTTTCTGTGTTGGGCAGGCTGGGCTCAAACTCCTGGCCTCAAG | |
| | CAGTCCTCCCGCCTTGGCCTCCCAAATGCTGGGAATACAGA | |
| SEQ ID | cctataacttggaatgtgggtggaggggttcatagttctccctgagtgagacttgcctgctt | 5UTR182 |
| NO: | ctctggcccctggtcctgtcgtgttctccagc | |
| 146 | | |

1. An engineered polynucleotide comprising:
at least one oncoselective sequence; and
a coding sequence encoding an engineered therapeutic, wherein the at least one oncoselective sequence comprises a nucleic acid sequence that is:
at least 75% identical to any one of SEQ ID NOs: 1-97 or 101-146.

2. (canceled)

3. (canceled)

4. The engineered polynucleotide of claim 1, wherein the at least one oncoselective sequence comprises the nucleic acid sequence that is at least 50 contiguous nucleotides, at least 100 contiguous nucleotides, or at least 150 contiguous nucleotides identical to any one of SEQ ID NOs: 1-75, 91-97, or 101-146.

5. The engineered polynucleotide of claim 1, wherein the at least one oncoselective sequence comprises the nucleic acid sequence that is at least 5 contiguous nucleotides identical to any one of SEQ ID NOs: 76-90.

6. The engineered polynucleotide of claim 1, wherein the at least one oncoselective sequence comprises an untranslated region (UTR).

7. The engineered polynucleotide of claim 1, wherein the at least one oncoselective sequence comprises a nucleic acid motif.

8. The engineered polynucleotide of claim 1, further comprising two oncoselective sequences.

9. The engineered polynucleotide of claim 8, wherein the two oncoselective sequences flank the coding sequence.

10. The engineered polynucleotide of claim 9, wherein the two oncoselective sequences each comprises the nucleic acid sequence that is: at least 75% identical to any one of SEQ ID NOs: 1-75, 91-97, or 101-14; or any one of SEQ ID NOs: 76-90.

11. The engineered polynucleotide of claim 1, wherein the at least one oncoselective sequence comprises at least one miRNA binding site or at least one protein binding site.

12. (canceled)

13. The engineered polynucleotide of claim 1, further comprising at least one nucleic acid modification.

14. (canceled)

15. (canceled)

16. (canceled)

17. The engineered polynucleotide of claim 1, wherein the engineered therapeutic comprises a cytokine.

18. The engineered polynucleotide of claim 17, wherein the engineered therapeutic is encoded from a nucleic acid sequence that is at least 75%, at least 80%, at least 85%, at least 90%, at least 95%, or at least 99% identical to any one of SEQ ID NOs: 461476.

19. The engineered polynucleotide of claim 17, wherein the engineered therapeutic comprises an amino acid sequence that is at least 75%, at least 80%, at least 85%, at least 90%, at least 95%, or at least 99% identical to any one of SEQ ID NOs: 301-303, 311-314, 321-328, 331-335, 341-344, 351-361, 371-373, 481-490, or 501-510.

20. The engineered polynucleotide of claim 17, wherein the cytokine comprises an engineered cytokine comprising a secreted cytokine, a membrane tethered cytokine, a masked cytokine, a cytokine fusion, or a combination thereof.

21. The engineered polynucleotide of claim 17, wherein the cytokine comprises an interleukin or an interferon.

22.-50. (canceled)

51. The engineered polynucleotide of claim 1, wherein the at least one oncoselective sequence increases an expression of the engineered therapeutic in a cancer cell compared to a comparable expression of the engineered therapeutic in a non-cancer cell.

52. (canceled)

53. The engineered polynucleotide of claim 1, wherein the at least one oncoselective sequence increases a killing efficiency of a cancer cell compared to a comparable killing efficiency of a non-cancer cell.

54. A composition comprising the engineered polynucleotide of claim 1.

55.-61. (canceled)

62. The composition of claim 54, wherein the composition comprises contacting the engineered polynucleotide with a lipid.

63.-73. (canceled)

74. A method for treating a disease or condition in a subject, comprising administering to the subject an engineered polynucleotide, said engineered polynucleotide comprises at least one oncoselective sequence; and a coding sequence encoding an engineered therapeutic, wherein the at least one oncoselective sequence comprises a nucleic acid sequence that is:
at least 75% identical to any one of SEQ ID NOs: 1-97 or 101-146.

75.-101. (canceled)
